(19) United States
(12) Patent Application Publication
(10) Pub. No.: US 2003/0154032 A1
(43) Pub. Date: Aug. 14, 2003

(54) Methods and compositions for diagnosing and treating rheumatoid arthritis

(76) Inventors: Debra D. Pittman, Windham, NH (US); Jeffrey L. Feldman, Arlington, MA (US); Kathleen M. Shields, Harvard, MA (US); William L. Trepicchio, Andover, MA (US)

Correspondence Address: Patent Group, FOLEY, HOAG & ELIOT LLP, One Post Office Square, Boxton, MA, 02109, US

(21) Appl. No.: 10/023,451

(22) Filed: Dec. 17, 2001

Related U.S. Application Data
(60) Provisional application No. 60/255,861, filed on Dec. 15, 2000.

(51) Int. Cl. G06F 19/00; G01N 33/48

(52) U.S. Cl. 702/20

(57) ABSTRACT

The invention provides methods and compositions for diagnostic assays for detecting R.A. and therapeutic methods and compositions for treating R.A. The invention also provides methods for designing, identifying, and optimizing therapeutics for R.A. Diagnostic compositions of the invention include compositions comprising detection agents for detecting one or more genes that have been shown to be up- or down-regulated in cells of R.A. relative to normal counterpart cells. Exemplary detection agents include nucleic acid probes, which can be in solution or attached to a solid surface, e.g., in the form of a microarray. The invention also provides computer-readable media comprising values of levels of expression of one or more genes that are up- or down-regulated in R.A.

### CROSS-REFERENCE TO RELATED APPLICATIONS

This application claims the benefit of U.S. Provisional Application No. 60/255,861, filed Dec. 15, 2000, the contents of which are specifically incorporated by reference herein.

### BACKGROUND OF THE INVENTION

Inflammatory reactions are the cause of a significant number of diseases or disorders, some of which lack appropriate methods of treatment. For example, rheumatoid arthritis (R.A.) is a systematic inflammatory disease that commonly affects the joints, particularly those of the hands and feet. The onset of rheumatoid arthritis can occur slowly, ranging from a few weeks to a few months, or the condition can surface rapidly in an acute manner.

Today, over 2,500,000 individuals are diagnosed with rheumatoid arthritis in the United States alone (1% of population), with some statistics indicating from 6.5 to 8 million potentially afflicted with the disease. Women are affected 2-3 times more often than men. The disease can occur at any age and typically will increase in incidence with age. The classic early symptoms of rheumatoid arthritis include stiffness, tenderness, fever, subcutaneous nodules, achy joints, and fatigue. The joints of the hands, feet, knees and wrists are most commonly affected, with eventual involvement of the hips, elbows and shoulders. As the joints stiffen and swell, any type of motion becomes very painful and difficult. The more severe cases of rheumatoid arthritis can lead to intense pain and eventual joint destruction. Some 300,000 bone and joint replacement surgical procedures are performed annually in an effort to alleviate the pain and mobility loss resultant from arthritis related joint destruction.

The effective treatment of rheumatoid arthritis has generally comprised a combination of medication, exercise, rest and proper joint protection therapy. The therapy for a particular patient depends on the severity of the disease and the joints that are involved. Aspirin is widely used for pain and to reduce inflammation. In addition to aspirin, non-steroidal anti-inflammatory drugs, corti-costeroids, gold salts, anti-malarials and systemic immunosuppressants are widely used in moderate to advanced cases. The use of steroids and immunosuppressants, however, has significant risks and side effects both in terms of toxicity and vulnerability to potentially lethal conditions.

There, thus exists a need for methods of diagnosing and treating inflammatory diseases, e.g., rheumatoid arthritis, which do not entail the potentially lethal side effects associated with the treatments described above.

### SUMMARY OF THE INVENTION

In one embodiment, the invention provides diagnostic methods, composition and devices for monitoring and/or predicting the existence, development and/or evolution of R.A. in a subject. Preferred methods comprise determining levels of expression of one or more genes characteristic of R.A. in a cell and comparing these to the levels of expression of these genes in other cells.

Comparison of the expression levels can be performed visually. In a preferred embodiment, the comparison is performed by a computer. In one embodiment, expression levels of genes characteristic of R.A. in cells of subjects having R.A. are stored in a computer. The computer may optionally comprise expression levels of these genes in normal cells. The data representing expression levels of the genes in a patient being diagnosed are then entered into the -computer, and compared with one or more of the expression levels stored in the computer. The computer calculates differences and presents data showing the differences in expression of the genes in the two types of cells.

Accordingly, in one embodiment, the invention provides computer-readable media comprising a plurality of digitally encoded values representing the levels of expression of a plurality of genes which are up- or down-regulated in R.A. in a cell characteristic of R.A. In one embodiment, a computer-readable medium includes values representing levels of expression of one or more genes encoding kinases, phosphatases or genes which are located on chromosome 6, region p21.3, such as those highligheted in the Tables. In another embodiment, the computer-readable medium comprises values of levels of expression of a plurality of genes selected from the group consisting of SOCS3 (CISH3); RAGE (AGER); LST-1 (LY117); serum amyloid (SAA) 1-3; HMG-1; S100 A8, A9, and A12; Secretory Leukocyte Protease Inhibitor (SLPI); glucocorticoid leucine zipper (GILZ); PTPN-18; GADD-45A and B; Legumain (PRSC1); follistatin-like 1 (FST1); lipocalin 2 (Lcn2); glucose phosphate isomerase (GPI); Serine Protease Inhibitor (SpiL); and TSG-6. In a preferred embodiment, a computer-readable medium comprises values representing levels of expression of at least 5 of these genes. In another embodiment, a computer-readable medium comprises the levels of expression of at least 10 genes characteristic of R.A. in a cell characteristic of R.A. A computer-readable medium may also comprise values representing levels of expression of at least 50% of the genes set forth in Tables 1-5. Optionally, a computer-readable medium further comprises at least one value representing a level of expression of at least one gene characteristic of R.A. in a normal counterpart cell. The values on a computer-readable medium may represent ratios of, or differences between, a level of expression of a gene characteristic of R.A. in a cell characteristic of R.A. and a level of expression of the gene in a normal counterpart cell. In a preferred embodiment, less than about 50% of the values on the computer-readable medium represent expression levels of genes which are not characteristic of R.A.

The invention also provides computer systems, comprising a database comprising values representing expression levels of a plurality of genes which are up- or down-regulated in R.A., and including, e.g., one or more genes highlighted or marked with a star in the Tables, e.g., one or a plurality of genes selected from the group consisting of SOCS3 (CISH3); RAGE (AGER); LST-1 (LY117); SAA 1-3; HMG-1; S100 A8, A9, and A12; SLPI; GILZ; PTPN-18; GADD-45A and B; Legumain (PRSC1); FST1; Lcn2; GPI; SpiL; and TSG-6, in a cell characteristic of R.A.; and, a processor having instructions to, (i) receive at least one query value representing at least one level of expression of at least one gene represented in the database, and, (ii) compare the at least one query value and the at least one database value. The instructions to receive may include instructions to provide a user interface. The instructions may further include instructions to display at least one comparison and/or to create at least one record based on the comparison. The computer system may further including instructions to display the at least one record.

Also provided by the invention are computer programs for analyzing levels of expression of a plurality of genes characteristic of R.A. in a cell, the computer program being disposed on a computer readable medium and including instructions for causing a processor to (i) receive query values representing levels of expression of a plurality of genes characteristic of R.A. in a cell, and, (ii) compare the query values with levels of expression of the plurality of genes in a cell characteristic of R.A. The computer program may further comprise instructions to display at least one comparison. The instructions to compare may include instructions to retrieve the at least one level expression value from a computer readable medium and/or from a database. The instructions to receive may include instructions to provide a user interface.

In another embodiment, the invention provides computer programs for analyzing an expression profile of a cell characteristic of R.A. in a subject, the computer programs being disposed on a computer readable medium and including instructions for causing a processor to (i) receive at least one query expression profiles comprising a plurality of values, each value representing a level of expression of a gene characteristic of R.A. in a cell characteristic of R.A., and, (ii) compare the at least one query expression profile and at least one reference expression profile comprising a plurality of values, each value representing a level of expression of a gene characteristic of R.A. in a particular cell.

Also within the scope of the invention are compositions, such as compositions comprising a plurality of detection agents of genes which are up- or down-regulated in R.A., e.g., one or more genes highlighted or marked with a star in the Tables, e.g., one or a plurality of genes selected from the group consisting of SOCS3 (CISH3); RAGE (AGER); LST-1 (LY117); SAA 1-3; HMG-1; S1OO A8, A9, and A12; SLPI; GILZ; PTPN-18; GADD-45A and B; Legumain (PRSC1); FST1; Lcn2; GPI; SpiL; and TSG-6, which are capable of detecting the expression of the genes or the polypeptide encoded by the genes, and wherein less than about 50% of the detection agents are genes which are not characteristic of R.A. The detection agents may be isolated nucleic acids which hybridize specifically to nucleic acids corresponding to the genes. Compositions may comprise isolated nucleic acids which hybridize specifically to at least five genes selected from the group consisting of SOCS3 (CISH3); RAGE (AGER); LST-1 (LY117); SAA 1-3; HMG-1; S100 A8, A9, and A12; SLPI; GILZ; PTPN-18; GADD-45A and B; Legumain (PRSC1); FST1; Lcn2; GPI; SpiL; and TSG-6. In another embodiment, a composition may comprise isolated nucleic acids which hybridize specifically to at least 10 or 100 different genes characteristic of R.A. The detection agents may also detect the polypeptides encoded by the genes and may be, e.g., antibodies.

The invention also provides solid surfaces to which are linked a plurality of detection agents of genes which are up- or down-regulated in R.A., e.g., one or more genes highlighted or marked with a star in the Tables, e.g., one or a plurality of genes selected from the group consisting of SOCS3 (CISH3); RAGE (AGER); LST-1 (LY117); SAA 1-3; HMG-1; S100 A8, A9, and A12; SLPI; GILZ; PTPN-18; GADD-45A and B; Legumain (PRSC1); FST1; Lcn2; GPI; SpiL; and TSG-6, which detection agents are capable of detecting the expression of the genes or the polypeptide encoded by the genes, and wherein less than about 50% of the detection agents on the solid surface are not detecting genes characteristic of R.A. The detection agents may be isolated nucleic acids which hybridize specifically to the genes. The detection agents may be covalently linked to the solid surface.

Other compositions provided by the invention include compositions, such as pharmaceutical compositions comprising agonists or antagonists of a plurality of genes characteristic of R.A., such as antagonists of one or a plurality of genes selected from the group consisting of SOCS3 (CISH3); RAGE (AGER); LST-1 (LY117); SAA 1-3; HMG-1; S100 A8, A9, and A12; SLPI; GILZ; PTPN-18; GADD-45A and B; Legumain (PRSCl); FST1; Lcn2; GPI; SpiL; and TSG-6. Agonists may be polypeptides encoded by the genes or functional fragments or equivalents thereof, which may be fused to a transcytosis polypeptide. Agonists may also be genes encoding the polypeptides and the nucleic acids may be in one or more expression vectors. Antagonists may be antisense nucleic acids, siRNAs, ribozymes or dominant negative mutants.

The invention provides methods for determining the difference between levels of expression of a one or a plurality of genes characteristic of R.A. in a cell and reference levels of expression of the genes, comprising (i) providing RNA from a cell; (ii) determining levels of RNA of a plurality of genes genes which are up- or down-regulated in R.A., e.g., one or more genes highlighted or marked with a star in the Tables, e.g., one or a plurality of genes selected from the group consisting of SOCS3 (CISH3); RAGE (AGER); LST-1 (LY117); SAA 1-3; HMG-1; S100 A8, A9, and A12; SLPI; GILZ; PTPN-18; GADD-45A and B; Legumain (PRSC1); FST1; Lcn2; GPI; SpiL; and TSG-6 to obtain the levels of expression of the plurality of genes in the cell; and (iii) comparing the levels of expression of the plurality of genes in the cell to a set of reference levels of expression of the genes, to thereby determine the difference between levels of expression of the plurality of genes characteristic of R.A. in the cell and reference levels of expression of the genes. The set of reference levels of expression may include the levels of expression of the genes in a subject having R.A. The set of reference levels of expression may further include the levels of expression of the genes in a subject who does not have R.A. The method may comprise incubating a nucleic acid sample derived from the RNA of the cell of the subject with nucleic acids corresponding to the genes, under conditions wherein two complementary nucleic acids hybridize to each other. The nucleic acids corresponding to the genes may be attached to a solid surface. The method may comprise entering the levels of expression of the plurality of genes into a computer which comprises a memory with values representing the set of reference levels of expression. Comparing the level may comprise providing computer instructions to perform.

The invention provides methods for determining whether a subject has or is likely to develop R.A., comprising obtaining a cell from the subject and comparing gene expression levels in the cell to those of a set of reference levels of expression, e.g., as described above, wherein similar levels of expression of the plurality of genes indicates that the subject has or is likely to develop R.A. In a preferred embodiment, the cell is a peripheral blood mononuclear cell (PBMC) and the set of reference levels of expression includes the levels of expression of the genes in a PBMC of a subject having R.A. The cell may be a PBMC and the set of reference levels of expression includes the average of levels of expression of the genes in a PBMC of a plurality of subjects having R.A. The method may further comprising iteratively providing RNA from the subject and determining the level of RNA, such as to determine an evolution of the levels of expression of the genes in the subject.

Also within the scope of the invention are methods for determining whether a therapy for R.A. is effective in a subject having R.A. who is receiving the therapy. In a exemplary embodiment, the method comprises obtaining a cell from the subject and comparing levels of expression in the cell of the subject to those in subjects having R.A. and in subjects who do not have R.A., e.g., as described above, wherein levels of expression in the cell of the subject that are more similar to those of the subject having R.A. than the subject who does not have R.A. indicates that the therapy is not effective, whereas levels of expression in the cell of the subject that are more similar to those of the subject not having R.A. than the subject having R.A. indicates that the therapy is effective. The set of reference levels of expression may be in the form of a database. The database may be included in a computer-readable medium. The database may be in communications with a microprocessor and microprocessor instructions for providing a user interface to receive expression level data of a subject and to compare the expression level data with the database. In a particular embodiment, the method comprises (i) obtaining a patient sample from a caregiver; (ii) identifying expression levels of a plurality of genes characteristic of R.A. from the patient sample; (iii) determining whether the levels of expression of the genes in the patient sample are more similar to those of a subject having R.A. or to those of a subject who does not have R.A.; and (iv) transmitting the results to the caregiver. The results may be transmitted across a network.

In yet another embodiment, the invention provides methods for identifying a compound for treating R.A. The method comprises, e.g., (i) providing levels of expression of a plurality of genes characteristic of R.A. in a cell characteristic of R.A. incubated with a test compound; (ii) providing levels of expression of a normal counterpart cell; and (iii) comparing the two levels of expression, wherein similar levels of expression in the two cells indicates that the compound is likely to be effective for treating R.A.

Other methods provided by the invention include methods for selecting a therapy for a patient having R.A. For example, the method may comprise (i) providing at least one query value corresponding to the level of expression of at least one gene characteristic of R.A. from a patient having R.A.; (ii) providing a plurality of sets of reference values corresponding to levels of expression of at least one gene characteristic of R.A., each reference value being associated with a therapy; and (iii) selecting the reference values most similar to the query values, to thereby select a therapy for said patient. Selecting may further include weighing a comparison value for the reference values using a weight value associated with each reference values. The method may further comprise administering the therapy to the patient. The query values and the sets of reference values may be expression profiles. Another exemplary method comprises (i) providing a plurality of reference expression profiles, each associated with a therapy; (ii) providing a labeled target nucleic acid sample prepared from RNA of a diseased cell of the patient; (iii) contacting the labeled target nucleic acid sample with an array comprising probes corresponding to genes which are up- or down-regulated in R.A. to obtain an expression profile of the patient; and selecting the reference profile most similar to the expression profile of the patient, to thereby select a therapy for the patient.

The invention also provides therapeutic methods for treating R.A., including methods which normalize the expression level of one or more genes characteristic of R.A. in a subject diagnosed with R.A. “Normalization” of the level of expression of a gene refers to a change in the expression level of the gene such that its level of its expression resembles more that of a non-diseased (i.e., normal) cell than that of a diseased cell. Such methods may include administering to a subject having R.A. a phamarceutically efficient amount of an agonist or antagonist of one or more genes characteristic of R.A.

Also within the scope of the invention are diagnostic or drug discovery kits, comprising one or more computer-readable media, compositions and/or solid surfaces described herein, and optionally instructions for use.

### DETAILED DESCRIPTION OF THE INVENTION

The invention is based at least in part on the discovery of gene expression profiles of cells of subjects having R.A. As described in the Examples and in Tables 1-5, cells from R.A. subjects have genes which are expressed at higher levels (i.e., which are up-regulated) and genes which are expressed at lower levels (i.e., which are down-regulated) relative to cells of the same type in subjects which do not have any symptoms of R.A. In particular, as described in the Examples, it has been shown that genes SOCS3 (CISH3); RAGE (AGER); LST-1 (LY117); SAA 1-3; HMG-1; S100A8, A9, and A12; SLPI; GILZ; PTPN-18; GADD-45A and B; Legumain (PRSC1); FST1; Lcn2; GPI; SpiL; and TSG-6 are expressed at higher levels in the diseased cells relative to the corresponding normal cells. Other genes, e.g., CMAK2B; PLA2G2A; GBAS and SOX15, are down-regulated in the diseased cells relative to the corresponding normal cells.

1. Definitions:

As used herein, the following terms and phrases shall have the meanings set forth below. Unless defined otherwise, all technical and scientific terms used herein have the same meaning as commonly understood to one of ordinary skill in the art to which this invention belongs.

The singular forms “a,” “an,” and “the” include plural reference unless the context clearly dictates otherwise.

The phrase “a corresponding normal cell of” or “normal cell corresponding to” or “normal counterpart cell of” a diseased cell refers to a normal cell of the same type as that of the diseased cell. For example, a corresponding normal PBMC of a subject having R.A. is a PBMC of a subject not having R.A.

An “address” on an array, e.g., a microarray, refers to a location at which an element, e.g., an oligonucleotide, is attached to the solid surface of the array.

The term “agonist,” as used herein, is meant to refer to an agent that mimics or up-regulates (e.g., potentiates or supplements) the bioactivity of a protein. An agonist can be a wild-type protein or derivative thereof having at least one bioactivity of the wild-type protein. An agonist can also be a compound that upregulates expression of a gene or which increases at least one bioactivity of a protein. An agonist can also be a compound which increases the interaction of a polypeptide with another molecule, e.g., a target peptide or nucleic acid.

“Amplification,” as used herein, relates to the production of additional copies of a nucleic acid sequence. Amplification is generally carried out using polymerase chain reaction (PCR) technologies well known in the art. (Dieffenbach, C. W. and G. S. Dveksler (1995) PCR Primer, a Laboratory Manual, Cold Spring Harbor Press, Plainview, N.Y.)

“Antagonist” as used herein is meant to refer to an agent that downregulates (e.g., suppresses or inhibits) at least one bioactivity of a protein. An antagonist can be a compound which inhibits or decreases the interaction between a protein and another molecule, e.g., a target peptide or enzyme substrate. An antagonist can also be a compound that downregulates expression of a gene or which reduces the amount of expressed protein present.

The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Nonlimiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The subject invention includes polyclonal, monoclonal, or other purified preparations of antibodies and recombinant antibodies.

By “array” or “matrix” is meant an arrangement of addressable locations or “addresses” on a device. The locations can be arranged in two dimensional arrays, three dimensional arrays, or other matrix formats. The number of locations can range from several to at least hundreds of thousands. Most importantly, each location represents a totally independent reaction site. A “nucleic acid array” refers to an array containing nucleic acid probes, such as oligonucleotides or larger portions of genes. The nucleic acid on the array is preferably single stranded. Arrays wherein the probes are oligonucleotides are referred to as “oligonucleotide arrays” or “oligonucleotide chips.” A “microarray,” also referred to herein as a “biochip” or “biological chip” is an array of regions having a density of discrete regions of at least about 100/cm², and preferably at least about 1000/cm². The regions in a microarray have typical dimensions, e.g., diameters, in the range of between about 10-250 μm, and are separated from other regions in the array by about the same distance.

The term “biological sample”, as used herein, refers to a sample obtained from a subject, e.g., a human or from components (e.g., tissues) of a subject. The sample may be of any biological tissue or fluid. Frequently the sample will be a “clinical sample” which is a sample derived from a patient. Such samples include, but are not limited to, sputum, blood, blood cells (e.g., white cells), tissue or fine needle biopsy samples, urine, peritoneal fluid, and pleural fluid, or cells therefrom. Biological samples may also include sections of tissues such as frozen sections taken for histological purposes. A preferred biological sample is a PBMC sample or a sample from a joint, e.g., synovial fluid or synovial tissue.

The term “biomarker” of a disease refers to a gene which is up- or down-regulated in a diseased cell of a subject having R.A. relative to a counterpart normal cell, which gene is sufficiently specific to the diseased cell that it can be used, optionally with other genes, to identify or detect the disease. Generally, a biomarker is a gene that is characteristic of the disease.

A nucleotide sequence is “complementary” to another nucleotide sequence if each of the bases of the two sequences match, i.e., are capable of forming Watson-Crick base pairs. The term “complementary strand” is used herein interchangeably with the term “complement.” The complement of a nucleic acid strand can be the complement of a coding strand or the complement of a non-coding strand.

A “computer readable medium” is any medium that can be used to store data which can be accessed by a computer. Exemplary media include: magnetic storage media, such as a diskettes, hard drives, and magnetic tape; optical storage media such as CD-ROMs; electrical storage media such as RAM and ROM; and hybrids of these media, such as magnetic/optical storage medium.

A “cell characteristic of R.A.” refers to a cell present in subjects having R.A., which cell is a modified form of a normal cell and is not present in a subject not having R.A., or which cell is present in significantly higher or lower numbers in subjects having R.A. relative to subjects not having R.A. A “modified form of a normal cell” can be a form of the normal cell in which the expression of at least one gene is higher or lower (e.g., by 50%, 2 fold, 5 fold, or over 10 fold) relative to the normal cell. A cell characteristic of R.A. is also referred to herein as a “diseased cell of R.A.” Exemplary diseased cells of R.A. include PBMCs, e.g., monocytes and macrophages, and inflammatory cells present in joints of patients, in particular, in synovial fluid and synovium. Inflammatory cells can be lymphocytes, e.g., T lymphocytes, B lymphocytes, monocytes and macrophages. Other diseased cells of R.A. include neutrophils, fibroblasts, endothelial cells, osteoclasts, osteoblasts, osteocytes, chondrocytes, and cells present in cartilage.

A “cell corresponding to a cell characteristic of R.A.” refers to a cell which has essentially the same phenotype as that of a cell characteristic of R.A. For example, a cell corresponding to a PBMC or a subject having R.A. is a PBMC of a subject who does not have R.A.

A “cell sample characteristic of R.A.” or a “tissue sample characteristic of R.A.” refers to a sample of cells, such as a tissue, that contains at least one cell characteristic of R.A. Such a sample may be a sample of blood, PBMCs, synovial fluid, synovium, cartilage or bone.

The term “derivative” refers to the chemical modification of a compound, e.g., a polypeptide, or a polynucleotide. Chemical modifications of a polynucleotide can include, for example, replacement of hydrogen by an alkyl, acyl, or amino group. A derivative polynucleotide encodes a polypeptide which retains at least one biological or immunological function of the natural molecule. A derivative polypeptide can be one modified by glycosylation, pegylation, or any similar process that retains at least one biological or immunological function of the polypeptide from which it was derived.

A “detection agent of a gene” refers to an agent that can be used to specifically detect a gene or other biological molecule relating to it, e.g., RNA transcribed from the gene and polypeptides encoded by the gene. Exemplary detection agents are nucleic acid probes which hybridize to nucleic acids corresponding to the gene and antibodies.

The term “equivalent” is understood to include nucleotide sequences encoding functionally equivalent polypeptides. Equivalent nucleotide sequences will include sequences that differ by one or more nucleotide substitutions, additions or deletions, such as allelic variants; and will, therefore, include sequences that differ from the nucleotide sequence of the nucleic acids referred to in Any of Tables 1-5 due to the degeneracy of the genetic code.

The term “essentially all the genes of any of Tables 1-5” refers to at least 90%, preferably at least 95% and most preferably at least 98% of the genes of any of Tables 1-5.

The term “expression profile,” which is used interchangeably herein with “gene expression profile” and “finger print” refers to a set of values representing the activity of about 10 or more genes. An expression profile preferably comprises values representing expression levels of at least about 20 genes, preferably at least about 30, 50, 100, 200 or more genes. An expression profile can be a set of values obtained from one or more cells or from a tissue sample, e.g., a clinical sample. An expression profile of a cell characteristic of R.A. may refer to a set of values representing mRNA levels of about 10 or more genes in a cell characteristic of R.A. An “expression profile of R.A.” refers to an expression profile of a cell characteristic of R.A. Thus, since there are different cells characteristic of R.A., there may be different expression profiles of R.A.

“Genes which are up- or down-regulated in R.A.” refers to genes which are up- or down-regulated in cells characteristic of R.A. relative to normal counterpart cells.

“Genes characteristic of R.A.” refers to genes which are up- or down-regulated by a significant factor, e.g., at least about 1.1 fold, 1.25 fold, 1.5 fold, 2 fold, 5 fold, 10 fold or more in at least about 50%, preferably 60%, 70%, 80%, or 90% of subjects having R.A., as determined, e.g., by methods described herein. Preferred genes characteristic of R.A. are those described in Tables 1-5. Even more preferred genes are those which are highlighted or marked with a star in the Tables, those which encode kinases or phosphatases and those wich are located on human chromosome 6, preferably at 6p21.3.

“Hybridization” refers to any process by which a strand of nucleic acid binds with a complementary strand through base pairing. Two single-stranded nucleic acids “hybridize” when they form a double-stranded duplex. The region of double-strandedness can include the full-length of one or both of the single-stranded nucleic acids, or all of one single stranded nucleic acid and a subsequence of the other single stranded nucleic acid, or the region of double-strandedness can include a subsequence of each nucleic acid. Hybridization also includes the formation of duplexes which contain certain mismatches, provided that the two strands are still forming a double stranded helix. “Stringent hybridization conditions” refers to hybridization conditions resulting in essentially specific hybridization.

The term “isolated” as used herein with respect to nucleic acids, such as DNA or RNA, refers to molecules separated from other DNAs, or RNAs, respectively, that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

As used herein, the terms “label” and “detectable label” refer to a molecule capable of detection, including, but not limited to, radioactive isotopes, fluorophores, chemiluminescent moieties, enzymes, enzyme substrates, enzyme cofactors, enzyme inhibitors, dyes, metal ions, ligands (e.g., biotin or haptens) and the like. The term “fluorescer” refers to a substance or a portion thereof which is capable of exhibiting fluorescence in the detectable range. Particular examples of labels which may be used under the invention include fluorescein, rhodamine, dansyl, umbelliferone, Texas red, luminol, NADPH, alpha - beta -galactosidase and horseradish peroxidase.

The “level of expression of a gene in a cell” refers to the activity of a gene in the cell, which can be indicated by the level of mRNA, as well as pre-mRNA nascent transcript(s), transcript processing intermediates, mature mRNA(s) and degradation products, encoded by the gene in the cell.

The phrase “normalizing expression of a gene” in a diseased cell refers to an action to compensate for the altered expression of the gene in the diseased cell, so that it is essentially expressed at the same level as in the corresponding non diseased cell. For example, where the gene is over-expressed in the diseased cell, normalization of its expression in the diseased cell refers to treating the diseased cell in such a way that its expression becomes essentially the same as the expression in the counterpart normal cell. “Normalization” preferably brings the level of expression to within approximately a 50% difference in expression, more preferably to within approximately a 25%, and even more preferably 10% difference in expression. The required level of closeness in expression will depend on the particular gene, and can be determined as described herein. The phrase “normalizing gene expression in a diseased cell” refers to an action to normalize the expression of essentially all genes in the diseased cell.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

The phrase “nucleic acid corresponding to a gene” refers to a nucleic acid that can be used for detecting the gene, e.g., a nucleic acid which is capable of hybridizing specifically to the gene.

The phrase “nucleic acid sample derived from RNA” refers to one or more nucleic acid molecule, e.g., RNA or DNA, that was synthesized from the RNA, and includes DNA resulting from methods using PCR, e.g., RT-PCR.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases. Databases with individual sequences are described in Methods in Enzymology, ed. Doolittle, supra. Databases include Genbank, EMBL, and DNA Database of Japan (DDBJ).

“Perfectly matched” in reference to a duplex means that the poly- or oligonucleotide strands making up the duplex form a double stranded structure with one other such that every nucleotide in each strand undergoes Watson-Crick basepairing with a nucleotide in the other strand. The term also comprehends the pairing of nucleoside analogs, such as deoxyinosine, nucleosides with 2-aminopurine bases, and the like, that may be employed. A mismatch in a duplex between a target polynucleotide and an oligonucleotide or olynucleotide means that a pair of nucleotides in the duplex fails to undergo Watson-Crick bonding. In reference to a triplex, the term means that the triplex consists of a perfectly matched duplex and a third strand in which every nucleotide undergoes Hoogsteen or reverse Hoogsteen association with a basepair of the perfectly matched duplex.

A “plurality” refers to two or more.

As used herein, a nucleic acid or other molecule attached to an array, is referred to as a “probe” or “capture probe.” When an array contains several probes corresponding to one gene, these probes are referred to as “gene-probe set.” A gene-probe set can consist of, e.g., 2 to 10 probes, preferably from 2 to 5 probes and most preferably about 5 probes.

The “profile” of a cell's biological state refers to the levels of various constituents of a cell that are known to change in response to drug treatments and other perturbations of the cell's biological state. Constituents of a cell include levels of RNA, levels of protein abundances, or protein activity levels.

The term “protein” is used interchangeably herein with the terms “peptide” and “polypeptide.”

“Rheumatoid arthritis” or “R.A.” refers to a systemic chronic inflammatory disease involving primarily the joints of the extremities. It is characterized by destruction of the joint cartilage and inflammation of the synovium, with a morphologic picture suggestive of a local immune response. CD4⁺ T cells, activated B lymphocytes and plasma cells are found in the inflamed synovium, and in severe cases, well formed lymphoid follicles with germinal centers may be present. The synovial fluid and serum contain rheumatoid factors, i.e., complexes containing auto-antibodies, and may cytokines, e.g., interleukin-1 (IL-1), tumor necrosis factor (TNF) and interferon gamma (IFN-γ). T cells expressing the γδ antigen receptor are also present in the synovial fluid of patients. R.A. is described, e.g., in Cecil Essentials of Medicine, Third Edition, Andreoli et al., W.B. Saunders Company (1993) at pages 564 to 568. This reference describes in particular symptoms that are the basis of a diagnosis of R.A. This reference also describes the different stages of the disease. Briefly, the first stage is characterized by presentation of antigen to T cells and is not associated with any symptoms. The second stage is characterized by T- and B-cell proliferation and angiogenesis in synovial membrane. The symptoms of the second stage are malaise, mild joint stiffness and swelling. The third stage is characterized by accumulation of neutrophils in synovial fluid; synovial cell proliferation without polarization or invasion or cartilage. The symptoms in this stage are joint pain and swelling; morning stiffness, malaise and weakness. The fourth stage is characterized by polarization of synovitis into a centripetally invasive pannus; activation of chondrocytes; initiation of enzyme (proteinase) degradation of cartilage. The symptoms in this stage are the same as those associated with stage three. The fifth stage is characterized by erosion of subchondral bone; invasion of cartilage by pannus; chrondrocyte proliferation; and stretched ligaments around joints. The symptoms in this stage are the same as those associated with stage 3, and in addition, loss of function and early deformity (e.g., ulnar deviation at metacarpophalangeal joint). Therapeutics used for treating R.A. include aspirin or other non-steroidal anti-inflammatory drug (NSAID); immunosuppressive agents, e.g., azathioprine, cyclophosphamide, chlorambucil and methotrexate; corticosteroids; gold salts; penicillamine; Infliximab™ (anti-TNF antibody); Etanercept™ or Enbrel™ (a soluble TNF receptor); Leflunomide™; Anakinra (IL-I antagonist); and Kinaret™ (IL-I antagonist).

A “similarity” between the level of expression of a gene in two cells or tissues refers to a difference in expression levels of a factor of at least about 10% (i.e., 1.1 fold), 25% (i.e., 1.25 fold), 50% (i.e., 1.5 fold), 75% (i.e., 1.75 fold), 90% (i.e., 1.9 fold), 2 fold, 2.5 fold, 3 fold, 5 fold, 10 fold, 50 fold, or 100 fold. Expression levels can be raw data or they can averaged or normalized data, e.g., normalized relative to normal controls.

An expression profile in one cell or tissue is “similar” to an expression profile in another cell or tissue when the level of expression of the genes in the two expression profiles are sufficiently similar that the similarity is indicative of a common characteristic, e.g., being of the same cell type, or being characteristic of R.A. “Similarity” between an expression profile of a cell or tissue, e.g., of a subject, and a set of data representing an expression profile characteristic of a disease can be based on the presence or absence in the cell or tissue of certain RNAs and/or certain levels of certain RNAs of genes having a high probability of being associated with the disease. A high probability of being associated with a disease can be, e.g., the presence of RNA or of certain levels of RNA of particular genes which are over-expressed or under-expressed, in at least about 50%, 60%, 70%, 80%, 90%, or 100% of patients having the disease. A similarity -with an expression profile of a patient can also be based on higher or lower expression levels of a factor of at least about 10%, 25%, 50%, 75%, 1.5 fold, 2 fold, 2.5 fold, 3 fold, 5 fold, 10 fold, 50 fold, 100 fold of at least about 50%, 60%, 70%, 80%, 90%, or 100% of genes, or at least about 10, 50, 100, 200, 300 genes, which are up- or down-regulated in at least about 50%, 60%, 70%, 80%, 90%, or 100% of patients. For example, the expression profile of PBMCs of a subject is similar to a reference expression profile fo an R.A. patient, e.g., determined herein, if at least about 50 genes which are over-expressed or repressed (i.e., under-expressed), e.g., at least about 1.1 fold, in at least about 60% of the patients studied are over-expressed or repressed, e.g., about at least 1.1 fold, in the expression profile of the subject. A similarity in expression profiles may also include similar expression levels of genes which are not up- or down-regulated in R.A.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon-containing) or inorganic molecules. Many pharmaceutical companies have extensive libraries of chemical and/or biological mixtures, often fungal, bacterial, or algal extracts, which can be screened with any of the assays of the invention to identify compounds that modulate a bioactivity.

The term “specific hybridization” of a probe to a target site of a template nucleic acid refers to hybridization of the probe predominantly to the target, such that the hybridization signal can be clearly interpreted. As further described herein, such conditions resulting in specific hybridization vary depending on the length of the region of homology, the GC content of the region, the melting temperature “Tm” of the hybrid. Hybridization conditions will thus vary in the salt content, acidity, and temperature of the hybridization solution and the washes.

A “subject” can be a mammal, e.g., a human, primate, ovine, bovine, porcine, equine, feline, and canine.

The term “treating” a disease in a subject or “treating” a subject having a disease refers to providing the subject with a pharmaceutical treatment, e.g., the administration of a drug, such that at least one symptom of the disease is decreased. Treating a disease can be preventing the disease, improving the disease or curing the disease. Treatment of R.A. includes inhibition of erosion, e.g., cartilage or bone erosion, and/or inhibition of inflammation.

The phrase “value representing the level of expression of a gene” refers to a raw number which reflects the mRNA level of a particular gene in a cell or biological sample, e.g., obtained from analytical tools for measuring RNA levels.

A “variant” of a polypeptide refers to a polypeptide having the amino acid sequence of the polypeptide, in which one or more amino acid residues are altered. The variant may have “conservative” changes, wherein a substituted amino acid has similar structural or chemical properties (e.g., replacement of leucine with isoleucine). More rarely, a variant may have “non-conservative” changes (e.g., replacement of glycine with tryptophan). Analogous minor variations may also include amino acid deletions or insertions, or both. Guidance in determining which amino acid residues may be substituted, inserted, or deleted without abolishing biological or immunological activity may be found using computer programs well known in the art, for example, LASERGENE software (DNASTAR). The term “variant,” when used in the context of a polynucleotide sequence, encompasses a polynucleotide sequence related to that of a gene of interest or the coding sequence thereof. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one base. The presence of SNPs may be indicative of, for example, a certain population, a disease state, or a propensity for a disease state.

2. R.A. Diagnostic and Prognostic Methods of Use

The invention provides gene expression profiles of R.A. As further described herein, the gene expression profiles of the diseased cells of subjects having R.A., indicates that genes certain genes, e.g., SOCS3 (CISH3); RAGE (AGER); LST-1 (LY117); SAA 1-3; HMG-1; S100 A8, A9, and A12; SLPI; GILZ; PTPN-18; GADD-45A and B; Legumain (PRSC1); FST1; Lcn2; GPI; SpiL; and TSG-6, are significantly up-regulated in these cells relative to their normal counterparts. The expression data also show that certain genes, e.g., CAMK2B, PLA2G2A, GBAS and SOX15, are significantly down-regulated in the diseased cells relative to their normal counterpart cells. Other preferred genes include those that are highlighted or marked with a star in Tables 1-5. Yet other genes of particular interest are those that have a fold induction indicated as “#DIV/0!” in the Tables; those that encode kinases and phosphatases; those that are localized to human chromosome 6p21.3; and those which are highlighted or marked with a star in the Tables. Accordingly, the expression profile can be used diagnostically and prognostically for R.A. Exemplary diagnostic tools and assays are set forth below, under (i) to (vi), followed by exemplary methods for conducting these assays.

Preferred methods of the invention involve measuring the level of expression of one or more genes that are up- or down-regulated in R.A. in a cell of a patient, and comparing these levels of expression to the level of expression of the genes in other samples, which levels of expression may be present in a computer readable medium and analyzed with a computer.

(i) In one embodiment, the invention provides a method for determining whether a subject has or is likely to develop R.A., comprising determining the level of expression of one or more genes which are up- or down-regulated in R.A. in a cell of the subject and comparing these levels of expression with the levels of expression of the genes in a diseased cell of a subject known to have R.A. A similar level of expression of the genes in the two cells is indicative that the subject has or is likely to develop R.A. or at least a symptom thereof. In a preferred embodiment, the cell of the subject is essentially of the same type as that which is diseased in R.A.

(ii) In another embodiment the expression profile data of the invention can be used to confirm that a subject has R.A., and in particular, that the subject does not have a disease that is merely related R.A. This can be important, in particular, in designing an optimal therapeutic regimen for the subject. It has been described in the art that expression profiles can be used to distinguish one type of disease from a similar disease. For example, two subtypes of non-Hodgkin's lymphomas, one of which responds to current therapeutic methods and the other one which does not, could be differentiated by investigating 17,856 genes in specimens of patients suffering from diffuse large B-cell lymphoma (Alizadeh et al. (2000) Nature 405:503). Similarly, subtypes of cutaneous melanoma were predicted based on profiling 8150 genes (Bittner et al. (2000) Nature 406:536). In this case, features of the highly aggressive metastatic melanomas could be recognized. Numerous other studies comparing expression profiles of cancer cells and normal cells have been described, including studies describing expression profiles distinguishing between highly and less metastatic cancers and studies describing new subtypes of diseases, e.g., new tumor types (see, e.g., Perou et al. (1999) PNAS 96: 9212; Perou et al. (2000) Nature 606:747; Clark et al. (2000) Nature 406:532; Alon et al. (1999) PNAS 96:6745; Golub et al. (1999) Science 286:531).

Accordingly, the expression profiles of the invention allow the distinction of R.A. from related diseases. In a preferred embodiment, the level of expression of one or more genes which are up- or down-regulated in R.A. is determined in a cell of the subject, preferably a cell which corresponds to a diseased cell in R.A. A level of expression of one or more genes that is more similar to that in a cell characteristic of R.A. than to that of cells of related diseases indicates that the subject has R.A., rather than a disease related to R.A.

Prior to using this method for determining whether the subject has R.A. or a related disease, it may be necessary to first determine the expression profile of cells of diseases that are similar to R.A. This can be undertaken using the same microarray as the one that was used to identify the genes characteristic of R.A., and according to methods further described herein.

(iii) In yet another embodiment, the invention provides methods for determining the stage of R.A. in the subject. In one embodiment, the level of expression of one or more genes that are up- or down-regulated in R.A., in particular, whose level of expression varies with the stage of the disease is determined in a cell of a subject. A level of expression of one or more genes that is more similar to that of one stage of the disease (stage “a”) relative to that in other stages of the disease indicates that the disease of the subject is in stage a.

This assay may require the preliminary determination of expression profiles in different stages of R.A. Such expression data can be obtained by, e.g., using microarrays with target nucleic acids made from RNA of patients at different stages of the disease.

(iv) The method can also be used to determine the efficacy of a therapy in a subject. Accordingly, in one embodiment, the level of expression of one or more genes which are up- or down-regulated in R.A. is determined in a subject before the treatment and one or more times during the treatment. For example, a sample of RNA can be obtained from the subject before the beginning of the therapy and every 12, 24 or 72 hours during the therapy. Samples can also be analyzed once a week or once a month. Changes in expression levels of the genes over time and relative to diseased cells and normal cells will indicate whether the therapy is effective. For example, expression levels that are more similar to those in normal cells or in less advanced stages of the disease relative to the stage the subject was in, indicates that the therapy is effective.

(v) In yet another embodiment, the invention provides a method for determining the likelihood of success of a particular therapy in a subject having R.A. In one embodiment, a subject is started on a particular therapy, and the effectiveness of the therapy is determined, e.g., by determining the level of expression of one or more genes characteristic of R.A. in a cell of the subject. A normalization of the level of expression of these genes, i.e., a change in the expression level of the genes such that their level of expression resembles more that of a non diseased cell, indicates that the treatment should be effective in the subject. On the other hand, the absence of normalization of the level of expression of the genes characteristic of R.A. indicates that the treatment is not likely to be effective in the subject. This method may be able to predict that a treatment is effective before any alleviation of symptoms becomes apparent.

Prediction of the outcome of a treatment of R.A. in a subject can also be undertaken in vitro. In one embodiment, cells are obtained from a subject to be evaluated for responsiveness to the treatment, and incubated in vitro with the therapeutic drug or metabolized form thereof. The level of expression of one or more genes which are up- or down-regulated in R.A. is measured in the cells and these values are compared to the level of expression of these one or more genes in a cell which is a normal counterpart cell of a cell characteristic of R.A. The level of expression can also be compared to that in other diseased cells. A level of expression of one or more genes in the cells of the subject after incubation with the drug that is similar to their level of expression in a normal cell and different from that in a diseased cell is indicative that it is likely that the subject will respond positively to a treatment with the drug. On the contrary, levels of expressions that are more similar to levels of expression in a diseased cell than that in a normal cell is indicative that it is likely that the subject will not respond positively to a treatment with the drug.

Since it is possible that a drug for treating R.A. does not act directly on the diseased cells, but is, e.g., metabolized, or acts on another cell which then secretes a factor that will effect the diseased cells, the above assay can also be conducted in a tissue sample of a subject, which contains cells other than the diseased cells. For example, a tissue sample comprising diseased cells is obtained from a subject; the tissue sample is incubated with the potential drug; optionally one or more diseased cells are isolated from the tissue sample, e.g., by microdissection or Laser Capture Microdissection (LCM, see infra); and the expression level of one or more genes characteristic of R.A. is examined.

(vi) The invention also provides methods for selecting a particular therapy for an R.A. patient from a selection of several different therapies. Certain subjects having R.A. may respond better to one type of therapy than to another type of therapy. In a preferred embodiment, the method comprises comparing the expression level of at least one gene that is up- or down-regulated in R.A. in the patient with that in cells of R.A. subjects that were treated in vitro or in vivo with one of several therapeutic drugs, which subjects are responders or non responders to one of the therapeutic drugs, and identifying the cell which has the most similar level of expression of that in the patient, to thereby identify a therapy for the patient. The method may further comprise administering the therapy to the subject.

A person of skill in the art will recognize that in certain diagnostic and prognostic assays, it will be sufficient to assess the level of expression of a single gene that is up- or down-regulated in R.A., and that in others, the expression of a plurality, e.g., two or more genes, is preferred. In certain embodiments, it is preferable to assess the expression of at least about 10%, or at least about 20%, 30%, 50%, 70%, 90% or 95% of the genes listed in one or more of Tables 1-5 or of the genes characteristic of R.A.

A person of skill in the art will also recognize that expression levels can be measured in a single cell or in a plurality of cells, e.g., two or more cells. In one embodiment, the method comprises determined expression levels in a cell or tissue sample, e.g., a blood sample, a PBMC sample, a synovial fluid sample or a synovium sample.

Set forth below are exemplary methods which can be used to determine the level of expression of one or more genes. In a preferred embodiment for determining the level of expression of a plurality of genes, arrays, e.g., microarrays, can be used.

2.1. Use of Arrays for Determining the Level of Expression of Genes

Generally, determining expression profiles with arrays involves the following steps: (a) obtaining a mRNA sample from a subject and preparing labeled nucleic acids therefrom (the “target nucleic acids” or “targets”); (b) contacting the target nucleic acids with the array under conditions sufficient for target nucleic acids to bind with corresponding probes on the array, e.g. by hybridization or specific binding; (c) optionally removing unbound targets from the array; (d) detecting bound targets, and (e) analyzing the results. As used herein, “nucleic acid probes” or “probes” are nucleic acids attached to the array, whereas “target nucleic acids” are nucleic acids that are hybridized to the array. Each of these steps is described in more detail below.

(i) Obtaining a mRNA Sample of a Subject

In one embodiment, one or more cells from the subject to be tested are obtained and RNA is isolated from the cells. In a preferred embodiment, PBMCs, synovial fluid, synovium or cartilage are obtained from the subject according to methods known in the art. Examples of such methods are set forth in the Examples and is discussed by Kim, C. H. et al. (J. Virol. 66:3879-3882 (1992)); Biswas, B. et al. (Annals NY Acad. Sci. 590:582-583 (1990)); Biswas, B. et al. (J. Clin. Microbiol. 29:2228-2233 (1991)). When obtaining the cells, it is preferable to obtain a sample containing predominantly cells of the desired type, e.g., a sample of cells in which at least about 50%, preferably at least about 60%, even more preferably at least about 70%, 80% and even more preferably, at least about 90% of the cells are of the desired type. A higher percentage of cells of the desired type is preferable, since such a sample is more likely to provide clear gene expression data.

It is also possible to obtain a cell sample from a subject, and then to enrich it for a desired cell type. For example, PBMCs can be isolated from blood as described herein. Counter-flow centrifugation (elutriation) can also be used to enrich for various cell types, such as T cells, B cells and monocytes, from PBMCs. Cells can also be isolated from other cells using a variety of techniques, such as isolation with an antibody binding to an epitope on the cell surface of the desired cell type. Another method that can be used includes negative selection using antibodies to cell surface markers to selectively enrich for a specific cell type without activating the cell by receptor engagement. Where the desired cells are in a solid tissue, particular cells can be dissected out, e.g., by microdissection. Exemplary cells that one may want to enrich for include monocytes, macrophages, T and B cells, osteocytes, osteoblasts, osteoclasts, chondrocytes, fibroblasts, neutrophils, endothelial cells and other cartilage cells.

In one embodiment, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated (see, e.g., Bonner et al. (1997) Science 278: 1481; Emmert-Buck et al. (1996) Science 274:998; Fend et al. (1999) Am. J. Path. 154: 61 and Murakami et al. (2000) Kidney Int. 58:1346). For example, Murakami et al., supra, describe isolation of a cell from a previously immunostained tissue section.

It is also be possible to obtain cells from a subject and culture the cells in vitro, such as to obtain a larger population of cells from which RNA can be extracted. Methods for establishing cultures of non-transformed cells, i.e., primary cell cultures, are known in the art.

When isolating RNA from tissue samples or cells from individuals, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells may quickly become degraded. Accordingly, in a preferred embodiment, the tissue or cells obtained from a subject is snap frozen as soon as possible.

RNA can be extracted from the tissue sample by a variety of methods, e.g., those described in the Examples or guanidium thiocyanate lysis followed by CsCl centrifugation (Chirgwin et al., 1979, Biochemistry 18:5294-5299). RNA from single cells can be obtained as described in methods for preparing cDNA libraries from single cells, such as those described in Dulac, C. (1998) Curr. Top. Dev. Biol. 36, 245 and Jena et al. (1996) J. Immunol. Methods 190:199. Care to avoid RNA degradation must be taken, e.g., by inclusion of RNAsin.

The RNA sample can then be enriched in particular species. In one embodiment, poly(A)+ RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.).

In a preferred embodiment, the RNA population is enriched in sequences of interest, such as those of genes characteristic of R.A. Enrichment can be undertaken, e.g., by primer-specific cDNA synthesis, or multiple rounds of linear amplification based on cDNA synthesis and template-directed in vitro transcription (see, e.g., Wang et al. (1989) PNAS 86, 9717; Dulac et al., supra, and Jena et al., supra).

The population of RNA, enriched or not in particular species or sequences, can further be amplified. Such amplification is particularly important when using RNA from a single or a few cells. A variety of amplification methods are suitable for use in the methods of the invention, including, e.g., PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. USA, 87, 1874 (1990)); nucleic acid based sequence amplification (NASBA) and transcription amplification (see, e.g. Kwoh et al., Proc. Natl. Acad. Sci. USA 86, 1173 (1989)). For PCR technology, see, e.g., PCR Technology: Principles and Applications for DNA Amplification (ed. H. A. Erlich, Freeman Press, N.Y., N.Y., 1992); PCR Protocols: A Guide to Methods and applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202. Methods of amplification are described, e.g., in Ohyama et al. (2000) BioTechniques 29:530; Luo et al. (1999) Nat. Med. 5, 117; Hegde et al. (2000) BioTechniques 29:548; Kacharmina et al. (1999) Meth. Enzymol. 303:3; Livesey et al. (2000) Curr. Biol. 10:301; Spirin et al. (1999) Invest. Ophtalmol. Vis. Sci. 40:3108; and Sakai et al. (2000) Anal. Biochem. 287:32. RNA amplification and cDNA synthesis can also be conducted in cells in situ (see, e.g., Eberwine et al. (1992) PNAS 89:3010).

One of skill in the art will appreciate that whatever amplification method is used, if a quantitative result is desired, care must be taken to use a method that maintains or controls for the relative frequencies of the amplified nucleic acids to achieve quantitative amplification. Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. A high density array may then include probes specific to the internal standard for quantification of the amplified nucleic acid.

One preferred internal standard is a synthetic AW106 cRNA. The AW106 ERNA is combined with RNA isolated from the sample according to standard techniques known to those of skilled in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of radioactivity (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al., Academic Press, Inc. N.Y., (1990).

In a preferred embodiment, a sample mRNA is reverse transcribed with a reverse transcriptase and a primer consisting of oligo(dT) and a sequence encoding the phage T7 promoter to provide single stranded DNA template. The second DNA strand is polymerized using a DNA polymerase. After synthesis of double-stranded cDNA, T7 RNA polymerase is added and RNA is transcribed from the cDNA template. Successive rounds of transcription from each single cDNA template results in amplified RNA. Methods of in vitro polymerization are well known to those of skill in the art (see, e.g., Sambrook, (supra) and this particular method is described in detail by Van Gelder, et al., Proc. Natl. Acad. Sci. USA, 87: 1663-1667 (1990) who demonstrate that in vitro amplification according to this method preserves the relative frequencies of the various RNA transcripts). Moreover, Eberwine et al. Proc. Natl. Acad. Sci. USA, 89: 3010-3014 provide a protocol that uses two rounds of amplification via in vitro transcription to achieve greater than 106 fold amplification of the original starting material, thereby permitting expression monitoring even where biological samples are limited.

It will be appreciated by one of skill in the art that the direct transcription method described above provides an antisense (aRNA) pool. Where antisense RNA is used as the target nucleic acid, the oligonucleotide probes provided in the array are chosen to be complementary to subsequences of the antisense nucleic acids. Conversely, where the target nucleic acid pool is a pool of sense nucleic acids, the oligonucleotide probes are selected to be complementary to subsequences of the sense nucleic acids. Finally, where the nucleic acid pool is double stranded, the probes may be of either sense as the target nucleic acids include both sense and antisense strands.

(ii) Labeling of the Nucleic Acids to be Analyzed

Generally, the target molecules will be labeled to permit detection of hybridization of target molecules to a microarray. By “labeled” is meant that the probe comprises a member of a signal producing system and is thus detectable, either directly or through combined action with one or more additional members of a signal producing system. Examples of directly detectable labels include isotopic and fluorescent moieties incorporated into, usually covalently bonded to, a moiety of the probe, such as a nucleotide monomeric unit, e.g. dNMP of the primer, or a photoactive or chemically active derivative of a detectable label which can be bound to a functional moiety of the probe molecule.

Nucleic acids can be labeled after or during enrichment and/or amplification of RNAs. For example, labeled cDNA can be prepared from mRNA by oligo dT-primed or random-primed reverse transcription, both of which are well known in the art (see, e.g., Klug and Berger, 1987, Methods Enzymol. 152:316-325). Reverse transcription may be carried out in the presence of a dNTP conjugated to a detectable label, most preferably a fluorescently labeled dNTP. Alternatively, isolated mRNA can be converted to labeled antisense RNA synthesized by in vitro transcription of double-stranded cDNA in the presence of labeled dNTPs (Lockhart et al., 1996, Expression monitoring by hybridization to high-density oligonucleotide arrays, Nature Biotech. 14:1675). In alternative embodiments, the cDNA or RNA probe can be synthesized in the absence of detectable label and may be labeled subsequently, e.g., by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent.

In one embodiment, labeled cDNA is synthesized by incubating a mixture containing RNA and 0.5 mM dGTP, dATP and dCTP plus 0.1 mM dTTP plus fluorescent deoxyribonucleotides (e.g., 0.1 mM Rhodamine 110 UTP (Perken Elmer Cetus) or 0.1 mM Cy3 dUTP (Amersham)) with reverse transcriptase (e.g., SuperScript.™.II, LTI Inc.) at 42° C. for 60 mm.

Fluorescent moieties or labels of interest include coumarin and its derivatives, e.g. 7-amino-4-methylcoumarin, aminocoumarin, bodipy dyes, such as Bodipy Fla., cascade blue, fluorescein and its derivatives, e.g. fluorescein isothiocyanate, Oregon green, rhodamine dyes, e.g. Texas red, tetramethylrhodamine, eosins and erythrosins, cyanine dyes, e.g. Cy2, Cy3, Cy3.5, Cy5, Cy5.5, Cy7, FluorX, macrocyclic chelates of lanthanide ions, e.g. quantum dye™, fluorescent energy transfer dyes, such as thiazole orange-ethidium heterodimer, TOTAB, dansyl, etc. Individual fluorescent compounds which have functionalities for linking to an element desirably detected in an apparatus or assay of the invention, or which can be modified to incorporate such functionalities include, e.g., dansyl chloride; fluoresceins such as 3,6-dihydroxy-9-phenylxanthydrol; rhodamineisothiocyanate; N-phenyl 1-amino-8-sulfonatonaphthalene; N-phenyl 2-amino-6-sulfonatonaphthalene; 4-acetamido-4-isothiocyanatostilbene-2,2′-disulfonic acid; pyrene-3-sulfonic acid; 2-toluidinonaphthalene-6-sulfonate; N-phenyl-N-methyl-2-aminoaphthalene-6-sulfonate; ethidium bromide; stebrine; auromine-0,2-(9′-anthroyl)palmitate; dansyl phosphatidylethanolamine; N,N′-dioctadecyl oxacarbocyanine: N,N′-dihexyl oxacarbocyanine; merocyanine, 4-(3′-pyrenyl)stearate; d-3-aminodesoxy-equilenin; 12-(9′-anthroyl)stearate; 2-methylanthracene; 9-vinylanthracene; 2,2′(vinylene-p-phenylene)bisbenzoxazole; p-bis(2-methyl-5-phenyl-oxazolyl))benzene; 6-dimethylamino-1,2-benzophenazin; retinol; bis(3′-aminopyridinium) 1,10-decandiyl diiodide; sulfonaphthylhydrazone of hellibrienin; chlorotetracycline; N-(7-dimethylamino-4-methyl-2-oxo-3-chromenyl)maleimide; N-(p-(2benzimidazolyl)-phenyl)maleimide; N-(4-fluoranthyl)maleimide; bis(homovanillic acid); resazarin; 4-chloro-7-nitro-2,1,3-benzooxadiazole; merocyanine 540; resorufin; rose bengal; and 2,4-diphenyl-3(2H)-furanone. (see, e.g., Kricka, 1992, Nonisotopic DNA Probe Techniques, Academic Press San Diego, Calif.). Many fluorescent tags are commercially available from SIGMA chemical company (Saint Louis, Mo.), Amersham, Molecular Probes, R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), and Applied Biosystems (Foster City, Calif.) as well as other commercial sources known to one of skill.

Chemiluminescent labels include luciferin and 2,3-dihydrophthalazinediones, e.g., luminol.

Isotopic moieties or labels of interest include ³²P, ³³P, ³⁵S, ¹²⁵I,²H, ¹⁴C, and the like (see Zhao et al., 1995, High density cDNA filter analysis: a novel approach for large-scale, quantitative analysis of gene expression, Gene 156:207; Pietu et al., 1996, Novel gene transcripts preferentially expressed in human muscles revealed by quantitative hybridization of a high density cDNA array, Genome Res. 6:492).

Labels may also be members of a signal producing system that act in concert with one or more additional members of the same system to provide a detectable signal. Illustrative of such labels are members of a specific binding pair, such as ligands, e.g. biotin, fluorescein, digoxigenin, antigen, polyvalent cations, chelator groups and the like, where the members specifically bind to additional members of the signal producing system, where the additional members provide a detectable signal either directly or indirectly, e.g. antibody conjugated to a fluorescent moiety or an enzymatic moiety capable of converting a substrate to a chromogenic product, e.g. alkaline phosphatase conjugate antibody and the like.

Additional labels of interest include those that provide for signal only when the probe with which they are associated is specifically bound to a target molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037; WO 97/17471 and WO 97/17076.

In some cases, hybridized target nucleic acids may be labeled following hybridization. For example, where biotin labeled dNTPs are used in, e.g., amplification or transcription, streptavidin linked reporter groups may be used to label hybridized complexes.

In other embodiments, the target nucleic acid is not labeled. In this case, hybridization can be determined, e.g., by plasmon resonance, as described, e.g., in Thiel et al. (1997) Anal. Chem. 69:4948.

In one embodiment, a plurality (e.g., 2, 3, 4, 5 or more) of sets of target nucleic acids are labeled and used in one hybridization reaction (“multiplex” analysis). For example, one set of nucleic acids may correspond to RNA from one cell or tissue sample and another set of nucleic acids may correspond to RNA from another cell or tissue sample. The plurality of sets of nucleic acids can be labeled with different labels, e.g., different fluorescent labels which have distinct emission spectra so that they can be distinguished. The sets can then be mixed and hybridized simultaneously to one microarray.

For example, the two different cells can be a diseased cell of a patient having R.A. and a counterpart normal cell. Alternatively, the two different cells can be a diseased cell of a patient having R.A. and a diseased cell of a patient suspected of having R.A. In another embodiment, one biological sample is exposed to a drug and another biological sample of the same type is not exposed to the drug. The cDNA derived from each of the two cell types are differently labeled so that they can be distinguished. In one embodiment, for example, cDNA from a diseased cell is synthesized using a fluorescein-labeled dNTP, and cDNA from a second cell, i.e., the normal cell, is synthesized using a rhodamine-labeled dNTP. When the two cDNAs are mixed and hybridized to the microarray, the relative intensity of signal from each cDNA set is determined for each site on the array, and any relative difference in abundance of a particular mRNA detected.

In the example described above, the cDNA from the diseased cell will fluoresce green when the fluorophore is stimulated and the cDNA from the cell of a subject suspected of having R.A. will fluoresce red. As a result, if the two cells are essentially the same, the particular mRNA will be equally prevalent in both cells and, upon reverse transcription, red-labeled and green-labeled cDNA will be equally prevalent. When hybridized to the microarray, the binding site(s) for that species of RNA will emit wavelengths characteristic of both fluorophores (and appear brown in combination). In contrast, if the two cells are different, the ratio of green to red fluorescence will be different.

The use of a two-color fluorescence labeling and detection scheme to define alterations in gene expression has been described, e.g., in Shena et al., 1995, Quantitative monitoring of gene expression patterns with a complementary DNA microarray, Science 270:467-470. An advantage of using cDNA labeled with two different fluorophores is that a direct and internally controlled comparison of the mRNA levels corresponding to each arrayed gene in two cell states can be made, and variations due to minor differences in experimental conditions (e.g, hybridization conditions) will not affect subsequent analyses.

Examples of distinguishable labels for use when hybridizing a plurality of target nucleic acids to one array are well known in the art and include: two or more different emission wavelength fluorescent dyes, like Cy3 and Cy5, combination of fluorescent proteins and dyes, like phicoerythrin and Cy5, two or more isotopes with different energy of emission, like ³²P and ³³P, gold or silver particles with different scattering spectra, labels which generate signals under different treatment conditions, like temperature, pH, treatment by additional chemical agents, etc., or generate signals at different time points after treatment. Using one or more enzymes for signal generation allows for the use of an even greater variety of distinguishable labels, based on different substrate specificity of enzymes (alkaline phosphatase/peroxidase).

Further, it is preferable in order to reduce experimental error to reverse the fluorescent labels in two-color differential hybridization experiments to reduce biases peculiar to individual genes or array spot locations. In other words, it is preferable to first measure gene expression with one labeling (e.g., labeling nucleic acid froma first cell with a first fluorochrome and nucleic acid from a second cell with a second fluorochrome) of the mRNA from the two cells being measured, and then to measure gene expression from the two cells with reversed labeling (e.g., labeling nucleic acid from the first cell with the second fluorochrome and nucleic acid from the second cell with the first fluorochrome). Multiple measurements over exposure levels and perturbation control parameter levels provide additional experimental error control.

The quality of labeled nucleic acids can be evaluated prior to hybridization to an array. For example, a sample of the labeled nucleic acids can be hybridized to probes derived from the 5′, middle and 3′ portions of genes known to be or suspected to be present in the nucleic acid sample. This will be indicative as to whether the labeled nucleic acids are full length nucleic acids or whether they are degraded. In one embodiment, the GeneChip® Test3 Array from Affymetrix (Santa Clara, Calif.) can be used for that purpose. This array contains probes representing a subset of characterized genes from several organisms including mammals. Thus, the quality of a labeled nucleic acid sample can be determined by hybridization of a fraction of the sample to an array, such as the GeneChip® Test3 Array from Affymetrix (Santa Clara, Calif.).

(iii) Exemplary Arrays

Preferred arrays, e.g., microarrays, for use according to the invention include one or more probes of genes which are up- or down-regulated in R.A., such as one or more genes listed in any of Tables 1-5 or one or more genes characteristic of R.A. In a preferred embodiment, the array comprises probes corresponding to one or more of genes selected from the group consisting of genes which are up-regulated in R.A., e.g., genes selected from the group consisting of SOCS3 (CISH3); RAGE (AGER); LST-1 (LY117); SAA 1-3; HMG-1; S100 A8, A9, and A12; SLPI; GILZ; PTPN-18; GADD-45A and B; Legumain (PRSC1); FST1; Lcn2; GPI; SpiL; and TSG-6 and genes which are down-regulated, e.g., CAMK2B, PLA2G2A, GBAS and SOX15. The array may comprise probes corresponding to at least 10, preferably at least 20, at least 50, at least 100 or at least 1000 genes. The array may comprise probes corresponding to about 10%, 20%, 50%, 70%, 90% or 95% of the genes listed in any of Tables 1-5. The array may comprise probes corresponding to about 10%, 20%, 50%, 70%, 90% or 95% of the genes listed in any of Tables 1-5 whose expression is at least 2 fold, preferably at least 3 fold, more preferably at least 4 fold, 5 fold, 7 fold and most preferably at least about 10 fold higher in cells characteristic of R.A. relative to normal counterpart cells. One array that can be used is the array used and described in the Examples.

There can be one or more than one probe corresponding to each gene on a microarray. For example, a microarray may contain from 2 to 20 probes corresponding to one gene and preferably about 5 to 10. The probes may correspond to the full length RNA sequence or complement thereof of genes characteristic of R.A., or they may correspond to a portion thereof, which portion is of sufficient length for permitting specific hybridization. Such probes may comprise from about 50 nucleotides to about 100, 200, 500, or 1000 nucleotides or more than 1000 nucleotides. As further described herein, microarrays may contain oligonucleotide probes, consisting of about 10 to 50 nucleotides, preferably about 15 to 30 nucleotides and even more preferably 20-25 nucleotides. The probes are preferably single stranded. The probe will have sufficient complementarity to its target to provide for the desired level of sequence specific hybridization (see below).

Typically, the arrays used in the present invention will have a site density of greater than 2 100 different probes per cm . Preferably, the arrays will have a site density of greater than 500/cm², more preferably greater than about 1000/cm², and most preferably, greater than about 10,000/cm². Preferably, the arrays will have more than 100 different probes on a single substrate, more preferably greater than about 1000 different probes still more preferably, greater than about 10,000 different probes and most preferably, greater than 100,000 different probes on a single substrate.

Microarrays can be prepared by methods known in the art, as described below, or they can be custom made by companies, e.g., Affymetrix (Santa Clara, Calif.).

Generally, two types of microarrays can be used. These two types are referred to as “synthesis” and “delivery.” In the synthesis type, a microarray is prepared in a step-wise fashion by the in situ synthesis of nucleic acids from nucleotides. With each round of synthesis, nucleotides are added to growing chains until the desired length is achieved. In the delivery type of microarray, preprepared nucleic acids are deposited onto known locations using a variety of delivery technologies. Numerous articles describe the different microarray technologies, e.g., Shena et al. (1998) Tibtech 16: 301; Duggan et al. (1999) Nat. Genet. 21:10; Bowtell et al. (1999) Nat. Genet. 21: 25.

One novel synthesis technology is that developed by Affymetrix (Santa Clara, Calif.), which combines photolithography technology with DNA synthetic chemistry to enable high density oligonucleotide microarray manufacture. Such chips contain up to 400,000 groups of oligonucleotides in an area of about 1.6 cm². Oligonucleotides are anchored at the 3′ end thereby maximizing the availability of single-stranded nucleic acid for hybridization. Generally such chips, referred to as “GeneChips®” contain several oligonucleotides of a particular gene, e.g., between 15-20, such as 16 oligonucleotides. Since Affymetrix (Santa Clara, Calif.) sells custom made microarrays, microarrays containing genes which are up- or down-regulated in R.A. can be ordered for purchase from Affymetrix (Santa Clara, Calif.).

Microarrays can also be prepared by mechanical microspotting, e.g., those commercialized at Synteni (Fremont, Calif.). According to these methods, small quantities of nucleic acids are printed onto solid surfaces. Microspotted arrays prepared at Synteni contain as many as 10,000 groups of cDNA in an area of about 3.6 cm².

A third group of microarray technologies consist in the “drop-on-demand” delivery approaches, the most advanced of which are the ink-jetting technologies, which utilize piezoelectric and other forms of propulsion to transfer nucleic acids from miniature nozzles to solid surfaces. Inkjet technologies is developed at several centers including Incyte Pharmaceuticals (Palo Alto, Calif.) and Protogene (Palo Alto, Calif.). This technology results in a density of 10,000 spots per cm². See also, Hughes et al. (2001) Nat. Biotechn. 19:342.

Arrays preferably include control and reference nucleic acids. Control nucleic acids are nucleic acids which serve to indicate that the hybridization was effective. For example, all Affymetrix (Santa Clara, Calif.) expression arrays contain sets of probes for several prokaryotic genes, e.g., bioB, bioC and bioD from biotin synthesis of E. coli and cre from P1 bacteriophage. Hybridization to these arrays is conducted in the presence of a mixture of these genes or portions thereof, such as the mix provided by Affymetrix (Santa Clara, Calif.) to that effect (Part Number 900299), to thereby confirm that the hybridization was effective. Control nucleic acids included with the target nucleic acids can also be mRNA synthesized from cDNA clones by in vitro transcription. Other control genes that may be included in arrays are polyA controls, such as dap, lys, phe, thr, and trp (which are included on Affymetrix GeneChips®).

Reference nucleic acids allow the normalization of results from one experiment to another, and to compare multiple experiments on a quantitative level. Exemplary reference nucleic acids include housekeeping genes of known expression levels, e.g., GAPDH, hexokinase and actin.

Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases.

Arrays may also contain probes that hybridize to more than one allele of a gene. For example the array can contain one probe that recognizes allele 1 and another probe that recognizes allele 2 of a particular gene.

Microarrays can be prepared as follows. In one embodiment, an array of oligonucleotides is synthesized on a solid support. Exemplary solid supports include glass, plastics, polymers, metals, metalloids, ceramics, organics, etc. Using chip masking technologies and photoprotective chemistry it is possible to generate ordered arrays of nucleic acid probes. These arrays, which are known, e.g., as “DNA chips,” or as very large scale immobilized polymer arrays (“VLSIPS™” arrays) can include millions of defined probe regions on a substrate having an area of about 1 cm² to several cm², thereby incorporating sets of from a few to millions of probes (see, e.g., U.S. Pat. No. 5,631,734).

The construction of solid phase nucleic acid arrays to detect target nucleic acids is well described in the literature. See, Fodor et al. (1991) Science, 251: 767-777; Sheldon et al. (1993) Clinical Chemistry 39(4): 718-719; Kozal et al. (1996) Nature Medicine 2(7): 753-759 and Hubbell U.S. Pat. No. 5,571,639; Pinkel et al. PCT/US95/16155 (WO 96/17958); U.S. Pat. Nos. 5,677,195; 5,624,711; 5,599,695; 5,451,683; 5,424,186; 5,412,087; 5,384,261; 5,252,743 and 5,143,854; PCT Patent Publication Nos. 92/10092 and 93/09668; and PCT WO 97/10365. In brief, a combinatorial strategy allows for the synthesis of arrays containing a large number of probes using a minimal number of synthetic steps. For instance, it is possible to synthesize and attach all possible DNA 8 mer oligonucleotides (48, or 65,536 possible combinations) using only 32 chemical synthetic steps. In general, VLSIPS™ procedures provide a method of producing 4n different oligonucleotide probes on an array using only 4n synthetic steps (see, e.g., U.S. Pat. No. 5,631,734 5,143,854 and PCT Patent Publication Nos. WO 90/15070; WO 95/11995 and WO 92/10092).

Light-directed combinatorial synthesis of oligonucleotide arrays on a glass surface can be performed with automated phosphoramidite chemistry and chip masking techniques similar to photoresist technologies in the computer chip industry. Typically, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′-photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface.

Algorithms for design of masks to reduce the number of synthesis cycles are described by Hubbel et al., U.S. Pat. No. 5,571,639 and U.S. Pat. No. 5,593,839. A computer system may be used to select nucleic acid probes on the substrate and design the layout of the array as described in U.S. Pat. No. 5,571,639.

Another method for synthesizing high density arrays is described in U.S. Pat. No. 6,083,697. This method utilizes a novel chemical amplification process using a catalyst system which is initiated by radiation to assist in the synthesis the polymer sequences. Such methods include the use of photosensitive compounds which act as catalysts to chemically alter the synthesis intermediates in a manner to promote formation of polymer sequences. Such photosensitive compounds include what are generally referred to as radiation-activated catalysts (RACs), and more specifically photo activated catalysts (PACs). The RACs can by themselves chemically alter the synthesis intermediate or they can activate an autocatalytic compound which chemically alters the synthesis intermediate in a manner to allow the synthesis intermediate to chemically combine with a later added synthesis intermediate or other compound.

Arrays can also be synthesized in a combinatorial fashion by delivering monomers to cells of a support by mechanically constrained flowpaths. See Winkler et al., EP 624,059. Arrays can also be synthesized by spotting monomers reagents on to a support using an ink jet printer. See id. and Pease et al., EP 728,520. cDNA probes can be prepared according to methods known in the art and further described herein, e.g., reverse-transcription PCR (RT-PCR) of RNA using sequence specific primers. Oligonucleotide probes can be synthesized chemically. Sequences of the genes or cDNA from which probes are made can be obtained, e.g., from GenBank, other public databases or publications.

Nucleic acid probes can be natural nucleic acids, chemically modified nucleic acids, e.g., composed of nucleotide analogs, as long as they have activated hydroxyl groups compatible with the linking chemistry. The protective groups can, themselves, be photolabile. Alternatively, the protective groups can be labile under certain chemical conditions, e.g., acid. In this example, the surface of the solid support can contain a composition that generates acids upon exposure to light. Thus, exposure of a region of the substrate to light generates acids in that region that remove the protective groups in the exposed region. Also, the synthesis method can use 3′-protected 5′-0-phosphoramidite-activated deoxynucleoside. In this case, the oligonucleotide is synthesized in the 5′ to 3′ direction, which results in a free 5′ end.

Oligonucleotides of an array can be synthesized using a 96 well automated multiplex oligonucleotide synthesizer (A.M.O.S.) that is capable of making thousands of oligonucleotides (Lashkari et al. (1995) PNAS 93: 7912) can be used.

It will be appreciated that oligonucleotide design is influenced by the intended application. For example, it may be desirable to have similar melting temperatures for all of the probes. Accordingly, the length of the probes are adjusted so that the melting temperatures for all of the probes on the array are closely similar (it will be appreciated that different lengths for different probes may be needed to achieve a particular T[m] where different probes have different GC contents). Although melting temperature is a primary consideration in probe design, other factors are optionally used to further adjust probe construction, such as selecting against primer self-complementarity and the like.

Arrays, e.g., microarrrays, may conveniently be stored following fabrication or purchase for use at a later time. Under appropriate conditions, the subject arrays are capable of being stored for at least about 6 months and may be stored for up to one year or longer. Arrays are generally stored at temperatures between about −20° C. to room temperature, where the arrays are preferably sealed in a plastic container, e.g. bag, and shielded from light.

(iv) Hybridization of the Target Nucleic Acids to the Microarray

The next step is to contact the target nucleic acids with the array under conditions sufficient for binding between the target nucleic acids and the probes of the array. In a preferred embodiment, the target nucleic acids will be contacted with the array under conditions sufficient for hybridization to occur between the target nucleic acids and probes on the microarray, where the hybridization conditions will be selected in order to provide for the desired level of hybridization specificity.

Contact of the array and target nucleic acids involves contacting the array with an aqueous medium comprising the target nucleic acids. Contact may be achieved in a variety of different ways depending on specific configuration of the array. For example, where the array simply comprises the pattern of size separated probes on the surface of a “plate-like” rigid substrate, contact may be accomplished by simply placing the array in a container comprising the target nucleic acid solution, such as a polyethylene bag, and the like. In other embodiments where the array is entrapped in a separation media bounded by two rigid plates, the opportunity exists to deliver the target nucleic acids via electrophoretic means. Alternatively, where the array is incorporated into a biochip device having fluid entry and exit ports, the target nucleic acid solution can be introduced into the chamber in which the pattern of target molecules is presented through the entry port, where fluid introduction could be performed manually or with an automated device. In multiwell embodiments, the target nucleic acid solution will be introduced in the reaction chamber comprising the array, either manually, e.g. with a pipette, or with an automated fluid handling device.

Contact of the target nucleic acid solution and the probes will be maintained for a sufficient period of time for binding between the target and the probe to occur. Although dependent on the nature of the probe and target, contact will generally be maintained for a period of time ranging from about 10 min to 24 hrs, usually from about 30 min to 12 hrs and more usually from about 1 hr to 6 hrs.

When using commercially available microarrays, adequate hybridization conditions are provided by the manufacturer. When using non-commercial microarrays, adequate hybridization conditions can be determined based on the following hybridization guidelines, as well as on the hybridization conditions described in the numerous published articles on the use of microarrays.

Nucleic acid hybridization and wash conditions are optimally chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls.

Hybridization is carried out in conditions permitting essentially specific hybridization. The length of the probe and GC content will determine the Tm of the hybrid, and thus the hybridization conditions necessary for obtaining specific hybridization of the probe to the template nucleic acid. These factors are well known to a person of skill in the art, and can also be tested in assays. An extensive guide to the hybridization of nucleic acids is found in Tijssen (1993), “Laboratory Techniques in biochemistry and molecular biology-hybridization with nucleic acid probes.” Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Highly stringent conditions are selected to be equal to the Tm point for a particular probe. Sometimes the term “Td” is used to define the temperature at which at least half of the probe dissociates from a perfectly matched target nucleic acid. In any case, a variety of estimation techniques for estimating the Tm or Td are available, and generally described in Tijssen, supra. Typically, G-C base pairs in a duplex are estimated to contribute about 3° C. to the Tm, while A-T base pairs are estimated to contribute about 2° C., up to a theoretical maximum of about 80-100° C. However, more sophisticated models of Tm and Td are available and appropriate in which G-C stacking interactions, solvent effects, the desired assay temperature and the like are taken into account. For example, probes can be designed to have a dissociation temperature (Td) of approximately 60° C., using the formula: Td=(((((3×#GC)+(2×#AT))×37)-562)/#bp)-5; where #GC, #AT, and #bp are the number of guanine-cytosine base pairs, the number of adenine-thymine base pairs, and the number of total base pairs, respectively, involved in the annealing of the probe to the template DNA.

The stability difference between a perfectly matched duplex and a mismatched duplex, particularly if the mismatch is only a single base, can be quite small, corresponding to a difference in Tm between the two of as little as 0.5 degrees. See Tibanyenda, N. et al., Eur. J. Biochem. 139:19 (1984) and Ebel, S. et al., Biochem. 31:12083 (1992). More importantly, it is understood that as the length of the homology region increases, the effect of a single base -mismatch on overall duplex stability decreases.

Theory and practice of nucleic acid hybridization is described, e.g., in S. Agrawal (ed.) Methods in Molecular Biology, volume 20; and Tijssen (1993) Laboratory Techniques in biochemistry and molecular biology-hybridization with nucleic acid probes, e.g., part I chapter 2 “Overview of principles of hybridization and the strategy of nucleic acid probe assays”, Elsevier, New York provide a basic guide to nucleic acid hybridization.

Certain microarrays are of “active” nature, i.e., they provide independent electronic control over all aspects of the hybridization reaction (or any other affinity reaction) occurring at each specific microlocation. These devices provide a new mechanism for affecting hybridization reactions which is called electronic stringency control (ESC). Such active devices can electronically produce “different stringency conditions” at each microlocation. Thus, all hybridizations can be carried out optimally in the same bulk solution. These arrays are described in U.S. Pat. No. 6,051,380 by Sosnowski et al.

In a preferred embodiment, background signal is reduced by the use of a detergent (e.g, C-TAB) or a blocking reagent (e.g., sperm DNA, cot-i DNA, etc.) during the hybridization to reduce non-specific binding. In a particularly preferred (embodiment, the hybridization is performed in the presence of about 0.5 mg/ml DNA (e.g., herring sperm DNA). The use of blocking agents in hybridization is well known to those of skill in the art (see, e.g., Chapter 8 in Laboratory Techniques in Biochemistry and Molecular Biology, Vol. 24: Hybridization With Nucleic Acid Probes, P. Tijssen, ed. Elsevier, N.Y., (1993)).

The method may or may not further comprise a non-bound label removal step prior to the detection step, depending on the particular label employed on the target nucleic acid. For example, in certain assay formats (e.g., “homogenous assay formats”) a detectable signal is only generated upon specific binding of target to probe. As such, in these assay formats, the hybridization pattern may be detected without a non-bound label removal step. In other embodiments, the label employed will generate a signal whether or not the target is specifically bound to its probe. In such embodiments, the non-bound labeled target is removed from the support surface. One means of removing the non-bound labeled target is to perform the well known technique of washing, where a variety of wash solutions and protocols for their use in removing non-bound label are known to those of skill in the art and may be used. Alternatively, non-bound labeled target can be removed by electrophoretic means.

Where all of the target sequences are detected using the same label, different arrays will be employed for each physiological source (where different could include using the same array at different times). The above methods can be varied to provide for multiplex analysis, by employing different and distinguishable labels for the different target populations (representing each of the different physiological sources being assayed). According to this multiplex method, the same array is used at the same time for each of the different target populations.

In another embodiment, hybridization is monitored in real time using a charge-coupled device (CCD) imaging camera (Guschin et al. (1997) Anal. Biochem. 250:203). Synthesis of arrays on optical fibre bundles allows easy and sensitive reading (Healy et al. (1997) Anal. Biochem. 251:270). In another embodiment, real time hybridization detection is carried out on microarrays without washing using evanescent wave effect that excites only fluorophores that are bound to the surface (see, e.g., Stimpson et al. (1995) PNAS 92:6379).

(v) Detection of Hybridization and Analysis of Results

The above steps result in the production of hybridization patterns of target nucleic acid on the array surface. These patterns may be visualized or detected in a variety of ways, with the particular manner of detection being chosen based on the particular label of the target nucleic acid. Representative detection means include scintillation counting, autoradiography, fluorescence measurement, colorimetric measurement, light emission measurement, light scattering, and the like.

One method of detection includes an array scanner that is commercially available from Affymetrix (Santa Clara, Calif.), e.g., the ₄₁₇™ Arrayer, the 418™ Array Scanner, or the Agilent GeneArray™ Scanner. This scanner is controlled from the system computer with a Windows^{R} interface and easy-to-use software tools. The output is a 16-bit.tif file that can be directly imported into or directly read by a variety of software applications. Preferred scanning devices are described in, e.g., U.S. Pat. Nos. 5,143,854 and 5,424,186.

When fluorescently labeled probes are used, the fluorescence emissions at each site of a transcript array can be detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al., 1996, A DNA microarray system for analyzing complex DNA samples using two-color fluorescent probe hybridization, Genome Research 6:639-645). In a preferred embodiment, the arrays are scanned with a laser fluorescent scanner with a computer controlled X-Y stage and a microscope objective. Sequential excitation of the two fluorophores can be achieved with a multi-line, mixed gas laser and the emitted light is split by wavelength and detected with two photomultiplier tubes. In one embodiment in which fluorescent target nucleic acids are used, the arrays may be scanned using lasers to excite fluorescently labeled targets that have hybridized to regions of probe arrays, which can then be imaged using charged coupled devices (“CCDs”) for a wide field scanning of the array. Fluorescence laser scanning devices are described, e.g., in Schena et al., 1996, Genome Res. 6:639-645. Alternatively, the fiber-optic bundle described by Ferguson et al., 1996, Nature Biotech. 14:1681-1684, may be used to monitor mRNA abundance levels.

Following the data gathering operation, the data will typically be reported to a data analysis operation. To facilitate the sample analysis operation, the data obtained by the reader from the device will typically be analyzed using a digital computer. Typically, the computer will be appropriately programmed for receipt and storage of the data from the device, as well as for analysis and reporting of the data gathered, e.g., subtrackion of the background, deconvolution multi-color images, flagging or removing artifacts, verifying that controls have performed properly, normalizing the signals, interpreting fluorescence data to determine the amount of hybridized target, normalization of background and single base mismatch hybridizations, and the like. In a preferred embodiment, a system comprises a search function that allows one to search for specific patterns, e.g., patterns relating to differential gene expression, e.g., between the expression profile of a cell of R.A. and the expression profile of a counterpart normal cell in a subject. A system preferably allows one to search for patterns of gene expression between more than two samples.

A desirable system for analyzing data is a general and flexible system for the visualization, manipulation, and analysis of gene expression data. Such a system preferably includes a graphical user interface for browsing and navigating through the expression data, allowing a user to selectively view and highlight the genes of interest. The system also preferably includes sort and search functions and is preferably available for general users with PC, Mac or Unix workstations. Also preferably included in the system are clustering algorithms that are qualitatively more efficient than existing ones. The accuracy of such algorithms is preferably hierarchically adjustable so that the level of detail of clustering can be systematically refined as desired.

Various algorithms are available for analyzing the gene expression profile data, e.g., the type of comparisons to perform. In certain embodiments, it is desirable to group genes that are co-regulated. This allows the comparison of large numbers of profiles. A preferred embodiment for identifying such groups of genes involves clustering algorithms (for reviews of clustering algorithms, see, e.g., Fukunaga, 1990, Statistical Pattern Recognition, 2nd Ed., Academic Press, San Diego; Everitt, 1974, Cluster Analysis, London: Heinemann Educ. Books; Hartigan, 1975, Clustering Algorithms, New York: Wiley; Sneath and Sokal, 1973, Numerical Taxonomy, Freeman; Anderberg, 1973, Cluster Analysis for Applications, Academic Press: New York).

Clustering analysis is useful in helping to reduce complex patterns of thousands of time curves into a smaller set of representative clusters. Some systems allow the clustering and viewing of genes based on sequences. Other systems allow clustering based on other characteristics of the genes, e.g., their level of expression (see, e.g. U.S. Pat. No. 6,203,987). Other systems permit clustering of time curves (see, e.g. U.S. Pat. No. 6,263,287). Cluster analysis can be performed using the hclust routine (see, e.g., “hclust” routine from the software package S-Plus, MathSoft, Inc., Cambridge, Mass.).

In some specific embodiments, genes are grouped according to the degree of co-variation of their transcription, presumably co-regulation, as described in U.S. Pat. No. 6,203,987. Groups of genes that have co-varying transcripts are termed “genesets.” Cluster analysis or other statistical classification methods can be used to analyze the co-variation of transcription of genes in response to a variety of perturbations, e.g. caused by a disease or a drug. In one specific embodiment, clustering algorithms are applied to expression profiles to construct a “similarity tree” or “clustering tree” which relates genes by the amount of co-regulation exhibited. Genesets are defined on the branches of a clustering tree by cutting across the clustering tree at different levels in the branching hierarchy.

In some embodiments, a gene expression profile is converted to a projected gene expression profile. The projected gene expression profile is a collection of geneset expression values. The conversion is achieved, in some embodiments, by averaging the level of expression of the genes within each geneset. In some other embodiments, other linear projection processes may be used. The projection operation expresses the profile on a smaller and biologically more meaningful set of coordinates, reducing the effects of measurement errors by averaging them over each cellular constituent sets and aiding biological interpretation of the profile.

Values that can be compared include gross expression levels; averages of expression levels, e.g., from different experiments, different samples from the same subject or samples from different subjects; and ratios of expression levels, e.g., between R.A. subjects and normal controls, between different R.A. subjects and isolated cell populations.

2.2. Other Methods for Determining Gene Expression Levels

In certain embodiments, it is sufficient to determine the expression of one or only a few genes, as opposed to hundreds or thousands of genes. Although microarrays can be used in these embodiments, various other methods of detection of gene expression are available. This section describes a few exemplary methods for detecting and quantifying mRNA or polypeptide encoded thereby. Where the first step of the methods includes isolation of mRNA from cells, this step can be conducted as described above. Labeling of one or more nucleic acids can be performed as described above.

In one embodiment, mRNA obtained form a sample is reverse transcribed into a first cDNA strand and subjected to PCR, e.g., RT-PCR. House keeping genes, or other genes whose expression does not vary can be used as internal controls and controls across experiments. Following the PCR reaction, the amplified products can be separated by electrophoresis and detected. By using quantitative PCR, the level of amplified product will correlate with the level of RNA that was present in the sample. The amplified samples can also be separated on a agarose or polyacrylamide gel, transferred onto a filter, and the filter hybridized with a probe specific for the gene of interest. Numerous samples can be analyzed simultaneously by conducting parallel PCR amplification, e.g., by multiplex PCR.

A quantitative PCR technique that can be used is based on the use of TaqMan probes. Specific sequence detection occurs by amplification of target sequences in the PE Applied Biosystems 7700 Sequence Detection System in the presence of an oligonucleotide probe labeled at the 5′ and 3′ ends with a reporter and quencher fluorescent dye, respectively (FQ probe), which anneals between the two PCR primers. Only specific product will be detected when the probe is bound between the primers. As PCR amplification proceeds, the 5′-nuclease activity of Taq polymerase initially cleaves the reporter dye from the probe. The signal generated when the reporter dye is physically separated from the quencher dye is detected by measuring the signal with an attached CCD camera. Each signal generated equals one probe cleaved which corresponds to amplification of one target strand. PCR reactions may be set up using the PE Applied Biosystem TaqMan PCR Core Reagent Kit according to the instructions supplied. This technique is further described, e.g., in U.S. Pat. No. 6,326,462.

In another embodiment, mRNA levels is determined by dotblot analysis and related methods (see, e.g., G. A. Beltz et al., in Methods in Enzymology, Vol. 100, Part B, R. Wu, L. Grossmam, K. Moldave, Eds., Academic Press, New York, Chapter 19, pp. 266-308, 1985). In one embodiment, a specified amount of RNA extracted from cells is blotted (i.e., non-covalently bound) onto a filter, and the filter is hybridized with a probe of the gene of interest. Numerous RNA samples can be analyzed simultaneously, since a blot can comprise multiple spots of RNA. Hybridization is detected using a method that depends on the type of label of the probe. In another dotblot method, one or more probes of one or more genes which are up- or down-regulated in R.A. are attached to a membrane, and the membrane is incubated with labeled nucleic acids obtained from and optionally derived from RNA of a cell or tissue of a subject. Such a dotblot is essentially an array comprising fewer probes than a microarray.

“Dot blot” hybridization gained wide-spread use, and many versions were developed (see, e.g., M. L. M. Anderson and B. D. Young, in Nucleic Acid Hybridization-A Practical Approach, B. D. Hames and S. J. Higgins, Eds., IRL Press, Washington D.C., Chapter 4, pp. 73-111, 1985).

Another format, the so-called “sandwich” hybridization, involves covalently attaching oligonucleotide probes to a solid support and using them to capture and detect multiple nucleic acid targets (see, e.g., M. Ranki et al., Gene, 21, pp. 77-85, 1983; A. M. Palva, T. M. Ranki, and H. E. Soderlund, in UK Patent Application GB 2156074A, Oct. 2, 1985; T. M. Ranki and H. E. Soderlund in U.S. Pat. No. 4,563,419, Jan. 7, 1986; A. D. B. Malcolm and J. A. Langdale, in PCT WO 86/03782, Jul. 3, 1986; Y. Stabinsky, in U.S. Pat. No. 4,751,177, Jan. 14, 1988; T. H. Adams et al., in PCT WO 90/01564, Feb. 22, 1990; R. B. Wallace et al. 6 Nucleic Acid Res. 11, p. 3543, 1979; and B. J. Connor et al., 80 Proc. Natl. Acad. Sci. USA pp. 278-282, 1983). Multiplex versions of these formats are called “reverse dot blots.” mRNA levels can also be determined by Northern blots. Specific amounts of RNA are separated by gel electrophoresis and transferred onto a filter which is then hybridized with a probe corresponding to the gene of interest. This method, although more burdensome when numerous samples and genes are to be analyzed provides the advantage of being very accurate.

A preferred method for high throughput analysis of gene expression is the serial analysis of gene expression (SAGE) technique, first described in Velculescu et al. (1995) Science 270, 484-487. Among the advantages of SAGE is that it has the potential to provide detection of all genes expressed in a given cell type, provides quantitative information about the relative expression of such genes, permits ready comparison of gene expression of genes in two cells, and yields sequence information that can be used to identify the detected genes. Thus far, SAGE methodology has proved itself to reliably detect expression of regulated and nonregulated genes in a variety of cell types (Velculescu et al. (1997) Cell 88, 243-251; Zhang et al. (1997) Science 276, 1268-1272 and Velculescu et al. (1999) Nat. Genet. 23, 387-388).

Techniques for producing and probing nucleic acids are further described, for example, in Sambrook et al., “Molecular Cloning: A Laboratory Manual” (New York, Cold Spring Harbor Laboratory, 1989).

Alternatively, the level of expression of one or more genes which are up- or down-regulated in R.A. is determined by in situ hybridization. In one embodiment, a tissue sample is obtained from a subject, the tissue sample is sliced, and in situ hybridization is performed according to methods known in the art, to determine the level of expression of the genes of interest.

In other methods, the level of expression of a gene is detected by measuring the level of protein encoded by the gene. This can be done, e.g., by immunoprecipitation, ELISA, or immunohistochemistry using an agent, e.g., an antibody, that specifically detects the protein encoded by the gene. Other techniques include Western blot analysis. Immunoassays are commonly used to quantitate the levels of proteins in cell samples, and many other immunoassay techniques are known in the art. The invention is not limited to a particular assay procedure, and therefore is intended to include both homogeneous and heterogeneous procedures. Exemplary immunoassays which can be conducted according to the invention include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art.

In the case of polypeptides which are secreted from cells, the level of expression of these polypeptides can be measured in biological fluids.

2.3. Data Analysis Methods

Comparison of the expression levels of one or more genes which are up- or down-regulated in R.A. with reference expression levels, e.g., expression levels in cells characteristic of R.A. or in normal counterpart cells, is preferably conducted using computer systems. In one embodiment, one or more expression levels are obtained in two cells and these two sets of expression levels are introduced into a computer system for comparison. In a preferred embodiment, one set of one or more expression levels is entered into a computer system for comparison with values that are already present in the computer system, or in computer-readable form that is then entered into the computer system.

In one embodiment, the invention provides a computer readable form of the gene expression profile data of the invention, or of values corresponding to the level of expression of at least one gene which is up- or down-regulated in R.A. The values can be mRNA expression levels obtained from experiments, e.g., microarray analysis. The values can also be mRNA levels normalized relative to a reference gene whose expression is constant in numerous cells under numerous conditions, e.g., GAPDH. In other embodiments, the values in the computer are ratios of, or differences between, normalized or non-normalized mRNA levels in different samples.

The computer readable medium may comprise values of at least 2, at least 3, at least 5, 10, 20, 50, 100, 200, 500 or more genes, e.g., genes listed in Tables 1-5. In a preferred embodiment, the computer readable medium comprises at least one expression profile.

Gene expression data can be in the form of a table, such as an Excel table. The data can be alone, or it can be part of a larger database, e.g., comprising other expression profiles, e.g., publicly available database. The computer readable form can be in a computer. In another embodiment, the invention provides a computer displaying the gene expression profile data.

Although the invention provides methods in which the level of expression of a single gene can be compared in two or more cells or tissue samples, in a preferred embodiment, the level of expression of a plurality of genes is compared. For example, the level of expression of at least 2, at least 3, at least 5, 10, 20, 50, 100, 200, 500 or more genes, e.g., genes listed in Tables 1-5 can be compared. In a preferred embodiment, expression profiles are compared.

In one embodiment, the invention provides a method for determining the similarity between the level of expression of one or more genes which are up- or down-regulated in R.A. in a first cell, e.g., a cell of a subject, and that in a second cell. The method preferably comprises obtaining the level of expression of one or more genes which are up- or down-regulated in R.A. in a first cell and entering these values into a computer comprising (i) a database including records comprising values corresponding to levels of expression of one or more genes which are up- or down-regulated in R.A. in a second cell, and (ii) processor instructions, e.g., a user interface, capable of receiving a selection of one or more values for comparison purposes with data that is stored in the computer. The computer may further comprise a means for converting the comparison data into a diagram or chart or other type of output.

In another embodiment, values representing expression levels of one or more genes which are up- or down-regulated in R.A. are entered into a computer system which comprises one or more databases with reference expression levels obtained from more than one cell. For example, the computer may comprise expression data of diseased and normal cells. Instructions are provided to the computer, and the computer is capable of comparing the data entered with the data in the computer to determine whether the data entered is more similar to that of a normal cell or to that of a diseased cell.

In another embodiment, the computer comprises values of expression levels in cells of subjects at different stages of R.A., and the computer is capable of comparing expression data entered into the computer with the data stored, and produce results indicating to which of the expression data in the computer, the one entered is most similar, such as to determine the stage of R.A. in the subject.

In yet another embodiment, the reference expression data in the computer are expression data from cells of R.A. of one or more subjects, which cells are treated in vivo or in vitro with a drug used for therapy of R.A. Upon entering of expression data of a cell of a subject treated in vitro or in vivo with the drug, the computer is instructed to compare the data entered with the data in the computer, and to provide results indicating whether the expression data input into the computer are more similar to those of a cell of a subject that is responsive to the drug or more similar to those of a cell of a subject that is not responsive to the drug. Thus, the results indicate whether the subject is likely to respond to the treatment with the drug or unlikely to respond to it.

The reference expression data may also be from cells from subjects responding or not responding to several different treatments, and the computer system indicates a preferred treatment for the subject. Accordingly, the invention provides a method for selecting a therapy for a patient having R.A., the method comprising: (i) providing the level of expression of one or more genes which are up- or down-regulated in R.A. in a diseased cell of the patient; (ii) providing a plurality of reference expression levels, each associated with a therapy, wherein the subject expression levels and each reference expression level has a plurality of values, each value representing the level of expression of a gene that is up- or down-regulated in R.A.; and (iii) selecting the reference expression levels most similar to the subject expression levels, to thereby select a therapy for said patient. In a preferred embodiment step (iii) is performed by a computer. The most similar reference profile may be selected by weighing a comparison value of the plurality using a weight value associated with the corresponding expression data.

In one embodiment, the invention provides a system that comprises a means for receiving gene expression data for one or a plurality of genes; a means for comparing the gene expression data from each of said one or plurality of genes to a common reference frame; and a means for presenting the results of the comparison. This system may further comprise a means for clustering the data.

In another embodiment, the invention provides a computer program for analyzing gene expression data comprising (i) a computer code that receives as input gene expression data for a plurality of genes and (ii) a computer code that compares said gene expression data from each of said plurality of genes to a common reference frame.

The invention also provides a machine-readable or computer-readable medium including program instructions for performing the following steps: (i) comparing a plurality of values corresponding to expression levels of one or more genes which are up- or down-regulated in R.A. in a query cell with a database including records comprising reference expression of one or more reference cells and an annotation of the type of cell; and (ii) indicating to which cell the query cell is most similar based on similarities of expression levels.

The relative levels of expression, e.g., abundance of an mRNA, in two biological samples can be scored as a perturbation (relative abundance difference) or as not perturbed (i.e., the relative abundance is the same). For example, a perturbation can be a difference in expression levels between the two sources of RNA of at least a factor of about 25% (RNA from one source is 25% more abundant in one source than the other source), more usually about 50%, even more often by a factor of about 2 (twice as abundant), 3 (three times as abundant) or 5 (five times as abundant). Perturbations can be used by a computer for calculating and expressing comparisons.

Preferably, in addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

The computer readable medium may further comprise a pointer to a descriptor of the level of expression or expression profile, e.g., from which source it was obtained, e.g., from which patient it was obtained. A descriptor can reflect the stage of R.A., the therapy that the patient is undergoing or any other descriptions of the source of expression levels.

In operation, the means for receiving gene expression data, the means for comparing the gene expression data, the means for presenting, the means for normalizing, and the means for clustering within the context of the systems of the present invention can involve a programmed computer with the respective functionalities described herein, implemented in hardware or hardware and software; a logic circuit or other component of a programmed computer that performs the operations specifically identified herein, dictated by a computer program; or a computer memory encoded with executable instructions representing a computer program that can cause a computer to function in the particular fashion described herein.

Those skilled in the art will understand that the systems and methods of the present invention may be applied to a variety of systems, including IBM-compatible personal computers running MS-DOS or Microsoft Windows.

The computer may have internal components linked to external components. The internal components may include a processor element interconnected with a main memory. The computer system can be an Intel Pentium®-based processor of 200 MHz or greater clock rate and with 32 MB or more of main memory. The external component may comprise a mass storage, which can be one or more hard disks (which are typically packaged together with the processor and memory). Such hard disks are typically of 1 GB or greater storage capacity. Other external components include a user interface device, which can be a monitor, together with an inputing device, which can be a “mouse”, or other graphic input devices, and/or a keyboard. A printing device can also be attached to the computer.

Typically, the computer system is also linked to a network link, which can be part of an Ethernet link to other local computer systems, remote computer systems, or wide area communication networks, such as the Internet. This network link allows the computer system to share data and processing tasks with other computer systems.

Loaded into memory during operation of this system are several software components, which are both standard in the art and special to the instant invention. These software components collectively cause the computer system to function according to the methods of this invention. These software components are typically stored on a mass storage. A software component represents the operating system, which is responsible for managing the computer system and its network interconnections. This operating system can be, for example, of the Microsoft Windows' family, such as Windows 95, Windows 98, or Windows NT. A software component represents common languages and functions conveniently present on this system to assist programs implementing the methods specific to this invention. Many high or low level computer languages can be used to program the analytic methods of this invention. Instructions can be interpreted during run-time or compiled. Preferred languages include C/C++, and JAVA®. Most preferably, the methods of this invention are programmed in mathematical software packages which allow symbolic entry of equations and high-level specification of processing, including algorithms to be used, thereby freeing a user of the need to procedurally program individual equations or algorithms. Such packages include Matlab from Mathworks (Natick, Mass.), Mathematica from Wolfram Research (Champaign, Ill.), or S-Plus from Math Soft (Cambridge, Mass.). Accordingly, a software component represents the analytic methods of this invention as programmed in a procedural language or symbolic package. In a preferred embodiment, the computer system also contains a database comprising values representing levels of expression of one or more genes which are up- or down-regulated in R.A. The database may contain one or more expression profiles of genes which are up- or down-regulated in R.A. in different cells.

In an exemplary implementation, to practice the methods of the present invention, a user first loads expression data into the computer system. These data can be directly entered by the user from a monitor and keyboard, or from other computer systems linked by a network connection, or on removable storage media such as a CD-ROM or floppy disk or through the network. Next the user causes execution of expression profile analysis software which performs the steps of comparing and, e.g., clustering co-varying genes into groups of genes.

In another exemplary implementation, expression profiles are compared using a method described in U.S. Pat. No. 6,203,987. A user first loads expression profile data into the computer system. Geneset profile definitions are loaded into the memory from the storage media or from a remote computer, preferably from a dynamic geneset database system, through the network. Next the user causes execution of projection software which performs the steps of converting expression profile to projected expression profiles. The projected expression profiles are then displayed.

In yet another exemplary implementation, a user first leads a projected profile into the memory. The user then causes the loading of a reference profile into the memory. Next, the user causes the execution of comparison software which performs the steps of objectively comparing the profiles.

3. Exemplary Diagnostic and Prognostic Compositions and Devices of the Invention

Any composition and device (e.g., an array) used in the above-described methods are within the scope of the invention.

In one embodiment, the invention provides a composition comprising a plurality of detection agents for detecting expression of genes which are up- or down-regulated in R.A. In a preferred embodiment, the composition comprises at least 2, preferably at least 3, 5, 10, 20, 50, or 100 different detection agents. A detection agent can be a nucleic acid probe, e.g., DNA or RNA, or it can be a polypeptide, e.g., as antibody that binds to the polypeptide encoded by a gene characteristic of R.A. The probes can be present in equal amount or in different amounts in the solution.

A nucleic acid probe can be at least about 10 nucleotides long, preferably at least about 15, 20, 25, 30, 50, 100 nucleotides or more, and can comprise the full length gene. Preferred probes are those that hybridize specifically to genes listed in any of Tables 1-5. If the nucleic acid is short (i.e., 20 nucleotides or less), the sequence is preferably perfectly complementary to the target gene (i.e., a gene that is characteristic of R.A.), such that specific hybridization can be obtained. However, nucleic acids, even short ones that are not perfectly complementary to the target gene can also be included in a composition of the invention, e.g., for use as a negative control. Certain compositions may also comprise nucleic acids that are complementary to, and capable of detecting, an allele of a gene.

In a preferred embodiment, the invention provides nucleic acids which hybridize under high stringency conditions of 0.2 to 1× SSC at 65° C. followed by a wash at 0.2× SSC at 65° C. to genes which are up- or down-regulated in R.A. In another embodiment, the invention provides nucleic acids which hybridize under low stringency conditions of 6× SSC at room temperature followed by a wash at 2× SSC at room temperature. Other nucleic acids probes hybridize to their target in 3× SSC at 40 or 50° C., followed by a wash in 1 or 2× SSC at 20, 30, 40, 50, 60, or 65° C.

Nucleic acids which are at least about 80%, preferably at least about 90%, even more preferably at least about 95% and most preferably at least about 98% identical to genes which are up- or down-regulated in R.A. or cDNAs thereof, and complements thereof, are also within the scope of the invention.

Nucleic acid probes can be obtained by, e.g., polymerase chain reaction (PCR) amplification of gene segments from genomic DNA, cDNA (e.g., by RT-PCR), or cloned sequences. PCR primers are chosen, based on the known sequence of the genes or cDNA, that result in amplification of unique fragments. Computer programs can be used in the design of primers with the required specificity and optimal amplification properties. See, e.g., Oligo version 5.0 (National Biosciences). Factors which apply to the design and selection of primers for amplification are described, for example, by Rylchik, W. (1993) “Selection of Primers for Polymerase Chain Reaction,” in Methods in Molecular Biology, Vol. 15, White B. ed., Humana Press, Totowa, N.J. Sequences can be obtained from GenBank or other public sources.

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16: 3209), methylphosphonate oligonucleotides can be prepared by use of controlled pore glass polymer supports (Sarin et al., 1988, Proc. Nat. Acad. Sci. U.S.A. 85: 7448-7451), etc. In another embodiment, the oligonucleotide is a 2′-0-methylribonucleotide (Inoue et al., 1987, Nucl. Acids Res. 15: 6131-6148), or a chimeric RNA-DNA analog (Inoue et al., 1987, FEBS Lett. 215: 327-330).

“Rapid amplification of cDNA ends,” or RACE, is a PCR method that can be used for amplifying cDNAs from a number of different RNAs. The cDNAs may be ligated to an oligonucleotide linker and amplified by PCR using two primers. One primer may be based on sequence from the instant nucleic acids, for which full length sequence is desired, and a second primer may comprise a sequence that hybridizes to the oligonucleotide linker to amplify the cDNA. A description of this method is reported in PCT Pub. No. WO 97/19110.

In another embodiment, the invention provides a composition comprising a plurality of agents which can detect a polypeptide encoded by a gene characteristic of R.A. An agent can be, e.g., an antibody. Antibodies to polypeptides described herein can be obtained commercially, or they can be produced according to methods known in the art.

The probes can be attached to a solid support, such as paper, membranes, filters, chips, pins or glass slides, or any other appropriate substrate, such as those further described herein. For example, probes of genes which are up- or down-regulated in R.A. can be attached covalently or non covalently to membranes for use, e.g., in dotblots, or to solids such as to create arrays, e.g., microarrays.

4. Therapeutic Methods and Compositions for R.A.

The expression profiling results described in the Examples indicated that certain genes are expressed at higher levels and certain genes are expressed at lower levels in cells of R.A. patients relative to their expression in normal counterpart cells. For example, SOCS3 (CISH3); RAGE (AGER); LST-1 (LY117); SAA 1-3; HMG-1; S100 A8, A9, and A12; SLPI; GILZ; PTPN-18; GADD-45A and B; Legumain (PRSCl); FST1; Lcn2; GPI; SpiL; and TSG-6 are over-expressed in patients relative to controls. Exemplary genes which are down-regulated include CMAK2B, PLA2G2A, GBAS and SOX15. Accordingly, reducing the expression of one or more of genes that are up-regulated and/or increasing the expression of one or more genes which are down-regulated in diseased cells may provide a method of treatment of R.A. Genes, whose normalization of expression improves R.A. can be identified according to methods known in the art, some of which are set forth below. Accordingly, the invention provides compositions for these therapeutic methods, e.g., compositions comprising isolated polypeptides encoded by a gene selected from the group consisting of SOCS3 (CISH3); RAGE (AGER); LST-1 (LY117); SAA 1-3; HMG-1; S100 A8, A9, and A12; SLPI; GILZ; PTPN-18; GADD-45A and B; Legumain (PRSC1); FST1; Lcn2; GPI; SpiL; and TSG-6; nucleic acids encoding such; plasmids, vectors and host cell comprising these isolated nucleic acids; methods for making a polypeptide; and methods for identifying compounds which modulate gene expression of the genes or the activity of a polypeptide encoded by the genes.

4.1. Methods for Determining Whether Modulation of the Expression of a Gene Improves R.A.

In one embodiment, the effect of up- or down-regulating the level of expression of a gene which is down- or up-regulated, respectively, in a cell characteristic of R.A. is determined by phenotypic analysis of the cell, in particular by determining whether the cell adopts a phenotype that is more reminiscent of that of a normal cell than that of a cell characteristic of R.A.

In another preferred embodiment, the effect on the cell is determined by measuring the level of expression of one or more genes which are up- or down-regulated in R.A., and preferably at least about 10, or at least about 100 genes characteristic of R.A. In a preferred embodiment, the level of expression of a gene is modulated, and the level of expression of at least one gene characteristic of R.A. is determined, e.g., by using a microarray having probes to the one or more genes. If the normalization of expression of the gene results in at least some normalization of the gene expression profile in the diseased cell, then normalizing the expression of the gene in a subject having R.A. is expected to improve R.A. The term “normalization of the expression of a gene in a diseased cell” refers to bringing the level of expression of that gene in the diseased cell to a level that is similar to that in the corresponding normal cell. “Normalization of the gene expression profile in a diseased cell” refers to bringing the expression profile in a diseased cell essentially to that in the corresponding non-diseased cell. If, however, the normalization of expression of the gene does not result in at least some normalization of the gene expression profile in the diseased cell, normalizing the expression of the gene in a subject having R.A. is not expected to improve R.A. In certain embodiments, the expression level of two or more genes which are up- or down-regulated in R.A. is modulated and the effect on the diseased cell is determined.

A preferred cell for use in these assays is a cell characteristic of R.A. that can be obtained from a subject and, e.g., established as a primary cell culture. The cell can be immortalized by methods known in the art, e.g., by expression of an oncogene or large T antigen of SV40. Alternatively, cell lines corresponding to such a diseased cell can be used. Examples include RAW cells and THP1 cells. However, prior to using such cell lines, it may be preferably to confirm that the gene expression profile of the cell line corresponds essentially to that of a cell characteristic of R.A. This can be done as described in details herein.

Modulating the expression of a gene in a cell can be achieved, e.g., by contacting the cell with an agent that increases the level of expression of the gene or the activity of the polypeptide encoded by the gene. Increasing the level of a polypeptide in a cell can also be achieved by transfecting the cell, transiently or stably, with a nucleic acid encoding the polypeptide. Decreasing the expression of a gene in a cell can be achieved by inhibiting transcription or translation of the gene or RNA, e.g., by introducing antisense nucleic acids, ribozymes or siRNAs into the cells, or by inhibiting the activity of the polypeptide encoded by the gene, e.g., by using antibodies or dominant negative mutants. These methods are further described below in the context of therapeutic methods.

A nucleic acid encoding a particular polypeptide can be obtained, e.g., by RT-PCR from a cell that is known to express the gene. Primers for the RT-PCR can be derived from the nucleotide sequence of the gene encoding the polypeptide. The nucleotide sequence of the gene is available, e.g., in GenBank or in the publications. GenBank Accession numbers of the genes listed in Tables 1-5 are provided in the tables. Amplified DNA can then be inserted into an expression vector, according to methods known in the art and transfected into diseased cells of R.A. In a control experiment, normal counterpart cells can also be transfected. The level of expression of the polypeptide in the transfected cells can be determined, e.g., by electrophoresis and staining of the gel or by Western blot using an a agent that binds the polypeptide, e.g., an antibody. The level of expression of one or more genes which are up- or down-regulated in R.A. can then be determined in the transfected cells having elevated levels of the polypeptide. In a preferred embodiment, the level of expression is determined by using a microarray. For example, RNA is extracted from the transfected cells, and used as target DNA for hybridization to a microarray, as further described herein.

These assays will allow the identification of genes which are up- or down-regulated in R.A. which can be used as therapeutic targets for developing therapeutics for R.A.

4.2. Therapeutic Methods

4.2.1. Methods for Reducing Expression of Gene in the Cells of a Patient

Genes which are up-regulated in R.A. may be used as therapeutic targets for treating R.A. For example, it may be possible to treat R.A. by decreasing the level of the polypeptide in the diseased cells.

(i) Antisense Nucleic Acids

One method for decreasing the level of expression of a gene is to introduce into the cell antisense molecules which are complementary to at least a portion of the gene or RNA of the gene. An “antisense” nucleic acid as used herein refers to a nucleic acid capable of hybridizing to a sequence-specific (e.g., non-poly A) portion of the target RNA, for example its translation initiation region, by virtue of some sequence complementarity to a coding and/or non-coding region. The antisense nucleic acids of the invention can be oligonucleotides that are double-stranded or single-stranded, RNA or DNA or a modification or derivative thereof, which can be directly administered in a controllable manner to a cell or which can be produced intracellularly by transcription of exogenous, introduced sequences in controllable quantities sufficient to perturb translation of the target RNA.

Preferably, antisense nucleic acids are of at least six nucleotides and are preferably oligonucleotides (ranging from 6 to about 200 oligonucleotides). In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof, single-stranded or double-stranded. The oligonucleotide can be modified at the base moiety, sugar moiety, or phosphate backbone. The oligonucleotide may include other appending groups such as peptides, or agents facilitating transport across the cell membrane (see, e.g., Letsinger et al., 1989, Proc. Natl. Acad. Sci. U.S.A. 86: 6553-6556; Lemaitre et al., 1987, Proc. Natl. Acad. Sci. 84: 648-652: PCT Publication No. WO 88/09810, published Dec. 15, 1988), hybridization-triggered cleavage agents (see, e.g., Krol et al., 1988, BioTechniques 6: 958-976) or intercalating agents (see, e.g., Zon, 1988, Pharm. Res. 5: 539-549).

In a preferred aspect of the invention, an antisense oligonucleotide is provided, preferably as single-stranded DNA. The oligonucleotide may be modified at any position on its structure with constituents generally known in the art. For example, the antisense oligonucleotides may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

In another embodiment, the oligonucleotide comprises at least one modified sugar moiety selected from the group including, but not limited to, arabinose, 2-fluoroarabinose, xylulose, and hexose.

In yet another embodiment, the oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof. In yet another embodiment, the oligonucleotide is a 2-α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641).

The oligonucleotide may be conjugated to another molecule, e.g., a peptide, hybridization triggered cross-linking agent transport agent, hybridization-triggered cleavage agent, etc. An antisense molecule can be a “peptide nucleic acid” (PNA). PNA refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

The antisense nucleic acids of the invention comprise a sequence complementary to at least a portion of a target RNA species. However, absolute complementarity, although preferred, is not required. A sequence “complementary to at least a portion of an RNA,” as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a target RNA it may contain and still form a stable duplex (or triplex, as the case may be). One skilled in the art can ascertain a tolerable degree of mismatch by use of standard procedures to determine the melting point of the hybridized complex. The amount of antisense nucleic acid that will be effective in the inhibiting translation of the target RNA can be determined by standard assay techniques.

The synthesized antisense oligonucleotides can then be administered to a cell in a controlled manner. For example, the antisense oligonucleotides can be placed in the growth environment of the cell at controlled levels where they may be taken up by the cell. The uptake of the antisense oligonucleotides can be assisted by use of methods well known in the art.

In an alternative embodiment, the antisense nucleic acids of the invention are controllably expressed intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequences encoding the antisense RNAs can be by any promoter known in the art to act in a cell of interest. Such promoters can be inducible or constitutive. Most preferably, promoters are controllable or inducible by the administration of an exogenous moiety in order to achieve controlled expression of the antisense oligonucleotide. Such controllable promoters include the Tet promoter. Other usable promoters for mammalian cells include, but are not limited to: the SV40 early promoter region (Bernoist and Chambon, 1981, Nature 290: 304-310), the promoter contained in the 3′ long terminal repeat of Rous sarcoma virus (Yamamoto et al., 1980, Cell 22: 787-797), the herpes thymidine kinase promoter (Wagner et al., 1981, Proc. Natl. Acad. Sci. U.S.A. 78: 1441-1445), the regulatory sequences of the metallothionein gene (Brinster et al., 1982, Nature 296: 39-42), etc.

Antisense therapy for a variety of cancers is in clinical phase and has been discussed extensively in the literature. Reed reviewed antisense therapy directed at the Bcl-2 gene in tumors; gene transfer-mediated overexpression of Bcl-2 in tumor cell lines conferred resistance to many types of cancer drugs. (Reed, J. C., N.C.I. (1997) 89:988-990). The potential for clinical development of antisense inhibitors of ras is discussed by Cowsert, L. M., Anti-Cancer Drug Design (1997) 12:359-371. Additional important antisense targets include leukemia (Geurtz, A. M., Anti-Cancer Drug Design (1997) 12:341-358); human C-ref kinase (Monia, B. P., Anti-Cancer Drug Design (1997) 12:327-339); and protein kinase C (McGraw et al., Anti-Cancer Drug Design (1997) 12:315-326.

(ii) Ribozymes

In another embodiment, the level of a particular mRNA or polypeptide in a cell is reduced by introduction of a ribozyme into the cell or nucleic acid encoding such. Ribozyme molecules designed to catalytically cleave mRNA transcripts can also be introduced into, or expressed, in cells to inhibit expression of the gene (see, e.g., Sarver et al., 1990, Science 247:1222-1225 and U.S. Pat. No. 5,093,246). One commonly used ribozyme motif is the -hammerhead, for which the substrate sequence requirements are minimal. Design of the hammerhead ribozyme is disclosed in Usman et al., Current Opin. Struct. Biol. (1996) 6:527-533. Usman also discusses the therapeutic uses of ribozymes. Ribozymes can also be prepared and used as described in Long et al., FASEB J. (1993) 7:25; Symons, Ann. Rev. Biochem. (1992) 61:641; Perrotta et al., Biochem. (1992) 31:16-17; Ojwang et al., Proc. Natl. Acad. Sci. (USA) (1992) 89:10802-10806; and U.S. Pat. No. 5,254,678. Ribozyme cleavage of HIV-I RNA is described in U.S. Pat. No. 5,144,019; methods of cleaving RNA using ribozymes is described in U.S. Pat. No. 5,116,742; and methods for increasing the specificity of ribozymes are described in U.S. Pat. No. 5,225,337 and Koizumi et al., Nucleic Acid Res. (1989) 17:7059-7071. Preparation and use of ribozyme fragments in a hammerhead structure are also described by Koizumi et al., Nucleic Acids Res. (1989) 17:7059-7071. Preparation and use of ribozyme fragments in a hairpin structure are described by Chowrira and Burke, Nucleic Acids Res. (1992) 20:2835. Ribozymes can also be made by rolling transcription as described in Daubendiek and Kool, Nat. Biotechnol. (1997) 15(3):273-277.

(iii) siRNAs

Another method for decreasing or blocking gene expression is by introducing double stranded small interfering RNAs (siRNAs), which mediate sequence specific mRNA degradation. RNA interference (RNAi) is the process of sequence-specific, post-transcriptional gene silencing in animals and plants, initiated by double-stranded RNA (dsRNA) that is homologous in sequence to the silenced gene. In vivo, long dsRNA is cleaved by ribonuclease III to generate 21- and 22-nucleotide siRNAs. It has been shown that 21-nucleotide siRNA duplexes specifically suppress expression of endogenous and heterologous genes in different mammalian cell lines, including human embryonic kidney (293) and HeLa cells (Elbashir et al. Nature 2001 ;411(6836):494-8).

(iv) Triplex Formation

Gene expression can be reduced by targeting deoxyribonucleotide sequences complementary to the regulatory region of the target gene (i.e., the gene promoter and/or enhancers) to form triple helical structures that prevent transcription of the gene in target cells in the body. (See generally, Helene, C. 1991, Anticancer Drug Des., 6(6):569-84; Helene, C., et al., 1992, Ann, N.Y. Accad. Sci., 660:27-36; and Maher, L. J., 1992, Bioassays 14(12):807-15).

(v) Aptamers

In a further embodiment, RNA aptamers can be introduced into or expressed in a cell. RNA aptamers are specific RNA ligands for proteins, such as for Tat and Rev RNA (Good et al., 1997, Gene Therapy 4: 45-54) that can specifically inhibit their translation.

(vi) Dominant Negative Mutants

Another method of decreasing the biological activity of a polypeptide is by introducing into the cell a dominant negative mutant. A dominant negative mutant polypeptide will interact with a molecule with which the polypeptide normally interacts, thereby competing for the molecule, but since it is biologically inactive, it will inhibit the biological activity of the polypeptide. A dominant negative mutant can be created by mutating the substrate-binding domain, the catalytic domain, or a cellular localization domain of the polypeptide. Preferably, the mutant polypeptide will be overproduced. Point mutations are made that have such an effect. In addition, fusion of different polypeptides of various lengths to the terminus of a protein can yield dominant negative mutants. General strategies are available for making dominant negative mutants. See Herskowitz, Nature (1987) 329:219-222.

(vi) Use of Agents Inhibiting Transcription or Polypeptide Activity

In another embodiment, a compound decreasing the expression of the gene of interest or the activity of the polypeptide is administered to a subject having R.A., such that the level of the polypeptide in the diseased cells decreases, and the disease is improved. Compounds may be known in the art or can be identified as further described herein.

4.2.2. Methods for Increasing the Expression of a Protein in Cells of a Patient

Genes which are down-regulated in R.A. may be used as therapeutic targets for treating R.A. For example, it may be possible to treat R.A. by increasing the level of the polypeptide in the diseased cells.

(i) Administration of a Nucleic Acid Encoding a Polypeptide of Interest to a Subject

In one embodiment, a nucleic acid encoding a polypeptide of interest, or an equivalent thereof, such as a functionally active fragment of the polypeptide, is administered to a subject, such that the nucleic acid arrives at the site of the diseased cells, traverses the cell membrane and is expressed in the diseased cell.

A nucleic acid encoding a polypeptide of interest can be obtained as described herein, e.g., by RT-PCR, or from publicly available DNA clones. It may not be necessary to express the full length polypeptide in a cell of a subject, and a functional fragment thereof may be sufficient. Similarly, it is not necessary to express a polypeptide having an amino acid sequence that is identical to that of the wild-type polypeptide. Certain amino acid deletions, additions and substitutions are permitted, provided that the polypeptide retains most of its biological activity. For example, it is expected that polypeptides having conservative amino acid substitutions will have the same activity as the polypeptide. Polypeptides that are shorter or longer than the wild-type polypeptide or which contain from one to 20 amino acid deletions, insertions or substitutions and which have a biological activity that is essentially identical to that of the wild-type polypeptide are referred to herein as “equivalents of the polypeptide.” Equivalent polypeptides also include polypeptides having an amino acid sequence which is at least 80%, preferably at least about 90%, even more preferably at least about 95% and most preferably at least 98% identical or similar to the amino acid sequence of the wild-type polypeptide.

Determining which portion of the polypeptide is sufficient for improving R.A. or which polypeptides derived from the polypeptide are “equivalents” which can be used for treating R.A., can be done in in vitro assays. For example, expression plasmids encoding various portions of the polypeptide can be transfected into cells, e.g., diseased cells of R.A., and the effect of the expression of the portion of the polypeptide in the cells can be determined, e.g., by visual inspection of the phenotype of the cell or by obtaining the expression profile of the cell, as further described herein.

Any means for the introduction of polynucleotides into mammals, human or non-human, may be adapted to the practice of this invention for the delivery of the various constructs of the invention into the intended recipient. In one embodiment of the invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject (see below). In a preferred method of the invention, the DNA constructs are delivered using viral vectors. The transgene may be incorporated into any of a variety of viral vectors useful in gene therapy, such as recombinant retroviruses, adenovirus, adeno-associated virus (AAV), and herpes simplex virus-1, or recombinant bacterial or eukaryotic plasmids. While various viral vectors may be used in the practice of this invention, AAV- and adenovirus-based approaches are of particular interest. Such vectors are generally understood to be the recombinant gene delivery system of choice for the transfer of exogenous genes in vivo, particularly into humans.

It is possible to limit the infection spectrum of viruses by modifying the viral packaging proteins on the surface of the viral particle (see, for example PCT publications WO93/25234, WO94/06920, and WO94/11524). For instance, strategies for the modification of the infection spectrum of viral vectors include: coupling antibodies specific for cell surface antigens to envelope protein (Roux et al., (1989) PNAS USA 86:9079-9083; Julan et al., (1992) J. Gen Virol 73:3251-3255; and Goud et al., (1983) Virology 163:251-254); or coupling cell surface ligands to the viral envelope proteins (Neda et al., (1991) J. Biol. Chem. 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, and can also be used to convert an ecotropic vector in to an amphotropic vector.

The expression of a polypeptide of interest or equivalent thereof in cells of a patient to which a nucleic acid encoding the polypeptide was administered can be determined, e.g., by obtaining a sample of the cells of the patient and determining the level of the polypeptide in the sample, relative to a control sample. The successful administration to a patient and expression of the polypeptide or an equivalent thereof in the cells of the patient can be monitored by determining the expression of at least one gene characteristic of R.A., and preferably by determining an expression profile including most of the genes which are up- or down-regulated in R.A., as described herein.

(ii) Administration of a Polypeptide of Interest to a Subject

In another embodiment, a polypeptide of interest, or an equivalent thereof, e.g., a functional fragment thereof, is administered to the subject such that it reaches the diseased cells of R.A., and traverses the cellular membrane. Polypeptides can be synthesized in prokaryotes or eukaryotes or cells thereof and purified according to methods known in the art. For example, recombinant polypeptides can be synthesized in human cells, mouse cells, rat cells, insect cells, yeast cells, and plant cells. Polypeptides can also be synthesized in cell free extracts, e.g., reticulocyte lysates or wheat germ extracts. Purification of proteins can be done by various methods, e.g., chromatographic methods (see, e.g., Robert K Scopes “Protein Purification: Principles and Practice” Third Ed. Springer-Verlag, N.Y. 1994). In one embodiment, the polypeptide is produced as a fusion polypeptide comprising an epitope tag consisting of about six consecutive histidine residues. The fusion polypeptide can then be purified on a Ni⁺⁺ column. By inserting a protease site between the tag and the polypeptide, the tag can be removed after purification of the peptide on the Ni⁺⁺ column. These methods are well known in the art and commercial vectors and affinity matrices are commercially available.

Administration of polypeptides can be done by mixing them with liposomes, as described above. The surface of the liposomes can be modified by adding molecules that will target the liposome to the desired physiological location.

In one embodiment, a polypeptide is modified so that its rate of traversing the cellular membrane is increased. For example, the polypeptide can be fused to a second peptide which promotes “transcytosis,” e.g., uptake of the peptide by cells. In one embodiment, the peptide is a -portion of the HIV transactivator (TAT) protein, such as the fragment corresponding to residues 37-62 or 48-60 of TAT, portions which are rapidly taken up by cell in vitro (Green and Loewenstein, (1989) Cell 55:1179-1188). In another embodiment, the internalizing peptide is derived from the Drosophila antennapedia protein, or homologs thereof. The 60 amino acid long homeodomain of the homeo-protein antennapedia has been demonstrated to translocate through biological membranes and can facilitate the translocation of heterologous polypeptides to which it is couples. Thus, polypeptides can be fused to a peptide consisting of about amino acids 42-58 of Drosophila antennapedia or shorter fragments for transcytosis. See for example Derossi et al. (1996) J Biol Chem 271:18188-18193; Derossi et al. (1994) J Biol Chem 269:10444-10450; and Perez et al. (1992) J Cell Sci 102:717-722.

(iii) Use of Agents Stimulating Transcription or Polypeptide Activity

In another embodiment, a pharmaceutical composition comprising a compound that stimulates the level of expression of a gene of interest or the activity of the polypeptide in a cell is administered to a subject, such that the level of expression of the gene in the diseased cells is increased or even restored, and R.A. is improving in the subject. Compounds may be known in the art or can be identified as further described herein.

4.3. Drug Design and Discovery of Therapeutics

As described above, genes whose modulation of expression improve R.A. can be used as targets in drug design and discovery. For example, assays can be conducted to identify molecules that modulate the expression and or activity of genes which are up- or down-regulated in R.A.

In one embodiment, an agent which modulates the expression of a gene of interest is identified by contacting cells expressing the gene with test compounds, and monitoring the level of expression of the gene. Alternatively, compounds which modulate the expression of gene X can be identified by conducting assays using the promoter region of a gene and screening for compounds which modify binding of proteins to the promoter region. The nucleotide sequence of the promoter may be described in a publication or available in GenBank. Alternatively, the promoter region of the gene can be isolated, e.g., by screening a genomic library with a probe corresponding to the gene. Such methods are known in the art.

Inhibitors of the polypeptide can also be agents which bind to the polypeptide, and thereby prevent it from functioning normally, or which degrades or causes the polypeptide to be degraded. For example, such an agent can be an antibody or derivative thereof which interacts specifically with the polypeptide. Preferred antibodies are monoclonal antibodies, humanized antibodies, human antibodies, and single chain antibodies. Such antibodies can be prepared and tested as known in the art.

If a polypeptide of interest binds to another polypeptide, drugs can be developed which modulate the activity of the polypeptide by modulating its binding to the other polypeptide (referred to herein as “binding partner”). Cell-free assays can be used to identify compounds which are capable of interacting with the polypeptide or binding partner, to thereby modify the activity of the polypeptide or binding partner. Such a compound can, e.g., modify the structure of the polypeptide or binding partner and thereby effect its activity. Cell-free assays can also be used to identify compounds which modulate the interaction between the polypeptide and a binding partner. In a preferred embodiment, cell-free assays for identifying such compounds consist essentially in a reaction mixture containing the polypeptide and a test compound or a library of test compounds in the presence or absence of a binding partner. A test compound can be, e.g., a derivative of a binding partner, e.g., a biologically inactive peptide, or a small molecule.

Accordingly, one exemplary screening assay of the present invention includes the steps of contacting the polypeptide or functional fragment thereof or a binding partner with a test compound or library of test compounds and detecting the formation of complexes. For detection purposes, the molecule can be labeled with a specific marker and the test compound or library of test compounds labeled with a different marker. Interaction of a test compound with a polypeptide or fragment thereof or binding partner can then be detected by determining the level of the two labels after an incubation step and a washing step. The presence of two labels after the washing step is indicative of an interaction.

An interaction between molecules can also be identified by using real-time BIA (Biomolecular Interaction Analysis, Pharmacia Biosensor AB) which detects surface plasmon resonance (SPR), an optical phenomenon. Detection depends on changes in the mass concentration of macromolecules at the biospecific interface, and does not require any labeling of interactants. In one embodiment, a library of test compounds can be immobilized on a sensor surface, e.g., which forms one wall of a micro-flow cell. A solution containing the polypeptide, functional fragment thereof, polypeptide analog or binding partner is then flown continuously over the sensor surface. A change in the resonance angle as shown on a signal recording, indicates that an interaction has occurred. This technique is further described, e.g., in BlAtechnology Handbook by Pharmacia.

Another exemplary screening assay of the present invention includes the steps of (a) forming a reaction mixture including: (i) a polypeptide of interest, (ii) a binding partner, and (iii) a test compound; and (b) detecting interaction of the polypeptide and the binding partner. The polypeptide and binding partner can be produced recombinantly, purified from a source, e.g., plasma, or chemically synthesized, as described herein. A statistically significant change (potentiation or inhibition) in the interaction of the polypeptide and binding partner in the presence of the test compound, relative to the interaction in the absence of the test compound, indicates a potential agonist (mimetic or potentiator) or antagonist (inhibitor) of the polypeptide bioactivity for the test compound. The compounds of this assay can be contacted simultaneously. Alternatively, the polypeptide can first be contacted with a test compound for an appropriate amount of time, following which the binding partner is added to the reaction mixture. The efficacy of the compound can be assessed by generating dose response curves from data obtained using various concentrations of the test compound. Moreover, a control assay can also be performed to provide a baseline for comparison. In the control assay, isolated and purified polypeptide or binding partner is added to a composition containing the binding partner or polypeptide, and the formation of a complex is quantified in the absence of the test compound.

Complex formation between a polypeptide and a binding partner may be detected by a variety of techniques. Modulation of the formation of complexes can be quantitated using, for example, detectably labeled proteins such as radiolabeled, fluorescently labeled, or enzymatically labeled polypeptides or binding partners, by immunoassay, or by chromatographic detection.

Typically, it will be desirable to immobilize either the polypeptide or its binding partner to facilitate separation of complexes from uncomplexed forms of one or both of the proteins, as well as to accommodate automation of the assay. Binding of the polypeptide to a binding partner, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/polypeptide (GST/polypeptide) fusion proteins can be adsorbed onto glutathione sepharose beads (Sigma Chemical, St. Louis, Mo.) or glutathione derivatized microtitre plates, which are then combined with the binding partner, e.g. an ³⁵S-labeled binding partner, and the test compound, and the mixture incubated under conditions conducive to complex formation, e.g. at physiological conditions for salt and pH, though slightly more stringent conditions may be desired. Following incubation, the beads are washed to remove any unbound label, and the matrix immobilized and radiolabel determined directly (e.g. beads placed in scintilant), or in the supernatant after the complexes are subsequently dissociated. Alternatively, the complexes can be dissociated from the matrix, separated by SDS-PAGE, and the level of the polypeptide or binding partner found in the bead fraction quantitated from the gel using standard electrophoretic techniques such as described in the appended examples.

Other techniques for immobilizing proteins on matrices are also available for use in the subject assay. For instance, either the polypeptide or its cognate binding partner can be immobilized utilizing conjugation of biotin and streptavidin. For instance, biotinylated polypeptide molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with the polypeptide can be derivatized to the wells of the plate, and the polypeptide trapped in the wells by antibody conjugation. As above, preparations of a binding partner and a test compound are incubated in the polypeptide X presenting wells of the plate, and the amount of complex trapped in the well can be quantitated. Exemplary methods for detecting such complexes, in addition to those described above for the GST-immobilized complexes, include immunodetection of complexes using antibodies reactive with the binding partner, or which are reactive with the polypeptide and compete with the binding partner; as well as enzyme-linked assays which rely on detecting an enzymatic activity associated with the binding partner, either intrinsic or extrinsic activity. In the instance of the latter, the enzyme can be chemically conjugated or provided as a fusion protein with the binding partner. To illustrate, the binding partner can be chemically cross-linked or genetically fused with horseradish peroxidase, and the amount of polypeptide trapped in the complex can be assessed with a chromogenic substrate of the enzyme, e.g. 3,3′-diamino-benzadine terahydrochloride or 4-chloro-1-napthol. Likewise, a fusion protein comprising the polypeptide and glutathione-S-transferase can be provided, and complex formation quantitated by detecting the GST activity using 1-chloro-2,4-dinitrobenzene (Habig et al (1974) J Biol Chem 249:7130).

For processes that rely on immunodetection for quantitating one of the proteins trapped in the complex, antibodies against the protein can be used. Alternatively, the protein to be detected in the complex can be “epitope tagged” in the form of a fusion protein which includes, in addition to the polypeptide sequence, a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharmacia, N.J.).

4.4. Drug Design Using Microarrays

The invention also provides methods for designing and optimizing drugs for R.A., e.g., those which have been identified as described herein. In one embodiment, compounds are screened by comparing the expression level of one or more genes which are up- or down-regulated in R.A. in a cell characteristic of R.A. treated with a drug relative to their expression in a reference cell. In an even more preferred embodiment, the expression level of the genes is determined using microarrays, by comparing the gene expression profile of a cell treated the with a test compound with the gene expression profile of a normal counterpart cell (a “reference profile”). Optionally the expression profile is also compared to that of a cell characteristic of R.A. The comparisons are preferably done by introducing the gene expression profile data of the cell treated with the drug into a computer system comprising reference gene expression profiles which are stored in a computer readable form, using appropriate aglorithms. Test compounds will be screened for those which alter the level of expression of genes which are up- or down-regulated in R.A., so as to bring them to a level that is similar to that in a cell of the same type as a cell characteristic of R.A. are. Such compounds, i.e., compounds which are capable of normalizing the expression of at least about 10%, preferably at least about 20%, 50%, 70%, 80% or 90% of the genes which are up- or down-regulated in R.A., are candidate therapeutics.

The efficacy of the compounds can then be tested in additional in vitro assays and in vivo, in animal models. Animal models of R.A. include the collagen-induced arthritis mouse model (see Examples). The test compound is administered to the test animal and one or more symptoms of the disease are monitored for improvement of the condition of the animal. Expression of one or more genes which are up- or down-regulated in R.A. can also be measured before and after administration of the test compound to the animal. A normalization of the expression of one or more of these genes is indicative of the efficiency of the compound for treating R.A. in the animal.

The toxicity of the candidate therapeutic compound, such as resulting from a stress-related response, can be evaluated, e.g., by determining whether it induces the expression of genes known to be associated with a toxic response. Expression of such toxicity related genes may be determined in different cell types, preferably those that are known to express the genes. In a preferred method, microarrays are used for detecting changes in gene expression of genes known to be associated with a toxic response. Changes in gene expression may be a more sensitive marker of human toxicity than routine preclinical safety studies. It was shown, e.g., that a drug which was found not be to toxic in laboratory animals was toxic when administered to humans. When gene profiling was studied in cells contacted with the drug, however, it was found that a gene, whose expression is known to correlate to liver toxicity, was expressed (see below).

Such microarrays will comprise genes which are modulated in response to toxicity or stress. An exemplary array that can be used for that purpose is the Affymetrix Rat Toxicology U34 array, which contains probes of the following genes: metabolism enzymes, e.g., CYP450s, acetyltransferases, and sulfotransferases; growth factors and their receptors, e.g., IGFs, interleukins, NGTs, TGFs, and VEGT; kinases and phosphatases, e.g, lipid kinases, MAFKs, and stress-activated kinases; nuclear receptors, e.g., retinoic acid, retinoid X and PPARs; transcription factors, e.g., oncogenes, STATs, NF-kB, and zinc finger proteins; apoptosis genes, e.g., Bcl-2 genes, Bad, Bax, Caspases and Fas; stress response genes, e.g., heat-shock proteins and drug transporters; membrane proteins, e.g., gap-junction proteins and selectins; and cell-cycle regulators, e.g., cyclins and cyclin-associated proteins. Other genes included in the microarrays are only known because they contain the nucleotide sequence of an EST and because they have a connection with toxicity.

In one embodiment, a drug of interest is incubated with a cell, e.g., a cell in culture, the RNA is extracted, and expression of genes is analyzed with an array containing genes which have been shown to be up- or down-regulated in response to certain toxins. The results of the hybridization are then compared to databases containing expression levels of genes in response to certain known toxins in certain organisms. For example, the GeneLogic ToxExpress™ database can be used for that purpose. The information in this database was obtained in least in part from the use of the Affymetrix GeneChip® rat and human probe arrays with samples treated in vivo or in vitro with known toxins. The database contains levels of expression of liver genes in response to known liver toxins. These data were obtained by treating liver samples from rats treated in vivo with known toxins, and comparing the level of expression of numerous genes with that in rat or human primary hepatocytes treated in vitro with the same toxin. Data profiles can be retrieved and analyzed with the GeneExpress™ database tools, which are designed for complex data management and analysis. As indicated on the Affymetrix (Santa Clara, Calif.) website, the GeneLogic, Inc. (Gaithersburg, Md.) has preformed proof of concept studies showing the changes in gene expression levels can predict toxic events that were not identified by routine preclinical safety testing. GeneLogic tested a drug that had shown no evidence of liver toxicity in rats, but that later showed toxicity in humans. The hybridization results using the Affymetrix GeneChip® and GeneExpress™ tools showed that the drug caused abnormal elevations of alanine aminotransferase (ALT), which indicates liver injury, in half of the patients who had used the drug.

In one embodiment of the invention, the drug of interest is administered to an animal, such as a mouse or a rat, at different doses. As negative controls, animals are administered the vehicle alone, e.g., buffer or water. Positive controls can consist of animals treated with drugs known to be toxic. The animals can then be sacrificed at different times, e.g., at 3, 6, and 24 hours, after administration of the drug, vehicle alone or positive control drug, mRNA extracted from a sample of their liver; and the mRNA analyzed using arrays containing nucleic acids of genes which are likely to be indicative of toxicity, e.g., the Affymetrix Rat Toxicology U34 assay. The hybridization results can then be analyzed using computer programs and databases, as described above.

In addition, toxicity of a drug in a subject can be predicted based on the alleles of drug metabolizing genes that are present in a subject. Accordingly, it is known that certain enzymes, e.g., cytochrome p450 enzymes, i.e., CYP450, metabolize drugs, and thereby may render drugs which are innocuous in certain subjects, toxic in others. A commercially available array containing probes of different alleles of such drug metabolizing genes can be obtained, e.g., from Affymetrix (Santa Clara, Calif.), under the name of GeneChip® CYP450 assay.

Thus, a drug for R.A. identified as described herein can be optimized by reducing any toxicity it may have. Compounds can be derivatized in vitro using known chemical methods and tested for expression of toxicity related genes. The derivatized compounds must also be retested for normalization of expression levels of genes which are up- or down-regulated in R.A. For example, the derivatized compounds can be incubated with diseased cells of R.A., and the gene expression profile determined using microarrays. Thus, incubating cells with derivatized compounds and measuring gene expression levels with a microarray that contains the genes which are up- or down-regulated in R.A. and a microarray containing toxicity related genes, compounds which are effective in treating R.A. and which are not toxic can be developed. Such compounds can further be tested in animal models as described above.

In another embodiment of the invention, a drug is developed by rational drug design, i.e., it is designed or identified based on information stored in computer readable form and analyzed by algorithms. More and more databases of expression profiles are currently being established, numerous ones being publicly available. By screening such databases for the description of drugs affecting the expression of at least some of the genes which are up- or down-regulated in R.A. in a manner similar to the change in gene expression profile from a cell characteristic of R.A. to that of a normal counterpart cell, compounds can be identified which normalize gene expression in a cell characteristic of R.A. Derivatives and analogues of such compounds can then be synthesized to optimize the activity of the compound, and tested and optimized as described above.

Compounds identified by the methods described above are within the scope of the invention. Compositions comprising such compounds, in particular, compositions comprising a pharmaceutically efficient amount of the drug in a pharmaceutically acceptable carrier are also provided. Certain compositions comprise one or more active compounds for treating R.A.

The invention also provides methods for designing therapeutics for treating diseases that are different from R.A., but related thereto. Related diseases may in fact have a gene expression profile, which even though not identical to that of R.A., will show some homology, so that drugs for treating R.A. can be used for treating the related disease or for starting the research of compounds for treating the related disease. A compound for treating R.A. can be derivatized and tested as further described herein.

4.5. Exemplary Therapeutic Compositions

Therapeutic compositions include the compounds described herein, e.g., in the context of therapeutic treatments of R.A. Therapeutic compositions may comprise one or more nucleic acids encoding a polypeptide characteristic of R.A., or equivalents thereof. The nucleic acids may be in expression vectors, e.g., viral vectors. Other compositions comprise one or more polypeptides characteristic of R.A., or equivalents thereof. Yet other compositions comprise nucleic acids encoding antisense RNA, or ribozymes, siRNAs or RNA aptamers. Also within the scope of the invention are compositions comprising compounds identified by the methods described herein. The compositions may comprise pharmaceutically acceptable excipients, and may be contained in a device for their administration, e.g., a syringe.

4.6. Administration of Compounds and Compositions of the Invention

In a preferred embodiment, the invention provides a method for treating a subject having R.A., comprising administering to the subject a therapeutically effective amount of a pharmaceutical composition comprising a compound of the invention.

4.6.1. Effective Dose

Compounds of the invention refer to small molecules, polypeptides, peptide mimetics, nucleic acids or any other molecule identified as potentially useful for treating R.A.

Toxicity and therapeutic efficacy of compounds can be determined by standard pharmaceutical procedures in cell cultures or experimental animals, e.g., for determining the LD50 (The Dose Lethal To 50% Of The Population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in ordei to minimize potential damage to healthy cells and, thereby, reduce side effects.

Data obtained from cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

4.6.2. Formulation

Pharmaceutical compositions for use in accordance with the present invention may be formulated in conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral or rectal administration. In one embodiment, the compound is administered locally, at the site where the diseased cells are present, i.e., in the blood or in a joint.

The compounds of the invention can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets, lozanges, or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Administration, e.g., systemic administration, can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the compounds of the invention can be formulated into ointments, salves, gels, or creams as generally known in the art. A wash solution can be used locally to treat an injury or inflammation to accelerate healing.

In clinical settings, a gene delivery system for a gene of interest can be introduced into a patient by any of a number of methods, each of which is familiar in the art. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the subject or animal being quite localized. For example, the gene delivery vehicle can be introduced by catheter (see U.S. Pat. No. 5,328,470) or by stereotactic injection (e.g., Chen et al. (1994) PNAS 91: 3054-3057). A nucleic acid, such as one encoding a polypeptide of interest or homologue thereof can be delivered in a gene therapy construct by electroporation using techniques described, for example, by Dev et al. ((1994) Cancer Treat Rev 20:105-115). Gene therapy can be conducted in vivo or ex vivo.

The pharmaceutical preparation of the gene therapy construct or compound of the invention can consist essentially of the gene delivery system in an acceptable diluent, or can comprise a slow release matrix in which the gene delivery vehicle or compound is imbedded. Alternatively, where the complete gene delivery system can be produced intact from recombinant cells, e.g., retroviral vectors, the pharmaceutical preparation can comprise one or more cells which produce the gene delivery system.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

4. Exemplary Kits

The invention further provides kits for determining the expression level of genes characteristic of disease B. The kits may be useful for identifying subjects that are predisposed to developing R.A. or who have R.A., as well as for identifying and validating therapeutics for R.A. In one embodiment, the kit comprises a computer readable medium on which is stored one or more gene expression profiles of diseased cells of R.A., or at least values representing levels of expression of one or more genes which are up- or down-regulated in R.A. in a diseased cell. The computer readable medium can also comprise gene expression profiles of counterpart normal cells, diseased cells treated with a drug, and any other gene expression profile described herein. The kit can comprise expression profile analysis software capable of being loaded into the memory of a computer system.

A kit can comprise a microarray comprising probes of genes which are up- or down-regulated in R.A. A kit can comprise one or more probes or primers for detecting the expression level of one or more genes which are up- or down-regulated in R.A. and/or a solid support on which probes attached and which can be used for detecting expression of one or more genes which are up- or down-regulated in R.A. in a sample. A kit may further comprise nucleic acid controls, buffers, and instructions for use.

Other kits provide compositions for treating R.A. For example, a kit can also comprise one or more nucleic acids corresponding to one or more genes which are up- or down-regulated in R.A., e.g., for use in treating a patient having R.A. The nucleic acids can be included in a plasmid or a vector, e.g., a viral vector. Other kits comprise a polypeptide encoded by a gene characteristic of R.A. or an antibody to a polypeptide. Yet other kits comprise compounds identified herein as agonists or antagonists of genes which are up- or down-regulated in R.A. The compositions may be pharmaceutical compositions comprising a pharmaceutically acceptable excipient.

The present invention is further illustrated by the following examples which should not be construed as limiting in any way. The contents of all cited references including literature references, issued patents, published and non published patent applications as cited throughout this application are hereby expressly incorporated by reference.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); (R. I. Freshney, Alan R. Liss, Inc., 1987); Immobilized Cells And Enzymes (RL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); , Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986) (Cold Spring Harbor Laboratory Press, Cold Spring Harbor, N.Y., 1986).

### EXAMPLES

### Example 1

### Identification of Genes that are Up- or Down-Regulated in Patients Having Rheumatoid Arthritis

This Example describes the identification of several genes which are up- or downregulated in peripheral blood mononuclear cells (PBMCs) of subjects having rheumatoid arthritis (R.A.) relative to expression in PBMCs of normal subjects.

PMBCs were isolated form 9 patients with R.A. and 13 normal volunteers as follows. Eight mls of blood were drawn into a CPT Vacutainer tube which was inverted several times. The tube was centrifuged at 1500× g (2700 rpm) in a swinging bucket rotor at room temperature. The serum was removed and PBMCs were transferred to a 15 ml conical centrifuge tube. The cells were washed with the addition of phosphate buffered saline (PBS) and centrifuged at 450 g (1200 rpm) for 5 minutes. The supernatant was discarded and the wash procedure was repeated once more. After removal of the supernatant, total RNA was isolated with the use of the RNeasy minikit, (Qiagen,Hidden,Germany) according to the manufacturers procedure.

RNA was analyzed on oligonucleotide arrays composed of 6,800 and 12,000 human genes (Affymetrix Hu6800 and HgU95A chip sets, respectively), as follows.

Target nucleic acid for hybridization was prepared as follows. Total RNA was prepared for hybridization by denaturing 5 μg of total RNA from PBMC's for 10 minutes at 70° C. with 100 pM T7/T24-tagged oligo-dt primer (synthesized at Genetics Institute, Cambridge, Mass.), and cooled on ice. First strand cDNA synthesis was performed under the following buffer conditions: 1× first strand buffer (Invitrogen Life Technologies, Carlsbad, Calif.), 10 mM DTT(GIBCO/Invitrogen), 500 μM of each dNTP (Invitrogen Life Technologies), 400 units of Superscript RT 11 (Invitrogen Life Technologies) and 40 units RNAse inhibitor (Ambion,Austin, Tex.). The reaction proceeded at 47° C. for 1 hour. Second strand cDNA was synthesized with the addition of the following reagents at the final concentrations listed: 1× second strand buffer (Invitrogen Life Technologies), an additional 200 μM of each dNTP (Invitrogen Life Technologies), 40 units of E. coli DNA polymerase I (Invitrogen Life Technologies), 2 units E. coli RNaseH (Invitrogen Life Technologies), and 10 units of E. coli DNA ligase. The reaction proceeded at 15.8° C. for 2 hours and during the last five minutes of this reaction 6 units of T4 DNA polymerase (New England Biolabs, Beverly, Mass.) was added. The resulting double stranded cDNA was purified with the use of BioMag carboxyl terminated particles as follows: 0.2 mg of BioMag particles (Polysciences Inc., Warrington, PA) were equilibrated by washing three times with 0.5M EDTA and resuspended at a concentration of 22.2 mg/ml in 0.5M EDTA. The double stranded cDNA reaction was diluted to a final concentration of 10% PEG/1.25M NaCl and the bead suspension was added to a final bead concentration of 0.614 mg/ml. The reaction was incubated at room temperature for 10 minutes. The cDNA/bead complexes were washed with 300μl of 70% ethanol, the ethanol was removed and the tubes were allowed to air dry. The cDNA was eluted with the addition of 20 μl of 10 mM Tris-acetate, pH 7.8, incubated for 2-5 minutes and the cDNA containing supernatant was removed. 10μl of purified double stranded cDNA was then added to an in vitro transcription (IVT) solution which contained, 1× IVT buffer (Ambion, Austin, Tex.) 5,000 units T7 RNA polymerase (Epicentre Technologies, Madison, Wis.), 3 mM GTP, 1.5 mM ATP, 1.2 mM CTP and 1.2 mM UTP (Amersham/Pharmacia,), 0.4 mM each bio-16 UTP and bio-11 CTP (Enzo Diagnostics, Farmingdale, NY), and 80 units RNase inhibitor (Ambion, Austin, Tex.). The reaction proceeded at 37° C. for 16 hours. Labeled RNA was purified with the use of an RNeasy (Qiagen). The RNA yield was quantitated by measuring absorbance at 260 nm.

Array Hybridization and Detection of Fluorescence was performed as follows. 12 μg of IVT was fragmented in 40 mM Tris-actetate, pH 8.0, 100 mM potassium acetate, and 30 mM magnesium acetate for 35 minutes at 94° C. The fragmented, labeled RNA probes were diluted in hybridization buffer at a final composition of 1× 2-N-Morpholinoethanesulfonic acid (MES (buffer (pH 6.5), 50 pM Bio948 (control biotinylated oligo that hybridizes to landmark features on the probe array (Genetics Institute,Cambridge, Mass.)), 100 μg/ml herring sperm DNA (Promega, Madison, Wis.), 500 μg/ml acetylated BSA (Invitrogen Life Technologies) and 1 μl/μg standard curve reagent (Proprietary reagent supplied by Gene Logic,Gaithersburg, Md.). This hybridization solution was pre-hybridized with two glass beads (Fisher Scientific, Pittsburgh, Pa.) at 45° C. overnight. The hybridization solution was removed to a clean tube, heated for 1-2 min at 95° C. and microcentrifuged on high for 2 minutes to pellet insoluble debris. Affymetrix oligonucleotide array cartridges (human 6800 array P/N900183 and human U95A (Affymetrix, Santa Clara, Calif.)) were pre-wet with non-stringent wash buffer (0.9M NaCl, 60 mM sodium phosphate, 6 mM EDTA and 0.01% Tween20) and incubated at 45° C. with rotation for 5-10 minutes. Buffer was removed from the Affymetrix cartridges and the arrays were hybridized with 180 μl of the hybridization solution at 45° C. rotating at 45-60 rpm overnight. After overnight incubation, the hybridization solutions were removed and the cartridges were filled with non-stringent wash buffer. The array cartridges were washed using an Affymetrix fluidics station according with 10 cycles of 2 mixes/cycle non-stringent wash buffer at 25° C. followed by 4 cycles of 15 mixes/cycle stringent wash buffer (100 mM MES, 0.1M Na⁺, 0.01% Tween20 and 0.005% antifoam). The probe array was then first stained for 10 minutes at 25° C. in SAPE solution (100 mM MES, 1M Na⁺, 0.05% Tween20, 0.005% antifoam, 2mg/ml acetylated BSA (Invitrogen Life Technologies) and 10 μg/ml R phycoerythrin streptavidin (Molecular Probes, Eugene, Oreg.)). After first staining, the probe array was washed for 10 cycles of 4 mixes/cycle with non-stringent wash buffer at 25° C. The probe array was then stained for 10 minutes at 25° C. in antibody solution (100 mM MES, IM Na⁺, 0.05% Tween20, 0.005% antifoam, 2 mg/ml acetylated BSA (Invitrogen Life Technologies), 100 μg/ml Goat IgG (SIGMA,St. Louis, Mo.) and 3 μg/ml biotinylated anti-streptavidin antibody(goat) (Vector Laboratories). Following the second stain, the probe array is stained again for an additional 10 minutes at 25° C. in SAPE solution. Finally, the probe array is washed for 15 cycles of 4 mixes/cycle with non-stringent wash buffer at 30° C. Arrays were scanned using an Affymetrix gene chip scanner (Affymetrix, Santa Clara, Calif.). The scanner contains a scanning confocal microscope and uses an argon ion laser for the excitation source and emission is detected by a photomultipler tube at 530 nm bandpass filter (fluorscein 0 or 560 longpass filter (phycoerythrin).

Data analysis was performed using GENECHIP 3.0 or 4.0 software with normalizing/scaling to internal controls. For each patient, two parameters were used to filter the data: 1) “Absolute Decision,” which indicates the presence (P) or absence (A) of RNA of a gene within a given RNA sample; 2) “Frequency,” which measures the number of copies of a given RNA within a RNA sample, and this value is expressed as Copies per million transcripts. If a gene was called “Absent,” its frequency was not used to calculate the average frequency of the gene. If a gene was called “Absent” for more than four patients in the Hu6800 data; more than two patients in the HgU95A data, or more than six normals, no average frequency was calculated. Genes that had average frequencies for normal volunteers only were tagged “Normal” while those that had average frequencies for patients only were tagged “Disease.” The fold change in gene expression was calculated by dividing the average gene frequency of the patients by that of the normals. Genes selected for analysis met the following criteria: 1) a fold change greater than 1.95 or less than −1.95 and 2) those genes tagged as either “Normal” or “Disease.”

The results are set forth in Tables 1 and 2, which are attached at the end of the written description as pages 1-66 and 1-5, respectively, and specifically incorporated by reference herein. The data in one of the columns indicates the average frequency (“Avg FC”) in the patients divided by the average frequency in non-diseased subjects. An increased expression in patients relative to normals is indicated by the absence of a sign in front of the “Avg FC RA/normal” number, whereas a decrease in expression in patients relative to subjects is indicated by the presence of a “−” sign in front of the number (negative values are listed at the end of the Tables). “#DIV/0!” indicates an infinite number, resulting from expression levels in normals that are undetectable. Accession numbers, Affymetrix identifiers (“qualifiers”) and gene name are depicted in the Tables. The sequence of most genes are available on GenBank. Genes whose Accession Number is characterized by “HT- . . . ” or “HG- . . . ” are available in the TIGR database on the internet. Genes that seemed of particular significance are highlighted or marked with a star.

Table 1 shows genes identified using the Hu6800 Affymetrix chip. Table 2 shows the genes of the Hu6800 chip that gave a positive signal and that encode a kinase or a phosphatase. Table 3 shows the genes that represent 1.95 fold or greater change in patients compared to normal, identified using the U95 chip sets. Table 3 is attached at the end of the written description as pages 1-36, and is specifically incorporated by reference herein. The Tables indicate the chromosomal localization of the genes.

Interestingly, numerous genes which are up-regulated in R.A. patients are located on human chromosome 6 in a region (6p21.3) that contains the genes of the major histocompatibility complex (MHC) and tumor necrosis factor (TNF), suggesting that these genes may be of importance in R.A. Other genes of interest are kinases and phosphatases. Yet other genes which are of interest are those that are up- or down-regulated by a factor of at least two and those in which the ratio of induction could not be determined since no detectable signal was obtained in the normal controls (genes indicated as ““#DIV/0!”). Other genes of interest are those that are highlighted or marked with a star in the Tables.

### Example 2

### Identification of Genes which are Up- or Down-Regulated in an Animal Model of Rheumatoid Arthritis

This example describes the identification of several genes which are up- or down-regulated in mice having collagen induced arthritis (CIA) relative to normal mice. Gene expression was measured in paws of mice; PBMCs and in synovium.

CIA is an accepted animal model for rheumatoid arthritis. The disease was induced as follows in mice. Male DBA/1 (Jackson Laboratories, Bar Harbor, Me.) mice were used for all experiments. Arthritis was induced with the use of either chicken collagen type II (Sigma, St.Louis, Mo.) or bovine collagen type II (Chondrex, Redmond, Wash.). Chicken collagen was dissolved in 0.01 M acetic acid and emulsified with an equal volume of Complete Freund's adjuvant (CFA; Difco Labs, Detroit, Mich.) containing 1 mg/ml Mycobacterium tuberculosis (strain H37RA). 200 μg of chicken collagen was intradermally injected in the base of the tail on day 0. On day 21, mice were injected intraperitoneally with a PBS solution containing 100 μg of chicken collagen II. Bovine collagen type II (Chondrex, Redmond, Wash.) was dissolved in 0.1 M acetic acid and emulsified in an equal volume of CFA (Sigma) containing 1 mg/ml Mycobacterium tuberculosis (strain H37RA). 200 μg of bovine collagen was injected subcutaneously in the base of the tail on day 0. On day 21, mice were injected subcutaneously, in the base of the tail, with a solution containing 200 μg of bovine collagen in 0.1 M acetic acid that had been mixed with an equal volume of Incomplete Freund's adjuvant (Sigma). Naive animals received the same sets of injections, minus collagen. Mice were monitored at least three times a week for disease progression. Individual limbs were assigned a clinical score based on the index: 0=normal; P=prearthritic, characterized by focal erythema on the tips of digits.; 1=visible erythema accompanied by 1-2 swollen digits.; 2=pronounced erythema, characterized by paw swelling and/or multi digit swelling.; 3=massive swelling extending into ankle or wrist joint.; 4=difficulty in use of limb or joint rigidity. The sum of all limb scores for any given mouse could yield a maximum total body score of 16.

At various stages of disease, animals were euthanized and tissues were harvested. In one series of examples, at least two paws from each animal were flash frozen in liquid nitrogen for RNA analyses. Frozen mouse paws were pulverized to a fine powder with the use of a mortar and pestle and liquid nitrogen. RNA was purified using the Promega RNAgents Total RNA Isolation System (Promega, Madison, Wis.). The RNA was further purified using the RNeasy minikit. The remaining paws were fixed in 10% formalin for histology.

In another series of examples, gene expression was determined in PBMCs of mice. Blood was collected via cardiac puncture into EDTA coated collection tubes. Blood samples were pooled according to similar total body scores (normal, prearthritic, scores 1, 3, 4, 5, 6, and 7-9) into a 15 ml conical tube. The blood was diluted 1:1 with PBS that contained 2 mM EDTA, and layered on an equal volume of Lympholyte-M (Cedar Lane Labs, Homby, Ontario, Canada). The mixture was centrifuged, with no brake, for 20 minutes at 1850 rpm in a Sorvall centrifuge, (model RT 6000D). Cells at the interface were collected and added to a new tube. The cells were washed with the addition of 10 ml PBS, containing 2 mM EDTA, and centrifuged at 1200 rpm for 10 minutes. The wash was repeated two times. To lyse residual red cells, cell pellets were dispersed in 2 ml of cold 0.2% NaCl and incubated on ice for 45-60 seconds. Lysis was terminated with the addition of 2 ml of 1.6% NaCl and the cells were centrifuged at 1200 rpm for 10 minutes. PBMCs were resuspended in 5 ml of PBS, which contained 2 mM EDTA, and counted. Cells were centrifuged at 1200 rpm for 10 minutes, and the supernatant discarded in preparation for RNA isolation. Total RNA was isolated from the PBMCs using the RNeasy minikit (Qiagen, Hidden, Germany).

In yet another series of examples, RNA was obtained from isolated synovium of the diseased animals. The joint synovium was dissected from diseased and control animals under a dissection scope. Tissues from five or more animals with similar disease scores were pooled and RNA was isolated using the RNeasy kit (Qiagen, Hidden, Germany).

Gene expression was analyzed on the oligonucleotide arrays Affymetrix murine 11K chip set composed of 11,000 murine genes on two chips, murine 11 KsubA P/N 900188 and murine llKsubB PIN900189.

Labeled target nucleic acids for hybridization to the chips were prepared as described in the previous Example with 5 μg of PBMC RNA or 7 μg of RNA from paws or synovial tissue.

Data analysis was performed using GENECHIP 3.0 software with normalizing/scaling to internal controls. Each experimental sample was compared to a time matched control in a two-file analysis. Next, the data were entered into the GeneSpring (Silicon Genetics, Redwood City, Calif.) analysis program. The data were filtered in a hierarchical fashion. First, the data were grouped according to paw scores. For each score, a list of genes that were called “Present” in all samples in a given score group and in the control was created. These lists were further refined by removing all genes that were not called either “Increasing” or “Decreasing” (defined in the program) in at least a majority of the samples in each score group. These lists were then filtered for genes that showed fold change greater than or equal to 1.95 or less than or equal to −1.95 in either all of the samples, if there were less than five samples, or in greater than 70% of the samples.

The results of the PBMCs are indicated in Table 4 and the results of the paw examples are indicated in Table 5. Tables 4 and 5 are attached at the end of the written descripion as pages 1-17 and 1-74, respectively, and are specifically incorporated by reference herein. “C” stands for control; “P” stands for prearthitic. The columns represent fold changes compared back to the normal. Accession numbers, Affymetrix identifiers (“qualifiers”) and gene name are depicted in the Tables.

The results show that several genes, e.g., PTPN18, HMG-1 and SLPI, that are significantly up-regulated in the mouse model, were also significantly up-regulated in human PBMCs of R.A. patients.

### Example 3

### Identification of Cells Expressing Genes which are Up-Regulated in R.A.

This Example describes the identity of cells expressing genes which are up-regulated in R.A. by in situ hybridization.

Paws of CIA mice were fixed in 4% paraformaldeyde, pH 7.47, decalcified in 20% EDTA (pH 8.0) and embedded in paraffin for in situ hybridization according to methods known in the art.

Sense and anti-sense riboprobes for use in the in situ hybridization were produced by generating 2 independent PCR products, as follows. T7 RNA polymerase binding sites were incorporated into the oligonucelotides to insert T7 binding sites at either the 5′end of the PCR product for sense riboprobe or the 3′end of the PCR product for antisense riboprobe. Digoxygenin labeled probes were prepared with the use of a DIG RNA labeling mix (Roche Diagnostics, Mannheim, Germany), as described by the manufacturer, and T7 RNA polymerase (Roche Diagnostics).

The probes were obtained by PCR using the following oligonucleotide primers for each of the sense and antisense probe.

Murine SAA3 Sense Riboprobe:

Forward primer (with T7 site):

| | |
|---|---|
| 5′GACTGATAATACGACTCACTATAGGGCGAATGAAG | (SEQ ID NO:1) |
| CCTTCCATTGCCATCATTCTTTGCA3′ | |

Reverse primer:

| | |
|---|---|
| 5′TTAGCGGCCGCTCAGTATCTTTTAGGCAGGCCAGC | (SEQ ID NO:2) |

AGGTCGGAA3′

The probe sequence covers the entire coding sequence, is 369 nucleotides long and has the following sequence:

atgaagcctt ccattgccat cattctttgc atcttgatcc tgggagttga cagccaaaga tgggtccagt tcatgaaaga agctggtcaa gggtctagag acatgtggcg agcctactct gacatgaaga aagctaactg gaaaaactca gacaaatact tccatgctcg ggggaactat gatgctgccc ggaggggtcc cgggggagcc tgggctgcta aagtcatcag cgatgccaga gaggctgttc agaagttcac gggacatgga gcagaggact caagagctga ccagtttgcc aatgagtggg gccggagtgg caaagacccc aaccacttcc gacctgctgg cctgcctaaa agatactga (SEQ ID NO: 3)

Murine SAA3 Anti-Sense Riboprobe:

Forward primer:

| | |
|---|---|
| 5′TTAGAATTCATGAAGCCTTCCATTGCCATCATTCT | (SEQ ID NO:4) |

TTGCA3′

Reverse primer (with T7 site):

| | |
|---|---|
| 5′GACTGATAATACGACTCACTATAGGGCGATCAGTA | (SEQ ID NO:5) |

TCTTTTAGGCAGGCCAGCAGGTCGGAA3′

The probe sequence covers the entire coding sequence, is 369 nucleotides long and has the following sequence:

tcagtatctt ttaggcaggc cagcaggtcg gaagtggttg gggtctttgc cactccggcc ccactcattg gcaaactggt cagctcttga gtcctctgct ccatgtcccg tgaacttctg aacagcctct ctggcatcgc tgatgacttt agcagcccag gctcccccgg gacccctccg ggcagcatca tagttccccc gagcatggaa gtatttgtct gagtttttcc agttagcttt cttcatgtca gagtaggctc gccacatgtc tctagaccct tgaccagctt ctttcatgaa ctggacccat ctttggctgt caactcccag gatcaagatg caaagaatga tggcaatgga aggcttcat (SEQ ID NO: 6)

Murine LST-1 Sense Riboprobe:

Forward primer (with T7 site):

| | |
|---|---|
| 5′GACTGATAATACGACTCACTATAGGGCGAATGTCT | (SEQ ID NO:7) |
| GATGACAATGGATCTGGTAACAATTGCA3′ | |

Reverse primer:

| | |
|---|---|
| 5′TTAGCGGCCGCTCAAGTGGGTGTGCTCCTGGCGAT | (SEQ ID NO:8) |

GCAGGCATA3′

The probe sequence covers the entire coding sequence, has 288 nucleotides and the following sequence:

atgtctgatg acaatggatc tggtaacaat tgcacaacca atcatttcct gctctatggg agcctgggac tgggagggct cctcctcctg cttgtcatca tcctgttcat ctgcctgtgc gggttcagtc agagagtgaa gagactggaa aggaatgccc aggtctcagg gcaggagccc cactatgcat ctctccagca gctgccagtg tccagtagtg atatcacaga catgaaagaa gacctcagca ctgactatgc ctgcatcgcc aggagcacac ccacttga (SEQ ID NO: 9)

Murine LST-1 Anti-Sense Riboprobe:

Forward primer:

| | |
|---|---|
| 5′TTAGAATTCATGTCTGATGACAATGGATCTGGTA | (SEQ ID NO:10) |

ACAATTGCA3′

Reverse primer (with T7 site):

| | |
|---|---|
| 5′GACTGATAATACGACTCACTATAGGGCGATC | (SEQ ID NO:11) |
| AAGTGGGTGTGCTCCTGGCGATGCAGGCATA3′ | |

The probe sequence covers the entire coding sequence, is 288nucleotides long and has the following sequence:

| | |
|---|---|
| tcaagtgggt gtgctcctgg cgatgcaggc | (SEQ ID NO:12) |
| atagtcagtg ctgaggtctt ctttcatgtc | |
| tgtgatatca ctactggaca ctggcagctg | |
| ctggagagat gcatagtggg gctcctgccc | |
| tgagacctgg gcattccttt ccagtctctt | |
| cactctctga ctgaacccgc acaggcagat | |
| gaacaggatg atgacaagca ggaggaggag | |
| ccctcccagt cccaggctcc catagagcag | |
| gaaatgattg gttgtgcaat tgttaccaga | |
| tccattgtca tcagacat | |

Murine FST1 Sense Riboprobe:

Forward primer (with T7 site):

| | |
|---|---|
| 5′GACTGATAATACGACTCACTATAGGGCGAATGTG | (SEQ ID NO:13) |
| GAAACGATGGCTGGCGCTCTCGCTG3′ | |

Reverse primer:

| | |
|---|---|
| 5′AGGAACAGACACAGCGATTGC3′ | (SEQ ID NO:14) |

The probe is 421 nucleotides long and has the following sequence:

atgtggaaac gatggctggc gctctcgctg gtgaccatcg ccctggtcca cggcgaggag gaacctagaa gcaaatccaa gatctgcgcc aatgtgtttt gtggagctgg cagggaatgt gccgtcacag agaaggggga gcccacgtgc ctctgcattg agcaatgcaa acctcacaag aggcctgtgt gtggcagtaa tggcaagacc tacctcaacc actgtgaact tcatagagat gcctgcctca ctggatccaa gatccaggtt gattatgatg ggcactgcaa agaaaagaag tctgcgagtc catctgccag cccagttgtc tgctatcaag ctaaccgcga tgagctccga cggcgcctca tccagtggct ggaagctgag atcattccag atggctggtt ctctaaaggc a (SEQ ID NO: 15)

Murine FST1 Anti-Sense Riboprobe:

Forward primer:

(SEQ ID NO:16)

5′GGGGATATCATGTGGAAACGATGGCTGGCGCTCTCGCTGGTGACCAT

3′

Reverse primer (with T7 site):

(SEQ ID NO:17)

5′GACTGATAATACGACTCACTATAGGGCGATGCCTTTAGAGAACCAGCC
ATCTGGAATGA3′

The probe is 421 nucleotides long and has the following sequence:

| | |
|---|---|
| tgcctttaga gaaccagcca tctggaatga tctcagcttc cagccactgg atgaggcgcc gtcggagctc atcgcggtta | (SEQ ID NO:18) |
| gcttgatagc agacaactgg gctggcagat ggactcgcag acttcttttc tttgcagtgc ccatcataat caacctggat | |
| cttggatcca gtgaggcagg catctctatg aagttcacag tggttgaggt aggtcttgcc attactgcca cacacaggcc | |
| tcttgtgagg tttgcattgc tcaatgcaga ggcacgtggg ctcccccttc tctgtgacgg cacattccct gccagctcca | |
| caaaacacat tggcgcagat cttggatttg cttctaggtt cctcctcgcc gtggaccagg gcgatggtca ccagcgagag | |
| cgccagccat cgtttccaca t | |

Murine SLPI Sense Riboprobe:

Forward primer (with T7 site):

| | |
|---|---|
| 5′GACTGATAATACGACTCACTATAGGGCGAATGAAGTCCTGCGGCCTTTTACCTTTC | (SEQ ID NO:18) |
| ACGGTG3′ | |

Reverse primer:

(SEQ ID NO:19)

5′AATGCGGCCGCTCACATCGGGGGCAGGCAGACTTTCCCAC3′

The probe sequence covers the entire coding sequences, is 396 nucleotides long and has the following sequence:

| | |
|---|---|
| atgaagtcct gcggcctttt acctttcacg gtgctccttg ctctggggat cctggcaccc tggactgtgg aaggaggcaa | (SEQ ID NO:20) |
| aaatgatgct atcaaaatcg gagcctgccc tgctaaaaag cctgcccagt gccttaagct tgagaagcca caatgccgta | |
| ctgactggga gtgcccggga aagcagaggt gctgccaaga tgcttgcggt tccaagtgcg tgaatcctgt tcccattcgc | |
| aaaccagtgt ggaggaagcc tgggaggtgc gtcaaaactc aggcaagatg tatgatgctt aaccctccca atgtctgcca | |
| gagggacggg cagtgtgacg gcaaatacaa gtgctgtgag ggtatatgtg ggaaagtctg cctgcccccg atgtga | |

Murine SLPI Anti-Sense Riboprobe:

Forward primer:

(SEQ ID NO:21)

5′ATTGAATTCATGAAGTCCTGCGGCCTTTTACCTTTCACGGTGC3′

Reverse primer (with T7 site):

| | |
|---|---|
| 5′GACTGATAATACGACTCACTATAGGGCGATCACATCGGGGGCAGGCAGACTTTCC | (SEQ ID NO:22) |
| CAC3′ | |

The probe sequence covers the entire coding equence, is 396 nucleotides long and has the following sequence:

| | |
|---|---|
| tcacatcggg ggcaggcaga ctttcccaca tataccctca cagcacttgt atttgccgtc acactgcccg tccctctggc | (SEQ ID NO:23) |
| agacattggg agggttaagc atcatacatc ttgcctgagt tttgacgcac ctcccaggct tcctccacac tggtttgcga | |
| atgggaacag gattcacgca cttggaaccg caagcatctt ggcagcacct ctgctttccc gggcactccc agtcagtacg | |
| gcattgtggc ttctcaagct taaggcactg ggcaggcttt ttagcagggc aggctccgat tttgatagca tcatttttgc | |
| ctccttccac agtccagggt gccaggatcc ccagagcaag gagcaccgtg aaaggtaaaa ggccgcagga cttcat | |

Murine Legumain Sense Riboprobe:

Forward primer (with T7 site):

| | |
|---|---|
| 5′GACTGATAATACGACTCACTATAGGGCGA ACACCAACACCAGCCATGTC3′ | (SEQ ID NO:24) |

Reverse primer:

| | |
|---|---|
| 5′CTCTCAGCAGTTTCCCCAAATC3′ | (SEQ ID NO:25) |

The probe is 313 nucleotides long and has the following sequence:

acaccaacac cagccatgtc atgcaatatg ggaacaaatc tatctctacc atgaaagtga tgcagtttca gggaatgaag cacagagcca gttcccccat ctccctgcct ccggtcacac accttgacct cacccccagc cctgacgtgc ccctgaccat cttgaagagg aagctgctga gaaccaacga cgtgaaggaa tcccagaatc tcattgggca gatccagcaa tttctggatg ccaggcacgt cattgagaag tctgtgcaca agatcgtttc cctgctggcg ggatttgggg aaactgctga gag (SEQ ID NO: 26)

Murine Legumain Anti-Sense Riboprobe:

Forward Primer:

| | |
|---|---|
| 5′ACACCAACACCAGCCATGTC3′ | (SEQ ID NO:27) |

Reverse primer (withT7 site):

| | |
|---|---|
| 5′GACTGATAATACGACTCACTATAGGGCGACTCTCAGCAGTTTCCCCAAATC3′ | (SEQ ID NO:28) |

The probe sequence is 313 nucleotides long and has the following sequence:

ctctcagcag tttccccaaa tcccgccagc agggaaacga tcftgtgcac agacttctca atgacgtgcc tggcatccag aaattgctgg atctgcccaa tgagattctg ggattccttc acgtcgttgg ttctcagcag cttcctcttc aagatggtca ggggcacgtc agggctgggg gtgaggtcaa ggtgtgtgac cggaggcagg gagatggggg aactggctct gtgcttcatt ccctgaaact gcatcacttt catggtagag atagatttgt tcccatattg catgacatgg ctggtgttgg tgt (SEQ ID NO: 29).

Sections of paraffin embedded tissue were de-paraffinized with xylene, 2 changes, 3 minutes each, and rehydrated to water. After a rinse in RNase-free water and phosphate buffered saline (PBS), permeabilization was performed by incubation with 0.2% Triton-X 100/PBS for 15 minutes. After 2 washes with PBS, each at 3 minutes, the sections were ready for proteinase K (PK)(Sigma) treatment. Sections were immersed in 0.1M Tris and 50 mM EDTA (Sigma) (pH 8.0) pre-warmed at 37° C. containing 5 μg/ml PK for 15 minutes. PK activities were stopped by 0.1M glysine/PBS for 5 minutes followed by a post fixation with 4% paraformaldehyde for 3 minutes and PBS rinsed. To prevent non-specific electrostatic binding of the probe, sections were immersed in 0.25% acetic anhydride and 0.1M triethanolamine solution (pH 8.0) for 10 minutes, followed by 15 seconds in 20% acetic acid at 4° C. After 3 changes in PBS, 5 minutes each, sections were dehydrated through 70%, 90% and 100% ethanol, each at 3 minutes. The sections were completely air dried before 40 μl of pre-hybridization buffer was applied, covered with Parafilm and incubated at 52° C. for 30 minutes to reduce non-specific binding. Parafilm was removed and 40 μl of hybridization buffer containing 5 ng/μl of digoxigenin-labeled probe was applied to each section, recovered with Parafilm and incubated overnight at 52° C.

Parafilm was carefully removed and sections were immersed into 2× saline sodium citrate (SSC) (Sigma)/0.1% lauryl sulphate (SDS) (Sigma) at room temperature, 4 changes, 5 minutes each. To ensure specific binding of the probe, sections were washed in a high stringency solution containing 0.1× SSC/0.1% SDS at 52° C., 2 changes, 10 minutes each. Endogenous peroxidase was quenched by immersion of sections in 3% H₂O₂, 15 minutes at room temperature followed by 3 washes in PBS, 2 minutes each. The labeled probe was detected with anti-digoxigenin antibody conjugated to peroxidase complex (Roche) diluted 1:50 in 2% normal sheep serum/0.1% Triton X-100. Labeled probe was developed with DAB (Vector Laboratories), washed in water, stained briefly with hematoxylin, dehydrate in graded alcohol and mounted in Permount mountant (Fisher Scientific) before microscopic examination.

The results indicate that no staining was observed in any of the paws treated with the sense probes (negative control). Expression of all of the genes described below was detected in joints of animals with collagen induced arthritis. No staining was seen in untreated animals.

More particularly, individual cells expressing the RNAs tested for were identified. FSTl mRNA positive cells were neutrophils, macrophages, fibroblasts, osteoblasts. No FST1 was found in bone tissue. SAA-3 mRNA positive cells were, neutrophils, macrophages, fibroblasts, superficial epidermis and chondrocytes. No staining with SAA-3 was seen in the articular cartilage. SLP-1 mRNA positive cells were osteoblasts, fibroblast and a focal area of chondrogenesis. The macrophages seemed to be positive (mild), endothelial cells appeared to be positive (mild) and neutrophils seemed to be negative for SLP-1 mRNA. Lymphocytes were difficult to identify in the SLP-1 hybridized sections. Legumain mRNA positive cells were seen in the epidermis. Osteoblasts and fibroblast had positive cytoplasmic staining with the Legumain antisense probe. The macrophages were positive (mild) and neutrophils appeared to be negative for Legumain mRNA. Lymphocytes were difficult to identify in the Legumain hybridized sections.

Equivalents

It will be apparent to those skilled in the art that the examples and embodiments described herein are by way of illustration and not of limitation, and that other examples may be used without departing from the spirit and scope of the present invention, as set forth in the claims.

TABLE 1

| | | | 6800 chip human RA PBMC | | | | | | | | | | | | |
|---|---|---|---|---|---|---|---|---|---|---|---|---|---|---|---|
| | | | | | Avg | Nor- | | | Avg | | | | | | |
| | | | Patients | | Freq | mals | | Freq | | | | | | | |
| | | GeneSpring | called | #“P” | RA | called | (Nor- | #“P” | Nor- | | Fold | | | | |
| name | qualifier | qualifier | “P” > 4 | (RA) | Patients | “P” > 6 | mal) | (RA) | mals | Ratio | Change | Symbol | Chromosome | Description | function |
| MR110000 | D64154_at | D64154 | fail | 4 | | PASS | 13 | 4 | 9.77 | Normal | Normal | | | Mr110,000 antigen | |
| RAC2 | M64595_at | M64595 | fail | 3 | | PASS | 13 | 3 | 19.85 | Normal | Normal | RAC2 | 22q12-q13.2 | ras-related C3 botulinum | ras-related C3 botulinum |
| | | | | | | | | | | | | | | toxin substrate 2 (rho | toxin substrate 2 (rho |
| | | | | | | | | | | | | | | family, small GTP | family, small GTP |
| | | | | | | | | | | | | | | binding protein Rac2) | binding protein Rac2) |
| J03263_s_at | J03263_s_at | J03263 | fail | 3 | | PASS | 13 | 3 | 9.23 | Normal | Normal | LAMP1 | | | membrane glycoprotein |
| TBXAS1 | M80647_at | M80647 | fail | 4 | | PASS | 12 | 4 | 17.42 | Normal | Normal | TBXAS1 | 7q34-q35 | thromboxane A synthase 1 | thromboxane A synthase 1 |
| | | | | | | | | | | | | | | (platelet, cytochrome P450, | (platelet,cytochrome P450, |
| | | | | | | | | | | | | | | subfamily V) | subfamily V) |
| ALDR1 | J04794_at | J04794 | fail | 4 | | PASS | 12 | 4 | 14.42 | Normal | Normal | ALDR1 | | | aldehyde reductase |
| | | | | | | | | | | | | | | (EC 1.1.1 2) | |
| HADHA | D16480_at | D16480 | fail | 2 | | PASS | 12 | 2 | 16.33 | Normal | Normal | HADHA | 2p23 | hydroxyacyl-Coenzyme A | hydroxyacyl-Coenzyme A |
| | | | | | | | | | | | | | | dehydrogenase/3-ketoacyl- | dehydrogenase/3-ketoacyl- |
| | | | | | | | | | | | | | | Coenzyme A thiolase/ | Coenzyme A thiolase/ |
| | | | | | | | | | | | | | | enoyl-Coenzyme A | enoyl-Coenzyme A |
| | | | | | | | | | | | | | | hydratase (trifunctional | hydratase (trifunctional |
| | | | | | | | | | | | | | | protein), alpha su | protein), alpha subunit |
| M13929_s_at | M13929_s_at | M13929 | fail | 1 | | PASS | 12 | 1 | 9.33 | Normal | Normal | MYC | | c-myc-P64 protein | ORF 114; putative |
| HLK1 | U40462_at | U40462 | fail | 1 | | PASS | 12 | 1 | 6.42 | Normal | Normal | hlK-1 | | Ikaros/LyF-1-homolog | similar to mouse LyF-1, |
| | | | | | | | | | | | | | | | encoded by GenBank |
| | | | | | | | | | | | | | | | Accession Number S74708; |
| | | | | | | | | | | | | | | | similar to mouse Ikaros |
| | | | | | | | | | | | | | | | DNA-binding protein, |
| | | | | | | | | | | | | | | | Swiss-Prot Accession |
| | | | | | | | | | | | | | | | Number Q03267 |
| MANA2 | D63998_at | D63998 | fail | 1 | | PASS | 12 | 1 | 5.25 | Normal | Normal | MANA2 | 5 | mannosidase, alpha type II | mannosidase, alpha type II |
| ITBA2 | X92896_at | X92896 | fail | 0 | | PASS | 12 | 0 | 6.42 | Normal | Normal | ITBA2 | | | |
| PPP1R2 | U68111_at | U68111 | fail | 0 | | PASS | 12 | 0 | 5.17 | Normal | Normal | PPP1R2 | | protein phosphatase | |
| | | | | | | | | | | | | | | inhibitor 2 | |
| LCP2 | U93049_at | U93049 | fail | 3 | | PASS | 11 | 3 | 11.82 | Normal | Normal | FYB | | FYN-binding protein | FYN-binding protein |
| | | | | | | | | | | | | | | (FYB-120/130) | (FYB-120/130) |
| PCNA | J05614_at | J05614 | fail | 3 | | PASS | 11 | 3 | 11.73 | Normal | Normal | | | | |
| POLR2B | L37127_at | L37127 | fail | 2 | | PASS | 11 | 2 | 13.27 | Normal | Normal | | | RNA polymerase II | |
| DG | HG1872-HT1 | HG1872-HT | fail | 2 | | PASS | 11 | 2 | 10.55 | Normal | Normal | | | | |
| MEL | X56741_at | X56741 | fail | 2 | | PASS | 11 | 2 | 7.82 | Normal | Normal | rab8 | | rab8 small GTP binding | |
| U09178_s_at | U09178_s_at | U09178 | fail | 2 | | PASS | 11 | 2 | 6.91 | Normal | Normal | DPYD | 1p22 | dihydropyrimidine | dihydropyrimidine |
| | | | | | | | | | | | | | | dehydrogenase | dehydrogenase |
| CALM | U45976_at | U45976 | fail | 2 | | PASS | 11 | 2 | 6.91 | Normal | Normal | CALM | | CALM | |
| TLE4 | M99439_at | M99439 | fail | 2 | | PASS | 11 | 2 | 5.73 | Normal | Normal | TLE4 | | transducin-like enhancer | transducin-like enhancer |
| | | | | | | | | | | | | | | protein | of split 4, homolog of |
| | | | | | | | | | | | | | | | Drosophila E (spl) |
| HSPA4 | L12723_at | L12723 | fail | 2 | | PASS | 11 | 2 | 5.45 | Normal | Normal | HSPA4 | 5q31.1-q31.2 | Heat shock 70 kD protein 4 | heat shock 70 kD protein 4 |
| NUCP40 | U86602_at | U86602 | fail | 2 | | PASS | 11 | 2 | 5.09 | Normal | Normal | | | nucleolar protein p40 | cell proliferation- |
| | | | | | | | | | | | | | | | associated protein |
| E_CIT987SK | U91327_at | U91327 | fail | 1 | | PASS | 11 | 1 | 5.82 | Normal | Normal | 99D8.1 | | T-complex protein 1, Beta | |
| | | | | | | | | | | | | | | | subunit (TCP-1-BETA) |
| FRAP | L34075_at | L34075 | fail | 1 | | PASS | 11 | 1 | 5.73 | Normal | Normal | FRAP1 | 1p36.2 | FKBP-rapamycin | FK506 binding protein 12- |
| | | | | | | | | | | | | | | associated protein | rapamycin associated |
| | | | | | | | | | | | | | | | protein 1 |
| EIF2G | L19161_at | L19161 | fail | 1 | | PASS | 11 | 1 | 5.64 | Normal | Normal | EIF2S3 | Xp22.2-p22.1 | eukaryotic translation | eukaryotic translation |
| | | | | | | | | | | | | | | initiation factor 2, subunit | initiation factor 2, subunit |
| | | | | | | | | | | | | | | 3, (gamma, 52 kD) | 3 (gamma, 52 kD) |
| P_E46 | Z93784_at | Z93784 | fail | 1 | | PASS | 11 | 1 | 5.36 | Normal | Normal | dJ398C22 1 | | dJ398C22.1 | E46-like contains exons |
| | | | | | | | | | | | | | | | 2-9 continues in Z84478 |
| UCHL3 | M30496_at | M30496 | fail | 1 | | PASS | 11 | 1 | 4.27 | Normal | Normal | | | | ubiquitin carboxyl-terminal |
| | | | | | | | | | | | | | | | hydrolase |
| RPA1 | M63488_at | M63488 | fail | 0 | | PASS | 11 | 0 | 7.45 | Normal | Normal | RPA1 | 17 | replication protein A1 | replication protein A1 |
| | | | | | | | | | | | | | | (70 kD) | (70 kD) |
| RIINF | HG511-HT51 | HG511-HT | fail | 0 | | PASS | 11 | 0 | 5.36 | Normal | Normal | | | | |
| M26041_s_at | M26040_s_at | M26041 | fail | 3 | | PASS | 10 | 3 | 20.70 | Normal | Normal | LA-DQA1 | 6p21.3 | major histocompatibility | major histocompatibility |
| | | | | | | | | | | | | | | complex, class II, | complex, class II, |
| | | | | | | | | | | | | | | DQ alpha 1 | DQ alpha 1 |
| K91_PCSK | D42053_at | D42053 | fail | 2 | | PASS | 10 | 2 | 6.70 | Normal | Normal | S1P | 16 | site-1 protease (subtilisin- | site-1 protease (subtilisin- |
| | | | | | | | | | | | | | | like, sterol-regulated, | like, sterol-regulated, |
| | | | | | | | | | | | | | | cleaves sterol regulatory | cleaves sterol regulatory |
| | | | | | | | | | | | | | | element binding proteins) | element binding proteins) |
| KCNQ1 | U40990_at | U40990 | fail | 2 | | PASS | 10 | 2 | 6.70 | Normal | Normal | KVLQT1 | | voltage gated potassium | |
| GALC | L23116_at | L23116 | fail | 2 | | PASS | 10 | 2 | 5.40 | Normal | Normal | GALC | 14q31 | galactosylceramidase | galactosylceramidase |
| | | | | | | | | | | | | | | (Krabbe disease) | Krabbe disease) |
| KPNB3 | U72761_at | U72761 | fail | 2 | | PASS | 10 | 2 | 5.40 | Normal | Normal | KPNB3 | | karopherin (importin) | karyopherin (importin) |
| | | | | | | | | | | | | | | beta 3 | beta 3 |
| DR1 | M97388_at | M97388 | fail | 2 | | PASS | 10 | 2 | 5.30 | Normal | Normal | DR1 | 1p22.1 | down-regulator of | down-regulator of |
| | | | | | | | | | | | | | | transcription 1, TBP- | transcription 1, TBP- |
| | | | | | | | | | | | | | | binding (negative | binding (negative |
| | | | | | | | | | | | | | | cofactor 2) | cofactor 2) |
| RFC4 | M87339_at | M87339 | fail | 2 | | PASS | 10 | 2 | 5.20 | Normal | Normal | RFC4 | 3127 | replication factor C | replication factor C |
| | | | | | | | | | | | | | | (activator 1) 4 (37 kD) | (activator 1) 4 (37 kD) |
| BIOBM | AFFX-BioB-1 | AFFX-BioI | fail | 1 | | PASS | 10 | 1 | 7.20 | Normal | Normal | | | | |
| UBE2D1 | HG3344-HT3 | HG3344-HT | fail | 1 | | PASS | 10 | 1 | 6.50 | Normal | Normal | | | | |
| MANA2 | L28821_at | L28821 | fail | 1 | | PASS | 10 | 1 | 6.30 | Normal | Normal | MAN2A2 | 15q25 | alpha mannosidase II | mannosidase, alpha, class |
| | | | | | | | | | | | | | | isozyme | 2A, member 2 |
| CAMKA2 | U81554 at | U81554 | fail | 1 | | PASS | 10 | 1 | 5.50 | Normal | Normal | CAMK2G | 10q22 | calcium/calmodulin- | calcium/calmodulin- |
| | | | | | | | | | | | | | | dependent protein kinase | dependent protein kinase |
| | | | | | | | | | | | | | | (CaM kinase) II gamma | (CaM kinase) II gamma |
| HG2797-HT2 | HG2797-HT2 | HG2797-HT | fail | 1 | | PASS | 10 | 1 | 5.20 | Normal | Normal | | | | |
| POH1 | U86782_at | U86782 | fail | 1 | | PASS | 10 | 1 | 5.00 | Normal | Normal | POH1 | | 26S proteasome-associated | human homolog of fission |
| | | | | | | | | | | | | | | pad 1 homolog | yeast pad1 |
| GZMM | HG3104-HT3 | HG3104-HT | fail | 0 | | PASS | 10 | 0 | 16.20 | Normal | Normal | | | | |
| BAP | U72512_at | U72512 | fail | 3 | | PASS | 9 | 3 | 13.56 | Normal | Normal | | | | |
| ESD | D28416_at | D28416 | fail | 3 | | PASS | 9 | 3 | 10.89 | Normal | Normal | | | esterase D | |
| K01160_s_at | K01160_s_at | K01160 | fail | 3 | | PASS | 9 | 3 | 10.00 | Normal | Normal | | | | |
| U45878_s_at | U45878_s_at | U45878 | fail | 3 | | PASS | 9 | 3 | 9.22 | Normal | Normal | | | inhibitor of apoptosis | HIAP-1 |
| | | | | | | | | | | | | | | protein 1 | |
| RPS4Y | M58459_at | M58459 | fail | 2 | | PASS | 9 | 2 | 44.67 | Normal | Normal | RPS4Y | Yp11.3 | ribosomal protein S4, | ribosomal protein S4, |
| | | | | | | | | | | | | | | Y-linked | Y-linked |
| LTR | M92449_at | M92449 | fail | 2 | | PASS | 9 | 2 | 9.89 | Normal | Normal | PLT | | | putative |
| FBP1 | U05040_at | U05040 | fail | 2 | | PASS | 9 | 2 | 7.67 | Normal | Normal | FUBP | | FUSE-binding protein | far upstream element |
| | | | | | | | | | | | | | | | binding protein |
| CD27 | M63928_at | M63928 | fail | 2 | | PASS | 9 | 2 | 7.44 | Normal | Normal | TNFRSF7 | 12p13 | CD27 antigen | tumor necrosis factor |
| | | | | | | | | | | | | | | | receptor superfamily, |
| | | | | | | | | | | | | | | | member 7 |
| FGFR1 | U28811_at | U28811 | fail | 2 | | PASS | 9 | 2 | 6.11 | Normal | Normal | CFR-1 | | cysteine-rich fibroblast | |
| | | | | | | | | | | | | | | growth factor receptor | |
| PTPRA | M34668_at | M34668 | fail | 2 | | PASS | 9 | 2 | 5.67 | Normal | Normal | PTPRA | 20p13 | protein tyrosine phos- | protein tyrosine phos- |
| | | | | | | | | | | | | | | phatase, receptor type, | phatase, receptor type, |
| | | | | | | | | | | | | | | alpha polypeptide | alpha polypeptide |
| U52191_s_at | U52191_s_at | U52191 | fail | 2 | | PASS | 9 | 2 | 5.56 | Normal | Normal | SMCY | Yq | SMC (mouse) homolog, Y | SMC (mouse) homolog, Y |
| | | | | | | | | | | | | | | chromosome | chromosome |
| CBR | J04056_at | J04056 | fail | 2 | | PASS | 9 | 2 | 4.89 | Normal | Normal | CBR1 | 21q22.1 | carbonyl reductase 1 | carbonyl reductase 1 |
| HSPB1 | ZZ3090_at | Z23090 | fail | 1 | | PASS | 9 | 1 | 12.56 | Normal | Normal | HSPB1 | 7q | heat shock 27 kD protein 1 | heat shock 27 kD protein 1 |
| STAT1Mb | AFFX-HUMI | AFFX-HUM | fail | 1 | | PASS | 9 | 1 | 7.00 | Normal | Normal | | | | |
| K129_RFPTE | D50919_at | D50519 | fail | 1 | | PASS | 9 | 1 | 4.89 | Normal | Normal | KIAA0129 | | | KIAA0129 gene product |
| CDK7 | L20320_at | L20320 | fail | 1 | | PASS | 9 | 1 | 4.89 | Normal | Normal | CDK7 | 2p15-cen | cyclin-dependent kinase 7 | cyclin-dependent kinase 7 |
| | | | | | | | | | | | | | | (homolog of Xenopus | (homolog of Xenopus |
| | | | | | | | | | | | | | | MO15 cdk-activating | MO15 cdk-activating |
| | | | | | | | | | | | | | | kinase) | kinase) |
| FABP5 | M94856_at | M94856 | fail | 1 | | PASS | 9 | 1 | 4.78 | Normal | Normal | FABP5 | | fatty acid binding | fatty acid binding |
| | | | | | | | | | | | | | | protein 5 (psoriasis- | protein 5 (psoriasis- |
| | | | | | | | | | | | | | | associated) | associated) |
| ICSBP1 | M91196_at | M91196 | fail | 0 | | PASS | 9 | 0 | 8.33 | Normal | Normal | ICSBP1 | | interferon consensus | interferon consensus |
| | | | | | | | | | | | | | | sequence binding | sequence binding |
| | | | | | | | | | | | | | | protein 1 | protein 1 |
| NMT1 | M86707_at | M86707 | fail | 0 | | PASS | 9 | 0 | 7.33 | Normal | Normal | NMT1 | | | N-myristoyltransferase 1 |
| RAB4 | M28211_at | M28211 | fail | 0 | | PASS | 9 | 0 | 5.11 | Normal | Normal | RAB4 | 1q42-q43 | RAB4, member RAS | RAB4, member RAS |
| | | | | | | | | | | | | | | oncogene family | oncogene family |
| ERPRT | M27826_at | M27826 | fail | 2 | | PASS | 8 | 2 | 10.63 | Normal | Normal | | | neutral protease large | Xxx; putative |
| | | | | | | | | | | | | | | subunit | |
| EV12A | M55267_at | M55267 | fail | 2 | | PASS | 8 | 2 | 10.13 | Normal | Normal | EVI2A | | EVI2 protein | |
| H2BH_f | Z80780_f_at | Z80780 | fail | 1 | | PASS | 8 | 1 | 9.88 | Normal | Normal | H2B/h | | histone H2B | |
| TIP60 | U74667_at | U74667 | fail | 0 | | PASS | 8 | 0 | 7.25 | Normal | Normal | TIP60 | | tat interactive protein | interacts with HIV1 Tat, |
| | | | | | | | | | | | | | | | similar to yeast SAS2, |
| | | | | | | | | | | | | | | | SAS3 and human MOZ, |
| | | | | | | | | | | | | | | | encoded by GenBank |
| | | | | | | | | | | | | | | | Accession Numbers |
| | | | | | | | | | | | | | | | U14548, Z23261 and |
| | | | | | | | | | | | | | | | U47742, respectively; |
| | | | | | | | | | | | | | | | similar to sequence with |
| | | | | | | | | | | | | | | | GenBank Accession |
| | | | | | | | | | | | | | | | Number U40989 |
| PHB | S85655_at | S85655 | fail | 0 | | PASS | 8 | 0 | 6.63 | Normal | Normal | PHB | 17q21 | prohibition | prohibition |
| EPHB4 | U07695_at | U07695 | fail | 0 | | PASS | 8 | 0 | 6.50 | Normal | Normal | EPHB4 | 7 | EphB4 | EphB4 |
| SNAP23 | U55936_at | U55936 | fail | 0 | | PASS | 8 | 0 | 6.00 | Normal | Normal | SNAP23 | | synaptosomal-associated | synaptosomal-associated |
| | | | | | | | | | | | | | | protein, 23 kD | protein, 23 kD |
| D26155_s_at | D26155_s_at | D26155 | fail | 0 | | PASS | 8 | 0 | 5.13 | Normal | Normal | SMARCA2 | 9p24-p23 | SWI/SNF related, matrix | SWI/SNF related, matrix |
| | | | | | | | | | | | | | | associated, actin dependent | associated, actin dependent |
| | | | | | | | | | | | | | | regulator of chromatin, | regulator of chromatin, |
| | | | | | | | | | | | | | | subfamily a, member 2 | subfamily a, member 2 |
| PRP4H | U48736_at | U48736 | fail | 0 | | PASS | 8 | 0 | 5.00 | Normal | Normal | PRP4 | | serine/threonine-protein | serine/threonine-protein |
| | | | | | | | | | | | | | | kinase PRP4 homolog | kinase PRP4 homolog |
| IL16 | HG270-HT27 | HG270-HT | fail | 0 | | PASS | 8 | 0 | 4.75 | Normal | Normal | | | | |
| E_E18CPGE | HG3991-HT4 | HG3991-HT | fail | 4 | | PASS | 7 | 4 | 30.57 | Normal | Normal | | | | |
| RORET | U90547_at | U90547 | fail | 4 | | PASS | 7 | 4 | 12.14 | Normal | Normal | RoRet | | Ro/SSA ribonucleoprotein | |
| | | | | | | | | | | | | | | homolog | |
| HMGIY_rna1 | LI7131_rna1 | L17131 | fail | 4 | | PASS | 7 | 4 | 9.71 | Normal | Normal | HMGIY | 6p | high-mobility group | high-mobility group |
| | | | | | | | | | | | | | | (nonhistone chromosomal) | (nonhistone chromosomal) |
| | | | | | | | | | | | | | | protein isoforms I and Y | protein isoforms I and Y |
| AFFX-BioDn | AFFX-BioDn | AFFX-BioI | fail | 2 | | PASS | 7 | 2 | 12.29 | Normal | Normal | | | | |
| TXBP181 | U33822_at | U33822 | fail | 1 | | PASS | 7 | 1 | 9.86 | Normal | Normal | MAD1L1 | 7p22 | MAD1 (mitotic arrest | MAD1 (mitotic arrest |
| | | | | | | | | | | | | | | deficient, yeast, homolog)- | deficient, yeast, homolog)- |
| | | | | | | | | | | | | | | like 1 | like 1 |
| NUCB | U31342_at | U31342 | fail | 0 | | PASS | 7 | 0 | 6.14 | Normal | Normal | | | nucleobinding | |
| DPH2L | U34880_at | U34880 | fail | 0 | | PASS | 7 | 0 | 6.00 | Normal | Normal | DPH2L1 | 17p13.3 | diptheria toxin resistance | diptheria toxin resistance |
| | | | | | | | | | | | | | | protein required for | protein required for |
| | | | | | | | | | | | | | | diphthamide biosynthesis | diphthamide biosynthesis |
| | | | | | | | | | | | | | | (Saccharomyces)-like 1 | (Saccharomyces)-like 1 |
| TRAP1 | U12595_at | U12595 | fail | 0 | | PASS | 7 | 0 | 5.71 | Normal | Normal | TRAP1 | | tumor necrosis factor | TNF type 1 receptor |
| | | | | | | | | | | | | | | receptor associated protein | associated protein |
| X60003_s_at | X60003_s_at | X60003 | fail | 0 | | PASS | 7 | 0 | 5.43 | Normal | Normal | delta CREB | | | |
| 2OGCP_rna1 | X66114_rna1 | X66114 | fail | 0 | | PASS | 7 | 0 | 5.43 | Normal | Normal | SLC20A4 | 17p13.3 | solute carrier family 20 | solute carrier family 20 |
| | | | | | | | | | | | | | | (oxoglutarate carrier), | (oxoglutarate carrier), |
| | | | | | | | | | | | | | | member | member 4 |
| K196 | D83780_at | D83780 | fail | 0 | | PASS | 7 | 0 | 5.14 | Normal | Normal | KIAA0196 | | | KIAA0196 gene product |
| K52_SK12P | D29641_at | D29641 | fail | 0 | | PASS | 7 | 0 | 5.00 | Normal | Normal | KIAA0052 | | | |
| CUL4A | U58090_at | U58090 | fail | 0 | | PASS | 7 | 0 | 5.00 | Normal | Normal | CUL4A | | Hs-CUL-4A | cullin 4A |
| E_23707 | U79270_at | U79270 | fail | 0 | | PASS | 7 | ) | 5.00 | Normal | Normal | COX11 | 17q22 | cytochrome c oxidase | cytochrome c oxidase |
| | | | | | | | | | | | | | | subunit 11 | subunit 11 |
| BLK | S76617_at | S76617 | fail | 0 | | PASS | 7 | 0 | 4.71 | Normal | Normal | BLK | 8p23-p22 | B lymphoid tyrosine kinase | B lymphoid tyrosine kinase |
| U69140_s_at | U69140_s_at | U69140 | fail | 0 | | PASS | 7 | 0 | 4.71 | Normal | Normal | | | zyginII | synaptotagmin interacting |
| | | | | | | | | | | | | | | | protein; Human ortholog |
| | | | | | | | | | | | | | | | of rt qyginII |
| ERPL1 | X89211_at | X89211 | fail | 0 | | PASS | 7 | 0 | 4.71 | Normal | Normal | HERV-L | | | Human Endogenous |
| | | | | | | | | | | | | | | | Retrovirus-Like elements |
| | | | | | | | | | | | | | | | (HERV-L)/pseudo |
| PRTK1 | S76965_at | S76965 | fail | 0 | | PASS | 7 | 0 | 4.71 | Normal | Normal | protein | | protein kinase inhibitor | This sequence comes |
| | | | | | | | | | | | | kinase in- | | | from FIG. 1B, PKI |
| | | | | | | | | | | | | hibitor, PK1 | | | |
| X93511_s_at | X93511_s_at | X93511 | fail | 0 | | PASS | 7 | 0 | 4.00 | Normal | Normal | orf1 | | telomeric DNA binding | |
| | | | | | | | | | | | | | | protein | |
| MAGEP15 | U19796_at | U19796 | PASS | 7 | 23.29 | fail | 3 | 7 | | Disease | Disease | | | melanoma antigen p15 | |
| HG3148-HT3 | HG3148-HT3 | HG3148-II | PASS | 7 | 12.86 | fail | 3 | 7 | | Disease | Disease | | | | |
| MTA1 | U35113_at | U35113 | PASS | 7 | 6.71 | fail | 1 | 7 | | Disease | Disease | MTA1 | | metastasis associated 1 | metastasis associated 1 |
| HG4120-HT4 | HG4120-HT4 | HG4120-HT | PASS | 6 | 5.17 | fail | 3 | 6 | | Disease | Disease | | | | |
| AVPR1B | L37112_at | L37112 | PASS | 6 | 15.00 | fail | 3 | 6 | | Disease | Disease | | | vasopressin V3 receptor | |
| ACRV1_rna1 | S65583_rna1 | S65583 | PASS | 6 | 12.83 | fail | 3 | 6 | | Disease | Disease | SP-10 | | SP-10 | intra-acrosomal protein; |
| | | | | | | | | | | | | | | | This sequence comes from |
| | | | | | | | | | | | | | | | FIG. 3. Protein sequence |
| | | | | | | | | | | | | | | | is in conflict with the |
| | | | | | | | | | | | | | | | conceptual translation; |
| | | | | | | | | | | | | | | | mismatch(126[G-&gt; R]) |
| U57623_s_at | U57623_s_at | U57623 | PASS | 6 | 6.33 | fail | 3 | 6 | | Disease | Disease | FABP3 | 1p33-p32 | fatty acid binding protein 3, | fatty acid binding protein 3, |
| | | | | | | | | | | | | | | muscle and heart | muscle and heart |
| | | | | | | | | | | | | | | (mammary-derived | (mammary-derived |
| | | | | | | | | | | | | | | growth inhibitor) | growth inhibitor) |
| AACT_rna1 | X68733_rna1 | X68733 | PASS | 6 | 10.50 | fail | 3 | 6 | | Disease | Disease | ACT | | alpha1-antichymotrypsin | Protein sequence is in |
| | | | | | | | | | | | | | | | conflict with the con- |
| | | | | | | | | | | | | | | | ceptual translation. |
| D29675_s_at | D29675_s_at | D29675 | PASS | 6 | 16.50 | fail | 2 | 6 | | Disease | Disease | | | | |
| SLO | U02632_at | U02632 | PASS | 6 | 5.67 | fail | 2 | 6 | | Disease | Disease | KCNMA1 | 10 | portassium large | potassium large |
| | | | | | | | | | | | | | | conductance calcium- | conductance calcium- |
| | | | | | | | | | | | | | | channel, subfamily M, | channel, subfamily M, |
| | | | | | | | | | | | | | | alpha member 1 | alpha member 1 |
| MME | J03779_at | J03779 | PASS | 6 | 28.50 | fail | 1 | 6 | | Disease | Disease | MME | 3q21-q27 | membrane metallo- | membrane metall- |
| | | | | | | | | | | | | | | endopeptidase (neutral | endopeptidase (Neutral |
| | | | | | | | | | | | | | | endopeptidase, | endopeptidase, |
| | | | | | | | | | | | | | | enkephalinase, | enkephalinase, |
| | | | | | | | | | | | | | | CALLA, CD10) | CALLA, CD10) |
| K246_NOTC | D87433_at | D87433 | PASS | 6 | 38.33 | fail | 1 | 6 | | Disease | Disease | KIAA0246 | | | |
| MDC | U83171_at | U83171 | PASS | 6 | 14.00 | fail | 0 | 6 | | Disease | Disease | SCYA22 | 16q13 | small inducible cytokine | small inducible cytokine |
| | | | | | | | | | | | | | | subfamily A ()Cys—Cys), | subfamily A (Cys—Cys), |
| | | | | | | | | | | | | | | member 22 | member 22 |
| M22403_s_at | M22403_s_at | M22403 | PASS | 5 | 6.40 | fail | 1 | 5 | | Disease | Disease | GP1BA | 17pter-p12 | glycoprotein Ib (platelet), | glycoprotein Ib (platelet), |
| | | | | | | | | | | | | | | alpha polypeptide | alpha polypeptide |
| FSTRP | U06863_at | U06863 | PASS | 5 | 8.80 | fail | 1 | 5 | | Disease | Disease | | | follistatin-related protein | |
| | | | | | | | | | | | | | | precursor | |
| PLCG2H | U45974_at | U45974 | PASS | 5 | 15.40 | fail | 0 | 5 | | Disease | Disease | | | | phosphatidylinositol (4,5) |
| | | | | | | | | | | | | | | | bisphosphate 5-phosphatase |
| | | | | | | | | | | | | | | | homolog |
| PTPRN | L18983_at | L18983 | PASS | 5 | 20.00 | fail | 0 | 5 | | Disease | Disease | PTPRN | 2q35-q36.1 | protein tyrosine phos- | protein tyrosine phos- |
| | | | | | | | | | | | | | | phatase, receptor type, N | phatase, receptor type, N |
| AQP9 | AB006190_at | AB006190 | PASS | 5 | 10.20 | fail | 0 | 5 | | Disease | Disease | AQP7 | 9p13 | aquasporin 7 | aquasporin 7 |
| EFNB3 | U66406_at | U66406 | PASS | 5 | 7.40 | fail | 0 | 5 | | Disease | Disease | EFNB3 | 17p13.1-p11.2 | ephrin-B3 | ephrin-B3 |
| M87789_s_at | M87789_s_at | | | | | | | | | | | | | | |
| M87789_s_at | M87789_s_at | M87789 | PASS | 8 | 118.00 | PASS | 9 | 8 | 19.56 | 6.03 | 6.03 | | | IgG | Anti-hepatitis A; putative |
| OC116 | U45285_at | U45285 | PASS | 9 | 31.44 | PASS | 10 | 9 | 7.30 | 4.31 | 4.31 | OC-116 kDa | | specific 116 kDa vacuolar | ATPase, H+transporting |
| | | | | | | | | | | | | | | proton pump subunit | |
| TETTRL | L11669_at | L11669 | PASS | 6 | 28.33 | PASS | 11 | 6 | 7.18 | 3.95 | 3.95 | ADD1 | 4p16.3 | adducin 1 (alpha) | adducin 1 (alpha) |
| CSF3R | M59820_at | M59820 | PASS | 6 | 43.67 | PASS | 7 | 6 | 11.57 | 3.77 | 3.77 | CSF3R | 1p35-p34.3 | colony stimulating factor 3 | colony stimulating factor 3 |
| | | | | | | | | | | | | | | receptor (granulocyte) | receptor (granulocyte) |
| IGF2 | S73149 at | S73149 | PASS | 8 | 35.13 | PASS | 9 | 8 | 9.33 | 3.76 | 3.76 | orf in intron | | | |
| | | | | | | | | | | | | 7 of insulin- | | | |
| | | | | | | | | | | | | like growth | | | |
| | | | | | | | | | | | | factor II | | | |
| | | | | | | | | | | | | gene | | | |
| 18SRNAM | AFFX-HUMI | AFFX-HUM | PASS | 6 | 28.67 | PASS | 7 | 6 | 7.71 | 3.72 | 3.72 | | | | |
| 18SRNA3 | AFFX-HUMI | AFFX-HUM | PASS | 9 | 46.89 | PASS | 11 | 9 | 12.64 | 3.71 | 3.71 | | | | |
| PROTEIN_II | V01512_ma1 | V01512 | PASS | 9 | 51.44 | PASS | 13 | 9 | 13.92 | 3.69 | 3.69 | FOS | 14q24.3 | v-fos FBJ murine | |
| | | | | | | | | | | | | | | | osteosarcoma viral |
| | | | | | | | | | | | | | | | oncogene homolog |
| ETR101 | M62831_at | M62831 | PASS | 9 | 105.67 | PASS | 13 | 9 | 28.69 | 3.68 | 3.68 | ETR101 | 19 | immediate early protein | immediate early protein |
| DIA1 | M28713_at | M28713 | PASS | 9 | 33.33 | PASS | 12 | 9 | 9.08 | 3.67 | 3.67 | DIA1 | 22q13.31-qter | cytochrome b5 reductase | diaphorase (NADH) |
| | | | | | | | | | | | | | | | (cytochrome b-5 reductase) |
| MX1 | M33882_at | M33882 | PASS | 7 | 34.71 | PASS | 9 | 7 | 9.56 | 3.63 | 3.63 | MX1 | 21q22.3 | myxovirus (influenza) | myxovirus (influenza) |
| | | | | | | | | | | | | | | resistance I, homolog of | resistance I, homolog of |
| | | | | | | | | | | | | | | murine (interferon- | murine (interferon- |
| | | | | | | | | | | | | | | inducible protein p78) | inducible protein p78) |
| SELPLG | U25956_at | U25956 | PASS | 9 | 75.89 | PASS | 13 | 9 | 21.08 | 3.60 | 3.60 | SELPLG | 12q24 | selectin P ligand | selectin P ligand |
| LFP40 | U72206_at | U72206 | PASS | 5 | 33.40 | PASS | 10 | 5 | 9.30 | 3.59 | 3.59 | LFP40 | chr. 1 | guanine nucleotide | guanine nucleotide |
| | | | | | | | | | | | | | | regulatory factor | regulatory factor |
| BB1 | S82470_at | S82470 | PASS | 6 | 32.00 | PASS | 12 | 6 | 8.92 | 3.59 | 3.59 | BBI | | malignant cell expression- | |
| | | | | | | | | | | | | | | | enhanced gene/tumor |
| | | | | | | | | | | | | | | | progression-enhanced gene; |
| | | | | | | | | | | | | | | | This sequence comes from |
| | | | | | | | | | | | | | | | FIG. 4A |
| LYSPHAD | U56417_at | U56417 | PASS | 9 | 26.33 | PASS | 11 | 9 | 7.36 | 3.58 | 3.58 | | | lysophosphatidic acid | LPAAT-a; 1-acyl-sn- |
| | | | | | | | | | | | | | | acyltransferase-alpha | glycerol-3-phosphate acyl- |
| | | | | | | | | | | | | | | | transferase; similar to |
| | | | | | | | | | | | | | | | sequence within class III |
| | | | | | | | | | | | | | | | MHC locus on chromosome |
| | | | | | | | | | | | | | | | 6 deposited in GenBank |
| | | | | | | | | | | | | | | | Accession Number U89336 |
| HG4535-HT4 | HG4535-HT4 | HG4535-HT | PASS | 8 | 3850 | PASS | 8 | 8 | 10.88 | 3.54 | 3.54 | | | | |
| ZYX | X95735_at | X95735 | PASS | 8 | 41.38 | PASS | 10 | 8 | 11.70 | 3.54 | 3.54 | ZYX | 7132 | zyxin | zyxin |
| S71043_rna1 | S71043_rna1 | S71043 | PASS | 9 | 88.33 | PASS | 13 | 9 | 25.92 | 3.41 | 3.41 | Ig & lt; | | immunoglobulin A heavy | This sequence comes from |
| | | | | | | | | | | | | alpha & gt; 2 | | chain allotype 2 | FIG. 3; IgA2 H chain |
| HD | L12392_at | L12392 | PASS | 9 | 22.33 | PASS | 9 | 9 | 6.56 | 3.41 | 3.41 | HD | 4p16.3 | huntingtin | huntingtin (Huntington |
| | | | | | | | | | | | | | | | disease) |
| ILK | U40282_at | U40282 | PASS | 9 | 29.22 | PASS | 12 | 9 | 8.58 | 3.40 | 3.40 | ILK | 11p15.5-p15.4 | integrin-linked kinase | integrin-linked kinase |
| PKM2 | X56494_at | X56494 | PASS | 8 | 63.50 | PASS | 13 | 8 | 18.77 | 3.38 | 3.38 | PKM2 | 15q22-qter | pyruvate kinase, muscle | pyruvate kinase, muscle |
| CD63_rna1 | X62654_rna1 | X62654 | PASS | 9 | 41.78 | PASS | 13 | 9 | 12.38 | 3.37 | 3.37 | CD63 | 12q12-q13 | CD63 antigen (melanoma | CD63 antigen (melanoma 1 |
| | | | | | | | | | | | | | | antigen) | antigen) |
| SA | M60922_at | M60922 | PASS | 8 | 55.88 | PASS | 12 | 8 | 16.58 | 3.37 | 3.37 | FLOT2 | 17g11-q12 | flotillin 2 | flotillin 2 |
| X62083_s_at | X62083_s_at | X62083 | PASS | 9 | 70.89 | PASS | 13 | 9 | 21.15 | 3.35 | 3.35 | FSH | | | |
| J03260_s_at | J03260_s_at | J03260 | PASS | 7 | 28.71 | PASS | 7 | 7 | 8.57 | 3.35 | 3.35 | GNAZ | 22q11.1-q11.2 | guanine nucleotide | guanine nucleotide |
| | | | | | | | | | | | | | | binding protein (G | binding protein (G |
| | | | | | | | | | | | | | | protein), alpha 2 | protein), alpha 2 |
| | | | | | | | | | | | | | | polypeptide | polypeptide |
| CDC25 | S78187_at | S78187 | PASS | 9 | 63.89 | PASS | 13 | 9 | 19.08 | 3.35 | 3.35 | CDC25B | 20p13 | cell division cycle | cell division cycle |
| | | | | | | | | | | | | | | 25B | 25B |
| RELA | L19067_at | L19067 | PASS | 9 | 39.78 | PASS | 10 | 9 | 11.90 | 3.34 | 3.34 | | | NF-kappa-B transcrip- | putative |
| | | | | | | | | | | | | | | tion factor subunit | |
| XQTP | D16469_at | D16469 | PASS | 9 | 31.67 | PASS | 11 | 9 | 9.55 | 3.32 | 3.32 | ATP6S1 | Xq28 | ATPasc, H+ transporting, | ATPase, H+ transporting, |
| | | | | | | | | | | | | | | lysosomal (vacuolar proton | lysosomal (vacuolar proton |
| | | | | | | | | | | | | | | pump), subunit 1 | pump), subunit 1 |
| RAGE_cds1 | U89336_cds1 | U89336 | PASS | 9 | 71.78 | PASS | 13 | 9 | 21.69 | 3.31 | 3.31 | HBX2 | | homeobox PBX2 gene | intron-exon boundaries |
| | | | | | | | | | | | | | | | identified by a contig of |
| | | | | | | | | | | | | | | | ESTs with GenBank |
| | | | | | | | | | | | | | | | Accession Numbers |
| | | | | | | | | | | | | | | | W76064, R59617, W72507 |
| K154_ADTG | D63876_at | D63876 | PASS | 9 | 33.89 | PASS | 12 | 9 | 10.25 | 3.31 | 3.31 | KIAA0154 | | | KIAA0154 gene product |
| | | | | | | | | | | | | | | | is related to mouse |
| | | | | | | | | | | | | | | | gamma adaptin. |
| PRSM1 | U58048_at | U58048 | PASS | 8 | 18.63 | PASS | 9 | 8 | 5.67 | 3.29 | 3.29 | PRSM1 | 16q24.3 | protease, metallo, 1, 33 kD | protease, metallo, 1, 33 kD |
| ATP6C | M62762_at | M62762 | PASS | 9 | 69.67 | PASS | 13 | 9 | 21.23 | 3.28 | 3.28 | ATP6C | 16p13.3 | ATPasc, H+ transporting | ATPase, H+ transporting, |
| | | | | | | | | | | | | | | lysosomal (vacuolar proton | lysosomal (vacuolar proton |
| | | | | | | | | | | | | | | pump) 16 kD | pump) 16 kD |
| NCF1 | M55067_at | M55067 | PASS | 9 | 72.33 | PASS | 13 | 9 | 22.09 | 3.28 | 3.28 | NCF1 | 7q11.23 | neutrophil cytosolic factor 1 | neutrophil cytosolic factor 1 |
| | | | | | | | | | | | | | | | (47 kD, chronic |
| | | | | | | | | | | | | | | | granulomatous disease, |
| | | | | | | | | | | | | | | | autosomal 1) |
| K220 | D86974_at | D86974 | PASS | 9 | 239.22 | PASS | 13 | 9 | 73.38 | 3.26 | 3.26 | K1AA0220 | | | |
| K109_CLAS1 | D63475_at | D63475 | PASS | 8 | 45.88 | PASS | 13 | 8 | 14.15 | 3.24 | 3.24 | CLAPM1 | 3q28 | clathrin-associated | clathrin-associated/ |
| | | | | | | | | | | | | | | assembly/adaptor | assembly/adaptor |
| | | | | | | | | | | | | | | protein, medium 1 | protein, medium 1 |
| TSC2 | L48546_at | L48546 | PASS | 9 | 30.44 | PASS | 7 | 9 | 9.43 | 3.23 | 3.23 | TSC2 | 16p13.3 | tuberous sclerosis 2 | tuberous sclerosis 2 |
| EDR2 | U89278_at | U89278 | PASS | 8 | 25.25 | PASS | 12 | 8 | 7.83 | 3.22 | 3.22 | EDR2 | | early development regulator | early development regulator |
| | | | | | | | | | | | | | | 2 (homolog of poly- | 2 (homolog of poly- |
| | | | | | | | | | | | | | | homeotic 2) | homeotic 2) |
| M34996_s_at | M34996_s_at | M34996 | PASS | 9 | 80.56 | PASS | 13 | 9 | 25.08 | 3.21 | 3.21 | | | cell surface glycoprotein | |
| U59632_s_at | U59632_s_at | U59632 | PASS | 9 | 97.22 | PASS | 13 | 9 | 30.54 | 3.18 | 3.18 | PNUTL1 | 22q11.2 | peanut (Drosphila)-like 1 | peanut (Drosphila)-like 1 |
| UHX1 | U44839_at | U44839 | PASS | 9 | 60.22 | PASS | 13 | 9 | 18.92 | 3.18 | 3.18 | USP11 | Xp21.2-p11.2 | Ubiquitin carboxyl-terminal | Ubiquitin specific protease |
| | | | | | | | | | | | | | | hydrolase, X-linked | 11 |
| UROD | X89267_at | X89267 | PASS | 5 | 47.60 | PASS | 8 | 5 | 15.25 | 3.12 | 3.12 | | | uroporphymogen | |
| | | | | | | | | | | | | | | | decarboxylase |
| PLCB2 | M95678_at | M95678 | PASS | 9 | 84.00 | PASS | 12 | 9 | 26.92 | 3.12 | 3.12 | PLCB2 | 15q15 | phospholipase C, beta 2 | phospholipase C, beta 2 |
| BST2 | D28137_at | D28137 | PASS | 8 | 51.13 | PASS | 13 | 8 | 16.38 | 3.12 | 3.12 | BST2 | 19p13.2 | bone marrow stromal cell | bone marrow stromal cell |
| | | | | | | | | | | | | | | antigen 2 | antigen 2 |
| NFER2 | S77763_at | S77763 | PASS | 9 | 32.33 | PASS | 11 | 9 | 10.36 | 3.12 | 3.12 | nuclear | | nuclear factor erythroid 2 | basic leucine zipper protein; |
| | | | | | | | | | | | | erythroid 2 | | isoform f | This sequence comes from |
| | | | | | | | | | | | | isoform f, | | | FIG. 1; transcription |
| | | | | | | | | | | | | transcription | | | factor fNF-E2 |
| | | | | | | | | | | | | factor | | | |
| | | | | | | | | | | | | fNF-E2 | | | |
| EBVIP | U19261_at | U19261 | PASS | 6 | 22.67 | PASS | 7 | 6 | 7.29 | 3.11 | 3.11 | | | Epstein-Barr virus-induced | EBV induced protein |
| 28SRNAM | AFFX-M278 | AFFX-M27 | PASS | 5 | 91.00 | PASS | 7 | 5 | 29.29 | 3.11 | 3.11 | | | | |
| GSTZ1 | U86529_at | U86529 | PASS | 9 | 25.56 | PASS | 11 | 9 | 8.27 | 3.09 | 3.09 | GSTZ1 | 14q24.3 | glutathione S-transferase | glutathione S-transferase |
| | | | | | | | | | | | | | | Zeta 1 | Zeta 1 |
| CD151 | D29963_at | D29963 | PASS | 8 | 31.13 | PASS | 7 | 8 | 10.14 | 3.07 | 3.07 | CD151 | 11p15.5 | CD151 antigen | CD151 antigen |
| SAT_rna1 | U40369_rna1 | U40369 | PASS | 9 | 37.67 | PASS | 13 | 9 | 12.31 | 3.06 | 3.06 | SAT | Xp22.1 | spermidine/spermine N1- | spermidine-spermine N1- |
| | | | | | | | | | | | | | | acetyltransferase | acetyltransferase |
| CLU | M63379_at | M63379 | PASS | 9 | 222.78 | PASS | 13 | 9 | 72.85 | 3.06 | 3.06 | CLU | Sp21-p12 | clusterin (complement lysis | clusterin (complement lysis |
| | | | | | | | | | | | | | | inhibitor, SP-40,40, | inhibitor, SP-40,40 |
| | | | | | | | | | | | | | | sulfated glycoprotein 2, | sulfated glycoprotein 2, |
| | | | | | | | | | | | | | | testosterone-repressed | testosterone-repressed |
| | | | | | | | | | | | | | | prostate message 2, | prostate message 2, |
| | | | | | | | | | | | | | | apolipoprotein J) | apolipoprotein J) |
| HMG1 | D63874_at | D63874 | PASS | 9 | 83.22 | PASS | 13 | 9 | 27.31 | 3.05 | 3.05 | HMG1 | 13q12 | high-mobility group | high-mobility group |
| | | | | | | | | | | | | | | (nonhistone chromosomal) | (nonhistone chromosomal) |
| | | | | | | | | | | | | | | protein 1 | protein 1 |
| DEFA1 | M26602_at | M26602 | PASS | 7 | 185.43 | PASS | 11 | 7 | 61.09 | 3.04 | 3.04 | DEFA1 | 8p23-2p23.1 | defensin, alpha 1, myeloid- | defensin, alpha 1, myeloid- |
| | | | | | | | | | | | | | | related sequence | related sequence |
| FCGR1A | J004162_at | J04162 | PASS | 9 | 4656 | PASS | 13 | 9 | 15.38 | 3.03 | 3.03 | FCGR3A | 1q23 | | Fe fragment of IgG, low |
| | | | | | | | | | | | | | | | affinity IIIa, receptor |
| | | | | | | | | | | | | | | | for (CD16) |
| M32304_s_at | M32304_s_at | M32304 | PASS | 8 | 26.75 | PASS | 13 | 8 | 8.85 | 3.02 | 3.02 | TIMP2 | 17q25 | tissue inhibitor of | tissue inhibitor of |
| | | | | | | | | | | | | | | metalloproteinase 2 | metalloproteinase 2 |
| LSP1 | M33552_at | M33552 | PASS | 9 | 48.11 | PASS | 13 | 9 | 15.92 | 3.02 | 3.02 | LSP1 | | | lymphocte-specific |
| | | | | | | | | | | | | | | | protein 1 (LSP1) |
| U83239_s_at | U83239_s_at | U83239 | PASS | 6 | 34.33 | PASS | 11 | 6 | 11.36 | 3.02 | 3.02 | | | CC chemokine STCP-1 | |
| GSTH | U90313_at | U90313 | PASS | 9 | 43.22 | PASS | 13 | 9 | 14.31 | 3.02 | 3.02 | GSTTLp28 | | glutathione-S-transferase | glutathione-S-transferase |
| | | | | | | | | | | | | | | like | like |
| IGLT1 | U82275_at | U82275 | PASS | 8 | 27.88 | PASS | 12 | 8 | 9.25 | 3.01 | 3.01 | | | immunoglobulin-like | ILT1; Ig-superfamily |
| | | | | | | | | | | | | | | transcript 1 | member |
| NRGN_rna1 | X99076_rna1 | X99076 | PASS | 9 | 230.11 | PASS | 13 | 9 | 76.54 | 3.01 | 3.01 | NRGN | | neurogranin | |
| UBA52 | M26880_at | M26880 | PASS | 9 | 198.00 | PASS | 13 | 9 | 66.31 | 2.99 | 2.99 | UBA52 | 19p13.1-p12 | ubiquitin A-52 residue | ubiquitin A-52 residue |
| | | | | | | | | | | | | | | ribosomal protein fusion | ribosomal protein fusion |
| | | | | | | | | | | | | | | | product 1 |
| TMEM1 | D26579_at | D26579 | PASS | 9 | 31.56 | PASS | 13 | 9 | 10.62 | 2.97 | 2.97 | ADAM8 | 10q26.3 | a disintegrin and metallo- | a disintegrain and metallo- |
| | | | | | | | | | | | | | | protease domain 8 | protease domain 8 |
| GP1 | K03515_at | K03515 | PASS | 9 | 35.78 | PASS | 13 | 9 | 12.08 | 2.96 | 2.96 | GP1 | 19q13.1 | glucose phosphate | glucose phosphate |
| | | | | | | | | | | | | | | isomerase | isomerase |
| TYL | X99688_at | X99688 | PASS | 9 | 23.44 | PASS | 12 | 9 | 7.92 | 2.96 | 2.96 | TYL | | | |
| UBE1L | L13852_at | L13852 | PASS | 9 | 54.78 | PASS | 13 | 9 | 18.62 | 2.94 | 2.94 | UBE1L | 3p21 | ubiquitin-activating | ubiquitin-activating |
| | | | | | | | | | | | | | | enzyme E1, like | enzyme E1, like |
| KRT1_rna1 | M98776_rna1 | M98776 | PASS | 7 | 19.29 | PASS | 9 | 7 | 6.56 | 2.94 | 2.94 | KRT1 | | keratin 1 | |
| K45_YKL | D28476_at | D28476 | PASS | 9 | 27.44 | PASS | 12 | 9 | 9.33 | 2.94 | 2.94 | TRIP12 | | thyroid hormone receptor | |
| | | | | | | | | | | | | | | | interactor 12 |
| HCFC1 | L20010_at | L20010 | PASS | 8 | 26.13 | PASS | 13 | 8 | 8.92 | 2.93 | 2.93 | | | | |
| SLC9A1 | S68616_at | S68616 | PASS | 5 | 20.20 | PASS | 10 | 5 | 6.90 | 2.93 | 2.93 | SLC9A1 | 1p36.1-p35 | Na+/H+ exchanger | solute carrier family 9 |
| | | | | | | | | | | | | | | NHE-1 isoform | (sodium/hydrogen |
| | | | | | | | | | | | | | | | exchanger), isoform 1 |
| | | | | | | | | | | | | | | | (antiporter, Na+/H+, |
| | | | | | | | | | | | | | | | amiloride sensitive) |
| SCYA5 | M21121_at | M21121 | PASS | 9 | 156.78 | PASS | 13 | 9 | 53.69 | 292 | 2.92 | SCYA5 | 17q11.2-q12 | small inducible cytokine | small inducible cytokine |
| | | | | | | | | | | | | | | A5 (RANTES) | A5 (RANTES) |
| PRKMK3 | D87116_at | D87116 | PASS | 9 | 32.89 | PASS | 11 | 9 | 11.27 | 2.92 | 2.92 | PRKMK3 | 17q11.2 | protein kinase, mitogen- | protein kinase, mitogen- |
| | | | | | | | | | | | | | | activated, kinase 3 | activated, kinase 3 |
| | | | | | | | | | | | | | | (MAP kinase kinase 3) | (MAP kinase kinase 3) |
| CCND3 | M92287_at | M92287 | PASS | 9 | 68.33 | PASS | 13 | 9 | 23.62 | 2.89 | 2.89 | CCND3 | 6p21 | cyclin D3 | cyclin D3 |
| SMN1_rna2 | U80017_rna2 | U80017 | PASS | 8 | 18.38 | PASS | 11 | 8 | 6.36 | 2.89 | 2.89 | btf2p44 | | basic transcription | NAIP |
| | | | | | | | | | | | | | | factor 2 p44 | |
| PLCG2H | U45975_at | U45975 | PASS | 6 | 23.50 | PASS | 7 | 6 | 8.14 | 2.89 | 2.89 | | | | phosphatidylmositol |
| | | | | | | | | | | | | | | | (4,5)bisphosphate 5- |
| | | | | | | | | | | | | | | | phosphatase homolog |
| X74874_rna2 | X74874_rna2 | X74874 | PASS | 8 | 19.75 | PASS | 13 | 8 | 6.85 | 2.88 | 2.88 | | | RNA polymerase II | |
| | | | | | | | | | | | | | | largest subunit | |
| M36118_s_at | M36118_s_at | M36118 | PASS | 8 | 33.63 | PASS | 12 | 8 | 11.67 | 2.88 | 2.88 | GZMB | 14q11.2 | granzyme B (granzyme | granzyme B (granzyme |
| | | | | | | | | | | | | | | 2, cytotoxic T- | 2, cytotoxic T- |
| | | | | | | | | | | | | | | lymphocyte-associated | lymphocyte-associated |
| | | | | | | | | | | | | | | serine esterase 1) | serine esterase 1) |
| IMPDH1 | J05272_at | J05272 | PASS | 9 | 31.44 | PASS | 13 | 9 | 10.92 | 2.88 | 2.88 | IMPDH1 | 7q31.3-q32 | IMP (inosine mono- | IMP (inosine mono- |
| | | | | | | | | | | | | | | phosphate) dehydrogenase | phosphate) dehydrogenase |
| | | | | | | | | | | | | | | 1 | 1 |
| S40719_s_at | S40719_s_at | S40719 | PASS | 9 | 19.89 | PASS | 11 | 9 | 6.91 | 2.88 | 2.88 | GFAP | 17q21 | glial fibrillary acidic | glial fibrillary acidic |
| | | | | | | | | | | | | | | protein | protein |
| NAP1L4 | U77456_at | U77456 | PASS | 6 | 29.50 | PASS | 12 | 6 | 10.25 | 2.88 | 2.88 | | | nucleosome assembly | hNAP2 |
| | | | | | | | | | | | | | | protein 2 | |
| E_ZNF162 | L49380_at | L49380 | PASS | 9 | 46.44 | PASS | 13 | 9 | 16.15 | 2.88 | 2.88 | ZNF162 | 11q13 | zinc finger protein 162 | zinc finger protein 162 |
| S100A12 | D83657_at | D83657 | PASS | 9 | 65.44 | PASS | 13 | 9 | 22.77 | 2.87 | 2.87 | | | CAAF1 (calcium-binding | |
| | | | | | | | | | | | | | | protein in amniotic fluid | |
| K56 | D29954_at | D29954 | PASS | 8 | 18.88 | PASS | 7 | 8 | 6.57 | 2.87 | 2.87 | KIAA0056 | | | |
| E_DDX11 | U75968_at | U75968 | PASS | 9 | 20.56 | PASS | 12 | 9 | 7.17 | 2.87 | 2.87 | CHLR1 | CHL1 protein | | |
| ORP150 | U65785_at | U65785 | PASS | 9 | 33.67 | PASS | 12 | 9 | 11.75 | 2.87 | 2.87 | | | 150 kDa oxygen-regulated | |
| | | | | | | | | | | | | | | protein ORP150 | |
| ARF5 | M57567_at | M57567 | PASS | 8 | 46.00 | PASS | 13 | 8 | 16.15 | 2.85 | 2.85 | ARF5 | 7q31.3 | ADP-ribosylation factor 5 | ADP-ribosylation factor 5 |
| S69272_s_at | S69272_s_at | S69272 | PASS | 9 | 24.67 | PASS | 13 | 9 | 8.69 | 2.84 | 2.84 | P16 | 6p25 | protease inhibitor 6 | protease inhibitor 6 |
| | | | | | | | | | | | | | | (placental thrombin | (placental thrombin |
| | | | | | | | | | | | | | | inhibitor) | inhibitor) |
| AB002356_s | AB002356_s | AB002356 | PASS | 9 | 31.67 | PASS | 12 | 9 | 11.17 | 2.84 | 2.84 | MADD | 11p11.21- | MAP-kinase activating | MAP-kinase activating |
| | | | | | | | | | | | | | p11.22 | death domain | death domain |
| CSF1 | HG1155-HT4 | HG1155-HT | PASS | 8 | 27.63 | PASS | 9 | 8 | 9.78 | 2.83 | 2.83 | | | | |
| RGS2 | L13391_at | L13391 | PASS | 9 | 60.33 | PASS | 13 | 9 | 21.38 | 2.82 | 2.82 | RGS2 | 1q31 | regulator of G-protein | regulator of G-protein |
| | | | | | | | | | | | | | | signalling 2,24 kD | signalling 2, 24 kD |
| UP | X90858_at | X90858 | PASS | 9 | 21.44 | PASS | 13 | 9 | 7.62 | 2.82 | 2.82 | UP | 7 | uridine phosphorylase | uridine phosphorylase |
| K250 | D87437_at | D87437 | PASS | 9 | 19.33 | PASS | 9 | 9 | 6.89 | 2.81 | 2.81 | KIAA0250 | | | KIAA0250 gene product |
| CNP_cds1 | D13146_cds1 | D13146 | PASS | 9 | 49.89 | PASS | 13 | 9 | 17.85 | 2.80 | 2.80 | | | 2′,3′-cyclic-nucleotide 3′- | alternative splicing |
| | | | | | | | | | | | | | | phosphodiesterase (CNPT) | |
| CDA | L27943_at | L27943 | PASS | 6 | 32.33 | PASS | 10 | 6 | 11.60 | 2.79 | 2.79 | CDA | 1p36.2-p35 | cytidine deaminase | cytidine deaminase |
| FAST | X86779_at | X86779 | PASS | 9 | 20.33 | PASS | 10 | 9 | 7.30 | 2.79 | 2.79 | fast | | FAST kinase | |
| X59932_s_at | X59932_s_at | X59932 | PASS | 9 | 62.44 | PASS | 13 | 9 | 22.46 | 2.78 | 2.78 | CSK | 15q23-q25 | c-src tyrosine kinase | c-src tyrosine kinase |
| MAZ | M94046_at | M94046 | PASS | 9 | 29.67 | PASS | 13 | 9 | 10.69 | 2.77 | 2.77 | | | | |
| DF | M84526_at | M84526 | PASS | 5 | 43.40 | PASS | 12 | 5 | 15.67 | 2.77 | 2.77 | DF | | D component of | D component of |
| | | | | | | | | | | | | | | complement (adipsin) | complement (adipsin) |
| PRKM3 | D28915_at | D28915 | PASS | 7 | 16.00 | PASS | 10 | 7 | 5.80 | 2.76 | 2.76 | | | hepatitis C-associated | |
| | | | | | | | | | | | | | | microtubular aggregate | |
| | | | | | | | | | | | | | | protein p44 | |
| CD33 | M23197_at | M23197 | PASS | 8 | 21.00 | PASS | 13 | 8 | 7.62 | 2.76 | 2.76 | CD33 | 19q13.3 | CD33 antigen (gp67) | CD33 antigen (gp67) |
| D78577_s_at | D78577_s_at | D78577 | PASS | 9 | 85.67 | PASS | 13 | 9 | 31.08 | 2.76 | 2.76 | | | 14-3-3 protein eta chain | |
| BRF2 | X78992_at | X8992 | PASS | 8 | 64.88 | PASS | 13 | 8 | 23.54 | 2.76 | 2.76 | ERF-2 | | | |
| CLTA | M20471_at | M20471 | PASS | 9 | 73.56 | PASS | 13 | 9 | 26.69 | 2.76 | 2.76 | CLTA | 12q23-q24 | clathrin, light polypeptide | clathrin, light polypeptide |
| | | | | | | | | | | | | | | (Lea) | (Lea) |
| HG2868-HT3 | HG2868-HT3 | HG2868-HT | PASS | 7 | 18.57 | PASS | 12 | 7 | 6.75 | 2.75 | 2.75 | | | | |
| MCL1 | L08246_at | L08246 | PASS | 9 | 88.67 | PASS | 13 | 9 | 32.23 | 2.75 | 2.75 | MCL1 | 1q21 | | myeloid cell leukemia |
| | | | | | | | | | | | | | | | (BCL2-related) |
| S100A11 | D38583_at | D38583 | PASS | 9 | 81.00 | PASS | 13 | 9 | 29.54 | 2.74 | 2.74 | | | | |
| TNFR2 | M32315_at | M32315 | PASS | 9 | 67.44 | PASS | 13 | 9 | 24.62 | 2.74 | 2.74 | TNFRSF1B | 1p36.3-p36.2 | tumor necrosis factor | tumor necrosis factor |
| | | | | | | | | | | | | | | receptor 2 (75 kD) | superfamily, member 1B |
| GNG10 | U31383_at | U31383 | PASS | 9 | 18.33 | PASS | 13 | 9 | 6.69 | 2.74 | 2.74 | GNG10 | | guanine nucleodide | guanine nucleotide |
| | | | | | | | | | | | | | | binding protein 10 | binding protein 10 |
| D38251_s_at | D38251_s_at | D38251 | PASS | 8 | 30.75 | PASS | 13 | 8 | 11.23 | 2.74 | 2.74 | PLLR2E | 19p13.3 | polymerase (RNA) II | polymerase (RNA) II |
| | | | | | | | | | | | | | | (DNA directed) poly- | (DNA directed) poly- |
| | | | | | | | | | | | | | | peptide E (25 kD) | peptide E (25 kD) |
| K50_K41 | D30758_at | D30758 | PASS | 9 | 61.89 | PASS | 13 | 9 | 22.62 | 2.74 | 2.74 | KIAA0050 | | | KIAA0050 gene product |
| M38449_s_at | M38449_s_at | M38449 | PASS | 6 | 33.50 | PASS | 8 | 6 | 12.25 | 2.73 | 2.73 | TGF-beta | | transforming growth factor- | |
| GT197 | L38932_at | L38932 | PASS | 9 | 40.33 | PASS | 13 | 9 | 14.77 | 2.73 | 2.73 | BECN1 | | beclin 1 (coiled-coil, | beclin 1 (coiled-coil, |
| | | | | | | | | | | | | | | myosin-like BCL2- | myosin-like BCL2- |
| | | | | | | | | | | | | | | interacting protein) | interacting protein) |
| AMPD2_cds1 | M91029_cds2 | M91029 | PASS | 9 | 30.3 | PASS | 13 | 9 | 11.15 | 2.72 | 2.72 | AMPD2 | 1p13.3 | adenosine monophosphate | adenosine monophosphate |
| | | | | | | | | | | | | | | deaminase 2 (isoform L) | deaminase 2 (isoform L) |
| RABGGTA | Y08200_at | Y08200 | PASS | 9 | 23.56 | PASS | 12 | 9 | 8.67 | 2.72 | 2.72 | RABGGTA | 14q11.2 | Rab geranylgeranyl- | Rab geranylgeranyl- |
| | | | | | | | | | | | | | | transferase, alpha subunit | transferase, alpha subunit |
| Y08682_rna1 | Y08682_rna1 | Y08682 | PASS | 9 | 13.56 | PASS | 8 | 9 | 5.00 | 2.71 | 2.71 | CPT1B | | carnitine palmitoyl- | type I |
| | | | | | | | | | | | | | | transferase 1 | |
| MYH9 | M31013_at | M31013 | PASS | 9 | 149.78 | PASS | 13 | 9 | 55.38 | 2.70 | 2.70 | MYH9 | 22q12.3-q13.1 | | myosin, heavy polypeptide |
| | | | | | | | | | | | | | | | 9, non-muscle |
| D00749_s_at | D00749_s_at | D00748 | PASS | 9 | 69.89 | PASS | 13 | 9 | 25.85 | 2.70 | 2.70 | | | CD7 antigen | |
| U65416_rna1 | U65416_rna1 | U65416 | PASS | 9 | 17.33 | PASS | 12 | 9 | 6.42 | 2.70 | 2.70 | MICB | | MHC class I molecule | MHC class I chain-related |
| | | | | | | | | | | | | | | | gene B; cDNA sequence |
| | | | | | | | | | | | | | | | deposited under GenBank |
| | | | | | | | | | | | | | | | Accession Number |
| | | | | | | | | | | | | | | | X91625 |
| Z22951_rna1 | Z22951_rna1 | Z22951 | PASS | 5 | 18.60 | PASS | 10 | 5 | 6.90 | 2.70 | 2.70 | p65 | | p65 subunit of transcription | |
| | | | | | | | | | | | | | | factor NF-kappaB | |
| PRCC_rna1 | X99720_rna1 | X99720 | PASS | 7 | 22.43 | PASS | 9 | 7 | 8.33 | 2.69 | 2.69 | TPRC | | | |
| HG2238-HT2 | HG2238-HT2 | HG2238-HT | PASS | 9 | 25.67 | PASS | 13 | 9 | 9.54 | 2.69 | 2.69 | | | | |
| SLC2A3 | M20681_at | M20681 | PASS | 9 | 26.00 | PASS | 12 | 9 | 9.67 | 2.69 | 2.69 | SLC2A3 | 12p13.3 | | solute carrier family 2 |
| | | | | | | | | | | | | | | | (facilitated glucose |
| | | | | | | | | | | | | | | | transporter), member 3 |
| FCGRT | U12255_at | U12255 | PASS | 9 | 78.56 | PASS | 13 | 9 | 29.23 | 2.69 | 2.69 | FCGRT | 19q13.3 | Fc fragment of IgG, | Fc fragment of IgG, |
| | | | | | | | | | | | | | | receptor, transporter, alpha | receptor, transporter, alpha |
| MAPT | HG2566-HT4 | HG2566-HT | PASS | 8 | 28.38 | PASS | 7 | 8 | 10.57 | 2.68 | 2.68 | | | | |
| E_IFNGR2 | U05875_at | U05875 | PASS | 8 | 34.88 | PASS | 8 | 8 | 13.00 | 2.68 | 2.68 | | | AF-1 | second chain of the receptor |
| TCFL1 | D43642_at | D43642 | PASS | 9 | 38.22 | PASS | 13 | 9 | 14.31 | 2.67 | 2.67 | YL-1 | | YL-1 protein | Nuclear protein with DNA- |
| | | | | | | | | | | | | | | | binding ability |
| U66711_rna1 | U66711_rna1 | U66711 | PASS | 8 | 48.75 | PASS | 12 | 8 | 18.25 | 2.67 | 2.67 | LY6E | 8q24.3 | lymphocyte antigen 6 | lymphocyte antigen 6 |
| | | | | | | | | | | | | | | complex, locus E | complex, locus E |
| HVEM | U70321_at | U70321 | PASS | 9 | 28.11 | PASS | 13 | 9 | 10.54 | 2.67 | 2.67 | TNPRSF14 | 1p36.3-p36.2 | tumor necrosis factor | tumor necrosis factor |
| | | | | | | | | | | | | | | receptor superfamily, | receptor superfamily, |
| | | | | | | | | | | | | | | member 14; herpes virus | member 14; (herpes virus |
| | | | | | | | | | | | | | | entry mediator | entry mediator) |
| TPR2 | U46571_at | U46571 | PASS | 9 | 17.78 | PASS | 12 | 9 | 6.67 | 2.67 | 2.67 | TTC2 | 17q11.2 | tetratricopeptide repeat | tetratricopeptide repeat |
| | | | | | | | | | | | | | | domain 2 | domain 2 |
| GARS | U09587_at | U09587 | PASS | 9 | 29.67 | PASS | 13 | 9 | 11.15 | 2.66 | 2.66 | | | glycyl-tRNA synthetase | |
| ARAF1 | U01337_at | U01337 | PASS | 9 | 32.67 | PASS | 12 | 9 | 12.33 | 2.65 | 2.65 | A-RAP-1 | | Ser/Thr protein kinase | cytoplasmic |
| ISGF3G | M87503_at | M87503 | PASS | 8 | 52.50 | PASS | 13 | 8 | 19.85 | 2.65 | 2.65 | ISGF3- | | IFN-alpha responsive | |
| | | | | | | | | | | | | gamma | | transcription factor | |
| P1 | K01396_at | K01396 | PASS | 9 | 139.78 | PASS | 13 | 9 | 52.85 | 2.64 | 2.64 | P1 | 14q32.1 | protease inhibitor 1 (anti- | protease inhibitor 1 (anti- |
| | | | | | | | | | | | | | | elastase), alpha-1- | elastase), alpha-1- |
| | | | | | | | | | | | | | | antitrypsin | antitrypsin |
| BTG2 | U72649_at | U72649 | PASS | 9 | 4033 | PASS | 12 | 9 | 15.25 | 2.64 | 2.64 | BTG2 | | BTG2 | rat PC3 and murine |
| | | | | | | | | | | | | | | | TIS21 genes homolog |
| TXNRD1 | U78678_at | U78678 | PASS | 8 | 20.25 | PASS | 9 | 8 | 7.67 | 2.64 | 2.64 | | | thioredoxin | |
| CSNK2A2 | M55268_at | M55268 | PASS | 9 | 17.33 | PASS | 7 | 9 | 6.57 | 2.64 | 2.64 | CSNK2A2 | 16p13.3-p13.2 | casein kinase 2, alpha | casein kinase 2, alpha |
| | | | | | | | | | | | | | | prime polypeptide | prime polypeptide |
| ARHG | X61587_at | X61587 | PASS | 9 | 51.89 | PASS | 13 | 9 | 19.69 | 2.63 | 2.63 | ARHG | 11p15.5-p15.4 | ras homolog gene family, | ras homolog gene family, |
| | | | | | | | | | | | | | | member G (rho G) | member G (rho G) |
| IRF3 | Z56281_at | Z56281 | PASS | 9 | 25.11 | PASS | 13 | 9 | 9.54 | 2.63 | 2.63 | IRP3 | 19q13.3-q13.4 | interferon regulatory factor | interferon regulatory factor |
| | | | | | | | | | | | | | | 3 | 3 |
| HEM1 | M58285_at | M58285 | PASS | 9 | 38.44 | PASS | 13 | 9 | 14.62 | 2.63 | 2.63 | | | membrane-associated | |
| | | | | | | | | | | | | | | protein HEM-1 | |
| NRAMP1 | D50402_at | D50402 | PASS | 9 | 19.11 | PASS | 11 | 9 | 7.27 | 2.63 | 2.63 | NRAMP1 | 2q35 | Nramp | natural resistance- |
| | | | | | | | | | | | | | | | associated macrophage |
| | | | | | | | | | | | | | | | protein 1 (might include |
| | | | | | | | | | | | | | | | Leishmaniasis) |
| CLAPB1 | M34175_at | M34175 | PASS | 9 | 28.22 | PASS | 12 | 9 | 10.75 | 2.63 | 2.63 | CLAPB1 | 17q11.2-q12 | clathrin-associated/ | clathrin-associated/ |
| | | | | | | | | | | | | | | assembly/adaptor | assembly/adaptor |
| | | | | | | | | | | | | | | protein, large, beta 1 | protein, large, beta 1 |
| ZFP77 | HG4332-HT4 | HG4332-HT | PASS | 8 | 14.63 | PASS | 7 | 8 | 5.57 | 2.63 | 2.63 | | | | |
| K151_SPK1 | D63485_at | D63485 | PASS | 9 | 18.89 | PASS | 10 | 9 | 7.20 | 2.62 | 2.62 | KIAA0151 | | | KIAA0151 gene product |
| K226 | D86979_at | D86979 | PASS | 9 | 20.56 | PASS | 13 | 9 | 7.85 | 2.62 | 2.62 | KIAA0226 | | | KIAA0226 gene product |
| BTN_rna1 | U97502_rna1 | U97502 | PASS | 6 | 16.50 | PASS | 13 | 6 | 6.31 | 2.62 | 2.62 | BT3.3 | | butyrophilin | |
| L32831_s_at | L32831_s_at | L32831 | PASS | 5 | 18.00 | PASS | 9 | 5 | 6.89 | 2.61 | 2.61 | | | G protein-coupled receptor | |
| | | | | | | | | | | | | | | GPR3 | |
| FKBP4 | M88279_at | M88279 | PASS | 9 | 23.11 | PASS | 13 | 9 | 8.85 | 2.61 | 2.61 | FKBP4 | | FK506-binding protein 4 | FK506-binding protein 4 |
| | | | | | | | | | | | | | | (59 kD) | (59 kD) |
| CTSD | M63138_at | M63138 | PASS | 9 | 82.89 | PASS | 12 | 9 | 31.75 | 2.61 | 2.61 | CTSD | 11p15.5 | cathepsin D (lysosomal | cathepsin D (lysosomal |
| | | | | | | | | | | | | | | aspartyl protease) | aspartyl protease) |
| HG2815-HT4 | HG2815-HT4 | HG2815-HT | PASS | 9 | 360.22 | PASS | 13 | 9 | 138.00 | 2.61 | 2.61 | | | | |
| L13939_s_at | L13939_s_at | L13939 | PASS | 8 | 24.88 | PASS | 13 | 8 | 9.54 | 2.61 | 2.61 | ADTB1 | 22q12 | adaptin, beta 1 (beta prime) | adaptin, beta 1 (beta prime) |
| AOAH | M62840_at | M62840 | PASS | 8 | 27.50 | PASS | 9 | 8 | 10.56 | 2.61 | 2.61 | AOAH | 7p14-p12 | acyloxyacyl hydrolase | acyloxyacyl hydrolase |
| | | | | | | | | | | | | | | (neutrophil) | (neutrophil) |
| TPR1 | U46570_at | U46570 | PASS | 9 | 41.67 | PASS | 13 | 9 | 16.08 | 2.59 | 2.59 | TTC1 | 5q32-q33.2 | tetratricopeptide repeat | tetratricopeptide repeat |
| | | | | | | | | | | | | | | domain 1 | domain 1 |
| TUBA1 | X01703_at | X01703 | PASS | 9 | 37.67 | PASS | 13 | 9 | 14.54 | 2.59 | 2.59 | | | alpha-tubulin | |
| C5R1 | M62505_at | M62505 | PASS | 8 | 25.00 | PASS | 12 | 8 | 9.67 | 2.59 | 2.59 | C5R1 | 19q13.3-q13.4 | complement component 5 | complement component 5 |
| | | | | | | | | | | | | | | receptor 1 (C5a ligand) | receptor 1 (C5 ligand) |
| U43185_s_at | U43185_s_at | U43185 | PASS | 9 | 27.56 | PASS | 12 | 9 | 10.67 | 2.58 | 2.58 | STAT5A | 17q11.2 | signal transducer and | signal transducer and |
| | | | | | | | | | | | | | | activator of transcription | activator of transcription |
| | | | | | | | | | | | | | | 5A | 5A |
| AARS | D32050_at | D32050 | PASS | 8 | 19.38 | PASS | 12 | 8 | 7.50 | 2.58 | 2.58 | AARS | 16q22 | alanyl-tRNA synthetase | alanyl-tRNA synthetase |
| SREBF1 | U00968_at | U00968 | PASS | 6 | 24.67 | PASS | 7 | 6 | 9.57 | 2.58 | 2.58 | SREBF1 | 17p1.2 | sterol regulatory element | sterol regulatory element |
| | | | | | | | | | | | | | | binding transcription | binding transcription |
| | | | | | | | | | | | | | | factor 1 | factor 1 |
| G1P2 | M13755_at | M13755 | PASS | 7 | 30.71 | PASS | 13 | 7 | 11.92 | 2.58 | 2.58 | ISG15 | 1 | | interferon-stimulated |
| | | | | | | | | | | | | | | | protein, 15 kDa |
| BCAT2 | U62739_at | U62739 | PASS | 9 | 18.78 | PASS | 10 | 9 | 7.30 | 2.57 | 2.57 | BCAT2 | 19 | branched chain | branched chain |
| | | | | | | | | | | | | | | aminotransferase 2, | aminotransferase 2, |
| | | | | | | | | | | | | | | | mitochondrial |
| DCTD | L39874_at | L39874 | PASS | 8 | 26.38 | PASS | 11 | 8 | 10.27 | 2.57 | 2.57 | DCTD | | DCMP deaminase | dCMP deaminase |
| K15_PPMIA | D13640_at | D13640 | PASS | 9 | 29.00 | PASS | 12 | 9 | 11.33 | 2.56 | 2.56 | KIAA0015 | | | KIAA0015 gene product |
| RTP | D87953_at | D87953 | PASS | 9 | 39.56 | PASS | 13 | 9 | 15.46 | 2.56 | 2.56 | GC4 | | RTP | |
| PXN | U14588_at | U14588 | PASS | 9 | 39.11 | PASS | 13 | 9 | 15.31 | 2.55 | 2.55 | PXN | 12q24 | paxillin | paxillin |
| KAP1_TIFIE | U95040_at | U95040 | PASS | 9 | 44.00 | PASS | 13 | 9 | 17.31 | 2.54 | 2.54 | | | hKAP1/TIF1B | |
| NRBTK | L20773_at | L20773 | PASS | 9 | 25.56 | PASS | 13 | 9 | 10.08 | 2.54 | 2.54 | | | | |
| AJ000099_s_{—} | AJ000099_s_{—} | AJ000099 | PASS | 7 | 28.57 | PASS | 11 | 7 | 11.27 | 2.53 | 2.53 | HYAL2 | 3p21.3 | hyaluronoglucosaminidase | hyaluronoglucosaminidase |
| | | | | | | | | | | | | | | 2 | 2 |
| BZRP | L21954_at | L21954 | PASS | 9 | 127.89 | PASS | 13 | 9 | 50.46 | 2.53 | 2.53 | BZRP | 22q13.3 | benzodiazapine receptor | benzodiazapine receptor |
| | | | | | | | | | | | | | | peripheral | peripheral |
| HUK5 | U67963_at | U67963 | PASS | 9 | 18.89 | PASS | 11 | 9 | 7.45 | 2.53 | 2.53 | HU-K5 | | lysophospholinase homolog | |
| YF5 | U84569_at | U84569 | PASS | 8 | 24.88 | PASS | 13 | 8 | 9.85 | 2.53 | 2.53 | | | YF5 | similar to A2 encoded by |
| | | | | | | | | | | | | | | | GenBank Accession |
| | | | | | | | | | | | | | | | Number U84570 and to |
| | | | | | | | | | | | | | | | sequence with GenBank |
| | | | | | | | | | | | | | | | Accession Number |
| | | | | | | | | | | | | | | | AC000020 |
| STX5A | U26648_at | U26648 | PASS | 6 | 21.33 | PASS | 9 | 6 | 8.44 | 2.53 | 2.53 | STX5A | | syntaxin 5A | syntaxin 5A |
| X65784_s_at | X65784_s_at | X65784 | PASS | 8 | 21.88 | PASS | 12 | 8 | 8.67 | 2.52 | 2.52 | CMAR | 16q | | cell matrix adhesion |
| | | | | | | | | | | | | | | | regulator |
| SFCC13 | L10910_at | L10910 | PASS | 9 | 16.11 | PASS | 13 | 9 | 6.38 | 2.52 | 2.52 | CC1.3 | 20 | splicing factor (CC1.3) | splicing factor (CC1.3) |
| K79_CHR7 | D38555_at | D38555 | PASS | 9 | 21.33 | PASS | 10 | 9 | 8.50 | 2.51 | 2.51 | KIAA0079 | 10 | Sec24p, S. Cerevisiae, | Sec24p, S. Cerevisiae, |
| | | | | | | | | | | | | | | homolog of | homolog of |
| E_A9A2BRD | U00952_at | U00952 | PASS | 5 | 17.80 | PASS | 10 | 5 | 7.10 | 2.51 | 2.51 | | | | |
| LAG2 | M85276_at | M85276 | PASS | 9 | 138.22 | PASS | 13 | 9 | 55.15 | 2.51 | 2.51 | NKG5 | NKG5 protein | | |
| M16750_s_at | M16750_s_at | M16750 | PASS | 9 | 34.89 | PASS | 13 | 9 | 13.92 | 2.51 | 2.51 | PIM1 | 6p21 | pim-1 oncogene | pim-1 oncogene |
| K120_NP25 | D21261_at | D21261 | PASS | 9 | 278.78 | PASS | 13 | 9 | 111.31 | 2.50 | 2.50 | TAGLN2 | 1121-q25 | transgelin 2 | transgelin 2 |
| PRKACG | U42412_at | U42412 | PASS | 8 | 16.38 | PASS | 11 | 8 | 6.55 | 2.50 | 2.50 | PRKAG1 | 12q12-q14 | protein kinase, AMP- | protein kinase, AMP- |
| | | | | | | | | | | | | | | activated, gamma 1 non- | activated, gamma 1 non- |
| | | | | | | | | | | | | | | catalytic subunit | catalytic subunit |
| U41315_rna1 | U41315_rna1 | U41315 | PASS | 9 | 15.00 | PASS | 11 | 9 | 6.00 | 2.50 | 2.50 | ZNF127-Xp | | ZNF127-Xp | ring zing-finger protein; |
| | | | | | | | | | | | | | | | escapes X chromosome |
| | | | | | | | | | | | | | | | inactivation |
| NF116 | HG3494-HT3 | HG3494-HT | PASS | 9 | 80.56 | PASS | 13 | 9 | 32.23 | 2.50 | 2.50 | | | | |
| ANX11 | L19605_at | L19605 | PASS | 9 | 97.56 | PASS | 13 | 9 | 39.08 | 2.50 | 2.50 | ANX11 | 10q22-q23 | annexin XI (56 kD | annexin XI (56 kD |
| | | | | | | | | | | | | | | autoantigen) | autoantigen) |
| K25 | D14695_at | D14695 | PASS | 8 | 17.88 | PASS | 12 | 8 | 7.17 | 2.49 | 2.49 | KIAA0025 | | | KIAA0025 gene product |
| K144_DAGK | D63478_at | D63478 | PASS | 7 | 13.43 | PASS | 13 | 7 | 5.38 | 2.49 | 2.49 | KIAA0144 | | | KIAA0144 gene product |
| S100A6 | HG2788-HT2 | HG2788-HT | PASS | 9 | 179.22 | PASS | 13 | 9 | 72.15 | 2.48 | 2.48 | | | | |
| PUTDNABP | U49278_at | U49278 | PASS | 8 | 27.63 | PASS | 13 | 8 | 11.15 | 2.48 | 2.48 | UBE2V2 | | ubiquitin-conjugating | ubiquitin-conjugating |
| | | | | | | | | | | | | | | enzyme E2 variant 2 | enzyme E2 variant 2 |
| HG3395-HT3 | HG3395-HT3 | HG3395-HT | PASS | 7 | 12.71 | PASS | 7 | 7 | 5.14 | 2.47 | 2.47 | | | | |
| BCL6 | U00115_at | U00115 | PASS | 7 | 13.71 | PASS | 9 | 7 | 5.56 | 2.47 | 2.47 | BCL6 | 3q27 | B-cell CLL/lymphoma 6 | B-cell CLL/lymphoma 6 |
| | | | | | | | | | | | | | | (zinc finger protein 51) | (zinc finger protein 51) |
| SAFB | L43631_at | L43631 | PASS | 9 | 25.44 | PASS | 13 | 9 | 10.31 | 2.47 | 2.47 | SAFB | 19p13 | scaffold attachment | scaffold attachment |
| | | | | | | | | | | | | | | factor B | factor B |
| SRFGLYCP | Z50022_at | Z50022 | PASS | 8 | 32.00 | PASS | 13 | 8 | 13.00 | 2.46 | 2.46 | C21ORF1 | 21q22.3 | chromosome 21 open | chromosome 21 open |
| | | | | | | | | | | | | | | reading frame 1 | reading frame 1 |
| MSN | M69066_at | M69066 | PASS | 9 | 178.78 | PASS | 13 | 9 | 72.85 | 2.45 | 2.45 | MSN | Xq11 2-q12 | moesin | moesin |
| PPP4C | X70218_at | X70218 | PASS | 7 | 27.43 | PASS | 11 | 7 | 11.18 | 2.45 | 2.45 | PPP4C | 16p12-16p11 | protein phosphatase 4 | protein phosphatase 4 |
| | | | | | | | | | | | | | | (formerly X), catalytic | (formerly X), catalytic |
| | | | | | | | | | | | | | | subunit | subunit |
| EMP3 | U52101_at | U52101 | PASS | 9 | 159.33 | PASS | 13 | 9 | 65.15 | 2.45 | 2.45 | EMP3 | | epithelial membrane | epithelial membrane |
| | | | | | | | | | | | | | | protein 3 | protein 3 |
| TPI1 | HG2279-HT2 | HG2279-HT | PASS | 9 | 73.33 | PASS | 13 | 9 | 30.00 | 2.44 | 2.44 | | | | |
| K121 | D50911_at | D50911 | PASS | 9 | 16.44 | PASS | 11 | 9 | 6.73 | 2.44 | 2.44 | KIAA0121 | | | KIAA0121 gene product |
| M83652_s_at | M83652_s_at | M83652 | PASS | 9 | 46.56 | PASS | 13 | 9 | 19.08 | 2.44 | 2.44 | PFC | Xp11 4 | properdin P factor, | properdin P factor |
| | | | | | | | | | | | | | | complement | complement |
| PLBK | U78095_at | U78095 | PASS | 5 | 26.40 | PASS | 11 | 5 | 10.82 | 2.44 | 2.44 | | | bikunin | member of the Kunitz |
| | | | | | | | | | | | | | | | family of protease |
| | | | | | | | | | | | | | | | inhibitors |
| FKBP1 | M34539_at | M34539 | PASS | 9 | 42.78 | PASS | 13 | 9 | 17.54 | 2.44 | 2.44 | FKBP1A | 20p13 | FK506-binding protein | FK506-binding protein |
| | | | | | | | | | | | | | | 1A (12 kD) | 1A (12 kD) |
| S100A4 | M80563_at | M80563 | PASS | 9 | 213.22 | PASS | 13 | 9 | 87.62 | 243 | 2.43 | S100A4 | 1q12-q22 | S100 calcium-binding | S100 calcium-binding |
| | | | | | | | | | | | | | | protein A4 (calcium | protein A4 (calcium |
| | | | | | | | | | | | | | | protein, calvaculm, | |
| | | | | | | | | | | | | | | metastasin, murine | metastasin, murine |
| | | | | | | | | | | | | | | placental homolog) | placental homolog) |
| UQCRC1 | L16842_at | L16842 | PASS | 9 | 24.11 | PASS | 12 | 9 | 9.92 | 2.43 | 2.43 | UQCRC1 | 3p21 | ubiquinol-cytochrome c | ubiquinol-cytochrome c |
| | | | | | | | | | | | | | | reductase core protein 1 | reductase core protein 1 |
| Y10807_s_at | Y10107_s_at | Y10807 | PASS | 7 | 35.14 | PASS | 13 | 7 | 14.46 | 2.43 | 2.43 | HRMT1L2 | 19q13 | HMT1 (hnRNP methyl- | HMT1 (hnRNP methyl- |
| | | | | | | | | | | | | | | transferase, S. cerevisiae)- | transferase, S. cerevisiae)- |
| | | | | | | | | | | | | | | like 2 | like 2 |
| SELP | M25322_at | M25322 | PASS | 9 | 15.00 | PASS | 11 | 9 | 6.18 | 2.43 | 2.43 | SELP | 1q22-q25 | selectin P (granule | selectin P (granule |
| | | | | | | | | | | | | | | membrane protein 140 kD, | membrane protein 140 kD, |
| | | | | | | | | | | | | | | antigen CD62) | antigen CD62) |
| PTGS1 | M59979_at | M59979 | PASS | 8 | 15.25 | PASS | 7 | 8 | 6.29 | 2.43 | 2.43 | PTGS1 | 9q32—q33.3 | prostaglandin endoperoxide | prostaglandin-endoperoxide |
| | | | | | | | | | | | | | | synthase | synthase 1 (prostaglandin |
| | | | | | | | | | | | | | | | G/H synthase and cyclo- |
| | | | | | | | | | | | | | | | oxygenase) |
| PIL | U46751_at | U46751 | PASS | 9 | 99.78 | PASS | 13 | 9 | 40.77 | 2.42 | 2.42 | P62 | | UBIQUITIN-BINDING | UBIQUITIN-BINDING |
| | | | | | | | | | | | | | | PROTEIN P62, | PROTEIN P62, |
| | | | | | | | | | | | | | | phosphotyrosine | phosphotyrosine |
| | | | | | | | | | | | | | | independent ligand for the | independent ligand for the |
| | | | | | | | | | | | | | | Lck SH2 domain p62 | Lck SH2 domain p62 |
| ITPK1 | U51336_at | Y51336 | PASS | 9 | 49.00 | PASS | 13 | 9 | 20.23 | 2.42 | 2.42 | | | inositol 1,3,4-tris- | |
| | | | | | | | | | | | | | | phosphate 5/6-kinase | |
| KNS2 | L04733_at | L04733 | PASS | 7 | 17.29 | PASS | 7 | 7 | 7.14 | 242 | 2.42 | | | kinesin light chain | putative |
| M23323_s_at | M23323_s_at | M23323 | PASS | 9 | 45.78 | PASS | 13 | 9 | 18.92 | 2.42 | 2.42 | CD3E | 11q23 | CD3E antigen, epsilon | CD3E antigen, epsilon |
| | | | | | | | | | | | | | | polypeptide (TiT3 complex) | polypeptide (TiT3 complex) |
| X76223_s_at | X76223_at | X76223 | PASS | 7 | 36.86 | PASS | 12 | 7 | 15.25 | 2.42 | 2.42 | MAL | 2cen-q13 | mal, T-cell differentiation | mal, T-cell differentiation |
| | | | | | | | | | | | | | | protein | protein |
| OS9 | U41635_at | U41635 | PASS | 9 | 58.56 | PASS | 13 | 9 | 24.23 | 2.42 | 2.42 | | | OS-9 precursor | ubiquitously expressed in |
| | | | | | | | | | | | | | | | human tissues and |
| | | | | | | | | | | | | | | | amplified in sarcoma |
| RPS6KA2 | L07597_at | L07597 | PASS | 9 | 28.78 | PASS | 12 | 9 | 11.92 | 2.41 | 2.41 | RPS6KA1 | 3 | ribosomal protein S6 | ribosomal protein S6 |
| | | | | | | | | | | | | | | kinase, 90 kD, polypeptide | kinase, 90 kD, polypeptide |
| | | | | | | | | | | | | | | 1 | 1 |
| IFNG | L07633_at | L07633 | PASS | 9 | 84.33 | PASS | 13 | 9 | 34.92 | 2.41 | 2.41 | PSME1 | 14q11.2 | interferon-gamma | proteasome (prosome, |
| | | | | | | | | | | | | | | | macropain) activator |
| | | | | | | | | | | | | | | | subunit 1 (PA28 alpha) |
| FRAPI. | L37033_at | L37033 | PASS | 8 | 29.38 | PASS | 11 | 8 | 12.18 | 2.41 | 2.41 | FKBP38 | | FK-506 binding protein | |
| | | | | | | | | | | | | | | homologue | |
| CES1 | L07765_at | L07765 | PASS | 7 | 15.43 | PASS | 10 | 7 | 6.40 | 2.41 | 2.41 | CES1 | 16q13-q22.1 | carboxylesterase 1 | carboxylesterase 1 |
| | | | | | | | | | | | | | | (monocyte/macrophage | monocyte/macrophage |
| | | | | | | | | | | | | | | serine esterase 1) | serine esterase 1) |
| X56681_s_at | X56681_s_at | X56681 | PASS | 9 | 114.44 | PASS | 13 | 9 | 47.54 | 2.41 | 2.41 | JUND | 19p13.2 | junD protein | jun D proto-oncogene |
| HDLBP | M64088_at | M64098 | PASS | 8 | 20.00 | PASS | 13 | 8 | 8.31 | 2.41 | 2.41 | HBP | | high density lipoprotein | |
| | | | | | | | | | | | | | | binding protein | |
| ECGF1_rna3 | U62317_rna3 | U62317 | PASS | 9 | 66.22 | PASS | 13 | 9 | 27.54 | 2.40 | 2.40 | | | arylsulfatase A | hypothetical protein |
| | | | | | | | | | | | | | | | 384D8 2 |
| K140 | D50930_at | D50930 | PASS | 8 | 18.13 | PASS | 11 | 8 | 7.55 | 2.40 | 2.40 | KIAA0140 | | | |
| HG4541-HT4 | HG4541-HT4 | HG4541-HT | PASS | 9 | 41.56 | PASS | 13 | 9 | 17.31 | 240 | 2.40 | | | | |
| ARP | M83751_at | M83751 | PASS | 9 | 21.22 | PASS | 13 | 9 | 8.85 | 2.40 | 2.40 | ARP | | arginine-rich protein | putative |
| HG417-HT41 | HG417-HT41 | HG417-HT | PASS | 9 | 71.33 | PASS | 13 | 9 | 29.77 | 2.40 | 2.40 | | | | |
| STM | U20499_at | U20499 | PASS | 9 | 20.33 | PASS | 12 | 9 | 8.50 | 239 | 2.39 | SULT1A3 | 16p112 | thermolabile phenol | sulfotransferase family |
| | | | | | | | | | | | | | | sulfotransferase | 1A, phenol-preferring, |
| | | | | | | | | | | | | | | | member 3 |
| NP | K02574_at | K02574 | PASS | 8 | 32.88 | PASS | 13 | 8 | 13.77 | 2.39 | 2.39 | NP | 14111.2 | nucleoside phosphorylase | nucleoside phosphorylase |
| GLA | X14448_at | X14448 | PASS | 9 | 20.56 | PASS | 13 | 9 | 8.62 | 2.39 | 2.39 | | | alpha D-galactosidase A | |
| ARNP | M74002_at | M74002 | PASS | 9 | 20.00 | PASS | 13 | 9 | 8.38 | 2.39 | 2.39 | SFRS11 | 1p21-p34 | splicing factor, arginine/ | splicing factor, arginine/ |
| | | | | | | | | | | | | | | serine-rich 11 | serine-rich 11 |
| K168 | D79990_at | D79990 | PASS | 9 | 2933 | PASS | 13 | 9 | 12.31 | 2.38 | 2.38 | KIAA0168 | | | KIAA0168 gene product |
| SUPT4H1 | U43923_at | U43923 | PASS | 8 | 21.63 | PASS | 12 | 8 | 9.08 | 2.38 | 2.38 | SUPT4H1 | 17q21-q23 | suppressor of Ty (S. | suppressor of Ty (S. |
| | | | | | | | | | | | | | | cerevisiae) 4 homolog 1 | cerevisiae) 4 homolog 1 |
| K174 | D79996_at | D79996 | PASS | 9 | 28.56 | PASS | 13 | 9 | 12.00 | 2.38 | 2.38 | K1AA0174 | | | KIAA0174 gene product |
| DCT | U49785_at | U49785 | PASS | 9 | 24.89 | PASS | 13 | 9 | 10.46 | 2.38 | 2.38 | DDT | 22q11.2 | D-dopachrome tautomerase | D-dopachrome tautomerase |
| CLP36 | U90878_at | U90878 | PASS | 9 | 26.56 | PASS | 12 | 9 | 11.17 | 2.38 | 2.38 | CLIM1 | 10q22-q27 | carboxyl terminal LIM | carboxy terminal LIM |
| | | | | | | | | | | | | | | domain protein | domain protein 1 |
| LAMP5 | U51240_at | U51240 | PASS | 9 | 146.89 | PASS | 13 | 9 | 61.77 | 2.38 | 2.38 | LAPTm5 | | lysosomal-associated | |
| | | | | | | | | | | | | | | multitransmembrane protein | |
| NK4 | M59807_at | M59807 | PASS | 9 | 116.67 | PASS | 13 | 9 | 49.15 | 2.37 | 2.37 | NK4 | 16p13.3 | natural killer cell | natural killer cell |
| | | | | | | | | | | | | | | transcript 4 | transcript 4 |
| K223_COSZ1 | D86976_at | D86976 | PASS | 9 | 103.11 | PASS | 13 | 9 | 43.46 | 2.37 | 2.37 | KIAA0223 | | | similar to C. elegans |
| | | | | | | | | | | | | | | | protein (Z37093) |
| B94 | M92357_at | M92357 | PASS | 9 | 27.00 | PASS | 13 | 9 | 11.38 | 237 | 2.37 | | | B94 protein | |
| SPARC | J303040_at | J03040 | PASS | 9 | 57.78 | PASS | 13 | 9 | 24.38 | 2.37 | 2.37 | SPARC | 5q31-q33 | secreted protein, acidic | secreted protein, acidic |
| | | | | | | | | | | | | | | cysteine-rich (osteonectin) | cystein-rich (osteonectin) |
| PPGB | M22960_at | M22960 | PASS | 9 | 83.44 | PASS | 13 | 9 | 35.23 | 2.37 | 2.37 | PPGB | 20q13.1 | protective protein for | protective protein for |
| | | | | | | | | | | | | | | beta-galactosidase | beta-galactosidase |
| | | | | | | | | | | | | | | | (galactosialidosis) |
| MX2 | M30818_at | M30818 | PASS | 9 | 20.56 | PASS | 13 | 9 | 8.69 | 2.36 | 2.36 | MX2 | 21q22.3 | interferon-induced Mx | myxovirus (influenza) |
| | | | | | | | | | | | | | | protein | resistance 2, homolog |
| | | | | | | | | | | | | | | | of murine |
| SMRT | U37146_at | U37146 | PASS | 9 | 26.56 | PASS | 13 | 9 | 11.23 | 2.36 | 2.36 | SMRT | | silencing mediator of | transcriptional co-repressor |
| | | | | | | | | | | | | | | retinoid and thyroid | |
| | | | | | | | | | | | | | | hormone action | |
| DGK5Z | U51477_at | U51477 | PASS | 9 | 32.56 | PASS | 13 | 9 | 13.77 | 2.36 | 2.36 | DGKZ | | diacylglycerol kinase, | diacylglycerol kinase, |
| | | | | | | | | | | | | | | zeta (104 kD) | zeta (104 kD) |
| LAMP1 | J04182_at | J04182 | PASS | 9 | 47.78 | PASS | 13 | 9 | 20.23 | 2.36 | 2.36 | LAMP1 | | lysosomal membrane | precursor |
| | | | | | | | | | | | | | | glycoprotein-1 | |
| YWHAE | U54778_at | U54778 | PASS | 8 | 14.50 | PASS | 13 | 8 | 6.15 | 2.36 | 2.36 | | | 14-3-3 epsilon | |
| U51333_s_at | U51333_s_at | U51333 | PASS | 9 | 47.11 | PASS | 13 | 9 | 20.00 | 2.36 | 2.36 | HK3 | 5g35.2 | hexokinase 3 (white cell) | hexokinase 3 (white cell) |
| CRFB4 | Z17227_at | Z17227 | PASS | 9 | 15.78 | PASS | 10 | 9 | 6.70 | 2.35 | 2.35 | IL10RB | 21g22.1-g22.1 | interleukin 10 receptor, | interleukin 10 receptor, |
| | | | | | | | | | | | | | | beta | beta |
| PIM2 | U77735_at | U77735 | PASS | 6 | 24.33 | PASS | 12 | 6 | 10.33 | 2.35 | 2.35 | | | pim-2 protooncogene | similar to murine pim-2 |
| | | | | | | | | | | | | | | homolog pim-2h | product endoded by |
| | | | | | | | | | | | | | | | GenBank Accession |
| | | | | | | | | | | | | | | | Number L41495; serine/ |
| | | | | | | | | | | | | | | | threonine protein kinase |
| AAMP | M95627_at | M95627 | PASS | 9 | 22.11 | PASS | 12 | 9 | 9.42 | 2.35 | 2.35 | AAMP | | angio-associated, migratory | angio-associated, migratory |
| | | | | | | | | | | | | | | cell protein | cell protein |
| K67_TOP2 | D31891_at | D31891 | PASS | 9 | 18.56 | PASS | 11 | 9 | 7.91 | 2.35 | 2.35 | KIAA0067 | | | KIAA0067 gene product |
| NKG2D | X54870_at | X54870 | PASS | 9 | 31.33 | PASS | 13 | 9 | 13.38 | 2.34 | 2.34 | NKG2-D | | | type II integral membrane |
| | | | | | | | | | | | | gene | | | protein |
| M81695_s_at | M81695_s_at | M81695 | PASS | 9 | 32.22 | PASS | 13 | 9 | 13.77 | 2.34 | 2.34 | ITGAX | 16p13.1-p11 | integrin, alpha X (antigen | integrin, alpha X (antigen |
| | | | | | | | | | | | | | | CD11C (p150), alpha | CD11C (p150), alpha |
| | | | | | | | | | | | | | | polypeptide) | polypeptide) |
| KRT12 | U77643_at | U77643 | PASS | 8 | 28.25 | PASS | 13 | 8 | 12.08 | 2.34 | 2.34 | SECTM1 | 17g25 | secreted and trans- | secreted and trans- |
| | | | | | | | | | | | | | | membrane 1 | membrane 1 |
| LGALS9 | AB006782_at | AB006782 | PASS | 9 | 78.89 | PASS | 13 | 9 | 33.77 | 2.34 | 2.34 | LGALS9 | | lectin, galactoside-binding, | lectin, galactoside-binding, |
| | | | | | | | | | | | | | | soluble, 9 (galectin 9) | soluble 9 (galectin 9) |
| ARF3 | M74491_at | M74491 | PASS | 9 | 54.11 | PASS | 13 | 9 | 23.23 | 2.33 | 2.33 | ARP3 | 12q13 | ADP-ribosylation factor 3 | ADP-ribosylation factor 3 |
| ALDH7 | U10868_at | U10868 | PASS | 8 | 16.88 | PASS | 12 | 8 | 7.25 | 2.33 | 2.33 | ALDH7 | 11g13 | aldehyde dehydrogenase 7 | aldehyde dehydrogenase 7 |
| M54915_s_at | M54915_s_at | M54915 | PASS | 9 | 54.67 | PASS | 13 | 9 | 23.54 | 2.32 | 2.32 | | | pim-1 protein | |
| FAH | M55150_at | M55150 | PASS | 6 | 18.50 | PASS | 8 | 6 | 8.00 | 2.31 | 2.31 | FAH | 15q23-q25 | furnarylacetoacetate | furnarylacetoacetate |
| TPM3 | HG3514-HT3 | HG3514-HT | PASS | 9 | 149.22 | PASS | 13 | 9 | 64.54 | 2.31 | 2.31 | | | | |
| CAKB | U43522_at | U43522 | PASS | 8 | 14.13 | PASS | 9 | 8 | 6.11 | 2.31 | 2.31 | PTK2B | 8p21.1 | focal adhesion kinase | protein tyrosine kinase |
| | | | | | | | | | | | | | | 2 (protein kinase B) | 2 beta |
| ICAM3 | X69819_at | X69819 | PASS | 9 | 52.22 | PASS | 13 | 9 | 22.62 | 2.31 | 2.31 | ICAM3 | 19p13.3-p13.2 | intercellular adhesion | intercellular adhesion |
| | | | | | | | | | | | | | | molecule 3 | molecule 3 |
| IRF5 | U51127_at | U51127 | PASS | 9 | 29.00 | PASS | 7 | 9 | 12.57 | 2.31 | 2.31 | IRFS | 7q32 | interferon regulatory | interferon regulatory |
| | | | | | | | | | | | | | | molecule 3 | molecule 3 |
| CAP | L12168_at | L12168 | PASS | 9 | 134.67 | PASS | 13 | 9 | 58.38 | 2.31 | 2.31 | CAP | | adenylyl cyclase- | putative |
| | | | | | | | | | | | | | | associated protein | |
| RBPS6 | U51334_at | U51334 | PASS | 8 | 25.50 | PASS | 13 | 8 | 11.08 | 2.30 | 2.30 | TAF2N | 17q11.1-q11.2 | TATA box binding protein | TATA box binding protein |
| | | | | | | | | | | | | | | (TBP)-associated factor, | (TBP)-associated factor, |
| | | | | | | | | | | | | | | RNA polymerase II, | RNA polymerase II, |
| | | | | | | | | | | | | | | N, 68 kD (RNA-binding | N, 68 kD (RNA-binding |
| | | | | | | | | | | | | | | protein 56) | protein 56) |
| HSPAIL_rna | M11717_rna | M11717 | PASS | 9 | 53.11 | PASS | 13 | 9 | 2308 | 2.30 | 2.30 | HSPAIL | | heat shock protein | 70 kDa |
| RGL2 | U68142_at | U68142 | PASS | 9 | 15.67 | PASS | 11 | 9 | 6.82 | 2.30 | 2.30 | RGL2 | | RalGDS-like | |
| PM5 | X57398_at | X57398 | PASS | 9 | 27.33 | PASS | 12 | 9 | 11.92 | 2.29 | 2.29 | pM5 | | pm5 protein | Protein sequence is in |
| | | | | | | | | | | | | | | | conflict with the |
| | | | | | | | | | | | | | | | conceptual translation |
| K217 | D86971_at | D86971 | PASS | 5 | 17.20 | PASS | 10 | 5 | 7.50 | 2.29 | 2.29 | KIAA0217 | | | no similarities to |
| | | | | | | | | | | | | | | | reported gene products |
| CAPG | M94345_at | M94345 | PASS | 9 | 49.33 | PASS | 13 | 9 | 21.54 | 2.29 | 2.29 | CAPG | 2cen-q24 | capping protein (actin | capping protein (actin |
| | | | | | | | | | | | | | | filament), gelsolin-like | filament), gelsolin-like |
| PIN1 | U49070_at | U49070 | PASS | 6 | 14.50 | PASS | 12 | 6 | 6.33 | 2.29 | 2.29 | PIN1 | | Pin1 | NIMA-interacting protein |
| | | | | | | | | | | | | | | | 1, essential mitotic |
| | | | | | | | | | | | | | | | regulator, essential |
| | | | | | | | | | | | | | | | peptidyl-prolyl isomerase |
| U72882_s_at | U72882_s_at | U72882 | PASS | 6 | 15.50 | PASS | 9 | 6 | 6.78 | 2.29 | 229 | IFP35 | | interferon-induced leucine | |
| | | | | | | | | | | | | | | zipper protein | |
| ITGAM | J03925_at | J03925 | PASS | 9 | 23.56 | PASS | 13 | 9 | 10.31 | 2.29 | 2.29 | ITGAM | 16p11.2 | integrin, alpha M | integrin, alpha M |
| | | | | | | | | | | | | | | (complement component | (complement component |
| | | | | | | | | | | | | | | receptor 3, alpha; | receptor 3; alpha; |
| | | | | | | | | | | | | | | alpha; also known as | alpha; also known as |
| | | | | | | | | | | | | | | CD11b (p170), macrophage | CD11b (p170), macrophage |
| | | | | | | | | | | | | | | antigen alpha polypeptide) | antigen alpha polpeptide) |
| IQGAP2 | U51903_at | U51903 | PASS | 8 | 17.38 | PASS | 13 | 8 | 7.62 | 2.28 | 2.28 | IQGAP2 | | RasGAP-related protein | IQGAP2; Cdc42-, Rac1-, |
| | | | | | | | | | | | | | | | and calmodulin-binding |
| | | | | | | | | | | | | | | | protein |
| MLN62 | X80200_at | X80200 | PASS | 9 | 15.11 | PASS | 8 | 9 | 6.63 | 2.28 | 2.28 | TRAF4 | 17q11-q12 | TNF receptor-associated | TNF receptor-associated |
| | | | | | | | | | | | | | | factor 4 | factor 4 |
| INPP5D | U57650_at | U57650 | PASS | 9 | 42.78 | PASS | 13 | 9 | 18.77 | 2.28 | 2.28 | INPP5D | 2q36-q37 | SH2-containing inositol 5- | inositol polyphosphate- |
| | | | | | | | | | | | | | | phosphatase | 5-phosphatase, 145 kD |
| M13829_s_at | M13829_s_at | M13829 | PASS | 8 | 15.25 | PASS | 13 | 8 | 6.69 | 2.28 | 2.28 | ARAF1 | Xp11.4-p11.2 | V-raf murine sarcoma | v-raf murine sarcoma |
| | | | | | | | | | | | | | | 3611 viral oncogene | 3611 viral oncogene |
| | | | | | | | | | | | | | | homolog 1 | homolog 1 |
| ITGB2 | M15395_at | M15395 | PASS | 9 | 86.11 | PASS | 13 | 9 | 37.85 | 2.28 | 2.28 | ITGB2 | 21q22.3 | integrin, beta 2 (antigen | integrin, beta 2 (antigen |
| | | | | | | | | | | | | | | CD18 (p95), lymphocyte | CD18 (p95), lymphocyte |
| | | | | | | | | | | | | | | function-associated | function-associated |
| | | | | | | | | | | | | | | antigen 1; macrophage | antigen 1; macrophage |
| | | | | | | | | | | | | | | antigen 1 (mac-1) beta | antigen 1 (mac-1) beta |
| | | | | | | | | | | | | | | subunit) | subunit) |
| D43682_s_at | D43682_s_at | D43682 | PASS | 9 | 41.44 | PASS | 13 | 9 | 18.23 | 2.27 | 2.27 | ACADVL | 17p13-p11 | acyl-Coenzyme A | acyl-Coenzyme A |
| | | | | | | | | | | | | | | dehydrogenase, very | dehydrogenase, very |
| | | | | | | | | | | | | | | long chain | long chain |
| FTH1 | L20941_at | L20941 | PASS | 9 | 279.00 | PASS | 13 | 9 | 122.77 | 2.27 | 2.27 | FTH1 | 11q13 | ferritin, heavy polypeptide | ferritin, heavy polypeptide |
| | | | | | | | | | | | | | | 1 | 1 |
| PSMHC9 | D00763_at | D00763 | PASS | 9 | 42.78 | PASS | 12 | 9 | 18.83 | 2.27 | 2.27 | PSMA4 | | proteasome (prosome, | proteasome (prosome, |
| | | | | | | | | | | | | | | macropain) subunit, alpha | macropain) subunit, alpha |
| | | | | | | | | | | | | | | type, 4 | type, 4 |
| AKT1 | M63167_at | M63167 | PASS | 8 | 23.13 | PASS | 11 | 8 | 10.18 | 2.27 | 2.27 | AKT1 | 14q32.3 | rac protein kinase-alpha | v-akt murine thymorna viral |
| | | | | | | | | | | | | | | | oncogene homolog 1 |
| POGA | L24783_at | L24783 | PASS | 7 | 14.29 | PASS | 10 | 7 | 6.30 | 2.27 | 2.27 | | | | |
| K106_B15C | D14662_at | D14662 | PASS | 9 | 36.44 | PASS | 13 | 9 | 16.08 | 2.27 | 2.27 | KIAA0106 | 1 | anti-oxidant protein 2 | anti-oxidant protein 2 |
| | | | | | | | | | | | | | | (non-selenium glutathione | (non-selenium glutathione |
| | | | | | | | | | | | | | | peroxidase, acidic calcium- | peroxidase, acidic calcium- |
| | | | | | | | | | | | | | | independent phospholipase | independent phospholipase |
| | | | | | | | | | | | | | | A2 | A2 |
| CYP2A6_f | X13930_f_at | X13930 | PASS | 5 | 13.60 | PASS | 9 | 5 | 6.00 | 2.27 | 2.27 | | | | P-450 IIA4 protein |
| | | | | | | | | | | | | | | | (AA 1-494) |
| K113 | D30755_at | D30755 | PASS | 9 | 29.11 | PASS | 13 | 9 | 12.85 | 2.27 | 2.27 | KIAA0113 | | | |
| P115RHOGE | U64105_at | U64105 | PASS | 9 | 43.56 | PASS | 13 | 9 | 19.23 | 2.26 | 2.26 | SUB1.5 | 19q13.13 | guanine nucleotide | guanine nucleotide |
| | | | | | | | | | | | | | | exchange factor; 115- kD; | exchange factor; 115- kD; |
| | | | | | | | | | | | | | | mouse Lsc homolog | mouse Lsc homolog |
| TALDO1 | L19437_at | L19437 | PASS | 9 | 85.33 | PASS | 13 | 9 | 37.69 | 2.26 | 2.26 | | | transaldolase | |
| PSMD2 | D78151_at | D78151 | PASS | 9 | 36.22 | PASS | 13 | 9 | 16.00 | 2.26 | 2.26 | PSMD2 | | proteasome (prosome, | proteasome (prosome, |
| | | | | | | | | | | | | | | macropain) 26S subunit, | macropain) 26S subunit, |
| | | | | | | | | | | | | | | non-ATPase, 2 | non-ATPase, 2 |
| LGALS1 | J04456_at | J04456 | PASS | 9 | 102.89 | PASS | 13 | 9 | 45.46 | 2.26 | 2.26 | LGALS1 | 22q12-q13 | lectin, galactoside-binding, | lectin, galactoside-binding |
| | | | | | | | | | | | | | | soluble, 1 (galectin 1) | soluble, 1 (galectin 1) |
| UFD1L | U64444_at | U64444 | PASS | 9 | 22.78 | PASS | 13 | 9 | 10.08 | 2.26 | 2.26 | UFD1L | | ubiquitin fusion- | ubiquitin like protein |
| | | | | | | | | | | | | | | degradation 1 like | |
| | | | | | | | | | | | | | | protein | |
| K68 | D38549_at | D38549 | PASS | 8 | 19.63 | PASS | 10 | 8 | 8.70 | 226 | 2.26 | KIAA0068 | | | |
| PBX1 | M86546_at | M86546 | PASS | 6 | 12.50 | PASS | 11 | 6 | 5.55 | 2.25 | 2.25 | PBX1 | 1q23 | pre-B-cell leukemia | pre-B-cell leukemia |
| | | | | | | | | | | | | | | transcription factor 1 | transcription factor 1 |
| RAC2 | HG1102-HT1 | HG1102-HT | PASS | 9 | 20.11 | PASS | 13 | 9 | 8.92 | 2.25 | 2.25 | | | | |
| PRKMK2 | L11285_at | L11285 | PASS | 9 | 29.11 | PASS | 13 | 9 | 12.92 | 2.25 | 2.25 | PRKMK2 | | | protein kinase, mitogen- |
| | | | | | | | | | | | | | | | activated, kinase 2, p45 |
| | | | | | | | | | | | | | | | (MAP kinase kinase 2) |
| K82_ACNPV | D43949_at | D43949 | PASS | 8 | 16.75 | PASS | 11 | 8 | 7.45 | 2.25 | 2.25 | KIAA0082 | | | This gene is novel |
| PACE4 | M80482_at | M80482 | PASS | 7 | 11.71 | PASS | 9 | 7 | 5.22 | 2.24 | 2.24 | PACE4 | 15q26 | paired basic amino acid | paired basic amino acid |
| | | | | | | | | | | | | | | cleaving system 4 | cleaving system 4 |
| GMCSFIND | S69115_at | S69115 | PASS | 8 | 80.38 | PASS | 13 | 8 | 35.85 | 2.24 | 2.24 | granulocyte | | | This sequence comes from |
| | | | | | | | | | | | | colony- | | | FIG. 3. |
| | | | | | | | | | | | | stimulating | | | |
| | | | | | | | | | | | | factor | | | |
| | | | | | | | | | | | | induced gene | | | |
| ZAP70 | L05148_at | L05148 | PASS | 9 | 36.56 | PASS | 13 | 9 | 16.31 | 2.24 | 2.24 | | | | |
| X96506_s_at | X96506_s_at | X96506 | PASS | 5 | 22.40 | PASS | 10 | 5 | 10.00 | 2.24 | 2.24 | NC2 | | | alpha subunit; forms |
| | | | | | | | | | | | | | | | heterodimer with NC2 |
| | | | | | | | | | | | | | | | alpha/Dr1 |
| BRCA2 | U50535_at | U50535 | PASS | 9 | 15.67 | PASS | 12 | 9 | 7.00 | 2.24 | 2.24 | | | | |
| | | | | | | | | | | | | | | subunit | |
| PPP2R1A | J02902_at | J02902 | PASS | 8 | 33.38 | PASS | 12 | 8 | 14.92 | 2.24 | 2.24 | | | phosphatase 2A regulatory | |
| | | | | | | | | | | | | | | subunit | |
| IL2RB | M26062_at | M26062 | PASS | 8 | 35.25 | PASS | 13 | 8 | 15.77 | 2.24 | 2.24 | IL2RB | 22q13 | interleukin 2 receptor, | interleukin 2 receptor, |
| | | | | | | | | | | | | | | beta | beta |
| DJ1 | D61380_at | D61380 | PASS | 9 | 58.44 | PASS | 13 | 9 | 26.15 | 2.23 | 2.23 | | | DJ-1 protein | |
| UBL1 | D23662_at | D23662 | PASS | 9 | 53.11 | PASS | 13 | 9 | 23.77 | 2.23 | 2.23 | | | ubiquitin-like protein | |
| ZNF173 | U09825_at | U09825 | PASS | 9 | 21.44 | PASS | 13 | 9 | 9.62 | 2.23 | 2.23 | ZNF173 | 6p21.3 | zinc finger protein 173 | |
| UCP2 | U94592_at | U94592 | PASS | 9 | 50.22 | PASS | 13 | 9 | 22.54 | 2.23 | 2.23 | UCPH | | uncoupling protein | |
| | | | | | | | | | | | | | | homolog | |
| L35249_s_at | L35249_s_at | L35249 | PASS | 9 | 33.89 | PASS | 13 | 9 | 15.23 | 2.23 | 2.23 | ATP6B2 | | ATPase, H+ transporting, | ATPase, H+ transporting, |
| | | | | | | | | | | | | | | lysosomal (vacuolar | lysosomal (vacuolar |
| | | | | | | | | | | | | | | proton pump), beta | proton pump), beta |
| | | | | | | | | | | | | | | polypeptide, 56/58 kD, | polypeptide, 56/58 kD, |
| | | | | | | | | | | | | | | isoform 2 | isoform 2 |
| SLA | D89077_at | D89077 | PASS | 9 | 31.89 | PASS | 12 | 9 | 14.33 | 2.22 | 2.22 | | | Src-like adapter protein | |
| TUBB2 | HG1980-HT2 | HG1980-HT | PASS | 9 | 53.22 | PASS | 13 | 9 | 23.92 | 2.22 | 2.22 | | | | |
| K88 | D42041_at | D42041 | PASS | 9 | 21.56 | PASS | 13 | 9 | 9.69 | 2.22 | 2.22 | KIAA0088 | | | The ha1225 gene product |
| | | | | | | | | | | | | | | | is related to human |
| | | | | | | | | | | | | | | | alpha-glucosidase. |
| GUSB | M15182_at | M15182 | PASS | 9 | 18.89 | PASS | 12 | 9 | 8.50 | 2.22 | 2.22 | GUSB | 7q22 | glucuronidase, beta | glucuronidase, beta |
| RAD23A | D21235_at | D21235 | PASS | 9 | 15.56 | PASS | 10 | 9 | 7.00 | 2.22 | 2.22 | RAD23A | 19p13.2 | HHR23A protein | RAD23 (S. cerevisiae) |
| | | | | | | | | | | | | | | | homolog A |
| TRAIL | U37518_at | U37518 | PASS | 9 | 3444 | PASS | 13 | 9 | 15.54 | 2.22 | 2.22 | TNPSF10 | 3q26 | tumor necrosis factor | tumor necrosis factor |
| | | | | | | | | | | | | | | (ligand) superfamily, | (ligand) superfamily, |
| | | | | | | | | | | | | | | member 10 | member 10 |
| UNP | U20657_at | U20657 | PASS | 9 | 14.78 | PASS | 12 | 9 | 6.67 | 2.22 | 2.22 | USP4 | 13p21.3 | ubiquitin specific protease, | ubiquitin specific protease |
| | | | | | | | | | | | | | | proto-oncogene | 4 (proto-oncogene) |
| PDIRP5 | D49489_at | D49489 | PASS | 9 | 19.22 | PASS | 13 | 9 | 8.69 | 2.21 | 2.21 | | | human P5 | The transcript is amplified |
| | | | | | | | | | | | | | | | in hydroxyurea-resistant |
| | | | | | | | | | | | | | | | cells; an endoplasmic |
| | | | | | | | | | | | | | | | reticulum-retention signal |
| | | | | | | | | | | | | | | | (ER-retention signal) at |
| | | | | | | | | | | | | | | | 1403-1414; two |
| | | | | | | | | | | | | | | | thioredoxin-like sequences |
| | | | | | | | | | | | | | | | (Trx-like motifs) at |
| | | | | | | | | | | | | | | | 254-271, 659-676 |
| OGDH | D10523_at | D10523 | PASS | 7 | 13.57 | PASS | 7 | 7 | 6.14 | 2.21 | 2.21 | OGDH | 7p13-p11.2 | oxoglutarate dehydrogenase | oxoglutarate dehydrogenase |
| | | | | | | | | | | | | | | (lipoamide) | (lipoamide) |
| PALMPTH | U44772_at | U44772 | PASS | 9 | 26.00 | PASS | 13 | 9 | 11.77 | 2.21 | 2.21 | PPT | 1p32 | palmitoyl-protein | palmitoyl-protein |
| | | | | | | | | | | | | | | thioesterase (ceroid- | thioesterase (ceroid- |
| | | | | | | | | | | | | | | lipfuscinosis, neuronal 1, | lipfuscinosis, neuronal 1, |
| | | | | | | | | | | | | | | infantile; Haltia- | infantile; Haltia- |
| | | | | | | | | | | | | | | Santavuori disease) | Santavuori disease) |
| ATPLP | D89052_at | D89052 | PASS | 9 | 53 11 | PASS | 13 | 9 | 24.08 | 2.21 | 2.21 | ATP6F | 1p32.3 | ATPase, H+ transporting, | ATPase, H+ transporting, |
| | | | | | | | | | | | | | | lysosomal (vacuolar proton | lysosomal (vacuolar proton |
| | | | | | | | | | | | | | | pump) 21 kD | pump) 21 kD |
| FGR | M19722_at | M19722 | PASS | 9 | 94.78 | PASS | 13 | 9 | 43.00 | 2.20 | 220 | PGR | 1p362-p36.1 | | Gardner-Rasheed feline |
| | | | | | | | | | | | | | | | sarcoma viral (v-fgr) |
| | | | | | | | | | | | | | | | oncogene homolog |
| PCMT1 | D25547_at | D25547 | PASS | 9 | 13.22 | PASS | 9 | 9 | 6.00 | 2.20 | 2.20 | | | PIMT isozyme I | |
| NCF2 | M32011_at | M32011 | PASS | 9 | 53.22 | PASS | 13 | 9 | 24.23 | 2.20 | 2.20 | NCF2 | 1cen-q32 | neutrophil cyttosolic factor | neutrophil cytosolic factor |
| | | | | | | | | | | | | | | 2 (65 kD) | 2 (65 kD, chronic |
| | | | | | | | | | | | | | | | granulomatous disease, |
| | | | | | | | | | | | | | | | autosomal 2) |
| HG998-HT99 | HG998-HT99 | HG998-HT | PASS | 9 | 21.00 | PASS | 12 | 9 | 9.58 | 2.19 | 2.19 | | | | |
| K218_HYP29 | D86972_at | D86972 | PASS | 9 | 13.78 | PASS | 10 | 9 | 6.30 | 2.19 | 2.19 | KIAA0218 | | | KIAA0218 gene product |
| H2A2 | L19779_at | L19779 | PASS | 9 | 71.89 | PASS | 13 | 9 | 32.92 | 2.18 | 2.18 | H2AFO | | H2A histone family, | H2A histone family, |
| | | | | | | | | | | | | | | member O | member O |
| Z47038_s_at | Z47038 s_at | Z47038 | PASS | 8 | 12.88 | PASS | 11 | 8 | 5.91 | 2.18 | 2.18 | putative | | | open reading frame; |
| | | | | | | | | | | | | microtabule | | | N-terminal region |
| | | | | | | | | | | | | associated | | | |
| | | | | | | | | | | | | protein 1A | | | |
| K224_DDX | D86977_at | D86977 | PASS | 8 | 15.25 | PASS | 13 | 8 | 7.00 | 2.18 | 2.18 | KIAA0224 | | | KIAA0224 gene product |
| K160 | D63881_at | D63881 | PASS | 9 | 14.56 | PASS | 13 | 9 | 6.69 | 2.17 | 2.17 | KIAA0160 | | | KIAA0160 gene product |
| | | | | | | | | | | | | | | | is novel. |
| D83260_s_at | D83260_s_at | D83260 | PASS | 9 | 16.56 | PASS | 13 | 9 | 762 | 2.17 | 2.17 | DXS9928E | Xq28 | putative candidate disease | putative candidate disease |
| | | | | | | | | | | | | | | gene XAP5 | gene XAP5 |
| EIF3 | U78525_at | U78525 | PASS | 9 | 19.56 | PASS | 13 | 9 | 9.00 | 2.17 | 2.17 | EIF3S9 | | eukaryotic translation | eukaryotic translation |
| | | | | | | | | | | | | | | initiation factor 3, | initiation factor 3, |
| | | | | | | | | | | | | | | subunit 9 (eta, 116 kD) | subunit 9 (eta, 116 kD) |
| K169 | D79991_at | D79991 | PASS | 9 | 13.67 | PASS | 10 | 9 | 6.30 | 2.17 | 2.17 | KIAA0169 | | | putative hydrophobic |
| | | | | | | | | | | | | | | | domain in amino acid |
| | | | | | | | | | | | | | | | positions 373-390. |
| GZMA_rna1 | M18737_rna1 | M18737 | PASS | 9 | 75.22 | PASS | 13 | 9 | 34.69 | 2.17 | 2.17 | GZMA | 5q21-q12 | | granzyme A (granzyme 1, |
| | | | | | | | | | | | | | | | cytotoxic T-lmphocyte- |
| | | | | | | | | | | | | | | | associated serine |
| | | | | | | | | | | | | | | | esterase 3) |
| PP1 | U14603_at | U14603 | PASS | 9 | 76.56 | PASS | 13 | 9 | 35.31 | 2.17 | 2.17 | PTP4A2 | 1p35 | protein tyrosine | protein tyrosine |
| | | | | | | | | | | | | | | phosphatase type IVA, | phosphatase type IVA, |
| | | | | | | | | | | | | | | member 2 | member 2 |
| MLF2 | U57342_at | U57342 | PASS | 9 | 26.00 | PASS | 13 | 9 | 12.00 | 2.17 | 2.17 | MLF2 | | myelodysplasia/myeloid | |
| | | | | | | | | | | | | | | leukemia factor 2 | |
| M84371_rna1 | M84371_rna1 | M84371 | PASS | 8 | 14.38 | PASS | 11 | 8 | 6.64 | 2.17 | 2.17 | CD19 | | | |
| H1X | D64142_at | D64142 | PASS | 9 | 54.89 | PASS | 13 | 9 | 25.38 | 2.16 | 2.16 | H1FX | | histone H1x | H1 histone family, |
| | | | | | | | | | | | | | | | member X |
| CMKBR2_rn | U95626_rna1 | U95626 | PASS | 8 | 36.75 | PASS | 13 | 8 | 17.00 | 2.16 | 2.16 | ccr2 | | ccr2a | confirmed by similarity |
| | | | | | | | | | | | | | | | to Human monocyte |
| | | | | | | | | | | | | | | | chemoattractant protein |
| | | | | | | | | | | | | | | | 1 receptor (ccr2) |
| | | | | | | | | | | | | | | | alternatively spliced |
| | | | | | | | | | | | | | | | A-form, Encoded by |
| | | | | | | | | | | | | | | | GenBank Accession |
| | | | | | | | | | | | | | | | Number U80924, |
| | | | | | | | | | | | | | | | gi 1168965 |
| U32986_s_at | U32986_s_at | U32986 | PASS | 9 | 24.67 | PASS | 12 | 9 | 11.42 | 2.16 | 2.16 | DDB1 | 11q12-q13 | damage-specific DNA | damage-specific DNA |
| | | | | | | | | | | | | | | binding protein 1 (127 kD) | binding protein 1 (127 kD) |
| MPP1 | M64925_at | M64925 | PASS | 9 | 34.89 | PASS | 12 | 9 | 16.17 | 2.16 | 2.16 | MPP1 | Xq28 | membrane protein, | membrane protein, |
| | | | | | | | | | | | | | | palmitoylated 1 (55 kD) | palmitoylated 1 (55 kD) |
| BCL2 | M14745_at | M14745 | PASS | 9 | 16.22 | PASS | 13 | 9 | 7.54 | 2.15 | 2.15 | | | | |
| DAGK1 | X62535_at | X62535 | PASS | 9 | 38.56 | PASS | 13 | 9 | 17.92 | 2.15 | 2.15 | DGKA | 12 | diacylglycerol kinase | diacylglycerol kinase, |
| | | | | | | | | | | | | | | | alpha (80 kD) |
| M63438_s_at | M63438_s_at | M63438 | PASS | 9 | 167.11 | PASS | 13 | 9 | 77.77 | 2.15 | 2.15 | | | | |
| TRADD | L41690_at | L41690 | PASS | 9 | 18.00 | PASS | 13 | 9 | 8.38 | 2.15 | 2.15 | TRADD | | tumor necrosis factor | TNFRSF1A-associated |
| | | | | | | | | | | | | | | receptor type 1 associated | via death domain |
| | | | | | | | | | | | | | | protein | |
| PGM1 | M83088_at | M83088 | PASS | 9 | 16.67 | PASS | 13 | 9 | 7.77 | 2.15 | 2.15 | PGM1 | 1p22.1 | phosphoglucomutase 1 | phosphoglucomutase 1 |
| CRIP1 | U09770_at | U09770 | PASS | 9 | 35.33 | PASS | 12 | 9 | 16.50 | 2.14 | 2.14 | hCRHP | | cysteine-rich heart protein | |
| K43_HOM | D26362_at | D26362 | PASS | 8 | 17.13 | PASS | 9 | 8 | 8.00 | 2.14 | 2.14 | KIAA0043 | | | KIAA0043 gene product |
| MYD88 | U70451_at | U70451 | PASS | 8 | 38.13 | PASS | 13 | 8 | 17.85 | 2.14 | 2.14 | MYD88 | 3p22 | myeloid differentiation | myeloid differentiation |
| | | | | | | | | | | | | | | primary response gene (88) | primary response gene (88) |
| HNRPH1 | L22009_at | L22009 | PASS | 9 | 74.00 | PASS | 13 | 9 | 34.69 | 2.13 | 2.13 | | | hnRNP H | 49 kDa protein; hetero- |
| | | | | | | | | | | | | | | | geneous nuclear ribo- |
| | | | | | | | | | | | | | | nucleoprotein H | |
| MXI1 | L07648_at | L07648 | PASS | 9 | 17.56 | PASS | 13 | 9 | 8.23 | 2.13 | 2.13 | MXI1 | | | |
| GUK1 | L76200_at | L76200 | PASS | 8 | 56.25 | PASS | 13 | 8 | 26.38 | 2.13 | 2.13 | GUK1 | 1q32-q42 | guanylate kinase 1 | guanylate kinase 1 |
| C8FWPH | AJ000480_at | AJ000480 | PASS | 5 | 11.60 | PASS | 9 | 5 | 5.44 | 2.13 | 2.13 | C8FW | | phosphoprotein | |
| GNG11 | U31384_at | U31384 | PASS | 9 | 38.78 | PASS | 13 | 9 | 18.23 | 2.13 | 2.13 | GNG11 | | guanine nucleotide binding | guanine nucleotide binding |
| | | | | | | | | | | | | | | protein 11 | protein 11 |
| HG3076-HT3 | HG3076-HT3 | HG3076-HT | PASS | 9 | 52.33 | PASS | 13 | 9 | 24.62 | 2.13 | 2.13 | | | | |
| UGT2B4 | U03105_at | U03105 | PASS | 7 | 19.86 | PASS | 11 | 7 | 9.36 | 2.12 | 2.12 | | | B4-2 protein | |
| DPYSL2 | U97105_at | U97105 | PASS | 8 | 18.75 | PASS | 13 | 8 | 8.85 | 2.12 | 2.12 | DPYSL2 | 8p22-p21 | dihydropyrimidinase-like 2 | dihydropyrimidinase-like 2 |
| GGTB2 | D29805_at | D29805 | PASS | 9 | 40.89 | PASS | 13 | 9 | 19.31 | 2.12 | 2.12 | B4GALT1 | 9p13 | glycoprotein-4-beta- | UDP-Gal:betaGlcNAc beta |
| | | | | | | | | | | | | | | galactosyltransferase 2 | 1,4-galactosyltransferase, |
| | | | | | | | | | | | | | | | polypeptide 1 |
| M61827_rna1 | M61827_rna1 | M61827 | PASS | 9 | 33.22 | PASS | 10 | 9 | 15.70 | 2.12 | 2.12 | SPN | | leukosialin | |
| D63479_s_at | D63479_s_at | D63479 | PASS | 9 | 18.67 | PASS | 12 | 9 | 8.83 | 2.11 | 2.11 | DGKD | | diacylglycerol kinase, | diacylglycerol kinase, |
| | | | | | | | | | | | | | | delta (130 kD) | delta (130 kD) |
| IP | L47738_at | L47738 | PASS | 9 | 29.22 | PASS | 12 | 9 | 13.83 | 2.11 | 2.11 | | | | inducible protein |
| X98534_s_at | X98534_s_at | X98534 | PASS | 8 | 25.50 | PASS | 13 | 8 | 12.08 | 2.11 | 2.11 | VASP | 19q13.2-q13.3 | vasodilator-stimulated | vasodilator-stimulated |
| | | | | | | | | | | | | | | phosphoprotein | phosphoprotein |
| CALM1 | HG1862-HT1 | HG1862-HT | PASS | 9 | 91.11 | PASS | 13 | 9 | 43.15 | 2.11 | 2.11 | | | | |
| HPK1 | U66464_at | U66464 | PASS | 9 | 16.89 | PASS | 13 | 9 | 8.00 | 2.11 | 2.11 | HPK1 | | hematopoietic progenitor | serine/threonine protein |
| | | | | | | | | | | | | | | | kinase |
| FKBP2 | M75099_at | M75099 | PASS | 9 | 23.56 | PASS | 12 | 9 | 11.17 | 2.11 | 2.11 | FKBP2 | 11q13.1-q13.3 | FK506-binding protein 2 | FK506-binding protein 2 |
| | | | | | | | | | | | | | | (13 kD) | (13 kD) |
| CMKBR7 | L31584_at | L31584 | PASS | 9 | 39.89 | PASS | 13 | 9 | 18.92 | 2.11 | 2.11 | CCR7 | 17q12-q21.2 | chemokine (C—C motif) | chemokine (C—C motif) |
| | | | | | | | | | | | | | | receptor 7 | receptor 7 |
| GPRK6 | L16862_at | L16862 | PASS | 7 | 25.29 | PASS | 7 | 7 | 12.00 | 2.11 | 2.11 | GPRK6 | 5q35 | G protein-coupled | G protein-coupled |
| | | | | | | | | | | | | | | receptor kinase 6 | receptor kinase 6 |
| FCER1G | M33195_at | M33195 | PASS | 9 | 112.00 | PASS | 13 | 9 | 53.15 | 2.11 | 2.11 | FCER1G | 1q23 | Fc fragment of IgE, | Fc fragment of IgE, |
| | | | | | | | | | | | | | | high affinity I, | high affinity I, |
| | | | | | | | | | | | | | | receptor for; gamma | receptor for; gamma |
| | | | | | | | | | | | | | | polypeptide | polypeptide |
| MRP | HG1612-HT1 | HG1612-HT | PASS | 8 | 20.00 | PASS | 12 | 8 | 9.50 | 2.11 | 2.11 | | | | |
| LYL1 | M22638_at | M22638 | PASS | 8 | 14.38 | PASS | 12 | 8 | 6.83 | 2.10 | 2.10 | LYL1 | | | |
| IRAK1 | L76191_at | L76191 | PASS | 9 | 32.67 | PASS | 13 | 9 | 15.54 | 2.10 | 2.10 | IRAKI | Xq28 | interleukin-1 receptor- | interleukin-1 receptor- |
| | | | | | | | | | | | | | | associated kinase 1 | associated kinase 1 |
| P14KB | U81802_at | U81802 | PASS | 7 | 14.71 | PASS | 11 | 7 | 7.00 | 2.10 | 2.10 | PIK4CB | 1q21 | phosphatidylmositol 4- | phosphatidylmositol 4- |
| | | | | | | | | | | | | | | kinase, catalytic, beta | kinase, catalytic, beta |
| | | | | | | | | | | | | | | polypeptide | polypeptide |
| GT335 | U53003_at | U53003 | PASS | 9 | 12.22 | PASS | 11 | 9 | 5.82 | 2.10 | 2.10 | GT335 | | | similar to E. coli SCRP27A |
| | | | | | | | | | | | | | | | and to zebrafish ES1 |
| M58286_s_at | M58286_s_at | M58286 | PASS | 7 | 17.43 | PASS | 10 | 7 | 8.30 | 2.10 | 2.10 | TNFRSF1A | 12p13.2 | tumor necrosis factor | tumor necrosis factor |
| | | | | | | | | | | | | | | receptor 1 (55 kD) | receptor superfamily, |
| | | | | | | | | | | | | | | | member 1A |
| PSMA28 | D45248_at | D45248 | PASS | 9 | 69.44 | PASS | 13 | 9 | 33.08 | 2.10 | 2.10 | PSME2 | 14q11.2 | proteasome (prosome, | proteasome (prosome, |
| | | | | | | | | | | | | | | macropain) activator | macropain) activator |
| | | | | | | | | | | | | | | subunit 2 (PA28 beta) | subunit 2 (PA28 beta) |
| CKAP1 | D49738_at | D49738 | PASS | 9 | 34.56 | PASS | 13 | 9 | 16.46 | 2.10 | 2.10 | CKAP1 | 19q13.11- | cytoskeleton-associated | cytoskeleton-associated |
| | | | | | | | | | | | | | q13.12 | protein 1 | protein 1 |
| HG4334-HT4 | HG4334-HT4 | HG4334-HT | PASS | 6 | 14.50 | PASS | 12 | 6 | 6.92 | 2.10 | 2.10 | | | | |
| SRF | J03161_at | J03161 | PASS | 6 | 18.83 | PASS | 9 | 6 | 9.00 | 2.09 | 2.09 | SRF | | serum response factor | serum response factor |
| | | | | | | | | | | | | | | (c-fos serum response | (c-fos serum response |
| | | | | | | | | | | | | | | element-binding | element-binding |
| | | | | | | | | | | | | | | transcription factor) | transcription factor) |
| CRAA | U78556_at | U78556 | PASS | 8 | 15.75 | PASS | 13 | 8 | 7.54 | 2.09 | 2.09 | hCRA alpha | | cisplatin resistance | |
| | | | | | | | | | | | | | | associated alpha protein | |
| S83513_s_at | S83513_s_at | S83513 | PASS | 7 | 11.14 | PASS | 9 | 7 | 5.33 | 2.09 | 2.09 | ADCYAP1 | 18p11 | adenylate cyclase activating | adenylate cyclase activating |
| | | | | | | | | | | | | | | polypeptide 1 (primary) | polypeptide 1 (primary) |
| VCL | M33308_at | M33308 | PASS | 9 | 50.22 | PASS | 13 | 9 | 24.08 | 2.09 | 2.09 | VCL | 10q11.2-qter | vinculin | vinculin |
| K183 | D80005_at | D80005 | PASS | 9 | 25.00 | PASS | 13 | 9 | 12.00 | 2.08 | 2.08 | KIAA0183 | | | |
| J04046_s_at | J04046_s_at | J04046 | PASS | 8 | 29.13 | PASS | 12 | 8 | 14.00 | 2.08 | 2.08 | CALM1 | | | calmodulin |
| STXBP3 | D63851_at | D63851 | PASS | 5 | 9.80 | PASS | 7 | 5 | 4.71 | 2.08 | 2.08 | STXBP1 | 9q34.1 | syntaxin binding protein 1 | syntaxin-binding protein 1 |
| COPA | U24105_at | U24105 | PASS | 9 | 36.78 | PASS | 13 | 9 | 17.69 | 2.08 | 208 | COPA | | coatomer protein complex, | coatomer protein complex, |
| | | | | | | | | | | | | | | subunit alpha | subunit alpha |
| DDB2 | U18300_at | U18300 | PASS | 9 | 12.11 | PASS | 12 | 9 | 5.83 | 2.08 | 2.08 | DDB2 | 11p12-p11 | damage-specific DNA | damage-specific DNA |
| | | | | | | | | | | | | | | binding protein 2 (48 kD) | binding protein 2 (48 kD) |
| MLN_rna1 | X15393_rna1 | X15393 | PASS | 7 | 13.29 | PASS | 10 | 7 | 6.40 | 2.08 | 2.08 | motilin | | motinlin | |
| GAPDH3 | AFFX-HUM | AFFX-HUM | PASS | 9 | 318.33 | PASS | 13 | 9 | 153.54 | 2.07 | 2.07 | | | | |
| P2RX5 | U49395_at | U49395 | PASS | 9 | 17.44 | PASS | 12 | 9 | 8.42 | 2.07 | 2.07 | P2RX5 | | purinergic receptor P2X, | purinergic receptor P2X, |
| | | | | | | | | | | | | | | ligand-gated ion channel, 5 | ligand-gated ion channel, 5 |
| CDC42 | U02570_at | U02570 | PASS | 9 | 25.56 | PASS | 12 | 9 | 12.33 | 2.07 | 2.07 | ARHGAP1 | | Rho GTPase activating | Rho GTPase activating |
| | | | | | | | | | | | | | | protein 1 | protein 1 |
| L10338_s_at | L10338_s_at | L10338 | PASS | 7 | 12.43 | PASS | 8 | 7 | 6.00 | 2.07 | 2.07 | SCN1B | 19 | sodium channel, voltage- | sodium channel, voltage- |
| | | | | | | | | | | | | | | gated, type 1, beta | gated, type 1, beta |
| | | | | | | | | | | | | | | polypeptide | polypeptide |
| A1P | U29680_at | U29680 | PASS | 8 | 15.00 | PASS | 12 | 8 | 7.25 | 2.07 | 2.07 | BCL2A1 | 15q24.3 | BCL2-related protein A1 | BCL2-related protein A1 |
| Z69043_s_at | Z69043_s_at | Z69043 | PASS | 9 | 57.11 | PASS | 13 | 9 | 27.62 | 2.07 | 2.07 | H-TRAP | | translocon-associated | |
| | | | | | | | | | | | | delta | | protein delta subunit | |
| | | | | | | | | | | | | | | precursor | |
| HAX1 | U68566_at | U68566 | PASS | 9 | 31.00 | PASS | 11 | 9 | 15.00 | 2.07 | 2.07 | | | HAX-1 | localized to the mito- |
| | | | | | | | | | | | | | | | chondrial membrane, HS1 |
| | | | | | | | | | | | | | | | binding protein |
| CREB2 | D90209_at | D90209 | PASS | 9 | 57.89 | PASS | 13 | 9 | 28.08 | 2.06 | 2.06 | ATF4 | | activating transcription | activating transcription |
| | | | | | | | | | | | | | | factor 4 (tax-responsive | factor 4 (tax-responsive |
| | | | | | | | | | | | | | | enhancer | enhancer element B67) |
| ZFP_r | HG3565-HT3 | HG3565-HT | PASS | 8 | 35.50 | PASS | 9 | 8 | 17.22 | 2.06 | 2.06 | | | | |
| RH18019 | U24166 at | U24166 | PASS | 9 | 28.22 | PASS | 13 | 9 | 13.69 | 2.06 | 2.06 | | | EB1 | |
| STAT4 | L78440_at | L78440 | PASS | 9 | 22.33 | PASS | 13 | 9 | 10.85 | 2.06 | 2.06 | STAT4 | 2q32.2-q32.3 | signal transducer and | signal transducer and |
| | | | | | | | | | | | | | | activator of | activator of |
| | | | | | | | | | | | | | | transcription 4 | transcription 4 |
| COX6B_rna2 | AC002115_rn | AC002115 | PASS | 8 | 17.50 | PASS | 10 | 8 | 850 | 2.06 | 2.06 | COX6B | | F25451_2 | hypothetical 36.5 kDa |
| | | | | | | | | | | | | | | | protein most similar to |
| | | | | | | | | | | | | | | | ssRNA binding proteins, |
| | | | | | | | | | | | | | | | BLASTX similarity to |
| | | | | | | | | | | | | | | | (Y07952) ssRNA-binding |
| | | | | | | | | | | | | | | | protein [Dictyostelium |
| | | | | | | | | | | | | | | | discoideum] (52%) |
| | | | | | | | | | | | | | | | within RNP domains; and |
| | | | | | | | | | | | | | | | to (Z70043) hypothetical |
| | | | | | | | | | | | | | | | 24.4 kD protein C22E12.02 |
| | | | | | | | | | | | | | | | in chromosome I |
| | | | | | | | | | | | | | | | [Schizosaccharomyces |
| | | | | | | | | | | | | | | | pombe] |
| HK1 | M75126_at | M75126 | PASS | 9 | 58.56 | PASS | 13 | 9 | 28.46 | 2.06 | 2.06 | HK1 | 10q22 | hexokinase 1 | hexokinase 1 |
| HH109 | D23673_at | D23673 | PASS | 9 | 32.11 | PASS | 13 | 9 | 15.62 | 2.06 | 2.06 | | | | |
| RPL39 | D79205_at | D79205 | PASS | 9 | 465.11 | PASS | 13 | 9 | 226.31 | 2.06 | 2.06 | RPL39 | | ribosomal protein L39 | ribosomal protein L39 |
| M92843_a_at | M92843_s_at | M92843 | PASS | 9 | 33.33 | PASS | 13 | 9 | 16.23 | 2.05 | 2.05 | ZFP36 | 19q13.1 | zinc finger protein | zinc finger protein |
| | | | | | | | | | | | | | | homologous to Zfp-36 | homologous to Zfp-36 |
| | | | | | | | | | | | | | | in mouse | in mouse |
| P19INK4D | U40343_at | U40343 | PASS | 9 | 27.44 | PASS | 13 | 9 | 13.38 | 2.05 | 2.05 | CDKN2D | 19p13 | cyclin-dependent kinase | cyclin-dependent kinase |
| | | | | | | | | | | | | | | inhibitor 2D (p19, inhibits | inhibitor 2D (p19, inhibits |
| | | | | | | | | | | | | | | CDK4) | CDK4) |
| PFN1 | J03191_at | J03191 | PASS | 9 | 184.22 | PASS | 13 | 9 | 90.00 | 2.05 | 2.05 | PFN1 | 17p13.3 | | profilin 1 |
| TFDP1 | M73547_at | M73547 | PASS | 9 | 22.67 | PASS | 13 | 9 | 11.08 | 2.05 | 2.05 | DP1 | | polyposis locus-encoded | |
| | | | | | | | | | | | | | | protein | |
| RAB32 | U59878_at | U59878 | PASS | 6 | 16.17 | PASS | 11 | 6 | 7.91 | 2.04 | 2.04 | RAB32 | | low-Mr GTP-binding | Low-Mr GTP-binding |
| | | | | | | | | | | | | | | protein Rab32 | protein of the Rab |
| | | | | | | | | | | | | | | | subfamily |
| X97267_rna1 | X97267_rna1 | X97267 | PASS | 9 | 75.00 | PASS | 13 | 9 | 36.69 | 2.04 | 2.04 | | | LPAP | |
| QARS | X76013_at | X76013 | PASS | 9 | 37.22 | PASS | 13 | 9 | 18.23 | 2.04 | 2.04 | QARS | 3 | glutaminyl-tRNA | glutamine-tRNA |
| | | | | | | | | | | | | | | synthetase | synthetase |
| NFAT4C | L41067_at | L41067 | PASS | 9 | 16.33 | PASS | 13 | 9 | 8.00 | 2.04 | 2.04 | NFATC4 | | nuclear factor of | nuclear factor of |
| | | | | | | | | | | | | | | activated T-cells, | activated T-cells, |
| | | | | | | | | | | | | | | cytoplasmic 4 | cytoplasmic 4 |
| PF4 | M25897_at | M25897 | PASS | 9 | 278.33 | PASS | 13 | 9 | 136.46 | 2.04 | 2.04 | PF4V1 | 4q12-q21 | platelet factor 4 | platelet factor 4 |
| | | | | | | | | | | | | | | variant 1 | variant 1 |
| RPA2 | J05249_at | J05249 | PASS | 9 | 30.11 | PASS | 13 | 9 | 14.77 | 2.04 | 2.04 | RPA2 | 1 | replication protein A2 | replication protein A2 |
| | | | | | | | | | | | | | | (32 kD) | (32 kD) |
| CNN1 | D83735_at | D83735 | PASS | 9 | 66.00 | PASS | 13 | 9 | 32.38 | 2.04 | 2.04 | CNN2 | 21q11.1 | calponin 2 | calponin 2 |
| AFPX-HSAC | APFX-HSAC | AFFX-HSA | PASS | 9 | 153.44 | PASS | 13 | 9 | 75.31 | 2.04 | 2.04 | | | | |
| 100KDCOA | U22055_at | U22055 | PASS | 9 | 39.33 | PASS | 13 | 9 | 19.31 | 2.04 | 2.04 | | | 100 kDa coactivator | |
| ETR3 | U69546_at | U69546 | PASS | 7 | 22.71 | PASS | 13 | 7 | 11.15 | 2.04 | 2.04 | | | Etr-3 | protein contains 3 RRM |
| | | | | | | | | | | | | | | | motifs that may bind RNA; |
| | | | | | | | | | | | | | | | putative RNA binding |
| | | | | | | | | | | | | | | | protein; Elav-type ribo- |
| | | | | | | | | | | | | | | | nucleoprotein; complete |
| | | | | | | | | | | | | | | | sequence of human EST, |
| | | | | | | | | | | | | | | | GenBank Accession |
| | | | | | | | | | | | | | | | Number R57293 |
| GSK3A | L40027_at | L40027 | PASS | 9 | 19.89 | PASS | 13 | 9 | 9.77 | 2.04 | 2.04 | | | glycogen synthase kinase 3 | |
| HLA-A_f | D32129_f_at | D32129 | PASS | 9 | 342.11 | PASS | 13 | 9 | 168.23 | 2.03 | 2.03 | | | HLA-A26 | exon 1 part (leader region) |
| | | | | | | | | | | | | | | | bp6-77; exon 2 part (alpha- |
| | | | | | | | | | | | | | | | 1 domain) bp78-347; exon |
| | | | | | | | | | | | | | | | 3 part (alpha-2 domain); |
| | | | | | | | | | | | | | | | bp348-623; exon 4 part |
| | | | | | | | | | | | | | | | (alpha-3 domain: bp624- |
| | | | | | | | | | | | | | | | 899; exon 5 part (trans- |
| | | | | | | | | | | | | | | | membrane region). bp900- |
| | | | | | | | | | | | | | | | 1016; exon 6 part (cyto- |
| | | | | | | | | | | | | | | | plasmic region): bp 1050- |
| | | | | | | | | | | | | | | | 1097; exon 8 part (cyto- |
| | | | | | | | | | | | | | | | plasmic region): bp1098- |
| | | | | | | | | | | | | | | | 1103 |
| RBI | L22343_at | L22343 | PASS | 7 | 12.86 | PASS | 9 | 7 | 6.33 | 2.03 | 2.03 | | | nuclear phosphoprotein | IFN-induced |
| PIK4 | L36151_at | L36151 | PASS | 9 | 39.89 | PASS | 13 | 9 | 19.69 | 2.03 | 2.03 | PIK4CA | | phosphatidylinositol 4- | phosphatidylinositol 4- |
| | | | | | | | | | | | | | | kinase, catalytic, alpha | kinase, catalytic, alpha |
| | | | | | | | | | | | | | | polypeptide | polypeptide |
| SPS2 | U43286_at | U43286 | PASS | 9 | 13.67 | PASS | 12 | 9 | 6.75 | 2.02 | 2.02 | SP52 | | selenophosphate synthetase | the amino acid residue |
| | | | | | | | | | | | | | | 2 | number 60 is a seleno- |
| | | | | | | | | | | | | | | | cysteine encoded by the |
| | | | | | | | | | | | | | | | TGA (UGA) codon; We |
| | | | | | | | | | | | | | | | designated this enzyme |
| | | | | | | | | | | | | | | | selenophosphate synthetase |
| | | | | | | | | | | | | | | | 2 to distinguish it from the |
| | | | | | | | | | | | | | | | human homolog described |
| | | | | | | | | | | | | | | | by Low, S. C., Harney, |
| | | | | | | | | | | | | | | | J. W. and Berry, M. J. |
| | | | | | | | | | | | | | | | J. Biol Chem. 270, 21659- |
| | | | | | | | | | | | | | | | 21664 (1995) (GenBank |
| | | | | | | | | | | | | | | | Accession Number |
| | | | | | | | | | | | | | | | U34044) |
| HYPA | U81556 at | U81556 | PASS | 9 | 40.44 | PASS | 13 | 9 | 20.00 | 2.00 | 2.02 | | | hypothetical protein A4 | |
| 3PK | U09578_at | U09578 | PASS | 8 | 17.25 | PASS | 13 | 8 | 8.54 | 2.02 | 2.02 | MAPKAPK3 | 3p21.13 | mitogen-activated protein | mitogen-activated protein |
| | | | | | | | | | | | | | | kinase-activated protein | kinase-activated protein |
| | | | | | | | | | | | | | | kinase 3 | kinase 3 |
| K23_CAN | D14689_at | D14689 | PASS | 9 | 32.11 | PASS | 12 | 9 | 15.92 | 2.02 | 2.02 | KIAA0023 | | | KIAA0023 gene product |
| RNA_OSTB | AB000115_at | AB000115 | PASS | 7 | 12.86 | PASS | 8 | 7 | 6.38 | 2.02 | 2.02 | | | | |
| IL2RG | D11086_at | D11086 | PASS | 9 | 42.00 | PASS | 13 | 9 | 20.85 | 2.01 | 2.01 | IL2RG | Xq13 | interleukin 2 receptor, | interleukin 2 receptor, |
| | | | | | | | | | | | | | | gamma (severe combined | gamma (severe combined |
| | | | | | | | | | | | | | | immunodeficiency) | immunodeficiency) |
| SEC14L | D67029_at | D67029 | PASS | 5 | 15.60 | PASS | 12 | 5 | 7.75 | 2.01 | 2.01 | SEC14L | 17q25.1-g25.2 | SEC14 (S. cerevisiae)-like | SEC14 (S. cerevisiae)-like |
| POLG | U60325_at | U60325 | PASS | 6 | 13.33 | PASS | 8 | 6 | 6.63 | 2.01 | 2.01 | POLG | 15q24 | polymerase (DNA | polymerase (DNA |
| | | | | | | | | | | | | | | directed), gamma | directed), gamma |
| K142_K6 | D63476_at | D63476 | PASS | 9 | 14.22 | PASS | 13 | 9 | 7.08 | 2.01 | 2.01 | P85SPR | 13 | PAK-interacting exchange | PAK-interacting exchange |
| | | | | | | | | | | | | | | factor beta | factor beta |
| FCGR1A | M63835_at | M63835 | PASS | 7 | 12.29 | PASS | 8 | 7 | 6.13 | 2.01 | 2.01 | FCGR1A | 1q21.2-q21.3 | IgG Fc receptor 1 | Fc fragment of IgG, |
| | | | | | | | | | | | | | | | high affinity Ia, receptor |
| | | | | | | | | | | | | | | for (CD64) | |
| MLK3 | L32976_at | L32976 | PASS | 7 | 12.29 | PASS | 7 | 7 | 6.14 | 2.00 | 2.00 | MLK3 | 11q13.1-q13.3 | mixed lineage kinase 3 | mixed lineage kinase 3 |
| E_CCA12 | U06681_at | U06681 | PASS | 5 | 12.40 | PASS | 10 | 5 | 6.20 | 2.00 | 2.00 | | | | |
| FACL1 | D10040_at | D10040 | PASS | 5 | 12.00 | PASS | 9 | 5 | 6.00 | 2.00 | 2.00 | FACLI | 3q13 | fatty-acid-Coenzyme A | fatty-acid-Coenzyme A |
| | | | | | | | | | | | | | | ligase, long-chain 1 | ligase, long-chain 1 |
| MCM6 | D84557_at | D84557 | PASS | 9 | 13.44 | PASS | 11 | 9 | 6.73 | 2.00 | 2.00 | | | HsMcm6 | |
| BRCA2 | U50523_at | U50523 | PASS | 9 | 84.89 | PASS | 13 | 9 | 42.54 | 2.00 | 2.00 | | | | |
| BIOC3 | APFX-BioC-1 | AFFX-BioC | fail | 4 | | PASS | 13 | 4 | 14.46 | Normal | Normal | | | | |
| L15189_s_at | L15189_s_at | L15189 | fail | 4 | | PASS | 13 | 4 | 6.77 | Normal | Normal | HSPA9B | 5q31.1 | heat shock 70 kD protein | heat shock 70 kD protein |
| | | | | | | | | | | | | | | 9B (mortalin-2) | 9B (mortalin-2) |
| NIP71 | U83843_at | U83843 | fail | 4 | | PASS | 13 | 4 | 6.77 | Normal | Normal | Nip7-1 | | HIV-1 Nef interacting | similar to murine CCT |
| | | | | | | | | | | | | | | protein | (chaperonin containing |
| | | | | | | | | | | | | | | | TCP-1) cta subunit encoded |
| | | | | | | | | | | | | | | | by GenBank Accession |
| | | | | | | | | | | | | | | | Number Z31399; CCT |
| | | | | | | | | | | | | | | | assists the folding of |
| | | | | | | | | | | | | | | | proteins in eukaryotic |
| | | | | | | | | | | | | | | | cytosol; Nef enhances the |
| | | | | | | | | | | | | | | | infectivity of HIV and SIV |
| APT1 | D49396_at | D49396 | fail | 4 | | PASS | 13 | 4 | 4.85 | Normal | Normal | | | Aop1_Human, MER5 | |
| | | | | | | | | | | | | | | (Aop1_Mouse)-like protein | |
| RAB1 | M28209_at | M28209 | fail | 3 | | PASS | 13 | 3 | 6.23 | Normal | Normal | RAB1 | 2p14-p13.4 | RAB1, member RAS | RAB1, member RAS |
| | | | | | | | | | | | | | | oncogene family | oncogene family |
| M28213_s_at | M28213_s_at | M28213 | fail | 3 | | PASS | 13 | 3 | 5.77 | Normal | Normal | RAB2 | | RAB2, member RAS | RAB2, member RAS |
| | | | | | | | | | | | | | | oncogene family | oncogene family |
| L00634_s_at | L00634_s_at | L00634 | fail | 2 | | PASS | 13 | 2 | 6.15 | Normal | Normal | FNTA | 8q22-q11 | farncsyltransferase, CAAX | farnesyltransferase, CAAX |
| | | | | | | | | | | | | | | box, alpha | box, alpha |
| PEPD | J04605_at | J04605 | fail | 2 | | PASS | 13 | 2 | 5.38 | Normal | Normal | PEPD | 19q12-q13.2 | peptidase D | peptidase D |
| SEC7PL | U59752_at | U59752 | fail | 4 | | PASS | 12 | 4 | 7.92 | Normal | Normal | PSCD2 | | pleckstrin homology, Sec7 | pleckstrin homology, Sec7 |
| PEPD | J04605_at | J04605 | fail | 2 | | PASS | 13 | 2 | 5.38 | Normal | Normal | PEPD | 19q12-q13.2 | | |
| | | | | | | | | | | | | | | and coiled/coil domains | and coiled/coil domains |
| | | | | | | | | | | | | | | 2 (cytohesin-2) | 2 (cytohesin-2) |
| K112 | D25218_at | D25218 | fail | 4 | | PASS | 12 | 4 | 6.42 | Normal | Normal | KIAA0112 | | | |
| PP21 | M99701_at | M99701 | fail | 4 | | PASS | 12 | 4 | 5.75 | Normal | Normal | TCEAL1 | Xq22.1 | transcription elongation | transcription elongation |
| | | | | | | | | | | | | | | factor A (SII)-like 1 | factor A (SII)-like 1 |
| AFFX-BioB-1 | AFFX-BioB-1 | AFFX-BioB | fail | 3 | | PASS | 12 | 3 | 10.75 | Normal | Normal | | | | |
| E_CREBBP | U89355_at | U89355 | fail | 3 | | PASS | 12 | 3 | 5.17 | Normal | Normal | CREBBP | 16p13.3 | CREB binding protein | CREB binding protein |
| | | | | | | | | | | | | | | (Rubinstein-Taybi | (Rubinstein-Taybi |
| | | | | | | | | | | | | | | syndrome) | syndrome) |
| CUL1 | U58087_at | U58087 | fail | 4 | | PASS | 11 | 4 | 8.64 | Normal | Normal | CUL1 | | cullin 1 | cullin 1 |
| L09229_s_at | L09229_s_at | L09229 | fail | 4 | | PASS | 11 | 4 | 8.36 | Normal | Normal | FACL1 | | long-chain acyl-CoA | ATP-binding domain |
| | | | | | | | | | | | | | | synthetase | (bp. 1447 . . . 1846) |
| SLC20A3_rna | X96924_rna1 | X96924 | fail | 4 | | PASS | 11 | 4 | 8.18 | Normal | Normal | | | mitochondrial citrate | |
| | | | | | | | | | | | | | | transport protein | |
| K233_COST1 | C87071_at | D87071 | fail | 4 | | PASS | 11 | 4 | 7.45 | Normal | Normal | KIAA0233 | | | KIAA0233 gene product |
| E_23773 | U90904_at | U90904 | fail | 4 | | PASS | 11 | 4 | 7.00 | Normal | Normal | | | | |
| MTHFD | L38928_at | L38928 | fail | 4 | | PASS | 11 | 4 | 6.73 | Normal | Normal | | | 5,10-methenyltetrahydro- | |
| | | | | | | | | | | | | | | folate synthetase | |
| MEF | U32645_at | U32645 | fail | 4 | | PASS | 11 | 4 | 6.27 | Normal | Normal | ELF4 | | E74-like factor 4 (ets | E74-like factor 4 (ets |
| | | | | | | | | | | | | | | domain transcription factor | domain transcription factor |
| OAT | M29927_at | M29927 | fail | 4 | | PASS | 11 | 4 | 6.00 | Normal | Normal | OAT | 10q26 | ornithine aminotransferase | ornithine aminotransferase |
| | | | | | | | | | | | | | | (gyrate atrophy) | (gyrate atrophy) |
| MPV17 | X76538_at | X76538 | fail | 4 | | PASS | 11 | 4 | 5.82 | Normal | Normal | MPV17 | 2p23-p21 | MpV17 transgene, murine | MpV17 transgene, murine |
| | | | | | | | | | | | | | | homolog, glomerulo- | homolog, glomerulo- |
| | | | | | | | | | | | | | | sclerosis | sclerosis |
| FH | U59309_at | U59309 | fail | 4 | | PASS | 11 | 4 | 5.73 | Normal | Normal | FH | 1q42.1 | fumarate hydratase | fumarate hydratase |
| GBP1 | M55542_at | M55542 | fail | 4 | | PASS | 11 | 4 | 5.64 | Normal | Normal | GBP1 | 1 | guanylate binding protein 1, | guanylate binding protein 1, |
| | | | | | | | | | | | | | | interferon-inducible, 67 kD | interferon-inducible, 67 kD |
| ARF6 | M57763_at | M57763 | fail | 3 | | PASS | 11 | 3 | 7.64 | Normal | Normal | ARF6 | | ADP-ribosylation factor 6 | ADP-ribosylation factor 6 |
| GUBP | U78524_at | U78524 | fail | 3 | | PASS | 11 | 3 | 6.00 | Normal | Normal | DDXBP1 | 15q | DEAD/H (Asp-Glu-Ala- | Dead/H (Asp-Glu-Ala- |
| | | | | | | | | | | | | | | Asp/His) box binding | Asp/His) box binding |
| | | | | | | | | | | | | | | protein 1 | protein 1 |
| E_23828 | U79285_at | U79285 | fail | 3 | | PASS | 11 | 3 | 5.73 | Normal | Normal | | | | |
| PTEN | U92436_at | U92436 | fail | 3 | | PASS | 11 | 3 | 5.00 | Normal | Normal | PTEN | 10q23 | phosphatase and tensin | phosphatase and tensin |
| | | | | | | | | | | | | | | homolog (mutated in | homolog (mutated in |
| | | | | | | | | | | | | | | multiple advanced cancers | multiple advanced cancers |
| | | | | | | | | | | | | | | 1) | 1) |
| SLBP | U75679_at | U75679 | fail | 3 | | PASS | 11 | 3 | 4.91 | Normal | Normal | SLBP | | histone stem-loop binding | |
| | | | | | | | | | | | | | | protein | |
| AFFX-HSAC | AFFX-HSAC | AFFX-HSA | fail | 1 | | PASS | 11 | 1 | 7 | 27 | Normal | Normal | | | |
| U04806_s_at | U04806_s_at | U04806 | fail | 4 | | PASS | 10 | 4 | 8.90 | Normal | Normal | | | FLT3/FLK2 ligand | |
| TRIP7 | L40357_at | L40357 | fail | 4 | | PASS | 10 | 4 | 7.60 | Normal | Normal | TRIP7 | | thyroid receptor interactor | thyroid hormone receptor |
| | | | | | | | | | | | | | | | interactor 7 |
| STAT1 | M97936_at | M97936 | fail | 4 | | PASS | 10 | 4 | 7.50 | Normal | Normal | | | | |
| LRMP | U10485_at | U10485 | fail | 4 | | PASS | 10 | 4 | 7.00 | Normal | Normal | LRMP | | lymphoid-restricted | lymphoid-restricted |
| | | | | | | | | | | | | | | membrane protein | membrane protein |
| LAP18_rna1 | M31303_rna1 | M31303 | fail | 4 | | PASS | 10 | 4 | 6.60 | Normal | Normal | Op18 | | oncoprotein 18 | |
| DIFF48 | U49187_at | U49187 | fail | 4 | | PASS | 10 | 4 | 6.50 | Normal | Normal | Diff48 | | | |
| SNRPD3 | U15009_at | U15009 | fail | 4 | | PASS | 10 | 4 | 6.40 | Normal | Normal | SNRPD3 | | small nuclear ribonucleo- | small nuclear ribonucleo- |
| | | | | | | | | | | | | | | protein D3 polypeptide | protein D3 polypeptide |
| | | | | | | | | | | | | | | (18 kD) | 18 kD) |
| X62153_s_at | X62153_s_at | X62153 | fail | 4 | | PASS | 10 | 4 | 6.30 | Normal | Normal | | | P1.h protein | |
| CTR2 | U83461_at | U83461 | fail | 4 | | PASS | 10 | 4 | 6.20 | Normal | Normal | SLC31A2 | 9q31-q32 | solute carrier family 31 | solute carrier family 31 |
| | | | | | | | | | | | | | | (copper transporters), | (copper transporters), |
| | | | | | | | | | | | | | | member 2 | member 2 |
| GPCR | L42324_at | L42324 | fail | 4 | | PASS | 10 | 4 | 6.10 | Normal | Normal | GPCR | | G protein-linked receptor | |
| EIF5 | U49436_at | U49436 | fail | 4 | | PASS | 10 | 4 | 6.00 | Normal | Normal | EIF5 | | eukaryotic translation | eukaryotic translation |
| | | | | | | | | | | | | | | initiation factor 5 | initiation factor 5 |
| K100_EL | D43947_at | D43947 | fail | 4 | | PASS | 10 | 4 | 5.90 | Normal | Normal | KIAA0100 | | | KIAA0100 gene product |
| K274_HYON | D87464_at | D87464 | fail | 4 | | PASS | 10 | 4 | 5.90 | Normal | Normal | K1AA0274 | | | KIAA0274 gene product |
| GOT2 | M22632_at | M22632 | fail | 4 | | PASS | 10 | 4 | 5.90 | Normal | Normal | GOT2 | 16q21 | glutamic-oxaloacetic | glutamic-oxaloacetic |
| | | | | | | | | | | | | | | transaminase 2, | transaminase 2, |
| | | | | | | | | | | | | | | mitochondrial (aspartate | mitochondrial (aspartate |
| | | | | | | | | | | | | | | aminotransferase 2) | aminotransferase 2) |
| DGUOK | U41668_at | U41668 | fail | 4 | | PASS | 10 | 4 | 5.90 | Normal | Normal | DGUOK | | deoxyguanosien kinase | deoxyguanosine kinase |
| IRRCP | U18321_at | U18321 | fail | 4 | | PASS | 10 | 4 | 5.80 | Normal | Normal | DAP3 | 1q21 | Death associated protein 3 | Death associated protein 3 |
| HZF1 | X78924_at | X78924 | fail | 4 | | PASS | 10 | 4 | 5.80 | Normal | Normal | HZF1 | | zinc finger protein | |
| D29640_s_at | D329640_s_at | D29640 | fail | 4 | | PASS | 10 | 4 | 5.70 | Normal | Normal | SAR1 | 15 | rasGAP-like with IQ motifs | rasGAP-like with IQ motifs |
| SNRPA1 | X13482_at | X13482 | fail | 4 | | PASS | 10 | 4 | 5.60 | Normal | Normal | SNRPA1 | | small nuclear ribonucleo- | small nuclear ribonucleo- |
| | | | | | | | | | | | | | | protein polypeptide A′ | protein polypeptide A′ |
| U47686_s_at | U47686_s_at | U47686 | fail | 4 | | PASS | 10 | 4 | 5.10 | Normal | Normal | | | signal transducer and | STAT protein; is activated |
| | | | | | | | | | | | | | | activator of transcription | by IL-2, IL-7, IL-15, |
| | | | | | | | | | | | | | | Stat5B | growth hormone, IL-3, |
| | | | | | | | | | | | | | | | GM-CSF, thrombopoietin, |
| | | | | | | | | | | | | | | | prolactin, and erythro- |
| | | | | | | | | | | | | | | | poietin; tyrosine 699 |
| | | | | | | | | | | | | | | | phosphorylation is required |
| | | | | | | | | | | | | | | | for activation and dimeriza- |
| | | | | | | | | | | | | | | | tion of Stat5B |
| K267_NAHE | D87743_at | D87743 | fail | 4 | | PASS | 10 | 4 | 4.80 | Normal | Normal | K1AA0267 | | | Similar to Human Na+/H+ |
| | | | | | | | | | | | | | | | exchanger 2 (A57644) |
| RPP38 | U77664_at | U77664 | fail | 4 | | PASS | 10 | 4 | 4.70 | Normal | Normal | RPP38 | | RNaseP protein P38 | |
| L38593_s_at | L38593_s_at | L38593 | fail | 3 | | PASS | 10 | 3 | 6.50 | Normal | Normal | NRAMP1 | | integral membrane protein | alternative |
| TB1 | M74089_at | M74089 | fail | 3 | | PASS | 10 | 3 | 5.50 | Normal | Normal | TB1 | | | |
| CHED | M80629_at | M80629 | fail | 3 | | PASS | 10 | 3 | 5.30 | Normal | Normal | CDC2L | | cholinesterase-related cell | cholinesterase-related cell |
| | | | | | | | | | | | | | | division controller | division controller |
| RCPN | U03644_at | U03644 | fail | 3 | | PASS | 10 | 3 | 5.30 | Normal | Normal | CIR | | CBF1 interacting co- | CBF1 interacting co- |
| | | | | | | | | | | | | | | repressor | repressor |
| CCNG2 | U47414_at | U47414 | fail | 3 | | PASS | 10 | 3 | 5.30 | Normal | Normal | CCNG2 | | cyclin G2 | cyclin G2 |
| E_23721 | U79291_at | U79291 | fail | 3 | | PASS | 10 | 3 | 5.00 | Normal | Normal | | | | |
| LIVP | L13800_at | Ll3800 | fail | 3 | | PASS | 10 | 3 | 4.90 | Normal | Normal | | | | |
| PXMP3 | M86852_at | M86852 | fail | 3 | | PASS | 10 | 3 | 4.80 | Normal | Normal | PXMP3 | 8q21.1 | peroxisomal membrane | peroxisomal membrane |
| | | | | | | | | | | | | | | protein 3 (35 kD, Zellweger | protein 3 (35 kD, Zellweger |
| | | | | | | | | | | | | | | syndrome) | syndrome) |
| CPSPF | U37012_at | U37012 | fail | 4 | | PASS | 9 | 4 | 8.00 | Normal | Normal | | | cleavage and polyadensyla- | 160 kDa subunit |
| | | | | | | | | | | | | | | tion specificity factor | |
| CD9 | M38690_at | M38690 | fail | 4 | | PASS | 9 | 4 | 7.33 | Normal | Normal | CD9 | 12p13 | CD9 antigen (p24) | CD9 antigen (p24) |
| CASM | AF000177_at | AF000177 | fail | 4 | | PASS | 9 | 4 | 7.11 | Normal | Normal | CaSm | | CaSm | Sm-like protein; encodes |
| | | | | | | | | | | | | | | | Sm motifs; overexpressed |
| | | | | | | | | | | | | | | | in pancreatic cancer |
| K138_THH | D50928_at | D50928 | fail | 4 | | PASS | 9 | 4 | 7.00 | Normal | Normal | KIAA0138 | | | KIAA0138 gene product |
| PHBLN1 | U03891_at | U03891 | fail | 4 | | PASS | 9 | 4 | 5.89 | Normal | Normal | DJ742C19.2 | 22q12.3-q13.1 | phorbolin (similar to | phorbolin (similar to |
| | | | | | | | | | | | | | | apolipoprotein B mRNA | apolipoprotein B mRNA |
| | | | | | | | | | | | | | | editing protein) | editing protein) |
| HLA-DPB1 | M83664_at | M83664 | fail | 4 | | PASS | 9 | 4 | 5.67 | Normal | Normal | HLA-DPB1 | | HLA-DPB1 | |
| K251_COSC | D87438_at | D87438 | fail | 4 | | PASS | 9 | 4 | 5.44 | Normal | Normal | KIAA0251 | | | Similar to a C. elegans |
| | | | | | | | | | | | | | | protein in cosmid C14H10 | |
| SORD | L29008_at | L29008 | fail | 4 | | PASS | 9 | 4 | 5.33 | Normal | Normal | SORD | 15q15-q21.1 | sorbitol dehydrogenase | sorbitol dehydrogenase |
| K92_MYH6 | D42054_at | D42054 | fail | 4 | | PASS | 9 | 4 | 5.22 | Normal | Normal | KIAA0092 | | | KIAA0092 gene product |
| RECA | L07493_at | L07493 | fail | 4 | | PASS | 9 | 4 | 4.89 | Normal | Normal | RPA3 | 7 | replication protein A3 | replication protein A3 |
| | | | | | | | | | | | | | | (14 kD) | (14 kD) |
| SCP2 | U11313_at | U11313 | fail | 4 | | PASS | 9 | 4 | 4.89 | Normal | Normal | SCP2 | 1pter-p21 | sterol carrier protein 2 | sterol carrier protein 2 |
| YAF2 | U72209_at | U72209 | fail | 4 | | PASS | 9 | 4 | 4.78 | Normal | Normal | YAF2 | | YY1-associated factor 2 | zinc finger protein |
| PPP2R1B | M65254_at | M65254 | fail | 4 | | PASS | 9 | 4 | 4.56 | Normal | Normal | PPP2R1B | 11q23 | protein phosphatase-2A | protein phosphatase 2 |
| | | | | | | | | | | | | | | regulatory subunit-beta | (formerly 2A), regulatory |
| | | | | | | | | | | | | | | | subunit A (PR 65), beta |
| | | | | | | | | | | | | | | | isoform |
| CD94 | U30610_at | U30610 | fail | 3 | | PASS | 9 | 3 | 8.44 | Normal | Normal | KLRD1 | 12p13 | killer cell lectin-like | killer cell lectin-like |
| | | | | | | | | | | | | | | receptor subfamily D, | receptor subfamily D, |
| | | | | | | | | | | | | | | member 1 | member 1 |
| SNRPB2 | M15841_at | M15841 | fail | 3 | | PASS | 9 | 3 | 744 | Normal | Normal | SNRPB2 | | small nuclear ribonucleo- | small nuclear ribonucleo- |
| | | | | | | | | | | | | | | protein polypeptide B″ | protein polypeptide B″ |
| CLC | L01664_at | L01664 | fail | 3 | | PASS | 9 | 3 | 7.22 | Normal | Normal | CLC | 19q13.1 | Charot-Leyden crystal | Charot-Leyden crystal |
| | | | | | | | | | | | | | | protein | protein |
| AFFX-HUM | AFFX-HUM | APFX-HUM | fail | 3 | | PASS | 9 | 3 | 7.11 | Normal | Normal | | | | |
| SNX1 | U53225_at | U53225 | fail | 3 | | PASS | 9 | 3 | 6.67 | Normal | Normal | SNX1 | | sorting nexin 1 | sorting nexin 1 |
| FKBP5 | U42031_at | U42031 | fail | 3 | | PASS | 9 | 3 | 6.56 | Normal | Normal | FKB5 | | FK506-binding protein 5 | FK506-binding protein 5 |
| S72024_s_at | S72024_s_at | S72024 | fail | 3 | | PASS | 9 | 3 | 6.44 | Normal | Normal | eif-5A | | | eukaryotic initiation factor |
| | | | | | | | | | | | | | | | 5A |
| SMARCC1 | U66615_at | U66615 | fail | 3 | | PASS | 9 | 3 | 5.67 | Normal | Normal | SMARCC1 | 3p23-p21 | SWI/SNF related, matrix | SWI/SNF related, matrix |
| | | | | | | | | | | | | | | associated, actin dependent | associated, actin dependent |
| | | | | | | | | | | | | | | reulator of chromatin, | regulator of chromatin, |
| | | | | | | | | | | | | | | subfamily c, member 1 | subfamily c, member 1 |
| L05624_s_at | L05624_s_at | L05624 | fail | 3 | | PASS | 9 | 3 | 5.56 | Normal | Normal | | | MAP kinase kinase | |
| TPP2 | M73047_at | M73047 | fail | 3 | | PASS | 9 | 3 | 5.56 | Normal | Normal | TPP2 | 13q32-q33 | tripeptidyl peptidase II | tripeptidyl peptidase II |
| PLK | U01038_at | U01038 | fail | 3 | | PASS | 9 | 3 | 5.56 | Normal | Normal | | | pLK | |
| STAT6 | U16031_at | U16031 | fail | 3 | | PASS | 9 | 3 | 5.56 | Normal | Normal | STAT6 | 12q13 | signal transducer and | signal transducer and |
| | | | | | | | | | | | | | | activator of transcription | activator of transcription |
| | | | | | | | | | | | | | | 6, interleukin-4 induced | 6, interleukin-4 induced |
| NUP88 | Y08612_at | Y08612 | fail | 3 | | PASS | 9 | 3 | 5.44 | Normal | Normal | NUP88 | 17p13 | nucleoporin 88 kD | nucleoporin 88 kD |
| S79219_s_at | S79219_s_at | S79219 | fail | 3 | | PASS | 9 | 3 | 5.22 | Normal | Normal | PCCA | 13q32 | propionyl Coenzyme A | propionyl Coenzyme A |
| | | | | | | | | | | | | | | carboxylase, alpha | carboxylase, alpha |
| | | | | | | | | | | | | | | polypeptide | polypeptide |
| C2F | U72514_at | U72514 | fail | 3 | | PASS | 9 | 3 | 5.00 | Normal | Normal | C2f | | C2f | similar to EST with |
| | | | | | | | | | | | | | | | GenBank Accession |
| | | | | | | | | | | | | | | | Number R64505; similar to |
| | | | | | | | | | | | | | | | S. cerevisiae hypothetical |
| | | | | | | | | | | | | | | | protein L9470.5 encoded by |
| | | | | | | | | | | | | | | | GenBank Accession |
| | | | | | | | | | | | | | | | Number S51431, and to S. |
| | | | | | | | | | | | | | | | pombe hypothetical |
| | | | | | | | | | | | | | | | 34.9KD protein encoded by |
| | | | | | | | | | | | | | | | GenBank Accession |
| | | | | | | | | | | | | | | | Number Z68198; see |
| | | | | | | | | | | | | | | | corresponding genomic |
| | | | | | | | | | | | | | | | sequence in GenBank |
| | | | | | | | | | | | | | | | Accession Number U72506 |
| E_23733 | U79274_at | U79274 | fail | 3 | | PASS | 9 | 3 | 5.00 | Normal | Normal | | | | |
| DCK | M60527_at | M60527 | fail | 3 | | PASS | 9 | 3 | 4.67 | Normal | Normal | DCK | 4q13.3-q21.l | deoxycytidine kinase | deoxycytidine kinase |
| AFFX-BioB-1 | XPFX-BioB-1 | AFFX-BioB | fail | 0 | | PASS | 9 | 0 | 17.44 | Normal | Normal | | | | |
| RE5424A | AB000464_at | AB000464 | fail | 4 | | PASS | 8 | 4 | 8.50 | Normal | Normal | | | | |
| SSRP1 | M86737_at | M86737 | fail | 4 | | PASS | 8 | 4 | 7.75 | Normal | Normal | SSRP1 | 11q12 | structure specific | structure specific |
| | | | | | | | | | | | | | | recognition protein 1 | recognition protein 1 |
| BAT3 | M33521_at | M33521 | fail | 4 | | PASS | 8 | 4 | 7.50 | Normal | Normal | D6SS52E | 6p21.3 | HLA-B associated | HLA-B associated |
| | | | | | | | | | | | | | | transcript-3 | transcript-3 |
| PNOC | U48263_at | U48263 | fail | 4 | | PASS | 8 | 4 | 7.00 | Normal | Normal | OFQ | | pre-pro-orphanin FA | |
| ERCC1 | M13194_at | M13194 | fail | 4 | | PASS | 8 | 4 | 6.63 | Normal | Normal | ERCC1 | 19q13.2-q13.3 | excision repair cross- | excision repair cross- |
| | | | | | | | | | | | | | | complementing rodent | complementing rodent |
| | | | | | | | | | | | | | | repair deficiency, | repair deficiency, |
| | | | | | | | | | | | | | | complementation group 1 | complementation group 1 |
| | | | | | | | | | | | | | | (includes overlapping | (includes overlapping |
| | | | | | | | | | | | | | | antisense sequence) | antisense sequence) |
| TRNAGUTR | U30888_at | U30888 | fail | 4 | | PASS | 8 | 4 | 6.38 | Normal | Normal | USP14 | | tRNA-Guanine | ubiquitin specific protease |
| | | | | | | | | | | | | | | Transglycosylase | 14 (tRNA-guanine trans- |
| | | | | | | | | | | | | | | | glycosylase) |
| L24774_s_at | L24774_s _at | L24774 | fail | 4 | | PASS | 8 | 4 | 5.88 | Normal | Normal | DCI | 16p13.3 | dodecenoyl-Coenzyme A | dodecenoyl-Coenzyme A |
| | | | | | | | | | | | | | | delta isomerase (3,2 | delta isomerase (3,2 |
| | | | | | | | | | | | | | | trans-enoyl-Coenzyme | trans-enoyl-Coenzyme |
| | | | | | | | | | | | | | | A isomerase) | A isomerase) |
| HG33l9-HT3 | Ht33319-HT3 | HG3319-HT | fail | 4 | | PASS | 8 | 4 | 5.63 | Normal | Normal | | | | |
| G9 | X78687_at | X78687 | fail | 4 | | PASS | 8 | 4 | 5.38 | Normal | Normal | NEu | 6 or 10pter- | neuraminidase | neuraminidase |
| | | | | | | | | | | | | | q23 | | |
| ZNF139 | U09848_at | U09848 | fail | 4 | | PASS | 8 | 4 | 5.00 | Normal | Normal | ZNF139 | | zinc finger protein | zinc finger protein |
| | | | | | | | | | | | | | | | 139 (clone pHZ-37) |
| ZFX | X59739_at | X59739 | fail | 4 | | PASS | 8 | 4 | 5.00 | Normal | Normal | ZFX | Xp22.1-q21.3 | zinc finger protein, | zinc finger protein, |
| | | | | | | | | | | | | | | X-linked | X-linked |
| S80267_s_at | S80267_s_at | S80267 | fail | 4 | | PASS | 8 | 4 | 4.88 | Normal | Normal | p72syk | | p72syk | This sequence comes from |
| | | | | | | | | | | | | | | | FIG. 3. |
| DARS | J05032_at | J05032 | fail | 4 | | PASS | 8 | 4 | 4.63 | Normal | Normal | DARS | | aspartyl-tRNA synthetase | aspartyl-tRNA synthetase |
| U18271_cds | U18271_cds3 | U18271 | fail | 4 | | PASS | 8 | 4 | 4.63 | Normal | Normal | TMPO | | thymopoitin beta | |
| K128_HCDC | D50918_at | D50918 | fail | 3 | | PASS | 8 | 3 | 7.13 | Normal | Normal | KIAA0128 | | | The KIAA0128 gene is |
| | | | | | | | | | | | | | | | related to cdc10. |
| TDRAA | HG3578-HT3 | HG3578-HT | fail | 3 | | PASS | 8 | 3 | 6.25 | Normal | Normal | | | | |
| LAMB2 | M94362_at | M94362 | fail | 3 | | PASS | 8 | 3 | 6.00 | Normal | Normal | LAMB2 | | lamin B2 | |
| SH3BP2 | U32519_at | U32519 | fail | 3 | | PASS | 8 | 3 | 5.50 | Normal | Normal | | | GAP SH3 binding protein | |
| MURR1 | D85433_at | D85433 | fail | 3 | | PASS | 8 | 3 | 5.38 | Normal | Normal | | | | |
| ITGAE | L25851_at | L25851 | fail | 3 | | PASS | 8 | 3 | 5.25 | Normal | Normal | ITGAE | | integrin, alpha E (antigen | integrin, alpha E (antigen |
| | | | | | | | | | | | | | | CD103, human mucosal | CD103, human mucosal |
| | | | | | | | | | | | | | | lymphocyte antigen 1; | lymphocyte antigen 1; |
| | | | | | | | | | | | | | | alpha polypeptide) | alpha polypeptide) |
| SPLREGSUP | U08377_at | U08377 | fail | 3 | | PASS | 8 | 3 | 5.25 | Normal | Normal | SFRS8 | | splicing factor, arginine/ | splicing factor, arginine/ |
| | | | | | | | | | | | | | | serine-rich 8 (suppressor- | serine-rich 8 (suppressor- |
| | | | | | | | | | | | | | | of-white-apricot, | of-white-apricot, |
| | | | | | | | | | | | | | | Drosophila homolog) | Drosophila homolog) |
| STX16C | AF008937_at | AF008937 | fail | 3 | | PASS | 8 | 3 | 5.00 | Normal | Normal | STX16 | | syntaxin 16 | syntaxin 16 |
| ANX3B | U37139_at | U37139 | fail | 3 | | PASS | 8 | 3 | 4.88 | Normal | Normal | | | beta 3-endonexin long form | The long form does not |
| | | | | | | | | | | | | | | | bind to the integrin |
| | | | | | | | | | | | | | | | beta 3 subunit |
| | | | | | | | | | | | | | | | sytoplasmic domain |
| K33 | D26067_at | D26067 | fail | 3 | | PASS | 8 | 3 | 4.75 | Normal | Normal | KIAA0033 | | | |
| AHR | L19872_at | L19872 | fail | 3 | | PASS | 8 | 3 | 4.75 | Normal | Normal | AHR | 7p15 | aryl hydrocarbon receptor | aryl hydrocarbon receptor |
| MEST | D78611_at | D78611 | fail | 3 | | PASS | 8 | 3 | 4.38 | Normal | Normal | MEST | 7q32 | mesoderm specific | mesoderm specific |
| | | | | | | | | | | | | | | transcript (mouse) homolog | transcript (mouse) homolog |
| PIGH | L19783_at | L19783 | fail | 3 | | PASS | 8 | 3 | 4.38 | Normal | Normal | PIGH | 14q11-q24 | phosphatidylinositol glycan, | phosphatidylinositol glycan |
| | | | | | | | | | | | | | | class H | class H |
| M34338_s_at | M34338_s_at | M34338 | fail | 2 | | PASS | 8 | 2 | 7.50 | Normal | Normal | SRM | 1p36-p22 | spermidine sythase | spermidine synthase |
| WWP1 | U96113_at | U96113 | fail | 2 | | PASS | 8 | 2 | 5.88 | Normal | Normal | | | WWP1 | Nedd-4-like ubiquitin- |
| | | | | | | | | | | | | | | | protein ligase; WW |
| | | | | | | | | | | | | | | | domain-containing protein |
| RH17599 | L06175_at | L06175 | fail | 2 | | PASS | 8 | 2 | 5.75 | Normal | Normal | P5-1 | | | occurs in MHC class 1 |
| | | | | | | | | | | | | | | | region; ORF |
| ITBA1 | X92475_at | X92475 | fail | 2 | | PASS | 8 | 2 | 5.75 | Normal | Normal | ITBA1 | Xg28 | ITBA1 protein | ITBA1 gene |
| CL1042 | X70649_at | X70649 | fail | 2 | | PASS | 8 | 2 | 5.38 | Normal | Normal | DDX1 | 2p24 | member of DEAD box | DEAD/H (Asp-Gln- |
| | | | | | | | | | | | | | | protein family | Ala-Asp/His) box |
| | | | | | | | | | | | | | | | polypeptide 1 |
| HG3417-HT3 | HG3417-HT3 | HG3417-HT | fail | 2 | | PASS | 8 | 2 | 5.25 | Normal | Normal | | | | |
| TACT | M88282_at | M88282 | fail | 2 | | PASS | 8 | 2 | 5.25 | Normal | Normal | | | tactile protein | T cell surface antigen, |
| | | | | | | | | | | | | | | | increased during activation′ |
| ZFP2 | U71598_at | U71598 | fail | 2 | | PASS | 8 | 2 | 5.25 | Normal | Normal | zf2 | | zinc finger protein zfp2 | |
| K116_75KD | D29958_at | D29958 | fail | 2 | | PASS | 8 | 2 | 5.13 | Nonoal | Normal | KIAA0116 | | | |
| IMOG38 | Z68747_at | Z68747 | fail | 2 | | PASS | 8 | 2 | 5.13 | Normal | Normal | | | imogen 38 | proprotein |
| BTG3 | D64110_at | D64110 | fail | 2 | | PASS | 8 | 2 | 5.00 | Normal | Normal | | | ANA | |
| RATC10 | HG1879-HT1 | HG1879-HT | fail | 2 | | PASS | 8 | 2 | 5.00 | Normal | Normal | | | | |
| GCF | M29204_at | M29204 | fail | 2 | | PASS | 8 | 2 | 5.00 | Normal | Normal | TCF9 | 2p11.2-p11.1 | transcription factor 9 | transcription factor 9 |
| | | | | | | | | | | | | | | (binds GC-rich sequences) | (binds GC-rich sequences) |
| HG3546-HT3 | HG3546-HT3 | HG3546-HT | fail | 2 | | PASS | 8 | 2 | 4.88 | Normal | Normal | | | | |
| ATF1 | X55544_at | X55544 | fail | 2 | | PASS | 8 | 2 | 4.75 | Normal | Normal | ATF1 | 12q13 | TREB protein | activating transcription |
| | | | | | | | | | | | | | | | factor 1 |
| UNKP | U50950_at | U50950 | fail | 2 | | PASS | 8 | 2 | 4.13 | Normal | Normal | | | | |
| ALDOC | X05196_at | X05196 | fail | 1 | | PASS | 8 | 1 | 7.00 | Normal | Normal | ALDOC | 17cen-q12 | aldolase C | aldolase C, fructose- |
| | | | | | | | | | | | | | | | bisphosphate |
| SEC23B | X97065_at | X97065 | fail | 1 | | PASS | 8 | 1 | 6.13 | Normal | Normal | sec23 | | Sec23 protein | COPII component; |
| | | | | | | | | | | | | | | | isoform B |
| GTF2B | M76766_at | M76766 | fail | 1 | | PASS | 8 | 1 | 5.88 | Normal | Normal | GTF2B | 1p22-p21 | general transcription | general transcription |
| | | | | | | | | | | | | | | factor IIB | factor IIB |
| RPP30 | U77665_at | U77665 | fail | 1 | | PASS | 8 | 1 | 5.63 | Normal | Normal | RPP30 | | RNaseP protein P30 | |
| COASYNT | Z68204_at | Z68204 | fail | 1 | | PASS | 8 | 1 | 5.50 | Normal | Normal | SUCLG1 | | succinyl coenzyme A | succinate-CoA ligase, |
| | | | | | | | | | | | | | | synthetase | GDP-forming, alpha |
| | | | | | | | | | | | | | | | subunit |
| DDX3 | U50553_at | U50553 | fail | 1 | | PASS | 8 | 1 | 5.13 | Normal | Normal | DDX3 | Xp11.3-p11.23 | DEAD/H (Asp-Glu-Ala- | DEAD/H (Asp-Glu-Ala- |
| | | | | | | | | | | | | | | Asp/His) box polypeptide 3 | Asp/His) box polypeptide 3 |
| SFRS2 | HG3088-HT3 | HG3088-HT | fail | 1 | | PASS | 8 | 1 | 4.75 | Normal | Normal | | | | |
| CD1C | M28827_at | M28827 | fail | 1 | | PASS | 8 | 1 | 4.75 | Normal | Normal | CD1C | 1q22-q23 | CD1C antigen, c poly- | CD1C antigen, c poly- |
| | | | | | | | | | | | | | | peptide | peptide |
| BTF2 | M95809_at | M95809 | fail | 1 | | PASS | 8 | 1 | 4.75 | Normal | Normal | BTF2 | | basic transcription | |
| | | | | | | | | | | | | | | factor 62 kD subunit | |
| TSNAX | X95073_at | X95073 | fail | 1 | | PASS | 8 | 1 | 4.75 | Normal | Normal | TRAX | | Translin associated protein | |
| | | | | | | | | | | | | | | X | |
| CSNK1A1 | L37042_at | L37042 | fail | 4 | | PASS | 7 | 4 | 8.00 | Normal | Normal | CSNK1A1 | 13q13 | casein kinase 1, alpha 1 | casein kinase 1, alpha 1 |
| PLCD1 | U09117_at | U09117 | fail | 4 | | PASS | 7 | 4 | 7.57 | Normal | Normal | | | phospholipase c delta 1 | |
| S10 | D14889_at | D14889 | fail | 4 | | PASS | 7 | 4 | 5.71 | Normal | Normal | RAB33A | X | RAB33A, member RAS | RAB33A, member RAS |
| | | | | | | | | | | | | | | oncogene family | oncogene family |
| AEBP1 | D86479_at | D86479 | fail | 4 | | PASS | 7 | 4 | 5.71 | Normal | Normal | AEBPI | 7 | AE-binding protein 1 | AE-binding protein 1 |
| SKB1 | AF015913_at | AF015913 | fail | 4 | | PASS | 7 | 4 | 5.57 | Normal | Normal | SKB1Hs | | Skb1Hs | homolog of fission yeast |
| | | | | | | | | | | | | | | | Skb1 |
| K03498_xpt1 | K03498_xpt1 | K03498 | fail | 4 | | PASS | 7 | 4 | 5.57 | Normal | Normal | | | pol protein | env ORF (bases 110-& gt; |
| | | | | | | | | | | | | | | | 290) first start codon at |
| | | | | | | | | | | | | | | | 191; putative |
| SL1 | L39059_at | L39059 | fail | 4 | | PASS | 7 | 4 | 5.43 | Normal | Normal | | | transcription factor SL1 | |
| PIGF | D13435_at | D13435 | fail | 4 | | PASS | 7 | 4 | 5.29 | Normal | Normal | PIGF | 2p21-p16 | phosphatidyinositol glycan, | phosphatidyinositol glycan, |
| | | | | | | | | | | | | | | class F | class F |
| TCF4 | M74719_at | M747l9 | fail | 4 | | PASS | 7 | 4 | 5.14 | Normal | Normal | TCF4 | 18q21.1 | transcription factor 4 | transcription factor 4 |
| GOLGA1 | U51S87_at | U51587 | fail | 4 | | PASS | 7 | 4 | 5.14 | Normal | Normal | GOLGA1 | golgin 97 | golgi autoantigen, golgin | |
| | | | | | | | | | | | | | | | subfamily a, 1 |
| X75091_s_at | X75091_s_at | X75091 | fail | 4 | | PASS | 7 | 4 | 5.00 | Normal | Normal | | | PHAPII (Putative HLA DR | |
| | | | | | | | | | | | | | | Associated Protein II) | |
| POLR3 | U93867_at | U93867 | fail | 4 | | PASS | 7 | 4 | 4.71 | Normal | Normal | RPC62 | | RNA polymerase III | |
| | | | | | | | | | | | | | | subunit | |
| GLCLC | M90656_at | M90656 | fail | 4 | | PASS | 7 | 4 | 4.43 | Normal | Normal | GLCLC | 6p12 | glutamate-cysteine ligase | glutamate-cystein ligase |
| | | | | | | | | | | | | | | | (gamma-glutamylcysteine |
| | | | | | | | | | | | | | | | synthetase), catalytic |
| | | | | | | | | | | | | | | | (72.8 kD) |
| PTPRE | HG620-HT62 | HG620-HT | fail | 3 | | PASS | 7 | 3 | 8.00 | Normal | Normal | | | | |
| OBRGRP | Y12670_at | Y12670 | fail | 3 | | PASS | 7 | 3 | 8.00 | Normal | Normal | OB-RGRP | | leptin receptor gene- | |
| | | | | | | | | | | | | | | related protein | |
| DRNM23 | U29656_at | U29656 | fail | 3 | | PASS | 7 | 3 | 7.29 | Normal | Normal | NME3 | 16q13 | non-metastatic cells 3, | non-metastatic cells 3, |
| | | | | | | | | | | | | | | protein expressed in | protein expressed in |
| OMD | AB000114_at | AB000114 | fail | 3 | | PASS | 7 | 3 | 6.86 | Normal | Normal | OMB | | osteomodulin | osteomodulin |
| RY1 | X76302_at | X76302 | fail | 3 | | PASS | 7 | 3 | 6.86 | Normal | Normal | RY-1 | | nucleic acid binding protein | |
| K12_TOLL | D13637_at | D13637 | fail | 3 | | PASS | 7 | 3 | 6.57 | Normal | Normal | TLR1 | 4p14 | toll-like receptor 1 | toll-like receptor 1 |
| CSNK2A1 | M55265_at | M55265 | fail | 3 | | PASS | 7 | 3 | 5.86 | Normal | Normal | CSNK2A1 | 20p13 | casein kinase 2, alpha 1 | casein kinase 2, alpha 1 |
| | | | | | | | | | | | | | | polypeptide | polypeptide |
| DOC2 | U53446_at | U53446 | fail | 3 | | PASS | 7 | 3 | 5.86 | Normal | Normal | DAB2 | 6p23 | disabled (Drosophila) | disabled (Drosphila) |
| | | | | | | | | | | | | | | homolog 2 (mitogen- | homolog 2 (mitogen- |
| | | | | | | | | | | | | | | responsive phosphoprotein) | responsive phosphoprotein) |
| SMARCD1 | U66617_at | U66617 | fail | 3 | | PASS | 7 | 3 | 5.71 | Normal | Normal | SMARCD1 | 12q13-q14 | SWI/SNF related, matrix | SWI/SNF related, matrix |
| | | | | | | | | | | | | | | associated, actin dependent | associated, actin dependent |
| | | | | | | | | | | | | | | regulator of chromatin, | regulator of chromatin, |
| | | | | | | | | | | | | | | subfamily d, member 1 | subfamily d, member 1 |
| ZNF131 | U09410_at | U09410 | fail | 3 | | PASS | 7 | 3 | 5.43 | Normal | Normal | ZNF232 | | zinc finger protein ZNF131 | zinc finger protein 131 |
| | | | | | | | | | | | | | | | (clone pIIZ-10) |
| GLIPR | U16307_at | U16307 | fail | 3 | | PASS | 7 | 3 | 5.43 | Normal | Normal | GIiPR | | glioma pathogenesis- | |
| | | | | | | | | | | | | | | related protein | |
| E2F5 | U31556_at | U31556 | fail | 3 | | PASS | 7 | 3 | 5.43 | Normal | Normal | | | E2F-5 | transcription factor |
| BARDL1 | U76638_at | U76638 | fail | 3 | | PASS | 7 | 3 | 4.29 | Normal | Normal | BARDI | 2q34-q35 | BRCA1 associated RING | BRCA1 associated RING |
| | | | | | | | | | | | | | | domain 1 | domain 1 |
| U40763_s_at | U40763_s_at | U40763 | fail | 3 | | PASS | 7 | 3 | 4.14 | Normal | Normal | CYP | | Clk-associating RS- | Clk-associating RS- |
| | | | | | | | | | | | | | | cyclophilin | cyclophilin |
| ARH9 | L25081_at | L25081 | fail | 2 | | PASS | 7 | 2 | 8.86 | Normal | Normal | ARHC | 1p21-p13 | GTPase | ras homolog gene family, |
| | | | | | | | | | | | | | | | member C |
| HSN | U03057_at | U03057 | fail | 2 | | PASS | 7 | 2 | 8.86 | Normal | Normal | SNL | 7p22 | singed (Drosophila)-like | singed (Drosophila)-like |
| | | | | | | | | | | | | | | (sea urchin fascin homolog | (sea urchin fascin homolog |
| | | | | | | | | | | | | | | like) | like) |
| TARS | M63180_at | M63180 | fail | 2 | | PASS | 7 | 2 | 5.57 | Normal | Normal | TARS | 5p13-cen | threonyl-tRNA synthetase | threonl-tRNA synthetase |
| K182 | U80004_at | U80004 | fail | 2 | | PASS | 7 | 2 | 5.43 | Normal | Normal | KIAA0182 | | | |
| TFAP3B | U91931_at | U91931 | fail | 2 | | PASS | 7 | 2 | 5.43 | Normal | Normal | ADTB3 | | Beta-3A-adaptin | Beta-3A-adaptin |
| CTIP | U72066_at | U72066 | fail | 2 | | PASS | 7 | 2 | 5.14 | Normal | Normal | RBBP8 | 18q11.2 | retinoblastoma-binding | retinoblastoma-binding |
| | | | | | | | | | | | | | | protein 8 | protein 8 |
| CD72 | M54992_at | M54992 | fail | 2 | | PASS | 7 | 2 | 5.00 | Normal | Normal | CD72 | 9p | CD72 antigen | CD72 antigen |
| PDE6B | S41458_at | S41458 | fail | 2 | | PASS | 7 | 2 | 4.86 | Normal | Normal | PDE6B | 4p16.3 | phosphodiesterase 6B, | phosphodiesterase 6B, |
| | | | | | | | | | | | | | | cGMP-specific, rod, beta | cGMP-specific, rod, beta |
| | | | | | | | | | | | | | | (congential stationary | (congential stationary |
| | | | | | | | | | | | | | | night blindness 3, | night blindness 3, |
| | | | | | | | | | | | | | | autosomal dominant) | autosomal dominant) |
| GTBP | U73737_at | U73737 | fail | 2 | | PASS | 7 | 2 | 4.86 | Normal | Normal | GTBP | 2p26 | G/T mismatch-binding | G/T mismatch binding |
| | | | | | | | | | | | | | | protein | protein |
| U67932 a_at | U67932 a_at | U67932 | fail | 2 | | PASS | 7 | 2 | 4.57 | Normal | Normal | Pde7A2 | | cAMP phosphodiesterase | |
| GBE1 | L07956_at | L07956 | fail | 2 | | PASS | 7 | 2 | 4.43 | Normal | Normal | GBE1 | 3p21 | glucan (1,4-alpha-), | glucan (1,4-alpha-), |
| | | | | | | | | | | | | | | enzyme 1 (glycogen | enzyme 1 (glycogen |
| | | | | | | | | | | | | | | branching enzyme, | branching enzyme, |
| | | | | | | | | | | | | | | Andersen disease, glycogen | Andersen disease, glycogen |
| | | | | | | | | | | | | | | glycogen storage disease | glycogen storage disease |
| | | | | | | | | | | | | | | type IV) | type IV) |
| CDR2 | M63256_at | M63256 | fail | 2 | | PASS | 7 | 2 | 4.43 | Normal | Normal | CDR2 | 16p13.1-p12 | major Yo paraneoplastic | cerebellar degeneration- |
| | | | | | | | | | | | | | | antigen | related protein (62 kD) |
| RP1H | U90437_at | U90437 | fail | 2 | | PASS | 7 | 2 | 4.29 | Normal | Normal | | | | |
| HNRPA0 | U23803_at | U23803 | fail | 1 | | PASS | 7 | 1 | 8.71 | Normal | Normal | | | heterogeneous | hnRNP protein; hnRNA |
| | | | | | | | | | | | | | | ribonucleoprotein A0 | binding protein |
| ICRF | Z69915_at | Z69915 | fail | 1 | | PASS | 7 | 1 | 6.00 | Normal | Normal | | | | |
| SSR1 | Z12830_at | Z12830 | fail | 1 | | PASS | 7 | 1 | 5.86 | Normal | Normal | SSR1 | | signal sequence receptor, | signal sequence receptor, |
| | | | | | | | | | | | | | | alpha (translocon- | alpha (translocon- |
| | | | | | | | | | | | | | | associated protein alpha) | associated protein alpha) |
| RALGDS | U14417_at | U14417 | fail | 1 | | PASS | 7 | 1 | 5.71 | Normal | Normal | | | Ral guanine nucleotide | the C-terminal non-catalytic |
| | | | | | | | | | | | | | | dissociation stimulator | domain of the Ral GDS |
| | | | | | | | | | | | | | | | interacts with Ras |
| SMARCC2 | U66616_at | U66616 | fail | 1 | | PASS | 7 | 1 | 5.71 | Normal | Normal | SMARCC2 | 12q13-q14 | SWI/SNF related, matrix | SWI/SNF related, matrix |
| | | | | | | | | | | | | | | associated, actin dependent | associated, actin dependent |
| | | | | | | | | | | | | | | regulator of chromatin, | regulator of chromatin, |
| | | | | | | | | | | | | | | subfamily c, member 2 | subfamily c, member 2 |
| PCCB | S67325_at | S67325 | fail | 1 | | PASS | 7 | 1 | 5.29 | Normal | Normal | PCCB | 3q21-q22 | propionyl Coenzyme A | propionyl Coenzyme A |
| | | | | | | | | | | | | | | carboxylase, beta | carboxylase, beta |
| | | | | | | | | | | | | | | polypeptide | polypeptide |
| G16P | U50839_at | U50839 | fail | 1 | | PASS | 7 | 1 | 5.00 | Normal | Normal | g16 | | g16 protein | |
| CUL2 | U83410_at | U83410 | fail | 1 | | PASS | 7 | 1 | 5.00 | Normal | Normal | CUL2 | | cullin 2 | cullin 2 |
| CEBPG | U20240_at | U20240 | fail | 1 | | PASS | 7 | 1 | 4.86 | Normal | Normal | CEBPG | | CCAAT/enhancer binding | CCAAT/enhancer binding |
| | | | | | | | | | | | | | | protein (C/EBP), gamma | protein (C/EBP), gamma |
| RAB9 | U44103_at | U44103 | fail | 1 | | PASS | 7 | 1 | 4.86 | Normal | Normal | RAB9 | | RAB9, member RAS | RAB9, member RAS |
| | | | | | | | | | | | | | | oncogene family | oncogene family |
| KSS TRE | D29956_at | D29956 | fail | 1 | | PASS | 7 | 1 | 4.43 | Normal | Normal | USP8 | | | |
| CRIP2 | D42123_at | D42123 | PASS | 9 | 25.56 | fail | 5 | 9 | | Disease | Disease | CRIP2 | 14g32.3 | cysteine-rich protein 2 | cysteine-rich protein 2 |
| K14 | D25216_at | D25216 | PASS | 9 | 31.67 | fail | 5 | 9 | | Disease | Disease | KIAA0014 | | | KIAA0014 gene product |
| LTK | D16105_at | D16105 | PASS | 9 | 23.78 | fail | 3 | 9 | | Disease | Disease | LTK | 15 | leukocyte tyrosine kinase | leukocyte tyrosine kinase |
| K60_GNPTA | D31766_at | D31766 | PASS | 8 | 14.50 | fail | 6 | 8 | | Disease | Disease | KIAA0060 | | | KIAA0060 gene product |
| CDK2 | M68520_at | M68520 | PASS | 8 | 7.63 | fail | 6 | 8 | | Disease | Disease | CDK2 | 12q13 | cyclin-dependent kinase 2 | cyclin-dependent kinase 2 |
| IL11RA | U32324_at | U32324 | PASS | 8 | 9.75 | fail | 6 | 8 | | Disease | Disease | IL22RA | 9p13 | interleukin 11 receptor, | interleukin 11 receptor, |
| | | | | | | | | | | | | | | alpha | alpha |
| ACP2_rna1 | X15525_rna1 | X15525 | PASS | 8 | 8.75 | fail | 6 | 8 | | Disease | Disease | ACP2 | | acid phosphatase | |
| H326 | U06631_at | U06631 | PASS | 8 | 23.13 | fail | 5 | 8 | | Disease | Disease | H326 | | | homologous to mouse gene |
| | | | | | | | | | | | | | | | PC326:GenBank Accession |
| | | | | | | | | | | | | | | | Number M95564 |
| TEB4 | AF009301_at | AF009301 | PASS | 8 | 9.88 | fail | 5 | 8 | | Disease | Disease | | TEB4 protein | | |
| K170_CALT | D79992_at | D79992 | PASS | 8 | 6.88 | fail | 5 | 8 | | Disease | Disease | KIAA0170 | | | KIAA0170 gene product |
| PRKACG | M34182_at | M34182 | PASS | 8 | 41.00 | fail | 5 | 8 | | Disease | Disease | PRKACG | 9q13 | protein kinase, cAMP- | protein kinase, cAMP- |
| | | | | | | | | | | | | | | dependent, catalytic, | dependent, catalytic, |
| | | | | | | | | | | | | | | gamma | gamma |
| PLEC1 | U3204_at | U53204 | PASS | 8 | 40.38 | fail | 4 | 8 | | Disease | Disease | PLEC1 | 8q24 | protein 1, intermediate | protein 1, imtermediate |
| | | | | | | | | | | | | | | filament binding protein, | filament binding protein, |
| | | | | | | | | | | | | | | 500 kD | 500 kD |
| E_LF113 | U18009_at | U18009 | PASS | 8 | 13.00 | fail | 4 | 8 | | Disease | Disease | | | | similar to Pacific ray |
| | | | | | | | | | | | | | | | VAT1 protein, Swiss-Prot |
| | | | | | | | | | | | | | | | Accession Number P19333 |
| 18SRNA5 | AFFX-HUM | AFFX-HUM | PASS | 8 | 12.00 | fail | 3 | 8 | | Disease | Disease | | | | |
| MUC3 | HG2147-HT2 | H02147-HT | PASS | 8 | 60.63 | fail | 3 | 8 | | Disease | Disease | | | | |
| MYBPC3 | X73113_at | X73113 | PASS | 8 | 11.25 | fail | 3 | 8 | | Disease | Disease | MYBPC2 | 19 | myosin-binding protein | myosin-binding protein |
| | | | | | | | | | | | | | | C, fast-type | C, fast-type |
| NB | D89016_at | D89016 | PASS | 8 | 20.63 | fail | 2 | 8 | | Disease | Disease | nbr | | Neuroblastoma | |
| U47025 a_at | U47025_s_at | U47025 | PASS | 7 | 27.43 | fail | 6 | 7 | | Disease | Disease | PYGB | 20 | phosphorylase, glycogen; | phosphorylase, glycogen; |
| | | | | | | | | | | | | | | brain | brain |
| KIDDNABP | D45132_at | D45132 | PASS | 7 | 5.86 | fail | 6 | 7 | | Disease | Disease | | | zinc-finger DNA-binding | |
| | | | | | | | | | | | | | | protein | |
| K179_HYPD | D80001_at | D80001 | PASS | 7 | 8.00 | fail | 6 | 7 | | Disease | Disease | KIAA0179 | | | similar to hypothetical |
| | | | | | | | | | | | | | | protein D4478 of | |
| | | | | | | | | | | | | | | S. cerevisiae. | |
| K213 | D86968_at | D86968 | PASS | 7 | 4.14 | fail | 6 | 7 | | Disease | Disease | KIAA0213 | | | Similar to Mouse TFIIi- |
| | | | | | | | | | | | | | | | associated transactivator |
| | | | | | | | | | | | | | | | factor p17 |
| | | | | | | | | | | | | | | | (GB_RO.MMU11548) |
| | | | | | | | | | | | | | | | Containing protein kinase |
| | | | | | | | | | | | | | | | motif |
| HG2175-HT2 | HG2175-HT2 | HG2175-HT | PASS | 7 | 5.43 | fail | 6 | 7 | | Disease | Disease | | | | |
| INPPL1 | L36818_at | L36818 | PASS | 7 | 21.71 | fail | 6 | 7 | | Disease | Disease | | | 51C protein | |
| GS1 | M86934_at | M86934 | PASS | 7 | 8.14 | fail | 6 | 7 | | Disease | Disease | GS2 | | | Gene from Xp22.3 which |
| | | | | | | | | | | | | | | | escapes X-inactivation. |
| | | | | | | | | | | | | | | | Function unknown. |
| MFAP1 | U04209_at | U04209 | PASS | 7 | 6.00 | fail | 6 | 7 | | Disease | Disease | | | associated microfibrillar | |
| IFNRG7A | U53830_at | U53830 | PASS | 7 | 15.29 | fail | 6 | 7 | | Disease | Disease | 1RF7 | 11 | interferon regulatory | interferon regulatory |
| | | | | | | | | | | | | | | factor 7 | factor 7 |
| GABRA1 | X14766_at | X14766 | PASS | 7 | 8.71 | fail | 6 | 7 | | Disease | Disease | GABRA1 | 5q34-q35 | gamma-aminobutyric acid | gamma-aminobutyric acid |
| | | | | | | | | | | | | | | (GABA) A receptor, | (GABA) A receptor, |
| | | | | | | | | | | | | | | alpha 1 | alpha 1 |
| CSRP1 | M76378_at | M76378 | PASS | 7 | 23.29 | fail | 5 | 7 | | Disease | Disease | CSRP1 | 1q32 | cysteine and glycine-rich | cysteine and glycine-rich |
| | | | | | | | | | | | | | | protein 1 | protein 1 |
| K146 | D63480_at | D63480 | PASS | 7 | 6.14 | fail | 5 | 7 | | Disease | Disease | KIAA0146 | | | The KIAA0146 gene |
| | | | | | | | | | | | | | | | product is novel |
| GLGF2 | HG4704-HT5 | HG4704-HT | PASS | 7 | 6.14 | fail | 5 | 7 | | Disease | Disease | | | | |
| TAL1 | M63589_at | M63589 | PASS | 7 | 13.00 | fail | 5 | 7 | | Disease | Disease | TAL1 | 1p321 | T-cell acute lymphocytic | T-cell acute lymphocytic |
| | | | | | | | | | | | | | | leukemia 1 | leukemia 1 |
| SIAHBP1 | U51586_at | U51586 | PASS | 7 | 8.29 | fail | 5 | 7 | | Disease | Disease | SiahBP1 | | siah binding protein 1 | |
| ENO2_rna1 | X51956_rna1 | X51956 | PASS | 7 | 9.43 | fail | 5 | 7 | | Disease | Disease | ENO2 | 12p13 | human gamma enolase | enolase 2, (gamma, |
| | | | | | | | | | | | | | | | neuronal) |
| K27 | D25217_at | D25217 | PASS | 7 | 16.29 | fail | 4 | 7 | | Disease | Disease | KIAA0027 | | | |
| EBI3 | L08187_at | L08187 | PASS | 7 | 13.86 | fail | 4 | 7 | | Disease | Disease | EBI3 | | cytokine receptor | |
| RETROTP | Z48633_at | Z48633 | PASS | 7 | 12.14 | fail | 4 | 7 | | Disease | Disease | | | | |
| SP2 | D28588_at | D28588 | PASS | 6 | 6.83 | fail | 6 | 6 | | Disease | Disease | SP2 | | Sp2 transcription factor | Sp2 transcription factor |
| K263_HYPY | D87452_at | D87452 | PASS | 6 | 13.33 | fail | 6 | 6 | | Disease | Disease | KIAA0263 | | | KIAA0263 gene product |
| HEPG2 | K03195_at | K03195 | PASS | 6 | 9.50 | fail | 6 | 6 | | Disease | Disease | SGLT1 | | | glucose transporter |
| | | | | | | | | | | | | | | glycoprotein | |
| IDS | L40586_at | L40586 | PASS | 6 | 6.67 | fail | 6 | 6 | | Disease | Disease | IDS | Xq27.3-q28 | iduronate 2-sulfatase | iduronate 2-sulfatase |
| | | | | | | | | | | | | | | (Hunter syndrome) | (Hunter syndrome) |
| FOSB | L49169_at | L49169 | PASS | 6 | 10.67 | fail | 6 | 6 | | Disease | Disease | GOS3 | | | GOS3 is human homolog |
| | | | | | | | | | | | | | | | of mouse FOSB gene |
| MYOIC | U14391_at | U14391 | PASS | 6 | 9.00 | fail | 6 | 6 | | Disease | Disease | MYOIC | 15g21-q22 | myocin-IC | myosin IC |
| HCCS | U36787_at | U36787 | PASS | 6 | 7.00 | fail | 6 | 6 | | Disease | Disease | | | halocytochomre c-type | putative |
| | | | | | | | | | | | | | | synthetase | |
| HA2 | X90761_at | X90761 | PASS | 6 | 8.67 | fail | 6 | 6 | | Disease | Disease | KRTHA2 | 17q12-q211 | keratin, hair, acidic, 2 | keratin, hair, acidic, 2 |
| X92493_s_at | X92493_s_at | X92493 | PASS | 6 | 5.00 | fail | 6 | 6 | | Disease | Disease | PIPSK1B | 9q13 | phosphatidylinositol-4- | phosphatidylinositol-4- |
| | | | | | | | | | | | | | | phosphate 5-kinase, | phosphate 5-kinase, |
| | | | | | | | | | | | | | | type 1, beta | type 1, beta |
| XPA | D14533_at | D14533 | PASS | 6 | 4.50 | fail | 5 | 6 | | Disease | Disease | XPA | 9 | xeroderma pigmentosum, | xeroderma pigmentosum, |
| | | | | | | | | | | | | | | complementation group A | complementation group A |
| K76 | D38548_at | D38548 | PASS | 6 | 16.33 | fail | 5 | 6 | | Disease | Disease | KIAA0076 | | | KIAA0076 gene product |
| K268_C219R | D87742_at | D87742 | PASS | 6 | 5.17 | fail | 5 | 6 | | Disease | Disease | KIAA0268 | | | Similar to Human C219- |
| | | | | | | | | | | | | | | | reactive peptide (L34688) |
| DAO | HG2280-HT2 | HG2280-HT | PASS | 6 | 15.50 | fail | 5 | 6 | | Disease | Disease | | | | |
| H2AIB | L19778_at | L19778 | PASS | 6 | 5.00 | fail | 5 | 6 | | Disease | Disease | H2AFP | | histone H2A.Ib | H2A histone family, |
| | | | | | | | | | | | | | | | member P |
| MAP4 | M64571_at | M64571 | PASS | 6 | 11.83 | fail | 5 | 6 | | Disease | Disease | MAP4 | 3p21 | microtubule-associated | microtubule-associated |
| | | | | | | | | | | | | | | protein 4 | protein 4 |
| PML | M79462_at | M79462 | PASS | 6 | 12.00 | fail | 5 | 6 | | Disease | Disease | PML | 15q22 | promycelocytic leukemia | promycelocytic leukemia |
| S78798_s_at | S78798_s_at | S78798 | PASS | 6 | 6.50 | fail | 5 | 6 | | Disease | Disease | 1-phos- | | 1-phosphatidylinositol-4- | Method: conceptual |
| | | | | | | | | | | | | phatidyl- | | phosphate 5-kinase | translation with partial |
| | | | | | | | | | | | | linositol-4- | | isoform C | peptide sequencing. This |
| | | | | | | | | | | | | phosphate 5 | | | sequence comes from |
| | | | | | | | | | | | | kinase | | | FIG. 2; PtdIns4P 5-kinase |
| | | | | | | | | | | | | isoform C, | | | isoform C |
| | | | | | | | | | | | | PtdIns4P 5- | | | |
| | | | | | | | | | | | | kinase | | | |
| | | | | | | | | | | | | isoform C | | | |
| HSD17B3 | U05659_at | U05659 | PASS | 6 | 18.17 | fail | 5 | 6 | | Disease | Disease | HSD17B3 | 9q22 | hydroxysteroid (17-beta) | hydroxysteroid (17-beta) |
| | | | | | | | | | | | | | | dehydrogenase 3 | dehydrogenase 3 |
| ASMT | U11090_at | U11090 | PASS | 6 | 11.50 | fail | 5 | 6 | | Disease | Disease | ASMT | Xpter-p22.32 | acetylserotomin N- | acetylserotonin N- |
| | | | | | | | | | | | | | and Yp11.3 | methyltransferase | methyltransferase |
| MHC2TA | U18259 at | U18259 | PASS | 6 | 5.00 | fail | 5 | 6 | | Disease | Disease | MHC2TA | Chr.16 | MHC class II transactivator | MHC class II transactivator |
| U22431_s_at | U22431_s_at | U22431 | PASS | 6 | 6.83 | fail | 5 | 6 | | Disease | Disease | HIP1A | 14q21-q24 | hypoxia-inducible factor 1, | hypoxia-inducible factor 1, |
| | | | | | | | | | | | | | | alpha subunit (basic | alpha subunit (basic |
| | | | | | | | | | | | | | | helix-loop-helix | helix-loop-helix |
| | | | | | | | | | | | | | | transcription factor | transcription factor |
| SNAP | U39412_at | U39412 | PASS | 6 | 16.50 | fail | 5 | 6 | | Disease | Disease | NAPA | | N-ethylmaleimide-sensitive | N-ethylmaleimide-sensitive |
| | | | | | | | | | | | | | | factor attachment | factor attachment |
| | | | | | | | | | | | | | | protein, alpha | protein, alpha |
| U43189_s_at | U43189_s_at | U43189 | PASS | 6 | 7.17 | fail | 5 | 6 | | Disease | Disease | NERP-1a,b | | NERF-1b | Ets transcription factor |
| CHM | U84720_at | U84720 | PASS | 6 | 1l.83 | fail | 5 | 6 | | Disease | Disease | RAE1; | | Homolog of yeast Rae1 | Homolog of yeast Rae1 |
| | | | | | | | | | | | | | | (Bharathi) mRNA- | (Bharathi) mRNA- |
| | | | | | | | | | | | | | | associated protein of | associated protein of |
| | | | | | | | | | | | | | | 41 kDa (Kraemer) | 41 kDa (Kraemer) |
| ITGA6_rna1 | X53586_rna1 | X53586 | PASS | 6 | 8.83 | fail | 5 | 6 | | Disease | Disease | ITGA6 | 2 | integrin, alpha 6 | integrin, alpha 6 |
| LY64 | D83597_at | D83597 | PASS | 6 | 11.83 | fail | 4 | 6 | | Disease | Disease | LY64 | Sq12 | RP105 | lymphocyte antigen 64 |
| | | | | | | | | | | | | | | | (mouse) homolog, |
| | | | | | | | | | | | | | | | radioprotective, 105 kD |
| MLLT3 | L13744_at | L13744 | PASS | 6 | 5.00 | fail | 4 | 6 | | Disease | Disease | AF-9 | | | |
| KPNA1 | S75295_at | S75295 | PASS | 6 | 5.67 | fail | 4 | 6 | | Disease | Disease | KPNA1 | | karopherin alpha 1 | karopherin alpha |
| | | | | | | | | | | | | | | (importin alpha 5) | (importin alpha 5) |
| ZNF133 | U09366_at | U09366 | PASS | 6 | 7.50 | fail | 4 | 6 | | Disease | Disease | ZNF169 | 9q22 | zinc finger protein 169 | zinc finger protein 169 |
| SGSH | U30894_at | U30894 | PASS | 6 | 10.50 | fail | 4 | 6 | | Disease | Disease | SGSH | 17q25.3 | N-sulfoglucosamine | N-sulfoglucosamine |
| | | | | | | | | | | | | | | sulfohydrolase | sulfohydrolase |
| | | | | | | | | | | | | | | (sulfamidase) | (sulfamidase) |
| AMT | D14686_at | D14686 | PASS | 5 | 12.20 | fail | 6 | 5 | | Disease | Disease | AMT | 3p21.2-p21.1 | aminomethyltransferase | aminomethyltransferase |
| | | | | | | | | | | | | | | (glycine cleavage system | (glycine cleave system |
| | | | | | | | | | | | | | | protein T) | protein T) |
| APT1LG1 | D38122_at | D38122 | PASS | 5 | 5.40 | fail | 6 | 5 | | Disease | Disease | TNFSF6 | 1q23 | apoptosis (APO-1) antigen | tumor necrosis factor |
| | | | | | | | | | | | | | | ligand 1 | (ligand) superfamily, |
| | | | | | | | | | | | | | | | member 6 |
| K83_CHR3 | D42046_at | D42046 | PASS | 5 | 6.60 | fail | 6 | 5 | | Disease | Disease | DNA2L | 10q21.2-22.1 | | DNA2 (DNA replication |
| | | | | | | | | | | | | | | | helicase, yeast, homolog- |
| | | | | | | | | | | | | | | | like |
| TESK1 | D50863_at | D50863 | PASS | 5 | 8.80 | fail | 6 | 5 | | Disease | Disease | TESK1 | 9p13 | TESK1 | testis-specific kianse 1 |
| K172_ANK3 | D79994_at | D79994 | PASS | 5 | 6.20 | fail | 6 | 5 | | Disease | Disease | KIAA0172 | | | similar to ankytrin of |
| | | | | | | | | | | | | | | | Chromatium vinosum. |
| HG1783-HT1 | HG1783-HT1 | HG1783-HT | PASS | 5 | 4.60 | fail | 6 | 5 | | Disease | Disease | | | | |
| HOXP12 | HG2810-HT2 | HG2810-HT | PASS | 5 | 6.00 | fail | 6 | 5 | | Disease | Disease | | | | |
| X104 | L27476_at | L27476 | PASS | 5 | 5.20 | fail | 6 | 5 | | Disease | Disease | ZO-2 | 9q13-q21 | Friedreich ataxia region | Friedreich ataxia region |
| | | | | | | | | | | | | | | gene X104 (tight junction | gene X104 (tight junction |
| | | | | | | | | | | | | | | protein ZO-2) | protein ZO-2) |
| BRE | L38616_at | L38616 | PASS | 5 | 11.00 | fail | 6 | 5 | | Disease | Disease | BRE | | brain and reproductive | brain and reproductive |
| | | | | | | | | | | | | | | organ-expressed | organ-expressed |
| | | | | | | | | | | | | | | (TNFRSF1A modulator) | (TNFRSF1A modulator) |
| ZNF8 | M29581_at | M29581 | PASS | 5 | 8.80 | fail | 6 | 5 | | Disease | Disease | ZNF8 | 20q13 | | |
| U50648_s_at | U50648_s_at | U50648 | PASS | 5 | 24.60 | fail | 6 | 5 | | Disease | Disease | PRKR | 2p22-p21 | protein kinase, interferon- | protein kinase, interferon- |
| | | | | | | | | | | | | | | inducible double stranded | inducible double stranded |
| | | | | | | | | | | | | | | RNA dependent | RNA dependent |
| SCA2 | U70323_at | U70323 | PASS | 5 | 12.20 | fail | 6 | 5 | | Disease | Disease | SCA2 | 12q24 | ataxin 2 | spinocerebellar ataxia 2 |
| | | | | | | | | | | | | | | | (olivopontocerebellar |
| | | | | | | | | | | | | | | | ataxia 2, autosomal |
| | | | | | | | | | | | | | | | dominant, ataxin 2) |
| ADARB1 | U76421_at | U76421 | PASS | 5 | 6.20 | fail | 6 | 5 | | Disease | Disease | ADARB1 | 21q22.3 | adenosine deaminase, | adenosine deaminase, |
| | | | | | | | | | | | | | | RNA-specific, B1 | RNA-specific, B1 |
| | | | | | | | | | | | | | | (homolog of rat RED1) | (homolog of rat RED1) |
| TSG101 | U82130_at | U82l30 | PASS | 5 | 7.80 | fail | 6 | 5 | | Disease | Disease | TSG101 | | tumor susceptibility protein | |
| PMM1 | U86070_at | U86070 | PASS | 5 | 12.60 | fail | 6 | 5 | | Disease | Disease | PMM1 | 22q13 | phosphomannomutase 1 | phosphomannomutase 1 |
| TST | X59434_at | X59434 | PASS | 5 | 8.80 | fail | 6 | 5 | | Disease | Disease | TST | | thiosulfate sulfurtransferase | thiosulfate sulfurtransferase |
| | | | | | | | | | | | | | | (rhodanese) | (rhodanese) |
| PFKP | D25328_at | D25328 | PASS | 5 | 8.60 | fail | 5 | 5 | | Disease | Disease | PFKP | 10p15 | platelet-type | phosphofructinase, |
| | | | | | | | | | | | | | | phosphofructokinase | platelet |
| K65_ZNF11B | D31763_at | D31763 | PASS | 5 | 7.60 | fail | 5 | 5 | | Disease | Disease | KIAA0065 | | | ha0946 protein is |
| | | | | | | | | | | | | | | | Kruppel-related |
| K188_SMP2 | D80010_at | D80010 | PASS | 5 | 5.40 | fail | 5 | 5 | | Disease | Disease | KIAA0188 | | | |
| HG2809-HT2 | H532809-HT2 | HG2809-HT | PASS | 5 | 9.80 | fail | 5 | 5 | | Disease | Disease | | | | |
| M11025_s_at | M11025_s_at | M11025 | PASS | 5 | 6.40 | fail | 5 | 5 | | Disease | Disease | ASGR2 | 17p | asialoglycoprotein | asialoglycoprotein |
| | | | | | | | | | | | | | | receptor 2 | receptor 2 |
| M19267_s_at | M19267_s_at | M19267 | PASS | 5 | 7.80 | fail | 5 | 5 | | Disease | Disease | TPM1 | 15q22.1 | tropomyosin 1 (alpha) | tropomyosin 1 (alpha) |
| TNNC1_rna1 | M37984_rna1 | M37984 | PASS | 5 | 12.80 | fail | 5 | 5 | | Disease | Disease | TnC | | slow twitch skeletal/ | putative |
| | | | | | | | | | | | | | | cardiac muscle troponin C | |
| ECGF1 | S72487_at | S72487 | PASS | 5 | 10.00 | fail | 5 | 5 | | Disease | Disease | orf1 5′ to | | | |
| | | | | | | | | | | | | PD- | | | |
| | | | | | | | | | | | | ECGF/TP | | | |
| EIF2B | U23028_at | U23028 | PASS | 5 | 8.20 | fail | 5 | 5 | | Disease | Disease | EIF2B5 | | eIF-2Bepsilon | eukaryotic translation |
| | | | | | | | | | | | | | | | initiation factor 2B, |
| | | | | | | | | | | | | | | | subunit 5 (epsilon 82 kD) |
| CBLB | U26710_at | U26710 | PASS | 5 | 6.20 | fail | 5 | 5 | | Disease | Disease | CBLB | 3q | Cas-Br-M (murine) | Cas-Br-M (murine) |
| | | | | | | | | | | | | | | ectropic retroviral | ectropic retroviral |
| | | | | | | | | | | | | | | transforming sequence b | transforming sequence b |
| ADK | U50196_at | U50196 | PASS | 5 | 5.00 | fail | 5 | 5 | | Disease | Disease | | | adenosine kinase | |
| ERVK_cds2 | U60269_cds2 | U60269 | PASS | 5 | 5.80 | fail | 5 | 5 | | Disease | Disease | | | | putative polymerase; orf |
| | | | | | | | | | | | | | | | similar to the integrase |
| | | | | | | | | | | | | | | | domain of Type A and |
| | | | | | | | | | | | | | | | Type B retroviruses and |
| | | | | | | | | | | | | | | | to class II HERVs |
| U68162₋cda | U68162_cds | U68162 | PASS | 5 | 6.40 | fail | 5 | 5 | | Disease | Disease | MPL | | thrombopoietin receptor | alternative initiation codon |
| | | | | | | | | | | | | | | | used |
| POU2AF1 | Z49194_at | Z49194 | PASS | 5 | 7.20 | fail | 5 | 5 | | Disease | Disease | POU2AF1 | 11q23.1 | oct-binding factor | POU domain, class 2, |
| | | | | | | | | | | | | | | | associating factor 1 |
| K334 | AB002332_at | AB002332 | PASS | 5 | 4.80 | fail | 4 | 5 | | Disease | Disease | CLOCK | 4q12 | clock (mouse) homolog | clock (mouse) homolog |
| K238_PERM | D87075_at | D87075 | PASS | 5 | 6.00 | fail | 4 | 5 | | Disease | Disease | KIAA0238 | | similar to Mouse yolk sac | |
| | | | | | | | | | | | | | | | permease like molecule 1 |
| | | | | | | | | | | | | | | | (U25739) |
| K258 | D87447_at | D87447 | PASS | 5 | 9.20 | fail | 4 | 5 | | Disease | Disease | KIAA0258 | | | KIAA0258 gene product |
| K279_EGFL | D87469_at | D87469 | PASS | 5 | 5.80 | fail | 4 | 5 | | Disease | Disease | EGFL2 | chr. 1 | | EGF-like-domain, |
| | | | | | | | | | | | | | | | multiple 2 |
| AMY2B | D90097_at | D90097 | PASS | 5 | 5.60 | fail | 4 | 5 | | Disease | Disease | AMY2B | 1p21 | alpha-amylase | amylase, alpha 2B; |
| | | | | | | | | | | | | | | | pancreatic |
| M34458_rna1 | M34458_rna1 | M34458 | PASS | 5 | 5.80 | fail | 4 | 5 | | Disease | Disease | | | | lamin B |
| M81182_s_at | M81182_s_at | M81182 | PASS | 5 | 5.00 | fail | 4 | 5 | | Disease | Disease | PXMP1 | 1p22-p21 | peroxisomal membrane | peroxisomal membrane |
| | | | | | | | | | | | | | | protein 1 (70 kD, Zellweger | protein 1 (70 kD, Zellweger |
| | | | | | | | | | | | | | | syndrome) | syndrome) |
| HRC1 | M91083_at | M91083 | PASS | 5 | 8.00 | fail | 4 | 5 | | Disease | Disease | C11ORF13 | 11p15.5 | HRAS1-related cluster-1 | chromsome 11 open |
| | | | | | | | | | | | | | | | reading frame 13 |
| ITGB3 | S70348_at | S70348 | PASS | 5 | 7.00 | fail | 4 | 5 | | Disease | Disease | integrin | | integrin beta 3 | This sequence comes from |
| | | | | | | | | | | | | beta 3 | | | FIG. 1a. Protein sequence |
| | | | | | | | | | | | | | | | is in conflict with the |
| | | | | | | | | | | | | | | | conceptual translation; |
| | | | | | | | | | | | | | | | mismatch (29[G-&gt;Q]) |
| PTFD | U44755_at | U44755 | PASS | 5 | 9.00 | fail | 4 | 5 | | Disease | Disease | SNAPC2 | | small nuclear RNA | small nuclear RNA |
| | | | | | | | | | | | | | | activating complex, | activating complex, |
| | | | | | | | | | | | | | | polypeptide 2, 45 kD | polypeptide 2, 45 kD |
| GCDH | U69141_at | U69141 | PASS | 5 | 6.40 | fail | 4 | 5 | | Disease | Disease | GCDH | 19p13.2 | glutaryl-Coenzyme A | glutaryl-Coenzyme A |
| | | | | | | | | | | | | | | dehydrogenase | dehydrogenase |
| UNKP | D28124_at | D28124 | PASS | 5 | 16.40 | fail | 3 | 5 | | Disease | Disease | NBL1 | 1p36.3-p36.2 | | neuroblastoma candidate |
| | | | | | | | | | | | | | | | region, suppression of |
| | | | | | | | | | | | | | | | tumorigenicity 1 |
| NCBPIP1 | D59253_at | D59253 | PASS | 5 | 5.80 | fail | 3 | 5 | | Disease | Disease | | | NCBP Interacting Protein 1 | RNA-binding protein that |
| | | | | | | | | | | | | | | has two RNP consensus | |
| | | | | | | | | | | | | | | | motifs |
| ITGB3 | HG2320-HT2 | HG2320-HT | PASS | 5 | 11.60 | fail | 3 | 5 | | Disease | Disease | | | | |
| M33684_s_at | M33684_s_at | M33684 | PASS | 5 | 6.60 | fail | 3 | 5 | | Disease | Disease | PTPN1 | | non-receptor tyrosine | |
| | | | | | | | | | | | | | | phosphatase 1 | |
| MYH10_r | U34301_r_at | U34301 | PASS | 5 | 15.40 | fail | 3 | 5 | | Disease | Disease | | | | |
| X07434_s_at | X07438_s_at | X07438 | PASS | 5 | 9.00 | fail | 3 | 5 | | Disease | Disease | | | | |
| CREB1 | X68994_at | X68994 | PASS | 5 | 5.60 | fail | 3 | 5 | | Disease | Disease | CREB | | Y protein | |
| MYL2 | J02854_at | J02854 | PASS | 5 | 26.00 | fail | 2 | 5 | | Disease | Disease | MYL2 | | myosin light chain 2 | |
| AC1 | D82070_at | D82070 | PASS | 5 | 5.00 | fail | 2 | 5 | | Disease | Disease | aC1 | | | |
| CGM7 | D90276_at | D90276 | PASS | 5 | 14.60 | fail | 2 | 5 | | Disease | Disease | CGM7 | 19q13.2 | carcinoembryonic antigen | carcinoembryonic entigen |
| | | | | | | | | | | | | | | gene family member 7 | gene family member 7 |
| HG4020-HT4 | HG4020-HT4 | HG4020-HT | PASS | 5 | 16.00 | fail | 2 | 5 | | Disease | Disease | | | | |
| PCK1 | L05144_at | L05144 | PASS | 5 | 5.60 | fail | 2 | 5 | | Disease | Disease | PCK1 | 20q13.31 | phosphoenolpyruvate | phosphoenolpyruvate |
| | | | | | | | | | | | | | | carboxykinase 1 (soluble) | carboxykinase 1 (soluble) |
| DEFA5_rna1 | M97925_rna1 | M97925 | PASS | 5 | 9.00 | fail | 2 | 5 | | Disease | Disease | DEFAS | 8pter-p21 | defensin 5 | definsin, alpha 5, Paneth |
| | | | | | | | | | | | | | | | cell-specific |
| CCNG1IP | U61836_at | U61836 | PASS | 5 | 8.80 | fail | 2 | 5 | | Disease | Disease | | | | |
| KRT3 | X82634_at | X82634 | PASS | 5 | 6.40 | fail | 2 | 5 | | Disease | Disease | KRTHA7 | 17q12-q21 | keratin, hair, acidic, 7 | keratin, hair, acidic, 7 |
| NMOR2 | J02888_at | J02888 | PASS | 7 | 12.71 | PASS | 8 | 7 | 6.38 | 1.99 | 1.99 | NMOR2 | 6pter-q12 | NAD(P)H menadione | NAD(P)H menadione |
| | | | | | | | | | | | | | | oxidoreductase 2, dioxin- | oxidoreductase 2, dioxin- |
| | | | | | | | | | | | | | | inducible | inducible |
| HG33-HT33 | HG33-HT33 | HG33-HT3 | PASS | 9 | 309.00 | PASS | 13 | 9 | 155.08 | 1.99 | 1.99 | | | | |
| RES422B | AB000460_at | AB000460 | PASS | 9 | 27.56 | PASS | 12 | 9 | 13.83 | 1.99 | 1.99 | RES4-22 | 4p16.3 | gene with multiple splice | gene with multiple splice |
| | | | | | | | | | | | | | | variants near HD locus | variants near HD locus |
| | | | | | | | | | | | | | | on 4p16.3 | on 4p16.3 |
| MIC2 | M16279_at | M16279 | PASS | 9 | 72.22 | PASS | 13 | 9 | 36.31 | 1.99 | 1.99 | MIC2 | Xp22.32; | antigen identified by | antigen identified by |
| | | | | | | | | | | | | | Yp11.3 | monocolonal antibodies | monoclonal antibodies |
| | | | | | | | | | | | | | | 12E7, F21 and O13 | 12E7, F21 and O13 |
| RB1 | L22342_at | L22342 | PASS | 9 | 27.33 | PASS | 12 | 9 | 13.75 | 1.99 | 1.99 | IFI75 | | interferon-induced protein | interferon-induced protein |
| | | | | | | | | | | | | | | 75, 52 kD | 75, 52 kD |
| HLA-DNA | M31525_at | M31525 | PASS | 6 | 13.50 | PASS | 10 | 6 | 6.80 | 1.99 | 1.99 | HLA-DNA | | | MHC HLA-DNA precursor |
| LRP1 | X79882_at | X79882 | PASS | 7 | 27.29 | PASS | 12 | 7 | 13.75 | 1.98 | 1.98 | LRP | | | lung resistance-related |
| | | | | | | | | | | | | | | | protein |
| RAGE_cds3 | U89336_cds | U89336 | PASS | 9 | 12.56 | PASS | 12 | 9 | 6.33 | 1.98 | 1.98 | HBX2 | | homeobox PBX2 gene | intron-exon boundaries |
| | | | | | | | | | | | | | | | identified by a contig of |
| | | | | | | | | | | | | | | | ESTs with GenBank |
| | | | | | | | | | | | | | | | Accession Number |
| | | | | | | | | | | | | | | | W76064, R59617, W72507 |
| J03805_s_at | J03805_s_at | J03805 | PASS | 9 | 14.33 | PASS | 13 | 9 | 7.23 | 1.98 | 1.98 | PPP2CB | 8p12-p11.2 | protein phosphatase 2 | protein phosphatase 2 |
| | | | | | | | | | | | | | | (formerly 2A), catalytic | (formerly 2A), catalytic) |
| | | | | | | | | | | | | | | subunit, beta isoform | subunit, beta isoform |
| ATOX1 | U70660_at | U70660 | PASS | 8 | 13.38 | PASS | 12 | 8 | 6.75 | 1.98 | 1.98 | ATOX1 | 5q32-q33 | ATX1 (antioxidant protein | ATX1 (antioxidant protein |
| | | | | | | | | | | | | | | 1, yeast) homolog 1 | 1, yeast) homolog 1 |
| K22 | D14664_at | D14664 | PASS | 8 | 16.50 | PASS | 12 | 8 | 8.33 | 1.98 | 1.98 | KIAA0022 | | | KIAA0022 gene product |
| SMS | Z49099_at | Z49099 | PASS | 9 | 20.44 | PASS | 12 | 9 | 10.33 | 1.98 | 1.98 | SMS | Xp22.1 | spermine synthase | spermine synthase |
| TCTEL1 | D50663_at | D50663 | PASS | 8 | 22.75 | PASS | 12 | 8 | 11.50 | 1.98 | 1.98 | TCTEL1 | | | similar to murine Tete1 |
| | | | | | | | | | | | | | | | gene product |
| CSTF3 | U15782_at | U15782 | PASS | 8 | 11.38 | PASS | 12 | 8 | 5.75 | 1.98 | 1.98 | CSTF3 | | cleavage stimulation factor | cleavage stimulation factor, |
| | | | | | | | | | | | | | | subunit 3 | 3′ pre-RNA, subunit 3, |
| | | | | | | | | | | | | | | | 77 kD |
| PL1 | M11119_at | M11119 | PASS | 7 | 15.00 | PASS | 12 | 7 | 7.58 | 1.98 | 1.98 | | | | pseudo-evn cds/pseudo |
| M20867_s_at | M20867_s_at | M20867 | PASS | 8 | 14.13 | PASS | 13 | 8 | 7.15 | 1.97 | 1.97 | GLUD1 | | | glutamate dehydrogenase |
| G6PD | M24470_at | M24470 | PASS | 9 | 1578 | PASS | 11 | 9 | 8.00 | 1.97 | 1 97 | GMPR 6p23 | | | |
| | | | | | | | | | | | | | | | (EC 1.4.1.3.) |
| K24_PTDSS | D14694_at | D14694 | PASS | 9 | 41.11 | PASS | 13 | 9 | 20.85 | 1.97 | 1.97 | KIAA0024 | | | KIAA0024 gene product |
| REQ | U94585_at | U94585 | PASS | 9 | 19.11 | PASS | 13 | 9 | 9.69 | 1.97 | 1.97 | hsReq | | requiem homolog | zinc finger; contains one |
| | | | | | | | | | | | | | | | C2H2 and two C4HC3 |
| L19493_s_at | L19493_s_at | L19493 | PASS | 7 | 9.86 | PASS | 11 | 7 | 5.00 | 1.97 | 1.97 | FMR1 | | | |
| M12959_s_at | M12959_s_at | M12959 | PASS | 9 | 137.22 | PASS | 13 | 9 | 69.62 | 1.97 | 1.97 | TCRA | | | T-cell receptor alpha- |
| | | | | | | | | | | | | | | | chain (VDJC) |
| TRSP | M86752_at | M86752 | PASS | 8 | 16.13 | PASS | 11 | 8 | 8.18 | 1.97 | 1.97 | IEF SSP | | transformation-sensitive | |
| | | | | | | | | | | | | 3521 | | protein | |
| MYCBP | D89667_at | D89667 | PASS | 9 | 114.11 | PASS | 13 | 9 | 5792 | 1.97 | 1.97 | PFDN5 | | prefoldin 5 | prefoldin 5 |
| MYL6 | HG2815-HT2 | HG2815-HT | PASS | 9 | 292.33 | PASS | 13 | 9 | 148.4 | 61.97 | 1.97 | | | | |
| GOK | U52426_at | U52426 | PASS | 7 | 14.00 | PASS | 9 | 7 | 7.11 | 1.97 | 1.97 | STIM1 | 11p15.5 | stromal interaction | stromal interaction |
| | | | | | | | | | | | | | | molecule 1 | molecule 1 |
| ALDH7 | U34252_at | U34252 | PASS | 8 | 13.63 | PASS | 13 | 8 | 6.92 | 1.97 | 1.97 | ALDH9 | 1q22-q23 | aldehyde dehydrogenase 9 | aldehyde dehydrogenase 9 |
| | | | | | | | | | | | | | | (gamma-aminobutyralde- | (gamma-aminobutyralde- |
| | | | | | | | | | | | | | | hyde dehydrogenase, | hyde dehydrogenase, |
| | | | | | | | | | | | | | | E3 isozyme) | E3 isozyme) |
| ETFB | X71129_at | X71129 | PASS | 9 | 12.00 | PASS | 10 | 9 | 6.10 | 1.97 | 1.97 | ETFB | 19q13.3 | electron-transfer- | electron-transfer- |
| | | | | | | | | | | | | | | flavoprotein, beta poly- | flavoprotein, beta poly- |
| | | | | | | | | | | | | | | peptide | peptide |
| DIPA | U63825_at | U63825 | PASS | 8 | 21.63 | PASS | 13 | 8 | 11.00 | 1.97 | 1.97 | dipA | | hepatitis delta antigen | isolated in a two hybrid |
| | | | | | | | | | | | | | | interacting protein A | screen to identify cellular |
| | | | | | | | | | | | | | | | proteins that interact with |
| | | | | | | | | | | | | | | | hepatitis delta antigen; |
| | | | | | | | | | | | | | | | similar to hepatitis delta |
| | | | | | | | | | | | | | | | antigen, and has two |
| | | | | | | | | | | | | | | | regions predicted to form |
| | | | | | | | | | | | | | | | coiled-coil protein |
| | | | | | | | | | | | | | | | interaction domains |
| NAGA | M62783_at | M62783 | PASS | 9 | 16.44 | PASS | 13 | 9 | 8.38 | 1.96 | 1.96 | NAGA | 22q13-qter | N-acetylgalactosaminidase, | N-acetylgalactosaminidase, |
| | | | | | | | | | | | | | | alpha- | alpha- |
| K317 | AB002315_at | AB002315 | PASS | 9 | 11.11 | PASS | 12 | 9 | 5.67 | 1.96 | 1.96 | KIAA0317 | | | KIAA0317 gene product |
| GLB1 | M34423_at | M34423 | PASS | 9 | 17.11 | PASS | 11 | 9 | 8.73 | 1.96 | 1.96 | GLB1 | 3p21.33 | | galactosidase, beta 1 |
| CDC34 | L22005_at | L22005 | PASS | 6 | 12.00 | PASS | 8 | 6 | 6.13 | 1.96 | 1.96 | CDC34 | 19p13.3 | ubiquitin conjugating | cell division cycle 34 |
| | | | | | | | | | | | | | | enzyme | |
| AGC1 | U16306_at | U16306 | PASS | 9 | 104.11 | PASS | 13 | 9 | 53.15 | 1.96 | 1.96 | | | chondroitin sulfate | |
| | | | | | | | | | | | | | | proteoglycan versican V0 | |
| | | | | | | | | | | | | | | splice-variant precursor | |
| | | | | | | | | | | | | | | peptide | |
| SNRPN | J04615_at | J04615 | PASS | 9 | 51.22 | PASS | 13 | 9 | 26.15 | 1.96 | 1.96 | SNRPN | 15q12 | small nuclear ribonucleo- | small nuclear ribonucleo- |
| | | | | | | | | | | | | | | protein polypeptide N | protein polypeptide N |
| ECHS1 | D13900_at | D13900 | PASS | 9 | 27.00 | PASS | 10 | 9 | 13.80 | 1.96 | 1.96 | ECHS1 | 10q26.2-q26.3 | enoyl Coenzyme A | enoyl Coenzyme A |
| | | | | | | | | | | | | | | hydratase, short chain 1, | hydratase, short chain 1, |
| | | | | | | | | | | | | | | mitochondrial | mitochondrial |
| CD14 | X13334_at | X13334 | PASS | 9 | 180.11 | PASS | 13 | 9 | 92.08 | 1.96 | 1.96 | CD14 | 5q22-q32 | CD14 antigen | CD14 antigen |
| S164 | L40392_at | L40392 | PASS | 8 | 14.00 | PASS | 12 | 8 | 7.17 | 1.95 | 1.95 | | | | ORF, putative |
| LRPAP1 | M63959_at | M63959 | PASS | 8 | 28.25 | PASS | 13 | 8 | 14.46 | 1.95 | 1.95 | LRPAP1 | 4p16.3 | low density lipoprotein- | low density lipoprotein- |
| | | | | | | | | | | | | | | related protein-associated | related protein-associated |
| | | | | | | | | | | | | | | protein 1 (alpha-2- | protein 1 (alpha-2- |
| | | | | | | | | | | | | | | macroglobulin receptor- | macroglobulin receptor- |
| | | | | | | | | | | | | | | protein 1) | protein 1) |
| TXBP151 | U33821_at | U33821 | PASS | 9 | 32.56 | PASS | 13 | 9 | 16.69 | 1.95 | 1.95 | U33821 | | TXBP151 | tax1-binding protein |
| K177_ADPR | D79999_at | D79999 | PASS | 6 | 25.50 | PASS | 13 | 6 | 13.08 | 1.95 | 1.95 | KIAA0177 | | | similar to chicken poly |
| | | | | | | | | | | | | | | | (ADP-ribose) synthase, has |
| | | | | | | | | | | | | | | | putative hydrophobic |
| | | | | | | | | | | | | | | | domain in amino acid |
| | | | | | | | | | | | | | | | positions 638-662. |
| MOZ | U47742_at | U47742 | PASS | 9 | 26.67 | PASS | 13 | 9 | 13.69 | 1.95 | 1.95 | MOZ | | monocytic leukaemia zinc | |
| | | | | | | | | | | | | | | finger protein | |
| HCG5 | X81003_at | X81003 | PASS | 9 | 16.78 | PASS | 13 | 9 | 8.62 | 1.95 | 1.95 | HCGV | 6p21.3 | | hemochromatosis candidate |
| | | | | | | | | | | | | | | | gene V |
| J04130_s_at | J04130_s_at | J04130 | PASS | 9 | 27.56 | PASS | 13 | 9 | 14.15 | 1.95 | 1.95 | SCYA4 | 17q21 | small inducible cytokine | small inducible cytokine |
| | | | | | | | | | | | | | | A4 (homologous to | A4 (homologous to |
| | | | | | | | | | | | | | | mouse (Mip-1b) | mouse (Mip-1b) |
| H3F3B | M11353_at | M11353 | PASS | 9 | 245.78 | PASS | 13 | 9 | 126.31 | 1.95 | 1.95 | H3FJ | 6p22-p21.3 | H3 histone family, | H3 histone family, |
| | | | | | | | | | | | | | | member J | member J |
| PSEN1 | L76517_at | L76517 | PASS | 9 | 18.56 | PASS | 13 | 9 | 9.54 | 1.95 | 1.95 | PSEN1 | 14q24.3 | presenilin 1 (Alzheimer | presenilin 1 (Alzheimer |
| | | | | | | | | | | | | | | disease 3) | disease 3) |
| MGST2 | U77604_at | U77604 | PASS | 8 | 19.00 | PASS | 13 | 8 | 9.77 | 1.94 | 1.94 | MGST2 | 4q28-q31 | microsomal glutathione S- | microsomal glutathione S- |
| | | | | | | | | | | | | | | transferase 2 | transferase 2 |
| MACS | D10522_at | D10522 | PASS | 8 | 13.75 | PASS | 13 | 8 | 7.08 | 1.94 | 1.94 | MACS | 6q21 | myristoylated alanine-rich | myristoylated alanine-rich |
| | | | | | | | | | | | | | | protein kinase C substrate | protein kinase C substrate |
| | | | | | | | | | | | | | | (MARCKS, 80K-L) | (MARCKS, 80K-L) |
| PSMHSN3 | D26600_at | D26600 | PASS | 9 | 46.78 | PASS | 13 | 9 | 24.08 | 1.94 | 1.94 | PSMB4 | 1q21 | proteasome (prosome, | proteasome (prosome, |
| | | | | | | | | | | | | | | macropain) subunit, beta | macropain) subunit, beta |
| | | | | | | | | | | | | | | type, 4 | type, 4 |
| MYL2 | M21812_at | M21812 | PASS | 8 | 11.38 | PASS | 7 | 8 | 5.86 | 1.94 | 1.94 | | | myosin light chain 2 | |
| SKI | U73377_at | U73377 | PASS | 9 | 11.44 | PASS | 11 | 9 | 5.91 | 1.94 | 1.94 | SHC | | p66shc | alternatively spliced |
| | | | | | | | | | | | | | | | isoform in GenBank |
| | | | | | | | | | | | | | | | Accession Number X68148 |
| CLK2 | L29218_at | L29218 | PASS | 7 | 13.71 | PASS | 11 | 7 | 7.09 | 1.93 | 1.93 | CLK2 | 1q21 | CDC-like kinase 2 | CDC-like kinase 2 |
| PPBP | M54995_at | M54995 | PASS | 9 | 251.56 | PASS | 13 | 9 | 130.31 | 1.93 | 1.93 | PPBP | 4q12-q13 | pro-platelet basic protein | pro-platelet basic protein |
| | | | | | | | | | | | | | | (includes platelet basic | (includes platelet basic |
| | | | | | | | | | | | | | | protein, beta-thrombo- | protein, beta-thrombo- |
| | | | | | | | | | | | | | | globulin, connective | globulin, connective |
| | | | | | | | | | | | | | | tissue-activating peptide | tissue-activating peptide |
| | | | | | | | | | | | | | | III, neu | III, neutrophil-activating |
| | | | | | | | | | | | | | | | peptide-2) |
| SDHA | L21936_at | L21936 | PASS | 9 | 23.44 | PASS | 13 | 9 | 12.15 | 1.93 | 1.93 | SDHA | 5p15 | succinate dehydrogenase | succinate dehydrogenase |
| | | | | | | | | | | | | | | complex, subunit A, | complex subunit A, |
| | | | | | | | | | | | | | | flavoprotein (Fp) | flavorprotein (Fp) |
| UBA52 | S79522_at | S79522 | PASS | 9 | 286.56 | PASS | 13 | 9 | 148.69 | 1.93 | 1.93 | RPS27A | 2 | ribosomal protein S27a | ribosomal protein S27a |
| SARS | X91257_at | X91257 | PASS | 9 | 38.22 | PASS | 13 | 9 | 19.85 | 1.93 | 1.93 | serS | | seryl-tRNA synthetase | |
| GPS2 | U28963_at | U28963 | PASS | 8 | 13.63 | PASS | 13 | 8 | 7.08 | 1.93 | 1.93 | GPS2 | | G protein pathway | G protein pathway |
| | | | | | | | | | | | | | | suppressor 2 | suppressor 2 |
| ELANH2 | M93056_at | M93056 | PASS | 8 | 16.13 | PASS | 13 | 8 | 8.38 | 1.92 | 1.92 | ELANH2 | 6p25 | | protease inhibitor 2 (anti- |
| | | | | | | | | | | | | | | | elastase), monocyte/ |
| | | | | | | | | | | | | | | | neutrophil |
| NT5 | D38524_at | D38524 | PASS | 8 | 10.00 | PASS | 10 | 8 | 5.20 | 1.92 | 1.92 | | | 5′-nucleotidase | putative |
| ATP5O | S77356_at | S77356 | PASS | 9 | 21.00 | PASS | 13 | 9 | 10.92 | 1.92 | 1.92 | transcript | | | oligomycin sensitivty |
| | | | | | | | | | | | | ch21 | | | conferral protein oscp |
| | | | | | | | | | | | | | | | homolog; This sequence |
| | | | | | | | | | | | | | | | from FIG. 3. Protein |
| | | | | | | | | | | | | | | | sequence is in conflict |
| | | | | | | | | | | | | | | | with the conceptual |
| | | | | | | | | | | | | | | | translation, insertious |
| | | | | | | | | | | | | | | | (7-9, missing Y) |
| K58 | D31767_at | D31767 | PASS | 9 | 108.22 | PASS | 13 | 9 | 56.31 | 1.92 | 1.92 | KIAA0058 | | | KIAA0058 gene product |
| YY1 | M77698_at | M77698 | PASS | 9 | 13.44 | PASS | 9 | 9 | 7.00 | 1.92 | 1.92 | YY1 | 14q | YY1 transcription factor | YY1 transcripton factor |
| POLR2 | U37690_at | U37690 | PASS | 9 | 51.56 | PASS | 13 | 9 | 26.85 | 1.92 | 1.92 | hsRPB10 | | RNA polymerase II subunit | |
| CR3p21IGS | L13434_at | L13434 | PASS | 6 | 12.00 | PASS | 8 | 6 | 6.25 | 1.92 | 1.92 | | | | |
| MEA | HG1869-HT1 | HG1869-HT | PASS | 9 | 19.33 | PASS | 13 | 9 | 10.08 | 1.92 | 1.92 | | | | |
| PSMC3 | M34079_at | M34079 | PASS | 6 | 13.17 | PASS | 8 | 6 | 6.88 | 1.92 | 1.92 | PSMC3 | 11p12-p13 | proteasome (prosome, | proteasome (prosome, |
| | | | | | | | | | | | | | | macropain) 26S subunit, | macropain) 26S subunit,) |
| | | | | | | | | | | | | | | ATPase, 3 | ATPase, 3 |
| CTGB43A | L10378_at | L10378 | PASS | 9 | 18.22 | PASS | 13 | 9 | 9.54 | 1.91 | 1.91 | | | | |
| K84 | D42043_at | D42043 | PASS | 9 | 26.00 | PASS | 13 | 9 | 13.62 | 1.91 | 1.91 | K1AA0084 | | | The ha2022 gene product is |
| | | | | | | | | | | | | | | | novel. |
| UB_rna1 | U49869_rna1 | U49869 | PASS | 9 | 329.78 | PASS | 13 | 9 | 173.00 | 1.91 | 1.91 | | | ubiquitin | |
| CYBA | M21186_at | M21186 | PASS | 9 | 221.67 | PASS | 13 | 9 | 116.38 | 1.90 | 1.90 | CYBA | 16q24 | cytochrome b-245 alpha | cytochrome b-245 alpha |
| | | | | | | | | | | | | | | polypeptide | polpeptide |
| PGAM1 | J04173_at | J04173 | PASS | 9 | 72.56 | PASS | 13 | 9 | 38.15 | 1.90 | 1.90 | PGAM1 | 10q25.3 | phosphoglycerate mutase 1 | phosphoglycerate mutase 1 |
| | | | | | | | | | | | | | | (brain) | (brain) |
| M6PR_rna1 | X56253_rna1 | X56253 | PASS | 9 | 46.33 | PASS | 13 | 9 | 24.38 | 1.90 | 1.90 | | | | |
| ITGB5 | J05633_at | J05633 | PASS | 7 | 9.29 | PASS | 9 | 7 | 4.89 | 1.90 | 1.90 | ITGB5 | | integrin, beta 5 | integrin, beta 5 |
| U68105_s_at | U68105 s_at | U68105 | PASS | 9 | 97.56 | PASS | 13 | 9 | 51.38 | 1.90 | 1.90 | | | | |
| ADAR | U10439_at | U10439 | PASS | 9 | 34.56 | PASS | 13 | 9 | 18.23 | 1.90 | 1.90 | AI3AR | 1q21.1-q21.2 | adenosine deaminase, | adenosine deaminase, |
| | | | | | | | | | | | | | | RNA-specific | RNA-specific |
| TP53BP2 | U58334_at | U58334 | PASS | 6 | 12.00 | PASS | 12 | 6 | 6.33 | 1.89 | 1.89 | TP53BP2 | 1q42.1 | Bbp/53BP2 | tumor protein p53- |
| | | | | | | | | | | | | | | | binding protein, 2 |
| K135_PIM1 | D50925_at | D50925 | PASS | 6 | 12.00 | PASS | 9 | 6 | 6.33 | 1.89 | 1.89 | KIAA0135 | | The KIAA0135 gene is | |
| | | | | | | | | | | | | | | | related to pim-1 oncogene. |
| EWSR1 | X71428_at | X71428 | PASS | 9 | 72.00 | PASS | 13 | 9 | 38.00 | 1.89 | 1.89 | PUS | 16p11.2 | FUS gycline rich protein | fusion, derived from |
| | | | | | | | | | | | | | | | y(12;16) malignant |
| | | | | | | | | | | | | | | | liposarcoma |
| PSMD7 | U70735_at | U70735 | PASS | 9 | 15.44 | PASS | 13 | 9 | 8.15 | 1.89 | 1.89 | | | 34 kDa Mov34 homolog | similar to Mov34 |
| ZAP112 | L40399_at | L40399 | PASS | 6 | 26.50 | PASS | 10 | 6 | 14.00 | 1.89 | 1.89 | | | | ORF; putative |
| ALOX5 | J03600_at | J03600 | PASS | 9 | 29.33 | PASS | 12 | 9 | 15.50 | 1.89 | 1.89 | ALOX5 | 10g11.2 | arachidonate 5-lipoxy- | arachidonate 5-lipoxy- |
| | | | | | | | | | | | | | | genase | genase |
| MEN1_rna2 | U93237_rna2 | U93237 | PASS | 6 | 11.67 | PASS | 12 | 6 | 6.17 | 1.89 | 1.89 | MENI | 11q13 | multiple endocrine | multiple endocrine |
| | | | | | | | | | | | | | | neoplasia I | neoplasia I |
| K262 | D87451_at | D87451 | PASS | 9 | 54.89 | PASS | 13 | 9 | 29.08 | 1.89 | 1.89 | KIAA0262 | | | KIAA0262 gene product |
| X55448_cds1 | X55448_cds1 | X55448 | PASS | 9 | 27.44 | PASS | 13 | 9 | 14.54 | 1.89 | 1.89 | G6PD | | glucose-6-phosphate | |
| | | | | | | | | | | | | | | dehydrogenase | |
| E_23745 | U79260_at | U79260 | PASS | 6 | 12.33 | PASS | 11 | 6 | 6.55 | 1.88 | 1.88 | | | | similar to human oligo- |
| | | | | | | | | | | | | | | | dendrocyte myelin glyco- |
| | | | | | | | | | | | | | | | protein encoded |
| | | | | | | | | | | | | | | | by GenBank Accession |
| | | | | | | | | | | | | | | | Number L05367 |
| CBOG53955 | D87119_at | D87119 | PASS | 8 | 19.75 | PASS | 12 | 8 | 10.50 | 1.88 | 1.88 | | | GS3955 | |
| BLVRA | U34877_at | U34877 | PASS | 8 | 14.88 | PASS | 12 | 8 | 7.92 | 1.88 | 1.88 | BLVRA | 7p14-cen | biliverdin reductase A | biliverdin reductase A |
| HEXB | M23294_at | M23294 | PASS | 6 | 20.67 | PASS | 12 | 6 | 11.00 | 1.88 | 1.88 | HEXB | 5q13 | hexosaminidase B (beta | hexosaminidase B (beta |
| | | | | | | | | | | | | | | polypeptide) | polypeptide) |
| AC002045_x | AC002045_x | AC002045 | PASS | 9 | 25.00 | PASS | 13 | 9 | 13.31 | 1.88 | 1.88 | A-589H1.1 | | Unknown protein product | |
| | | | | | | | | | | | | | | | CIT987SK-A-589H1_1 |
| | | | | | | | | | | | | | | | splice form 1 |
| NOL1 | HG1116-HT1 | HG1116-HT | PASS | 7 | 12.86 | PASS | 13 | 7 | 6.85 | 1.88 | 1.88 | | | | |
| HG2090-HT2 | HG2090-HT2 | HG2090-HT | PASS | 9 | 16.89 | PASS | 11 | 9 | 9.00 | 1.88 | 1.88 | | | | |
| NDUFA4 | U94586_at | U94586 | PASS | 9 | 32.44 | PASS | 13 | 9 | 17.31 | 1.87 | 1.87 | NDUFA4 | | NADH dehydrogenase | NADH dehydrogenase |
| | | | | | | | | | | | | | | (ubiquinone) 1 alpha | (ubiquinone) 1 alpha |
| | | | | | | | | | | | | | | subcomplex, 4 (9 kD, | subcomplex, 4 (9 kD, |
| | | | | | | | | | | | | | | MLRQ) | MLRQ) |
| K59_DMT | D31883_at | D31883 | PASS | 9 | 45.56 | PASS | 13 | 9 | 24.31 | 1.87 | 1.87 | LIMAB1 | 10q25 | LIM actin binding | LIM actin binding |
| | | | | | | | | | | | | | | (limatin) | (limatin) |
| 3P25MP | L09260_at | L09260 | PASS | 9 | 16.11 | PASS | 13 | 9 | 8.62 | 1.87 | 1.87 | | | | |
| YWHAZ | M86400_at | M86400 | PASS | 9 | 116.22 | PASS | 13 | 9 | 62.15 | 1.87 | 1.87 | YWHAZ | 2p25.2-p25.1 | tyrosine 3-monoxy- | tyrosine 3-monoxy- |
| | | | | | | | | | | | | | | genase/tryptophan 5- | genase/tryptophan 5- |
| | | | | | | | | | | | | | | monoxygenase activation | monoxygenase activation |
| | | | | | | | | | | | | | | protein, zeta polypeptide | protein, zeta polypeptide |
| Z74792_s_at | Z74792 s_at | Z74792 | PASS | 5 | 10.80 | PASS | 9 | 5 | 5.78 | 1.87 | 1.87 | | | CCAAT transcription | |
| | | | | | | | | | | | | | | binding factor, gamma | |
| | | | | | | | | | | | | | | subunit | |
| HBG2_rna1 | M91036_rna1 | M91036 | PASS | 9 | 60.56 | PASS | 12 | 9 | 32.42 | 1.87 | 1.87 | HBG1 | 11p15.5 | hemoglobin, gamma A | hemoglobin, gamma A |
| COX6B_cds | AC002115_c | AC002115 | PASS | 9 | 114.11 | PASS | 13 | 9 | 61.23 | 1.86 | 1.86 | COX6B | | F25451_2 | hypothetical 36.5 kDa |
| | | | | | | | | | | | | | | | protein most similar to |
| | | | | | | | | | | | | | | | ssRNA binding proteins; |
| | | | | | | | | | | | | | | | BLASTX similarity to |
| | | | | | | | | | | | | | | | (Y07952) ssRNA-binding |
| | | | | | | | | | | | | | | | protein [Dictostellum |
| | | | | | | | | | | | | | | | discoideum] (52%) |
| | | | | | | | | | | | | | | | within RNP domains; and |
| | | | | | | | | | | | | | | | to (Z70043) hypothetical |
| | | | | | | | | | | | | | | | 24.4 kD protein C22E12.02 |
| | | | | | | | | | | | | | | | in chromosome I |
| | | | | | | | | | | | | | | | [Schizosaccharomyces |
| | | | | | | | | | | | | | | | pombe] |
| PSMHSC10 | D26598_at | D26598 | PASS | 9 | 45.56 | PASS | 13 | 9 | 24.46 | 1.86 | 1.86 | PSMB3 | 2q35 | proteasome (prosome, | proteasome (prosome, |
| | | | | | | | | | | | | | | macropain) subunit, beta | macropain) subunit, beta |
| | | | | | | | | | | | | | | type, 3 | type, 3 |
| M14328_s_at | M14328_s_at | M14328 | PASS | 9 | 173.22 | PASS | 13 | 9 | 93.15 | 1.86 | 1.86 | ENO1 | 1p36.2-p36.3 | enolase 1, (alpha) | enolase 1, (alpha) |
| CAPN2 | M23254_at | M23254 | PASS | 9 | 63.33 | PASS | 13 | 9 | 34.08 | 1.86 | 1.86 | CAPN2 | 1 | calpain, large | calpain, large |
| | | | | | | | | | | | | | | polypeptide L2 | polypeptide L2 |
| HG110-IIT11 | HG110-HT11 | HG110-HT | PASS | 9 | 26.44 | PASS | 13 | 9 | 14.23 | 1.86 | 1.86 | | | | |
| U45448_s_at | U45448_s_at | U45448 | PASS | 8 | 13.63 | PASS | 12 | 8 | 7.33 | 1.86 | 1.86 | P2RX1 | 17p | purinergic receptor P2X, | purinergic receptor P2X, |
| | | | | | | | | | | | | | | ligand-gated ion channel 1 | ligand-gated ion channel 1 |
| K275_SPOC1 | D87465_at | D87465 | PASS | 9 | 50.44 | PASS | 13 | 9 | 27.15 | 1.86 | 1.86 | KIAA0275 | | | KIAA0275 gene product |
| NCL_rna1 | M60858_rna1 | M60858 | PASS | 9 | 73.00 | PASS | 13 | 9 | 39.31 | 1.86 | 1.86 | NCL | 2g12-qter | nucleolin | nucleolin |
| IRATHL60N | U09196_at | U09196 | PASS | 9 | 27.00 | PASS | 13 | 9 | 14.54 | 1.86 | 1.86 | | | | |
| CPBP | U44975_at | U44975 | PASS | 8 | 13.00 | PASS | 11 | 8 | 7.00 | 1.86 | 1.86 | COPEB | 10p15 | core promoter element | core promoter element |
| | | | | | | | | | | | | | | binding protein | binding protein |
| ATP5G1 | D13118_at | D13118 | PASS | 9 | 37.44 | PASS | 12 | 9 | 20.17 | 1.86 | 1.86 | ATP5G3 | 2 | ATP synthase, H+ | ATP synthase, H+ |
| | | | | | | | | | | | | | | transporting, mitochondrial | transporting, mitochondrial |
| | | | | | | | | | | | | | | FO complex, subunit c | FO complex, subunit c |
| | | | | | | | | | | | | | | (subunit 9) isoform 3 | (subunit 9) isoform 3 |
| EIF3 | U36764_at | U36764 | PASS | 9 | 25.33 | PASS | 13 | 9 | 13.69 | 1.85 | 1.85 | EIF352 | 1p34.1 | eukaryotic translation | eukaryotic translation |
| | | | | | | | | | | | | | | initiation factor 3, | initiation factor 3, |
| | | | | | | | | | | | | | | subunit 2 (beta, 36 kD) | subunit 2 (beta, 36 kD) |
| XBP1 | M31627_at | M31627 | PASS | 9 | 33.00 | PASS | 13 | 9 | 17.85 | 1.85 | 1.85 | XBP1 | 22 | | X-box binding protein 1 |
| HLA-A_f | HG3597-HT3 | HG3597-HT | PASS | 9 | 182.11 | PASS | 13 | 9 | 98.62 | 1.85 | 185 | | | | |
| K77 | D38521 _at | D38521 | PASS | 9 | 11.67 | PASS | 12 | 9 | 6.33 | 1.84 | 1.84 | KIAA0077 | | | The ha0919 gene product is |
| | | | | | | | | | | | | | | | novel. |
| KPNB1 | L38951_at | L38951 | PASS | 7 | 15.86 | PASS | 13 | 7 | 8.62 | 1.84 | 1.84 | KPNB1 | | karopherin (importin) | karopherin (importin) |
| | | | | | | | | | | | | | | beta 1 | beta 1 |
| NDP52 | U22897_at | U22897 | PASS | 9 | 14.22 | PASS | 11 | 9 | 7.73 | 1.84 | 1.84 | ndp52 | | NDP52 | nuclear domain 10 protein |
| | | | | | | | | | | | | | | | as characterized by a |
| | | | | | | | | | | | | | | | monoclonal antibody |
| | | | | | | | | | | | | | | | recognizing the encoded |
| | | | | | | | | | | | | | | | protein |
| ARF1 | M84332_at | M84332 | PASS | 9 | 115.56 | PASS | 13 | 9 | 62.85 | 1.84 | 1.84 | ARF1 | 1q42 | ADP-ribosylation factor 1 | ADP ribosyltation factor 1 |
| DAD1 | D15057_at | D15057 | PASS | 9 | 20.22 | PASS | 13 | 9 | 11.00 | 1.84 | 1.84 | DAD1 | 14q11-q12 | defender against cell | defender against cell |
| | | | | | | | | | | | | | | death 1 | death 1 |
| PPIA_rna1 | X52851_rna1 | X52851 | PASS | 9 | 139.56 | PASS | 13 | 9 | 75.92 | 1.84 | 1.84 | | | peptidylprolyl isomerase | |
| PPP2RB56A | L42373_at | L42373 | PASS | 9 | 17.78 | PASS | 13 | 9 | 9.69 | 1.83 | 1.83 | PPP2R5A | 1q41 | protein phosphatase 2A | protein phosphatase 2, |
| | | | | | | | | | | | | | | B56-alpha | regulatory subunit B |
| | | | | | | | | | | | | | | | (B56), alpha isoform |
| EIF4C | L18960_at | L18960 | PASS | 9 | 11.00 | PASS | 11 | 9 | 6.00 | 1.83 | 1.83 | EIF1AY | Y chr. | eukaryotic translation | eukaryotic translation |
| | | | | | | | | | | | | | | initiation factor 1A, | initiation factor 1A, |
| | | | | | | | | | | | | | | Y chromosome | Y chromosome |
| GCH1 | U19523_at | U19523 | PASS | 7 | 10.86 | PASS | 13 | 7 | 5.92 | 1.83 | 1.83 | GCH1 | 14q22.1-q22.2 | GTP cyclohydrolase 1 | GTP cyclohydrolase 1 |
| | | | | | | | | | | | | | | (dopa-responsive dystomia) | (dopa-responsive dystomia) |
| CD37 | X14046_at | X14046 | PASS | 9 | 74.67 | PASS | 13 | 9 | 40.77 | 1.83 | 1.83 | CD37 | 19q13-q13.4 | CD37 antigen | CD37 antigen |
| K240 | D87077 at | D87077 | PASS | 6 | 9.33 | PASS | 10 | 6 | 5.10 | 1.83 | 1.83 | KIAA0240 | | | |
| M24485_s_at | M24485_s_at | M24485 | PASS | 9 | 80.33 | PASS | 13 | 9 | 43.92 | 1.83 | 1.83 | GSTP1 | 11q13 | glutathione S-transferase pi | glutathione S-transferase pi |
| HSPD1 | M22382_at | M22382 | PASS | 9 | 31.22 | PASS | 13 | 9 | 17.08 | 1.83 | 1.83 | HSPD1 | | heat shock 60 kD protein 1 | heat shock 60 kD protein 1 |
| | | | | | | | | | | | | | | (chaperonin) | (chaperonin) |
| D26156_s_at | D26156_s_at | D26156 | PASS | 8 | 13.63 | PASS | 13 | 8 | 7.46 | 1.83 | 1.83 | SMARCA4 | | SWI/SNF related, matrix | SWI/SNF related, matrix |
| | | | | | | | | | | | | | | associated, actin dependent | associated, actin dependent |
| | | | | | | | | | | | | | | regulator of chromatin, | regulator of chromatin, |
| | | | | | | | | | | | | | | subfamily a, member 4 | subfamily a, member 4 |
| HLA-C_f | HG658-HT65 | HG658-HT | PASS | 9 | 320.78 | PASS | 13 | 9 | 175.77 | 1.82 | 1.82 | | | | |
| HLA-E_f | HG2917-HT3 | HG2917-HT | PASS | 9 | 248.00 | PASS | 13 | 9 | 136.00 | 1.82 | 1.82 | | | | |
| K34_CLTCL | D21260_at | D21260 | PASS | 9 | 23.00 | PASS | 13 | 9 | 12.62 | 1.82 | 1.82 | CLTCL2 | | clathrin, heavy poly- | clathrin, heavy poly- |
| | | | | | | | | | | | | | | peptide-like 2 | peptide-like 2 |
| PRKCD | D10495_at | D10495 | PASS | 9 | 30.22 | PASS | 12 | 9 | 16.58 | 1.82 | 1.82 | 35550 | | protein kinase C delta-type | |
| M37238_s_at | M37238_s_at | M37238 | PASS | 9 | 11.33 | PASS | 9 | 9 | 6.22 | 1.82 | 1.82 | PLCG2 | 16q24.1 | phospholipase C, gamma 2 | phospholipase C, gamma 2 |
| | | | | | | | | | | | | | | (phosphatidylinositol- | (phosphatidylinositol- |
| | | | | | | | | | | | | | | specific) | specific) |
| RER1 | AJ001421_at | AJ001421 | PASS | 9 | 33.89 | PASS | 13 | 9 | 18.62 | 1.82 | 1.82 | | | Rer1 protein | |
| PPR1A | HG1614-HT1 | HG1614-HT | PASS | 9 | 106.56 | PASS | 13 | 9 | 58.62 | 1.82 | 1.82 | | | | |
| PPP2R4 | U37352_at | U37352 | PASS | 7 | 12.57 | PASS | 13 | 7 | 6.92 | 1.82 | 1.82 | PPP2R4 | 9q34 | protein phosphatase 2A, | protein phosphatase 2A, |
| | | | | | | | | | | | | | | regulatory subunit B′ | regulatory subunit B′ |
| | | | | | | | | | | | | | | (PR 53) | (PR 53) |
| HUNC18B2 | AB002559_at | AB002559 | PASS | 8 | 21.63 | PASS | 11 | 8 | 11.91 | 1.82 | 1.82 | | | hunc18b2 | putative alternatively |
| | | | | | | | | | | | | | | | spliced form of |
| | | | | | | | | | | | | | | | gbIU63533IHSU63533 |
| J03077_s_at | J03077_s_at | J03077 | PASS | 9 | 339.89 | PASS | 13 | 9 | 187.54 | 1.81 | 1.81 | PSAP | 10q21-q22 | | prosposm (variant Gaucher |
| | | | | | | | | | | | | | | | disease and variant meta- |
| | | | | | | | | | | | | | | | chromatic leukodystrophy) |
| ARRB2 | HG2059-HT2 | HG2059-HT | PASS | 9 | 24.67 | PASS | 13 | 9 | 13.62 | 1.81 | 1.81 | | | | |
| E_23652 | U90911_at | U90911 | PASS | 9 | 13.00 | PASS | 11 | 9 | 7.18 | 1.81 | 1.81 | | | | |
| HG1595-HT4 | HG1595-HT4 | HG1595-HT | PASS | 8 | 26.00 | PASS | 13 | 8 | 14.38 | 1.81 | 1.81 | | | | |
| CSNK1D | U29171_at | U29171 | PASS | 9 | 18.22 | PASS | 12 | 9 | 10.08 | 1.81 | 1.81 | CSNK1D | 17q25 | casein kinase 1, delta | casein kinase 1, delta |
| RPS3A | M84711_at | M84711 | PASS | 9 | 298.78 | PASS | 13 | 9 | 165.62 | 1.80 | 1.80 | RPS3A | 4q31.2-q31.3 | ribosomal protein S3A | ribosomal protein S3A |
| GLUL | X59834_at | X59834 | PASS | 9 | 23.00 | PASS | 12 | 9 | 12.75 | 1.80 | 1.80 | GLUL | 1q31 | glutamate-ammonia ligase | glutamate-ammonia ligase |
| | | | | | | | | | | | | | | (glutamine synthase) | (glutamine synthase) |
| IL4R | X52425_at | X52425 | PASS | 9 | 26.22 | PASS | 11 | 9 | 14.55 | 1.80 | 1.80 | IL4R | 16p11.2-p12.1 | interleukin 4 receptor | interleukin 4 receptor |
| CSTB_rna1 | U46692_rna1 | U46692 | PASS | 9 | 47.22 | PASS | 13 | 9 | 26.23 | 1.80 | 1.80 | CSTB | 21q22.3 | cystatin B (stefin B) | systatin B (stefin B) |
| M22348_s_at | M22348_s_at | M22348 | PASS | 8 | 11.38 | PASS | 9 | 8 | 6.33 | 1.80 | 1.80 | UQBP | | | ubiquinone-binding protein |
| | | | | | | | | | | | | | | | precursor |
| RP517 | M18000_at | M18000 | PASS | 9 | 464.56 | PASS | 13 | 9 | 258.69 | 1.80 | 1.80 | RPS17 | 11pter-p13 or | ribosomal protein S17 | ribosomal protein S17 |
| | | | | | | | | | | | | | 15q | 15q | |
| RPLP0 | M17885_at | M17885 | PASS | 9 | 436.11 | PASS | 13 | 9 | 243.00 | 1.79 | 1.79 | RPLP0 | 12 | ribosomal protein, large, P0 | ribosomal protein, large, P0 |
| YRS28 | D14530_at | D14530 | PASS | 9 | 367.89 | PASS | 13 | 9 | 205.31 | 1.79 | 1.79 | RPS23 | 5q | ribosomal protein S23 | ribosomal protein S23 |
| CBFB | L20298_at | L20298 | PASS | 9 | 21.78 | PASS | 13 | 9 | 12.15 | 1.79 | 1.79 | CBFB | 16q22.1 | transcription factor | core-binding factor, |
| | | | | | | | | | | | | | | beta subunit | |
| CANX | L10284_at | L10284 | PASS | 9 | 41.89 | PASS | 13 | 9 | 23.38 | 1.79 | 1.79 | CANX | 5q35 | calnexin | calnexin |
| HLARK | U89505_at | U89505 | PASS | 8 | 17.75 | PASS | 13 | 8 | 9.92 | 1.79 | 1.79 | RBM4 | 11q13 | RNA binding motif protein | RNA binding motif protein |
| | | | | | | | | | | | | | | 4 | 4 |
| ZFP | U69645_at | U69645 | PASS | 8 | 10.13 | PASS | 12 | 8 | 5.67 | 1.79 | 1.79 | | | zinc finger protein | C2H2 type zinc finger |
| SU11 | L26247_at | L26247 | PASS | 9 | 177.22 | PASS | 13 | 9 | 99.38 | 1.78 | 1.78 | sui1isol | | isolog of yeast sui1 and | |
| | | | | | | | | | | | | | | | rice gos2; putative |
| VDAC1 | L06132_at | L06132 | PASS | 8 | 15.50 | PASS | 13 | 8 | 8.69 | 1.78 | 1.78 | VDAC1 | 5q31 | voltage-dependent anion | voltage-dependent anion |
| | | | | | | | | | | | | | | channel 1 | channel 1 |
| L12711_s_at | L12711_s_at | L12711 | PASS | 9 | 67.89 | PASS | 13 | 9 | 38.08 | 1.78 | 1.78 | TKT | 3p14.3 | transketolase | transketolase (Wenncke- |
| | | | | | | | | | | | | | | | Korsakoff syndrome) |
| EIF4AI | D13748_at | D13748 | PASS | 9 | 107.22 | PASS | 13 | 9 | 60.15 | 1.78 | 1.78 | EIF4A1 | 17p13 | eukaryotic translation | eukaryotic translation |
| | | | | | | | | | | | | | | initiation factor 4A, | initiation factor 4A, |
| | | | | | | | | | | | | | | isoform 1 | isoform 1 |
| STAT13 | AFFX-HUM1 | AFFX-HUM | PASS | 9 | 23.44 | PASS | 13 | 9 | 13.15 | 1.78 | 1.78 | | | | |
| POLR2G_rna1 | U52427_rna1 | U52427 | PASS | 9 | 29.56 | PASS | 12 | 9 | 16.58 | 1.78 | 1.78 | POLR2G | 11q13.1 | polymerase (RNA) II | polymerase (RNA) II |
| | | | | | | | | | | | | | | (DNA directed) polypeptide | (DNA directed) polypeptide |
| | | | | | | | | | | | | | | G | G |
| M61832_s_at | M61832_s_at | M61832 | PASS | 7 | 10.29 | PASS | 9 | 7 | 5.78 | 1.78 | 1.78 | AHCY | 20cen-q13.1 | S-adenosylhomocysteine | S-adosylhomocysteine |
| | | | | | | | | | | | | | | hydrolase | hydrolase |
| ADPRT | J03473_at | J03473 | PASS | 9 | 17.11 | PASS | 13 | 9 | 9.62 | 1.78 | 1.78 | ADPRT | 1q41-q42 | ADP-ribosyltransferase | ADP-ribosyltransferase |
| | | | | | | | | | | | | | | (NAD+; poly (ADP- | (NAD+; poly (ADP- |
| | | | | | | | | | | | | | | ribose-polymerase) | ribose-polymerase) |
| HSPA9 | L11066_at | L11066 | PASS | 9 | 28.33 | PASS | 13 | 9 | 15.92 | 1.78 | 1.78 | | | | |
| SMC1_xpt2 | Z97054_xpt2 | Z97054 | PASS | 6 | 10.00 | PASS | 8 | 6 | 5.63 | 1.78 | 1.78 | | | KIAA0178 | match D80000; similar to |
| | | | | | | | | | | | | | | | mitosis-specific |
| | | | | | | | | | | | | | | | chromosome; segregation |
| | | | | | | | | | | | | | | | protein SMC1 of S. |
| | | | | | | | | | | | | | | | cerevisiae |
| RPL34 | L38941_at | L38941 | PASS | 9 | 432.56 | PASS | 13 | 9 | 243.62 | 1.78 | 1.78 | RPL34 | 4 or 17 | ribosomal protein L34 | ribosomal protein L34 |
| HLA-E_f | HG2915-HT3 | HG2915-HT | PASS | 9 | 245.00 | PASS | 13 | 9 | 138.15 | 1.77 | 1.77 | | | | |
| AMPHL | U68485_at | U68485 | PASS | 5 | 29.40 | PASS | 12 | 5 | 16.58 | 1.77 | 1.77 | AMPHL | 2q14 | amphiphysin-like | amphiphysin-like |
| MGAT1 | M55621_at | M55621 | PASS | 8 | 18.13 | PASS | 13 | 8 | 10.23 | 1.77 | 1.77 | MGAT1 | 5 | mannosyl (alpha-1,3-)- | mannosyl (alpha-1,3-)- |
| | | | | | | | | | | | | | | glycoprotein beta-1,2-N- | glycoprotein beta-1,2-N- |
| | | | | | | | | | | | | | | acetylglucosanimyl- | acetylglucosaminyl- |
| | | | | | | | | | | | | | | transferase | transferase |
| PDHA1 | D90084_at | D90084 | PASS | 8 | 10.63 | PASS | 9 | 8 | 6.00 | 1.77 | 1.77 | PDHA1 | Xp22.1 | pyruvate dehydrogenase | pyruvate dehydrogenase |
| | | | | | | | | | | | | | | (lipoamide) alpha 1 | (lipoamide) alpha 1 |
| COX4 | U90915_at | U90915 | PASS | 9 | 104.44 | PASS | 13 | 9 | 59.00 | 1.77 | 1.77 | COX4 | 16q22-qter | cytochrome c oxidase | cytochrome c oxidase |
| | | | | | | | | | | | | | | subunit IV | subunit IV |
| COPINE1 | U83246 at | U83246 | PASS | 9 | 49.11 | PASS | 13 | 9 | 27.77 | 1.77 | 1.77 | CPNE1 | | copine 1 | copine 1 |
| EDG2 | U11861_at | U11861 | PASS | 9 | 38.89 | PASS | 13 | 9 | 22.00 | 1.77 | 1.77 | G10 | | maternal G10 transcript | maternal G10 transcript |
| K96_PK | D43636_at | D43636 | PASS | 9 | 15.89 | PASS | 13 | 9 | 9.00 | 1.77 | 1.77 | KIAA0096 | | | KIAA0096 gene product |
| | | | | | | | | | | | | | | | is related to a protein |
| | | | | | | | | | | | | | | | kinase. |
| K126_UB | D50916_at | D50916 | PASS | 8 | 13.00 | PASS | 11 | 8 | 7.36 | 1.77 | 1.77 | UFD2 | 11 | homolog of yeast (S. | homolog of yeast (S. |
| | | | | | | | | | | | | | | cerevisiae) ufd2 | cerevisiae) ufd2 |
| E_23682 | U79288_at | U79288 | PASS | 8 | 19.13 | PASS | 12 | 8 | 10.83 | 1.77 | 1.77 | | | | |
| RPS6_rna1 | M77232_rna1 | M77232 | PASS | 9 | 274.44 | PASS | 13 | 9 | 155.46 | 1.77 | 1.77 | RP56 | 9p21 | ribosomal protein S6 | ribosomal protein S6 |
| GP250 | U60975_at | U60975 | PASS | 9 | 63.00 | PASS | 13 | 9 | 35.69 | 1.77 | 1.77 | SORL1 | 11q23.2-q24.4 | sortilin-related receptor, | sortilin-related receptor, |
| | | | | | | | | | | | | | | L(DLR class) A repeats- | L(DLR class) A repeats- |
| | | | | | | | | | | | | | | | containing |
| TTC3 | D84294_at | D84294 | PASS | 9 | 16.56 | PASS | 13 | 9 | 9.38 | 1.76 | 1.76 | | | TPRD1 | |
| FYN | M14676_at | M14676 | PASS | 9 | 31.89 | PASS | 13 | 9 | 18.08 | 1.76 | 1.76 | FYN | 6q21 | FYN oncogene related to | FYN oncogene related to |
| | | | | | | | | | | | | | | SRC, FGR, YES | SRC, FGR, YES |
| TUBFOLDC | U61234_at | U61234 | PASS | 8 | 11.38 | PASS | 11 | 86.45 | 1.76 | 1.76 | TBCC | | tubulin-specific | tubulin-speific | |
| | | | | | | | | | | | | | | chaperone c | chaperone c |
| IL10R | U00672_{'}at | U00672 | PASS | 9 | 30.22 | PASS | 13 | 9 | 17.15 | 1.76 | 1.76 | IL20RA | 11q23 | interleukin 10 receptor, | interleukin 10 receptor, |
| | | | | | | | | | | | | | | alpha | alpha |
| HG1400-HT1 | HG1400-HT1 | HG1400-HT | PASS | 9 | 11.78 | PASS | 13 | 9 | 6.69 | 1.76 | 1.76 | | | | |
| K75_{'}E2F3 | D38550_at | D38550 | PASS | 9 | 10.56 | PASS | 10 | 9 | 6.00 | 1.76 | 1.76 | E2F3 | 6p22 | E2F transcription factor 3 | E2F transcription factor 3 |
| EBP1 | U21931_at | U21931 | PASS | 9 | 23.67 | PASS | 13 | 9 | 13.46 | 1.76 | 1.76 | FBP1 | | fructose-1,6-biphosphatase | |
| DHPS | U79262_at | U79262 | PASS | 8 | 14.88 | PASS | 13 | 8 | 8.46 | 1.76 | 1.76 | | | deoxyhypusine synthase | similar to human deoxy- |
| | | | | | | | | | | | | | | | pusine synthase encoded |
| | | | | | | | | | | | | | | | by GenBank Accession |
| | | | | | | | | | | | | | | | Number L39068 |
| IGRM | X58529_at | X58529 | PASS | 9 | 112.11 | PASS | 13 | 9 | 63.85 | 1.76 | 1.76 | IGHM | 14q32.33 | | immunoglobulin mu |
| K200_MAM | D83785_at | D83785 | PASS | 8 | 12.88 | PASS | 12 | 8 | 7.33 | 1.76 | 1.76 | KIAA0200 | | | KIAA0200 gene product |
| EEF1D | Z21507_at | Z21507 | PASS | 9 | 104.11 | PASS | 13 | 9 | 59.31 | 1.76 | 1.76 | EEF1D | | eukaryotic translation | eukaryotic translation |
| | | | | | | | | | | | | | | elongation factor 1 delta | elongation factor 1 delta |
| | | | | | | | | | | | | | | (guanine nucleotide | (guanine nucleotide |
| | | | | | | | | | | | | | | exchange protein) | exchange protein) |
| HG2259-HT2 | HG2259-HT2 | HG2259-HT | PASS | 9 | 67.44 | PASS | 13 | 9 | 38.54 | 1.75 | 1.75 | | | | |
| FUCA1 | M29877_at | M29877 | PASS | 8 | 14.00 | PASS | 13 | 8 | 8.00 | 1.75 | 1.75 | FUCA1 | 1p34 | fucosidase, alpha-L-1, | fucosidase, alpha-L-1, |
| | | | | | | | | | | | | | | tissue | tissue |
| SELL | M25280_at | M25280 | PASS | 9 | 130.11 | PASS | 13 | 9 | 74.38 | 1.75 | 1.75 | LNHR | | | lymph node homing |
| | | | | | | | | | | | | | | | receptor precursor |
| M26311_s_at | M26311_s_at | M26311 | PASS | 9 | 301.44 | PASS | 13 | 9 | 172.39 | 1.75 | 1.75 | 5100A9 | 1q12-q22 | S100 calcium-binding | S100 calcium-binding |
| | | | | | | | | | | | | | | protein A9 (calgranulin B) | protein A9 (calgranulin B) |
| ANX2 | D00017_at | D00017 | PASS | 9 | 98.33 | PASS | 13 | 9 | 50.62 | 1.75 | 1.75 | ANX2 | 1.5q21-q22 | annexin II (lipocortin 11; | annexin II (lipocortin 11; |
| | | | | | | | | | | | | | | calpactin I, heavy | calpactin I, heavy |
| | | | | | | | | | | | | | | polypeptide) | polypeptide) |
| K57_COSC | D97446_at | D87446 | PASS | 9 | 9.89 | PASS | 12 | 9 | 5.67 | 1.75 | 1.75 | KIAA0257 | | | Similar to a C. elegans |
| | | | | | | | | | | | | | | | protein encoded in cosmid |
| | | | | | | | | | | | | | | | C25F2 (U40419) |
| K239_K215 | D87076_at | D97076 | PASS | 7 | 19.00 | PASS | 10 | 7 | 10.90 | 1.74 | 1.74 | KIAA0239 | | | similar to human bromo- |
| | | | | | | | | | | | | | | | domain protein BR140 |
| | | | | | | | | | | | | | | | (JC2069) |
| S79771_s_at | S78771_s_at | S78771 | PASS | 6 | 12.00 | PASS | 10 | 6 | 6.90 | 1.74 | 1.74 | | | | |
| X89109_s_at | X99109 s_at | X99109 | PASS | 9 | 107.22 | PASS | 13 | 9 | 61.69 | 1.74 | 1.74 | | | coronin homologue | |
| S92447_s_at | S82447_s_at | S92447 | PASS | 8 | 10.25 | PASS | 10 | 8 | 5.90 | 1.74 | 1.74 | GCN5L1 | 12q13-q14 | GCN5 (general control of | GCN5 (general control of |
| | | | | | | | | | | | | | | amino-acid synthesis, | amino-acid sythesis, |
| | | | | | | | | | | | | | | yeast, homolog)-like 1 | yeast, homolog-(like 1 |
| FCN1 | D83920_at | D83920 | PASS | 9 | 244.89 | PASS | 13 | 9 | 141.00 | 1.74 | 1.74 | FCN1 | 9q34 | ficolin (collagen/fibrinogen | ficolin (collagen/fibrinogen |
| | | | | | | | | | | | | | | domain-containing) 1 | domain-containing) 1 |
| HLA-DMB | U15085_at | U15085 | PASS | 9 | 54.22 | PASS | 13 | 9 | 31.23 | 1.74 | 1.74 | HLA-DMB | 6p21.3 | major histocompatability | major histocompatability |
| | | | | | | | | | | | | | | complex, class II, DM beta | complex, class II, DM beta |
| CCND2 | D13639_at | D13639 | PASS | 9 | 39.11 | PASS | 13 | 9 | 22.54 | 1.74 | 1.74 | CCND2 | 12p13 | cyclin D2 | cyclin D2 |
| CSNK1G2 | U89896_at | U89996 | PASS | 6 | 9.83 | PASS | 9 | 6 | 5.67 | 1.74 | 1.74 | | | casein kinase 1 gamma 2 | |
| TAF2H | U13991_at | U13991 | PASS | 9 | 33.44 | PASS | 13 | 9 | 19.31 | 1.73 | 1.73 | tafI130 | | TATA-binding protein | |
| | | | | | | | | | | | | | | associated factor 30 kDa | |
| | | | | | | | | | | | | | | subunit | |
| M55409_s_at | M55409_s_at | M55409 | PASS | 9 | 259.79 | PASS | 13 | 9 | 149.46 | 1.73 | 1.73 | EEF1IG | | eukaryotic translation | eukarotic translation |
| | | | | | | | | | | | | | | elongation factor 1 gamma | elongation factor 1 gamma |
| W52B2_f | HG698-HT68 | HG688-HT | PASS | 8 | 61.25 | PASS | 13 | 8 | 35.39 | 1.73 | 1.73 | | | | |
| TRNASTL | U07424_at | U07424 | PASS | 7 | 14.00 | PASS | 11 | 7 | 8.09 | 1.73 | 1.73 | FARSL | | phenylalanine-tRNA | phenylalanine-tRNA |
| | | | | | | | | | | | | | | synthetase-like | synthetase-like |
| EIF2B | M29536_at | M29536 | PASS | 9 | 24.89 | PASS | 13 | 9 | 14.38 | 1.73 | 1.73 | EIF2S2 | | eukaryotic translation | eukaryotic translation |
| | | | | | | | | | | | | | | initiation factor 2, | initiation factor 2, |
| | | | | | | | | | | | | | | subunit 2 (beta, 38 kD) | subunit 2 (beta, 38 kD) |
| E_23722 | U90909_at | U90909 | PASS | 8 | 9.75 | PASS | 11 | 8 | 5.64 | 1.73 | 1.73 | | | | |
| LGALS3 | M57710_at | M57710 | PASS | 9 | 66.89 | PASS | 13 | 9 | 39.69 | 1.73 | 1.73 | LGALS3 | 1p13 | lectin, galactosidide- | lectin, galactosidide- |
| | | | | | | | | | | | | | | binding, soluble, 3 (galactin | binding, soluble, 3 (galactin |
| | | | | | | | | | | | | | | 3) | 3) |
| PKUA | AB004884_at | AB004884 | PASS | 7 | 12.29 | PASS | 8 | 7 | 7.13 | 1.72 | 1.72 | | | PKU-alpha | PKU-alpha |
| J05016_rna1 | 305016_rna1 | 305016 | PASS | 8 | 9.88 | PASS | 11 | 8 | 5.73 | 1.72 | 1.72 | ERP70 | 7; 10 | protein disulfide isomerase | protein disulfide isomerase |
| | | | | | | | | | | | | | | related protein (calcium- | related protein (calcium- |
| | | | | | | | | | | | | | | binding protein, intestinal- | binding protein, intestinal- |
| | | | | | | | | | | | | | | | related) |
| Y09392_s_at | Y09392_s_at | Y09392 | PASS | 9 | 11.67 | PASS | 13 | 9 | 6.77 | 1.72 | 1.72 | wsl-1 | | WSL-S2 protein | |
| U23852_{'}s_at | U23852_s_at | U23852 | PASS | 9 | 66.89 | PASS | 13 | 9 | 38.85 | 1.72 | 1.72 | lck | | p56lck | truncated from of T- |
| | | | | | | | | | | | | | | | lymphocyte-specific protein |
| | | | | | | | | | | | | | | | tyrosine kinase p56lck, |
| | | | | | | | | | | | | | | | this abberant message |
| | | | | | | | | | | | | | | | encoding primarily the |
| | | | | | | | | | | | | | | | SH3 domains of p56lck |
| | | | | | | | | | | | | | | | was observed by northern |
| | | | | | | | | | | | | | | | hybridization and PCR |
| | | | | | | | | | | | | | | | amplification in poly-A |
| | | | | | | | | | | | | | | | selected RNA from two |
| | | | | | | | | | | | | | | | human leukemic T-cell |
| | | | | | | | | | | | | | | | lines. |
| P4HB | J02783_at | J02783 | PASS | 5 | 30.20 | PASS | 11 | 5 | 17.55 | 1.72 | 1.72 | P4HB | 17q25 | procollagen-proline, 2- | procollagen-proline, 2- |
| | | | | | | | | | | | | | | oxoglutarate 4-dioxy- | oxoglutarate 4-dioxy- |
| | | | | | | | | | | | | | | genase (proline 4- | genase (proline 4- |
| | | | | | | | | | | | | | | hydroxylase), beta | hydroxylase), beta |
| | | | | | | | | | | | | | | polypeptide (protein | polypeptide (protein |
| | | | | | | | | | | | | | | disulfide isomerase; thyroid | disulfide isomerase; thyroid |
| | | | | | | | | | | | | | | hormone binding protein | hormone binding protein |
| | | | | | | | | | | | | | | p55) | p55) |
| DYRK | D86550_at | D86550 | PASS | 9 | 18.67 | PASS | 13 | 9 | 10.85 | 1.72 | 1.72 | hMNB | | serine/threonine protein | human homolog of |
| | | | | | | | | | | | | | | kinase | Drosophila mnb (minibrain) |
| | | | | | | | | | | | | | | | gene |
| PSMC1 | L02426_at | L02426 | PASS | 9 | 25.67 | PASS | 13 | 9 | 14.92 | 1.72 | 1.72 | PSMC1 | 19p13.3 | proteasome (prosome, | proteasome (prosome, |
| | | | | | | | | | | | | | | macropain) 26S subunit, | macropain) 26S subunit, |
| | | | | | | | | | | | | | | ATPase, 1 | ATPase, 1 |
| CD1D | L38820_at | L38820 | PASS | 8 | 12.50 | PASS | 11 | 8 | 7.27 | 1.72 | 1.72 | CD1D | | CD1D antigen | |
| RPL9 | U09953_at | U09953 | PASS | 9 | 318.56 | PASS | 13 | 9 | 185.46 | 1.72 | 1.72 | RPL9 | 4p13 | ribosomal protein L9 | ribosomal protein L9 |
| U59877_s_at | U59877_s_at | U59877 | PASS | 6 | 15.67 | PASS | 8 | 6 | 9.13 | 1.72 | 1.72 | RAB31 | | low-Mr GTP-binding | Low Mr GTP-binding |
| | | | | | | | | | | | | | | protein Rab31 | protein of the Rab |
| | | | | | | | | | | | | | | | subfamily |
| GALT | M60091_at | M60091 | PASS | 9 | 11.44 | PASS | 9 | 9 | 6.67 | 1.72 | 1.72 | GALT | | galactose-1-phosphate | |
| | | | | | | | | | | | | | | uridyl transferase | |
| MARS | X94754_at | X94754 | PASS | 9 | 13.33 | PASS | 13 | 9 | 7.77 | 1.72 | 1.72 | MARS | 12 | yeast methionyl-tRNA | methionine-tRNA |
| | | | | | | | | | | | | | | synthetase homolog | synthetase |
| S153 | L40391_at | L40391 | PASS | 9 | 26.44 | PASS | 12 | 9 | 15.42 | 1.72 | 1.72 | | | | |
| CCNI | D50310_at | D50310 | PASS | 9 | 99.33 | PASS | 13 | 9 | 57.92 | 1.71 | 1.71 | | | cyclin I | |
| K99_PUML | D43951_at | D43951 | PASS | 9 | 12.00 | PASS | 11 | 9 | 7.00 | 1.71 | 1.71 | KIAA0099 | | | KIAA0099 gene product |
| SRI | M81637_at | M81637 | PASS | 8 | 9.00 | PASS | 8 | 8 | 5.25 | 1.71 | 1.71 | grancalcin | | grancalcin | putative |
| NDUFS8 | U65579_at | U65579 | PASS | 6 | 9.00 | PASS | 8 | 6 | 5.25 | 1.71 | 1.71 | NDUFS8 | 11q13 | NADH dehydrogenase | NADH dehydrogenase |
| | | | | | | | | | | | | | | (ubiquinone) Fe-S protein 8 | (ubiquinone) Fe-S protein 8 |
| | | | | | | | | | | | | | | (23 kD) (NADH-coenzyme | (23 kD) (NADH-coenzyme, |
| | | | | | | | | | | | | | | Q reductase) | Q reductase) |
| E_23693 | U79254_at | U79254 | PASS | 9 | 20.56 | PASS | 13 | 9 | 12.00 | 1.71 | 1.71 | | | | |
| K272_HYPC | D87462_at | D87462 | PASS | 6 | 11.50 | PASS | 7 | 6 | 6.71 | 1.71 | 1.71 | BAP1 | 3p21.31-p21.2 | BRCA1 associated protein- | BRCA1 associated protein- |
| | | | | | | | | | | | | | | 1 (ubiquitin carboxy- | 1 (ubiquitin carboxy- |
| | | | | | | | | | | | | | | terminal hydrolase) | terminal hydrolase) |
| A4 | L09604_at | L09604 | PASS | 9 | 80.22 | PASS | 13 | 9 | 46.85 | 1.71 | 1.71 | PLP2 | Xp11.23 | proteolipid protein 2 | proteolipid protein 2 |
| | | | | | | | | | | | | | | (colonic epithelium- | colonic epithelium- |
| | | | | | | | | | | | | | | enriched) | enriched) |
| SLC6A8_rna1 | U6341_rna1 | U36341 | PASS | 9 | 13.11 | PASS | 9 | 9 | 7.67 | 1.71 | 1.71 | SLC6A8 | | creatine transporter | |
| TFAP3D | U91930_at | U91930 | PASS | 9 | 20.11 | PASS | 13 | 9 | 11.77 | 1.71 | 1.71 | ADTD | 19p13.3 | adaptin, delta | adaptin, delta |
| SEPW1 | U67171_at | U67171 | PASS | 9 | 25.89 | PASS | 13 | 9 | 15 15 | 1.71 | 1.71 | SEPW1 | | selenoprotein W, 1 | selenoproein W, 1 |
| P542 | L38696_at | L38696 | PASS | 6 | 17.33 | PASS | 13 | 6 | 10.15 | 1.71 | 1.71 | | | autoantigen p542 | huRaly; hnRNP C3, N- |
| | | | | | | | | | | | | | | | terminus similar to |
| | | | | | | | | | | | | | | | huRNP C |
| ERCC5 | X69978_at | X69978 | PASS | 8 | 12.88 | PASS | 9 | 8 | 7.56 | 1.70 | 1.70 | ERCC5 | 13q22-q34 | XPG complementing | excision repair cross- |
| | | | | | | | | | | | | | | protein | complementing rodent |
| | | | | | | | | | | | | | | | repair deficiency, |
| | | | | | | | | | | | | | | | complentation group 5, |
| | | | | | | | | | | | | | | | (xeroderma pigmentosum, |
| | | | | | | | | | | | | | | | complementation group G |
| | | | | | | | | | | | | | | | (Cockayne syndrome) |
| ARF4 | M36341_at | M36341 | PASS | 9 | 17.56 | PASS | 13 | 9 | 10.31 | 1.70 | 1.70 | ARF4 | | ADP-ribosylation factor 4 | ADP-ribosylation factor 4 |
| POLRMP | U75370_at | U75370 | PASS | 7 | 10.43 | PASS | 8 | 7 | 6.13 | 1.70 | 1.70 | POLRMT | 19p13.3 | mitochondrial RNA | polymerase (RNA) |
| | | | | | | | | | | | | | | polymerase | mitochondrial (DNA |
| | | | | | | | | | | | | | | | directed |
| LUMAN | AF009368_at | AF009368 | PASS | 7 | 17.29 | PASS | 11 | 7 | 10.18 | 1.70 | 1.70 | | | Luman | basic leucine zipper |
| | | | | | | | | | | | | | | | (BZIP) protein; binds to |
| | | | | | | | | | | | | | | | herpes simplex virus VP16- |
| | | | | | | | | | | | | | | | associated host cellular |
| | | | | | | | | | | | | | | | factor (HCF); member of |
| | | | | | | | | | | | | | | | CREB/ATF protein family, |
| | | | | | | | | | | | | | | | mouse LZIP homolog |
| SAP61 | U08815_at | U08815 | PASS | 8 | 10.75 | PASS | 9 | 8 | 6.33 | 1.70 | 1.70 | SAP 61 | SAP 61 | similar to yeast PRP9, | |
| | | | | | | | | | | | | | | | Swiss-Prot Accession |
| | | | | | | | | | | | | | | | Number P19736 |
| ITK | L10717_at | L10717 | PASS | 9 | 15.67 | PASS | 13 | 9 | 9.23 | 1.70 | 1.70 | | | tyrosine kinase | 2024-2555 unique domain; |
| | | | | | | | | | | | | | | | 2556-2708 SH3 domain; |
| | | | | | | | | | | | | | | | 2750-3044 Sh2 domain |
| | | | | | | | | | | | | | | | binds phosphotyrosine- |
| | | | | | | | | | | | | | | | containing proteins); |
| | | | | | | | | | | | | | | | 3095-3884 kiase domain |
| | | | | | | | | | | | | | | | (phophorylation of tyrosine |
| | | | | | | | | | | | | | | | residues); putative |
| AFFX-HUM | AFFX-HUM | AFFX-HUM | PASS | 7 | 14.14 | PASS | 9 | 7 | 8.33 | 1.70 | 1.70 | | | | |
| EIF23 | U94855_at | U94855 | PASS | 9 | 60.00 | PASS | 13 | 9 | 35.38 | 1.70 | 1.70 | EIF3S5 | | eukaryotic translation | eukaryotic translation |
| | | | | | | | | | | | | | | initiation factor 3, | initiation factor 3, |
| | | | | | | | | | | | | | | subunit 5 (epsilon, 47 kD) | subunit 5 (epsilon, 47 kD) |
| U50079_s_at | U50079_s_at | U50079 | PASS | 7 | 22.43 | PASS | 13 | 7 | 13.23 | 1.70 | 1.70 | | | histone deacetylase HD1 | similar to S. cerevisiae |
| | | | | | | | | | | | | | | | RPD3, a global transcrip- |
| | | | | | | | | | | | | | | | tional regulator; trapoxin |
| | | | | | | | | | | | | | | | receptor |
| ISG20 | U88964_at | U88964 | PASS | 9 | 49.00 | PASS | 13 | 9 | 28.92 | 1.69 | 1.69 | ISG20 | 15q26 | interferon stimulated gene | interferon stimulated gene |
| | | | | | | | | | | | | | | (20 kD) | (20 kD) |
| CD81 | M33680_at | M33680 | PASS | 9 | 76.22 | PASS | 13 | 9 | 45.00 | 1.69 | 1.69 | CD81 | 11p15 | CD81 antigen (target of | CD81 antigen (target of |
| | | | | | | | | | | | | | | antiproliferative antibody 1) | antiproliferative antibody 1) |
| U19713_s_at | U19713_s_at | U19713 | PASS | 9 | 45.33 | PASS | 13 | 9 | 26.77 | 1.69 | 1.69 | IRT-1 | | interferon gamma | interferon gamma |
| | | | | | | | | | | | | | | responsive transcript | responsive transcript |
| APRT_rna1 | Y00486_rna1 | Y00486 | PASS | 9 | 43.22 | PASS | 13 | 9 | 25.54 | 1.69 | 1.69 | APRT | 16q24 | adenine phosphoribosyl- | adenine phosphoribosyl- |
| | | | | | | | | | | | | | | transferase | transferase |
| NF45 | U10323_at | U10323 | PASS | 9 | 24.33 | PASS | 13 | 9 | 14.38 | 1.69 | 1.69 | ILF2 | | interleuken enhancer | interleuken enhancer |
| | | | | | | | | | | | | | | binding factor 2, 45 kD | binding factor 2, 45 kD |
| IP30 | J03909_at | J03909 | PASS | 9 | 159.78 | PASS | 13 | 9 | 94.54 | 1.69 | 1.69 | | | | gamma-interferon-inducible |
| | | | | | | | | | | | | | | | protein precursor |
| ORF | M68864_at | M68864 | PASS | 9 | 36.78 | PASS | 13 | 9 | 21.77 | 1.69 | 1.69 | | | | ORF |
| RPL27A | U14968_at | U14968 | PASS | 9 | 369.33 | PASS | 13 | 9 | 218.85 | 1.69 | 1.69 | RPL27A | 11 | ribosomal protein L27a | ribosomal protein L27a |
| RENT1 | D86988_at | D86981 | PASS | 9 | 27.11 | PASS | 13 | 9 | 16.08 | 1.69 | 1.69 | RENT1 | 19 | regulator of nonsense | regulator of nonsense |
| | | | | | | | | | | | | | | transcripts 1 | transcripts 1 |
| MOV34L | D50063_at | D50063 | PASS | 9 | 19.11 | PASS | 12 | 9 | 11.33 | 1.69 | 1.69 | PSMD7 | 16q23-q2 | proteasome (prosome, | proteasome (prosome, |
| | | | | | | | | | | | | | | macropain) 26S subunit, | macropain) 26S subunit, |
| | | | | | | | | | | | | | | non-ATPase, 7 (Mov 34 | non-ATPase, 7 (Mov 34 |
| | | | | | | | | | | | | | | homolog) | homolog) |
| FHL1 | U60115_at | U60115 | PASS | 9 | 10.89 | PASS | 13 | 9 | 6.46 | 1.69 | 1.69 | FHl.1 | Xg27.2 | four and a half LIM | four and a half LIM |
| | | | | | | | | | | | | | | domains 1 | domains 1 |
| COX5B | M19961_at | M19961 | PASS | 8 | 35.88 | PASS | 13 | 8 | 21.31 | 1.68 | 1.68 | COX5B | 2cen-q13 | cytochrome c oxidase | sytochrome c oxidase |
| | | | | | | | | | | | | | | subunit Vb | subunit Vb |
| EIF4132 | U73824_at | U73824 | PASS | 9 | 48.56 | PASS | 13 | 9 | 28.85 | 1.68 | 1.68 | EIF4G2 | 11p15 | eukaryotic translation | eukaryotic translation |
| | | | | | | | | | | | | | | initiation factor 4 gamma, 2 | initiation factor 4 gamma, 2 |
| K70_KARS | D31890_at | D31890 | PASS | 9 | 31.44 | PASS | 13 | 9 | 18.69 | 1.68 | 1.68 | KIAA0070 | | | similar to lysyl tRNA |
| | | | | | | | | | | | | | | | synthetase. |
| CDC37L | U43077_at | U43077 | PASS | 9 | 24.44 | PASS | 13 | 9 | 14.54 | 1.68 | 1.68 | | | CDC37 homolog | similar to S. cerevisiae |
| | | | | | | | | | | | | | | | Cdc37p |
| SSBP | M94556_at | M94556 | PASS | 9 | 23.78 | PASS | 13 | 9 | 14.15 | 1.68 | 1.68 | SSBP | 7q34 | single-stranded DNA- | single-stranded DNA- |
| | | | | | | | | | | | | | | binding protein | binding protein |
| RGS3 | U27655_at | U27655 | PASS | 8 | 8.25 | PASS | 12 | 8 | 4.92 | 1.68 | 1.68 | | | RGP3 | |
| K190_UBP3 | D80012_at | D80012 | PASS | 8 | 14.25 | PASS | 12 | 8 | 8.50 | 1.68 | 1.68 | USP10 | | ubiquitin specific | |
| | | | | | | | | | | | | | | | protease 10 |
| FLII_rna1 | U80184_rna1 | U80184 | PASS | 7 | 18.43 | PASS | 13 | 7 | 11.00 | 1.68 | 1.68 | FLII | 17p11.2 | flightless I (Drosophilia) | flightless I (Drosophilia) |
| | | | | | | | | | | | | | | homolog | homolog |
| AIM1 | U83115_at | U83115 | PASS | 9 | 20.44 | PASS | 13 | 9 | 12.23 | 1.67 | 1.67 | AIM1 | 6q21 | non-lens beta gamma- | absent in melanoma 1 |
| | | | | | | | | | | | | | | crystallin like protein | |
| TATSF1 | U76992_at | U76992 | PASS | 7 | 10.29 | PASS | 13 | 7 | 6.15 | 1.67 | 1.67 | Tat-SF1 | | Tat-SF1 | similar to EWS and |
| | | | | | | | | | | | | | | FUS/TLS77 | |
| K235_K99 | D87078_at | D87078 | PASS | 5 | 23.40 | PASS | 13 | 5 | 14.00 | 1.67 | 1.67 | KIAA0235 | | | similar to D. melanogaster |
| | | | | | | | | | | | | | | | pumilio protein (522026); |
| | | | | | | | | | | | | | | | similar to human |
| | | | | | | | | | | | | | | | KIAA0099 protein |
| | | | | | | | | | | | | | | | (D43951) |
| HSPB1 | U12404_at | U12404 | PASS | 9 | 340.44 | PASS | 13 | 9 | 203.69 | 1.67 | 1.67 | | | Csa-19 | |
| K205_COSC | D86960_at | D86960 | PASS | 9 | 9.89 | PASS | 12 | 9 | 5.92 | 1.67 | 1.67 | KIAA0205 | | | KIAA0205 gene product |
| CTBP1 | U37408_at | U37408 | PASS | 9 | 19.67 | PASS | 13 | 9 | 11.77 | 1.67 | 1.67 | CTBP1 | 4p16 | C-terminal binding | C-terminal binding |
| | | | | | | | | | | | | | | protein 1 | protein 1 |
| EBVSRAP | HG662-HT6 | HG662-HT | PASS | 9 | 150.89 | PASS | 13 | 9 | 90.38 | 1.67 | 1.67 | | | | |
| ARHGDIB | L20688_at | L20688 | PASS | 9 | 315.00 | PASS | 13 | 9 | 188.69 | 1.67 | 1.67 | ARHGDIB | 12p12.3 | GDP dissociation inhibitor | GDP dissociation inhibitor |
| LTA4H | J03459_at | J03459 | PASS | 9 | 78.78 | PASS | 13 | 9 | 47.23 | 1.67 | 1.67 | LTA4H | 12q22 | leukotriene A4 hydrolase | leukotriene A4 hydrolase |
| ELP1 | M88458_at | M88458 | PASS | 9 | 12.44 | PASS | 13 | 9 | 7.46 | 1.67 | 1.67 | | | | |
| NP220 | D83032_at | D83032 | PASS | 9 | 10.00 | PASS | 13 | 9 | 6.00 | 1.67 | 1.67 | | | nuclear protein, NP220 | unsure initial methyonine; |
| | | | | | | | | | | | | | | | putative |
| TAP | U80073_at | U80073 | PASS | 9 | 14.44 | PASS | 12 | 9 | 8.67 | 1.67 | 1.67 | TAP | | tip associating protein | |
| EV12B | M60830_at | M60830 | PASS | 8 | 20.25 | PASS | 13 | 8 | 12.15 | 1.67 | 1.67 | | | | open reading frame |
| K249_K188 | D87436_at | D87436 | PASS | 8 | 8.88 | PASS | 12 | 8 | 5.33 | 1.66 | 1.66 | K1AA0249 | | | KIAA0249 gene product |
| CIT987SK_{'}rna | U95740_{'}rna1 | U95740 | PASS | 9 | 14.00 | PASS | 12 | 9 | 8.42 | 1.66 | 1.66 | A-362G6.1 | | Unknown gene product | |
| NASP | M97856_at | M97856 | PASS | 8 | 12.88 | PASS | 8 | 8 | 7.75 | 1.66 | 1.66 | NASP | | nuclear autoantigenic | nuclear autoantigenic |
| | | | | | | | | | | | | | | sperm protein (histone- | sperm protein (histone- |
| | | | | | | | | | | | | | | binding) | binding) |
| K94_MEAP1 | D42084_at | D42084 | PASS | 8 | 11.63 | PASS | 13 | 8 | 7.00 | 1.66 | 1.66 | KIAA0094 | | | KIAA0094 gene product |
| | | | | | | | | | | | | | | | is related to S. |
| | | | | | | | | | | | | | | | cerevisiae methionine |
| | | | | | | | | | | | | | | | aminopeptidase. |
| L10333_s_at | L10333_s_at | L10333 | PASS | 5 | 9.80 | PASS | 11 | 5 | 5.91 | 1.66 | 1.66 | NSP | | nueroendocrine-specific | |
| | | | | | | | | | | | | | | protein A | |
| H2A1L | U90551_at | U90551 | PASS | 9 | 42.22 | PASS | 13 | 9 | 25.46 | 1.66 | 1.66 | H2A/l | | histone 2A-like protein | |
| K49_IAI3B | D30756_at | D30756 | PASS | 9 | 10.44 | PASS | 13 | 9 | 6.31 | 1.66 | 1.66 | KIAA0049 | | | KIAA0049 gene product |
| DNAPK | U85611_at | U85611 | PASS | 9 | 35.89 | PASS | 13 | 9 | 21.69 | 1.65 | 1.65 | KIP | | DNA-PK interaction | |
| | | | | | | | | | | | | | | protein | |
| D13720 s_at | D13720 s_at | D13720 | PASS | 8 | 14.25 | PASS | 13 | 8 | 8.62 | 1.65 | 1.65 | | | ITK | originally named lyk |
| K241_COST | D63877_at | D63877 | PASS | 6 | 8.50 | PASS | 7 | 6 | 5.14 | 1.65 | 1.65 | KIAA0157 | | | KIAA0157 gene product is |
| | | | | | | | | | | | | | | | novel. |
| RCH1 | U28386_at | U28386 | PASS | 7 | 8.86 | PASS | 11 | 7 | 5.36 | 1.65 | 1.65 | KPNA2 | 17q23.1-q23.3 | karyopherin alpha 2 | karyopherin alpha 2 |
| | | | | | | | | | | | | | | (RAG cohort 1, importin | (RAG cohort 1, importin |
| | | | | | | | | | | | | | | alpha 1) | alpha 1) |
| K37 ADCY | D25538_at | D25538 | PASS | 9 | 17.78 | PASS | 13 | 9 | 10.77 | 1.65 | 1.65 | ADCY7 | 16q12-q13 | adenylate cyclse 7 | adenlate cyclase 7 |
| RS1 | M24194_at | M24194 | PASS | 9 | 326.89 | PASS | 13 | 9 | 198.15 | 1.65 | 1.65 | H12-3 | | MHC B complex protein | homologue, putative |
| | | | | | | | | | | | | | | 12.3 | |
| IEFSSP95O2 | L07758_at | L07758 | PASS | 9 | 9.44 | PASS | 11 | 9 | 5.73 | 1.65 | 1.65 | | | IEF SSP 9502 | nuclear phosphoprotein |
| | | | | | | | | | | | | | | | (similarity to Saccharomyes |
| | | | | | | | | | | | | | | | cerevisiae PWP1 protein) |
| K53_{'}CDC42 | D29642_at | D29642 | PASS | 9 | 27.89 | PASS | 12 | 9 | 16.92 | 1.65 | 1.65 | K1AA0053 | | | KIAA0053 gene product |
| ABR | U01147_at | U01147 | PASS | 9 | 9.89 | PASS | 12 | 9 | 6.00 | 1.65 | 1.65 | ABR | 17p13.3 | guanine nucleotide | active BCR-related |
| | | | | | | | | | | | | | | regulatory protein | gene |
| SET | M93651_at | M93651 | PASS | 9 | 24.33 | PASS | 13 | 9 | 14.77 | 1.65 | 1.65 | SET | 9q34 | SET translocation | SET translocation |
| | | | | | | | | | | | | | | (myeloid leukemia- | (myeloid leukemia- |
| | | | | | | | | | | | | | | associated) | associated) |
| K38 | D26068_at | D26068 | PASS | 9 | 5422 | PASS | 13 | 9 | 32.92 | 1.65 | 1.65 | WBSCR1 | 7q11.23 | | Williams-Beuren syndrome |
| | | | | | | | | | | | | | | | chromosome region 1 |
| PTPCAAX1 | U48296_at | U48296 | PASS | 7 | 8.43 | PASS | 8 | 7 | 5.13 | 1.64 | 1.64 | PTP4A1 | 6q12 | Protein tyrosine | protein tyrosine |
| | | | | | | | | | | | | | | phosphatase IVA1 | phosphatase type IVA, |
| | | | | | | | | | | | | | | | member 1 |
| U01691_s_at | U01691_s_at | 1301691 | PASS | 9 | 17.44 | PASS | 13 | 9 | 10.62 | 1.64 | 1.64 | ANX5 | 4q28-q32 | annexin V | annexin V (endonexin II) |
| M16591_s_at | M16591_s_at | M16591 | PASS | 9 | 29.44 | PASS | 13 | 9 | 17.92 | 1.64 | 1.64 | HCK | 20q11-q12 | hemopoietic cell kinase | hemopoietic cell kinase |
| KI02_SPC25 | D14658_at | D14658 | PASS | 9 | 22.11 | PASS | 13 | 9 | 13.46 | 1.64 | 1.64 | KIAA0102 | | | KIAA0102 gene product |
| E_23815 | U90916_at | U90916 | PASS | 8 | 11.88 | PASS | 13 | 8 | 7.23 | 1.64 | 1.64 | | | | |
| NFE2L2 | S74017_at | S74017 | PASS | 5 | 11.00 | PASS | 10 | 5 | 6.70 | 1.64 | 1.64 | Nrf2 | | Nrf2 | NF-E2-like basic leucine |
| | | | | | | | | | | | | | | | zipper transcriptional |
| | | | | | | | | | | | | | | | activator; This sequence |
| | | | | | | | | | | | | | | | comes from FIG. 1 |
| MTH1 | D16581_at | D16581 | PASS | 8 | 17.00 | PASS | 11 | 8 | 10.36 | 1.64 | 1.64 | MTH1 | 7p22 | mutT (E. coli) human | |
| | | | | | | | | | | | | | | | homolog (8-oxo-7,8- |
| | | | | | | | | | | | | | | | dihydroguanosine tri- |
| | | | | | | | | | | | | | | | phosphate |
| FKHR | U36922_at | U36922 | PASS | 9 | 17.78 | PASS | 13 | 9 | 10.85 | 1.64 | 1.64 | | | | |
| K105 | D14661_at | D14661 | PASS | 7 | 8.57 | PASS | 13 | 7 | 5.23 | 1.64 | 1.64 | KIAA0105 | 6 | gene predicted from | gene predicted from |
| | | | | | | | | | | | | | | cDNA with a complete | cDNA with a complete |
| | | | | | | | | | | | | | | coding | coding |
| CAST | D16217_at | D16217 | PASS | 9 | 20.67 | PASS | 13 | 9 | 12.62 | 1.64 | 1.64 | CAST | 5q14-q22 | calpastatin | calpastatin |
| K209_DODK | D86964_at | D86964 | PASS | 9 | 20.78 | PASS | 13 | 9 | 12.69 | 1.64 | 1.64 | DOCK2 | | | dedicator of cyto-kinesis 2 |
| X91911_s_at | X91911_s_at | X91911 | PASS | 8 | 21.13 | PASS | 13 | 8 | 12.92 | 1.63 | 1.63 | rtvp-1 | | | |
| DAPM | AFFX-DapX | AEFX-DapX | PASS | 9 | 505.56 | PASS | 13 | 9 | 309.31 | 1.63 | 1.63 | | | | |
| TCF7 | X59871_at | X59871 | PASS | 9 | 45.22 | PASS | 13 | 9 | 27.69 | 1.63 | 1.63 | TCP7 | 5q31 | transcription factor 7 | transcription factor 7 |
| | | | | | | | | | | | | | | (T-cell specific, HMG-box) | (T-cell specific, HMG-box) |
| RPS13 | HG821-HT82 | HG821-HT | PASS | 9 | 277.00 | PASS | 13 | 9 | 169.77 | 1.63 | 1.63 | | | | |
| X77588_s_at | X77588_s_at | X77588 | PASS | 8 | 10.88 | PASS | 12 | 8 | 6.67 | 1.63 | 1.63 | ARD1 | Xq28 | ARD1 N-acetyl transferase | N-acetyltransferase, |
| | | | | | | | | | | | | | | homologue | homolog of S. cerevisiae |
| | | | | | | | | | | | | | | | ARD1 |
| V00599_s_at | V00599_s_at | V00599 | PASS | 9 | 49.78 | PASS | 13 | 9 | 30.54 | 1.63 | 1.63 | | | | beta-tubulin |
| TP53 | M22898_at | M22898 | PASS | 7 | 8.00 | PASS | 11 | 7 | 4.91 | 1.63 | 1.63 | TP53 | 17p13.1 | tumor protein p53 | tumor protein p53 (Li- |
| | | | | | | | | | | | | | | | Fraumeni syndrome) |
| U31903_a_at | U31903_s_at | U31903 | PASS | 7 | 16.14 | PASS | 11 | 7 | 9.91 | 1.63 | 1.63 | CREBL1 | 6p21.3 | cAMP responsive elemtn | cAMP responsive element |
| | | | | | | | | | | | | | | binding protein-like 1 | binding protein-like 1 |
| SLC1A7 | U53347_at | U53347 | PASS | 7 | 13.57 | PASS | 12 | 7 | 8.33 | 1.63 | 1.63 | | | neutral amino acid | |
| | | | | | | | | | | | | | | transporter | |
| D13413_rna1 | D13413_rna1 | D13413 | PASS | 9 | 438.11 | PASS | 13 | 9 | 269.08 | 1.63 | 1.63 | HNRPU | | heterogeneous nuclear | heterogeneous nuclear |
| | | | | | | | | | | | | | | ribonucleoprotein U | ribonucleoprotein U |
| | | | | | | | | | | | | | | (scaffold attachment | (scaffold attachment |
| | | | | | | | | | | | | | | factor A) | factor A) |
| TGFBI | M77349_at | M77349 | PASS | 9 | 42.67 | PASS | 13 | 9 | 26.23 | 1.63 | 1.63 | TGFBI | 5q31 | transforming growth factor, | transforming growth factor, |
| | | | | | | | | | | | | | | beta-induced, 68 kD | beta-induced, 68 kD |
| K122 | D50912_at | D50912 | PASS | 6 | 8.33 | PASS | 8 | 6 | 5.13 | 1.63 | 1.63 | KIAA0122 | | | The KIAA0122 gene |
| | | | | | | | | | | | | | | | product is novel. |
| AES | U04241_at | U04241 | PASS | 9 | 65.33 | PASS | 13 | 9 | 40.23 | 1.62 | 1.62 | AES | 19p13.13 | amino-terminal enhancer of | amino-terminal enhancer of |
| | | | | | | | | | | | | | | | split |
| MAD3 | M69043_at | M69043 | PASS | 9 | 41.33 | PASS | 13 | 9 | 25.46 | 1.62 | 1.62 | NFKBIA | 14q13 | | nuclear factor of kappa |
| | | | | | | | | | | | | | | | light polypeptide gene |
| | | | | | | | | | | | | | | | enhancer in B-cells |
| | | | | | | | | | | | | | | | inhibitor, alpha |
| NME2 | HG1153-HT1 | HG1153-HT | PASS | 9 | 42.00 | PASS | 13 | 9 | 25.92 | 1.62 | 1.62 | | | | |
| K26 | D14812_at | D14812 | PASS | 9 | 36.44 | PASS | 12 | 9 | 22.50 | 1.62 | 1.62 | KIAA0026 | | | KIAA0026 gene product |
| HG4312-HT4 | HG4312-HT4 | HG4312-HT | PASS | 9 | 48.33 | PASS | 13 | 9 | 29.85 | 1.62 | 1.62 | | | | |
| TCRG | M30894_at | M30894 | PASS | 9 | 19.56 | PASS | 13 | 9 | 12.08 | 1.62 | 1.62 | CD3G | | | Ti antigen CD3-associated |
| | | | | | | | | | | | | | | | protein precursor |
| U05572_s_at | U05572_s_at | U05572 | PASS | 9 | 27.89 | PASS | 13 | 9 | 17.23 | 1.62 | 1.62 | MANB | 19cen-q13.1 | alpha-mannosidase | mannosidase, alpha B, |
| | | | | | | | | | | | | | | | lysosomal |
| M98399_s_at | M938399_s_a | M98399 | PASS | 8 | 15.38 | PASS | 12 | 8 | 9.50 | 1.62 | 1.62 | CD36 | 7q11.2 | CD36 antigen (collagen | CD36 antigen (collagen |
| | | | | | | | | | | | | | | type I receptor, thrombo- | type I receiptor, thrombo- |
| | | | | | | | | | | | | | | spondin receptor) | spondin receptor) |
| BLVRB | D26308_at | D26308 | PASS | 9 | 36.00 | PASS | 12 | 9 | 22.25 | 1.62 | 1.62 | BLVRB | 19q13.1-q13.2 | biliverdin reductase B | biliverdin reductase B |
| | | | | | | | | | | | | | | (flavin reductase | (flavin reductase |
| | | | | | | | | | | | | | | (NADPH)) | (NADPH)) |
| K147_ADCY | D63481_at | D63481 | PASS | 7 | 9.57 | PASS | 12 | 7 | 5.92 | 1.62 | 1.62 | KIAA0147 | | | The KIAA0147 gene |
| | | | | | | | | | | | | | | | product is related to |
| | | | | | | | | | | | | | | | adenylyl cyclase. |
| K115_OLIGT | D29643_at | D29643 | PASS | 9 | 26.22 | PASS | 13 | 9 | 16.23 | 1.62 | 1.62 | KIAA0115 | | | similar to Canis |
| | | | | | | | | | | | | | | | oligosaccharyltransferase |
| | | | | | | | | | | | | | | | 48 kDa subunit (M98392). |
| RPL11 | X79234_at | X79234 | PASS | 9 | 299.56 | PASS | 13 | 9 | 185.62 | 1.61 | 1.61 | RPL11 | 1 | ribosomal protein L11 | ribosomal protein L11 |
| HNRPA2B1 | M29064_at | M29064 | PASS | 9 | 34.00 | PASS | 13 | 9 | 21.08 | 1.61 | 1.61 | HNRPA2B1 | 7p15 | heterogeneous nuclear | heterogeneous nuclear |
| | | | | | | | | | | | | | | ribonucleoprotein A2/B1 | ribonucleoprotein A2/B1 |
| PSM27 | AB003177_at | AB003177 | PASS | 7 | 16.71 | PASS | 11 | 7 | 10.36 | 1.61 | 1.61 | PSMD9 | 12q24.31- | proteasome (prosome, | proteasome (prosome, |
| | | | | | | | | | | | | | q24.32 | macropain) 26S subunit, | macropain) 26S subunit, |
| | | | | | | | | | | | | | | non-ATPase,9 | non-ATPase, 9 |
| D42040_s_at | D42040_s_at | D42040 | PASS | 8 | 20.38 | PASS | 11 | 8 | 12.64 | 1.61 | 1.61 | KIAA9001 | | | KIAA9001 gene product |
| ARA9 | U78521_at | U78521 | PASS | 9 | 54.56 | PASS | 13 | 9 | 33.85 | 1.61 | 1.61 | AIP | 11q13.3 | aryl hydrocarbon receptor- | aryl hydrocarbon receptor- |
| | | | | | | | | | | | | | | interacting protein | interacting protein |
| K245_PRMA | D87432_at | D87432 | PASS | 8 | 7.88 | PASS | 9 | 8 | 4.89 | 1.61 | 1.61 | SLCTA6 | | solute carrier family 7 | solute carrier family 7 |
| | | | | | | | | | | | | | | (cationic amino acid | (cationic amino acid |
| | | | | | | | | | | | | | | transporter, y+ system), | transporter, y+ system), |
| | | | | | | | | | | | | | | member 6 | member 6 |
| GMFB | AB001106_a | AB001106 | PASS | 7 | 10.14 | PASS | 13 | 7 | 6.31 | 1.61 | 1.61 | GMFB | | glia maturation factor, | glia maturation factor, |
| | | | | | | | | | | | | | | beta | beta |
| K136 | D50926_at | D50926 | PASS | 8 | 11.50 | PASS | 13 | 8 | 7.15 | 1.61 | 1.61 | KIAA0136 | | | The KIAA0136 gene |
| | | | | | | | | | | | | | | | product is novel. |
| BM11 | L13689_at | L13689 | PASS | 9 | 9.89 | PASS | 13 | 9 | 6.15 | 1.61 | 1.61 | BM11 | 10p13 | | murine leukemia viral |
| | | | | | | | | | | | | | | | (bmi-1) oncogene homolog |
| MVK | L77213_at | L77213 | PASS | 6 | 8.83 | PASS | 10 | 6 | 5.50 | 1.61 | 1.61 | | | phosphomevalonate kinase | |
| PNN | U77718_at | U77718 | PASS | 8 | 10.50 | PASS | 13 | 8 | 6.54 | 1.61 | 1.61 | PNN | | pinin, desmosome | pinin, desmosome |
| | | | | | | | | | | | | | | associated protein | |
| 13KDDAP | U34343_at | U34343 | PASS | 9 | 27.67 | PASS | 13 | 9 | 17.23 | 1.61 | 1.61 | | | 13 kD differentiation- | |
| | | | | | | | | | | | | | | associated protein | |
| AF000424_s | AF000424_s | AF000424 | PASS | 8 | 68.25 | PASS | 13 | 8 | 42.54 | 1.60 | 1.60 | LST1 | | | cLST1/C splice variant |
| U72936_s_at | U72936_s_at | U72936 | PASS | 8 | 9.38 | PASS | 13 | 8 | 5.85 | 1.60 | 1.60 | ATRX | | putative DNA dependent | XH2; XNP; alternatively |
| | | | | | | | | | | | | | | ATPase and helicase | spliced product 1, contains |
| | | | | | | | | | | | | | | | all exons; translation starts |
| | | | | | | | | | | | | | | | in exon 9; ATRX gene |
| | | | | | | | | | | | | | | | deposited in GenBank |
| | | | | | | | | | | | | | | | Accession Numbers |
| | | | | | | | | | | | | | | | U72900-U72935 |
| M36429_s_at | M36429_s_at | M36429 | PASS | 7 | 15.29 | PASS | 13 | 7 | 9.54 | 1.60 | 1.60 | | | | transducin beta-2 subunit |
| EEF1B1 | X60489_at | X60489 | PASS | 9 | 134.56 | PASS | 13 | 9 | 84.00 | 1.60 | 1.60 | EEF1B2 | 2 | eukaryotic translation | eukaryotic translation |
| | | | | | | | | | | | | | | elongation factor 1 beta 2 | elongation factor 1 beta 2 |
| CAPZA | U56637_at | U56637 | PASS | 9 | 62.33 | PASS | 13 | 9 | 38.92 | 1.60 | 1.60 | | | capping protein alpha | |
| | | | | | | | | | | | | | | subunit isoform 1 | |
| RPL28 | U14969_at | U14969 | PASS | 9 | 444.22 | PASS | 13 | 9 | 277.62 | 1.60 | 1.60 | RPL28 | 19q13.4 | ribosomal protein L28 | ribosomal protein L28 |
| K208_DISH3 | D86963_at | D86963 | PASS | 6 | 9.33 | PASS | 12 | 6 | 5.83 | 1.60 | 1.60 | DVL3 | 3q27 | dishevelled 3 (homologous | dishevelled 3 (homologous |
| | | | | | | | | | | | | | | to Drosophila dsh) | to Drosophila dsh) |
| COL11A1 | J04177_at | J04177 | PASS | 6 | 7.00 | PASS | 8 | 6 | 4.38 | 1.60 | 1.60 | COL11A1 | 1p21 | collagen, type XI, alpha 1 | collagen, type XI, alpha 1 |
| PD123 | D14878_at | D14878 | PASS | 8 | 10.13 | PASS | 12 | 8 | 6.33 | 1.60 | 1.60 | D123 | | protein D123 | D123 gene product |
| ATP5G2_rna | X69908_rna1 | X69908 | PASS | 9 | 48.67 | PASS | 13 | 9 | 30.46 | 1.60 | 1.60 | ATP5G2 | 12 | | ATP synthase, H+ |
| | | | | | | | | | | | | | | | transporting mitochondrial |
| | | | | | | | | | | | | | | | F0 complex, subunit c |
| | | | | | | | | | | | | | | | (subunit 9), isoform 2 |
| PAP | X76770_at | X76770 | PASS | 7 | 10.14 | PASS | 11 | 7 | 6.36 | 1.59 | 1.59 | | | | |
| U49835_s_at | U49835_s_at | U49835 | PASS | 9 | 13.67 | PASS | 12 | 9 | 8.58 | 1.59 | 1.59 | CHI3L2 | 1p13.3 | YKL-39 precursor | chitinase 3-like 2 |
| CDC10 | S72008_at | S72008 | PASS | 9 | 32.33 | PASS | 13 | 9 | 20.31 | 1.59 | 1.59 | CDC10 | | cell division cycle 10 | cell division cycle 10 |
| | | | | | | | | | | | | | | (homologus to CDC10 of | (homologous to CDC10 of |
| | | | | | | | | | | | | | | S. cerevisiae) | S. cerevisiae) |
| IQGAP1 | L33075_at | L33075 | PASS | 9 | 42.00 | PASS | 13 | 9 | 26.38 | 1.59 | 1.59 | IQGAP1 | | ras GTPase-activating- | amino acid feature: IQ |
| | | | | | | | | | | | | | | like protein | calmodulin-binding |
| | | | | | | | | | | | | | | | domains, aa 740 . . . 865; |
| | | | | | | | | | | | | | | | amino acid feature: N- |
| | | | | | | | | | | | | | | | terminal repeats, aa |
| | | | | | | | | | | | | | | | 210 . . . 680; amino acid |
| | | | | | | | | | | | | | | | feature. putative GAP |
| | | | | | | | | | | | | | | | catalytic domain, aa |
| | | | | | | | | | | | | | | | 1000 . . . 1270 |
| IMPDH2_rna | L33842_rna1 | L33842 | PASS | 9 | 15.89 | PASS | 13 | 9 | 10.00 | 1.59 | 1.59 | IMPDH2 | 3p21.2 | IMP (inosine mono- | IMP (inosine mon- |
| | | | | | | | | | | | | | | phosphate) dehydrogenase | phosphate) dehydrogenase |
| | | | | | | | | | | | | | | 2 | 2 |
| RAND | D25274_at | D25274 | PASS | 9 | 26.33 | PASS | 13 | 9 | 16.62 | 1.58 | 1.58 | | | | |
| GPS1 | U20285_at | U20285 | PASS | 9 | 15.11 | PASS | 13 | 9 | 9.54 | 1.58 | 1.58 | GPS1 | | G protein pathway | G protein pathway |
| | | | | | | | | | | | | | | suppressor 1 | suppressor 1 |
| LYZ_f | J03801_f_at | J03801 | PASS | 9 | 176.33 | PASS | 13 | 9 | 111.31 | 1.58 | 1.58 | LYZ | 12 | lysozyme | lysozyme (renal |
| | | | | | | | | | | | | | | | amyloidosis) |
| SRD5A1 | M32313_at | M32313 | PASS | 7 | 8.14 | PASS | 7 | 7 | 5.14 | 1.58 | 1.58 | SRD5A1 | 5p15 | steroid-5-alpha- | steroid-5-alpha- |
| | | | | | | | | | | | | | | reductase, alpha poly- | reductase, alpha poly- |
| | | | | | | | | | | | | | | peptide 1 | peptide 1 (3-oxo-5 |
| | | | | | | | | | | | | | | | alpha-steroid delta 4- |
| | | | | | | | | | | | | | | | dehydrogenase alpha 1) |
| RPS24L | M81757_at | M81757 | PASS | 9 | 420.33 | PASS | 13 | 9 | 265.54 | 1.58 | 1.58 | RPS19 | 19q13.2 | ribosomal protein S19 | ribosomal protein S19 |
| ARC20 | AF006087_at | AF006087 | PASS | 8 | 19.63 | PASS | 10 | 8 | 12.40 | 1.58 | 1.58 | ARC20 | | p20-Arc | 20 kD subunit of the |
| | | | | | | | | | | | | | | | Arp2/3 protein complex |
| ARHA | L25080_at | L25080 | PASS | 9 | 143.56 | PASS | 13 | 9 | 90.77 | 1.58 | 1.58 | ARHA | 3p21.3 | GTP-binding protein | ras homolog gene family, |
| | | | | | | | | | | | | | | | member A |
| RPLP1 | M17886_at | M17886 | PASS | 9 | 381.67 | PASS | 13 | 9 | 241.38 | 1.58 | 1.58 | RPLP1 | | ribosomal protein, large, | ribosomal protein, large, |
| | | | | | | | | | | | | | | P1 | P1 |
| PRKAR2B | M31158_at | M31158 | PASS | 8 | 17.75 | PASS | 13 | 8 | 11.23 | 1.58 | 1.58 | PRKAR2B | 7q31-qter | protein kinase, cAMP- | protein kinase, cAMP- |
| | | | | | | | | | | | | | | dependent, regulatory, | dependent, regulatory, |
| | | | | | | | | | | | | | | type II, beta | type II, beta |
| BAG1 | Z35491_at | Z35491 | PASS | 8 | 9.63 | PASS | 11 | 8 | 6.09 | 1.58 | 1.58 | BAG1 | 9p12 | BCL2-associated | BCL2-associated |
| | | | | | | | | | | | | | | athanogene | athanogene |
| RPS20 | HG1800-HT1 | HG1800-HT | PASS | 9 | 384.89 | PASS | 13 | 9 | 243.62 | 1.58 | 1.58 | | | | |
| L43575_s_at | L43575_s_at | L43575 | PASS | 8 | 10.63 | PASS | 11 | 8 | 6.73 | 1.58 | 1.58 | | | | |
| CHS1 | U67615_at | U67615 | PASS | 7 | 10.00 | PASS | 12 | 7 | 6.33 | 1.58 | 1.58 | CHS1 | 1q42.1-q42.2 | Chediak-Higashi | Chediak-Higashi |
| | | | | | | | | | | | | | | syndrome 1 | syndrome 1 |
| RAP1A | M22995_at | M22995 | PASS | 8 | 16.13 | PASS | 13 | 8 | 10.23 | 1.58 | 1.58 | RAP1A | 1p13.3 | RAP1A, member of RAS | RAP1A, member of RAS |
| | | | | | | | | | | | | | | oncogene family | oncogene family |
| L04483_s_at | L04483_s_at | L04483 | PASS | 9 | 437.33 | PASS | 13 | 9 | 277.77 | 1.57 | 1.57 | RPS21 | 20q13.3 | ribosomal protein S21 | ribosomal protein S21 |
| KSRP | U94832_at | U94832 | PASS | 9 | 8.44 | PASS | 11 | 9 | 5.36 | 1.57 | 1.57 | | | KSRP | RNA binding protein, |
| | | | | | | | | | | | | | | | KH type RNA binding |
| | | | | | | | | | | | | | | | domain; alternative |
| | | | | | | | | | | | | | | | splicing regulator; coopera- |
| | | | | | | | | | | | | | | | tive complex formation |
| PSMB6 | D29012_at | D29012 | PASS | 9 | 24.44 | PASS | 13 | 9 | 15.54 | 1.57 | 1.57 | PSMB6 | 17p13 | proteasome subunit Y | proteasome (prosome, |
| | | | | | | | | | | | | | | | macropain) subunit, beta |
| | | | | | | | | | | | | | | | type, 6 |
| RLIP76 | L42542_at | l42542 | PASS | 8 | 11.25 | PASS | 13 | 8 | 7.15 | 1.57 | 1.57 | | | RLIP76 protein | |
| LMO2_rna1 | X61118_rna1 | X61118 | PASS | 8 | 15.00 | PASS | 13 | 8 | 9.54 | 1.57 | 1.57 | TTG- | | | |
| | | | | | | | | | | | | 2a/RBTN- | | | |
| | | | | | | | | | | | | 2a | | | |
| BPGM | X04327_at | X04327 | PASS | 7 | 7.86 | PASS | 11 | 7 | 5.00 | 1.57 | 1.57 | BPGM | 7q31-q34 | 2,3-bisphosphoglycerate | 2,3-bisphosphoglycerate |
| | | | | | | | | | | | | | | mutase | mutase |
| MTHFD | J04031_at | J04031 | PASS | 7 | 7.86 | PASS | 8 | 7 | 5.00 | 1.57 | 1.57 | MTHFD | 14q24 | | 5,10-methylenetetrahydro- |
| | | | | | | | | | | | | | | | genase, 5,10-methylene- |
| | | | | | | | | | | | | | | | tetrahydrofolate cyclo- |
| | | | | | | | | | | | | | | | hydrolase, 10-formyltetra- |
| | | | | | | | | | | | | | | | hydrofolate synthetase |
| 23KDBP | X56932_at | X56932 | PASS | 9 | 454.22 | PASS | 13 | 9 | 289.23 | 1.57 | 1.57 | | | 23 kD highly basic protein | |
| RPL17 | X57959_at | X57959 | PASS | 9 | 127.33 | PASS | 13 | 9 | 81.15 | 1.57 | 1.57 | RPL7 | 8 | ribosomal protein L7 | ribosomal protein L7 |
| RPS14_cds2 | M13934_cds2 | M13934 | PASS | 9 | 368.11 | PASS | 13 | 9 | 235.08 | 1.57 | 1.57 | RPS14 | | unknown protein | ORF; putative |
| ESD | M13450_at | M13450 | PASS | 8 | 19.88 | PASS | 13 | 8 | 12.69 | 1.57 | 1.57 | ESD | 13q14.1-q14.2 | | esterase D/formyl- |
| | | | | | | | | | | | | | | | glutathione hydrolase |
| LDLR | L00352_at | L00352 | PASS | 5 | 8.00 | PASS | 9 | 5 | 5.11 | 1.57 | 1.57 | LDLR | 19p13.3 | low density lipoprotein | low density lipoprotein |
| | | | | | | | | | | | | | | receptor (familial | receptor (familial |
| | | | | | | | | | | | | | | hypercholesterolemia) | hypercholesterolemia) |
| E_23612 | U90902_at | U90902 | PASS | 9 | 11.33 | PASS | 12 | 9 | 7.25 | 1.56 | 1.56 | | | | |
| ADD1SP2 | HG651-HT42 | HG651-HT | PASS | 9 | 20.89 | PASS | 13 | 9 | 13.38 | 1.56 | 1.56 | | | | |
| SEC61B | L25085_at | L25085 | PASS | 9 | 28.44 | PASS | 13 | 9 | 18.23 | 1.56 | 1.56 | | | Sec 61-complex beta- | |
| | | | | | | | | | | | | | | subunit | |
| U2AF1 | M96982_at | M96982 | PASS | 9 | 28.56 | PASS | 13 | 9 | 18.31 | 1.56 | 1.56 | | | U2 snRNP auxiliary | |
| | | | | | | | | | | | | | | factor small subunit | |
| HLA-DRB5_{—} | M3600_F_at | M33600 | PASS | 9 | 171.78 | PASS | 13 | 9 | 110.15 | 1.56 | 1.56 | HLA-DRB5 | 6p21.3 | major histocompatibility | major histocompatibility |
| | | | | | | | | | | | | | | complex, class II, DR | complex, class II, DR |
| | | | | | | | | | | | | | | beta 5 | beta 5 |
| XPC | D21089_at | D21089 | PASS | 7 | 17.29 | PASS | 11 | 7 | 11.09 | 1.56 | 1.56 | XPC | 3p25 | xeroderma pigmentosum, | xeroderma pigmentosum, |
| | | | | | | | | | | | | | | complementation group C | complementation group C |
| ATIC | D82348_{'}at | D82348 | PASS | 9 | 15.33 | PASS | 13 | 9 | 9.85 | 1.56 | 1.56 | ATIC | | 5-aminoimidazole-4- | 5-aminoimidazole-4- |
| | | | | | | | | | | | | | | carboxamide ribonucleotide | carboxamide ribonucleotide |
| | | | | | | | | | | | | | | formyltransferase/IMP | formyltransferase/IMP |
| | | | | | | | | | | | | | | cyclohydrolase | cyclohydrolase |
| DNMTN | U50733_at | U50733 | PASS | 9 | 15.56 | PASS | 13 | 9 | 10.00 | 1.56 | 1.56 | | | dynamitin | similar to GenBank EST |
| | | | | | | | | | | | | | | | Accession Number |
| | | | | | | | | | | | | | | | T08494; p50 subunit of |
| | | | | | | | | | | | | | | | dynactin complex |
| GBP2 | M55543_at | M55543 | PASS | 9 | 12.44 | PASS | 11 | 9 | 8.00 | 1.56 | 1.56 | GBP2 | | guanylate binding protein 2, | guanylate binding protein 2, |
| | | | | | | | | | | | | | | interferon-inducible | interferon-inducible |
| RPS12 | HG613-HT61 | HG613-HT | PASS | 9 | 290.33 | PASS | 13 | 9 | 186.69 | 1.56 | 1.56 | | | | |
| DNCLI2 | U32944_at | U32944 | PASS | 9 | 42.67 | PASS | 13 | 9 | 27.46 | 1.55 | 1.55 | PIN | 14q24 | dynein, cytoplasmic, | dynein, cytoplasmic |
| | | | | | | | | | | | | | | light polypeptide | light polypeptide |
| CTCF | U25435_at | U25435 | PASS | 7 | 8.29 | PASS | 12 | 7 | 5.33 | 1.55 | 1.55 | CTCF | | CTCF | 11-Zn-finger transcription |
| | | | | | | | | | | | | | | | factor |
| GATA3 | X58072_at | X58072 | PASS | 7 | 8.29 | PASS | 12 | 7 | 5.33 | 1.55 | 1.55 | hGATA3 | | hGATA3 transcription | |
| | | | | | | | | | | | | | | factor | |
| TRIP3 | L40410_at | L40410 | PASS | 9 | 11.00 | PASS | 12 | 9 | 7.08 | 1.55 | 1.55 | TRIP3 | | thyroid receptor interactor | thyroid hormone receptor |
| | | | | | | | | | | | | | | | interactor 3 |
| FIP1 | U41654_at | U41654 | PASS | 9 | 11.22 | PASS | 13 | 9 | 7.23 | 1.55 | 1.55 | FIP-1 | | adenovirus E3-14.7K | putative GTP-binding |
| | | | | | | | | | | | | | | interacting protein 1 | protein, homolog of small |
| | | | | | | | | | | | | | | | GTPase family members |
| L25931_s_at | L25931_s_at | L25931 | PASS | 9 | 15.78 | PASS | 12 | 9 | 10.17 | 1.55 | 1.55 | LBR | 1q42.1 | lamin B receptor | lamin B receptor |
| TFE3_rna1 | X97160_rna1 | X97160 | PASS | 7 | 9.29 | PASS | 7 | 7 | 6.00 | 1.55 | 1.55 | | | TFE3 transcription factor | |
| CYP1B1 | U03688_at | U03688 | PASS | 5 | 8.40 | PASS | 7 | 5 | 5.43 | 1.55 | 1.55 | CYP1B1 | 2p21 | cytochrome P450, sub- | chytochrome P450, sub- |
| | | | | | | | | | | | | | | family I (dioxin-inducible), | family I (dioxin-inducible), |
| | | | | | | | | | | | | | | polypeptide 1 (glaucoma 3, | polypeptide 1 (glaucoma 3, |
| | | | | | | | | | | | | | | primary infantile) | primary infantile) |
| K118_RABB | D42087_at | D42087 | PASS | 9 | 9.00 | PASS | 11 | 9 | 5.82 | 1.55 | 1.55 | KIAA0118 | | | The gene product of |
| | | | | | | | | | | | | | | | HA0793 is related to |
| | | | | | | | | | | | | | | | Dictyostelium discoideum |
| | | | | | | | | | | | | | | | RabB protein. |
| LDHB_rna1 | X13794_rna1 | X13794 | PASS | 9 | 69.00 | PASS | 13 | 9 | 44.62 | 1.55 | 1.55 | ldhB | | lactate dehydrogenase B | |
| E51 | L37368_at | L37368 | PASS | 8 | 14.75 | PASS | 13 | 8 | 9.54 | 1.55 | 1.55 | | | RNA-binding protein | putative |
| U04285_s_at | U04285_s_at | U04285 | PASS | 6 | 14.50 | PASS | 13 | 6 | 9.38 | 1.55 | 1.55 | LIPA | | lysosomal acid lipase/ | |
| | | | | | | | | | | | | | | cholesteryl ester hydrolase | |
| QDPR | M16447_at | M16447 | PASS | 7 | 7.14 | PASS | 8 | 7 | 4.63 | 1.54 | 1.54 | QDPR | 4p15.3 | quinoid dihydropteridine | quinoid dihydropteridine |
| | | | | | | | | | | | | | | reductase | reductase |
| ATP5 | M37104_at | M37104 | PASS | 9 | 11.44 | PASS | 12 | 9 | 7.42 | 1.54 | 1.54 | ATP5 | 10 | ATP synthase, H+ | ATP synthase, H+ |
| | | | | | | | | | | | | | | transporting, mitochondrial | transporting, mitochondrial |
| ATP6E | D49400_at | D49400 | PASS | 9 | 33.11 | PASS | 13 | 9 | 21.46 | 1.54 | 1.54 | ATP6S14 | 12 | ATPase, vacuolar, 14 kD | ATPase, vacuolar, 14 kD |
| TIS11D | U07802_at | U07802 | PASS | 9 | 37.56 | PASS | 13 | 9 | 24.38 | 1.54 | 1.54 | | Tis11d | | |
| U70439_s_at | U70439_s_at | U70439 | PASS | 9 | 87.67 | PASS | 13 | 9 | 56.92 | 1.54 | 1.54 | | | silver-stainable protein | similar to human PHAP12a |
| | | | | | | | | | | | | | | protein SSP29 | encoded by GenBank |
| | | | | | | | | | | | | | | | Accession Number |
| | | | | | | | | | | | | | | | Y07569 and human |
| | | | | | | | | | | | | | | | PHAP12b encoded by |
| | | | | | | | | | | | | | | | GenBank Accession |
| | | | | | | | | | | | | | | | Number Y07570 |
| CIRBP | D78134_at | D78134 | PASS | 9 | 53.89 | PASS | 13 | 9 | 35.00 | 15.4 | 1.54 | CIRBP | 19p13.3 | cold inducible RNA- | cold inducible RNA- |
| | | | | | | | | | | | | | | binding protein | binding protein |
| MAD2L1 | U68018_at | U68018 | PASS | 7 | 9.00 | PASS | 13 | 7 | 5.85 | 1.54 | 1.54 | hMAD-2 | | mad protein homolog | |
| RPL30 | HG2873-HT3 | HG2873-HT | PASS | 9 | 487.44 | PASS | 13 | 9 | 317.15 | 1.54 | 1.54 | | | | |
| ATP5O | X83218_at | X83218 | PASS | 9 | 22.44 | PASS | 13 | 9 | 14.62 | 1.54 | 1.54 | ATP5O | 21q22.1-q22.2 | ATP synthase, H+ | ATP synthase, H+ |
| | | | | | | | | | | | | | | transporting, mitochondrial | transporting, mitochondrial |
| | | | | | | | | | | | | | | F1 complex, O subunit | F1 complex, O subunit |
| | | | | | | | | | | | | | | (oligomycin sensitivity | (oligomycin sensitivity |
| | | | | | | | | | | | | | | conferring protein) | conferring protein) |
| RPL26 | HG384-HT38 | HG384-HT | PASS | 9 | 170.56 | PASS | 13 | 9 | 111.08 | 1.54 | 1.54 | | | | |
| AC002477_s | AC002477_s | AC002477 | PASS | 9 | 13.22 | PASS | 13 | 9 | 8.62 | 1.53 | 1.53 | ZNF183 | | zinc-finger protein | match to X98253 (NID |
| | | | | | | | | | | | | | | | g2274981) (PID: |
| | | | | | | | | | | | | | | | g2274982) |
| UBECS | U73379_at | U73379 | PASS | 3 | 9.20 | PASS | 10 | 5 | 6.00 | 1.53 | 1.53 | | | cyclin-selective ubiquitin | UbcH10 |
| | | | | | | | | | | | | | | carrier protein | |
| DECR | U49352_at | U49352 | PASS | 6 | 10.17 | PASS | 11 | 6 | 6.64 | 1.53 | 1.53 | DECR | 8q21.3 | 2,4-dienoyl CoA reductase | 2,4-dienoyl CoA reductase |
| X55037_s_at | X55037_s_at | X55037 | PASS | 7 | 9.86 | PASS | 9 | 7 | 6.44 | 1.53 | 1.53 | GATA3 | 10p15 | GATA-binding protein 3 | GATA-binding protein 3 |
| IL8RB | L19593_at | L19593 | PASS | 5 | 7.80 | PASS | 10 | 5 | 5.10 | 1.53 | 1.53 | IL8RB | 2q35 | interleukin 8 receptor, beta | interleukin 8 receptor, beta |
| TAF2D | U18062_at | U18062 | PASS | 9 | 8.67 | PASS | 12 | 98 | 5.67 | 1.53 | 1.53 | TAFII55 | | TFIID subunit TAFII55 | |
| DBI_rna1 | M14200_rna1 | M14200 | PASS | 8 | 16.00 | PASS | 13 | 8 | 10.46 | 1.53 | 1.53 | DBI | | | diazepam binding inhibitor |
| HG4264-HT4 | HG4264-HT4 | HG4264-HT | PASS | 9 | 14.11 | PASS | 13 | 9 | 9.23 | 1.53 | 1.53 | | | | |
| ACO2 | U80040_at | U80040 | PASS | 8 | 13.75 | PASS | 13 | 8 | 9.00 | 1.53 | 1.53 | ACO2 | 22q11.2- | aconitase 2, mitochondrial | aconitase 2, mitochondrial |
| | | | | | | | | | | | | | 113.31 | | |
| HLAE | X56841_at | X56841 | PASS | 9 | 140.78 | PASS | 13 | 9 | 92.31 | 1.53 | 1.53 | HLA-E | | HLA-E | |
| NF2TS_f | HG3236-HT3 | HG3236-Ht | PASS | 9 | 44.22 | PASS | 13 | 9 | 29.00 | 1.52 | 1.52 | | | | |
| ACAT1 | HG4073-HT4 | HG4073-HT | PASS | 9 | 9.78 | PASS | 12 | 9 | 6.42 | 1.52 | 1.52 | | | | |
| S75256_s_at | S75256_s_at | S75256 | PASS | 8 | 10.25 | PASS | 11 | 8 | 6.73 | 1.52 | 1.52 | HNL | | | neutrophil lipocalin |
| MNDA | M81750_at | M81750 | PASS | 8 | 41.13 | PASS | 13 | 8 | 27.00 | 1.52 | 1.52 | MNDA | 1q22 | myeloid cell nuclear | myeloid cell nuclear |
| | | | | | | | | | | | | | | differentiation antigen | differentiation antigen |
| TCRB | M12886_at | M12886 | PASS | 9 | 227.67 | PASS | 13 | 9 | 149.69 | 1.52 | 1.52 | TCRB | 7q35 | T-cell receptor, beta | T-cell receptor, beta |
| | | | | | | | | | | | | | | cluster | cluster |
| L09209_s_at | L09209_s_at | L09209 | PASS | 9 | 45.89 | PASS | 13 | 9 | 30.23 | 1.52 | 1.52 | APLP2 | 111q23-q25 | amyloid beta (A4) | amyloid beta (A4) |
| | | | | | | | | | | | | | | precursor-like protein 2 | precursor-like protein 2 |
| FTL | M11147_at | M11147 | PASS | 9 | 409.56 | PASS | 13 | 9 | 269.92 | 1.52 | 1.52 | FTL | 19q13.3-q13.4 | ferritin, ligh polypeptide | ferritin, light polypeptide |
| ARD1 | U14575_at | U14575 | PASS | 8 | 8.25 | PASS | 9 | 8 | 5.44 | 1.52 | 1.52 | PPP1R8 | Chr. 1 | protein phosphatase 1, | protein phosphatase 1, |
| | | | | | | | | | | | | | | regulatory (inhibitor) | regulatory (inhibitor) |
| | | | | | | | | | | | | | | subunit 8 | subunit 8 |
| HLA-A_f | M94880_f_{'}at | M94880 | PASS | 9 | 275.11 | PASS | 13 | 9 | 181.69 | 1.51 | 1.51 | | | | |
| M19311_s_at | M19311_s_at | M19311 | PASS | 9 | 108.67 | PASS | 13 | 9 | 71.77 | 1.51 | 1.51 | | | | calmodulin |
| M30448_s_at | M30448_{—l s}_at | M30448 | PASS | 9 | 58.11 | PASS | 13 | 9 | 38.38 | 1.51 | 1.51 | CSNK2B | 6p21-p12 | | casein kinase 2, beta |
| | | | | | | | | | | | | | | | polypeptide |
| BACTIN3 | AFFX-HSAC | AFFX-HSA | PASS | 9 | 385.33 | PASS | 13 | 9 | 254.54 | 1.51 | 1.51 | | | | |
| M13560_s_at | M13560_s_at | M13560 | PASS | 9 | 150.33 | PASS | 13 | 9 | 99.31 | 1.51 | 1.51 | | | cell surface glycoprotein | Ia-associated gamma chain |
| RBL38 | Z26876_at | Z26876 | PASS | 9 | 308.11 | PASS | 13 | 9 | 203.62 | 1.51 | 1.51 | RPL38 | 17 | ribosomal protein L38 | ribosomal protein L38 |
| CLIC1 | U93205_at | U93205 | PASS | 9 | 94.22 | PASS | 13 | 9 | 62.31 | 1.51 | 1.51 | CLIC1 | | chloride intracellular | chloride intracellular |
| | | | | | | | | | | | | | | channel 1 | channel 1 |
| NHC | U90549_at | U90549 | PASS | 5 | 12.40 | PASS | 10 | 5 | 8.20 | 1.51 | 1.51 | NHC | | non-histone chromosomal | |
| | | | | | | | | | | | | | | protein | |
| NPAT | D83243_at | D83243 | PASS | 9 | 9.89 | PASS | 11 | 9 | 6.55 | 1.51 | 1.51 | NPAT | 11q22-q23 | nuclear protein, ataxia- | nuclear protein, ataxia- |
| | | | | | | | | | | | | | | telangiectasia locus | telangiectasia locus |
| NRNPC | M16342_{'}at | M16342 | PASS | 9 | 25.44 | PASS | 13 | 9 | 16.85 | 1.51 | 1.51 | HNRPC | | heterogeneous nuclear ribo- | heterogeneous nuclear ribo- |
| | | | | | | | | | | | | | | nucleoprotein C (C1/C2) | nucleoprotein C (C1/C2) |
| HG1322-HT5 | HG1322-HT5 | HG1322-HT | PASS | 9 | 49.22 | PASS | 13 | 9 | 32.62 | 1.51 | 1.51 | | | | |
| SNCA | U46901_at | U46901 | PASS | 9 | 8.11 | PASS | 8 | 9 | 5.38 | 1.51 | 1.51 | SNCA | 4q21.3-q22 | synuclein, alpha (non A4 | synuclein, alpha (non A4 |
| | | | | | | | | | | | | | | component of amyloid | component of amyloid |
| | | | | | | | | | | | | | | precursor) | precursor) |
| K5 | D13630_at | D13630 | PASS | 8 | 12.75 | PASS | 11 | 8 | 8.45 | 1.51 | 1.51 | KIAA0005 | | | KIAA0005 gene product |
| HDAC1 | D50405_at | D50405 | PASS | 9 | 16.22 | PASS | 13 | 9 | 10.77 | 1.51 | 1.51 | HDAC1 | 1p34.1 | RPD3 protein | histone deacetylase 1 |
| GJA4 | Y08915_at | Y08915 | PASS | 9 | 19.11 | PASS | 13 | 9 | 12.69 | 1.51 | 1.51 | IGBP1 | Xq13.1-q13.3 | immunoglobulin (CD79A) | immunoglobulin (CD79A) |
| | | | | | | | | | | | | | | binding protein 1 | binding protein 1 |
| LUCA15 | U23946_at | U23946 | PASS | 6 | 9.33 | PASS | 10 | 6 | 6.20 | 1.51 | 1.51 | LUCA15 | | | |
| VDAC2 | L08666_at | L08666 | PASS | 9 | 19.89 | PASS | 13 | 9 | 13.23 | 1.50 | 1.50 | VDAC2 | 10q22 | voltage-dependent anion | voltage-dependent anion |
| | | | | | | | | | | | | | | channel 2 | channel 2 |
| M83667_rna1 | M83667_rna1 | M83667 | PASS | 9 | 42.67 | PASS | 13 | 9 | 28.38 | 1.50 | 1.50 | NF-IL6- | | NF-IL6-beta protein | |
| | | | | | | | | | | | | beta | | | |
| CII3 | U57877_at | U57877 | PASS | 5 | 14.80 | PASS | 13 | 5 | 9.85 | 1.50 | 1.50 | SDHC | 1q21 | succinate dehydrogenase | succinate dehydrogenase |
| | | | | | | | | | | | | | | complex, subunit C, | complex, subunit C, |
| | | | | | | | | | | | | | | integral membrane | integral membrane |
| | | | | | | | | | | | | | | protein, 15 kD | protein, 15 kD |
| U89922_s_at | U89922_s_at | U89922 | PASS | 9 | 143.56 | PASS | 13 | 9 | 95.54 | 1.50 | 1.50 | LTB | 6p21.3 | lymphotoxin B | lymphotoxin beta (TNF |
| | | | | | | | | | | | | | | | superfamily, member 3) |
| LYZ_rna1_f | X14008_rna1 | X14008 | PASS | 9 | 159.22 | PASS | 13 | 9 | 106.00 | 1.50 | 1.50 | | | lysozyme | Protein sequence is in |
| | | | | | | | | | | | | | | | conflict with the |
| | | | | | | | | | | | | | | | conceptual translation |
| NDUFV2 | M22538_at | M22538 | PASS | 8 | 28.88 | PASS | 13 | 8 | 19.23 | 1.50 | 1.50 | NDUFV2 | 18p11.31- | NADH-ubiquinone | NADH dehydrogenase |
| | | | | | | | | | | | | | p11.2 | reductase | (ubiquninone) flavoprotein |
| | | | | | | | | | | | | | | | 2 (24 kD) |
| L05072_s_at | L05072_s_at | L05072 | PASS | 9 | 17.56 | PASS | 13 | 9 | 11.69 | 1.50 | 1.50 | IRF1 | 5q23-q31 | interferon regulatory factor | interferon regulatory factor |
| | | | | | | | | | | | | | | 1 | 1 |
| K247 | D87434_at | D87434 | PASS | 8 | 10.63 | PASS | 13 | 8 | 7.08 | 1.50 | 1.50 | KIAA0247 | | | KIAA0247 gene product |
| RPS10 | U14972_at | U14972 | PASS | 9 | 350.00 | PASS | 13 | 9 | 233.31 | 1.50 | 1.50 | RPS10 | 6 | ribosomal protein S10 | ribosomal protein S10 |
| NOT56L | Y09022_at | Y09022 | PASS | 8 | 12.13 | PASS | 12 | 8 | 8.08 | 1.50 | 1.50 | not | | Not56-like protein | |
| BTG1 | X61123_at | X61123 | PASS | 9 | 54.11 | PASS | 13 | 9 | 36.08 | 1.50 | 1.50 | BTG1 | 12q22 | B-cell translocation | B-cell translocation |
| | | | | | | | | | | | | | | gene 1, anti-proliferative | gene 1, anti-proliferative |
| HMOX1 | X06985_at | X06985 | PASS | 8 | 32.63 | PASS | 13 | 8 | 21.77 | 1.50 | 1.50 | HMOX1 | 22q12 | heme oxygenase | heme oxygenase |
| | | | | | | | | | | | | | | (decycling) 1 | (decycling) 1 |
| RPS29 | U14973_at | U14973 | PASS | 9 | 390.44 | PASS | 13 | 9 | 260.54 | 1.50 | 1.50 | RPS29 | 14 | ribosomal protein S29 | ribosomal protein S29 |
| PSMHSC7 | D26599_at | D26599 | PASS | 9 | 24.22 | PASS | 12 | 9 | 16.17 | 1.50 | 1.50 | PSMB2 | 1p34.2 | proteasome (prosome, | proteasome (prosome, |
| | | | | | | | | | | | | | | macropain) subunit, beta | macropain) subunit, beta |
| | | | | | | | | | | | | | | type, 2 | type, 2 |
| SPTBN1 | M96803_at | M96803 | PASS | 5 | 10.60 | PASS | 13 | 5 | 7.08 | 1.50 | 1.50 | SPTBN1 | 2p21 | spectrin, beta, non- | spectrin, beta, non- |
| | | | | | | | | | | | | | | erythrocytic 1 | erythrocytic 1 |
| K98_TCP1 | D43950_at | D43950 | PASS | 8 | 17.63 | PASS | 13 | 8 | 11.77 | 1.50 | 1.50 | KIAA0098 | | | KIAA0098 is a human |
| | | | | | | | | | | | | | | | counterpart of mouse |
| | | | | | | | | | | | | | | | chaperonin containing |
| | | | | | | | | | | | | | | | TCP-1 gene. |
| K137_COSC | D50927_at | D50927 | PASS | 8 | 10.25 | PASS | 13 | 8 | 6.85 | 1.50 | 1.50 | KIAA0137 | | | KIAA0137 gene product |
| PAK1 | U24152_at | U24152 | PASS | 9 | 13.22 | PASS | 12 | 9 | 8.83 | 1.50 | 1.50 | PAK1 | 11q13-q14 | p21-Cdc42/Rac1-activated | p21-Cdc42/Rac1-activated |
| | | | | | | | | | | | | | | kinase 1 (yeast Ste20- | kinase 1 (yeast Ste20- |
| | | | | | | | | | | | | | | related) | related) |
| U76764_s_at | U76764_s_at | U76764 | PASS | 9 | 38.67 | PASS | 13 | 9 | 25.85 | 1.50 | 1.50 | CD97 | 19p13 | CD97 antigen | CD97 antigen |
| X65965_s_at | X65965_s_at | X65965 | PASS | 9 | 19.44 | PASS | 13 | 9 | 13.00 | 1.50 | 1.50 | | | | |
| HCPA78L | S73591_at | S73591 | PASS | 9 | 350.56 | PASS | 13 | 9 | 234.46 | 1.50 | 1.50 | brain- | | VDUP1 | 1,25-dihydroxyvitamin D-3 |
| | | | | | | | | | | | | HHCPA78 | | | up-regulated. This sequence |
| | | | | | | | | | | | | HHCPA78 | | | comes from FIG. 2. Protein |
| | | | | | | | | | | | | homolog | | | sequence is in conflict with |
| | | | | | | | | | | | | | | | the conceptual translation; |
| | | | | | | | | | | | | | | | mismatch (26[K-&get; R]) |
| S79873_s_at | S79873_s_at | S79873 | PASS | 7 | 7.29 | PASS | 8 | 7 | 4.88 | 1.49 | 1.49 | LAMP2 | Xq24 | lysosomal-associated | lysosomal-associated |
| | | | | | | | | | | | | | | membrane protein 2 | membrane protein 2 |
| L03411_s_at | L03411_s_at | L03411 | PASS | 9 | 10.00 | PASS | 13 | 9 | 6.69 | 1.49 | 1.49 | RD | 1pter-p22.1 | Radin blood group | Radin blood group |
| RPS27 | HG3214-HT3 | HG3214-HT | PASS | 9 | 438.11 | PASS | 13 | 9 | 293.46 | 1.49 | 1.49 | | | | |
| COX8_rna1 | J04823_rna1_{—} | J04823 | PASS | 9 | 80.33 | PASS | 13 | 9 | 53.85 | 1.49 | 1.49 | COX8 | 11q12-q13 | cytochrome c oxidase | cytochrome c oxidase |
| | | | | | | | | | | | | | | subunit VIII | subunit VIII |
| K127 | D50917_at | D50917 | PASS | 7 | 8.29 | PASS | 9 | 7 | 5.56 | 1.49 | 1.49 | KIAA0127 | | | KIAA0127 gene product |
| M36284_s_at | M36284_s_at | M36284 | PASS | 9 | 28.33 | PASS | 13 | 9 | 19.00 | 1.49 | 1.49 | GYPC | 2q14-q21 | glycophorin C (Gerbich | glycophorin C (Gerbich |
| | | | | | | | | | | | | | | blood group) | blood group) |
| NOF1 | U39400_at | U39400 | PASS | 8 | 11.25 | PASS | 11 | 8 | 7.55 | 1.49 | 1.49 | C11orf4 | 11q13 | NOF1 | chromosome 11 open |
| | | | | | | | | | | | | | | | reading frame 4 |
| WTRP | HG3549-HT3 | HG3549-HT | PASS | 9 | 410.22 | PASS | 13 | 9 | 275.23 | 1.49 | 1.49 | | | | |
| NELRP2 | D83018_at | D83018 | PASS | 9 | 18.22 | PASS | 13 | 9 | 12.23 | 1.49 | 1.49 | | | nel-related protein 2 | NRP2 |
| PSM445 | AB003102_at | AB003102 | PASS | 7 | 13.57 | PASS | 9 | 7 | 9.11 | 1.49 | 1.49 | PSMD11 | | proteasome (prosome, | proteasome (prosome, |
| | | | | | | | | | | | | | | macropain) 26S subunit, | macropain) 26S subunit, |
| | | | | | | | | | | | | | | non-ATPase, 11 | non-ATPase, 11 |
| UDPPH | U27460_at | U27460 | PASS | 9 | 12.22 | PASS | 9 | 9 | 8.22 | 1.49 | 1.49 | | | uridine diphosphoglucose | similar to uridine di- |
| | | | | | | | | | | | | | | pyrophosphorylase | phosphoglucose pyro- |
| | | | | | | | | | | | | | | | phosphorylase in human |
| | | | | | | | | | | | | | | | liver, Swiss-Prot |
| | | | | | | | | | | | | | | | Accession Number |
| | | | | | | | | | | | | | | | Q07131; the 5′UTR and |
| | | | | | | | | | | | | | | | 3′UTR of this clone are |
| | | | | | | | | | | | | | | | completely different from |
| | | | | | | | | | | | | | | | those of the liver form |
| DFS70 | U94319_at | U94319 | PASS | 9 | 12.00 | PASS | 13 | 9 | 8.08 | 1.49 | 1.49 | P52/P75 | | transcriptional coactivator | transcriptional coactivator |
| | | | | | | | | | | | | | | p52/p75 | p52/p75 |
| HRG4 | U40998_at | U40998 | PASS | 8 | 11.75 | PASS | 12 | 8 | 7.92 | 1.48 | 1.48 | UNC119 | 17q11.2 | retinal protein | unc119 (C. elegans) |
| | | | | | | | | | | | | | | | homolog |
| P43LSB | S75463_at | S75463 | PASS | 9 | 29.11 | PASS | 13 | 9 | 19.62 | 1.48 | 1.48 | TUFM | 16p11.2 | Tu translation elongation | Tu translation elongation |
| | | | | | | | | | | | | | | factor, mitochondrial | factor, mitochondrial |
| RPL18AH | L11566_at | L11566 | PASS | 9 | 309.00 | PASS | 13 | 9 | 208.23 | 1.48 | 1.48 | RPL18 | | ribosomal protein L18 | ribosomal protein L18 |
| ATPA | D14710_at | D14710 | PASS | 9 | 90.22 | PASS | 13 | 9 | 60.85 | 1.48 | 1.48 | ATP5A1 | 18q12-q21 | ATP synthase, II+ | ATP synthase, H+ |
| | | | | | | | | | | | | | | transporting, mitochondrial | transporting, mitochondrial |
| | | | | | | | | | | | | | | F1 complex, alpha subunit, | F1 complex, alpha subunit, |
| | | | | | | | | | | | | | | isoform 1, cardiac muscle | isoform 1, cardiac muscle |
| K261_SIS | D87450_at | D87450 | PASS | 9 | 8.67 | PASS | 13 | 9 | 5.85 | 1.48 | 1.48 | KIAA0261 | | | Similar to D. melanogaster |
| | | | | | | | | | | | | | | | parallel sister chromatids |
| | | | | | | | | | | | | | | | protein |
| HBC647 | U68494_at | U68494 | PASS | 7 | 8.00 | PASS | 10 | 7 | 5.40 | 1.48 | 1.48 | | | | |
| OAZ | D78361_at | D78361 | PASS | 9 | 383.22 | PASS | 13 | 9 | 258.69 | 1.48 | 1.48 | | | | ORF1 |
| RPL32 | X03342_at | X03342 | PASS | 9 | 420.11 | PASS | 13 | 9 | 283.85 | 1.48 | 1.48 | RPL32 | 3q13.3-q21 | ribosomal protein L32 | ribosomal protein L32 |
| U19247_rna1 | U19247_rna1 | U19247 | PASS | 9 | 14.11 | PASS | 13 | 9 | 9.54 | 1.48 | 1.48 | IFNGR1 | 6q23-q24 | interferon gamma receptor | interferon gamma receptor |
| | | | | | | | | | | | | | | 1 | 1 |
| SATB1 | M97287_at | M97287 | PASS | 9 | 19.78 | PASS | 13 | 9 | 13.38 | 1.48 | 1.48 | SATB1 | 3p23 | special AT-rich sequence | special AT-rich sequence |
| | | | | | | | | | | | | | | binding protein 1 (binds | binding protein 1 (binds |
| | | | | | | | | | | | | | | 1 (binds to nuclear matrix/ | 1 (binds to nuclear matrix/ |
| | | | | | | | | | | | | | | scaffold-associating | scaffold-associating |
| | | | | | | | | | | | | | | DNA's) | DNA's) |
| CIT987SK_rn | U96629_rna2 | U96629 | PASS | 8 | 8.13 | PASS | 8 | 8 | 5.50 | 1.48 | 1.48 | 2A8.2 | | unknown protein | |
| | | | | | | | | | | | | | | CIT987SK_2A8_1 | |
| HG1471-HT3 | HG1471-HT3 | HG1471-HT | PASS | 7 | 7.71 | PASS | 9 | 7 | 5.22 | 1.48 | 1.48 | | | | |
| CD48 | M37766_at | M37766 | PASS | 9 | 121.67 | PASS | 13 | 9 | 82.38 | 1.48 | 1.48 | CD48 | 1q21.3-q22 | CD48 antigen (B-cell | CD48 antigen (B-cell |
| | | | | | | | | | | | | | | membrane protein) | membrane protein) |
| HNRPA1_rna | X12671_rna1 | X12671 | PASS | 9 | 87.11 | PASS | 13 | 9 | 59.00 | 1.48 | 1.48 | | | hnrnp a1 protein | |
| UBE2N | D83004_at | D83004 | PASS | 9 | 9.89 | PASS | 10 | 9 | 6.70 | 1.48 | 1.48 | UBE2N | | ubiquitin-conjugating | ubiquitin-conjugating |
| | | | | | | | | | | | | | | enzyme E2N (homologous | enzyme E2N (homologous |
| | | | | | | | | | | | | | | to yeast UBC13) | to yeast UBC13) |
| GTF2A2 | U14193_at | U14193 | PASS | 8 | 11.13 | PASS | 13 | 8 | 7.54 | 1.48 | 1.48 | GTF2A1 | | general transcription | general transcription |
| | | | | | | | | | | | | | | factor IIA, 1 (37 kD and | factor IIA, 1 (37 kD and |
| | | | | | | | | | | | | | | 19 kD subunits) | 19 kD subunits) |
| T1227H | D50525_at | D50525 | PASS | 9 | 9.44 | PASS | 10 | 9 | 6.40 | 1.48 | 1.48 | | | | |
| UGALT | D87989_at | D87989 | PASS | 7 | 13.14 | PASS | 11 | 7 | 8.91 | 1.48 | 1.48 | | | UGTrell | highly similar to UDP-N- |
| | | | | | | | | | | | | | | | acetylglucosamine |
| | | | | | | | | | | | | | | transporter of K. lactis | |
| SAP145 | U41371_at | U41371 | PASS | 9 | 17.78 | PASS | 11 | 9 | 12.09 | 1.47 | 1.47 | SAP 145 | | spliceosome associated | |
| | | | | | | | | | | | | | | protein | |
| CMKRL1 | U20350_at | U20350 | PASS | 9 | 63.78 | PASS | 13 | 9 | 43.38 | 1.47 | 1.47 | CX3CR1 | 3p21 | chemokine (C-X3-C) | chemokine (C-X3-C) |
| | | | | | | | | | | | | | | receptor 1 | receptor 1 |
| RPL27 | L19527_at | L19527 | PASS | 9 | 320.89 | PASS | 13 | 9 | 218.31 | 1.47 | 1.47 | RPL27 | 17 | ribosomal protein L27 | ribosomal protein L27 |
| SPTAN1 | J05243_at | J05243 | PASS | 8 | 8.00 | PASS | 9 | 8 | 5.44 | 1.47 | 1.47 | SPTAN1 | 9q33-q34 | spectrin, alpha, non- | spectrin, alpha, non- |
| | | | | | | | | | | | | | | erythrocytic 1 (alpha- | erythrocytic 1 (alpha- |
| | | | | | | | | | | | | | | fodrin) | fodrin) |
| G22P1 | M30938_at | M30938 | PASS | 9 | 19.78 | PASS | 13 | 9 | 13.46 | 1.47 | 1.47 | | | | Ku (p70/p80) subunit |
| MCP | X59405_at | X59405 | PASS | 8 | 9.38 | PASS | 13 | 8 | 6.38 | 1.47 | 1.47 | | | | |
| K264 | D87453_at | D87453 | PASS | 5 | 7.60 | PASS | 11 | 5 | 5.18 | 1.47 | 1.47 | KIAA0264 | | | |
| SRP14 | U07857_at | U07857 | PASS | 9 | 47.78 | PASS | 13 | 9 | 32.62 | 1.46 | 1.46 | SRP14 | 15q22 | signal recognition particle | signal recognition particle |
| | | | | | | | | | | | | | | 14 kD (homologous Alu | 14 kD (homologus Alu |
| | | | | | | | | | | | | | | RNA-binding protein) | RNA-binding protein) |
| K111_NUK3 | D21853_at | D21853 | PASS | 9 | 16.00 | PASS | 13 | 9 | 10.92 | 1.46 | 1.46 | KIAA0111 | | | KIAA0111 gene product |
| IF127SEP | J04164_at | J04164 | PASS | 9 | 227.56 | PASS | 13 | 9 | 155.38 | 1.46 | 1.46 | IF117 | | interferon-induced protein | interferon-induced protein |
| | | | | | | | | | | | | | | 17 | 17 |
| RPL35 | U12465_at | U12465 | PASS | 9 | 268.44 | PASS | 13 | 9 | 183.31 | 1.46 | 1.46 | | | ribosomal protein L35 | |
| CCT6 | L27706_at | L27706 | PASS | 7 | 9.57 | PASS | 13 | 7 | 6.54 | 1.46 | 1.46 | CCT6 | | chaperonin containing T- | chaperonin containing T- |
| | | | | | | | | | | | | | | complex subunit 6 | complex subunit 6 |
| FNTA | L10413_at | L10413 | PASS | 9 | 34.33 | PASS | 13 | 9 | 23.46 | 1.46 | 1.46 | | | farnesyl-protein transferase | |
| | | | | | | | | | | | | | | alpha-subunit | |
| RPS9 | U14971_at | U14971 | PASS | 9 | 277.78 | PASS | 13 | 9 | 189.85 | 1.46 | 1.46 | RPS9 | 19q13.4 | ribosomal protein S9 | ribosomal protein S9 |
| U09510_s_at | U09510_s_at | U09510 | PASS | 9 | 8.33 | PASS | 10 | 9 | 5.70 | 1.46 | 1.46 | GARS | 7p15 | glycyl-tRNA synthetase | glycyl-tRNA synthetase |
| PECAM1 | L34657_at | L34657 | PASS | 8 | 18.75 | PASS | 12 | 8 | 12.83 | 1.46 | 1.46 | PECAM1 | 17q23 | platelet/endothelial cell | platelet/endothelial cell |
| | | | | | | | | | | | | | | adhesion molecule | adhesion molecule |
| | | | | | | | | | | | | | | (CD31 | (CD31 antigen) |
| NPC1 | AF002020_at | AF002020 | PASS | 5 | 8.60 | PASS | 9 | 5 | 5.89 | 1.46 | 1.46 | NPC1 | 18 | Niemann-Pick disease, | Niemann-Pick disease, |
| | | | | | | | | | | | | | | type C1 | type C1 |
| HSP40 | D85429_at | D85429 | PASS | 9 | 21.89 | PASS | 13 | 9 | 15.00 | 1.46 | 1.46 | HSPF1 | | heat shock protein 40 | Hsp40; Similar to bacterial |
| | | | | | | | | | | | | | | | DnaJ heat shokc protein |
| PP3CB | S46622_at | S46622 | PASS | 5 | 6.40 | PASS | 10 | 5 | 4.40 | 1.45 | 1.45 | calcineurin | | calcineurin A catalytic | This sequence comes from |
| | | | | | | | | | | | | A catalytic | | subunit | FIG. 1; calmodulin- |
| | | | | | | | | | | | | subunit, | | | dependent protein |
| | | | | | | | | | | | | calmodulin- | | | protein phosphatase |
| | | | | | | | | | | | | dependent | | | catalytic subunit; CaM-PrP |
| | | | | | | | | | | | | protein | | | catalytic subunit |
| | | | | | | | | | | | | phosphatase | | | |
| | | | | | | | | | | | | catalytic | | | |
| | | | | | | | | | | | | subunit, | | | |
| | | | | | | | | | | | | CaM-PrP | | | |
| | | | | | | | | | | | | catalytic | | | |
| | | | | | | | | | | | | subunit | | | |
| ZPK | U07358_at | U07358 | PASS | 7 | 8.86 | PASS | 11 | 7 | 6.09 | 1.45 | 1.45 | ZPK | 12q13 | serine/threonine protein | zipper (leucine) protein |
| | | | | | | | | | | | | | | kinase | kinase |
| RPL12 | L06505_at | L06505 | PASS | 9 | 221.67 | PASS | 13 | 9 | 152.46 | 1.45 | 1.45 | RPL12 | | ribosomal protein L12 | ribosomal protein L12 |
| MMP1 | M63483_at | M63483 | PASS | 5 | 12.60 | PASS | 12 | 5 | 8.67 | 1.45 | 1.45 | | | | |
| U45328_s_at | U45328_s_at | U45328 | PASS | 6 | 16.33 | PASS | 12 | 6 | 11.25 | 1.45 | 1.45 | UBE21 | 16p13.3 | ubiquitin-conjugating | ubiquitin-conjugating |
| | | | | | | | | | | | | | | enzyme E21 (homologous | enzyme E21 (homologous |
| | | | | | | | | | | | | | | to yeast UBC9) | to yeast UBC9) |
| ACTN1 | M95178_at | M95178 | PASS | 7 | 12.71 | PASS | 13 | 7 | 8.77 | 1.45 | 1.45 | ACTN1 | 14q24 | actinin, alpha 1 | actinin, alpha 1 |
| SFRS4 | L14076_at | L14076 | PASS | 9 | 11.11 | PASS | 12 | 9 | 7.67 | 1.45 | 1.45 | | | pre-mRNA splicing factor | SR protein family member; |
| | | | | | | | | | | | | | | | member; SR domain: |
| | | | | | | | | | | | | | | | (bp.583 . . . 1529); RNA |
| | | | | | | | | | | | | | | | binding domains: RNP-2 |
| | | | | | | | | | | | | | | | (bp. 57 . . . 80) and RNP-1 |
| | | | | | | | | | | | | | | | (bp. 150 . . . 173) |
| D12775_s_at | D12775_s_at | D12775 | PASS | 9 | 7.11 | PASS | 11 | 9 | 4.91 | 1.45 | 1.45 | AMPD3 | 11pter-p13 | adenosine monophosphate | adenosine monophosphate |
| | | | | | | | | | | | | | | deaminase (isoform E) | deaminase (isoform E) |
| PF4V1_rna1 | M26167_rna1 | M26167 | PASS | 6 | 7.50 | PASS | 11 | 6 | 5.18 | 1.45 | 1.45 | PF4var1 | | platelet factor 4 | precursor protein |
| ANX4 | M82809_at | M82809 | PASS | 6 | 7.83 | PASS | 12 | 6 | 5.42 | 1.45 | 1.45 | ANX4 | 2p13 | annexin IV | annexin IV (placental |
| | | | | | | | | | | | | | | | anticoagulant protein II) |
| RPL37 | HG3364-HT3 | HG3364-HT | PASS | 9 | 405.56 | PASS | 13 | 9 | 280.62 | 1.45 | 1.45 | | | | |
| HLADRA | X00274_at | X00274 | PASS | 9 | 327.33 | PASS | 13 | 9 | 226.54 | 1.44 | 1.44 | | | HLA-DR alpha heavy chain | Protein sequence is in |
| | | | | | | | | | | | | | | | conflict with the |
| | | | | | | | | | | | | | | | conceptual translation. |
| CAPZA | U03851_at | U03851 | PASS | 9 | 21.56 | PASS | 13 | 9 | 14.92 | 1.44 | 1.44 | | | capping protein alpha | similar to chicken alpha |
| | | | | | | | | | | | | | | | 2 isoform |
| CMPSIAT | D87969_at | D87969 | PASS | 9 | 10.11 | PASS | 12 | 9 | 7.00 | 1.44 | 1.44 | | | CMP-sialic acid transporter | |
| ITGB7 | S80335_at | S80335 | PASS | 9 | 16.44 | PASS | 13 | 9 | 11.38 | 1.44 | 1.44 | ITGB7 | 12q13.1 | integrin, beta 7 | integrin, beta 7 |
| ATP2A2 | M23114_at | M23114 | PASS | 5 | 6.60 | PASS | 7 | 5 | 4.57 | 1.44 | 1.44 | ATP2A2 | 12q23-q24.1 | ATPase, Ca++ transporting, | ATPase, Ca++ transporting, |
| | | | | | | | | | | | | | | cardiac muscle, slow twitch | cardiac muscle, slow twitch |
| | | | | | | | | | | | | | | 2 | 2 |
| J00105_s_at | J00150_s_at | J00105 | PASS | 9 | 422.56 | PASS | 13 | 9 | 292.69 | 1.44 | 1.44 | | | beta-2 microglobulin | |
| PSMB10_cds | X71874_cds1 | X71874 | PASS | 9 | 73.89 | PASS | 13 | 9 | 51.23 | 1.44 | 1.44 | PSMB10 | 16q22.1 | proteasome (prosome, | proteasome (prosome, |
| | | | | | | | | | | | | | | macropain) subunit, beta | macropain) subunit, beta |
| | | | | | | | | | | | | | | type, 10 | type, 10 |
| CLARP | AF005775_at | AF005775 | PASS | 8 | 9.00 | PASS | 12 | 8 | 6.25 | 1.44 | 1.44 | CFLAR | 2q33-q34 | CASP8 and FADD-like | CASP8 and FADD-like |
| | | | | | | | | | | | | | | apoptosis regulator | apoptosis regulator |
| NTRK1 | X66397_{—l at} | X66397 | PASS | 9 | 15.22 | PASS | 12 | 9 | 10.58 | 1.44 | 1.44 | TPR | 1 | translocated promotor | translocated promoter |
| | | | | | | | | | | | | | | region (to activated | region (to activated |
| | | | | | | | | | | | | | | MET oncogene) | MET oncogene) |
| TRA2A | U53209_at | U53209 | PASS | 7 | 22.14 | PASS | 10 | 7 | 15.40 | 1.44 | 1.44 | htra-2 alpha | | transformer-2 alpha | pre-mRNA processing |
| | | | | | | | | | | | | | | | factor |
| ATP5B | M19483_at | M19483 | PASS | 9 | 46.22 | PASS | 13 | 9 | 32.15 | 1.44 | 1.44 | ATP5B | 12p13-pter | ATP synthase, H+ | ATP synthase, H+ |
| | | | | | | | | | | | | | | transporting, mitochondrial | transporting, mitochondrial |
| | | | | | | | | | | | | | | F1 complex, beta poly- | F1 complex, beta poly- |
| | | | | | | | | | | | | | | peptide | peptide |
| RPL30 | HG311-HT31 | HG311-HT | PASS | 9 | 202.44 | PASS | 13 | 9 | 140.85 | 1.44 | 1.44 | | | | |
| M36430_s_at | M36430_s_at | M36430 | PASS | 7 | 12.71 | PASS | 13 | 7 | 8.85 | 1.44 | 1.44 | GNB1 | 1p36.21-36.33 | | guanine nucleotide binding |
| | | | | | | | | | | | | | | | protein (G protein), beta |
| | | | | | | | | | | | | | | | polypeptide 1 |
| IGHMBP2 | V00563_at | V00563 | PASS | 9 | 42.56 | PASS | 13 | 9 | 29.62 | 1.44 | 1.44 | | | | reading frame (part 8) |
| | | | | | | | | | | | | | | | (1 is 2nd base in codon) |
| RPL17 | X55954_at | X55954 | PASS | 9 | 294.00 | PASS | 13 | 9 | 204.69 | 1.44 | 1.44 | RPL23 | 17q | ribosomal protein L23 | ribosomal protein L23 |
| RPL4 | D23660_at | D23660 | PASS | 9 | 297.78 | PASS | 13 | 9 | 207.46 | 1.44 | 1.44 | RPL4 | 15 | ribosomal protein L4 | ribosomal protein L4 |
| D63861_s_at | D63861_s_at | D63861 | PASS | 7 | 7.71 | PASS | 8 | 7 | 5.38 | 1.44 | 1.44 | hCyP40 | | cyclophilin 40 | |
| ZNF134 | U09412_at | U09412 | PASS | 7 | 6.86 | PASS | 9 | 7 | 4.78 | 1.44 | 1.44 | ZNF134 | 19q13.4 | zinc finger protein 134 | zinc finger protein 134 |
| | | | | | | | | | | | | | | (clone pHZ-15) | (clone pHZ-15) |
| ATP1B3 | U51478_at | U51478 | PASS | 9 | 32.11 | PASS | 13 | 9 | 22.38 | 1.43 | 1.43 | ATP1B3 | 3q22-q23 | ATPase, Na+/K+ | ATPase, Na+/K+ |
| | | | | | | | | | | | | | | transporting, beta 3 | transporting, beta 3 |
| | | | | | | | | | | | | | | polypeptide | polypeptide |
| STX4A | U07158_at | U07158 | PASS | 9 | 11.22 | PASS | 12 | 9 | 7.83 | 1.43 | 1.43 | STX4A | | syntaxin 4A (placental) | syntaxin 4A (placental) |
| HO3 | U18937_at | U18937 | PASS | 8 | 7.88 | PASS | 10 | 8 | 5.50 | 1.43 | 1.43 | HO3 | | histidyl-tRNA synthetase | |
| | | | | | | | | | | | | | | homologue | |
| CD3Z | J04132_at | J04132 | PASS | 9 | 31.56 | PASS | 13 | 9 | 22.08 | 1.43 | 1.43 | CD3Z | 1q22-q25 | CD3Z antigen, zeta poly- | CD3Z antigen, zeta poly- |
| | | | | | | | | | | | | | | peptide (TiT3 complex) | peptide (TiT3 complex) |
| K41_ZNFGC | D26069_at | D26069 | PASS | 7 | 8.43 | PASS | 10 | 7 | 5.90 | 1.43 | 1.43 | KIAA0041 | | | |
| CD79B | M89957_at | M89957 | PASS | 6 | 12.33 | PASS | 11 | 6 | 8.64 | 1.43 | 1.43 | CD79B | 17q23 | cell surface glycoprotein | CD79B antigen (immuno- |
| | | | | | | | | | | | | | | | globulin-associated |
| | | | | | | | | | | | | | | | beta) |
| UQCRFS1 | L32977_at | L32977 | PASS | 9 | 27.22 | PASS | 13 | 9 | 19.08 | 1.43 | 1.43 | UQCRFS1 | | Rieske Fe-S protein | |
| PHC | X60036_at | X60036 | PASS | 9 | 57.89 | PASS | 13 | 9 | 40.62 | 1.43 | 1.43 | PHC | 12 | phosphate carrier, | phosphate carrier, |
| | | | | | | | | | | | | | | mitochondrial | mitochondrial |
| RPS16 | M60854_at | M60854 | PASS | 9 | 411.67 | PASS | 13 | 9 | 289.15 | 1.42 | 1.42 | RPS16 | 19q | ribosomal protein S16 | ribosomal protein S16 |
| TMSB4 | M17733_at | M17733 | PASS | 9 | 430.44 | PASS | 13 | 9 | 302.69 | 1.42 | 1.42 | TMSB4Y | Y | thymosin, beta 4, Y | thymosin, beta 4, Y |
| | | | | | | | | | | | | | | chromosome | chromosome |
| HMGCL | L07033_at | L07033 | PASS | 8 | 7.75 | PASS | 11 | 8 | 5.45 | 1.42 | 1.42 | HMGCL | 1 | 3-hydroxymethyl-3- | 3-hydroxymethyl-3- |
| | | | | | | | | | | | | | | methylglutaryl-Coenzyme | methylglutaryl-Coenzyme |
| | | | | | | | | | | | | | | A lyase (hydroxy- | A lyase (hydroxy- |
| | | | | | | | | | | | | | | methylglutaricaciduria) | methylglutaricaciduria) |
| RPSKA2 | U08316_at | U08316 | PASS | 3 | 8.00 | PASS | 11 | 5 | 5.64 | 1.42 | 1.42 | RPS6KA3 | Xp22.2-p22.1 | ribosomal protein S6 | ribosomal protein S6 |
| | | | | | | | | | | | | | | kinase, 90 kD, polypeptide | kinase, 90 kD, polypeptide |
| | | | | | | | | | | | | | | 3 | 3 |
| M96995_s_at | M96995_s_at | M96995 | PASS | 6 | 18.00 | PASS | 13 | 6 | 12.69 | 1.42 | 1.42 | GRB2 | 17q24-q25 | growth factor receptor- | growth factor receptor- |
| | | | | | | | | | | | | | | bound protein 2 | bound protein 2 |
| U43901_rna1 | U43901_rna1 | U43901 | PASS | 9 | 287.89 | PASS | 13 | 9 | 203.08 | 1.42 | 1.42 | | | 37 kD laminin receptor | 37LRP/p40, metastasis- |
| | | | | | | | | | | | | | | precursor/p40 ribosome | associated multifunctional |
| | | | | | | | | | | | | | | associated protein | protein |
| RPL37A | L06499_at | L06499 | PASS | 9 | 428.44 | PASS | 13 | 9 | 302.38 | 1.42 | 1.42 | RPL37A | | ribosomal protein L37a | ribosomal protein L37a |
| GLB1 | M83822_at | M83822 | PASS | 7 | 7.43 | PASS | 12 | 7 | 5.25 | 1.41 | 1.41 | BGL | | beige-like protein | similar to yeast YCR032w, |
| | | | | | | | | | | | | | | | GenBank Accession |
| | | | | | | | | | | | | | | | Number X59720, Mus |
| | | | | | | | | | | | | | | | musculus BG, GenBank |
| | | | | | | | | | | | | | | | Accession Number |
| | | | | | | | | | | | | | | | U52461 and C. elegans |
| | | | | | | | | | | | | | | | F10F2.1, GenBank |
| | | | | | | | | | | | | | | | Accession Number |
| | | | | | | | | | | | | | | | Z35598; previously |
| | | | | | | | | | | | | | | | identified as CDC4L |
| F13A1 | M14539_at | M14539 | PASS | 9 | 78.00 | PASS | 13 | 9 | 55.15 | 1.41 | 1.41 | F13A1 | 6p24.2-p23 | coagulation factor XIII, | coagulation factor XIII, |
| | | | | | | | | | | | | | | A1 polypeptide | A1 polypeptide |
| M26708_s_at | M26708_s_at | M26708 | PASS | 9 | 183.67 | PASS | 13 | 9 | 130.00 | 1.41 | 1.41 | PTMA | | | prothymosin alpha |
| K02777_s_at | K02777_s_at | K02777 | PASS | 7 | 25.86 | PASS | 13 | 7 | 18.31 | 1.41 | 1.41 | TCRA | 14q11.2 | T-cell receptor, alpha | T-cell receptor, alpha |
| | | | | | | | | | | | | | | (V, D, J, C) | (V, D, J, C) |
| ITRAF | U59863_at | U59863 | PASS | 6 | 6.67 | PASS | 11 | 6 | 4.73 | 1.41 | 1.41 | | | I-TRAF | |
| RPS24L | X62691_at | X62691 | PASS | 9 | 325.00 | PASS | 13 | 9 | 230.46 | 1.41 | 1.41 | RPS15A | 16p | ribosomal protein S15a | ribosomal protein S15a |
| MIHB | U37547_at | U37547 | PASS | 7 | 9.71 | PASS | 9 | 7 | 6.89 | 1.41 | 1.41 | AP12 | 11q22 | apoptosis inhibitor 2 | apoptosis inhibitor 2 |
| TRA2B | U68063_at | U68063 | PASS | 8 | 13.13 | PASS | 13 | 8 | 9.31 | 1.41 | 1.41 | SFRS10 | 3q | splicing factor, arginine/ | splicing factor, arginine/ |
| | | | | | | | | | | | | | | serine-rich (transformer | serine-rich (transformer |
| | | | | | | | | | | | | | | 2 Drosophila homolog) 10 | 2 Drosophila homolog) 10 |
| POLR2F | Z27113_at | Z27113 | PASS | 9 | 22.33 | PASS | 13 | 9 | 15.85 | 1.41 | 1.41 | | | RNA Polymerase II | |
| | | | | | | | | | | | | | | subunit 14 4 kD | |
| RPL8 | Z28407_at | Z28407 | PASS | 9 | 324.56 | PASS | 13 | 9 | 230.46 | 1.41 | 1.41 | RPL8 | 8 | ribosomal protein L8 | ribosomal protein L8 |
| K158_DIFF6 | D63878_at | D63878 | PASS | 8 | 34.13 | PASS | 12 | 8 | 24.25 | 1.41 | 1.41 | DIFF6 | 2q37 | differentiation 6 | differentiation 6 |
| | | | | | | | | | | | | | | (deoxyguanosine | (deoxyguanosine |
| | | | | | | | | | | | | | | triphosphate tri- | triphosphate tri- |
| | | | | | | | | | | | | | | phosphohydrolase) | phosphohydrolase) |
| M97935_s_at | M97935_s_at | M97935 | PASS | 9 | 20.44 | PASS | 13 | 9 | 14.54 | 1.41 | 1.41 | | | transcription factor ISGF-3 | |
| S171 | L40393_at | L40393 | PASS | 8 | 8.25 | PASS | 8 | 8 | 5.88 | 1.40 | 1.40 | NUMB | 14q24.3 | numb (Drosophila) | numb (Drosophila) |
| | | | | | | | | | | | | | | homolog | homolog |
| HG3638-HT3 | HG3638-HT3 | HG3638-HT | PASS | 5 | 9.20 | PASS | 9 | 5 | 6.56 | 1.40 | 1.40 | | | | |
| K171_HYPL | D79993_at | D79993 | PASS | 6 | 7.83 | PASS | 12 | 6 | 5.58 | 1.40 | 1.40 | KIAA0171 | | | KIAA0171 gene product |
| YSEC7 | M85169_at | M85169 | PASS | 9 | 22.56 | PASS | 13 | 9 | 16.08 | 1.40 | 1.40 | PSCD1 | 17q25 | pleckstrin homology, | pleckstrin homology, |
| | | | | | | | | | | | | | | Sec7 and coiled/coil | Sec 7 and coiled/coil |
| | | | | | | | | | | | | | | domains 1 (cytohesin 1) | domains 1 (cytohesin 1) |
| PRCP | L13977_at | L13977 | PASS | 9 | 14.67 | PASS | 11 | 9 | 10.45 | 1.40 | 1.40 | PRCP | 11q14 | prolylcaqrboxypeptidase | prolylcarboxypeptidase |
| | | | | | | | | | | | | | | | (angiotensinase C) |
| M4P | L03532_at | L03532 | PASS | 9 | 25.89 | PASS | 13 | 9 | 18.46 | 1.40 | 1.40 | | | M4 protein | |
| ARC41 | AF006084_at | AF006084 | PASS | 9 | 88.22 | PASS | 13 | 9 | 62.92 | 1.40 | 1.40 | ARC41 | p41-Arc | WE repeat containing | |
| | | | | | | | | | | | | | | | protein; similar to Sop2Hs; |
| | | | | | | | | | | | | | | | 41 kD subunit of the |
| | | | | | | | | | | | | | | | Arp2/3 protein complex |
| K159_CHR1 | D63880_at | D63880 | PASS | 8 | 7.00 | PASS | 9 | 8 | 5.00 | 1.40 | 1.40 | KIAA0159 | | | KIAA0159 gene product |
| E_POV2 | U18919_at | U18919 | PASS | 5 | 6.80 | PASS | 7 | 5 | 4.86 | 1.40 | 1.40 | | | | |
| AQP3 | AB001325_at | AB001325 | PASS | 9 | 20.56 | PASS | 13 | 9 | 14.69 | 1.40 | 1.40 | AQP3 | 9p13 | aquaporin 3 | aquaporin 3 |
| ATP5G3 | U09813_at | U09813 | PASS | 9 | 33.44 | PASS | 13 | 9 | 23.92 | 1.40 | 1.40 | P3 | | mitochondrial ATP | |
| | | | | | | | | | | | | | | synthase subunit 9 | |
| | | | | | | | | | | | | | | precursor | |
| STAT3 | L29277_at | L29277 | PASS | 8 | 10.13 | PASS | 12 | 8 | 7.25 | 1.40 | 1.40 | STAT3 | 17q21 | signal transducer and | signal transducer and |
| | | | | | | | | | | | | | | activator of transcription | activator of transcription |
| | | | | | | | | | | | | | | 3 (acute-phase response | 3 (acute-phase response |
| | | | | | | | | | | | | | | factor | factor |
| CSE1 | U33286_at | U33286 | PASS | 8 | 8.75 | PASS | 11 | 8 | 6.27 | 1.39 | 1.39 | CSE1L | 20q13 | chromosome segregation 1 | chromosome segregation 1 |
| | | | | | | | | | | | | | | (yeast homolog)-like | (yeast homolog)-like |
| HNRPG | Z23064_at | Z23064 | PASS | 9 | 12.33 | PASS | 13 | 9 | 8.85 | 1.39 | 1.39 | HNRPG | 6p12 | heterogeneous nuclear | heterogeneous nuclear |
| | | | | | | | | | | | | | | ribonucleoprotein G | ribonucleoprotein G |
| U61397_s_at | U61397_s_at | U61397 | PASS | 9 | 9.22 | PASS | 13 | 9 | 6.62 | 1.39 | 1.39 | UBL1 | 2q32.2-q33 | ubiquitin-like 1 (sentrin) | ubiquitin-like 1 (sentrin) |
| CD53 | M37033_at | M37033 | PASS | 9 | 103.22 | PASS | 13 | 9 | 74.08 | 1.39 | 1.39 | CD53 | 1p31-p12 | CD53 glycoprotein | CD53 antigen |
| RPL7A | M36072_at | M36072 | PASS | 9 | 272.56 | PASS | 13 | 9 | 195.62 | 1.39 | 1.39 | RPL7A | 9q33-q34 | ribosomal protein L7a | ribosomal protein L7a |
| ATP7A | AB000409_a | AB000409 | PASS | 9 | 9.89 | PASS | 10 | 9 | 7.10 | 1.39 | 1.39 | MKNK1 | | MAP kinase-interacting | MAP kinase-interacting |
| | | | | | | | | | | | | | | serine/threonine kinase 1 | serine/threonine kinase 1 |
| RPS8_rna1 | X67247_rna1 | X67247 | PASS | 9 | 359.78 | PASS | 13 | 9 | 258.46 | 1.39 | 1.39 | RPS8 | qp34.1-p32 | ribosomal protein S8 | ribosomal protein S8 |
| K184 | D80006_at | D80006 | PASS | 9 | 20.44 | PASS | 13 | 9 | 14.69 | 1.39 | 1.39 | KIAA0184 | | | |
| RPL6 | X69391_at | X69391 | PASS | 9 | 178.33 | PASS | 13 | 9 | 128.23 | 1.39 | 1.39 | RPL6 | 12q23-q24.1 | ribosomal protein L6 | ribosomal protein L6 |
| SHGC | M88108_at | M88108 | PASS | 9 | 22.44 | PASS | 13 | 9 | 16.15 | 1.39 | 1.39 | p62 | | p62 | |
| K107 | D14663_at | D14663 | PASS | 7 | 11.86 | PASS | 13 | 7 | 8.54 | 1.39 | 1.39 | KIAA0107 | | | KIAA0107 gene product |
| EIF3 | U46025_at | U46025 | PASS | 9 | 38.33 | PASS | 13 | 9 | 27.62 | 1.39 | 1.39 | EIF3S8 | 16p11.2 | eukaryotic translation | eukaryotic translation |
| | | | | | | | | | | | | | | initiation factor 3, | initiation factor 3, |
| | | | | | | | | | | | | | | subunit 8 (110 kD) | subunit 8 (110 kD) |
| K64 | D31764_at | D31764 | PASS | 5 | 14.20 | PASS | 13 | 5 | 10.23 | 1.39 | 1.39 | KIAA0064 | | | KIAA0064 gene product |
| PTPN12 | M93425_at | M93425 | PASS | 9 | 16.22 | PASS | 13 | 9 | 11.69 | 1.39 | 1.39 | PTPN12 | 7q11.23 | protein tyrosine | protein tyrosine |
| | | | | | | | | | | | | | | phosphatase, non-receptor | phosphatase, non-receptor |
| | | | | | | | | | | | | | | type 12 | type 12 |
| ACERA | U70063_at | U70063 | PASS | 7 | 13.71 | PASS | 9 | 7 | 9.89 | 1.39 | 1.39 | ASAH | | N-acylsphingosine amido- | N-acylsphingosine amido- |
| | | | | | | | | | | | | | | hydrolase | hydrolase |
| RPL5 | HG4319-HT4 | HG4319-HT | PASS | 9 | 351.78 | PASS | 13 | 9 | 253.69 | 1.39 | 1.39 | | | | |
| ATPBPCD | D64158_at | D64158 | PASS | 7 | 8.57 | PASS | 11 | 7 | 6.18 | 1.39 | 1.39 | | | ATP binding protein | APACD:ATP binding |
| | | | | | | | | | | | | | | | protein associated with |
| | | | | | | | | | | | | | | | cell differentiation |
| S100A10 | M38591_at | M38591 | PASS | 9 | 75.56 | PASS | 13 | 9 | 54.54 | 1.39 | 1.39 | S100A10 | 1q21 | S100 calcium-binding | S100 calcium-binding |
| | | | | | | | | | | | | | | protein A10 (annexin II | protein A10 (annexin II |
| | | | | | | | | | | | | | | ligand, capactin I, light | ligand, capactin I, light |
| | | | | | | | | | | | | | | polypeptide | polypeptide (p11)) |
| GP25L2 | X90872_at | X90872 | PASS | 8 | 15.13 | PASS | 13 | 8 | 10.92 | 1.38 | 1.38 | gp2512 | | | associated to Golgi |
| | | | | | | | | | | | | | | | apparatus |
| K225_COSK | D86978_at | D86978 | PASS | 8 | 8.00 | PASS | 9 | 8 | 5.78 | 1.38 | 1.38 | KIAA0225 | | | similar to a C. elegans |
| | | | | | | | | | | | | | | | protein encoded in cosmid |
| | | | | | | | | | | | | | | | K12D12(Z49069) |
| SLC20A1 | L20859_at | L20859 | PASS | 9 | 7.89 | PASS | 10 | 9 | 5.70 | 1.38 | 1.38 | GLVR1 | | leukemia virus receptor 1 | |
| U79528_s_at | U79528_s_at | U79528 | PASS | 9 | 15.00 | PASS | 13 | 9 | 10.85 | 1.38 | 1.38 | | | SR31747 binding protein 1 | sterol isomerase 1; SR-BP1 |
| RPS5 | U14970_at | U14970 | PASS | 9 | 293.11 | PASS | 13 | 9 | 212.00 | 1.38 | 1.38 | RPS5 | 19q13.4 | ribosomal protein S5 | ribosomal protein S5 |
| AFFX-HSAC | AFFX-HSAC | AFFX-HSA | PASS | 7 | 19.14 | PASS | 13 | 7 | 13.85 | 1.38 | 1.38 | | | | |
| MCM7 | D55716_at | D55716 | PASS | 6 | 9.50 | PASS | 8 | 6 | 6.88 | 1.38 | 1.38 | Plcdc47 | | Plcdc47 | |
| PBP1 | U83463_at | U83463 | PASS | 8 | 10.63 | PASS | 13 | 8 | 7.69 | 1.38 | 1.38 | | | scaffold protein Pbp1 | |
| MDCR | U72342_at | U72342 | PASS | 9 | 12.11 | PASS | 13 | 9 | 8.77 | 1.38 | 1.38 | PAFAH1B1 | 17p13.3-p13.3 | platelet-activating factor | platelet-activating factor |
| | | | | | | | | | | | | | | acetylhydrolase, isoform | acetylhydrolase, isoform |
| | | | | | | | | | | | | | | 1b, alpha subunit (45 kD) | 1b, alpha subunit (45 kD) |
| RPI | L35035_at | L35035 | PASS | 8 | 7.25 | PASS | 8 | 8 | 5.25 | 1.38 | 1.38 | | | | |
| UBE2D2 | U39317_at | U39317 | PASS | 9 | 14.22 | PASS | 13 | 9 | 10.31 | 1.38 | 1.38 | UBE2D3 | | ubiquitin-conjugating | ubiquitin-conjugating |
| | | | | | | | | | | | | | | enzyme E2D 3 (homolo- | enzyme E2D 3 (homolo- |
| | | | | | | | | | | | | | | gous to yeast UBC4/5) | gous to yeast UBC4/5) |
| DDX | U90426_at | U90426 | PASS | 9 | 11.67 | PASS | 13 | 9 | 8.46 | 1.38 | 1.38 | | | nuclear RNA helicase | DEAD-box family member; |
| | | | | | | | | | | | | | | | contains DECD-box, |
| | | | | | | | | | | | | | | | similar to rat liver |
| | | | | | | | | | | | | | | | nuclear protein p47 (PIR |
| | | | | | | | | | | | | | | | Accession Number |
| | | | | | | | | | | | | | | | A42881) and D. |
| | | | | | | | | | | | | | | | melanogaster DEAD-box |
| | | | | | | | | | | | | | | | RNA helicase WM6 (PIR |
| | | | | | | | | | | | | | | | Accession Number S51601) |
| RPS25 | M64716_at | M64716 | PASS | 9 | 275.67 | PASS | 13 | 9 | 200.15 | 1.38 | 1.38 | PRS25 | 11q23.3 | ribosomal protein S25 | ribosomal protein S25 |
| PSMB8_rna1 | Z14982_rna1 | Z14982 | PASS | 8 | 30.75 | PASS | 12 | 8 | 22.33 | 1.38 | 1.38 | MHC- | | proteasome subunit LMP7 | alternative splicing |
| | | | | | | | | | | | | encoded | | | |
| | | | | | | | | | | | | proteasome | | | |
| | | | | | | | | | | | | subunit gene | | | |
| HG3730-HT4 | HG3730-HT4 | HG3730-HT | PASS | 8 | 9.75 | PASS | 12 | 8 | 7.08 | 1.38 | 1.38 | | | | |
| RPL21 | U25789_at | U25789 | PASS | 9 | 153.78 | PASS | 13 | 9 | 111.77 | 1.38 | 1.38 | RPL21 | 13 | ribosomal protein L21 | ribosomal protein L21 |
| DUT | U31930_at | U31930 | PASS | 9 | 11.11 | PASS | 13 | 9 | 8.08 | 1.38 | 1.38 | DUT | 15q15-q21.1 | dUTP pyrophosphatase | duTP pyrophosphatase |
| EIF4G2 | D86549_at | D86549 | PASS | 8 | 6.88 | PASS | 1 | 8 | 5.00 | 1.38 | 1.38 | | | p97 homologous protein | |
| K232 | D86985_at | D86985 | PASS | 6 | 7.00 | PASS | 11 | 6 | 5.09 | 1.38 | 1.38 | KIAA0232 | | | KIAA0232 gene product |
| PGD | U30255_at | U30255 | PASS | 6 | 22.00 | PASS | 10 | 6 | 16.00 | 1.38 | 1.38 | PGD | 1p36.3-p36.13 | phosphogluconate | phosphogluconate |
| | | | | | | | | | | | | | | dehydrogenase | dehydrogenase |
| EMAPII | U10117_at | U10117 | PASS | 9 | 10.11 | PASS | 11 | 9 | 7.36 | 1.37 | 1.37 | EMAPH | | endothelial-monocyte | endothelial-monocyte |
| | | | | | | | | | | | | | | activating polypeptide II | activating polypeptide II |
| DSS1 | U41515_at | U41515 | PASS | 9 | 8.78 | PASS | 10 | 9 | 6.40 | 1.37 | 1.37 | DSSI | | | Method: conceptual |
| | | | | | | | | | | | | | | | translation supplied by |
| | | | | | | | | | | | | | | | author. |
| M14199_s_at | M14199_s_at | M14199 | PASS | 9 | 302.00 | PASS | 13 | 9 | 220.23 | 1.37 | 1.37 | LAMR1 | 3p21.3 | laminin receptor 1 (67 kD); | laminin receptor 1 (67 kD); |
| | | | | | | | | | | | | | | Ribosomal protein SA | Ribosomal protein SA |
| STXBP3 | D63506_at | D63506 | PASS | 9 | 10.33 | PASS | 13 | 9 | 7.54 | 1.37 | 1.37 | Munc-18-3 | | unc-18 homologue | |
| NME1 | X17620_at | X17620 | PASS | 8 | 8.38 | PASS | 9 | 8 | 6.11 | 1.37 | 13.7 | NME1 | 17q22 | non-metastatic cells 1, | non-metastatic cells 1, |
| | | | | | | | | | | | | | | protein (NM23A) expressed | protein (NM23A) expressed |
| | | | | | | | | | | | | | | in | in |
| RPL3 | X73460_at | X73460 | PASS | 9 | 364.22 | PASS | 13 | 9 | 265.77 | 1.37 | 1.37 | RPL3 | 22 | ribosomal protein L3 | ribosomal protein L3 |
| APEX | D13370_at | D13370 | PASS | 9 | 20.44 | PASS | 13 | 9 | 14.92 | 1.37 | 1.37 | APEX | 14q11.2-q12 | APEX nuclease (multi- | APEX nuclease (multi- |
| | | | | | | | | | | | | | | functional DNA repair | functional DNA repair |
| | | | | | | | | | | | | | | | enzyme) |
| K201_HSP10 | D86956_at | D86956 | PASS | 6 | 7.00 | PASS | 9 | 6 | 5.11 | 1.37 | 13.7 | KIAA0201 | | | KIAA0201 gene product |
| COX10_rna1 | U82010_rna1 | U82010 | PASS | 9 | 8.56 | PASS | 12 | 9 | 6.25 | 1.37 | 1.37 | COX10 | 17p12-p11.2 | cytochrome c oxidase | cytochrome c oxidase |
| | | | | | | | | | | | | | | subunit X (heme A | subunit X (heme A: |
| | | | | | | | | | | | | | | | farnesyltransferase) |
| LYZ_f | M19045_f_at | M19045 | PASS | 9 | 155.67 | PASS | 13 | 9 | 113.77 | 1.37 | 1.37 | LYZ | | | lysozyme precursor (EC |
| | | | | | | | | | | | | | | | 3 2.1.17) |
| RPL19 | X63527_at | X63527 | PASS | 9 | 379.44 | PASS | 13 | 9 | 277.54 | 1.37 | 1.37 | RPL19 | 17p12-q11 | ribosomal protein L19 | ribosomal protein L19 |
| DPYD | U20938_at | U20938 | PASS | 8 | 9.25 | PASS | 13 | 8 | 6.77 | 1.37 | 1.37 | | | dihydropyrimidine | DPD; dihydrouracil |
| | | | | | | | | | | | | | | dehydrogenase | dehydrogenase |
| Z47055_s_at | Z47055_s_at | Z47055 | PASS | 9 | 9.22 | PASS | 12 | 9 | 6.75 | 1.37 | 1.37 | FDPS | | farnesyl diphosphate | farnesyl diphosphate |
| | | | | | | | | | | | | | | synthase (farnesyl pyro- | synthase (farnesyl pyro- |
| | | | | | | | | | | | | | | phosphate synthetase, | phosphate synthetase, |
| | | | | | | | | | | | | | | dimethylallyltransferase, | dimethylallyltransferase, |
| | | | | | | | | | | | | | | geranyltranstransferase) | geranyltranstransferase) |
| LYN | M16038_at | M16038 | PASS | 9 | 17.11 | PASS | 13 | 9 | 12.54 | 1.36 | 1.36 | LYN | 8q13 | v-yes-1 Yamaguchi | v-yes-1 Yamaguchi |
| | | | | | | | | | | | | | | sarcoma viral related | sarcoma viral related |
| | | | | | | | | | | | | | | oncogene homolog | oncogene homolog |
| hum_ala_at | hum_ala_at | hum | PASS | 9 | 422.44 | PASS | 13 | 9 | 309.62 | 1.36 | 1.36 | | | | |
| PCM1 | L27841_at | L27841 | PASS | 6 | 7.83 | PASS | 8 | 6 | 5.75 | 1.36 | 1.36 | PCM-1 | | pericentriol material 1 | |
| ACAA | D16294_at | D16294 | PASS | 8 | 9.13 | PASS | 10 | 8 | 6.70 | 1.36 | 1.36 | | | mitochondrial 3-oxoacyl- | |
| | | | | | | | | | | | | | | CoA thiolase | |
| PSMA3 | D00760_at | D00760 | PASS | 8 | 11.00 | PASS | 12 | 8 | 8.08 | 1.36 | 1.36 | PSMA2 | 6q27 | proteasome (prosome, | proteasome, (prosome, |
| | | | | | | | | | | | | | | macropain) subunit, alpha | macropain) subunit, alpha |
| | | | | | | | | | | | | | | type, 2 | type, 2 |
| U07806_s_at | U07806_s_at | U07806 | PASS | 8 | 11.25 | PASS | 11 | 8 | 8.27 | 1.36 | 1.36 | | | DNA topoisomerase I | found in the camptothecin |
| | | | | | | | | | | | | | | | resistant clone CEM/C2 |
| A82KD | U15552_at | U15552 | PASS | 7 | 8.71 | PASS | 12 | 7 | 6.42 | 1.36 | 1.36 | | | acidic 82 kDa protein | |
| D26535_s_at | D26535_s_at | D26535 | PASS | 8 | 10.00 | PASS | 11 | 8 | 7.36 | 1.36 | 1.36 | DLST | 14q24.3 | dihydrolipoamide S- | dihydrolipoamide S- |
| | | | | | | | | | | | | | | succinyltransferase (E2- | succinyltransferase (E2- |
| | | | | | | | | | | | | | | oxo-glutarate complex) | oxo-glutarate complex) |
| Z49148_s_at | 49148_s_at | Z49148 | PASS | 9 | 285.33 | PASS | 13 | 9 | 210.23 | 1.36 | 1.36 | RPL29 | 3q29-qter | ribosomal protein L29 | ribosomal protein L29 |
| EED | U90651_at | U90651 | PASS | 7 | 8.14 | PASS | 11 | 7 | 6.00 | 1.36 | 1.36 | EED | 11q14.2-q22.3 | embryonic ectoderm | embryonic ectoderm |
| | | | | | | | | | | | | | | development protein | development protein |
| | | | | | | | | | | | | | | homolog | homolog |
| DYRK2 | Y09216_at | Y09216 | PASS | 6 | 10.17 | PASS | 12 | 6 | 7.50 | 1.36 | 1.36 | DYRK2 | 12 | dual-specificity tyrosine- | dual-specificity tyrosine- |
| | | | | | | | | | | | | | | (Y)-phosphorylation | (Y)-phosphorylation |
| | | | | | | | | | | | | | | regulated kinase 2 | regulated kinase 2 |
| LLREP3 | X17206_at | X17206 | PASS | 9 | 471.00 | PASS | 13 | 9 | 347.46 | 1.36 | 1.36 | RPS2 | 16p13.3 | ribosomal protein S2 | ribosomal protein S2 |
| UBA52_rna1 | X56997_rna1 | X56997 | PASS | 9 | 268.44 | PASS | 13 | 9 | 198.08 | 1.36 | 1.36 | UbA52 | | ubiquitin-52 amino acid | |
| | | | | | | | | | | | | | | fusion protein | |
| RPS18 | X69150_at | X69150 | PASS | 9 | 377.78 | PASS | 13 | 9 | 278.77 | 1.36 | 1.36 | PRS18 | 6p21.3 | | ribosomal protein S18 |
| FCER2 | M15059_at | M15059 | PASS | 9 | 9.33 | PASS | 9 | 9 | 6.89 | 1.35 | 1.35 | FCER2 | 19p13.3 | Fc fragment of IgE, low | Fc fragment of IgE, low |
| | | | | | | | | | | | | | | affinity II, receptor for | affinity II, receptor for |
| | | | | | | | | | | | | | | (CD23A) | (CD23A) |
| P87 | L42572_at | L42572 | PASS | 8 | 8.38 | PASS | 11 | 8 | 6.18 | 1.35 | 1.35 | p87/89 | | transmembrane protein | endoplasmic reticulum |
| | | | | | | | | | | | | | | | protein |
| BTK_rna4 | U78027_rna4 | U78027 | PASS | 7 | 10.71 | PASS | 12 | 7 | 7.92 | 1.35 | 1.35 | FTP3 | | FTP3 | lysosomal exoglycosidase |
| HCG8 | X92110_at | X92110 | PASS | 5 | 5.80 | PASS | 7 | 5 | 4.29 | 1.35 | 1.35 | | | | |
| PSKH1 | U09564_at | U09564 | PASS | 8 | 7.50 | PASS | 11 | 8 | 5.55 | 1.35 | 1.35 | SRPK1 | 6p21.2-p21.3 | SFRS protein kinase 1 | SFRS protein kinase 1 |
| U09820_s_at | U09820_s_at | U09820 | PASS | 7 | 8.71 | PASS | 9 | 7 | 6.44 | 1.35 | 1.35 | ATRX | Xq13.1-q21.1 | helicase II | alpha thalessemia/mental |
| | | | | | | | | | | | | | | | retardation syndrome |
| | | | | | | | | | | | | | | | X-linked |
| U37546_s_at | U37546_s_at | U37546 | PASS | 8 | 7.13 | PASS | 11 | 8 | 5.27 | 1.35 | 1.35 | API1 | 11q22 | apoptosis inhibitor 1 | apoptosis inhibitor 1 |
| S54005_s_at | S54005_s_at | S54005 | PASS | 9 | 184.44 | PASS | 13 | 9 | 136.54 | 1.35 | 1.35 | thymosin | | | |
| | | | | | | | | | | | | beta-10 | | | |
| LKYHYD | U57721_at | U57721 | PASS | 9 | 9.44 | PASS | 13 | 9 | 7.00 | 1.35 | 1.35 | KYNU | | kynureninase; 1-kynurenine | kynureninase; 1-kynurenine |
| | | | | | | | | | | | | | | hydrolase | hydrolase |
| X95325_s_at | X95325_s_at | X95325 | PASS | 5 | 21.80 | PASS | 12 | 5 | 16.17 | 1.35 | 1.35 | dpbAv | | DNA-binding protein | variant A |
| RARS | S80343_at | S80343 | PASS | 8 | 7.50 | PASS | 7 | 8 | 5.57 | 1.35 | 1.35 | RARS | 5pter-q11 | arginyl-tRNA synthetase | arginyl-tRNA synthetase |
| K148 | D63482_at | D63482 | PASS | 5 | 7.80 | PASS | 10 | 5 | 5.80 | 1.34 | 1.34 | KIAA0148 | | | KIAA0148 gene product |
| K178_SMC1 | D80000_at | D80000 | PASS | 6 | 6.17 | PASS | 10 | 6 | 4.60 | 1.34 | 1.34 | KIAA0178 | | | similar to mitosis-specific |
| | | | | | | | | | | | | | | | chromosome segregation |
| | | | | | | | | | | | | | | | protein SMC1 of S. |
| | | | | | | | | | | | | | | | cerevisiae. |
| RPS11 | X06617_at | X06617 | PASS | 9 | 380.22 | PASS | 13 | 9 | 284.08 | 1.34 | 1.34 | RPS11 | 19q13.3 | ribosomal protein S11 | ribosomal protein S11 |
| JUN | U65928_at | U65928 | PASS | 8 | 8.75 | PASS | 11 | 8 | 6.55 | 1.34 | 1.34 | JUN | 1p32-p31 | Avian sarcoma virus 17 | v-jun avian sarcoma virus |
| | | | | | | | | | | | | | | (v-jun) oncogene homolog | 17 oncogene homolog |
| INT6 | U62962_at | U62962 | PASS | 9 | 52.67 | PASS | 13 | 9 | 39.46 | 1.33 | 1.33 | EIF3S6 | 8q22-q23 | murine mammary tumor | eukaryotic translation |
| | | | | | | | | | | | | | | integration site 6 | initiation factor 3, |
| | | | | | | | | | | | | | | (oncogene homolog) | subunit 6 (48 kD) |
| RSU1 | L12535_at | L12535 | PASS | 9 | 13.44 | PASS | 13 | 9 | 10.08 | 1.33 | 1.33 | RSU-1 | | | homologous to mouse |
| | | | | | | | | | | | | | | | Rsu-1; putative |
| SFRS7_rna1 | L41887_rna1 | L41887 | PASS | 6 | 6.33 | PASS | 8 | 6 | 4.75 | 1.33 | 1.33 | SFRS7 | | splicing factor, arginine/ | 35 kDa protein |
| | | | | | | | | | | | | | | serine-rich 7 | |
| K71 | D31888_at | D31888 | PASS | 8 | 7.25 | PASS | 9 | 8 | 5.44 | 1.33 | 1.33 | KIAA0071 | | | |
| ANT3 | J03592_at | J03592 | PASS | 9 | 200.78 | PASS | 13 | 9 | 151.00 | 1.33 | 1.33 | ANT3 | Xp22.32 | | adenine nucleotide trans- |
| | | | | | | | | | | | | | | | locator 3 (liver) |
| MDH1 | D55654_at | D55654 | PASS | 9 | 25.56 | PASS | 13 | 9 | 19.23 | 1.33 | 1.33 | | | cytosolic malate | |
| | | | | | | | | | | | | | | dehydrogenase | |
| RPLP2 | AB002533_at | AB002533 | PASS | 9 | 355.11 | PASS | 13 | 9 | 267.23 | 1.33 | 1.33 | KPNA4 | | karyopherin alpha 4 | karyopherin alpha 4 |
| | | | | | | | | | | | | | | (importin alpha 3) | (importin alpha 3) |
| PSMA5 | D00761_at | D00761 | PASS | 9 | 33.11 | PASS | 13 | 9 | 24.92 | 1.33 | 1.33 | PSMB1 | 7p13-p12 | proteasome (prosome, | proteasome (prosome, |
| | | | | | | | | | | | | | | macropain) subunit, beta | macropain) subunit, beta |
| | | | | | | | | | | | | | | type, 1 | type, 1 |
| NPM1 | M23613_at | M23613 | PASS | 9 | 103.33 | PASS | 13 | 9 | 77.85 | 1.33 | 1.33 | NPM1 | 5q35 | nucleophosmin | nucleophosmin (nuclear |
| | | | | | | | | | | | | | | | phosphoprotein B23, |
| | | | | | | | | | | | | | | | numatrin) |
| K2_TRIC5 | D13627_at | D13627 | PASS | 9 | 15.78 | PASS | 12 | 9 | 11.92 | 1.32 | 1.32 | KIAA0002 | | | KIAA0002 gene product |
| GZMK | U26174_at | U26174 | PASS | 6 | 17.50 | PASS | 13 | 6 | 13.23 | 1.32 | 1.32 | GZMK | | granzyme K, (serine | granzyme K (serine |
| | | | | | | | | | | | | | | protease, granzyme 3; | protease, granzyme 3; |
| | | | | | | | | | | | | | | tryptase II) | tryptase II) |
| MRP | X78338_at | X78338 | PASS | 7 | 7.43 | PASS | 8 | 7 | 5.63 | 1.32 | 1.32 | | | | |
| M16336_s_at | M16336_s_at | M16336 | PASS | 9 | 52.11 | PASS | 13 | 9 | 39.54 | 1.32 | 1.32 | CD2 | 1p13 | CD2 antigen (p50), sheep | CD2 antigen (p50), sheep |
| | | | | | | | | | | | | | | red blood cell receptor | red blood cell receptor |
| K164_DNAB | D79986_at | D79986 | PASS | 8 | 16.63 | PASS | 13 | 8 | 12.62 | 1.32 | 1.32 | KIAA0164 | | | KIAA0164 gene product |
| RPL17 | X53777_at | X53777 | PASS | 9 | 206.44 | PASS | 13 | 9 | 156.69 | 1.32 | 1.32 | RPL17 | 18q | ribosomal protein L17 | ribosomal protein L17 |
| SPHAR_rna1 | X82554_rna1 | X82554 | PASS | 5 | 6.00 | PASS | 9 | 5 | 4.56 | 1.32 | 1.32 | SPHAR | | | |
| SRC1 | U59302_at | U59302 | PASS | 9 | 11.44 | PASS | 13 | 9 | 8.69 | 1.32 | 1.32 | SRC1 | 2p23 | steroid receptor coactivator | steroid receptor coactivator |
| | | | | | | | | | | | | | | 1 | 1 |
| FLI1 | M98833_at | M98833 | PASS | 7 | 8.29 | PASS | 13 | 7 | 6.31 | 1.31 | 1.31 | FLI1 | 11q24.1-q24.3 | Friend leukemia virus | Friend leukemia virus |
| | | | | | | | | | | | | | | integration 1 | integration 1 |
| K16 | D13641_at | D13641 | PASS | 8 | 13.13 | PASS | 13 | 8 | 10.00 | 1.31 | 1.31 | KIAA0016 | | mitochondrial outer | KIAA0016 gene product |
| | | | | | | | | | | | | | | membrane protein 19 | |
| TFAP3S | U91932_at | U91932 | PASS | 9 | 14.33 | PASS | 13 | 9 | 10.92 | 1.31 | 1.31 | CLAPS3 | 12p13.2-p13.1 | clathrin-associated/ | clathrin-associated/ |
| | | | | | | | | | | | | | | assembly/adaptor protein, | assembly/adaptor protein, |
| | | | | | | | | | | | | | | small 3 (22 kD) | small 3, 22-kD; Sigma3 A |
| GUA5MPST | HG4716-HT5 | HG4716-HT | PASS | 8 | 7.75 | PASS | 11 | 8 | 5.91 | 1.31 | 1.31 | | | | |
| RPS28 | U58682_at | U58682 | PASS | 9 | 286.67 | PASS | 13 | 9 | 218.69 | 1.31 | 1.31 | RPS28 | 19p13.2 | ribosomal protein S28 | ribosomal protein S28 |
| RAB11A | AF000231_at | AF000231 | PASS | 6 | 6.17 | PASS | 7 | 6 | 4.71 | 1.31 | 1.31 | | | rab11a | GTPase |
| AFFX-HUM | AFFX-HUM | AFFX-HUM | PASS | 6 | 12.17 | PASS | 13 | 6 | 9.31 | 1.31 | 1.31 | | | | |
| HPRT1 | M31642_at | M31642 | PASS | 8 | 7.63 | PASS | 12 | 8 | 5.83 | 1.31 | 1.31 | HPRT1 | Xq26.1 | hypoxanthine phosphori- | hypoxanthine phosphori- |
| | | | | | | | | | | | | | | bosyltransferase 1 | bosyltransferase 1 |
| | | | | | | | | | | | | | | (Lesch-Nyhan syndrome) | (Lesch-Nyhan syndrome) |
| PSM112 | D44466_at | D44466 | PASS | 9 | 7.44 | PASS | 10 | 9 | 5.70 | 1.31 | 1.31 | PSMD1 | | proteasome (prosome, | proteasome (prosome, |
| | | | | | | | | | | | | | | macropain) 26S subunit, | macropain) 26S subunit, |
| | | | | | | | | | | | | | | non-ATPase, 1 | non-ATPase, 1 |
| PEBP2AC1 | Z35278_at | Z35278 | PASS | 9 | 19.78 | PASS | 13 | 9 | 15.15 | 1.31 | 1.31 | CBFA3 | 1p36 | core-binding factor, | core-binding factor, |
| | | | | | | | | | | | | | | runt domain, alpha | runt domain, alpha |
| | | | | | | | | | | | | | | subunit 3 | subunit 3 |
| PSMCP31 | D38047_at | D38047 | PASS | 9 | 31.22 | PASS | 13 | 9 | 23.92 | 1.31 | 1.31 | PSMD8 | | proteasome (prosome, | proteasome (prosome, |
| | | | | | | | | | | | | | | macropain) 26S subunit, | macropain) 26S subunit, |
| | | | | | | | | | | | | | | non-ATPase, 8 | non-ATPase, 8 |
| PSM42 | D78275_at | D78275 | PASS | 8 | 9.25 | PASS | 10 | 8 | 7.10 | 1.30 | 1.30 | PSMC6 | 12q15 | proteasome (prosome, | proteasome (prosome, |
| | | | | | | | | | | | | | | macropain) 26S subunit, | macropain) 26S subunit, |
| | | | | | | | | | | | | | | ATPase, 6 | ATPase, 6 |
| P76 | U81006_at | U81006 | PASS | 7 | 9.00 | PASS | 11 | 7 | 6.91 | 1.30 | 1.30 | P76 | | 76 kDa membrane protein | 76 kDa membrane protein |
| K54_MOV10 | D29677_at | D29677 | PASS | 7 | 8.29 | PASS | 11 | 7 | 6.36 | 1.30 | 1.30 | KIAA0054 | | | |
| SKIP | U51432_at | U51432 | PASS | 6 | 13.00 | PASS | 10 | 6 | 10.00 | 1.30 | 1.30 | | | nuclear protein Skip | similar to the Drosophila |
| | | | | | | | | | | | | | | | puff specific protein Bx42 |
| K276 | D87445_at | D87445 | PASS | 6 | 6.50 | PASS | 9 | 6 | 5.00 | 1.30 | 1.30 | KIAA0256 | | | KIAA0256 gene product |
| LGALS8 | L78132_at | L78132 | PASS | 6 | 6.17 | PASS | 8 | 6 | 4.75 | 1.30 | 1.30 | peta-1 | | prostate carcinoma tumor | |
| | | | | | | | | | | | | | | antigen | |
| POLR2 | U37689_at | U37689 | PASS | 5 | 9.80 | PASS | 9 | 5 | 7.56 | 1.30 | 1.30 | hsRPB8 | | RNA polymerase II subunit | |
| CASP10 | U60519_at | U60519 | PASS | 6 | 6.67 | PASS | 7 | 6 | 5.14 | 1.30 | 1.30 | CASP10 | 2q33-q34 | caspase 10, apoptosis- | caspase 10, apoptosis- |
| | | | | | | | | | | | | | | related cysteine protease | related cysteine protease |
| L14778_s_at | L14778_{'}s_at | L14778 | PASS | 9 | 9.78 | PASS | 11 | 9 | 7.55 | 1.30 | 1.30 | PPP3CA | 4q21-q24 | calmodulin-dependent | protein phosphatase 3 |
| | | | | | | | | | | | | | | phosphatase catalytic | (formerly 2B), catalytic |
| | | | | | | | | | | | | | | subunit | subunit, alpha isoform |
| | | | | | | | | | | | | | | | (calcineurin A alpha) |
| RAB_rna1 | L42025_rna1 | L42025 | PASS | 6 | 6.33 | PASS | 9 | 6 | 4.89 | 1.30 | 1.30 | HRB | 2q36 | HIV-1 Rev binding protein | HIV-1 Rev binding protein |
| GAPDHM | AFFX-HUM | AFFX-HUM | PASS | 9 | 162.00 | PASS | 13 | 9 | 125.31 | 1.29 | 1.29 | | | | |
| E_121711DM | U92014_at | U92014 | PASS | 6 | 6.83 | PASS | 7 | 6 | 5.29 | 1.29 | 1.29 | | | | |
| HRMT1L1 | X99209_at | X99209 | PASS | 9 | 20.78 | PASS | 13 | 9 | 16.08 | 1.29 | 1.29 | | | arginine methyltransferase | |
| RPL10 | HG4542-HT4 | HG4542-HT | PASS | 9 | 184.78 | PASS | 13 | 9 | 143.08 | 1.29 | 1.29 | | | | |
| 28SRNA5 | AFFX-M2783 | AFFX-M27 | PASS | 7 | 8.71 | PASS | 8 | 7 | 6.57 | 1.29 | 1.29 | | | | |
| YWHA | X56468_at | X56468 | PASS | 9 | 23.33 | PASS | 13 | 9 | 18.08 | 1.29 | 1.29 | | | 14.3.3 protein | |
| ACADM | M91432_at | M91432 | PASS | 8 | 7.50 | PASS | 11 | 8 | 5.82 | 1.29 | 1.29 | ACADM | 1p31 | acyl-Coenzyme A | acyl-Coenzyme A |
| | | | | | | | | | | | | | | dehydrogenase, C-4 to C-12 | dehydrogenase, C-4 to C-12 |
| | | | | | | | | | | | | | | straight chain | straight chain |
| FMR1 | U25165_at | U25165 | PASS | 7 | 11.00 | PASS | 13 | 7 | 8.54 | 1.29 | 1.29 | FXR1 | 3q28 | FXR1 | fragile X mental |
| | | | | | | | | | | | | | | | retardation, autosomal |
| | | | | | | | | | | | | | | | homolog 1 |
| HMG17_rna1 | X13546_rna1 | X13546 | PASS | 9 | 40.22 | PASS | 13 | 9 | 31.23 | 1.29 | 1.29 | HMG17 | 1p36.1-p35 | put HMG-17 protein | high-mobility group |
| | | | | | | | | | | | | | | | (nonhistone chromosomal) |
| | | | | | | | | | | | | | | | protein 17 |
| K6_VAV1 | D25304_at | D25304 | PASS | 9 | 16.11 | PASS | 13 | 9 | 12.54 | 1.28 | 1.28 | KIAA0006 | | | PAK-interacting exchange |
| | | | | | | | | | | | | | | | factor alpha |
| SNRPD2 | U15008_at | U15008 | PASS | 9 | 140.33 | PASS | 13 | 9 | 109.23 | 1.28 | 1.28 | SNRPD2 | | small nuclear ribonucleo- | small nuclear ribonucleo- |
| | | | | | | | | | | | | | | protein D2 polypeptide | protein D2 polypeptide |
| | | | | | | | | | | | | | | (16.5 kD) | (16.5 kD) |
| COX5A | M22760_at | M22760 | PASS | 9 | 16.89 | PASS | 13 | 9 | 13.15 | 1.28 | 1.28 | COX5A | 15q25 | cytochrome c oxidase | cytochrome c oxidase |
| | | | | | | | | | | | | | | subunit Va | subunit Va |
| VRK1 | AB000449_at | AB000449 | PASS | 7 | 7.57 | PASS | 10 | 7 | 5.90 | 1.28 | 1.28 | VRK1 | 14q32 | vaccinia related kinase 1 | vaccinia related kinase 1 |
| M31516_s_at | M31516_s_at | M31516 | PASS | 5 | 6.20 | PASS | 12 | 5 | 4.83 | 1.28 | 1.28 | DAF | 1q32 | decay-accelerating factor | decay accelerating factor |
| | | | | | | | | | | | | | | | for complement (CD55, |
| | | | | | | | | | | | | | | | Cromer blood group |
| | | | | | | | | | | | | | | | system) |
| TRPOSL | M23161_at | M23161 | PASS | 7 | 6.29 | PASS | 11 | 7 | 4.91 | 1.28 | 1.28 | | | | |
| PSMC5 | L38810_at | L38810 | PASS | 9 | 14.67 | PASS | 11 | 9 | 11.45 | 1.28 | 1.28 | PSMC5 | 17q23-q25 | proteasome (prosome, | proteasome (prosome, |
| | | | | | | | | | | | | | | macropain) 26S subunit, | macropain) 26S subunit, |
| | | | | | | | | | | | | | | ATPase, 5 | ATPase, 5 |
| UROD | M14016_at | M14016 | PASS | 7 | 7.86 | PASS | 7 | 7 | 6.14 | 1.28 | 1.28 | UROD | 1p34 | uroporphyrinogen | uroporphyrinogen |
| | | | | | | | | | | | | | | decarboxylase | decarboxylase |
| POLR2 | HG2274-HT2 | HG2274-HT | PASS | 6 | 10.33 | PASS | 12 | 6 | 8.08 | 1.28 | 1.28 | | | | |
| M96954_s_at | M96954_s_at | M96954 | PASS | 9 | 7.67 | PASS | 13 | 9 | 6.00 | 1.28 | 1.28 | | | Nuclelysin TIAR | |
| TRP185 | U38847_at | U38847 | PASS | 5 | 6.20 | PASS | 7 | 5 | 4.86 | 1.28 | 1.28 | TRP-185 | | TAR RNA loop binding | TRP-185 |
| | | | | | | | | | | | | | | protein; | |
| GCNT1 | U77413_at | U77413 | PASS | 5 | 7.40 | PASS | 10 | 5 | 5.80 | 1.28 | 1.28 | OGT | | O-GlcNAc transferase | O-linked N-acetyl- |
| | | | | | | | | | | | | | | (uridine diphospho-N- | glucosamine (GlcNAc) |
| | | | | | | | | | | | | | | acetylglucosamine:poly- | transferase (UDP-N- |
| | | | | | | | | | | | | | | peptide beta-N-acetyl- | acetylglucosamine:poly- |
| | | | | | | | | | | | | | | glucosaminyl transferase) | peptide-N-acetyl- |
| | | | | | | | | | | | | | | | glucosaminyl transferase) |
| RAN | HG1112-HT1 | HG1112-HT | PASS | 9 | 21.44 | PASS | 13 | 9 | 16.85 | 1.27 | 1.27 | | | | |
| INDPOLABP | U33818_at | U33818 | PASS | 8 | 14.88 | PASS | 13 | 8 | 11.69 | 1.27 | 1.27 | IPABP | | inducible poly(A)-binding | inducible poly(A)-binding |
| | | | | | | | | | | | | | | protein | protein |
| DPM1 | AF007875_at | AF007875 | PASS | 8 | 8.38 | PASS | 12 | 8 | 6.58 | 1.27 | 1.27 | DPM1 | | dolichyl-phosphate | dolichyl-phosphate |
| | | | | | | | | | | | | | | mannosyltransferase poly- | mannosyltransferase poly- |
| | | | | | | | | | | | | | | peptide 1, catalytic subunit | peptide 1, catalytic subunit |
| TCP3 | M31523_{'}at | M31523 | PASS | 9 | 6.89 | PASS | 12 | 9 | 5.42 | 1.27 | 1.27 | TCF3 | 19 | | transcription factor 3 |
| | | | | | | | | | | | | | | | (E2A immunoglobulin |
| | | | | | | | | | | | | | | | enhancer binding factors |
| | | | | | | | | | | | | | | | E12/E47) |
| Z26491_s_at | Z26491_s_at | Z26491 | PASS | 9 | 13.89 | PASS | 13 | 9 | 10.92 | 1.27 | 1.27 | | | catechol O-methyl- | |
| | | | | | | | | | | | | | | transferase | |
| HNRNPCL | M94630_at | M94630 | PASS | 9 | 27.56 | PASS | 13 | 9 | 21.69 | 1.27 | 1.27 | HNRPD | 4q21 | heterogeneous nuclear | heterogeneous nuclear |
| | | | | | | | | | | | | | | ribonucleoprotein D | ribonucleoprotein D |
| K212_COSC | D86967_at | D86967 | PASS | 7 | 11.43 | PASS | 12 | 7 | 9.00 | 1.27 | 1.27 | KIAA0212 | | | KIAA0212 gene product |
| EIF2A | U26032_at | U26032 | PASS | 5 | 5.80 | PASS | 7 | 5 | 4.57 | 1.27 | 1.27 | | | | |
| TGFBR2 | D50683_at | D50683 | PASS | 9 | 24.00 | PASS | 13 | 9 | 18.92 | 1.27 | 1.27 | TGFBR2 | 3p22 | transforming growth factor, | transforming growth factor, |
| | | | | | | | | | | | | | | beta receptor II (70-80 kD) | beta receptor II (70-80 kD) |
| PPP2R2A | M64929_at | M64929 | PASS | 7 | 7.29 | PASS | 8 | 7 | 5.75 | 1.27 | 1.27 | PPP2R2A | | protein phosphatase 2 | protein phosphatase 2 |
| | | | | | | | | | | | | | | (formerly 2A), regulatory | (formerly 2A), regulatory |
| | | | | | | | | | | | | | | subunit B (PR 52), alpha | subunit B (PR 52), alpha |
| | | | | | | | | | | | | | | isoform | isoform |
| GPRK5 | L15388_at | L15388 | PASS | 6 | 6.33 | PASS | 7 | 6 | 5.00 | 1.27 | 1.27 | GPRK5 | 10q24-qter | G protein-coupled | G protein-coupled |
| | | | | | | | | | | | | | | receptor kinase | receptor kinase |
| PPP3CB2 | M29551_at | M29551 | PASS | 5 | 7.60 | PASS | 9 | 5 | 6.00 | 1.27 | 1.27 | | | | calcineurin A2 |
| HG3484-HT3 | HG3484-HT3 | HG3484-HT | PASS | 7 | 8.86 | PASS | 12 | 7 | 7.00 | 1.27 | 1.27 | | | | |
| CCNH | U11791_at | U11791 | PASS | 7 | 6.43 | PASS | 12 | 7 | 5.08 | 1.26 | 1.26 | CCNH | 5q13.3-q14 | cyclin H | cyclin H |
| H2B_rna2 | X57985_rna2 | X57985 | PASS | 9 | 9.11 | PASS | 13 | 9 | 7.23 | 1.26 | 1.26 | H2AFQ | 1q21-q23 | histone H2A | H2A histone family, |
| | | | | | | | | | | | | | | | member Q |
| NFKB1 | M58603_at | M58603 | PASS | 6 | 16.17 | PASS | 12 | 6 | 12.83 | 1.26 | 1.26 | NFKB1 | 4q24 | nuclear factor kappa-B | nuclear factor of kappa |
| | | | | | | | | | | | | | | DNA binding subunit | light polypeptide gene |
| | | | | | | | | | | | | | | | enhancer in B-cells 1 |
| | | | | | | | | | | | | | | | (p105) |
| G22P1 | J04611_at | J04611 | PASS | 9 | 23.44 | PASS | 13 | 9 | 18.62 | 1.26 | 1.26 | G22P1 | 22q11-q13 | thyroid autoantigen 70 kD | thyroid autoantigen 70 kD |
| | | | | | | | | | | | | | | (Ku antigen) | (Ku antigen) |
| RABGGTB | X98001_at | X98001 | PASS | 6 | 6.67 | PASS | 10 | 6 | 5.30 | 1.26 | 1.26 | RABGGTB | 1p31-p22 | Rab geranylgeranyl- | Rab geranylgeranyl- |
| | | | | | | | | | | | | | | transferase, beta subunit | transferase, beta subunit |
| ECH1 | U16660_at | U16660 | PASS | 7 | 20.57 | PASS | 13 | 7 | 16.38 | 1.26 | 1.26 | ECH1 | 19q13.1 | enoyl Coenzyme A | enoyl Coenzyme A |
| | | | | | | | | | | | | | | hydratase 1, peroximal | hydratase 1, peroximal |
| K276_HYPLK | D87466_at | D87466 | PASS | 7 | 5.71 | PASS | 9 | 7 | 4.56 | 1.25 | 1.25 | KIAA0276 | | | Similar to S. cerevisiae |
| | | | | | | | | | | | | | | | hypothetical protein L3111 |
| | | | | | | | | | | | | | | | (S59316) |
| K78_RAD21 | D38551_at | D38551 | PASS | 8 | 10.63 | PASS | 12 | 8 | 8.50 | 1.25 | 1.25 | RAD21 | | | RAD21 (S. pombe) |
| | | | | | | | | | | | | | | | homolog |
| ECGF1 | M31210_at | M31210 | PASS | 8 | 6.38 | PASS | 10 | 8 | 5.10 | 1.25 | 1.25 | EDG1 | 1pter-qter | endothelial differentiation, | endothelial differentiation, |
| | | | | | | | | | | | | | | sphingolipid G-protein- | sphingolipid G-protein- |
| | | | | | | | | | | | | | | coupled receptor, 1 | coupled receptor, 1 |
| M58525_s_at | M58525_s_at | M58525 | PASS | 5 | 10.40 | PASS | 12 | 5 | 8.33 | 1.25 | 1.25 | COMT | 22q11.21- | catechol-O-methyl- | catechol-O-methyl- |
| | | | | | | | | | | | | | | transferase | transferase |
| HG2639-HT2 | HG2639-HT2 | HG2639-HT | PASS | 9 | 11.89 | PASS | 13 | 9 | 95.4 | 1.25 | 1.25 | | | | |
| ZNF43_f | X59244_f_at | X59244 | PASS | 5 | 5.60 | PASS | 10 | 5 | 4.50 | 1.24 | 1.24 | ZNF43 | 19p13.1-p12 | zinc finger protein 43 | zinc finger protein 43 |
| | | | | | | | | | | | | | | (HTF6) | (HTF6) |
| D79984_s_at | D79984_s_at | D79984 | PASS | 5 | 6.40 | PASS | 7 | 5 | 5.14 | 1.24 | 1.24 | KIAA0162 | | | similar to emb-5 protein |
| | | | | | | | | | | | | | | | of C. elegans. |
| MIF_rna1 | L19686_rna1 | L19686 | PASS | 9 | 43.33 | PASS | 13 | 9 | 34.85 | 1.24 | 1.24 | MIF | 22q11.2 | macrophage migration | macrophage migration |
| | | | | | | | | | | | | | | inhibitory factor (glcosyla- | inhibitory factor (glcosyla- |
| | | | | | | | | | | | | | | tion-inhibiting factor) | tion-inhibiting factor) |
| CBF | M37197_at | M37197 | PASS | 8 | 6.75 | PASS | 7 | 8 | 5.43 | 1.24 | 1.24 | CEBP | | CCAAT-box-binding factor | |
| M90391_s_at | M90391_s_at | M90391 | PASS | 7 | 7.71 | PASS | 9 | 7 | 6.22 | 1.24 | 1.24 | IL16 | | interleukin 16 (lymphocyte | interleukin 16 (lymphocyte |
| | | | | | | | | | | | | | | chemoattractant factor) | chemoattractant factor) |
| K29 | D21852_at | D21852 | PASS | 9 | 7.33 | PASS | 12 | 9 | 5.92 | 1.24 | 1.24 | KIAA0029 | | | |
| CTSS | M90696_at | M90696 | PASS | 8 | 12.38 | PASS | 11 | 8 | 10.00 | 1.24 | 1.24 | CTSS | 1q21 | cathepsin S | cathepsin S |
| X15673_s_at | X15673_s_at | X15673 | PASS | 5 | 9.40 | PASS | 10 | 5 | 7.60 | 1.24 | 1.24 | | | | |
| ERH | D85758_at | D85758 | PASS | 7 | 21.86 | PASS | 13 | 7 | 17.69 | 1.24 | 1.24 | ERH | 7q34 | enhancer of rudimentary | enhancer of rudimentary |
| | | | | | | | | | | | | | | (Drosophila) homolog | (Drosophila) homolog |
| HUM31 | U30521_at | U30521 | PASS | 5 | 6.00 | PASS | 7 | 5 | 4.86 | 1.24 | 1.24 | P311 | | P311 protein | P311 protein |
| K244_TCEA | D87685_at | D87685 | PASS | 8 | 5.63 | PASS | 9 | 8 | 4.56 | 1.23 | 1.23 | KIAA0244 | | | similar to human |
| | | | | | | | | | | | | | | | transcription factor TFHS |
| | | | | | | | | | | | | | | | (S34159). |
| PSM1131 | D88378_at | D88378 | PASS | 6 | 6.00 | PASS | 8 | 6 | 4.88 | 1.23 | 1.23 | | | proteasome inhibitor hP131 | |
| | | | | | | | | | | | | | | subunit | |
| SRI | M32886_at | M32886 | PASS | 8 | 7.00 | PASS | 13 | 8 | 5.69 | 1.23 | 1.23 | SR1 | 7 | sorcin | sorcin |
| PRKAR1A | M33336_at | M33336 | PASS | 7 | 25.71 | PASS | 13 | 7 | 20.92 | 1.23 | 1.23 | PRKAR1A | 17q23-q24 | protein kinase, cAMP- | protein kinase, cAMP- |
| | | | | | | | | | | | | | | dependent, regulatory, type | dependent, regulatory, type |
| | | | | | | | | | | | | | | I, alpha (tissue specific | 1, alpha (tissue specific |
| | | | | | | | | | | | | | | extingisher 1) | extinguisher 1) |
| AMD1 | M21154_at | M21154 | PASS | 8 | 8.50 | PASS | 12 | 8 | 6.92 | 1.23 | 1.23 | AMD1 | 6q21-q22 | S-adenosylmethionine | S-adenosylmethionine |
| | | | | | | | | | | | | | | decarboxylase 1 | decarboxylase 1 |
| HG884-HT88 | HG884-HT88 | HG884-HT | PASS | 5 | 6.00 | PASS | 9 | 5 | 4.89 | 1.23 | 1.23 | | | | |
| RAB5IP | S83364_at | S83364 | PASS | 7 | 11.86 | PASS | 12 | 7 | 9.67 | 1.23 | 1.23 | putative | | | This sequence comes from |
| | | | | | | | | | | | | Rab5- | | | FIG. 4. |
| | | | | | | | | | | | | interacting | | | |
| | | | | | | | | | | | | protein | | | |
| K35_NOPP1 | D21262_at | D21262 | PASS | 9 | 7.11 | PASS | 10 | 9 | 5.80 | 1.23 | 1.23 | P130 | | nucleolar phosphoprotein | nucleolar phosphoprotein |
| | | | | | | | | | | | | | | p130 | p130 |
| CD36 | Z32765_at | Z32765 | PASS | 8 | 26.88 | PASS | 13 | 8 | 21.92 | 1.23 | 1.23 | | | | |
| ETFA | J04058_at | J04058 | PASS | 7 | 6.57 | PASS | 11 | 7 | 5.36 | 1.23 | 1.23 | ETFA | 15q23-q25 | electron-transfer-flavo- | electron-transfer-flavo- |
| | | | | | | | | | | | | | | protein, alpha polypeptide | protein, alpha polypeptide |
| | | | | | | | | | | | | | | (glutaric aciduria II) | (glutaric aciduria II) |
| TCEA1 | M81601_at | M81601 | PASS | 8 | 18.75 | PASS | 13 | 8 | 15.31 | 1.22 | 1.22 | transcription | | transcription elongation | |
| | | | | | | | | | | | | elongation | factor SII | | |
| GSN | S65738_at | S65738 | PASS | 9 | 10.00 | PASS | 12 | 9 | 8.17 | 1.22 | 1.22 | actin depoly- | | actin depolymerizing factor | This sequence comes from |
| | | | | | | | | | | | | merizing | | | FIG. 1B, destrin; ADF |
| | | | | | | | | | | | | factor, | | | |
| | | | | | | | | | | | | destrin, ADF | | | |
| D78132_s_at | D78132_s_at | D78132 | PASS | 8 | 7.63 | PASS | 13 | 8 | 6.23 | 1.22 | 1.22 | Rheb | | ras-related GTP-binding | Ras homologue encriched |
| | | | | | | | | | | | | | | protein | in brain; similar to rat |
| | | | | | | | | | | | | | | | Rheb gene |
| ITPR2 | D26070_at | D26070 | PASS | 5 | 5.80 | PASS | 12 | 5 | 4.75 | 1.22 | 1.22 | ITPR1 | 3p26-p25 | human type 1 inositol | inositol 1,4,5-triphosphate |
| | | | | | | | | | | | | | | 1,4,5-trisphosphate receptor | receptor, type 1 |
| HG210-HT21 | HG210-HT21 | HG210-HT | PASS | 5 | 5.80 | PASS | 8 | 5 | 4.75 | 1.22 | 1.22 | | | | |
| U28488_s_at | U28488_s_at | U28488 | PASS | 5 | 7.20 | PASS | 10 | 5 | 5.90 | 1.22 | 1.22 | C3AR1 | | complement component 3a | complement component 3a |
| | | | | | | | | | | | | | | receptor 1 | receptor 1 |
| EGR1 | S81439_at | S81439 | PASS | 7 | 5.86 | PASS | 10 | 7 | 4.80 | 1.22 | 1.22 | TIEG | | | TGFB inducible early |
| | | | | | | | | | | | | | | | growth response |
| TSC22 | U35048_at | U35048 | PASS | 9 | 15.11 | PASS | 13 | 9 | 12.38 | 1.22 | 1.22 | TSC22 | TSC-22 protein | transforming growth factor | |
| | | | | | | | | | | | | | | | beta-stimulated protein |
| | | | | | | | | | | | | | | | TSC-22 |
| DCN | M14219_at | M14219 | PASS | 7 | 7.43 | PASS | 11 | 7 | 6.09 | 1.22 | 1.22 | DCN | 12q23 | decorin | decorin |
| U00947_s_at | U00947_s_at | U00947 | PASS | 9 | 88.89 | PASS | 13 | 9 | 72.92 | 1.22 | 1.22 | | | | |
| K155_COSB | D63875_at | D63875 | PASS | 8 | 7.88 | PASS | 13 | 8 | 6.46 | 1.22 | 1.22 | KIAA0155 | | | KIAA0155 gene product |
| X57152_rna1 | X57152_rna1 | X57152 | PASS | 9 | 28.00 | PASS | 13 | 9 | 23.00 | 1.22 | 1.22 | | | casein kinase II subunit | protein kinase |
| | | | | | | | | | | | | | | beta | |
| Y00451_s_at | Y00451_s_at | Y00451 | PASS | 8 | 5.75 | PASS | 11 | 8 | 4.73 | 1.22 | 1.22 | ALAS2 | Xp11.21 | aminolevulinate, delta-, | aminolevulinate, delta-, |
| | | | | | | | | | | | | | | synthase 2 *sideroblastic/ | synthase 2 (sideroblastic/ |
| | | | | | | | | | | | | | | hypochromic anemia) | hypochromic anemia) |
| HADHB | D16481_at | D16481 | PASS | 6 | 9.17 | PASS | 13 | 6 | 7.54 | 1.22 | 1.22 | HADHB | 2p23 | hydroxyacyl-Coenzyme A | hydroxyacyl-Coenzyme A |
| | | | | | | | | | | | | | | dehydrogenase/3-ketoacyl- | dehydrogenase/3-ketoacyl- |
| | | | | | | | | | | | | | | Coenzyme A thiolase/ | Coenzyme A thiolase/ |
| | | | | | | | | | | | | | | enoyl-Coenzyme A | enoyl-Coenzyme A |
| | | | | | | | | | | | | | | hydratase (trifunctional | hydratase (trifunctional |
| | | | | | | | | | | | | | | protein), beta subunit | protein), beta subunit |
| PCSK4 | HG4297-HT4 | HG4297-HT | PASS | 9 | 24.00 | PASS | 13 | 9 | 19.77 | 1.21 | 1.21 | | | | |
| ATM | U33841_at | U33841 | PASS | 5 | 6.20 | PASS | 9 | 5 | 5.11 | 1.21 | 1.21 | ATM | 11q22-q23 | ataxia telangiectasia | ataxia telangiectasia |
| | | | | | | | | | | | | | | mutated (includes com- | mutated (includes com- |
| | | | | | | | | | | | | | | plementation groups A, | plementation groups A, |
| | | | | | | | | | | | | | | C and D) | C and D) |
| RPL31 | X15940_at | X15940 | PASS | 9 | 354.56 | PASS | 13 | 9 | 292.46 | 1.21 | 1.21 | RPL31 | 2 | ribosomal protein L31 | ribosomnal protein L31 |
| BTK_rna3 | U78027_rna3 | U78027 | PASS | 9 | 174.44 | PASS | 13 | 9 | 144.08 | 1.21 | 1.21 | FTP3 | | FTP3 | Lysosomal exoglycosidase |
| X15729_s_at | X15729_s_at | X15729 | PASS | 9 | 36.56 | PASS | 13 | 9 | 30.23 | 1.21 | 1.21 | DDX5 | 17q21 | DEAD/H (Asp-Glu-Ala- | DEAD/H (Asp-Glu-Ala- |
| | | | | | | | | | | | | | | Asp/His) box polypeptide 5 | Asp/His) box polypeptide 5 |
| | | | | | | | | | | | | | | (RNA helicase, 68 kD) | RNA helicase, 68 kD) |
| DNAPKCS | U47077_at | U47077 | PASS | 7 | 5.29 | PASS | 8 | 7 | 4.38 | 1.21 | 1.21 | PRKDC | 8q11 | DNA-dependent protein | protein kinase, DNA- |
| | | | | | | | | | | | | | | kinase catalytic subunit | activated, catalytic poly- |
| | | | | | | | | | | | | | | | peptide |
| CUL3 | U58089_at | U58089 | PASS | 8 | 9.75 | PASS | 13 | 8 | 8.08 | 1.21 | 1.21 | CUL3 | Chr. 2 | cullin 3 | cullin 3 |
| D28473_s_at | D28473_s_at | D28473 | PASS | 6 | 6.33 | PASS | 12 | 6 | 5.25 | 1.21 | 1.21 | IARS | 9q21 | isoleucine-tRNA synthetase | isoleucine-tRNA synthetase |
| J04029_s_at | J04029_s_at | J04029 | PASS | 9 | 9.56 | PASS | 13 | 9 | 7.92 | 1.21 | 1.21 | KRT10 | 17q21-q23 | keratin 10 | keratin 10 (epidermolytic |
| | | | | | | | | | | | | | | | hyperkeratosis; keratosis |
| | | | | | | | | | | | | | | | palmaris et plantaris) |
| CDC16HS | U18291_at | U18291 | PASS | 6 | 6.83 | PASS | 12 | 6 | 5.67 | 1.21 | 1.21 | CDC16 | | cell vidision cycle 16; | cell division cycle 16; |
| | | | | | | | | | | | | | | anaphase promoting | anaphase promoting |
| | | | | | | | | | | | | | | complex 6 | complex 6 |
| HSPE1 | U07550_at | U07550 | PASS | 7 | 7.43 | PASS | 11 | 7 | 6.18 | 1.20 | 1.20 | HSPE1 | | heat shock 10 kD protein 1 | heat shock 10 kD protein 1 |
| | | | | | | | | | | | | | | (chaperonin 10) | chaperonin 10) |
| BTF3_f | HG1515-HT1 | HG1515-HT | PASS | 9 | 63.44 | PASS | 13 | 9 | 52.85 | 1.20 | 1.20 | | | | |
| CD86 | U04343_at | U04343 | PASS | 8 | 6.00 | PASS | 9 | 8 | 5.00 | 1.20 | 1.20 | CD86 | | CD86 antigen | common synonyms are |
| | | | | | | | | | | | | | | | B7-2 and B70; B70 antigen; |
| | | | | | | | | | | | | | | | B7-2 |
| K11 | D13636_at | D13636 | PASS | 6 | 7.50 | PASS | 8 | 6 | 6.25 | 1.20 | 1.20 | GTF3C2 | 2 | general transcription | general transcription |
| | | | | | | | | | | | | | | factor IIIC, polypeptide 2 | factor IIIC, polypeptide 2 |
| | | | | | | | | | | | | | | (beta subunit, 110 kD) | (beta subunit, 110 kD) |
| SEMAE | AB000220_at | AB000220 | PASS | 5 | 6.00 | PASS | 8 | 5 | 5.00 | 1.20 | 1.20 | | | semaphorin E | |
| TRANSP1 | D87127_at | D87127 | PASS | 5 | 6.60 | PASS | 8 | 5 | 5.50 | 1.20 | 1.20 | TLOC1 | | translocation protein 1 | translocation protein 1 |
| X75755_rna1 | X75755_rna1 | X75755 | PASS | 7 | 6.43 | PASS | 11 | 7 | 5.36 | 1.20 | 1.20 | SFRS2 | 17 | splicing factor, arginine/ | splicing factor, arginine/ |
| | | | | | | | | | | | | | | serine-rich 2 | serine-rich 2 |
| PTPN4 | M68941_at | M68941 | PASS | 7 | 7.86 | PASS | 9 | 7 | 6.56 | 1.20 | 1.20 | PTPN4 | | protein tyrosine | protein tyrosine |
| | | | | | | | | | | | | | | phosphatase, non-receptor | phosphatase, non-receptor |
| | | | | | | | | | | | | | | type 4 (megakaryocyte) | type 4 (megakaryocyte) |
| TAF2G | U21858_at | U21858 | PASS | 8 | 7.00 | PASS | 13 | 8 | 5.85 | 1.20 | 1.20 | TAF2G | | TATA box binding protein | TATA box binding protein |
| | | | | | | | | | | | | | | (TBP)-associated factor, | (TBP)-associated factor, |
| | | | | | | | | | | | | | | RNA polymerase II, G, | RNA polymerase II, G, |
| | | | | | | | | | | | | | | 32 kD | 32 kD |
| PIGB | D42138_at | D42138 | PASS | 6 | 5.83 | PASS | 8 | 6 | 4.88 | 1.20 | 1.20 | PIGB | 15q21-q22 | phosphatidylinositol | phosphatidylinositiol |
| | | | | | | | | | | | | | | glycan, class B | glycan, class B |
| U50527_s_at | U50527_s_at | U50527 | PASS | 6 | 5.67 | PASS | 8 | 6 | 4.75 | 1.19 | 1.19 | | | | |
| PYGL | M14636_at | M14636 | PASS | 7 | 5.71 | PASS | 10 | 7 | 4.80 | 1.19 | 1.19 | PYGL | 14q11.2-q24.3 | phosphorylase, glycogen; | phosphorylase, glycogen; |
| | | | | | | | | | | | | | | liver (Hers disease, | liver (Hers disease, |
| | | | | | | | | | | | | | | glycogen storage disease | glycogen storage disease |
| | | | | | | | | | | | | | | type VI) | type VI) |
| PSMZ | D38048_at | D38048 | PASS | 9 | 10.89 | PASS | 13 | 9 | 9.15 | 1.19 | 1.19 | PSMB7 | 9q34.11- | proteasome (prosome, | proteasome (prosome, |
| | | | | | | | | | | | | | q34.12 | macropain) subunit, | macropain) subunit, |
| | | | | | | | | | | | | | | beta type, 7 | beta type, 7 |
| CASP4 | U28014_at | U28014 | PASS | 9 | 23.33 | PASS | 13 | 9 | 19.62 | 1.19 | 1.19 | CASP4 | 11q22.2-q22.3 | caspase 4, apoptosis- | caspase 4, apoptosis- |
| | | | | | | | | | | | | | | related cysteine protease | related cysteine protease |
| IGFBP7 | HG987-HT98 | HG987-HT | PASS | 7 | 8.14 | PASS | 13 | 7 | 6.85 | 1.19 | 1.19 | | | | |
| P23 | L24804_at | l24804 | PASS | 5 | 11.00 | PASS | 12 | 5 | 9.25 | 1.19 | 1.19 | p23 | | | |
| PDHB | D90086_at | D90086 | PASS | 9 | 16.56 | PASS | 13 | 9 | 13.92 | 1.19 | 1.19 | PDHB | 3p21.1-p14.2 | pyruvate dehydrogenase | pyruvate dehydrogenase |
| | | | | | | | | | | | | | | (lipoamide) beta | (lipoamide) beta |
| ENTDFPF | U62136_at | U62136 | PASS | 6 | 7.00 | PASS | 9 | 6 | 5.89 | 1.19 | 1.19 | | | enterocyte differentiation | EDPF-1; putative |
| | | | | | | | | | | | | | | associated factor EDAF-1 | enterocyte differentiation |
| | | | | | | | | | | | | | | | promoting factor |
| TRGGP230 | U41740_at | U41740 | PASS | 6 | 6.33 | PASS | 9 | 6 | 5.33 | 1.19 | 1.19 | GOLGA4 | 6p22-p12 | golgi autoantigen, golgin | golgi autoantigen, golgin |
| | | | | | | | | | | | | | | subfamily a, 4 | subfamily a, 4 |
| PDE4B | L20971_at | L20971 | PASS | 7 | 6.29 | PASS | 10 | 7 | 5.30 | 1.19 | 1.19 | PDE4B | 1p31 | phosphodiesterase 4B, | phosphodiesterase 4B, |
| | | | | | | | | | | | | | | cAMP-specific (dunce | cAMP-specific (dunce |
| | | | | | | | | | | | | | | (Drosophila)-homolog | (Drosophila)-homolog |
| | | | | | | | | | | | | | | phosphodiesterase | phosphodiesterase |
| MLH1 | U07418_at | U07418 | PASS | 9 | 7.11 | PASS | 12 | 9 | 6.00 | 1.19 | 1.19 | hmlh1 | | | human homolog of E. coli |
| | | | | | | | | | | | | | | | mutL gene product, Swiss- |
| | | | | | | | | | | | | | | | Prot Accession Number |
| | | | | | | | | | | | | | | | P23367 |
| CTNNA1 | U03100_at | U03100 | PASS | 5 | 6.40 | PASS | 10 | 5 | 5.40 | 1.19 | 1.19 | CTNNA1 | 5q31 | catenin (cadherin-associated | catenin (cadherin-associated |
| | | | | | | | | | | | | | | protein), alpha 1 (102 kD) | protein), alpha 1 (102 kD) |
| BAP | U72511_at | U72511 | PASS | 9 | 29.78 | PASS | 13 | 9 | 25.15 | 1.18 | 1.18 | hBAP | | B-cell receptor associated | BAP; prohibitin related |
| | | | | | | | | | | | | | | protein | protein, similar to EST with |
| | | | | | | | | | | | | | | | GenBank Accession |
| | | | | | | | | | | | | | | | Number H45225; see |
| | | | | | | | | | | | | | | | corresponding genomic |
| | | | | | | | | | | | | | | | sequence in GenBank |
| | | | | | | | | | | | | | | | Accession Number U72506 |
| PSMA2 | M64992_at | M64992 | PASS | 9 | 14.11 | PASS | 13 | 9 | 11.92 | 1.18 | 1.18 | PSMA1 | 11p15.1 | proteasome (prosome, | proteasome (prosome, |
| | | | | | | | | | | | | | | macropain) subunit, alpha | macropain) subunit, alpha |
| | | | | | | | | | | | | | | type, 1 | type, 1 |
| HINT | U51004_at | U51004 | PASS | 9 | 49.89 | PASS | 13 | 9 | 42.15 | 1.18 | 1.18 | PKCI-1 | | protein kinase C inhibitor | putative protein kinase C |
| | | | | | | | | | | | | | | | inhibitor |
| MPP11 | X98260_at | X98260 | PASS | 5 | 7.00 | PASS | 12 | 5 | 5.92 | 1.18 | 1.18 | mpp11 | | M-phase phosphoprotein 11 | |
| PAM | M37721_at | M37721 | PASS | 9 | 5.56 | PASS | 10 | 9 | 4.70 | 1.18 | 1.18 | PAM | 5q | peptidylglycine alpha- | peptidylglycine alpha- |
| | | | | | | | | | | | | | | amidating monooxygenase | amidating monoxygenase |
| K143_COSC | D63477_at | D63477 | PASS | 8 | 6.00 | PASS | 13 | 8 | 5.08 | 1.18 | 1.18 | KIAA0143 | | | The KIAA0143 gene |
| | | | | | | | | | | | | | | | product is related to a |
| | | | | | | | | | | | | | | | putative C. elegans gene |
| | | | | | | | | | | | | | | | encoded on cosmid C32D5 |
| BRG1 | U29175_at | U29175 | PASS | 9 | 7.78 | PASS | 12 | 9 | 6.58 | 1.18 | 1.18 | BRG1 | | transcriptional activator | similar to product encoded |
| | | | | | | | | | | | | | | | by GenBank Accession |
| | | | | | | | | | | | | | | | Number D26156 |
| H2B_f | M60750_f_at | M60750 | PASS | 7 | 10.71 | PASS | 13 | 7 | 9.08 | 1.18 | 1.18 | H2BFL | 6p21.3 | H2B histone family, | H2B histone family, |
| | | | | | | | | | | | | | | member L | member L |
| SAS | U01160_at | U01160 | PASS | 9 | 5.56 | PASS | 7 | 9 | 4.71 | 1.18 | 1.18 | SAS | | SAS | |
| TP53BP2 | U50078_at | U50078 | PASS | 7 | 8.14 | PASS | 13 | 7 | 6.92 | 1.18 | 1.18 | HERC1 | 15qter-pter | hect (homologous to the | hect (homologous to the |
| | | | | | | | | | | | | | | A6-AP (UBE3A) carboxyl | E6-AP (UBE3A) carboxyl |
| | | | | | | | | | | | | | | terminus) domain and | terminus) domain and |
| | | | | | | | | | | | | | | RCC1 (CHC1)-like domain | RCC1 (CHC1)-like domain |
| | | | | | | | | | | | | | | (RLD) 1 | (RLD) 1 |
| CRE5 | AFFX-CreX- | AFFX-CreX | PASS | 9 | 199.44 | PASS | 13 | 9 | 170.00 | 1.17 | 1.17 | | | | |
| GRL | M10901_at | M10901 | PASS | 8 | 7.13 | PASS | 13 | 8 | 6.08 | 1.17 | 1.17 | GRL | 5q31-q32 | glucocorticoid receptor | glucocorticoid receptor |
| PGE2R | U19487_at | U19487 | PASS | 5 | 5.60 | PASS | 9 | 5 | 4.78 | 1.17 | 1.17 | PTGER2 | 5p13.1 | prostaglandin E receptor 2 | prostaglandin E receptor 2 |
| | | | | | | | | | | | | | | (subtype EP2), 53 kD | (subtype EP2), 53 kD |
| DNAJ | L08069_at | L08069 | PASS | 8 | 11.63 | PASS | 13 | 8 | 9.92 | 1.17 | 1.17 | HSJ2 | | heat shock protein, DNAJ- | heat shock protein, DNAJ- |
| | | | | | | | | | | | | | | like 2 | like 2 |
| HG2815-HT2 | HG2815-HT2 | HG2815-HT | PASS | 9 | 37.67 | PASS | 13 | 9 | 32.15 | 1.17 | 1.17 | | | | |
| GAPDH5 | AFFX-HUM | AFFX-HUM | PASS | 9 | 124.78 | PASS | 13 | 9 | 1.17 | 1.17 | | | | | |
| U30827_s_at | U30827_s_at | U30827 | PASS | 9 | 21.33 | PASS | 13 | 9 | 18.23 | 1.17 | 1.17 | SRp40 | | SRp40-3 | member of the family of SR |
| | | | | | | | | | | | | | | | protein pre-mRNA splicing |
| | | | | | | | | | | | | | | | factors; alternatively spliced |
| ERP31 | X94910_at | X94910 | PASS | 8 | 19.25 | PASS | 13 | 8 | 16.46 | 1.17 | 1.17 | | | ERp28 | Similar to ULA 5 product, |
| | | | | | | | | | | | | | | | AC P30040 |
| TCP1 | X52882_at | X52882 | PASS | 8 | 11.38 | PASS | 12 | 8 | 9.75 | 1.17 | 1.17 | | | | t-complex polypeptide 1 |
| | | | | | | | | | | | | | | | (AA 1-556) |
| DAP5 | AFFX-DapX- | AFFX-Dap | PASS | 9 | 242.44 | PASS | 13 | 9 | 207.85 | 1.17 | 1.17 | | | | |
| SGTPBP | U57094_at | U57094 | PASS | 6 | 11.00 | PASS | 9 | 6 | 9.44 | 1.16 | 1.16 | RAB27A | | RAB27A, member RAS | RAB27A, member RAS |
| | | | | | | | | | | | | | | oncogene family | oncogene family |
| SF2P32 | M69039_at | M69039 | PASS | 8 | 9.13 | PASS | 13 | 8 | 7.85 | 1.16 | 1.16 | CIQBP | 17p13.3 | complement component 1, | complement component 1, |
| | | | | | | | | | | | | | | q subcomponent binding | q subcomponent binding |
| | | | | | | | | | | | | | | protein | protein |
| UBE2A | M74524_at | M74524 | PASS | 7 | 6.00 | PASS | 12 | 7 | 5.17 | 1.16 | 1.16 | UBE2A | Xq24-q25 | ubiquitin-conjugating | ubiquitin-conjugating |
| | | | | | | | | | | | | | | enzyme E2A (RAD6 | enzyme E2A (RAD6 |
| | | | | | | | | | | | | | | homolog) | homolog) |
| CCT4 | U38846_at | U38846 | PASS | 9 | 30.67 | PASS | 13 | 9 | 26.46 | 1.16 | 1.16 | SRB | | stimulator of TAR RNA | |
| | | | | | | | | | | | | | | binding | |
| FRG1 | L76159_at | L76159 | PASS | 6 | 7.67 | PASS | 13 | 6 | 6.62 | 1.16 | 1.16 | FRG1 | 4q35 | FSHD region gene 1 | FSHD region gene 1 |
| M57466_s_at | M57466_s_at | M57466 | PASS | 9 | 70.67 | PASS | 13 | 9 | 61.08 | 1.16 | 1.16 | HLA-DPB1 | 6p21.3 | major histocompatibility | major histocompatibility |
| | | | | | | | | | | | | | | complex, class II, DP | complex, class II, DP |
| | | | | | | | | | | | | | | beta 1 | beta 1 |
| U44799_s_at | U44799_s_at | U44799 | PASS | 6 | 6.00 | PASS | 10 | 6 | 5.20 | 1.15 | 1.15 | | | U1-snRNP binding protein | |
| | | | | | | | | | | | | | | homolog | |
| RASA1 | M23379_at | M23379 | PASS | 5 | 6.00 | PASS | 10 | 5 | 5.20 | 1.15 | 1.15 | RASA1 | 5q13 | RAS p21 protein activator | RAS p21 protein activator |
| | | | | | | | | | | | | | | (GTPase activating protein) | (GTPase activating protein) |
| | | | | | | | | | | | | | | 1 | 1 |
| J0621_s_at | J02621_s_at | J02621 | PASS | 9 | 26.44 | PASS | 13 | 9 | 22.92 | 1.15 | 1.15 | HMG14 | 21q22.3 | | high-mobility group |
| | | | | | | | | | | | | | | | (nonhistone chromosomal) |
| | | | | | | | | | | | | | | | protein 14 |
| YES1 | M15990_at | M15990 | PASS | 7 | 5.43 | PASS | 7 | 7 | 4.71 | 1.15 | 1.15 | YES1 | 18p11.31- | | v-yes-1 Yamaguchi |
| | | | | | | | | | | | | | p11.21 | | sarcoma viral oncogene |
| | | | | | | | | | | | | | | | homolog 1 |
| CTSL | X12451_at | X12451 | PASS | 6 | 6.33 | PASS | 10 | 6 | 5.50 | 1.15 | 1.15 | CTSL | 9q21-q22 | cathepsin L | cathepsin L |
| WI9119 | M24069_at | M24069 | PASS | 7 | 6.71 | PASS | 12 | 7 | 5.83 | 1.15 | 1.15 | CSDA | 12p13.1 | cold shock domain protein | cold shock domain protein |
| | | | | | | | | | | | | | | A | A |
| Z25521_s_at | Z25521_s_at | Z25521 | PASS | 9 | 16.11 | PASS | 13 | 9 | 14.00 | 1.15 | 1.15 | | | integrin associated protein | |
| CISACTS | M82882_at | M82882 | PASS | 7 | 7.86 | PASS | 12 | 7 | 6.83 | 1.15 | 1.15 | ELF1 | | | E74-like factor 1 (ets |
| | | | | | | | | | | | | | | | domain transcription factor) |
| K181 | D80003_at | D80003 | PASS | 8 | 6.13 | PASS | 12 | 8 | 5.33 | 1.15 | 1.15 | KIAA0181 | | | |
| CAMLG | U18242_at | U18242 | PASS | 7 | 6.57 | PASS | 11 | 7 | 5.73 | 1.15 | 1.15 | CAMLG | 5q23 | calcium modulating ligand | calcium modulating ligand |
| RANBP1 | D38076_at | D38076 | PASS | 7 | 9.00 | PASS | 13 | 7 | 7.85 | 1.15 | 1.15 | RANBP1 | | RAN binding protein 1 | RAN binding protein 1 |
| ICT1 | X81788_at | X81788 | PASS | 7 | 6.14 | PASS | 11 | 7 | 5.36 | 1.15 | 1.15 | ICT1 | | immature colon carcinoma | immature colon carcinoma |
| | | | | | | | | | | | | | | transcript 1 | transcript 1 |
| RB1 | L41870_at | L41870 | PASS | 7 | 5.86 | PASS | 8 | 7 | 5.13 | 1.14 | 1.14 | RB1 | 13q14.2 | retinoblastoma 1 (including | retinoblastoma 1 (including |
| | | | | | | | | | | | | | | osteosarcoma) | osteosarcoma) |
| K254 | D87443_at | D87443 | PASS | 7 | 4.57 | PASS | 7 | 7 | 4.00 | 1.14 | 1.14 | KIAA0254 | | | KIAA0254 gene product |
| HG1428-HT1 | HG1428-HT1 | HG1428-HT | PASS | 9 | 286.67 | PASS | 13 | 9 | 250.92 | 1.14 | 1.14 | | | | |
| M31520_rna1 | M31520_rna1 | M31520 | PASS | 9 | 154.78 | PASS | 13 | 9 | 135.54 | 1.14 | 1.14 | rps24 | | ribosomal protein S24 | unknown protein |
| NAP1L1 | M86667_at | M86667 | PASS | 9 | 33.78 | PASS | 13 | 9 | 29.62 | 1.14 | 1.14 | NAP1L1 | | nucleosome assembly | nucleosome assembly |
| | | | | | | | | | | | | | | protein 1-like 1 | protein 1-like 1 |
| RBM3 | U28686_at | U28686 | PASS | 8 | 12.13 | PASS | 11 | 8 | 10.64 | 1.14 | 1.14 | RBM3 | Xp11.2 | RNPL | RNA binding motif protein |
| | | | | | | | | | | | | | | | 3 |
| U58046_s_at | U58046_s_at | U58046 | PASS | 5 | 5.80 | PASS | 10 | 5 | 5.10 | 1.14 | 1.14 | EIFS310 | 10q26 | eukaryotic translation | eukaryotic translation |
| | | | | | | | | | | | | | | initiation factor 3, subunit | initiation factor 3, subunit |
| | | | | | | | | | | | | | | 10, (theta, 170 kD) | 10, (theta, 170 kD) |
| VBP1 | U56833_at | U56833 | PASS | 8 | 6.38 | PASS | 13 | 8 | 5.62 | 1.14 | 1.14 | VBP1 | Xq28 | von Hippel-Lindau | von Hippel-Lindau |
| | | | | | | | | | | | | | | binding protein 1 | binding protein 1 |
| TCLLYM_rna | X82240_rna1 | X82240 | PASS | 5 | 15.20 | PASS | 12 | 5 | 13.42 | 1.13 | 1.13 | TCL1A | 14q32.1 | T cell leukemia/lymphoma | T cell leukemia/lymphoma |
| | | | | | | | | | | | | | | 1 | 1 |
| ACP1 | U25849_at | bU25849 | PASS | 6 | 9.83 | PASS | 13 | 6 | 8.69 | 1.13 | 1.13 | ACP1 | 2p25 | acid phosphatase 1, | acid phosphatase 1, |
| | | | | | | | | | | | | | | soluble | soluble |
| M60483_rna | M60483_rna | M60483 | PASS | 8 | 6.50 | PASS | 12 | 8 | 5.75 | 1.13 | 1.13 | PPP2CA | 5q23-q31 | protein phosphatase 2 | protein phosphatase 2 |
| | | | | | | | | | | | | | | (formerly 2A), catalytic | (formerly 2A), catalytic |
| | | | | | | | | | | | | | | subunit, alpha isoform | subunit, alpha isoform |
| PSMHC8 | D00762_at | D00762 | PASS | 7 | 11.43 | PASS | 9 | 7 | 10.11 | 1.13 | 1.13 | PSMA3 | 14q23 | proteasome (prosome, | proteasome (prosome, |
| | | | | | | | | | | | | | | macropain) subunit, alpha | macropain) subunit, alpha |
| | | | | | | | | | | | | | | type, 3 | type, 3 |
| LGALS2 | M87860_at | M87860 | PASS | 7 | 26.71 | PASS | 11 | 7 | 23.64 | 1.13 | 1.13 | LGALS2 | | S-lac lectin | |
| D11327_s_at | D11327_s_at | D11327 | PASS | 8 | 5.75 | PASS | 10 | 8 | 5.10 | 1.13 | 1.13 | PTPN7 | 1q32.1 | protein tyrosine | protein tyrosine |
| | | | | | | | | | | | | | | phosphatase, non-receptor | phosphatase, non-receptor |
| | | | | | | | | | | | | | | type 7 | type 7 |
| EPS3 | L34587_at | L34587 | PASS | 9 | 13.78 | PASS | 13 | 9 | 12.23 | 1.13 | 1.13 | | | RNA polymerase II elonga- | putative |
| | | | | | | | | | | | | | | tion factor SIII, p15 subunit | |
| FEZ2 | U60061_at | U60061 | PASS | 6 | 5.83 | PASS | 11 | 6 | 5.18 | 1.13 | 1.13 | FEZ2 | | FEZ2 | zygin 2 |
| U49020_cds2 | U49020_cds2 | U49020 | PASS | 8 | 5.75 | PASS | 9 | 8 | 5.11 | 1.13 | 1.13 | MEF2A | | myocyte-specific enhancer | |
| | | | | | | | | | | | | | | factor 2A, C4 form | |
| PPM1A | S87759_at | S87759 | PASS | 7 | 6.00 | PASS | 9 | 7 | 5.33 | 1.13 | 1.13 | PPM1B | | protein phosphatase 1B | protein phosphatase 1B |
| | | | | | | | | | | | | | | (formerly 2C), magnesium- | (formerly 2C), magnesium- |
| | | | | | | | | | | | | | | dependent, beta isoform | dependent, beta isoform |
| EIF4A2 | D30655_at | D30655 | PASS | 9 | 60.56 | PASS | 13 | 9 | 53.85 | 1.12 | 1.12 | EIF4A2 | 18p11.2 | eukaryotic translation | eukaryotic translation |
| | | | | | | | | | | | | | | initiation factor 4A, | initiation factor 4A, |
| | | | | | | | | | | | | | | isoform 2 | isoform 2 |
| GRLB | HG4582-HT4 | HG4582-HT | PASS | 5 | 5.60 | PASS | 11 | 5 | 5.00 | 1.12 | 1.12 | | | | |
| U12387_s_at | U12387_s_at | U12387 | PASS | 5 | 6.60 | PASS | 10 | 5 | 5.90 | 1.12 | 1.12 | TPMT | | thiopurine methyl- | 35 kDa monomer, cytosolic |
| | | | | | | | | | | | | | | transferase | protein |
| AICL | X96719_at | X96719 | PASS | 5 | 18.40 | PASS | 13 | 5 | 16.46 | 1.12 | 1.12 | CLECSF2 | 12p | C-type lectin | C-type (calcium dependent, |
| | | | | | | | | | | | | | | | carbohydrate-recognition |
| | | | | | | | | | | | | | | | domain) lectin, superfamily |
| | | | | | | | | | | | | | | | member 2 (activation- |
| | | | | | | | | | | | | | | | induced) |
| K69_DS0556 | D31885_at | D31885 | PASS | 9 | 16.56 | PASS | 13 | 9 | 14.85 | 1.12 | 1.12 | KIAA0069 | | | |
| PSMA5 | X61970_at | X61970 | PASS | 9 | 13.11 | PASS | 13 | 9 | 11.77 | 1.11 | 1.11 | PSMA5 | 1p13 | proteasome (prosome, | proteasome (prosome, |
| | | | | | | | | | | | | | | macropain) subunit, alpha | macropain) subunit, alpha |
| | | | | | | | | | | | | | | type, 5 | type, 5 |
| PRPS1 | D00860_at | D00860 | PASS | 9 | 6.11 | PASS | 12 | 9 | 5.50 | 1.11 | 1.11 | PRPS1 | Xq21-q27 | phosphoribosyl pyro- | phosphoribosyl pyro- |
| | | | | | | | | | | | | | | phosphate synthetase 1 | phosphate synthetase 1 |
| U33936_s_at | U33936_s_at | U33936 | PASS | 7 | 5.86 | PASS | 11 | 7 | 5.27 | 1.11 | 1.11 | ADK | 10cen-q24 | adenosine kinase | adenosine kinase |
| LCP2 | U20158_at | U20158 | PASS | 9 | 17.00 | PASS | 13 | 9 | 15.31 | 1.11 | 1.11 | | | SLP-76 | 76 kDa tyrosine phospho- |
| | | | | | | | | | | | | | | | protein |
| HG2981-HT3 | HG2981-HT3 | HG2981-HT | PASS | 7 | 10.29 | PASS | 11 | 7 | 9.27 | 1.11 | 1.11 | | | | |
| PPIB | M63573_at | M63573 | PASS | 7 | 17.29 | PASS | 12 | 7 | 15.58 | 1.11 | 1.11 | PPIB | 15 | peptidylprolyl isomerase B | peptidylprolyl isomerase B |
| | | | | | | | | | | | | | | (cyclophilin B) | (cyclophilin B) |
| B56E | L76703_at | L76703 | PASS | 6 | 5.17 | PASS | 9 | 6 | 4.67 | 1.11 | 1.11 | PPP2R5E | 7p11.2-p12 | protein phosphatase B56- | protein phosphatase 2, |
| | | | | | | | | | | | | | | epsilon | regulatory subunit B (B56), |
| | | | | | | | | | | | | | | | epsilon isoform |
| E_23801 | U79282_at | U79282 | PASS | 5 | 5.20 | PASS | 10 | 5 | 4.70 | 1.11 | 1.11 | | | | |
| HSPA8 | HG2855-HT2 | HG2855-HT | PASS | 9 | 38.56 | PASS | 13 | 9 | 35.00 | 1.10 | 1.10 | | | | |
| H2AZ | M37583_at | M37583 | PASS | 9 | 18.11 | PASS | 13 | 9 | 16.46 | 1.10 | 1.10 | H2AFZ | 4q24 | H2A histone family, | H2A histone family, |
| | | | | | | | | | | | | | | member Z | member Z |
| PHCII | J04973_at | J04973 | PASS | 9 | 12.78 | PASS | 13 | 9 | 11.62 | 1.10 | 1.10 | UQCRC2 | 16p12 | ubiquinol-cytochrome c | ubiquinol-cytochrome c |
| | | | | | | | | | | | | | | reductase core protein II | reductase core protein II |
| TCRD | M21624_at | M21624 | PASS | 9 | 17.33 | PASS | 13 | 9 | 15.77 | 1.10 | 1.10 | TCRD | 14q11.2 | T-cell receptor, delta | T-cell receptor, delta |
| | | | | | | | | | | | | | | (V, D, J, C) | (V, D, J, C) |
| TNFAIP3 | M59465_at | M59465 | PASS | 9 | 13.33 | PASS | 13 | 9 | 12.15 | 1.10 | 1.10 | TNFAIP1 | 17q22-q23 | A20 | tumor necrosis factor, |
| | | | | | | | | | | | | | | | alpha-induced protein 1 |
| | | | | | | | | | | | | | | | (endothelial) |
| APBB1 | U50939_at | U50939 | PASS | 7 | 6.57 | PASS | 12 | 7 | 6.00 | 1.10 | 1.10 | APPBP1 | 16q22 | Amyloid beta precursor | amyloid beta precursor |
| | | | | | | | | | | | | | | protein binding protein 1 | protein-binding protein 1, |
| | | | | | | | | | | | | | | | 59 kD |
| BACTINM | AFFX-HSAC | AFFX-HSA | PASS | 9 | 330.67 | PASS | 13 | 9 | 302.92 | 1.09 | 1.09 | | | | |
| D13S824E | U47635_at | U47635 | PASS | 6 | 6.67 | PASS | 9 | 6 | 6.11 | 1.09 | 1.09 | | | | |
| RAD23B | D21090_at | D21090 | PASS | 7 | 6.00 | PASS | 12 | 7 | 5.50 | 1.09 | 1.09 | RAD23B | 3p25.1 | RAD23 (S. cerevisiae) | RAD23 (S. cerevisiae) |
| | | | | | | | | | | | | | | homolog B | homolog B |
| PSMD4 | U24704_at | U24704 | PASS | 7 | 11.29 | PASS | 11 | 7 | 10.36 | 1.09 | 1.09 | | | antisecretory factor-1 | similar to Human S5a |
| | | | | | | | | | | | | | | | protosome subunit, |
| | | | | | | | | | | | | | | | GenBank Accession |
| | | | | | | | | | | | | | | | Number U51007 |
| PRKMK1 | L11284_at | L11284 | PASS | 7 | 10.29 | PASS | 13 | 7 | 9.46 | 1.09 | 1.09 | PRKMK1 | 15q22.1- | | protein kinase, mitogen- |
| | | | | | | | | | | | | | q22.33 | | activated, kinase 1 (MAP) |
| | | | | | | | | | | | | | | | kinase kinase 1) |
| BIOB5 | AFFX-BioB- | AFFX-BioB | PASS | 9 | 113.56 | PASS | 13 | 9 | 104.69 | 1.08 | 1.08 | | | | |
| LKP | L13848_at | L13848 | PASS | 6 | 10.83 | PASS | 10 | 6 | 10.00 | 1.08 | 1.08 | DDX9 | 1q25 | DEAD/H (Asp-Glu-Ala- | DEAD/H (Asp-Glu-Ala- |
| | | | | | | | | | | | | | | Asp/His) box polypeptide 9 | Asp/His) box polypeptide 9 |
| | | | | | | | | | | | | | | (RNA helicase A, nuclear | (RNA helicase A, nuclear |
| | | | | | | | | | | | | | | DNA helicase II) | DNA helicase II); |
| | | | | | | | | | | | | | | | leukophysin) |
| EFTS | L37936_at | L37936 | PASS | 8 | 10.38 | PASS | 12 | 8 | 9.58 | 1.08 | 1.08 | | | elongation factor Ts | |
| CBX | U35451_at | U35451 | PASS | 6 | 6.17 | PASS | 10 | 6 | 5.70 | 1.08 | 1.08 | p25beta | | heterochromatin protein | |
| | | | | | | | | | | | | | | | p25 |
| SMN1_rna3 | U80017_rna3 | U80017 | PASS | 7 | 6.00 | PASS | 9 | 7 | 5.56 | 1.08 | 1.08 | btf2p44 | | basic transcription factor 2 | NAIP |
| | | | | | | | | | | | | | | p44 | |
| X03689_s_at | X03689_s_at | X03689 | PASS | 9 | 224.56 | PASS | 13 | 9 | 208.08 | 1.08 | 1.08 | EEF1A2 | 20q13.3 | eukaryotic translation | eukaryotic translation |
| | | | | | | | | | | | | | | elongation factor 1 alpha 2 | elongation factor 1 alpha 2 |
| PRPS2 | Y00971_at | Y00971 | PASS | 5 | 5.00 | PASS | 11 | 5 | 4.64 | 1.08 | 1.08 | PRPS2 | Xpter-q21 | phosphoribosyl pyro- | phosphoribosyl pyro- |
| | | | | | | | | | | | | | | phosphate synthetase 2 | phosphate synthetase 2 |
| ATP5C1 | D16562_at | D16562 | PASS | 9 | 29.67 | PASS | 13 | 9 | 27.54 | 1.08 | 1.08 | | | ATP synthase gamma- | L(liver)-type ATP synthase |
| | | | | | | | | | | | | | | subunit | gamma-subunit |
| K191 | D83776_at | D83776 | PASS | 5 | 6.00 | PASS | 7 | 5 | 5.57 | 1.08 | 1.08 | KIAA0191 | | | The KIAA0191 gene is |
| | | | | | | | | | | | | | | | expressed ubiquitously.; |
| | | | | | | | | | | | | | | | The KIAA0191 protein |
| | | | | | | | | | | | | | | | retains the C2H2 zinc- |
| | | | | | | | | | | | | | | | finger at its N-terminal |
| | | | | | | | | | | | | | | | region. |
| M67468_s_at | M67468_s_at | M67468 | PASS | 7 | 5.14 | PASS | 9 | 7 | 4.78 | 1.08 | 1.08 | FMR1 | Xq27.3 | fragile X mental retarda- | fragile X mental retarda- |
| | | | | | | | | | | | | | | tion 1 | tion 1 |
| ANX7 | J04543_at | J04543 | PASS | 8 | 8.25 | PASS | 12 | 8 | 7.67 | 1.08 | 1.08 | ANX7 | 10q21.1-q21.2 | annexin VII | annexin VII (synexin) |
| CMKBR7 | L08177_at | L08177 | PASS | 8 | 5.38 | PASS | 10 | 8 | 5.00 | 1.08 | 1.08 | EB12 | 13 | EB1 2: EBV induced G- | Epstein-Barr virus induced |
| | | | | | | | | | | | | | | protein coupled receptor | gene 2 (lymphocyte- |
| | | | | | | | | | | | | | | | specific G protein-coupled |
| | | | | | | | | | | | | | | | receptor) |
| NIFUL | U47101_at | U47101 | PASS | 9 | 26.78 | PASS | 13 | 9 | 24.92 | 1.07 | 1.07 | hNifU | | NifU-like protein | Similar to N-terminal |
| | | | | | | | | | | | | | | | regions of diazatroph NiFU |
| | | | | | | | | | | | | | | | proteins |
| K242_HYP5 | D87684_at | D87684 | PASS | 8 | 8.13 | PASS | 10 | 8 | 7.60 | 1.07 | 1.07 | KIAA0242 | | | similar to a C. elegans |
| | | | | | | | | | | | | | | | ZK353.8 protein (S44655) |
| LCP1 | J02923_at | J02923 | PASS | 9 | 45.67 | PASS | 13 | 9 | 42.77 | 1.07 | 1.07 | LCP1 | 13q14.3 | lymphocyte cytosolic | lymphocyte cytosolic |
| | | | | | | | | | | | | | | protein 1 (L-plastin) | protein 1 (L-plastin) |
| S203_15 | L40395_at | L40395 | PASS | 8 | 6.50 | PASS | 9 | 8 | 6.11 | 1.06 | 1.06 | | | | ORF; putative |
| LAML | HG1078-HT1 | HG1078-HT | PASS | 7 | 13.00 | PASS | 13 | 7 | 12.23 | 1.06 | 1.06 | | | | |
| PBX3 | X59841_at | X59841 | PASS | 6 | 5.67 | PASS | 9 | 6 | 5.33 | 1.06 | 1.06 | PBX3 | 9q33-q34 | homeobox protein | pre-B-cell leukemia |
| | | | | | | | | | | | | | | | transcription factor 3 |
| SRP9 | U20998_at | U20998 | PASS | 9 | 15.44 | PASS | 13 | 9 | 14.54 | 1.06 | 1.06 | SRP9 | | signal recognition particle | signal recognition particle |
| | | | | | | | | | | | | | | 9 kD | 9 kD |
| L33930_s_at | L33930_s_at | L33930 | PASS | 6 | 4.67 | PASS | 10 | 6 | 4.40 | 1.06 | 1.06 | | | signal transducer CD24 | |
| CGR19 | U66469_at | U66469 | PASS | 5 | 5.40 | PASS | 10 | 5 | 5.10 | 1.06 | 1.06 | | | cell growth regulator | |
| | | | | | | | | | | | | | | CGR19 | |
| SRPK2 | U88666_at | U88666 | PASS | 8 | 5.38 | PASS | 12 | 8 | 5.08 | 1.06 | 1.06 | SRPK2 | 7q22-q31.1 | SFRS protein kinase 2 | SFRS protein kinase 2 |
| B2M | S82297_at | S82297 | PASS | 9 | 164.00 | PASS | 13 | 9 | 155.15 | 1.06 | 1.06 | B2M | 15q21-q22.2 | beta-2-microglobulin | beta-2-microglobulin |
| SRP54 | U51920_at | U51920 | PASS | 6 | 5.17 | PASS | 10 | 6 | 4.90 | 1.05 | 1.05 | SRP54 | | signal recognition particle | signal recognition particle |
| | | | | | | | | | | | | | | | 54 kD |
| U84011_s_at | U84011_s_at | U84011 | PASS | 5 | 4.80 | PASS | 9 | 5 | 4.56 | 1.05 | 1.05 | AGL | 1p21 | amylo-1,6-glucosidase, 4- | amyl-1,6-glucosidase,4- |
| | | | | | | | | | | | | | | alpha-glucanotransferase, | alpha-glucanotransferase, |
| | | | | | | | | | | | | | | isoform 3 | (glycogen debranching |
| | | | | | | | | | | | | | | | enzyme, glycogen storage |
| | | | | | | | | | | | | | | | disease type III) |
| E_23865 | U90912_at | U90912 | PASS | 7 | 5.71 | PASS | 7 | 7 | 5.43 | 1.05 | 1.05 | | | | |
| SFRS3 | U30825_at | U30825 | PASS | 9 | 31.44 | PASS | 13 | 9 | 29.92 | 1.05 | 1.05 | SFRS9 | | splicing factor, arginine/ | splicing factor, arginine/ |
| | | | | | | | | | | | | | | serine-rich 9 | serine-rich 9 |
| HNRPH2 | U01923_at | U01923 | PASS | 7 | 6.71 | PASS | 10 | 7 | 6.40 | 1.05 | 1.05 | | | | |
| RPL14 | D87735_at | D87735 | PASS | 9 | 150.33 | PASS | 13 | 9 | 143.31 | 1.05 | 1.05 | RPL14 | | ribosomal protein L14 | ribosomal protein L14 |
| W52B_f | HG3576-HT3 | HG3576-HT | PASS | 9 | 106.89 | PASS | 13 | 9 | 102.08 | 1.05 | 1.05 | | | | |
| AMFR | M63175_at | M63175 | PASS | 5 | 6.80 | PASS | 8 | 5 | 6.50 | 1.05 | 1.05 | AMFR | 16q21 | autocrine motility factor | autocrine motility factor |
| | | | | | | | | | | | | | | receptor | receptor |
| K73_CYCRP | D38552_at | D38552 | PASS | 6 | 6.83 | PASS | 13 | 6 | 6.54 | 1.05 | 1.05 | KIAA0073 | | | The ha1539 protein is |
| | | | | | | | | | | | | | | | related to cyclophilin |
| MCM3 | D38073_at | D38073 | PASS | 8 | 6.88 | PASS | 10 | 8 | 6.60 | 1.04 | 1.04 | MCM3 | 6p12 | minichromosome main- | minichromosome main- |
| | | | | | | | | | | | | | | tenance deficient (S. | tenance deficient (S. |
| | | | | | | | | | | | | | | cerevisiae) 3 | cerevisiae) 3 |
| PAK3 | U25975_at | U25975 | PASS | 5 | 5.60 | PASS | 10 | 5 | 5.40 | 1.04 | 1.04 | hPAK65 | | hPAK65 | rac/CDC42Hs activated |
| | | | | | | | | | | | | | | | kinase; serine kinase; |
| | | | | | | | | | | | | | | | Method: conceptual trans- |
| | | | | | | | | | | | | | | | lation supplied by author |
| U67122_s_at | U67122_s_at | U67122 | PASS | 7 | 11.00 | PASS | 13 | 7 | 10.62 | 1.04 | 1.04 | | | SUMO-1 | conjugated post-transla- |
| | | | | | | | | | | | | | | | tionally to RanGAP1; |
| | | | | | | | | | | | | | | | ubiquitin-related protein; |
| | | | | | | | | | | | | | | | similar to UBL1 encoded |
| | | | | | | | | | | | | | | | by GenBank Accession |
| | | | | | | | | | | | | | | | Number U38784, PIC1 |
| | | | | | | | | | | | | | | | encoded by GenBank |
| | | | | | | | | | | | | | | | Accession Number U61397 |
| | | | | | | | | | | | | | | | and GMP1 encoded by |
| | | | | | | | | | | | | | | | GenBank Accession |
| | | | | | | | | | | | | | | | Number U72722 |
| L06797_s_at | L06797_s_at | L06797 | PASS | 9 | 33.67 | PASS | 13 | 9 | 32.54 | 1.03 | 1.03 | CXCR4 | 2q21 | Neuropeptide Y receptor | chemokine (C-X-C motif), |
| | | | | | | | | | | | | | | Y3 | receptor 4 (fusin) |
| CAT_rna1 | X04085_rna1 | X04085 | PASS | 7 | 15.43 | PASS | 13 | 7 | 14.92 | 1.03 | 1.03 | | | catalase | |
| SFRS3 | D28423_at | D28423 | PASS | 6 | 17.33 | PASS | 12 | 6 | 16.83 | 1.03 | 1.03 | | | pre-mRNA splicing factor | |
| | | | | | | | | | | | | | | SRp20 | |
| J02683_s_at | J02683_s_at | J02683 | PASS | 9 | 31.56 | PASS | 13 | 9 | 30.69 | 1.03 | 1.03 | ANT2 | Xq24-q26 | adenine nucleotide trans- | adenine nucleotide trans- |
| | | | | | | | | | | | | | | locator 2 (fibroblast) | locator 2 (fibroblast) |
| RPS7_rna1 | Z25749_rna1 | Z25749 | PASS | 9 | 136.56 | PASS | 13 | 9 | 132.85 | 1.03 | 1.03 | RPS7 | 2p25 | ribosomal protein S7 | ribosomal protein S7 |
| HDAC2 | U31814_at | U31814 | PASS | 9 | 6.78 | PASS | 10 | 9 | 6.60 | 1.03 | 1.03 | HDAC2 | 6q21 | histone deacetylase 2 | histone deacetylase 2 |
| M60974_s_at | M60974_s_at | M60974 | PASS | 5 | 5.60 | PASS | 10 | 5 | 5.50 | 1.02 | 1.02 | DDIT1 | 1p34-p12 | DNA-damage-inducible | DNA-damage-inducible |
| | | | | | | | | | | | | | | transcript 1 | transcript 1 |
| SEMA | U60800_at | U60800 | PASS | 9 | 22.22 | PASS | 13 | 9 | 21.85 | 1.02 | 1.02 | CD100 | | semaphorin | |
| D13631_s_at | D13631_s_at | D13631 | PASS | 7 | 11.00 | PASS | 12 | 7 | 10.83 | 1.02 | 1.02 | KIAA0006 | | | |
| PRKACB | M34181_at | M34181 | PASS | 5 | 6.80 | PASS | 10 | 5 | 6.70 | 1.01 | 1.01 | PRKACB | 1 | protein kinase, cAMP- | protein kinase, cAMP- |
| | | | | | | | | | | | | | | dependent, catalytic, beta | dependent, catalytic, beta |
| M26730_s_at | M26730_s_at | M26730 | PASS | 9 | 33.78 | PASS | 13 | 9 | 33.31 | 1.01 | 1.01 | UQBP | | | ubiquinone-binding protein |
| | | | | | | | | | | | | | | | (QP) |
| GD12 | D13988_at | D13988 | PASS | 6 | 18.17 | PASS | 13 | 6 | 18.00 | 1.01 | 1.01 | GD12 | 10p15 | GDP dissociation inhibitor | GDP dissociation inhibitor |
| | | | | | | | | | | | | | | 2 | 2 |
| RPL44 | M15661_at | M15661 | PASS | 9 | 51.44 | PASS | 13 | 9 | 51.08 | 1.01 | 1.01 | RPL44 | | ribosomal protein L44 | ribosomal protein L44 |
| ZFPRES24_2 | AB000468_at | AB000468 | PASS | 8 | 18.75 | PASS | 13 | 8 | 18.69 | 1.00 | 1.00 | RNF4 | 4p16.3 | ring finger protein 4 | ring finger protein 4 |
| BACTIN5 | AFFX-HSAC | AFFX-HSA | PASS | 9 | 240.44 | PASS | 13 | 9 | 240.00 | 1.00 | 1.00 | | | | |
| M87507_s_at | M87507_s_at | M87507 | PASS | 6 | 7.50 | PASS | 12 | 6 | 7.50 | 1.00 | 1.00 | CASP1 | 11q23 | caspase 1, apoptosis-related | caspase 1, apoptosis-related |
| | | | | | | | | | | | | | | cysteine protease | cysteine protease |
| | | | | | | | | | | | | | | (interleukin 1, beta, | (interleukin 1, beta, |
| | | | | | | | | | | | | | | convertase) | convertase) |
| UVRAG_rna1 | X99050_rna1 | X99050 | PASS | 6 | 5.00 | PASS | 11 | 6 | 5.00 | 1.00 | 1.00 | UVRAG | 11q13 | UV radiation resistance | UV radiation resistance |
| | | | | | | | | | | | | | | associated gene | associated gene |
| RP3 | U02556_at | U025556 | PASS | 5 | 6.00 | PASS | 12 | 5 | 6.00 | 1.00 | 1.00 | | | RP3 candidate gene | |
| X58528_s_at | X58528_s_at | X58528 | PASS | 5 | 5.20 | PASS | 10 | 5 | 5.20 | 1.00 | 1.00 | PMP70 | | 70 kDa peroxisomal | |
| | | | | | | | | | | | | | | membrane protein | |
| SFRS3 | L10838_at | L10838 | PASS | 8 | 17.13 | PASS | 13 | 8 | 17.23 | 0.99 | −1.01 | SFRS3 | | splicing factor, arginine/ | splicing factor, arginine/ |
| | | | | | | | | | | | | | | serine-rich 3 | serine-rich 3 |
| METPEP | U29607_at | U29607 | PASS | 9 | 27.22 | PASS | 13 | 9 | 27.69 | 0.98 | −1.02 | | | methionine aminopeptidase | |
| K253 | D87442_at | D87442 | PASS | 6 | 8.17 | PASS | 9 | 6 | 8.33 | 0.98 | −1.02 | KIAA0253 | | | |
| CRE3 | AFFX-CreX- | AFFX-CreX | PASS | 9 | 27.78 | PASS | 13 | 9 | 28.46 | 0.98 | −1.02 | | | | |
| UBE2D3 | U39318_at | U39318 | PASS | 8 | 19.00 | PASS | 13 | 8 | 19.54 | 0.97 | −1.03 | UBCH5C | | UbcH5C | Transcript is widely |
| | | | | | | | | | | | | | | | expressed. Related to S. |
| | | | | | | | | | | | | | | | Cerevisiae UBC4 and |
| | | | | | | | | | | | | | | | UBC5. Closely related to |
| | | | | | | | | | | | | | | | human UbcH5(A) and to |
| | | | | | | | | | | | | | | | UbcH5B |
| U26312_s_at | U26312_s_at | U26312 | PASS | 6 | 5.67 | PASS | 12 | 6 | 5.83 | 0.97 | −1.03 | | | HP1Hs gamma | similar to Drosphila |
| | | | | | | | | | | | | | | | heterochromatin protein |
| | | | | | | | | | | | | | | | HP1 Swiss-Prot Accession |
| | | | | | | | | | | | | | | | Accession Number P29227, |
| | | | | | | | | | | | | | | | and to human hetero- |
| | | | | | | | | | | | | | | | chromatin protein HP1Hs- |
| | | | | | | | | | | | | | | | alpha encoded by GenBank |
| | | | | | | | | | | | | | | | Accession Number |
| | | | | | | | | | | | | | | | U26311; contains chromo |
| | | | | | | | | | | | | | | | domain; recognized by |
| | | | | | | | | | | | | | | | autoantibodies from some |
| | | | | | | | | | | | | | | | patients with scleroderma; |
| | | | | | | | | | | | | | | | heterochromatin protein |
| H33B | Z48950_at | Z48950 | PASS | 9 | 66.78 | PASS | 13 | 9 | 68.85 | 0.97 | −1.03 | H3F3B | 17q25 | histone H3.3 | H3 histone, family 3B |
| | | | | | | | | | | | | | | | (H3.3B) |
| NHRPF | L28010_at | L28010 | PASS | 9 | 14.89 | PASS | 13 | 9 | 15.38 | 0.97 | −1.03 | NHRPF | 10q11.21 | HuRNP F protein | heterogeneous nuclear |
| | | | | | | | | | | | | | | | ribonucleoprotein F |
| E_23665 | U90913_at | U90913 | PASS | 7 | 6.00 | PASS | 10 | 7 | 6.20 | 0.97 | −1.03 | | | | |
| YB1 | J03827_at | J03827 | PASS | 9 | 70.00 | PASS | 13 | 9 | 72.38 | 0.97 | −1.03 | YB1 | 1p34 | | Major histocompatibility |
| | | | | | | | | | | | | | | | complex, class II, Y box- |
| | | | | | | | | | | | | | | | binding protein I; DNA- |
| | | | | | | | | | | | | | | | binding protein B |
| ID2B | M96843_at | M96843 | PASS | 9 | 7.33 | PASS | 13 | 9 | 7.62 | 0.96 | −1.04 | Id2B | | contractile protein | |
| MGC24 | D14043_at | D14043 | PASS | 9 | 14.22 | PASS | 13 | 9 | 14.77 | 0.96 | −1.04 | | | MGC-24 precursor | |
| NDUFV3 | X99728_at | X99728 | PASS | 8 | 8.63 | PASS | 13 | 8 | 9.00 | 0.96 | −1.04 | | | | |
| MTND1 | L04490_at | L04490 | PASS | 7 | 5.86 | PASS | 8 | 7 | 6.13 | 0.96 | −1.05 | NDUFA9 | 12p | NADH dehydrogenase | NADH dehydrogenase |
| | | | | | | | | | | | | | | (ubiquinone) | (ubiquinone) 1 alpha |
| | | | | | | | | | | | | | | | subcomplex, 9 (39 kD) |
| STATH | U51678_{'}at | U51678 | PASS | 8 | 10.88 | PASS | 13 | 8 | 11.38 | 0.96 | −1.05 | | | small acidic protein | |
| X83416_s_at | X83416_s_at | X83416 | PASS | 7 | 6.00 | PASS | 13 | 7 | 6.31 | 0.95 | −1.05 | PRNP | 20pter-p12 | prion protein (p 27-30) | prion protein (p 27-30) |
| | | | | | | | | | | | | | | (Creutzfeld-Jacob disease, | (Creutzfeld-Jacob disease, |
| | | | | | | | | | | | | | | Gerstmann-Strausler- | Gerstmann-Strausler- |
| | | | | | | | | | | | | | | Scheinker syndrome, fatal | Scheinker syndrome, fatal |
| | | | | | | | | | | | | | | familial insomnia) | familial insomnia) |
| UBE2L1 | S81003_at | S81003 | PASS | 9 | 14.00 | PASS | 13 | 9 | 14.77 | 0.95 | −1.05 | UBE2L3 | 22q11.2 | ubiquitin-conjugating | ubiquitin-conjugating |
| | | | | | | | | | | | | | | enzyme E2L 3 | enzyme E2L 3 |
| EIF2A | J02645_at | J02645 | PASS | 5 | 7.80 | PASS | 8 | 5 | 8.25 | 0.95 | −1.06 | EIF2A | | eukaryotic translation | eukaryotic translation |
| | | | | | | | | | | | | | | initiation factor 2A | initiation factor 2A |
| DLD_rna1 | L13761_rna1 | L13761 | PASS | 9 | 6.67 | PASS | 12 | 9 | 7.08 | 0.94 | −1.06 | DLD | 7q31-q32 | dihydrolipoamide dehydro- | dihydrolipoamide dehydro- |
| | | | | | | | | | | | | | | genase (E3 component of | genase (E3 component of |
| | | | | | | | | | | | | | | pyruvate dehydrogenase | pyruvate dehydrogenase |
| | | | | | | | | | | | | | | complex, 2-oxo-glutarate | complex, 2-oxo-glutarate |
| | | | | | | | | | | | | | | complex, branched chain | complex, branched chain |
| | | | | | | | | | | | | | | keto acid dehydrogenase | keto acid dehydrogenase |
| | | | | | | | | | | | | | | complex) | complex) |
| OCLSF | U63717_at | U63717 | PASS | 7 | 7.00 | PASS | 8 | 7 | 7.50 | 0.93 | −1.07 | | | osteoclast stimulating | OSF; contains SH3 domain |
| | | | | | | | | | | | | | | factor | and ankyrin repeat |
| GNAO1 | U01833_at | U01833 | PASS | 5 | 5.60 | PASS | 9 | 5 | 6.00 | 0.93 | −1.07 | NBP1 | | nucleotide binding protein | nucleotide binding protein |
| | | | | | | | | | | | | | | 1 (E. coli MinD like) | 1 (E. coli MinD like) |
| M63838_s_at | M63838_s_at | M63838 | PASS | 9 | 12.33 | PASS | 13 | 9 | 13.23 | 0.93 | −1.07 | IF116 | 1q13-qter | interferon-gamma induced | interferon, gamma- |
| | | | | | | | | | | | | | | protein | inducible protein 16 |
| ADD3 | U37122_at | U37122 | PASS | 7 | 20.71 | PASS | 13 | 7 | 22.23 | 0.93 | −1.07 | ADD3 | 10q24.2-q24.3 | adducin gamma subunit | adducin 3 (gamma) |
| MLLT2 | L13773_at | L13773 | PASS | 9 | 6.00 | PASS | 12 | 9 | 6.50 | 0.92 | −1.08 | AF-4 | | | |
| Z69030_s_at | Z69030_s_at | Z69030 | PASS | 7 | 12.71 | PASS | 12 | 7 | 13.83 | 0.92 | −1.09 | | | gamma 1 isoform of 61 | |
| | | | | | | | | | | | | | | kDa regulatory subunit | |
| | | | | | | | | | | | | | | of PP2A | |
| T1G1_xpt1 | U49973_xpt1 | U49973 | PASS | 9 | 7.56 | PASS | 13 | 9 | 8.23 | 0.92 | −1.09 | | | | ORF1; MER37; putative |
| | | | | | | | | | | | | | | | transposase similar to |
| | | | | | | | | | | | | | | | pogo element |
| BTKAP135 | U77948_at | U77948 | PASS | 5 | 8.40 | PASS | 13 | 5 | 9.15 | 0.92 | −1.09 | GTF2I | 7q11.23 | general transcription | general transcription |
| | | | | | | | | | | | | | | factor, H, i | factor, H, i |
| HOU | U32849_at | U32849 | PASS | 9 | 10.00 | PASS | 12 | 9 | 10.92 | 0.92 | −1.09 | NMI | 22q13.3 | N-myc (and STAT) | N-myc (and STAT) |
| | | | | | | | | | | | | | | interactor | interactor |
| KLRB1 | HG4263-HT4 | HG4263-HT | PASS | 9 | 14.78 | PASS | 13 | 9 | 16.15 | 0.91 | −1.09 | | | | |
| AHNAK | M80899_at | M80899 | PASS | 7 | 18.14 | PASS | 12 | 7 | 19.83 | 0.91 | −1.09 | AHNAK | 11q12-q13 | | AHNAK nucleoprotein |
| | | | | | | | | | | | | | | | (desmoyokin) |
| NA | X80909_at | X80909 | PASS | 9 | 129.22 | PASS | 13 | 9 | 142.69 | 0.91 | −1.10 | alpha NAC | | Nascent polypeptide | |
| | | | | | | | | | | | | | | associated complex alpha | |
| | | | | | | | | | | | | | | subunit | |
| JAK1 | M64174_at | M64174 | PASS | 5 | 8.80 | PASS | 13 | 5 | 9.85 | 0.89 | −1.12 | JAK1 | 1p32.3-p31.3 | Janus kinase 1 (a protein | Janus kinase 1 (a protein |
| | | | | | | | | | | | | | | tyrosine kinase) | tyrosine kinase) |
| Z49501_s_at | Z48501_s_at | Z48501 | PASS | 9 | 113.67 | PASS | 13 | 9 | 127.77 | 0.89 | −1.12 | PABPL1 | 3q22-q25 | poly(A)-binding protein- | poly(A)-binding protein- |
| | | | | | | | | | | | | | | like 1 | like 1 |
| PRTKHT31 | HG2167-HT2 | HG2167-H | PASS | 5 | 7.00 | PASS | 9 | 5 | 7.89 | 0.89 | −1.13 | | | | |
| K192_MOPA | D83783_at | D83783 | PASS | 6 | 6.00 | PASS | 13 | 6 | 6.77 | 0.89 | −1.13 | TRAP230 | Xq13 | | thyroid hormone receptor- |
| | | | | | | | | | | | | | | protein, 230 kDa subunit | |
| PRF1 | M31951_at | M31951 | PASS | 6 | 23.33 | PASS | 12 | 6 | 26.67 | 0.88 | −1.14 | PRF1 | 10q22 | | perforin 1 (preforming |
| | | | | | | | | | | | | | | | protein) |
| PSMC2 | D11094_at | D11094 | PASS | 7 | 9.71 | PASS | 12 | 7 | 11.17 | 0.87 | −1.15 | PSMC2 | 7q22.1-q22.3 | proteasome (prosome, | proteasome (prosome, |
| | | | | | | | | | | | | | | macropain) 26S subunit, | macropain) 26S subunit, |
| | | | | | | | | | | | | | | ATPase, 2 | ATPase, 2 |
| SDHB | U17886_at | U17886 | PASS | 5 | 8.00 | PASS | 12 | 5 | 9.25 | 0.86 | −1.16 | sdhB | | succinate dehydrogenase | |
| | | | | | | | | | | | | | | iron-protein subunit B | |
| M21119_s_at | M21119_s_at | M21119 | PASS | 8 | 59.00 | PASS | 13 | 8 | 69.15 | 0.85 | −1.17 | | | | lysozyme precursor (EC |
| | | | | | | | | | | | | | | | 3.2.1.17) |
| S31I125 | L40397_at | L40397 | PASS | 9 | 10.78 | PASS | 13 | 9 | 12.77 | 0.84 | −1.18 | | | | ORF; putative |
| RNASE6 | U64998_at | U64998 | PASS | 6 | 13.33 | PASS | 10 | 6 | 15.80 | 0.84 | −1.19 | | | ribonuclease k6 precursor | RNase k6 |
| IK | S74221_at | S74221 | PASS | 8 | 10.13 | PASS | 12 | 8 | 12.08 | 0.84 | −1.19 | IK | 2p15-p14 | IK cytokine, down- | IK cytokine, down- |
| | | | | | | | | | | | | | | regulator of HLA II | regulator of HLA II |
| U73477_{'}s_at | U73477_s_at | U73477 | PASS | 6 | 6.00 | PASS | 10 | 6 | 7.20 | 0.83 | −1.20 | | | acidic nuclear phospho- | LANP; PHAPI; I-1pp2a |
| | | | | | | | | | | | | | | protein pp32 | |
| M97796_s_at | M97796_s_at | M97796 | PASS | 6 | 17.83 | PASS | 13 | 6 | 21.62 | 0.83 | −1.21 | ID2 | 2p25 | inhibitor of DNA binding 2, | inhibitor of DNA binding 2, |
| | | | | | | | | | | | | | | dominant negative helix- | dominant negative helix- |
| | | | | | | | | | | | | | | loop-helix protein | loop-helix protein |
| NIP2 | U15173_at | U15173 | PASS | 8 | 4.88 | PASS | 12 | 8 | 5.92 | 0.82 | −1.21 | VNIP2 | | BCL2/adenovirus E1B 19 | BCL2/adenovirus E1B 19 |
| | | | | | | | | | | | | | | kD-interacting protein 2 | kD-interacting protein 2 |
| POLR2B | X63563_at | X63563 | PASS | 7 | 6.71 | PASS | 12 | 7 | 8.17 | 0.82 | −1.22 | POLR2B | 4q12 | polymerase (RNA) II | polymerase (RNA) II |
| | | | | | | | | | | | | | | (DNA directed) polypeptide | (DNA directed) polypeptide |
| | | | | | | | | | | | | | | B (140 kD) | B (140 kD) |
| AFX-CreX- | AFFX-CreX- | AFFX-CreX | PASS | 8 | 15.38 | PASS | 13 | 8 | 18.77 | 0.82 | −1.22 | | | | |
| TRAMP | X63679_at | X63679 | PASS | 6 | 8.50 | PASS | 13 | 6 | 10.38 | 0.82 | −1.22 | TRAM | | TRAM protein | |
| SAP18 | U96915_at | U96915 | PASS | 8 | 13.13 | PASS | 13 | 8 | 16.08 | 0.82 | −1.22 | SAP18 | | sin3 associated poly- | SAP 18p |
| | | | | | | | | | | | | | | | peptide p18 |
| BIOC5 | AFFX-BioC- | AFFX-BioC | PASS | 9 | 16.00 | PASS | 13 | 9 | 19.62 | 0.82 | −1.23 | | | | |
| RB1 | HG4036-HT4 | HG4036-HT | PASS | 8 | 11.13 | PASS | 13 | 8 | 14.31 | 0.78 | −1.29 | | | | |
| TFA | L06633_at | L06633 | PASS | 8 | 6.75 | PASS | 13 | 8 | 8.85 | 0.76 | −1.31 | HE | 2 | cytohesin binding protein | cytohesin binding protein |
| | | | | | | | | | | | | | | HE | HE |
| IL7R | M29696_at | M29696 | PASS | 8 | 26.38 | PASS | 11 | 8 | 34.73 | 0.76 | −1.32 | IL7R | 5p13 | interleukin 7 receptor | interleukin 7 receptor |
| ZFP20 | HG3454-HT3 | HG3454-HT | PASS | 7 | 5.43 | PASS | 9 | 7 | 7.22 | 0.75 | −1.33 | | | | |
| COPB | X70476_at | X70476 | PASS | 8 | 6.38 | PASS | 13 | 8 | 8.92 | 0.71 | −1.40 | COPB2 | 3q23 | coatomer protein complex, | coatomer protein complex, |
| | | | | | | | | | | | | | | subunit beta 2 (beta prime) | subunit beta 2 (beta prime) |
| RAP1B | HG3521-HT3 | HG3521-HT | PASS | 9 | 17.56 | PASS | 13 | 9 | 25.62 | 0.69 | −1.46 | | | | |
| BIOB3 | AFFX-BioB-3 | AFFX-BioB | PASS | 9 | 27.11 | PASS | 13 | 9 | 39.77 | 0.68 | −1.47 | | | | |
| PROGBP | Y12711_at | Y12711 | PASS | 6 | 9.33 | PASS | 13 | 6 | 14.00 | 0.67 | −1.50 | | | putative progesterone | |
| | | | | | | | | | | | | | | binding protein | |
| M27394_s_at | M27394_s_at | M27394 | PASS | 6 | 6.00 | PASS | 11 | 6 | 9.73 | 0.62 | −1.62 | | | cell surface antigen B1 | |
| DAP3 | AFFX-DapX- | AFFX-DapX | PASS | 9 | 6.56 | PASS | 13 | 9 | 10.69 | 0.61 | −1.63 | | | | |
| M14483_rna1 | M14483_rna1 | M14483 | PASS | 9 | 65.89 | PASS | 13 | 9 | 109.31 | 0.60 | −1.66 | PTMA | 2 | prothymosin, alpha (gene | prothymosin, alpha (gene |
| | | | | | | | | | | | | | | sequence 28) | sequence 28) |
| BIOD3 | AFFX-BioDn | AFFX-BioD | PASS | 9 | 29.56 | PASS | 13 | 9 | 65.62 | 0.45 | −2.22 | | | | |

TABLE 2
6800 human RA Phosphatase and kinase list

| | | | | | | | | | | | | | | | |
|---|---|---|---|---|---|---|---|---|---|---|---|---|---|---|---|
| | | | Patients | | | Normals | | | | | | | | | |
| | | GeneSpring | called | #“P” | Avg Freq- | called | #“P” | #“P” | Avg Freq | | Fold | | Chromo- | | Kinase or |
| name | qualifier | qualifier | “P”>4 | (RA) | RA Patients | “P”>6 | (Normal) | (RA) | Normals | Ratio | Change | Symbol | some | Description | Phosphatase |

Kinases

| | | | | | | | | | | | | | | | | |
|---|---|---|---|---|---|---|---|---|---|---|---|---|---|---|---|---|
| RAC2 | M64595 at | M64595 | fail | 3 | | PASS | 13 | 3 | 19.85 | Normal | Normal | RAC2 | 22q12-13 2 | ras-related C3 botulinum toxin | Kinase | |
| | | | | | | | | | | | | | | substrate 2 (rho family, small GTP | | |
| | | | | | | | | | | | | | | binding protein Rac2) | | |
| FRAP | L34075_at | L34075 | fail | 1 | | PASS | 11 | 1 | 5.73 | Normal | Normal | FRAP1 | 1p36.2 | FKBP-rapamycin associated | Kinase | |
| | | | | | | | | | | | | | | protein | | |
| CAMKA2 | U81554_at | U81554 | fail | 1 | | PASS | 10 | 1 | 5.50 | Normal | Normal | CAMK2G | 10q22 | calcium/calmodulin-depenent | Kinase | |
| | | | | | | | | | | | | | | protein kinase (CaM kinase) II | | |
| | | | | | | | | | | | | | | gamma | | |
| CDK7 | L20320_at | L20320 | fail | 1 | | PASS | 9 | 1 | 4.89 | Normal | Normal | CDK7 | 2p15-cen | cyclin-dependent kinase 7 | Kinase | |
| | | | | | | | | | | | | | | (homolog of Xenopus MO15 cdk- | | |
| | | | | | | | | | | | | | | activating kinase) | | |
| EPHB4 | U07695_at | U07695 | fail | 0 | | PASS | 8 | 0 | 6.50 | Normal | Normal | EPHB4 | 7 | EphB4 | Kinase | |
| PRP4H | U48736_at | U48736 | fail | 0 | | PASS | 8 | 0 | 5.00 | Normal | Normal | PRP4 | | serine/threonine-protein kinase | Kinase | |
| | | | | | | | | | | | | | | PRP4 homolog | | |
| BLK | S76617_at | S76617 | fail | 0 | | PASS | 7 | 0 | 4.71 | Normal | Normal | BLK | 8p23-p22 | B lymphoid tyrosine kinase | Kinase | |
| CHED M80629_at | M80629 | fail | 3 | | PASS | 10 | 3 | 5.30 | Normal | Normal | CDC2L | | cholinesterase-related cell division | Kinase | | |
| | | | | | | | | | | | | | | controller | | |
| CLC | L01664_at | L01664 | fail | 3 | | PASS | 9 | 3 | 7.22 | Normal | Normal | CLC | 19q13.1 | Charot-Leyden crystal protein | Kinase | |
| L05624 s g | L05624_s_{—l at} | L05624 | fail | 3 | | PASS | 9 | 3 | 5.56 | Normal | Normal | | | MAP kinase kinase | Kinase | |
| PLK | U01038_at | U01038 | fail | 3 | | PASS | 9 | 3 | 5.56 | Normal | Normal | | | pLK | Kinase | |
| S80267_{—} | S80267_s_at | S80267 | fail | 4 | | PASS | 8 | 4 | 4 88 | Normal | Normal | p72syk | | p72syk | Kinase | |
| s_g | | | | | | | | | | | | | | | | |
| CSNK1A1 | L37042_at | L37042 | fail | 4 | | PASS | 7 | 4 | 8 00 | Normal | Normal | CSNK1A1 | 13q13 | caseine kinase 1, alpha 1 | Kinase | |
| CSNK2A1 | M55265_at | M55265 | fail | 3 | | PASS | 7 | 3 | 5.86 | Normal | Normal | CSNK2A1 | 20p13 | casein kinase 2, alpha 1 polypeptide | Kinase | |
| HG4120-H | HG4120-HT | HG4120-HT | PASS | 6 | 5.17 | fail | 3 | 6 | | Disease | Disease | | | | Kinase | |
| LTK | D16105_at | D16105 | PASS | 9 | 23.78 | fail | 3 | 9 | | Disease | Disease | LTK | 15 | leukocyte tyrosine kinase | Kinase | |
| K60_GNPT | D31766_at | D31766 | PASS | 8 | 14.50 | fail | 6 | 8 | | Disease | Disease | KIAA0060 | | KIAA0060 gene product | Kinase | |
| CDK2 | M68520_at | M68520 | PASS | 8 | 7.63 | fail | 6 | 8 | | Disease | Disease | CDK2 | 12q13 | cyclin-dependent kinase 2 | Kinase | |
| PRXACG | M34182 at | M34182 | PASS | 8 | 41.00 | fail | 5 | 8 | | Disease | Disease | PRKACG | 9q13 | protein kinase, cAMP-dependent, | Kinase | |
| | | | | | | | | | | | | | | catalytic, gamma | | |
| K213 | D86968_at | D86968 | PASS | 7 | 4.14 | fail | 6 | 7 | | Disease | Disease | KIAA0213 | | Similar to Mouse TFIIi-associated | Kinase | |
| | | | | | | | | | | | | | | transactivator factor | | |
| | | | | | | | | | | | | | | p17(GB_RO:MMU11548): | | |
| | | | | | | | | | | | | | | Containing protein kinase motif | | |
| TESK1 | D50863_at | D50863 | PASS | 5 | 8.80 | fail | 6 | 5 | | Disease | Disease | TESK1 | 9p13 | TESK1, testis-specific kinase 1 | Kinase | |
| GCDH | U69141_at | U69141 | PASS | 5 | 6.40 | fail | 4 | 5 | | Disease | Disease | GCDH | 19p13.2 | glutaryl-Coenzyme A | Kinase | |
| | | | | | | | | | | | | | | dehydrogenase | | |
| ILK | U40282_at | U40282 | PASS | 9 | 29 22 | PASS | 12 | 9 | 8.58 | 3.40 | 3 40 | ILK | 11p15.5- | integrin-linked kinase | Kinase | |
| | | | | | | | | | | | | | p15.4 | | | |
| HCFC1 | L20010_at | L20010 | PASS | 8 | 26.13 | PASS | 13 | 8 | 8.92 | 2.93 | 2.93 | | | | Kinase | |
| PRKMK3 | D87116_at | D87116 | PASS | 9 | 32.89 | PASS | 11 | 9 | 11.27 | 2.92 | 2 92 | PRKMK3 | 17q11.2 | protein kinase, mitrogen-activated, | Kinase | |
| | | | | | | | | | | | | | | kinase 3 (MAP kinase kinase 3) | | |
| FAST | X86779_at | X86779 | PASS | 9 | 20.33 | PASS | 10 | 9 | 7.30 | 2 79 | 2 79 | fast | | FAST kinase | Kinase | |
| X59932_s_{—} | X59932_s_a | X59932 | PASS | 9 | 62.44 | PASS | 13 | 9 | 22 46 | 2.78 | 2 78 | CSK | 15q23-q25 | c-src tyrosine kinase | Kinase | |
| CSNK2A2 | M55268_at | M55268 | PASS | 9 | 17.33 | PASS | 7 | 9 | 6.57 | 2.64 | 2.64 | CSNK2A2 | 16p13 3- | casein kinase 2, alpha prime | Kinase | |
| | | | | | | | | | | | | | p13.2 | polypeptide | | |
| K151_SPK | D63485_at | D63485 | PASS | 9 | 18.89 | PASS | 10 | 9 | 7.20 | 2.62 | 2.62 | KIAA0151 | | KIAA0151 gene product | Kinase | |
| M16750_{—l s} | M16750 s a | M16750 | PASS | 9 | 34.89 | PASS | 13 | 9 | 13.92 | 2.51 | 2.51 | PIM1 | 6p21 | pim-1 oncogene | Kinase | |
| RPS6KA2 | L07597_at | L07597 | PASS | 9 | 28.78 | PASS | 12 | 9 | 11.92 | 2.41 | 2.41 | RPS6KA1 | 3 | ribosomal protein S6 kinase, 90kD, | Kinase | |
| | | | | | | | | | | | | | | polypeptide 1 | | |
| ECGF1_rna | U62317_rna | U62317 | PASS | 9 | 66 22 | PASS | 13 | 9 | 27.54 | 2.40 | 2.40 | | | arylsulfatase A | Kinase | |
| GLA | X14448_at | X14448 | PASS | 9 | 20.56 | PASS | 13 | 9 | 8.62 | 2.39 | 2.39 | | | alpha-D-galactosidase A | Kinase | |
| DGK5Z | U51477_at | U51477 | PASS | 9 | 32.56 | PASS | 13 | 9 | 13.77 | 2.36 | 2.36 | DGKZ | | diacylglycerol kinase, zeta (104D) | Kinase | |
| PIM2 | U77735_at | U77735 | PASS | 6 | 24.33 | PASS | 12 | 6 | 10.33 | 2.35 | 2 35 | | | pim-2 protooncogene homolog pim- | Kinase | |
| | | | | | | | | | | | | | | 2h | | |
| M54915_s_{—} | M54915_s_a | M54915 | PASS | 9 | 54.67 | PASS | 13 | 9 | 23.54 | 2.32 | 2.32 | | | pim-1 protein | Kinase | |
| CAKB | U43522_at | U43522 | PASS | 8 | 14 13 | PASS | 9 | 8 | 6.11 | 2.31 | 2.31 | PTK2B | 8p21 1 | focal adhesion kinase 2 (protein | Kinase | |
| | | | | | | | | | | | | | | kinase B) | | |
| M13829_s_{—} | M13829_s_a | M13829 | PASS | 8 | 15 25 | PASS | 13 | 8 | 6.69 | 2.28 | 2.28 | ARAF1 | Xp11.4- | v-raf murine sarcoma 3611 viral | Kinase | |
| | | | | | | | | | | | | | p11.2 | oncogene homolog 1 | | |
| AKT1 | M63167_at | M63167 | PASS | 8 | 23.13 | PASS | 11 | 8 | 10 18 | 2.27 | 2 27 | AKT1 | 14q32.3 | rac protein kinase-alpha | Kinase | |
| ZAP70 | L05148_at | L05148 | PASS | 9 | 36.56 | PASS | 13 | 9 | 16.31 | 2.24 | 2 24 | | | | Kinase | |
| RAD23A | D21235_at | D21235 | PASS | 9 | 15.56 | PASS | 10 | 9 | 7.00 | 2.22 | 2.22 | RAD23A | 19p13.2 | HHR23A protein,RAD23 (S. | Kinase | |
| | | | | | | | | | | | | | | cerevisiae) homolog A | | |
| FGR | M19722_at | M19722 | PASS | 9 | 94 78 | PASS | 13 | 9 | 43.00 | 2.20 | 2.20 | FGR | 1p36 2- | Gardner-Rasheed feline sarcoma | Kinase | |
| | | | | | | | | | | | | | p36.1 | viral (v-fgr) oncogene homolog | | |
| DAGK1 | X62535_at | X62535 | PASS | 9 | 38.56 | PASS | 13 | 9 | 17.92 | 2 15 | 2 15 | DGKA | 12 | diacylglycerol kinase | Kinase | |
| C8FWPH | AJ000480_at | AJ000480 | PASS | 5 | 11.60 | PASS | 9 | 5 | 5.44 | 2.13 | 2.13 | C8FW | | phosphoprotein | Kinase | |
| D63479_s_{—} | D63479_s_a | D63479 | PASS | 9 | 18.67 | PASS | 12 | 9 | 8 83 | 2.11 | 2 11 | DGKD | | diacylglycerol kinsae, delta | Kinase | |
| | | | | | | | | | | | | | | (130kD) | | |
| HPK1 | U66464_at | U66464 | PASS | 9 | 16 89 | PASS | 13 | 9 | 8.00 | 2.11 | 2.11 | HPK1 | | hematopoietic progenitor kinase | Kinase | |
| GPRK6 | L16862_at | L16862 | PASS | 7 | 25 29 | PASS | 7 | 7 | 12.00 | 2.11 | 2.11 | GPRK6 | 5q35 | G protein-coupled receptor kinsae 6 | Kinase | |
| IRAK1 | L76191_at | L76191 | PASS | 9 | 32.67 | PASS | 13 | 9 | 15 54 | 2 10 | 2.10 | IRAK1 | Xq28 | interleukin-1 receptor-associated | Kinase | |
| | | | | | | | | | | | | | | kinase 1 | | |
| PI4KB | U81802_{—l at} | U81802 | PASS | 7 | 14.71 | PASS | 11 | 7 | 7 00 | 2 10 | 2 10 | PIK4CB | 1q21 | phosphatidylinositol 4-kinase, | Kinase | |
| | | | | | | | | | | | | | | catalytic, beta polypeptide | | |
| Z69043_s_{—} | Z69043_s_{—l at} | Z69043 | PASS | 9 | 57 11 | PASS | 13 | 9 | 27.62 | 2.07 | 2.07 | H-TRAP | | translocon-associated protein delta | Kinase | |
| a | | | | | | | | | | | | | | delta | | subunit precursor |
| GSK3A | L40027_at | L40027 | PASS | 9 | 19.89 | PASS | 13 | 9 | 9.77 | 2.04 | 2 04 | | | glycogen synthase kinase 3 | Kinase | |
| PIK4 | L36151_at | L36151 | PASS | 9 | 39.89 | PASS | 13 | 9 | 19 69 | 2.03 | 2.03 | PIK4CA | | phosphatidylinositol 4-kinase, | Kinase | |
| | | | | | | | | | | | | | | catalytic, alpha polypeptide | | |
| 3PK | U09578_at | U09578 | PASS | 8 | 17.25 | PASS | 13 | 8 | 8.54 | 2.02 | 2.02 | MAPKAPK3 | 3p21.3 | mitogen-activated protein kinase- | Kinase | |
| | | | | | | | | | | | | | | activated protein kinase 3 | | |
| MLK3 | L32976_at | L32976 | PASS | 7 | 12.29 | PASS | 7 | 7 | 6.14 | 2.00 | 2.00 | MLK3 | 11q13.1- | mixed lineage kinase 3 | Kinase | |
| | | | | | | | | | | | | | q13.3 | | | |
| CLK2 | L29218_at | L29218 | PASS | 7 | 13.71 | PASS | 11 | 7 | 7.09 | 1.93 | 1.93 | CLK2 | 1q21 | CDC-like kinase 2 | Kinase | |
| K135_PIM | D50925_at | D50925 | PASS | 6 | 12.00 | PASS | 9 | 6 | 6.33 | 1.89 | 1.89 | KIAA0135 | | The KIAA0135 gene as related to | Kinase | |
| | | | | | | | | | | | | | | pim-1 oncogene. | | |
| ZAP112 | L40399_at | L40399 | PASS | 6 | 26.50 | PASS | 10 | 6 | 14 00 | 1 89 | 1.89 | | | ORF, putative | Kinase | |
| CBOGS395 | D87119_at | D87119 | PASS | 8 | 19.75 | PASS | 12 | 8 | 10.50 | 1.88 | 1 88 | | | GS3955 | Kinase | |
| PRKCD | D10495_at | D10495 | PASS | 9 | 30.22 | PASS | 12 | 9 | 16.58 | 1 82 | 1.82 | 35550 | | protein kinase C delta-type | Kinase | |
| CSNK1D | U29171_at | U29171 | PASS | 9 | 18 22 | PASS | 12 | 9 | 10 08 | 1.81 | 1.81 | CSNK1D | 17q25 | casein kinase 1, delta | Kinase | |
| K96_PK | D43636_at | D43636 | PASS | 9 | 15.89 | PASS | 13 | 9 | 9.00 | 1.77 | 1.77 | KIAA0096 | | KIAA0096 gene product is related | Kinase | |
| | | | | | | | | | | | | | | to a protein kinase. | | |
| FYN | M14676_at | M14676 | PASS | 9 | 31.89 | PASS | 13 | 9 | 18 08 | 1.76 | 1.76 | FYN | 6q21 | FYN oncogene related to SRC, | Kinase | |
| | | | | | | | | | | | | | | FGR, YES | | |
| CSNK1G2 | U89896_at | U89896 | PASS | 6 | 9.83 | PASS | 9 | 6 | 5.67 | 1.74 | 1.74 | | | casein kinase 1 gamma 2 | Kinase | |
| TRNASTL | U07424_at | U07424 | PASS | 7 | 14.00 | PASS | 11 | 7 | 8.09 | 1.73 | 1.73 | FARSL | | phenylalamine-tRNA synthetase- | Kinase | |
| | | | | | | | | | | | | | | like | | |
| PKUA | AB004884_a | AB004884 | PASS | 7 | 12.29 | PASS | 8 | 7 | 7.13 | 1.72 | 1.72 | | | PKU-alpha | Kinase | |
| U23852_s_{—} | U23852_s_a | U23852 | PASS | 9 | 66.89 | PASS | 13 | 9 | 38.85 | 1.72 | 1.72 | 1ck | | p561ck | Kinase | |
| DYRK | D86550_at | D86550 | PASS | 9 | 18.67 | PASS | 13 | 9 | 10.85 | 1.72 | 1.72 | hMNB | | serine/threonine protein kinase | Kinase | |
| SLC6A8_m | U36341_ma | U36341 | PASS | 9 | 13.11 | PASS | 9 | 9 | 7.67 | 1.71 | 1.71 | SLC6A8 | | creatine transporter | Kinase | |
| SSBP | M94556_at | M94556 | PASS | 9 | 23.78 | PASS | 13 | 9 | 14.15 | 1.68 | 1.68 | SSBP | 7q34 | single-stranded DNA-binding | Kinase | |
| | | | | | | | | | | | | | | protein | | |
| D13720_s_{—} | D13720_s_a | D13720 | PASS | 8 | 14.25 | PASS | 13 | 8 | 8.62 | 1.65 | 1.65 | | | ITK | Kinase | |
| ABR | U01147_at | U01147 | PASS | 9 | 9.89 | PASS | 12 | 9 | 6 00 | 1.65 | 1.65 | ABR | 17p13.3 | guanine nucleotide regulatory | Kinase | |
| | | | | | | | | | | | | | | protein | | |
| M16591_s_{—} | M16591_s_a | M16591 | PASS | 9 | 29.44 | PASS | 13 | 9 | 17 92 | 1 64 | 1.64 | HCK | 20q11-q12 | hemopoietic cell kinase | Kinase | |
| X77588_s_{—} | X77588_s_a | X77588 | PASS | 8 | 10.88 | PASS | 12 | 8 | 6.67 | 1.63 | 1.63 | ARDI | Xq28 | ARDI N-acetyl transferase | Kinase | |
| | | | | | | | | | | | | | | homologue | | |
| TFE3_ma1 | X97160_ma | X97160 | PASS | 7 | 9.29 | PASS | 7 | 7 | 6.00 | 1.55 | 1.55 | | | TFE3 transcription factor | Kinase | |
| M30448_s_{—} | M30448_s_a | M30448 | PASS | 9 | 58.11 | PASS | 13 | 9 | 38.38 | 1.51 | 1.51 | CSNK2B | 6p21-p12 | casein kinase 2, beta polypeptide | Kinase | |
| K137_COS | D50927_at | D50927 | PASS | 8 | 10 25 | PASS | 13 | 8 | 6.85 | 1.50 | 1 50 | KIAA0137 | | KIAA0137 gene product | Kinase | |
| PAK1 | U24152_at | U24152 | PASS | 9 | 13 22 | PASS | 12 | 9 | 8.83 | 1.50 | 1 50 | PAK1 | 11q13-q14 | p21/Cdc42/Rac1-activated kinase 1 | Kinase | |
| | | | | | | | | | | | | | | (yeast Ste20-related) | | |
| ZPK | U07358_at | U07358 | PASS | 7 | 8.86 | PASS | 11 | 7 | 6 09 | 1.45 | 1.45 | ZPK | 12q13 | serine/threonine protein kinase | Kinase | |
| PSMD10_c | X71874_cds | X71874 | PASS | 9 | 73.89 | PASS | 13 | 9 | 51.23 | 1 44 | 1.44 | PSMB10 | 16q22.1 | proteasome (prosome, macropain) | Kinase | |
| | | | | | | | | | | | | | | subunit, beta type, 10 | | |
| M36430_s_{—} | M36430_s_a | M36430 | PASS | 7 | 12.71 | PASS | 13 | 7 | 8 85 | 1 44 | 1.44 | GNB1 | 1p36.21- | guanine nucleotide binding protein | Kinase | |
| | | | | | | | | | | | | | | (G protein), beta polypeptide 1 | | |
| RPS6KA2 | U08316_at | U08316 | PASS | 5 | 8.00 | PASS | 11 | 5 | 5.64 | 1.42 | 1.42 | RPS6KA3 | Xp22 2- | ribosomal protein S6 kinase, 90kD, | Kinase | |
| | | | | | | | | | | | | | p22.1 | polypeptide 3 | | |
| ATP7A | AB000409_a | AB000409 | PASS | 9 | 9.89 | PASS | 10 | 9 | 7.10 | 1.39 | 1.39 | MKNK1 | MAP kinase-interacting | Kinase | | |
| | | | | | | | | | | | | | | serine/threonine kinase 1 | | |
| HG3730-H | HG3730-HT | HG3730-HT | PASS | 8 | 9.75 | PASS | 12 | 8 | 7 08 | 1.38 | 1.38 | | | | Kinase | |
| LYN | M16038 at | M16038 | PASS | 9 | 17.11 | PASS | 13 | 9 | 12 54 | 1 36 | 1.36 | LYN | 8q13 | v-yes-1 Yamaguchi sarcoma viral | Kinase | |
| | | | | | | | | | | | | | | related oncogene homolog | | |
| D26535_s_{—} | D26535_s_a | D26535 | PASS | 8 | 10.00 | PASS | 11 | 8 | 7.36 | 1.36 | 1.36 | DLST | 14q24.3 | dihydrolipoamide S- | Kinase | |
| | | | | | | | | | | | | | | succinyltransferase (E2 component | | |
| | | | | | | | | | | | | | | of 2-oxo-glutarate complex) | | |
| DYRK2 | Y09216_at | Y09216 | PASS | 6 | 10.17 | PASS | 12 | 6 | 7.50 | 1.36 | 1.36 | DYRK2 | 12 | dual-specificity tyrosine-(Y)- | Kinase | |
| | | | | | | | | | | | | | | phosphorylation regulated kinase 2 | | |
| BTK_ma4 | U78027_ma | U78027 | PASS | 7 | 10.71 | PASS | 12 | 7 | 7.92 | 1.35 | 1.35 | FTP3 | | FTP3 | Kinase | |
| PSKH1 | U09564_at | U09564 | PASS | 8 | 7.50 | PASS | 11 | 8 | 5.55 | 1.35 | 1.35 | SRPK1 | 6p21.2- | SFRS protein kinase 1 | Kinase | |
| | | | | | | | | | | | | | p21.3 | | | |
| VRK1 | AB000449_a | AB000449 | PASS | 7 | 7 57 | PASS | 10 | 7 | 5.90 | 1.28 | 1.28 | VRK1 | 14q32 | vaccinia related kinase 1 | Kinase | |
| HNRNPCL | M94630_at | M94630 | PASS | 9 | 27.56 | PASS | 13 | 9 | 21.69 | 1.27 | 1.27 | HNRPD | 4q21 | heterogeneous nuclear | Kinase | |
| | | | | | | | | | | | | | | ribonucleoprotein D | | |
| TGFBR2 | D50683_at | D50683 | PASS | 9 | 24.00 | PASS | 13 | 9 | 18.92 | 1.27 | 1.27 | TGFBR2 | 3p22 | transforming growth factor, beta | Kinase | |
| | | | | | | | | | | | | | | receptor II (70-80kD) | | |
| GPRK5 | L15388_at | L15388 | PASS | 6 | 6.33 | PASS | 7 | 6 | 5.00 | 1.27 | 1.27 | GPRK5 | 10q24-qter | G protein-coupled receptor kinase | Kinase | |
| HG3484-H | HG3484-HT | HG3484-HT | PASS | 7 | 8.86 | PASS | 12 | 7 | 7.00 | 1.27 | 1.27 | | | | Kinase | |
| ATM | U33841_at | U33841 | PASS | 5 | 6.20 | PASS | 9 | 5 | 5.11 | 1.21 | 1.21 | ATM | 11q22-q23 | ataxia telangiectasia mutated | Kinase | |
| | | | | | | | | | | | | | | (includes complementation groups | | |
| | | | | | | | | | | | | | | A, C and D) | | |
| DNAPKCS | U47077_at | U47077 | PASS | 7 | 5 29 | PASS | 8 | 7 | 4.38 | 1.21 | 1.21 | PRKDC | 8q11 | DNA-dependent protein kinase | Kinase | |
| | | | | | | | | | | | | | | catalytic subunit | | |
| YES1 | M15990_at | M15990 | PASS | 7 | 5.43 | PASS | 7 | 7 | 4.71 | 1.15 | 1.15 | YES1 | 18p11.31- | v-yes-1 Yamaguchi sarcoma viral | Kinase | |
| | | | | | | | | | | | | | p11.21 | oncogene homolog 1 | | |
| PRKMK1 | L11284_at | L11284 | PASS | 7 | 10 29 | PASS | 13 | 7 | 9.46 | 1 09 | 1.09 | PRKMK1 | 15q22.1- | protein kinase, mitogen-activated, | Kinase | |
| | | | | | | | | | | | | | q22 33 | | kinase 1 (MAP kinase kinase 1) | |
| S203_15 | L40395_at | L40395 | PASS | 8 | 6.50 | PASS | 9 | 8 | 6.11 | 1.06 | 1.06 | | | ORF; putative | Kinase | |
| SRPK2 | U88666_at | U88666 | PASS | 8 | 5.38 | PASS | 12 | 8 | 5.08 | 1.06 | 1.06 | SRPK2 | 7q22-q31.1 | SFRS protein kinase 2 | Kinase | |
| PAK3 | U25975_at | U25975 | PASS | 5 | 5.60 | PASS | 10 | 5 | 5.40 | 1.04 | 1.04 | hPAK65 | | hPAK65 | Kinase | |
| PRKACB | M34181_at | M34181 | PASS | 5 | 6.80 | PASS | 10 | 5 | 6.70 | 1.01 | 1.01 | PRKACB | 1 | protein kinase, cAMP-dependent, | Kinase | |
| | | | | | | | | | | | | | | catalytic, beta | | |
| JAK1 | M64174_at | M64174 | PASS | 5 | 8.80 | PASS | 13 | 5 | 9.85 | 0.89 | −1.12 | JAK1 | 1p32.3- | Janus kinase 1 (a protein tyrosine | Kinase | |
| | | | | | | | | | | | | | p31.3 | kinase) | | |

| | | Phosphatases | | | | | | | | | | | | | |
|---|---|---|---|---|---|---|---|---|---|---|---|---|---|---|---|
| PTPRA | M34668_at | M34668 | fail | 2 | | PASS | 9 | 2 | 5.67 | Normal | Normal | PTPRA | 20p13 | protein tyrosine phosphatase, | Phosphatase |
| | | | | | | | | | | | | | | receptor type, alpha polypeptide | |
| PTEN | U92436_at | U92436 | fail | 3 | | PASS | 11 | 3 | 5.00 | Normal | Normal | PTEN | 10q23 | phosphatase and tensin homolog | Phosphatase |
| | | | | | | | | | | | | | | (mutated in multiple advanced | |
| | | | | | | | | | | | | | | cancers 1) | |
| PLCD1 | U09117_at | U09117 | fail | 4 | | PASS | 7 | 4 | 7 57 | Normal | Normal | | | phospholipase c delta 1 | Phosphatase |
| PTPRE | HG620-HT6 | HG620-HT6 | fail | 3 | | PASS | 7 | 3 | 8 00 | Normal | Normal | | | | Phosphatase |
| PLCG2H | U45974_at | U45974 | PASS | 5 | 15.40 | fail | 0 | 5 | | Disease | Disease | | | | Phosphatase |
| PTPRN | L18983_{—l at} | L18983 | PASS | 5 | 20.00 | fail | 0 | 5 | | Disease | Disease | PTPRN | 2q35-q36.1 | protein tyrosine phosphatase, | Phosphatase |
| | | | | | | | | | | | | | | receptor type, N | |
| INPPL1 | L36818_at | L36818 | PASS | 7 | 21.71 | fail | 6 | 7 | | Diseaae | Disease | | | 51C protein | Phosphatase |
| M33684_s_{—} | M33684_s_a | M33684 | PASS | 5 | 6.60 | fail | 3 | 5 | | Disease | Disease | PTPN1 | | non-receptor tyrosine phosphatase | Phosphatase |
| | | | | | | | | | | | | | | 1 | |
| PLCB2 | M95678_at | M95678 | PASS | 9 | 84.00 | PASS | 12 | 9 | 26.92 | 3.12 | 3.12 | PLCB2 | 15q15 | phospholipase C, beta 2 | Phosphatase |
| K15_PPM1 | D13640_at | D13640 | PASS | 9 | 29.00 | PASS | 12 | 9 | 11.33 | 2.56 | 2.56 | KIAA0015 | | | Phosphatase |
| PPP4C | X70218_at | X70218 | PASS | 7 | 27.43 | PASS | 11 | 7 | 11.18 | 2.45 | 2.45 | PP4C | 16p12- | protein phosphatase 4 (formerly X), | Phosphatase |
| | | | | | | | | | | | | | 16p11 | catalytic subunit | |
| INPP5D | U57650_at | U57650 | PASS | 9 | 42.78 | PASS | 13 | 9 | 18.77 | 2.28 | 2.28 | INPP5D | 2q36-q37 | SH2-containing inositol 5- | Phosphatase |
| | | | | | | | | | | | | | | phosphatase | |
| PP1 | U14603_at | U14603 | PASS | 9 | 76.56 | PASS | 13 | 9 | 35.31 | 2.17 | 2.17 | PTP4A2 | 1p35 | protein tyrosine phosphatase type | Phosphatase |
| | | | | | | | | | | | | | | IVA, member 2 | |
| J03805_s_a | J03805_s_{—l at} | J03805 | PASS | 9 | 14.33 | PASS | 13 | 9 | 7.23 | 1.98 | 1.98 | PPP2CB | 8p12-p11.2 | protein phosphatase 2 (formerly | Phosphatase |
| | | | | | | | | | | | | | | 2A), catalytic subunit, beta isoform | |
| M37238_s_{—} | M37238_s_a | M37238 | PASS | 9 | 11.33 | PASS | 9 | 9 | 6.22 | 1.82 | 1 82 | PLCG2 | 16q24.1 | phospholipase C, gamma 2 | Phosphatase |
| | | | | | | | | | | | | | | (phosphatidylinositol-specific) | |
| PTPCAAX | U48296_at | U48296 | PASS | 7 | 8.43 | PASS | 8 | 7 | 5 13 | 1 64 | 1.64 | PTP4A1 | 6q12 | Protein tyrosine phoshatase IVA1 | Phosphatase |
| PPP3CB | S46622_at | S46622 | PASS | 5 | 6.40 | PASS | 10 | 5 | 4.40 | 1.45 | 1.45 | calcineurin A | | calcineurin A catalytic subunit | Phosphatase |
| | | | | | | | | | | | | catalytic | | | |
| | | | | | | | | | | | | subunit, | | | |
| | | | | | | | | | | | | calmodulin- | | | |
| | | | | | | | | | | | | dependent | | | |
| | | | | | | | | | | | | protein | | | |
| | | | | | | | | | | | | phosphatase | | | |
| | | | | | | | | | | | | catalytic | | | |
| | | | | | | | | | | | | subunit, CaM- | | | |
| PTPN12 | M93425_at | M93425 | PASS | 9 | 16.22 | PASS | 13 | 9 | 11.69 | 1.39 | 1.39 | PTPN12 | 7q11.23 | protein tyrosine phosphatase, non- | Phosphatase |
| | | | | | | | | | | | | | | receptor type 12 | |
| PPP3CB2 | M29551_at | M29551 | PASS | 5 | 7.60 | PASS | 9 | 5 | 6.00 | 1.27 | 1.27 | | | | Phosphatase |
| PTPN4 | M66941_at | M68941 | PASS | 7 | 7.86 | PASS | 9 | 7 | 6.56 | 1.20 | 1.20 | PTPN4 | | protein tyrosine phosphatase, non- | Phosphatase |
| | | | | | | | | | | | | | | receptor type 4 (megakaryocyte) | |
| ACP1 | U25849_at | U25849 | PASS 6 | 9.83 | PASS | 13 | 6 | 8.69 | 1.13 | 1.13 | ACP1 | 2p25 | acid phosphatase 1, soluble | Phosphatase | |
| M60483_m | M60483_ma | M60483 | PASS | 8 | 6.50 | PASS | 12 | 8 | 5.75 | 1.13 | 1.13 | PPP2CA | 5q23-q31 | protein phosphatase 2 (formerly | Phosphatase |
| | | | | | | | | | | | | | | 2A), catalytic subunit, alpha | |
| | | | | | | | | | | | | | | isoform | |
| D11327_s_{—} | D11327_s_a | D11327 | PASS | 8 | 5 75 | PASS | 10 | 8 | 5.10 | 1.13 | 1.13 | PTPN7 | 1q32 1 | protein tyrosine phosphatase, non- | Phosphatase |
| | | | | | | | | | | | | | | receptor type 7 | |
| PPM1A | S87759_at | S87759 | PASS | 7 | 6.00 | PASS | 9 | 7 | 5.33 | 1 13 | 1.13 | PPM1B | | protein phosphatase 1B (formerly | Phosphatase |
| | | | | | | | | | | | | | | 2C), magnesium-dependent, beta | |
| | | | | | | | | | | | | | | isoform | |

TABLE 3
HuPBMC_RA_U95A-Kin-PhosP.xls

| Human RA PBMC data on U95 | | | | Human RA PBMC data on U95 | | | | | | | | | | | | |
|---|---|---|---|---|---|---|---|---|---|---|---|---|---|---|---|---|
| | | | | | | | | | Present | Present | Absent | | Fold | | | |
| | | | sum of | | Avg | Std | sum of | in RA | in RA, | in RA, | | Change | | | | |
| Affy | Affy | | Present | 4 of 6 | Freq | Dev | abs | 7 of 13 | and | Absent in | Present in | Avg Freq | RA/ | | Chromo- | Kinase or |
| Qualifier | Name | accession | Calls | present | RA | RA | dec | present | Normal | Normal | Normal | (Normal) | Normal | Name | some | Phosphatase |

| | | | | | | | | | | | | | | | | | | | | | | | | | | | | |
|---|---|---|---|---|---|---|---|---|---|---|---|---|---|---|---|---|---|---|---|---|---|---|---|---|---|---|---|---|
| Kinases | | | | | | | | | | | | | | | | | | | | | | | | | | | | |
| 446_at | CSNK1G2 | U89996 | 6 | Pass | 9.17 | 3 54 | 13 | Pass | TRUE | FALSE | FALSE | 4 69 | 1.95 | casein kinase 1, gamma 2; | 10p13 3 | Kinase | | | | | | | | | | | | |
| | | | | | | | | | | | | | | CSNK1G2 | | | | | | | | | | | | | | |
| 490_g_at | MUTYH | U63329 | 6 | Pass | 11.00 | 2.97 | 13 | Pass | TRUE | FALSE | FALSE | 5.62 | 1.96 | mutY (E coli ) homolog, | 1p34 3- | Kinase | | | | | | | | | | | | |
| | | | | | | | | | | | | | | MUTYH | p32 1 | | | | | | | | | | | | | |
| 41197_at | RAD23A | D21235 | 6 | Pass | 18 50 | 4 32 | 13 | Pass | TRUE | FALSE | FALSE | 9 38 | 1.97 | RAD23 (S cerevisiae) | 19p13 2 | Kinase | | | | | | | | | | | | |
| | | | | | | | | | | | | | | homolog A, RAD23A | | | | | | | | | | | | | | |
| 33300_at | CDC2L1 | AL031282 | 6 | Pass | 11 83 | 3.76 | 12 | Pass | TRUE | FALSE | FALSE | 5.92 | 2.00 | Cluster Inel AL031282: | | Kinase | | | | | | | | | | | | |
| | | | | | | | | | | | | | | Human DNA sequence | | | | | | | | | | | | | | |
| | | | | | | | | | | | | | | from clone 283E3 on | | | | | | | | | | | | | | |
| | | | | | | | | | | | | | | chromosome 1p36 21- | | | | | | | | | | | | | | |
| | | | | | | | | | | | | | | 36 33 Contains the | | | | | | | | | | | | | | |
| | | | | | | | | | | | | | | alternatively spliced gene | | | | | | | | | | | | | | |
| | | | | | | | | | | | | | | for Matrix | | | | | | | | | | | | | | |
| | | | | | | | | | | | | | | Metalloprotenase in the | | | | | | | | | | | | | | |
| | | | | | | | | | | | | | | Female Reproductive tract | | | | | | | | | | | | | | |
| | | | | | | | | | | | | | | MIF1, −2, and GSSs, | | | | | | | | | | | | | | |
| | | | | | | | | | | | | | | complete sequence. | | | | | | | | | | | | | | |
| 40742_at | HCK | M16591 | 6 | Pass | 52.50 | 31 65 | 13 | Pass | TRUE | FALSE | FALSE | 25 92 | 2.03 | hemopoiectic cell kinase, | 20q11- | Kinase | | | | | | | | | | | | |
| | | | | | | | | | | | | | | HCK | q12 | | | | | | | | | | | | | |
| 32799_at | C1ORF2 | AF023268 | 6 | Pass | 24.17 | 6 43 | 11 | Pass | TRUE | FALSE | FALSE | 11 91 | 2.03 | secretory cancer | | Kinase | | | | | | | | | | | | |
| | | | | | | | | | | | | | | membrane protein 3. | | | | | | | | | | | | | | |
| 1622_at | MAP2K3 | D87116 | 6 | Pass | 40.17 | 14.39 | 13 | Pass | TRUE | FALSE | FALSE | 19 77 | 2.03 | mitogen-activated protein | 17q11 2 | Kinase | | | | | | | | | | | | |
| | | | | | | | | | | | | | | kinase kinase 3; MAP2K3 | | | | | | | | | | | | | | |
| 146_at | PIK4CB | U81802 | 6 | Pass | 6 33 | 1.75 | 9 | Pass | TRUE | FALSE | FALSE | 3.11 | 2.04 | phosphatidylmosmol 4- | 1q21 | Kinase | | | | | | | | | | | | |
| | | | | | | | | | | | | | | kinase, catalytic, beta | | | | | | | | | | | | | | |
| | | | | | | | | | | | | | | polypeptide, PIK4CB | | | | | | | | | | | | | | |
| 1392_at | GPRK6 | L16862 | 4 | Pass | 28.25 | 5 50 | 7 | Pass | TRUE | FALSE | FALSE | 13.86 | 2.04 | G protein-coupled receptor | 5q35 | Kinase | | | | | | | | | | | | |
| | | | | | | | | | | | | | | kinase 6, GPRK6 | | | | | | | | | | | | | | |
| 33314_at | GCDH | U69141 | 6 | Pass | 6 17 | 1.17 | 11 | Pass | TRUE | FALSE | FALSE | 3.00 | 2.06 | glutaryl-Coenzyme A | 19p13 2 | Kinase | | | | | | | | | | | | |
| | | | | | | | | | | | | | | dehydrogenase, GCDH | | | | | | | | | | | | | | |
| 34808_at | KIAA0999 | AB023216 | 6 | Pass | 12 50 | 2.51 | 13 | Pass | TRUE | FALSE | FALSE | 6 08 | 2.06 | KIAA0999 protein, | | Kinase | | | | | | | | | | | | |
| | | | | | | | | | | | | | | KIAA0999 | | | | | | | | | | | | | | |
| 32046_at | PRKCD | D10495 | 6 | Pass | 26 93 | 9 93 | 13 | Pass | TRUE | FALSE | FALSE | 13 00 | 2.06 | protein kinase C, delta, | 3p | Kinase | | | | | | | | | | | | |
| | | | | | | | | | | | | | | PRKCD | | | | | | | | | | | | | | |
| 384_at | PSMB10 | X71874 | 6 | Pass | 50 00 | 22 63 | 13 | Pass | TRUE | FALSE | FALSE | 24 15 | 2.07 | protcasome (prosome, | 16q22 1 | Kinase | | | | | | | | | | | | |
| | | | | | | | | | | | | | | macropain) subunit, beta | | | | | | | | | | | | | | |
| | | | | | | | | | | | | | | type, 10 PSMB10 | | | | | | | | | | | | | | |
| 41249 at | UNK AL0 | AL031282 | 6 | Pass | 25 67 | 13 23 | 13 | Pass | TRUE | FALSE | FALSE | 12 31 | 2.09 | | | Kinase | | | | | | | | | | | | |
| 32716_at | DGKA | X62535 | 6 | Pass | 46 33 | 9.56 | 13 | Pass | TRUE | FALSE | FALSE | 22 08 | 2.10 | diacylglycerol kinase, | 12q13 3 | Kinase | | | | | | | | | | | | |
| | | | | | | | | | | | | | | alpha (80kD), DGKA | | | | | | | | | | | | | | |
| 1779 s at | PIM1 | M16750 | 6 | Pass | 42 00 | 9.61 | 13 | Pass | TRUE | FALSE | FALSE | 20 00 | 2.10 | pim-1 oncogene; PIM1 | 6p21.2 | Kinase | | | | | | | | | | | | |
| 31873_at | ARD1 | U52112 | 6 | Pass | 8.67 | 2 50 | 11 | Pass | TRUE | FALSE | FALSE | 4 09 | 2.12 | | | Kinase | | | | | | | | | | | | |
| 187_at | MAP4K2 | U07349 | 5 | Pass | 5 80 | 1 30 | 7 | Pass | TRUE | FALSE | FALSE | 2 71 | 2.14 | mitogen-activated protein | 11q13 | Kinase | | | | | | | | | | | | |
| | | | | | | | | | | | | | | kinase kinase kinase | | | | | | | | | | | | | | |
| | | | | | | | | | | | | | | kinase 2; MAP4K2 | | | | | | | | | | | | | | |
| 37910_at | HCFC1 | U52112 | 6 | Pass | 8 17 | 3.60 | 11 | Pass | TRUE | FALSE | FALSE | 3 82 | 2.14 | host cell factor C1 (VP16- | Xq28 | Kinase | | | | | | | | | | | | |
| | | | | | | | | | | | | | | accessory protein), | | | | | | | | | | | | | | |
| 1780_at | FGR | M19722 | 6 | Pass | 80 83 | 35.22 | 13 | Pass | TRUE | FALSE | FALSE | 37 77 | 2.14 | Gardner-Rasherd feline | 1p36 2- | Kinase | | | | | | | | | | | | |
| | | | | | | | | | | | | | | sarcoma viral (v-fgr) | | | | | | | | | | | | | | |
| | | | | | | | | | | | | | | oncogene homolog, FGR | | | | | | | | | | | | | | |
| 33281_at | KIAA0151 | D63485 | 6 | Pass | 14.33 | 1 63 | 13 | Pass | TRUE | FALSE | FALSE | 6.69 | 2.14 | IKK-related kinase | 1 | Kinase | | | | | | | | | | | | |
| | | | | | | | | | | | | | | epsilon, inducible IkappaB | | | | | | | | | | | | | | |
| | | | | | | | | | | | | | | kinase, IKKE | | | | | | | | | | | | | | |
| 39044_s_at | DGKD | D73409 | 6 | Pass | 31.67 | 7 76 | 13 | Pass | TRUE | FALSE | FALSE | 14 77 | 2.14 | diacylglycerol kinase, delta | | Kinase | | | | | | | | | | | | |
| | | | | | | | | | | | | | | (130kD), DGKD | | | | | | | | | | | | | | |
| 48420_at | STK10 | AB015718 | 6 | Pass | 35.17 | 8 70 | 13 | Pass | TRUE | FALSE | FALSE | 16 38 | 2.15 | serine/threonine kinase 10, | 5q35 1 | Kinase | | | | | | | | | | | | |
| | | | | | | | | | | | | | | STK10 | | | | | | | | | | | | | | |
| 40225_at | GAK | D88435 | 6 | Pass | 37 17 | 12 67 | 13 | Pass | TRUE | FALSE | FALSE | 17 31 | 2.15 | cyclin G associated kinase, | 4p16 | Kinase | | | | | | | | | | | | |
| | | | | | | | | | | | | | | GAK | | | | | | | | | | | | | | |
| 632_at | GSK3A | L40027 | 6 | Pass | 19 00 | 5.69 | 13 | Pass | TRUE | FALSE | FALSE | 8.77 | 2.17 | glycogcen synthase kinase 3 | | Kinase | | | | | | | | | | | | |
| | | | | | | | | | | | | | | alpha, GSK3A | | | | | | | | | | | | | | |
| 35796_at | PTK9L | Y17169 | 6 | Pass | 16.17 | 9.54 | 12 | Pass | TRUE | FALSE | FALSE | 7.42 | 2.18 | protein tyrosine kinase 9 | 3p21.1 | Kinase | | | | | | | | | | | | |
| | | | | | | | | | | | | | | like (A6-related protein); | | | | | | | | | | | | | | |
| | | | | | | | | | | | | | | PTK9L | | | | | | | | | | | | | | |
| 2075_s_at | MAP2K3 | L36719 | 6 | Pass | 17 67 | 4 23 | 12 | Pass | TRUE | FALSE | FALSE | 8 00 | 2.21 | mitogen-activated protein | 17q11 2 | Kinase | | | | | | | | | | | | |
| | | | | | | | | | | | | | | kinase kinase 3, MAP2K3 | | | | | | | | | | | | | | |
| 33301_g_at | CDC2L1 | AL031282 | 6 | Pass | 21 50 | 7.34 | 13 | Pass | TRUE | FALSE | FALSE | 9.69 | 2.22 | Cluster Incl AL031282 | | Kinase | | | | | | | | | | | | |
| | | | | | | | | | | | | | | Human DNA sequence | | | | | | | | | | | | | | |
| | | | | | | | | | | | | | | from clone 283b3 on | | | | | | | | | | | | | | |
| | | | | | | | | | | | | | | chromosome 1p36.21- | | | | | | | | | | | | | | |
| | | | | | | | | | | | | | | 36 33. Contains the | | | | | | | | | | | | | | |
| | | | | | | | | | | | | | | alternatively spliced gene | | | | | | | | | | | | | | |
| | | | | | | | | | | | | | | for Matrix | | | | | | | | | | | | | | |
| | | | | | | | | | | | | | | Metalloprotemase in the | | | | | | | | | | | | | | |
| | | | | | | | | | | | | | | Female Reproductive tract | | | | | | | | | | | | | | |
| | | | | | | | | | | | | | | MIFRL-2 | | | | | | | | | | | | | | |
| 1810_s_at | PRKCD | D10495 | 6 | Pass | 14.00 | 5.62 | 11 | Pass | TRUE | FALSE | FALSE | 6 27 | 2.23 | protein kinase C, delta, | 3p | Kinase | | | | | | | | | | | | |
| | | | | | | | | | | | | | | PRKCD | | | | | | | | | | | | | | |
| 36949 at | CSNK1D | U29171 | 6 | Pass | 45 00 | 11.93 | 13 | Pass | TRUE | FALSE | FALSE | 20 08 | 2.24 | casein kinase 1, delta, | 17q25 | Kinase | | | | | | | | | | | | |
| | | | | | | | | | | | | | | CSNK1D | | | | | | | | | | | | | | |
| 1707_g_at | ARAF1 | U01337 | 6 | Pass | 31.00 | 6.75 | 11 | Pass | TRUE | FALSE | FALSE | 13.82 | 2.24 | v-ref murine sarcoma 3611 | Xp11 4- | Kinase | | | | | | | | | | | | |
| | | | | | | | | | | | | | | viral oncogene homolog 1, | p11 2 | | | | | | | | | | | | | |
| | | | | | | | | | | | | | | ARAF1 Ser/Thr protein | | | | | | | | | | | | | | |
| | | | | | | | | | | | | | | kinase | | | | | | | | | | | | | | |
| 38617_at | LIMK2 | D45906 | 5 | Pass | 9.00 | 2.55 | 11 | Pass | TRUE | FALSE | FALSE | 4.00 | 2.25 | LIM domain kinase 2, | 22qt2 2 | Kinase | | | | | | | | | | | | |
| | | | | | | | | | | | | | | LIMK2 | | | | | | | | | | | | | | |
| 35299_at | MKNK1 | AB000409 | 6 | Pass | 8 67 | 2 73 | 12 | Pass | TRUE | FALSE | FALSE | 3.83 | 2.26 | MAP kinase-interacting | | Kinase | | | | | | | | | | | | |
| | | | | | | | | | | | | | | serine/threonine kinase 1; | | | | | | | | | | | | | | |
| | | | | | | | | | | | | | | MKNK1 | | | | | | | | | | | | | | |
| 34291_at | FARSL | U07424 | 6 | Pass | 11.00 | 3.35 | 13 | Pass | TRUE | FALSE | FALSE | 4.77 | 2.31 | phenylalanine-tRNA | 19p13 2 | Kinase | | | | | | | | | | | | |
| | | | | | | | | | | | | | | synthetase-like; FARLSL | | | | | | | | | | | | | | |
| 1498_at | ZAP70 | L05148 | 6 | Pass | 35 67 | 9 71 | 13 | Pass | TRUE | FALSE | FALSE | 15.38 | 2.32 | zeta-chain (TCR) | 2q12 | Kinase | | | | | | | | | | | | |
| | | | | | | | | | | | | | | associated protein kinase | | | | | | | | | | | | | | |
| | | | | | | | | | | | | | | (70 kD), ZAP70 | | | | | | | | | | | | | | |
| 1706_at | ARAF1 | U01337 | 6 | Pass | 25.17 | 6.43 | 13 | Pass | TRUE | FALSE | FALSE | 10 69 | 2.35 | v-ref murine sarcoma 3611 | Xp11.4- | Kinase | | | | | | | | | | | | |
| | | | | | | | | | | | | | | viral oncogene homolog 1, | p11 2 | | | | | | | | | | | | | |
| | | | | | | | | | | | | | | ARAF1 | | | | | | | | | | | | | | |
| 1127_at | RPS6KA1 | L07597 | 6 | Pass | 33 67 | 13 82 | 13 | Pass | TRUE | FALSE | FALSE | 14 15 | 2.38 | ribosomal protein S6 | 3 | Kinase | | | | | | | | | | | | |
| | | | | | | | | | | | | | | kinase, 90kD, polypeptide | | | | | | | | | | | | | | |
| | | | | | | | | | | | | | | 1:RPS6KA1 | | | | | | | | | | | | | | |
| 36179_at | MAPKAPH | U12779 | 6 | Pass | 36.67 | 8.19 | 13 | Pass | TRUE | FALSE | FALSE | 15.31 | 2.40 | mitogen-activated protein | | Kinase | | | | | | | | | | | | |
| | | | | | | | | | | | | | | kinase-activated protein | | | | | | | | | | | | | | |
| | | | | | | | | | | | | | | kinase 2; MAPKAPK2 | | | | | | | | | | | | | | |
| 1652 at | PIM2 | U77735 | 6 | Pass | 17 50 | 7 82 | 11 | Pass | TRUE | FALSE | FALSE | 7.27 | 2.41 | pim-2 oncogene, PIM2 | X | Kinase | | | | | | | | | | | | |
| 883_s at | PIM1 | M54915 | 6 | Pass | 59.00 | 8 44 | 13 | Pass | TRUE | FALSE | FALSE | 24.46 | 2.41 | pim-1 oncogene, PIM1 | 6p21.2 | Kinase | | | | | | | | | | | | |
| 33804_at | PTK2B | U43522 | 6 | Pass | 16.67 | 7.63 | 8 | Pass | TRUE | FALSE | FALSE | 6 88 | 2.42 | protein tyrosine kinase 2 | 8p21.1 | Kinase | | | | | | | | | | | | |
| | | | | | | | | | | | | | | beta, PTK2B | | | | | | | | | | | | | | |
| 493_at | CSNK1D | U29171 6 | Pass | 17.17 | 6 43 | 13 | Pass | TRUE | FALSE | FALSE | 7 08 | 2.43 | casein kinase 1, delta, | 17q25 | Kinase | | | | | | | | | | | | | |
| | | | | | | | | | | | | | | CSNK1D | | | | | | | | | | | | | | |
| 1398_g_at | MAP3K11 | L32976 | 6 | Pass | 27.83 | 17.62 | 13 | Pass | TRUE | FALSE | FALSE | 11.46 | 2.43 | mitogen-activated protein | 11q13 1- | Kinase | | | | | | | | | | | | |
| | | | | | | | | | | | | | | kinase kinase kinase 11, | q13 3 | | | | | | | | | | | | | |
| | | | | | | | | | | | | | | MAP3K11 | | | | | | | | | | | | | | |
| 33903_at | DAPK3 | AB007144 | 5 | Pass | 13 00 | 5.61 | 9 | Pass | TRUE | FALSE | FALSE 5 22 | 2.49 | death-associated protein | 19p13 3 | Kinase | | | | | | | | | | | | | |
| | | | | | | | | | | | | | | kinase 3; DAPK3 | | | | | | | | | | | | | | |
| 1134_at | ACK | L13738 | 6 | Pass | 25.00 | 7.07 | 12 | Pass | TRUE | FALSE | FALSE | 10.00 | 2.50 | activated p2lcdc42Hs | 3 | Kinase | | | | | | | | | | | | |
| | | | | | | | | | | | | | | kinase, ACK1 | | | | | | | | | | | | | | |
| 33223_at | KIAA0561 | AB011133 | 6 | Pass | 16.17 | 7 17 | 13 | Pass | TRUE | FALSE | FALSE | 6 46 | 2.50 | KIAA0561 protein, | | Kinase | | | | | | | | | | | | |
| | | | | | | | | | | | | | | KIAA0561 | | | | | | | | | | | | | | |
| 32004_s at | UNK W32 | W32483 | 6 | Pass | 10.83 | 5 04 | 13 | Pass | TRUE | FALSE | FALSE | 4.31 | 2.51 | | | Kinase | | | | | | | | | | | | |
| 34679_at | BCR | X02596 | 5 | pass | 17.00 | 2.00 | 12 | Pass | TRUE | FALSE | FALSE | 6.67 | 2.55 | brcakpoint cluster region, | 22q11 23 | Kinase | | | | | | | | | | | | |
| | | | | | | | | | | | | | | BCR | | | | | | | | | | | | | | |
| 35365_at | ILK | U40282 | 6 | Pass | 30 17 | 5.98 | 13 | Pass | TRUE | FALSE | FALSE | 11 62 | 2.60 | integrin-linked kinase, ILK | 11p15 5- | Kinase | | | | | | | | | | | | |
| | | | | | | | | | | | | | | | p15 4 | | | | | | | | | | | | | |
| 38269_at | DKFZP586 | AL050147 | 6 | Pass | 44 67 | 9.63 | 13 | Pass | TRUE | FALSE | FALSE | 16.92 | 2.64 | DKFZP586E0820 protein; | 19 | Kinase | | | | | | | | | | | | |
| | | | | | | | | | | | | | | KDFZP586E0820 | | | | | | | | | | | | | | |
| 1768_s_at | CSK | X59932 | 6 | Pass | 75 50 | 29 19 | 13 | Pass | TRUE | FALSE | FALSE | 27.77 | 2.72 | c-src tyrosine kinase, CSK | 15q23- | Kinase | | | | | | | | | | | | |
| | | | | | | | | | | | | | | | q25 | | | | | | | | | | | | | |
| 38003_2_at | DGKZ | U94905 | 6 | Pass | 29 17 | 12 50 | 13 | Pass | TRUE | FALSE | FALSE | 10.23 | 2.85 | diacylglycerol kinase, zeta | | Kinase | | | | | | | | | | | | |
| | | | | | | | | | | | | | | (104kD), DGKZ | | | | | | | | | | | | | | |
| 138_at | MAP4K1 | U66464 | 6 | Pass | 16 33 | 4 13 | 13 | Pass | TRUE | FALSE | FALSE | 5 69 | 2.87 | mitogen-activated protein | 19q13 1- | Kinase | | | | | | | | | | | | |
| | | | | | | | | | | | | | | kinase kinase kinase | | | | | | | | | | | | | | |
| | | | | | | | | | | | | | | kinase 1, MAP4K1 | | | | | | | | | | | | | | |
| 993 at | TYK2 | X54637 | 6 | Pass | 15 50 | 5 24 | 12 | Pass | TRUE | FALSE | FALSE | 5.25 | 2.95 | tyrosine kinase 2; TYK2 | 19p13.2 | Kinase | | | | | | | | | | | | |
| 40235_at | ACK | L13738 | 6 | Pass | 13.33 | 5.05 | 10 | Pass | TRUE | FALSE | FALSE | 3.70 | 3.60 | activated p21cdc4211s | 3 | Kinase | | | | | | | | | | | | |
| | | | | | | | | | | | | | | kinase, ACK1 | | | | | | | | | | | | | | |
| Phosphatases | | | | | | | | | | | | | | | | | | | | | | | | | | | | |
| 37384_at | KIAA0015 | D13640 | 6 | Pass | 18 00 | 5 40 | 10 | Pass | TRUE | FALSE | FALSE | 8 80 | 2.05 | KIAA0015 gene product, | 22q11 22 | Phosphatase | | | | | | | | | | | | |
| | | | | | | | | | | | | | | KIAA0015 | | | | | | | | | | | | | | |
| 172_at | INPP5D | U57650 | 6 | Pass | 33 00 | 10 08 | 13 | Pass | TRUE | FALSE | FALSE | 16 08 | 2.05 | inositol polyphosphate-5- | 2q36-q37 | Phosphatase | | | | | | | | | | | | |
| | | | | | | | | | | | | | | phosphatase, 145kD, | | | | | | | | | | | | | | |
| | | | | | | | | | | | | | | INPP5D | | | | | | | | | | | | | | |
| 41162_at | PPM1G | Y13936 | 6 | Pass | 20.00 | 4 05 | 13 | Pass | TRUE | FALSE | FALSE | 9.23 | 2.17 | protein phosphatase 1G | | Posphatase | | | | | | | | | | | | |
| | | | | | | | | | | | | | | (formerly 2C), magnesium- | | | | | | | | | | | | | | |
| | | | | | | | | | | | | | | dependent, gamma | | | | | | | | | | | | | | |
| | | | | | | | | | | | | | | isoform, PPM1G | | | | | | | | | | | | | | |
| 382_at | PPP4C | X70218 | 6 | Pass | 16 83 | 7.36 | 13 | Pass | TRUE | FALSE | FALSE | 7.46 | 2.26 | protein phosphatase 4 | 16p12- | Phosphatase | | | | | | | | | | | | |
| | | | | | | | | | | | | | | (formerly X), catalytic | 16p11 | | | | | | | | | | | | | |
| | | | | | | | | | | | | | | subunit, PPP4C | | | | | | | | | | | | | | |
| 210_at | PLCB2 | M95678 | 6 | Pass | 49 17 | 20.53 | 12 | Pass | TRUE | FALSE | FALSE | 20 58 | 2.39 | phospholipase C, beta 2; | 15q15 | Phosphatase | | | | | | | | | | | | |
| | | | | | | | | | | | | | | PLCB2 | | | | | | | | | | | | | | |
| 41225_at | UNK_AL0 | AL049417 | 6 | Pass | 9 17 | 4 88 | 13 | Pass | TRUE | FALSE | FALSE | 3.77 | 2.43 | dual specificity | 17q21 | Pbosphatase | | | | | | | | | | | | |
| | | | | | | | | | | | | | | phosphatase 3 (vaccmia | | | | | | | | | | | | | | |
| | | | | | | | | | | | | | | virus phosphatase VH1- | | | | | | | | | | | | | | |
| | | | | | | | | | | | | | | related), DUSP3 | | | | | | | | | | | | | | |
| 794_at | PTPN6 | X62055 | 6 | Pass | 28.50 | 11.93 | 13 | Pass | TRUE | FALSE | FALSE | 10.69 | 2.67 | protein tyrosine | 12p13 | Phosphatase | | | | | | | | | | | | |
| | | | | | | | | | | | | | | phosphatase, non-receptor | | | | | | | | | | | | | | |
| | | | | | | | | | | | | | | type 6; PTPN6 | | | | | | | | | | | | | | |
| 1005_at | DUSP1 | X68277 | 6 | Pass | 21 17 | 21 76 | 13 | Pass | TRUE | FALSE | FALSE | 6 46 | 3.28 | dual specificity | 5q34 | Phosphatase | | | | | | | | | | | | |
| | | | | | | | | | | | | | | phosphatase 1, DUSP1 | | | | | | | | | | | | | | |
| 37864_s_at | IGHG3 | Y14737 | 6 | Pass | 93.83 | 76.26 | 13 | Pass | TRUE | FALSE | FALSE | 8 00 | 11.73 | immunoglobulin heavy | 14q32 33 | | | | | | | | | | | | | |
| | | | | | | | | | | | | | | constant gamma 3 (G3m | | | | | | | | | | | | | | |
| | | | | | | | | | | | | | | marker), IGHG3 | | | | | | | | | | | | | | |
| 36482_s_at | ATP2A3 | Y15724 | 6 | Pass | 17 00 | 5 33 | 12 | Pass | TRUE | FALSE | FALSE | 3 25 | 5.23 | ATPasc, Ca++ | 17p13 3 | | | | | | | | | | | | | |
| | | | | | | | | | | | | | | transporting, ubiquitous, | | | | | | | | | | | | | | |
| | | | | | | | | | | | | | | ATP2A3 | | | | | | | | | | | | | | |
| 40644_g_at | ITGA2B | M34480 | 6 | Pass | 37 83 | 9.95 | 11 | Pass | TRUE | FALSE | FALSE | 7.45 | 5.08 | integrin, alpha 26 (platelet | 17q21 32 | | | | | | | | | | | | | |
| | | | | | | | | | | | | | | glycoprotein IIb of IIb/IIIa | | | | | | | | | | | | | | |
| | | | | | | | | | | | | | | complex, antigen CD41B), | | | | | | | | | | | | | | |
| | | | | | | | | | | | | | | ITGA2B | | | | | | | | | | | | | | |
| 32749_s_at | FLNA | AL050396 | 6 | Pass | 209.83 | 58 51 | 13 | Pass | TRUE | FALSE | FALSE | 41 46 | 5.06 | filamn A. alpha (actni- | Xq28 | | | | | | | | | | | | | |
| | | | | | | | | | | | | | | binding protein-280), | | | | | | | | | | | | | | |
| | | | | | | | | | | | | | | FLNA | | | | | | | | | | | | | | |
| 33501_r_{—l at} | IGHA1 | S71043 | 6 | Pass | 138 50 | 88.39 | 13 | Pass | TRUE | FALSE | FALSE | 28.92 | 4.79 | immunoglobulin heavy | 14q32 33 | | | | | | | | | | | | | |
| | | | | | | | | | | | | | | constant alpha 1; IGHA1 | | | | | | | | | | | | | | |
| 33822_at | NUMA1 | Z11584 | 6 | Pass | 11.50 | 6 35 | 12 | Pass | TRUE | FALSE | FALSE | 2.42 | 4.76 | nuclear mitotic apparatus | 11q13 | | | | | | | | | | | | | |
| | | | | | | | | | | | | | | protein 1, NUMA1 | | | | | | | | | | | | | | |
| 38487 at | KIAA0246 | D87433 | 6 | Pass | 35.50 | 24 04 | 8 | Pass | TRUE | FALSE | FALSE | 7 63 | 4.66 | Stabilm-1 | | | | | | | | | | | | | | |
| 1268_at | UBE1 | M58028 | 6 | Pass | 47.50 | 14 10 | 13 | Pass | TRUE | FALSE | FALSE | 10.54 | 4.51 | ubiquitan-activating | Xp11 23 | | | | | | | | | | | | | |
| | | | | | | | | | | | | | | enzyme E1 (A1S9T and | | | | | | | | | | | | | | |
| | | | | | | | | | | | | | | BN75 temperature | | | | | | | | | | | | | | |
| | | | | | | | | | | | | | | sensitivity | | | | | | | | | | | | | | |
| 37467_at | IGHD | K02882 | 6 | Pass | 25 17 | 37.82 | 10 | Pass | TRUE | FALSE | FALSE | 5.60 | 4.49 | immunoglobulin heavy | 14q32 33 | | | | | | | | | | | | | |
| | | | | | | | | | | | | | | constant delta, IGHD | | | | | | | | | | | | | | |
| 32378_at | PKM2 | M26252 | 6 | Pass | 96.17 | 29.53 | 13 | Pass | TRUE | FALSE | FALSE | 21.77 | 4.42 | pyruvate kinase, muscle; | 15q22 | | | | | | | | | | | | | |
| | | | | | | | | | | | | | | PMK2 | | | | | | | | | | | | | | |
| 39049_at | NOTCH4 | 6 | Pass | 47 33 | 13.25 | 13 | Pass | TRUE | FALSE | FALSE | 11.31 | 4.19 | Notch (Drosophula) | 6p21.3 | | | | | | | | | | | | | | |
| | | | | | | | | | | | | | | homolog 4, NOTCH4 | | | | | | | | | | | | | | |
| 33499_s_at | IGHA1 | AF067420 | 6 | Pass | 123 33 | 74 06 | 13 | Pass | TRUE | FALSE | FALSE | 29 62 | 4.16 | immunoglobulin heavy | 14q32 33 | | | | | | | | | | | | | |
| | | | | | | | | | | | | | | constant alpha 1, IGHA1 | | | | | | | | | | | | | | |
| 36028_at | TCIRG1 | U45285 | 6 | Pass | 33.17 | 17.12 | 13 | Pass | TRUE | FALSE | FALSE | 8.00 | 4.15 | T-cell, immune regulator | 11q13 4- | | | | | | | | | | | | | |
| | | | | | | | | | | | | | | 1, TCIRG1 | q13 5 | | | | | | | | | | | | | |
| 32070_at | PTPRCAP | X7267 | 6 | Pass | 119.83 | 16 22 | 13 | Pass | TRUE | FALSE | FALSE | 28 92 | 4.14 | protein tyrosine | 11q13 3 | | | | | | | | | | | | | |
| | | | | | | | | | | | | | | phoshatase, receptor type, | | | | | | | | | | | | | | |
| | | | | | | | | | | | | | | c polypeptide-associated | | | | | | | | | | | | | | |
| | | | | | | | | | | | | | | protein, PTPRCAP | | | | | | | | | | | | | | |
| 32588_s_at | BRF2 | X78992 | 6 | Pass | 105.50 | 33.35 | 13 | Pass | TRUE | FALSE | FALSE | 25 62 | 4.12 | butyrate response factor 2 | | | | | | | | | | | | | | |
| | | | | | | | | | | | | | | (EGF-response factor 2), | | | | | | | | | | | | | | |
| | | | | | | | | | | | | | | BRF2 | | | | | | | | | | | | | | |
| 37014_at | MX1 | M33882 | 6 | Pass | 27 50 | 22.98 | 13 | Pass | TRUE | FALSE | FALSE | 6 69 | 4.11 | myxovus (influenza) | 21q22 3 | | | | | | | | | | | | | |
| | | | | | | | | | | | | | | resistance 1, homolog of | | | | | | | | | | | | | | |
| | | | | | | | | | | | | | | murine (interferon- | | | | | | | | | | | | | | |
| | | | | | | | | | | | | | | inducible protein p78). | | | | | | | | | | | | | | |
| 32623_at | GABBR1 | AJ225028 | 6 | Pass | 18 17 | 4.17 | 7 | Pass | TRUE | FALSE | FALSE | 4 43 | 4.10 | gamma-aminobutyric acid | 6p21.3 | | | | | | | | | | | | | |
| | | | | | | | | | | | | | | (GABA) B receptor, 1, | | | | | | | | | | | | | | |
| | | | | | | | | | | | | | | GABBR1 | | | | | | | | | | | | | | |
| 596_s_at | CSF3R | M59820 | 6 | Pass | 40 67 | 20.97 | 12 | Pass | TRUE | FALSE | FALSE | 9.92 | 4.10 | colony stimulating factor 3 | 1p35- | | | | | | | | | | | | | |
| | | | | | | | | | | | | | | receptor (granulocyte), p34 3 | | | | | | | | | | | | | | |
| | | | | | | | | | | | | | | CSF3R | | | | | | | | | | | | | | |
| 1915_s_at | FOS | V01512 | 6 | Pass | 63 50 | 46 86 | 13 | Pass | TRUE | FALSE | FALSE | 15 54 | 4.09 | v-fos FBJ murine | 14q24 3 | | | | | | | | | | | | | |
| | | | | | | | | | | | | | | osteosarcoma viral | | | | | | | | | | | | | | |
| | | | | | | | | | | | | | | oncogene homolog, FOS | | | | | | | | | | | | | | |
| 36412_s_at | IRF7 | U53831 | 6 | Pass | 17 00 | 10.66 | 12 | Pass | TRUE | FALSE | FALSE 4.17 | 4.08 | interferon regulatory factor | 11 | | | | | | | | | | | | | | |
| | | | | | | | | | | | | | | 7, IRF7 | | | | | | | | | | | | | | |
| 36138_at | CAPN4 | X04106 | 6 | Pass | 57.67 | 25 33 | 13 | Pass | TRUE | FALSE | FALSE | 14 38 | 4.01 | calpain, small polypeptide; | 19 | | | | | | | | | | | | | |
| | | | | | | | | | | | | | | CAPN4 | | | | | | | | | | | | | | |
| 36879_at | ECGF1 | M63193 | 6 | Pass | 89 00 | 78 82 | 12 | Pass | TRUE | FALSE | FALSE | 22 33 | 3.99 | endothelial cell growth | 22q13 33 | | | | | | | | | | | | | |
| | | | | | | | | | | | | | | factor, 1 (platelet-derived), | | | | | | | | | | | | | | |
| | | | | | | | | | | | | | | EGGF1 | | | | | | | | | | | | | | |
| 1916_s_at | FOS | V01512 | 6 | Pass | 46.17 | 29.96 | 13 | Pass | TRUE | FALSE | FALSE | 12.00 | 3.85 | v-fos murine | 14q4 3 | | | | | | | | | | | | | |
| | | | | | | | | | | | | | | osteosarcoma viral | | | | | | | | | | | | | | |
| | | | | | | | | | | | | | | oncogene homolog, FOS | | | | | | | | | | | | | | |
| 33273_f_at | IGL@ | X57809 | 6 | Pass | 180 83 | 155.56 | 13 | Pass | TRUE | FALSE | FALSE | 47 38 | 3.82 | immunoglobulin lambda | 22q11 1- | | | | | | | | | | | | | |
| | | | | | | | | | | | | | | locus, IGL,⅝ | q11 2 | | | | | | | | | | | | | |
| 34874_at | NTE | AJ004832 | 6 | Pass | 23 00 | 12.44 | 12 | Pass | TRUE | FALSE | FALSE | 6 08 | 3.78 | neutropathy target esterase, | 19p | | | | | | | | | | | | | |
| | | | | | | | | | | | | | | NTE | | | | | | | | | | | | | | |
| 41827 f at | UNK AI93 | AI932613 | 6 | Pass | 58.67 | 49.98 | 13 | Pass | TRUE | FALSE | FALSE | 15.69 | 3.74 | SMA3, SMA3 | 5q13 | | | | | | | | | | | | | |
| 38647_at | COPE | AJ131182 | 6 | Pass | 44 83 | 17.38 | 13 | Pass | TRUE | FALSE | FALSE | 12.00 | 3.74 | coatomer protein complex, | | | | | | | | | | | | | | |
| | | | | | | | | | | | | | | subunit epsilon, COPE | | | | | | | | | | | | | | |
| 40164_at | ARHGDIA | X69550 | 6 | Pass | 42 17 | 14.66 | 13 | Pass | TRUE | FALSE | FALSE | 11 31 | 3.73 | Rho GDP dissociation | 17q25 3 | | | | | | | | | | | | | |
| | | | | | | | | | | | | | | inhibitor (GDI) alpha, | | | | | | | | | | | | | | |
| | | | | | | | | | | | | | | ARHGDIA | | | | | | | | | | | | | | |
| 33500_t_at | IGHA1 | S71043 | 6 | Pass | 109.33 | 64 26 | 13 | Pass | TRUE | FALSE | FALSE | 30 00 | 3.64 | immunoglobulin heavy | 14q32.33 | | | | | | | | | | | | | |
| | | | | | | | | | | | | | | constant alpha 1, IGHA1 | | | | | | | | | | | | | | |
| 40718_at | CTSW | AF013611 | 6 | Pass | 30 50 | 19.00 | 10 | Pass | TRUE | FALSE | FALSE | 8.40 | 3.63 | cathepsin W | 11q13 1 | | | | | | | | | | | | | |
| | | | | | | | | | | | | | | (lymphopam), CTSW | | | | | | | | | | | | | | |
| 33143_s_at | SLC16A3 | U81800 | 6 | Pass | 43.83 | 23.80 | 13 | Pass | TRUE | FALSE | FALSE | 12 15 | 3.61 | solute carrier family 16 | 22q12 3- | | | | | | | | | | | | | |
| | | | | | | | | | | | | | | (monocarboxylic acid | q13 2 | | | | | | | | | | | | | |
| | | | | | | | | | | | | | | transporters), member 3. | | | | | | | | | | | | | | |
| | | | | | | | | | | | | | | SLC16A3 | | | | | | | | | | | | | | |
| 239_at | CTSD | M63138 | 6 | Pass | 76.50 | 38.12 | 13 | Pass | TRUE | FALSE | FALSE | 21.31 | 3.59 | cathepsin D (lysosomal | 11p15.5 | | | | | | | | | | | | | |
| | | | | | | | | | | | | | | aspartyl protease), CTSD | | | | | | | | | | | | | | |
| 33816_at | UNK AF0 | AF020267 | 6 | Pass | 17 33 | 8.45 | 12 | Pass | TRUE | FALSE | FALSE | 4 92 | 3.53 | myosin 1XB, MYO9B | 19p13 1 | | | | | | | | | | | | | |
| 33283 at | ARRB2 | AF106941 | 6 | Pass | 84.00 | 41.95 | 13 | Pass | TRUE | FALSE | FALSE | 24 00 | 3.50 | arrestin, beta 2, ARRB2 | 17p13 | | | | | | | | | | | | | |
| 3275O_r_at | FLNA | X53416 | 6 | Pass | 23 00 | 3 95 | 10 | Pass | TRUE | FALSE | FALSE | 6.60 | 3.48 | filimin A, alpha (actin- | Xq28 | | | | | | | | | | | | | |
| | | | | | | | | | | | | | | binding protein-280), | | | | | | | | | | | | | | |
| | | | | | | | | | | | | | | FLNA | | | | | | | | | | | | | | |
| 31874_at | GAR22 | Y07846 | 6 | Pass | 15.67 | 5.82 | 12 | Pass | TRUE | FALSE | FALSE | 4.50 | 3.48 | GAS2-related on | 22q12.2 | | | | | | | | | | | | | |
| | | | | | | | | | | | | | | chromosome 22; GAR22 | | | | | | | | | | | | | | |
| 39997_at | PFC | AF005664 | 6 | Pass | 57 00 | 28.64 | 13 | Pass | TRUE | FALSE | FALSE | 16 69 | 3.41 | properdin P factor, | Xp11 3- | | | | | | | | | | | | | |
| | | | | | | | | | | | | | | complement, PTC | p11 23 | | | | | | | | | | | | | |
| 410145_at | SECTM1 | U77643 | 6 | Pass | 29 67 | 22.56 | 13 | Pass | TRUE | FALSE | FALSE | 8 69 | 3.41 | secreted and | 17q25 | | | | | | | | | | | | | |
| | | | | | | | | | | | | | | transmembrane 1, | | | | | | | | | | | | | | |
| 34412_s_at | GP1BB | U59632 | 6 | Pass | 85 50 | 35.30 | 13 | Pass | TRUE | FALSE | FALSE | 25.08 | 3.41 | glycoprotein lb (platelet), | 22q11 21 | | | | | | | | | | | | | |
| | | | | | | | | | | | | | | beta polypeptide, GP1BB | | | | | | | | | | | | | | |
| 33274_f_at | IGL@ | M18645 | 6 | Pass | 162 50 | 133 56 | 13 | Pass | TRUE | FALSE | FALSE | 48.15 | 3.37 | immunoglobulin lambda | 22q11.1- | | | | | | | | | | | | | |
| | | | | | | | | | | | | | | locus, IGL@ | q11 2 | | | | | | | | | | | | | |
| 35170_at | MAN2C1 | AF044414 | 6 | Pass | 21 50 | 5 43 | 10 | Pass | TRUE | FALSE | FALSE | 6 40 | 3.36 | mammosidasc, alpha, class | 15q11- | | | | | | | | | | | | | |
| | | | | | | | | | | | | | | 2C, member 1, MAN2C1 | | | | | | | | | | | | | | |
| 41168_at | TAPBP | AF029750 | 6 | Pass | 112 50 | 29.72 | 13 | Pass | TRUE | FALSE | FALSE | 34 00 | 3.31 | TAP binding protein | 6p21.3 | | | | | | | | | | | | | |
| | | | | | | | | | | | | | | (tapasm); TAPBP | | | | | | | | | | | | | | |
| 37192_at | EPB49 | U28389 | 6 | Pass | 56 67 | 18 65 | 12 | Pass | TRUE | FALSE | FALSE | 17 17 | 3.30 | erythrocyte membrane | 8p21 1 | | | | | | | | | | | | | |
| | | | | | | | | | | | | | | protein band 4 9 | | | | | | | | | | | | | | |
| | | | | | | | | | | | | | | (dematin), EPB49 | | | | | | | | | | | | | | |
| 38138_at | S100A11 | D38583 | 6 | Pass | 74 83 | 42 88 | 13 | Pass | TRUE | FALSE | FALSE | 22.77 | 3.29 | S100 calcium-binding | 1q21 | | | | | | | | | | | | | |
| | | | | | | | | | | | | | | protein A11 (calgzim), | | | | | | | | | | | | | | |
| | | | | | | | | | | | | | | S100A11 | | | | | | | | | | | | | | |
| 41446_f_at | RNAHP | H68340 | 6 | Pass | 25 67 | 13.92 | 13 | Pass | TRUE | FALSE | FALSE | 7 85 | 3.27 | RNA helicase-related | 17 | | | | | | | | | | | | | |
| | | | | | | | | | | | | | | protein, RNAHP | | | | | | | | | | | | | | |
| 40643_at | ITGA2B | M34480 | 6 | Pass | 101 33 | 18 45 | 13 | Pass | TRUE | FALSE | FALSE | 31 00 | 3.27 | integrin, alpha 2b (platelet | 17q21.32 | | | | | | | | | | | | | |
| | | | | | | | | | | | | | | glycoprotein IIb of IIb/IIIa | | | | | | | | | | | | | | |
| | | | | | | | | | | | | | | complex, antigen CD41B); | | | | | | | | | | | | | | |
| | | | | | | | | | | | | | | ITGA2B | | | | | | | | | | | | | | |
| 37966_{—l at} | UNK_AA1 | AA187563 | 6 | Pass | 10 17 | 3 43 | 8 | Pass | TRUE | FALSE | FALSE | 3 13 | 3.25 | CGI-56 protein, CGI-56 | 22q13 2- | | | | | | | | | | | | | |
| | | | | | | | | | | | | | | | q13 33 | | | | | | | | | | | | | |
| 33425_at | TIF1B | X97548 | 6 | Pass | 30.67 | 7 53 | 13 | Pass | TRUE | FALSE | FALSE | 9.46 | 3.24 | KRAB-associated protein | 5 | | | | | | | | | | | | | |
| | | | | | | | | | | | | | | 1, TIF1B | | | | | | | | | | | | | | |
| 36493_at | LSP1 | M33552 | 6 | Pass | 49 17 | 23 56 | 13 | Pass | TRUE | FALSE | FALSE | 15 31 | 3.21 | lymphocyte-specific | 11p15 5 | | | | | | | | | | | | | |
| | | | | | | | | | | | | | | protein 1, LSP1 | | | | | | | | | | | | | | |
| 34223_at | CSF3R | M59818 | 6 | Pass | 34 33 | 16.75 | 13 | Pass | TRUE | FALSE | FALSE | 10 69 | 3.21 | colony stimulating factor 3 | 1p35- | | | | | | | | | | | | | |
| | | | | | | | | | | | | | | receptor (granulocyte), | p34 3 | | | | | | | | | | | | | |
| | | | | | | | | | | | | | | CSF3R | | | | | | | | | | | | | | |
| 34780 at | PLXNB2 | AB002313 | 6 | Pass | 43 67 | 21.77 | 13 | Pass | TRUE | FALSE | FALSE | 13 62 | 3.21 | plexin B2, PLXNB2 | 22q13 33 | | | | | | | | | | | | | |
| 39649_at | ARHGAP4 | X78817 | 6 | Pass | 53.00 | 19.42 | 13 | Pass | TRUE | FALSE | FALSE | 16.54 | 3.20 | Rho GTPase activating | Xq28 | | | | | | | | | | | | | |
| | | | | | | | | | | | | | | protein 4, ARHGAP4 | | | | | | | | | | | | | | |
| 35786_at | KIAA0476 | AB007945 | 6 | Pass | 28 33 | 9.29 | 13 | Pass | TRUE | FALSE | FALSE | 8.85 | 3.20 | KIAA0476 gene product, | 1 | | | | | | | | | | | | | |
| | | | | | | | | | | | | | | KIAA0476 | | | | | | | | | | | | | | |
| 39424_at | TNFRSF14 | U70321 | 6 | Pass | 26 50 | 9.81 | 13 | Pass | TRUE | FALSE | FALSE | 8 31 | 3.19 | tumor necrosis factor | 1p136 3- | | | | | | | | | | | | | |
| | | | | | | | | | | | | | | receptor superfamily, | p36 2 | | | | | | | | | | | | | |
| | | | | | | | | | | | | | | member 14 (herpesvirus | | | | | | | | | | | | | | |
| | | | | | | | | | | | | | | entry moderator), | | | | | | | | | | | | | | |
| | | | | | | | | | | | | | | TNFRSF14 | | | | | | | | | | | | | | |
| 1353_g_at | IL8RA | U11870 | 4 | Pass | 12 75 | 2.22 | 8 | Pass | TRUE | FALSE | FALSE | 4.00 | 3.19 | interleukin 8 receptor, | 2q35 | | | | | | | | | | | | | |
| | | | | | | | | | | | | | | alpha, IL8RA | | | | | | | | | | | | | | |
| 35292_at | D6S81E | Z37166 | 6 | Pass | 29.83 | 5.04 | 13 | Pass | TRUE | FALSE | FALSE | 9.38 | 3.18 | HLA-B associated | 6p21.3 | | | | | | | | | | | | | |
| | | | | | | | | | | | | | | transcript-1, D6S81E | | | | | | | | | | | | | | |
| 33438_at | WBP2 | AL049981 | 6 | Pass | 59 50 | 19.79 | 13 | Pass | TRUE | FALSE | FALSE | 18 77 | 3.17 | WW domain binding | 17q25 | | | | | | | | | | | | | |
| | | | | | | | | | | | | | | protein 2, WBP2 | | | | | | | | | | | | | | |
| 36372_at | HK3 | U51333 | 6 | Pass | 53 83 | 35.92 | 12 | Pass | TRUE | FALSE | FALSE | 17.00 | 3.17 | hexokinase 3 (white cell). 5q35 2 | | | | | | | | | | | | | | |
| | | | | | | | | | | | | | | HK3 | | | | | | | | | | | | | | |
| 39182_at EMP3 | U87947 | 6 | Pass | 155 83 | 50 34 | 13 | Pass | TRUE | FALSE | FALSE | 49.23 | 3.17 | epithelial membrane | 19q13 3 | | | | | | | | | | | | | | |
| | | | | | | | | | | | | | | protein 3, EMP3 | | | | | | | | | | | | | | |
| 36229_at | IL17R | U58917 | 4 | Pass | 12 25 | 8.10 | 8 | Pass | TRUE | FALSE | FALSE | 3.88 | 3.16 | interleukin 17 receptor, | 22q11.1 | | | | | | | | | | | | | |
| | | | | | | | | | | | | | | IL17R | | | | | | | | | | | | | | |
| 33371_s_at | RAB31 | U59877 | 6 | Pass | 34 17 | 14.36 | 13 | Pass | TRUE | FALSE | FALSE | 10 85 | 3.15 | RAB31, member RAS | 18p11 13 | | | | | | | | | | | | | |
| | | | | | | | | | | | | | | oncogene family, RAB31 | | | | | | | | | | | | | | |
| 40332 at | 7-60 | AF109134 | 6 | Pass | 35 33 | 18.54 | 9 | Pass | TRUE | FALSE | FALSE | 11 22 | 3.15 | 7-60 protein, 22098 | 20q13 3 | | | | | | | | | | | | | |
| 810_at | ARHGEF1 | U64105 | 6 | Pass | 27 83 | 5.49 | 13 | Pass | TRUE | FALSE | FALSE | 8 85 | 3.15 | Rho guanine nucleotide | 19q13.13 | | | | | | | | | | | | | |
| | | | | | | | | | | | | | | exchange factor (GEF) 1. | | | | | | | | | | | | | | |
| | | | | | | | | | | | | | | ARHGEF1 | | | | | | | | | | | | | | |
| 36960_at | EDR2 | U89278 | 6 | Pass | 13.17 | 7.83 | 13 | Pass | TRUE | FALSE | FALSE | 4.23 | 3.11 | early development | 1 | | | | | | | | | | | | | |
| | | | | | | | | | | | | | | regulator 2 (homolog of | | | | | | | | | | | | | | |
| | | | | | | | | | | | | | | polyhomeotic 2); EDR2 | | | | | | | | | | | | | | |
| 36785 | _at | HSPB1 | Z23090 | 6 | Pass | 22 67 | 9.07 | 13 | Pass | TRUE | FALSE | FALSE | 7 31 | 3.10 | heat shock 27kD protein 1, | 7q | | | | | | | | | | | | |
| | | | | | | | | | | | | | | HSPB1 | | | | | | | | | | | | | | |
| 39082 at | ANXA6 | Y00097 | 6 | Pass | 62.00 | 12.18 | 13 | Pass | TRUE | FALSE | FALSE | 20 15 | 3.08 | annexin A6, ANXA6 | 5q32-q34 | | | | | | | | | | | | | |
| 39400_at | KIAA1055 | AB028978 | 6 | Pass | 11 33 | 4 27 | 13 | Pass | TRUE | FALSE | FALSE | 3 69 | 3.07 | KIAA1055 protein, | 15 | | | | | | | | | | | | | |
| | | | | | | | | | | | | | | KIAA1055 | | | | | | | | | | | | | | |
| 41106_at | KCNN4 | AF022797 | 5 | Pass | 10 20 | 5 36 | 9 | Pass | TRUE | FALSE | FALSE | 3 33 | 3.06 | potassium | 19q13 2 | | | | | | | | | | | | | |
| | | | | | | | | | | | | | | intermediate/small | | | | | | | | | | | | | | |
| | | | | | | | | | | | | | | conductance calcium- | | | | | | | | | | | | | | |
| | | | | | | | | | | | | | | activated channel, | | | | | | | | | | | | | | |
| | | | | | | | | | | | | | | subfamily N, member 4, | | | | | | | | | | | | | | |
| | | | | | | | | | | | | | | KCNN4 | | | | | | | | | | | | | | |
| 39076_s_at | DRAP1 | A1991040 | 6 | Pass | 25 17 | 8 33 | 13 | Pass | TRUE | FALSE | FALSE | 8.23 | 3.06 | DR1-associated protein 1 | 11 | | | | | | | | | | | | | |
| | | | | | | | | | | | | | | (negative cofactor 2 | | | | | | | | | | | | | | |
| | | | | | | | | | | | | | | alpha), DRAP1 | | | | | | | | | | | | | | |
| 39112_at | USF2 | 6 | Pass | 15.00 | 4.43 | 13 | Pass | TRUE | FALSE | FALSE | 4 92 | 3.05 | upstream transcription | 19q13 | | | | | | | | | | | | | | |
| | | | | | | | | | | | | | | factor 2 c-los interacting, | | | | | | | | | | | | | | |
| | | | | | | | | | | | | | | USF2 | | | | | | | | | | | | | | |
| 38813 at | TSC2 | X75621 | 6 | Pass | 9.67 | 2 16 | 10 | Pass | TRUE | FALSE | FALSE | 3 20 | 3.02 | tubcrous sclerosis 2, TSC2 | 16p13 3 | | | | | | | | | | | | | |
| 34789_at | PI6 | S69272 | 6 | Pass | 23 83 | 10 76 | 13 | Pass | TRUE | FALSE | FALSE | 7.92 | 3.01 | protcase inhibitor 6 | 6p25 | | | | | | | | | | | | | |
| | | | | | | | | | | | | | | (placental thrombin | | | | | | | | | | | | | | |
| | | | | | | | | | | | | | | inhibitor), PI6 | | | | | | | | | | | | | | |
| 37145 at | GNLY | M85276 | 6 | Pass | 111 17 | 70.80 | 13 | Pass | TRUE | FALSE | FALSE | 37 00 | 3.00 | granulysum, GNLY | 2p12-q11 | | | | | | | | | | | | | |
| 34707_at | CHD3 | U91543 | 6 | Pass | 21.63 | 7 25 | 13 | Pass | TRUE | FALSE | FALSE | 7 31 | 2.99 | chromodomain hclease | 17p13 1 | | | | | | | | | | | | | |
| | | | | | | | | | | | | | | DNA binding protein 3, | | | | | | | | | | | | | | |
| | | | | | | | | | | | | | | CHD3 | | | | | | | | | | | | | | |
| 39069 at | CLCN7 | Z67743 | 6 | Pass | 29 83 | 6 77 | 11 | Pass | TRUE | FALSE | FALSE | 10.00 | 2.98 | chloride channel 7, | 16p13 | | | | | | | | | | | | | |
| 38063 at | UNK U00 | U00952 | 6 | Pass | 28 17 | 12 42 | 13 | Pass | TRUE | FALSE | FALSE | 9 46 | 2.98 | | | | | | | | | | | | | | | |
| 37281_at | KIAA0233 | D87071 | 6 | Pass | 36 33 | 11.18 | 13 | Pass | TRUE | FALSE | FALSE | 12.22 | 2.97 | KIAA0233 gene product, | 16 | | | | | | | | | | | | | |
| | | | | | | | | | | | | | | KIAA0233 | | | | | | | | | | | | | | |
| 38194_s_at | IGKV1D-8 | M63438 | 6 | Pass | 142 17 | 69.87 | 13 | Pass | TRUE | FALSE | FALSE | 47 92 | 2.97 | immunoglobulin kappa | 2p12 | | | | | | | | | | | | | |
| | | | | | | | | | | | | | | variable 1D-8, IGKV1D-8 | | | | | | | | | | | | | | |
| 37411_at | KIAA0050 | D30758 | 6 | Pass | 34 33 | 9 40 | 12 | Pass | TRUE | FALSE | FALSE | 11 58 | 2.96 | KIAA0050 gene product, | | | | | | | | | | | | | | |
| | | | | | | | | | | | | | | KIAA0050 | | | | | | | | | | | | | | |
| 36473_at | USP20 | AB023220 | 6 | Pass | 16 17 | 3.66 | 12 | Pass | TRUE | FALSE | FALSE | 5.50 | 2.94 | ubiqunitin specific protease | | | | | | | | | | | | | | |
| | | | | | | | | | | | | | | 20, USP20 | | | | | | | | | | | | | | |
| 36152_at | GDII | X79353 | 6 | Pass | 47 83 | 11.89 | 13 | Pass | TRUE | FALSE | FALSE | 16.31 | 2.93 | GDP dissociation inhibitor | Xq28 | | | | | | | | | | | | | |
| | | | | | | | | | | | | | | 1; GDII | | | | | | | | | | | | | | |
| 35530_f_at | IGL@ | X92997 | 6 | Pass | 30.17 | 22.56 | 11 | Pass | TRUE | FALSE | FALSE | 10 36 | 2.91 | immunoglobulin lambda | 22q11 1- | | | | | | | | | | | | | |
| | | | | | | | | | | | | | | locus, IGL@ | | | | | | | | | | | | | | |
| 40791_at | POLR2A | X63564 | 6 | Pass | 23 50 | 10 37 | 13 | Pass | TRUE | FALSE | FALSE | 8 08 | 2.91 | polymerase (RNA) II | 17p13 1 | | | | | | | | | | | | | |
| | | | | | | | | | | | | | | (DNA directed) | | | | | | | | | | | | | | |
| | | | | | | | | | | | | | | polypeptide A (220kD), | | | | | | | | | | | | | | |
| | | | | | | | | | | | | | | POLR2A | | | | | | | | | | | | | | |
| 41753 at | ACTN4 | U48734 | 6 | Pass | 30 83 | 12.92 | 13 | Pass | TRUE | FALSE | FALSE | 10 69 | 2.88 | actumin alpha 4, ACTN4 | 19q13 | | | | | | | | | | | | | |
| 33925_at | NRGN | X99076 | 6 | Pass | 220.17 | 53.81 | 13 | Pass | TRUE | FALSE | FALSE | 76 77 | 2.87 | neuroganin (protein | 11q24 | | | | | | | | | | | | | |
| | | | | | | | | | | | | | | kinase C substrate, RC3); | | | | | | | | | | | | | | |
| 39689_at | CST3 | AI362017 | 6 | Pass | 65.50 | 33 53 | 13 | Pass | TRUE | FALSE | FALSE | 22 85 | 2.87 | cystatin C (amyloid | 20p11.2 | | | | | | | | | | | | | |
| | | | | | | | | | | | | | | angiopathy and cerebral | | | | | | | | | | | | | | |
| | | | | | | | | | | | | | | hemorrhage), CST3 | | | | | | | | | | | | | | |
| 1107_s_at | ISG15 | M13755 | 6 | Pass | 39 67 | 28.62 | 13 | Pass | TRUE | FALSE | FALSE | 13 85 | 2.86 | interferon-stimulated | 1 | | | | | | | | | | | | | |
| | | | | | | | | | | | | | | protein, 15 kDa, ISG15 | | | | | | | | | | | | | | |
| 336_at | TBXA2R | D38081 | 5 | Pass | 11 80 | 2 59 | 7 | Pass | TRUE | FALSE | FALSE | 4 14 | 2.85 | thromboxane A2 receptor, | 19p13 3 | | | | | | | | | | | | | |
| | | | | | | | | | | | | | | TBXA2R | | | | | | | | | | | | | | |
| 31315 at | UNK D84 | D84143 | 5 | Pass | 22 00 | 16 09 | 8 | Pass | TRUE | FALSE | FALSE | 7 75 | 2.84 | | | | | | | | | | | | | | | |
| 319_g_at | HIFX | D64142 | 6 | Pass | 86.17 | 25 13 | 13 | Pass | TRUE | FALSE | FALSE | 30.46 2.83 | HI histone family, | | | | | | | | | | | | | | | |
| | | | | | | | | | | | | | | member X, HIFX | | | | | | | | | | | | | | |
| 36781_at | PI | X01683 | 6 | Pass | 101 67 | 57.50 | 13 | Pass | TRUE | FALSE | FALSE | 36.00 | 2.82 | protease inhibitor 1 (anti- | 14q32.1 | | | | | | | | | | | | | |
| | | | | | | | | | | | | | | elastase), alpha-1- | | | | | | | | | | | | | | |
| | | | | | | | | | | | | | | antitrypsin, PI | | | | | | | | | | | | | | |
| 41138_at | MIC2 | M16279 | 6 | Pass | 74.50 | 25.15 | 13 | Pass | TRUE | FALSE | FALSE | 26.38 | 2.82 | antigen identified by | Xp22.32, | | | | | | | | | | | | | |
| | | | | | | | | | | | | | | monoclonal antibodies | Yp11.3 | | | | | | | | | | | | | |
| | | | | | | | | | | | | | | 12E7, F21 and O13, MIC2 | | | | | | | | | | | | | | |
| 41198 at | GRN | AF055008 | 6 | Pass | 69.00 | 41 70 | 11 | Pass | TRUE | FALSE | FALSE | 24 45 | 2.82 | granulin, GRN | 17 | | | | | | | | | | | | | |
| 1294_at | UBE1L | L13852 | 6 | Pass | 22.83 | 3.92 | 10 | Pass | TRUE | FALSE | FALSE | 8 10 | 2.82 | ubiquitin-activating | 3p21 | | | | | | | | | | | | | |
| | | | | | | | | | | | | | | enzyme E1, like, UBE1L | | | | | | | | | | | | | | |
| 38894_g_at | NCF4 | AL008634 | 6 | Pass | 18 83 | 7 91 | 13 | Pass | TRUE | FALSE | FALSE | 6 69 | 2.81 | neutrophil cytosolic factor | 22q13 1 | | | | | | | | | | | | | |
| | | | | | | | | | | | | | | 4 (40kD), NCT4 | | | | | | | | | | | | | | |
| 40668 s at | CD6 | U34624 | 4 | Pass | 11.00 | 4 76 | 11 | Pass | TRUE | FALSE | FALSE | 3.91 | 2.81 | CD6 antigen, CD6 | 11q13 | | | | | | | | | | | | | |
| 38671_at | KIAA0620 | AB014520 | 6 | Pass | 11.00 | 10 24 | 11 | Pass | TRUE | FALSE | FALSE | 3.91 | 2.81 | KIAA0620 protein, | | | | | | | | | | | | | | |
| | | | | | | | | | | | | | | KIAA0620 | | | | | | | | | | | | | | |
| 38686_at | ATP6DV | X71490 | 6 | Pass | 31 17 | 16 81 | 11 | Pass | TRUE | FALSE | FALSE | 11 09 | 2.81 | Vacuolar protein-ATPasc, | | | | | | | | | | | | | | |
| | | | | | | | | | | | | | | subunit D, V-ATPasc, | | | | | | | | | | | | | | |
| | | | | | | | | | | | | | | subunit D, ATP6DV | | | | | | | | | | | | | | |
| 35132 at | MYO1E | X98411 | 6 | Pass | 94 33 | 40 45 | 13 | Pass | TRUE | FALSE | FALSE | 33 62 | 2.81 | myosin IE, MYO1E | | | | | | | | | | | | | | |
| 34405_at | USP5 | U47927 | 6 | Pass | 14 00 | 2 68 | 10 | Pass | TRUE | FALSE | FALSE | 5 00 | 2.80 | ubiquitin specific protease | 12p13 | | | | | | | | | | | | | |
| | | | | | | | | | | | | | | 5 (isopeptidase T), USP5 | | | | | | | | | | | | | | |
| 40667 at | CD6 | X60992 | 6 | Pass | 31 83 | 11 62 | 13 | Pass | TRUE | FALSE | FALSE | 11 38 | 2.80 | CD6 antigen, CD6 | 11q13 | | | | | | | | | | | | | |
| 3610_at | DD96 | U21049 | 6 | Pass | 21 67 | 9.00 | 12 | Pass | TRUE | FALSE | FALSE | 7 75 | 2.80 | epithelial protien up- | 1 | | | | | | | | | | | | | |
| | | | | | | | | | | | | | | regulated in carcinoma, | | | | | | | | | | | | | | |
| | | | | | | | | | | | | | | membrane associated | | | | | | | | | | | | | | |
| | | | | | | | | | | | | | | protein 17, DD96 | | | | | | | | | | | | | | |
| 39127_f_at | PPP2R4 | X73478 | 6 | Pass | 25 33 | 13 09 | 13 | Pass | TRUE | FALSE | FALSE | 9 08 | 2.79 | protein phosphatase 2A, | 9q34 | | | | | | | | | | | | | |
| | | | | | | | | | | | | | | regulatory subunit B′ (PR | | | | | | | | | | | | | | |
| | | | | | | | | | | | | | | 53), PPP2R4 | | | | | | | | | | | | | | |
| 36902_at | ARHG | X61587 | 6 | Pass | 42 67 | 17 57 | 13 | Pass | TRUE | FALSE | FALSE | 15 31 | 2.79 | ras homolog gene family, | 11p15 5- | | | | | | | | | | | | | |
| | | | | | | | | | | | | | | member G (rho G), ARHG | p15 4 | | | | | | | | | | | | | |
| 37387_r_at | KDELR1 | X55885 | 6 | Pass | 14 17 | 5 56 | 12 | Pass | TRUE | FALSE | FALSE | 5 08 | 2.79 | KDEL (Lys-Asp-Glu-Leu) | 19q13 3 | | | | | | | | | | | | | |
| | | | | | | | | | | | | | | endoplasmic retticulum | | | | | | | | | | | | | | |
| | | | | | | | | | | | | | | protein retention receptor | | | | | | | | | | | | | | |
| | | | | | | | | | | | | | | 1, KDELR1 | | | | | | | | | | | | | | |
| 33535_at | P2RX1 | U45448 | 6 | Pass | 9 17 | 4 17 | 10 | Pass | TRUE | FALSE | FALSE | 3 30 | 2.78 | purinergic receptor P2X, | 17p | | | | | | | | | | | | | |
| | | | | | | | | | | | | | | ligand-gated ion channel, | | | | | | | | | | | | | | |
| | | | | | | | | | | | | | | 1, P2RX1 | | | | | | | | | | | | | | |
| 36940_at | TIAF1 | D06970 | 6 | Pass | 5 83 | 1.83 | 10 | Pass | TRUE | FALSE | FALSE | 2 10 | 2.78 | TGFB1-induced anti- | 17 | | | | | | | | | | | | | |
| | | | | | | | | | | | | | | apoptotic factor 1; TIAF1 | | | | | | | | | | | | | | |
| 39770_at | KIAA0250 | D87437 | 6 | Pass | 8 83 | 3 97 | 11 | Pass | TRUE | FALSE | FALSE | 3 18 | 2.78 | KIAA0250 gene product, | | | | | | | | | | | | | | |
| | | | | | | | | | | | | | | KIAA0250 | | | | | | | | | | | | | | |
| 39119_s_at | NK4 | AA631972 | 6 | Pass | 79 33 | 22 00 | 13 | Pass | TRUE | FALSE | FALSE | 28.62 | 2.77 | natural killer cell transcript | 16p13 3 | | | | | | | | | | | | | |
| | | | | | | | | | | | | | | 4, NR4 | | | | | | | | | | | | | | |
| 41850_s_at | DIPA | U63825 | 6 | Pass | 12 33 | 5.39 | 11 | Pass | TRUE | FALSE | FALSE | 4 45 | 2.77 | hepatitis delta anyigen- | 11 | | | | | | | | | | | | | |
| | | | | | | | | | | | | | | interacting protein A: | | | | | | | | | | | | | | |
| 36843_at | SIPA1 | AB005666 | 6 | Pass | 12 83 | 5 67 | 11 | Pass | TRUE | FALSE | FALSE | 4 64 | 2.77 | signal-induced | 11q13.3 | | | | | | | | | | | | | |
| | | | | | | | | | | | | | | proliferation-associated | | | | | | | | | | | | | | |
| 38584_at | IFIT4 | AF026939 | 6 | Pass | 14 33 | 12 68 | 11 | Pass | TRUE | FALSE | FALSE | 5 18 | 2.77 | interferon-induced protein | 10q24 | | | | | | | | | | | | | |
| | | | | | | | | | | | | | | with tetratricopeptide | | | | | | | | | | | | | | |
| | | | | | | | | | | | | | | repeats 4, IFIT4 | | | | | | | | | | | | | | |
| 40955_at | UNK_U79 | U79287 | 5 | Pass | 7.60 | 2 70 | 8 | Pass | TRUE | FALSE | FALSE | 2 75 | 2.76 | prostate tumor over | | | | | | | | | | | | | | |
| | | | | | | | | | | | | | | expressed gene 1, PIOV1 | | | | | | | | | | | | | | |
| 35653_at | GPS2 | U28963 | 6 | Pass | 24.00 | 6 39 | 13 | Pass | TRUE | FALSE | FALSE | 8 69 | 2.76 | G protein pathway | | | | | | | | | | | | | | |
| | | | | | | | | | | | | | | suppressor 2, GFS2 | | | | | | | | | | | | | | |
| 181_g_at | UNK_S82 | S82470 | 6 | Pass | 26 17 | 15 45 | 10 | Pass | TRUE | FALSE | FALSE | 9 50 | 2.75 | BB1 = malignant cell | | | | | | | | | | | | | | |
| | | | | | | | | | | | | | | expression-enhanced | | | | | | | | | | | | | | |
| | | | | | | | | | | | | | | gene/tumor progression- | | | | | | | | | | | | | | |
| | | | | | | | | | | | | | | enhanced gene (human, | | | | | | | | | | | | | | |
| | | | | | | | | | | | | | | UM-UC-9 bladder | | | | | | | | | | | | | | |
| | | | | | | | | | | | | | | carcinoma cell line | | | | | | | | | | | | | | |
| 38445_at | ARHGEF1 | Y09160 | 6 | Pass | 24 33 | 6 77 | 13 | Pass | TRUE | FALSE | FALSE | 8.85 | 2.75 | Rho guanine nucleotide | 19q13 13 | | | | | | | | | | | | | |
| | | | | | | | | | | | | | | exchange factor (GEF) 1, | | | | | | | | | | | | | | |
| | | | | | | | | | | | | | | ARHGEF1 | | | | | | | | | | | | | | |
| 37992_s_at | ATP5D | AI436567 | 6 | Pass | 34.83 | 13 91 | 13 | Pass | TRUE | FALSE | FALSE | 12.69 | 2.74 | AIP synthase, H+ | | | | | | | | | | | | | | |
| | | | | | | | | | | | | | | transporting, | | | | | | | | | | | | | | |
| | | | | | | | | | | | | | | mitochondrial F1 complex, | | | | | | | | | | | | | | |
| | | | | | | | | | | | | | | delta subunit ATP5D | | | | | | | | | | | | | | |
| 36780_at | CLU | M25915 | 6 | Pass | 248.17 | 77.71 | 13 | Pass | TRUE | FALSE | FALSE | 90.46 | 2.74 | clusterin (complement | 8p21-p12 | | | | | | | | | | | | | |
| | | | | | | | | | | | | | | lysis inhibitor, SP-40, 40 | | | | | | | | | | | | | | |
| | | | | | | | | | | | | | | sulfated glycoprotein 2, | | | | | | | | | | | | | | |
| | | | | | | | | | | | | | | testosterone-repressed | | | | | | | | | | | | | | |
| | | | | | | | | | | | | | | prostate message 2. | | | | | | | | | | | | | | |
| 38798_s_at | G2AD | AI741833 | 6 | Pass | 16.67 | 3 27 | 13 | Pass | TRUE | FALSE | FALSE | 6 08 | 2.74 | adaptor-related protein | | | | | | | | | | | | | | |
| | | | | | | | | | | | | | | complex 1, gamma 2 | | | | | | | | | | | | | | |
| | | | | | | | | | | | | | | subunit, AP1G2 | | | | | | | | | | | | | | |
| 35773_t_at | NDUFB7 | 6 | Pass | 12.33 | 6 35 | 10 | Pass | TRUE | FALSE | FALSE | 4 50 | 2.74 | NADH dehydrogenase | | | | | | | | | | | | | | | |
| | | | | | | | | | | | | | | (ubiquinone) 1 beta | | | | | | | | | | | | | | |
| | | | | | | | | | | | | | | subcomplex, 7 (18kD, | | | | | | | | | | | | | | |
| | | | | | | | | | | | | | | B18), NDUFB7 | | | | | | | | | | | | | | |
| 36979_at | SLC2A3 | M20681 | 6 | Pass | 39.83 | 11 44 | 13 | Pass | TRUE | FALSE | FALSE | 14 54 | 2.74 | solute cancer family 2 | 12p13 3 | | | | | | | | | | | | | |
| | | | | | | | | | | | | | | (facilitated glucose | | | | | | | | | | | | | | |
| | | | | | | | | | | | | | | transporter), member 3, | | | | | | | | | | | | | | |
| | | | | | | | | | | | | | | SLC2A3 | | | | | | | | | | | | | | |
| 38517_at | ISGF3G | M87503 | 5 | Pass | 43.20 | 12 15 | 13 | Pass | TRUE | FALSE | FALSE | 15 77 | 2.74 | interferon-stimulated | 14q11 2 | | | | | | | | | | | | | |
| | | | | | | | | | | | | | | transcription factor 3, | | | | | | | | | | | | | | |
| | | | | | | | | | | | | | | gamma (48kD), ISGF3G | | | | | | | | | | | | | | |
| 41161_at | DAXX | AB015051 | 6 | Pass | 33 50 | 15.22 | 13 | Pass | TRUE | FALSE | FALSE | 12 23 | 2.74 | death-associated protein 6, | 6p21.3 | | | | | | | | | | | | | |
| | | | | | | | | | | | | | | DAXX | | | | | | | | | | | | | | |
| 37591_at | UCP2 | U94592 | 6 | Pass | 61.00 | 19.28 | 13 | Pass | TRUE | FALSE | FALSE | 22 31 | 2.73 | uncoupling protein 2 | 11q13 | | | | | | | | | | | | | |
| | | | | | | | | | | | | | | (mitochondrial, proton | | | | | | | | | | | | | | |
| | | | | | | | | | | | | | | carrier), UCP2 | | | | | | | | | | | | | | |
| 38391_at | CAPG | M94345 | 6 | Pass | 37 00 | 18.49 | 13 | Pass | TRUE | FALSE | FALSE | 13 54 | 2.73 | capping protein (actin | 3cen-q34 | | | | | | | | | | | | | |
| | | | | | | | | | | | | | | filament), gelsolin-like; | | | | | | | | | | | | | | |
| | | | | | | | | | | | | | | CAPG | | | | | | | | | | | | | | |
| 33855_at | GRB2 | M96995 | 6 | Pass | 16 17 | 6.97 | 13 | Pass | TRUE | FALSE | FALSE | 5.92 | 2.73 | growth factor receptor- | 17q24- | | | | | | | | | | | | | |
| | | | | | | | | | | | | | | bound protein 2, GRB2 | q25 | | | | | | | | | | | | | |
| 35626_at | SGSH | U30894 | 6 | Pass | 33 17 | 9.20 | 13 | Pass | TRUE | FALSE | FALSE | 12 15 | 2.73 | N-sulfoglucosamine | 17q25 3 | | | | | | | | | | | | | |
| | | | | | | | | | | | | | | sulfohydiolase | | | | | | | | | | | | | | |
| | | | | | | | | | | | | | | (sulfamidase), SGSH | | | | | | | | | | | | | | |
| 33916_at | I-1 | AB023192 | 6 | Pass | 23 00 | 9 14 | 9 | Pass | TRUE | FALSE | FALSE | 8 44 | 2.72 | imidazoline receptor | 3p21 1 | | | | | | | | | | | | | |
| | | | | | | | | | | | | | | candidate, I-1 | | | | | | | | | | | | | | |
| 37100_at | CI7ORF1B | AJ008112 | 6 | Pass | 22 17 | 6 55 | 13 | Pass | TRUE | FALSE | FALSE | 8 15 | 2.72 | chromosome 17 open | 17q21 | | | | | | | | | | | | | |
| | | | | | | | | | | | | | | reading france 1B, | | | | | | | | | | | | | | |
| | | | | | | | | | | | | | | CI7ORF1B | | | | | | | | | | | | | | |
| 36554_at | ASMTL | Y15521 | 6 | Pass | 14 67 | 7 84 | 7 | Pass | TRUE | FALSE | FALSE | 5 43 | 2.70 | acetylserotonim O- | Xp22 3, | | | | | | | | | | | | | |
| | | | | | | | | | | | | | | methyltransferase-like, | Yp11 3, | | | | | | | | | | | | | |
| | | | | | | | | | | | | | | ASMTL | | | | | | | | | | | | | | |
| 31870_at | CD37 | X14046 | 6 | Pass | 138 67 | 42 00 | 13 | Pass | TRUE | FALSE | FALSE | 51 38 | 2.70 | CD37 antigen, CD37 | 19p13- | | | | | | | | | | | | | |
| | | | | | | | | | | | | | | | q13 4 | | | | | | | | | | | | | |
| 41047 at | UNK AI88 | AI885170 | 6 | Pass | 36 50 | 8.19 | 13 | Pass | TRUE | FALSE | FALSE | 13 54 | 2.70 | | | | | | | | | | | | | | | |
| 36097_at | ETR101 | M62831 | 6 | Pass | 105.00 | 35 94 | 13 | Pass | TRUE | FALSE | FALSE | 39.00 | 2.69 | immediate early protein, | 19 | | | | | | | | | | | | | |
| | | | | | | | | | | | | | | ETR101 | | | | | | | | | | | | | | |
| 35813 at | TRN-SR | AA192359 | 6 | Pass | 15.17 | 8 38 | 11 | Pass | TRUE | FALSE | FALSE | 5.64 | 2.69 | transportin-SR, TRN-SR | 7 | | | | | | | | | | | | | |
| 41160_at | UNK_AC0 | AC005943 | 6 | Pass | 8.67 | 5.75 | 9 | Pass | TRUE | FALSE | FALSE | 3.22 | 2.69 | methyl-CpG binding | 19p13 3 | | | | | | | | | | | | | |
| | | | | | | | | | | | | | | domain protein 3; MBD3 | | | | | | | | | | | | | | |
| 38118_at | SHC1 | U73377 | 6 | Pass | 26.17 | 10.82 | 13 | Pass | TRUE | FALSE | FALSE | 9 77 | 2.68 | SHC (Src homology 2 | 1q21 | | | | | | | | | | | | | |
| | | | | | | | | | | | | | | domain-containing) | | | | | | | | | | | | | | |
| | | | | | | | | | | | | | | transforming protein 1, | | | | | | | | | | | | | | |
| | | | | | | | | | | | | | | SHC1 | | | | | | | | | | | | | | |
| 38997_at | SLC25A1 | X96924 | 6 | Pass | 10 17 | 8.04 | 10 | Pass | TRUE | FALSE | FALSE | 3 80 | 2.68 | solute carrier family 25 | 22q11 21 | | | | | | | | | | | | | |
| | | | | | | | | | | | | | | (mitochondrial carrier; | | | | | | | | | | | | | | |
| | | | | | | | | | | | | | | citrate transporter), | | | | | | | | | | | | | | |
| | | | | | | | | | | | | | | member 1; SLC25A1 | | | | | | | | | | | | | | |
| 371_at | IRF3 | Z56281 | 6 | Pass | 15.67 | 3.88 | 7 | Pass | TRUE | FALSE | FALSE | 5 86 | 2.67 | interferon regulatory factor | 19q13 3- | | | | | | | | | | | | | |
| | | | | | | | | | | | | | | 3, IRF3 | q13 4 | | | | | | | | | | | | | |
| 35823_at | PPIB | M63573 | 6 | Pass | 75 00 | 22.64 | 13 | Pass | TRUE | FALSE | FALSE | 2808 | 2.67 | peptidylprolyl isomertase B | 15q21- | | | | | | | | | | | | | |
| | | | | | | | | | | | | | | (cyclophilm B); PPIB | q22 | | | | | | | | | | | | | |
| 32761_at | KIAA0324 | AB002322 | 6 | Pass | 35 33 | 7.58 | 13 | Pass | TRUE | FALSE | FALSE | 13 23 | 2.67 | RNA binding protein; AT- | 10p13 3 | | | | | | | | | | | | | |
| | | | | | | | | | | | | | | rich element binding | | | | | | | | | | | | | | |
| | | | | | | | | | | | | | | factor, SRM300 | | | | | | | | | | | | | | |
| 33908_at | CAPN1 | X04366 | 6 | Pass | 56.50 | 21.92 | 13 | Pass | TRUE | FALSE | FALSE | 21.23 | 2.66 | calpain, large polypeptide | 11q13 | | | | | | | | | | | | | |
| | | | | | | | | | | | | | | L1: CAPN1 | | | | | | | | | | | | | | |
| 38597_f_at | SLCIIA1 | D50402 | 4 | Pass | 9 50 | 4 12 | 7 | Pass | TRUE | FALSE | FALSE | 3 57 | 2.66 | solute carrier family 11 | 2q35 | | | | | | | | | | | | | |
| | | | | | | | | | | | | | | (proton-coupled divalent | | | | | | | | | | | | | | |
| | | | | | | | | | | | | | | metal ion transporters), | | | | | | | | | | | | | | |
| | | | | | | | | | | | | | | member 1-SLCIIA1 | | | | | | | | | | | | | | |
| 36675 r at | PFN1 | J03191 | 6 | Pass | 174 17 | 57 46 | 13 | Pass | TRUE | FALSE | FALSE | 65 54 | 2.66 | profilm 1, PFN1 | 17p13 3 | | | | | | | | | | | | | |
| 32836_at | AGPAT1 | U56417 | 6 | Pass | 25 33 | 2 80 | 13 | Pass | TRUE | FALSE | FALSE | 9 54 | 2.66 | 1-acylglycerol-3- | 6p21.3 | | | | | | | | | | | | | |
| | | | | | | | | | | | | | | phosphate O- | | | | | | | | | | | | | | |
| | | | | | | | | | | | | | | acyltransferase 1 | | | | | | | | | | | | | | |
| | | | | | | | | | | | | | | (lysophosphatidic acid | | | | | | | | | | | | | | |
| | | | | | | | | | | | | | | acyltransferase, alpha); | | | | | | | | | | | | | | |
| 38417_at | AMPD2 | M91029 | 6 | Pass | 27 00 | 10 08 | 12 | Pass | TRUE | FALSE | FALSE | 10 17 | 2.66 | adenosine monophosphate | 1p13.3 | | | | | | | | | | | | | |
| | | | | | | | | | | | | | | deaminase 2 (isoform L), | | | | | | | | | | | | | | |
| | | | | | | | | | | | | | | AMPD2 | | | | | | | | | | | | | | |
| 39061_at | BST2 | D28137 | 6 | Pass | 59.33 | 33 66 | 13 | Pass | TRUE | FALSE | FALSE | 22 38 | 2.65 | bone marrow stromal cell | 19p13 2 | | | | | | | | | | | | | |
| | | | | | | | | | | | | | | antigen 2; BST2 | | | | | | | | | | | | | | |
| 38442_at | MFAP2 | U19718 | 4 | Pass | 16 25 | 1.71 | 7 | Pass | TRUE | FALSE | FALSE | 6 14 | 2.65 | microfibrillar-associated | 1p36 1- | | | | | | | | | | | | | |
| | | | | | | | | | | | | | | protein 2; MFAP2 | | | | | | | | | | | | | | |
| 35629 at | UNK AL0 | AL022238 | 6 | Pass | 23 00 | 12 21 | 11 | Pass | TRUE | FALSE | FALSE | 8 73 | 2.64 | | | | | | | | | | | | | | | |
| 36766_at | RNASE2 | X55988 | 6 | Pass | 37.50 | 29 43 | 13 | Pass | TRUE | FALSE | FALSE | 14 23 | 2.64 | ribonuclease, RNase A | 14q24- | | | | | | | | | | | | | |
| | | | | | | | | | | | | | | family, 2 (liver, eosinophil- | q31 | | | | | | | | | | | | | |
| | | | | | | | | | | | | | | derived neuotoxin), | | | | | | | | | | | | | | |
| | | | | | | | | | | | | | | RNASE2 | | | | | | | | | | | | | | |
| 34670_at | MAN2B1 | U60899 | 6 | Pass | 31.00 | 10 68 | 13 | Pass | TRUE | FALSE | FALSE | 11 77 | 2.63 | mannosidase, alpha, class | 19cen- | | | | | | | | | | | | | |
| | | | | | | | | | | | | | | 2B, member 1; MAN2B1 | q13 1 | | | | | | | | | | | | | |
| 41164_at | IGHM | X67301 | 6 | Pass | 134 17 | 51.81 | 13 | Pass | TRUE | FALSE | FALSE | 51 00 | 2.63 | immunoglobulin heavy | 14q32 33 | | | | | | | | | | | | | |
| | | | | | | | | | | | | | | constant mu, IGGM | | | | | | | | | | | | | | |
| 1693_s_at | TMP1 | D11139 | 6 | Pass | 51.50 | 23 10 | 13 | Pass | TRUE | FALSE | FALSE | 19 62 | 2.63 | tissue inhibitor of | Xp11 3- | | | | | | | | | | | | | |
| | | | | | | | | | | | | | | metalloprotenase 1 | p11 23 | | | | | | | | | | | | | |
| | | | | | | | | | | | | | | (erythroid potentiating | | | | | | | | | | | | | | |
| | | | | | | | | | | | | | | activity, collagenase | | | | | | | | | | | | | | |
| | | | | | | | | | | | | | | inhibitor); TIMP1 | | | | | | | | | | | | | | |
| 36661 s at | CD14 | X06882 | 6 | Pass | 156 17 | 111 56 | 13 | Pass | TRUE | FALSE | FALSE | 59 54 | 2.62 | CD14 antigen, CD14 | 5q31 t | | | | | | | | | | | | | |
| 37386_t_at | KDELR1 | X55885 | 6 | Pass | 38.50 | 24.19 | 13 | Pass | TRUE | FALSE | FALSE | 14 69 | 2.62 | KDEL (Lys-Asp-Glu-Leu) | 19q13 3 | | | | | | | | | | | | | |
| | | | | | | | | | | | | | | endoplasmic reticulum | | | | | | | | | | | | | | |
| | | | | | | | | | | | | | | protein retention receptor | | | | | | | | | | | | | | |
| | | | | | | | | | | | | | | 1, KDELR1 | | | | | | | | | | | | | | |
| 39358_at | NCOR2 | U37146 | 6 | Pass | 22.17 | 10.13 | 13 | Pass | TRUE | FALSE | FALSE | 8 46 | 2.62 | nuclear receptor co- | 12q24 | | | | | | | | | | | | | |
| | | | | | | | | | | | | | | repressor 2, NCOR2 | | | | | | | | | | | | | | |
| 31431_at | FCGRT | U12255 | 6 | Pass | 32 83 | 18.49 | 13 | Pass | TRUE | FALSE | FALSE | 12 54 | 2.62 | Fe ligament of IgG, | 19q13 3 | | | | | | | | | | | | | |
| | | | | | | | | | | | | | | receptor, transposter, | | | | | | | | | | | | | | |
| | | | | | | | | | | | | | | alpha, FCGRT | | | | | | | | | | | | | | |
| 40725_at | GOSR1 | AF047438 | 6 | Pass | 9 67 | 4 27 | 13 | Pass | TRUE | FALSE | FALSE | 3 69 | 2.62 | golgi SNAP receptor | 17q11 | | | | | | | | | | | | | |
| | | | | | | | | | | | | | | complex member 1. | | | | | | | | | | | | | | |
| 1754_at | DAXX | AF006041 | 6 | Pass | 14 83 | 4 92 | 12 | Pass | TRUE | FALSE | FALSE | 5 67 | 2.62 | death-associated protein 6, | 6p21.3 | | | | | | | | | | | | | |
| | | | | | | | | | | | | | | DAXX | | | | | | | | | | | | | | |
| 37179_at | NFE2 | S77763 | 6 | Pass | 15 50 | 8 02 | 13 | Pass | TRUE | FALSE | FALSE | 5 92 | 2.62 | nuclear factor (erythroid- | 12q13 | | | | | | | | | | | | | |
| | | | | | | | | | | | | | | derived 2), 45kD, NFE2 | | | | | | | | | | | | | | |
| 1067_at | FLT3LG | U03858 | 6 | Pass | 8 50 | 1 87 | 12 | Pass | TRUE | FALSE | FALSE | 3 25 | 2.62 | fms-related tyrosine kinase | 19q13 3 | | | | | | | | | | | | | |
| | | | | | | | | | | | | | | 3 ligand, FLT3LG | | | | | | | | | | | | | | |
| 38547_at | ITGAL | Y00796 | 6 | Pass | 29 33 | 7 63 | 13 | Pass | TRUE | FALSE | FALSE | 11 23 | 2.61 | integrin, alpha L (antigen | 16p11 2 | | | | | | | | | | | | | |
| | | | | | | | | | | | | | | CDI1A (p180), | | | | | | | | | | | | | | |
| | | | | | | | | | | | | | | lymphocyte function- | | | | | | | | | | | | | | |
| | | | | | | | | | | | | | | associated antigen 1; alpha | | | | | | | | | | | | | | |
| | | | | | | | | | | | | | | polypeptide), ITGAL | | | | | | | | | | | | | | |
| 38730_at | KIAA0864 | AB020671 | 6 | Pass | 22 50 | 3 94 | 13 | Pass | TRUE | FALSE | FALSE | 8 62 | 2.61 | KIAA0864 protein, | | | | | | | | | | | | | | |
| | | | | | | | | | | | | | | KIAA0864 | | | | | | | | | | | | | | |
| 33841_at | EIF5 | R48209 | 6 | Pass | 10.83 | 3 87 | 12 | Pass | TRUE | FALSE | FALSE | 4 17 | 2.60 | kinesin-like 5 (mitotic | | | | | | | | | | | | | | |
| | | | | | | | | | | | | | | kinesin-like protein 1): | | | | | | | | | | | | | | |
| | | | | | | | | | | | | | | KNSL5 | | | | | | | | | | | | | | |
| 503_at | POLR2L | U37690 | 5 | Pass | 41.60 | 10.53 | 13 | Pass | TRUE | FALSE | FALSE | 16.00 | 2.60 | polymerase (RNA) II | 11p15 | | | | | | | | | | | | | |
| | | | | | | | | | | | | | | (DNA directed) | | | | | | | | | | | | | | |
| | | | | | | | | | | | | | | polypeptide L (7 6kD)- | | | | | | | | | | | | | | |
| | | | | | | | | | | | | | | POLR2L | | | | | | | | | | | | | | |
| 39050_at | PABPN1 | AF026029 | 6 | Pass | 24 00 | 7.40 | 13 | Pass | TRUE | FALSE | FALSE | 9.23 | 2.60 | poly(A)-binding protein, | 14q11 2- | | | | | | | | | | | | | |
| | | | | | | | | | | | | | | nuclear 1, PABPN1 | | | | | | | | | | | | | | |
| 947_at | MCM7 | D55716 | 6 | Pass | 11 17 | 4.22 | 10 | Pass | TRUE | FALSE | FALSE | 4.30 | 2.60 | mumchromosome | 7q21 3- | | | | | | | | | | | | | |
| | | | | | | | | | | | | | | maintenance deficient (S | q22 1 | | | | | | | | | | | | | |
| | | | | | | | | | | | | | | ceravisae) 7, MCM7 | | | | | | | | | | | | | | |
| 33836 at | NPIP | AC002045 | 6 | Pass | 12 33 | 2.25 | 8 | Pass | TRUE | FALSE | FALSE | 4 75 | 2.60 | nuclear pore complex | 16p13- | | | | | | | | | | | | | |
| | | | | | | | | | | | | | | interacting protein, NPIP | p11 | | | | | | | | | | | | | |
| 39910_at | UNK_AA6 | AA63800 | 6 | Pass | 10 17 | 2.64 | 13 | Pass | TRUE | FALSE | FALSE | 3 92 | 2.59 | hypothetical protein, | | | | | | | | | | | | | | |
| | | | | | | | | | | | | | | LOC51257 | | | | | | | | | | | | | | |
| 32091_at | UNK_AB0 | AB007915 | 6 | Pass | 11 00 | 6.72 | 12 | Pass | TRUE | FALSE | FALSE | 4 25 | 2.59 | KIAA0446 gene product, | 1 | | | | | | | | | | | | | |
| | | | | | | | | | | | | | | KIAA0446 | | | | | | | | | | | | | | |
| 1131_at | MAP2K2 | L11285 | 6 | Pass | 24 83 | 5.19 | 13 | Pass | TRUE | FALSE | FALSE | 9 62 | 2.58 | mitogen-activated protein | 7q32 | | | | | | | | | | | | | |
| | | | | | | | | | | | | | | kinase kinsae 2, MAP2K2 | | | | | | | | | | | | | | |
| 40448_at | ZFP36 | M92843 | 6 | Pass | 43 83 | 24 09 | 13 | Pass | TRUE | FALSE | FALSE | 17.00 | 2.58 | zinc finger protein: 19q13 1 | | | | | | | | | | | | | | |
| | | | | | | | | | | | | | | homologous to Zfp-36 m | | | | | | | | | | | | | | |
| | | | | | | | | | | | | | | mouse, ZFP36 | | | | | | | | | | | | | | |
| 39280_at | TNRC5 | U80744 | 6 | Pass | 10 50 | 5.32 | 11 | Pass | TRUE | FALSE | FALSE | 4.09 | 2.57 | tinucleotide repeat | 1 | | | | | | | | | | | | | |
| | | | | | | | | | | | | | | containing 5-TNRC5 | | | | | | | | | | | | | | |
| 35674_at | PD12 | AB023211 | 4 | Pass | 10 25 | 6.13 | 7 | Pass | TRUE | FALSE | FALSE | 4 00 | 2.56 | peptidyl arginine | | | | | | | | | | | | | | |
| | | | | | | | | | | | | | | denunnase, type 11, PD12 | | | | | | | | | | | | | | |
| 198_g_at | NME3 | U29656 | 6 | Pass | 22 83 | 6.65 | 13 | Pass | TRUE | FALSE | FALSE | 8.92 | 2.56 | non-metastatic cells 3, | 16q13 | | | | | | | | | | | | | |
| | | | | | | | | | | | | | | protein expressed in. | | | | | | | | | | | | | | |
| | | | | | | | | | | | | | | NME3 | | | | | | | | | | | | | | |
| 31812_at | GMPR | M24470 | 6 | Pass | 19 17 | 7.63 | 12 | Pass | TRUE | FALSE | FALSE | 7.50 | 2.56 | guanosine monophosphate | 6p23 | | | | | | | | | | | | | |
| | | | | | | | | | | | | | | reductase, GMPR | | | | | | | | | | | | | | |
| 40296 at | UNK AL0 | AL023653 | 6 | Pass | 29 83 | 11 79 | 13 | Pass | TRUE | FALSE | FALSE | 11 69 | 2.55 | | | | | | | | | | | | | | | |
| 37285_at | ALAS2 | X60364 | 6 | Pass | 84 33 | 43.66 | 13 | Pass | TRUE | FALSE | FALSE | 33 08 | 2.55 | aminolevulinate, delta-, | Xp11 21 | | | | | | | | | | | | | |
| | | | | | | | | | | | | | | synthase 2 | | | | | | | | | | | | | | |
| | | | | | | | | | | | | | | (stderoblastic/hypochromic | | | | | | | | | | | | | | |
| | | | | | | | | | | | | | | amemia), ALAS2 | | | | | | | | | | | | | | |
| 36984_at | ATP6C | M62762 | 6 | Pass | 60 17 | 24 26 | 13 | Pass | TRUE | FALSE | FALSE | 23 62 | 2.55 | ATPase. H+ transporting, | 16p13 3 | | | | | | | | | | | | | |
| | | | | | | | | | | | | | | lysosomal (vacuolar | | | | | | | | | | | | | | |
| | | | | | | | | | | | | | | proton pump) 16kD, | | | | | | | | | | | | | | |
| 34532 at | UNK AF0 | AF035318 | 6 | Pass | 16 67 | 6 74 | 11 | Pass | TRUE | FALSE | FALSE | 6 55 | 2.55 | | | | | | | | | | | | | | | |
| 33251_at | KIAA0779 | AB018322 | 6 | Pass | 6 50 | 1.76 | 9 | Pass | TRUE | FALSE | FALSE | 2 56 | 2.54 | KIAA0779 protein, | | | | | | | | | | | | | | |
| | | | | | | | | | | | | | | KIAA0779 | | | | | | | | | | | | | | |
| 33860_at | KIAA0462 | AB007931 | 6 | Pass | 21 67 | 6 28 | 13 | Pass | TRUE | FALSE | FALSE | 8 54 | 2.54 | KIAA0462 protein, | 1 | | | | | | | | | | | | | |
| | | | | | | | | | | | | | | KIAA0462 | | | | | | | | | | | | | | |
| 30437_at | UNK_Z783 | Z78324 | 6 | Pass | 18 33 | 7.20 | 13 | Pass | TRUE | FALSE | FALSE | 7 23 | 2.54 | SH3 domain-containing | | | | | | | | | | | | | | |
| | | | | | | | | | | | | | | protein 6511, LOC51165 | | | | | | | | | | | | | | |
| 38601_at | RDA32 | AF061836 | 6 | Pass | 26 50 | 6 77 | 13 | Pass | TRUE | FALSE | FALSE | 10.46 | 2.53 | Ras association | 3p21 3 | | | | | | | | | | | | | |
| | | | | | | | | | | | | | | (RalGDS/AF-6) domain | | | | | | | | | | | | | | |
| | | | | | | | | | | | | | | family 1, RASSF1 | | | | | | | | | | | | | | |
| 32773 at | HLA-DQA | AA868382 | 6 | Pass | 57 83 | 23.45 | 13 | Pass | TRUE | FALSE | FALSE | 22.92 | 2.52 | major histocompatibility | 6p21.3 | | | | | | | | | | | | | |
| | | | | | | | | | | | | | | complex, class II, DQ | | | | | | | | | | | | | | |
| | | | | | | | | | | | | | | alpha 1, HLA-DQA1 | | | | | | | | | | | | | | |
| 33398_at | KIAA8670 | AB014570 | 6 | Pass | 29 67 | 13 52 | 9 | Pass | TRUE | FALSE | FALSE | 11.78 | 2.52 | KIAA0670 protein/actinus, | 14 | | | | | | | | | | | | | |
| | | | | | | | | | | | | | | KIAA0670 | | | | | | | | | | | | | | |
| 31622_f_at | MTIF | M10943 | 5 | Pass | 29.00 | 15 70 | 11 | Pass | TRUE | FALSE | FALSE | 11.55 | 2.51 | metallothionein 1F | 16q13 | | | | | | | | | | | | | |
| | | | | | | | | | | | | | | (functiotal), MTIF | | | | | | | | | | | | | | |
| 36678 at | TAGLN2 | D21261 | 6 | Pass | 175.00 | 42 08 | 13 | Pass | TRUE | FALSE | FALSE | 69 69 | 2.51 | transgelin 2: TAGLN2 | 1q21-q25 | | | | | | | | | | | | | |
| 33659_at | CFL1 | X95404 | 6 | Pass | 207.83 | 56 94 | 13 | Pass | TRUE | FALSE | FALSE | 82.02 | 2.51 | coflin 1 (non-muscle), | 11q13 | | | | | | | | | | | | | |
| | | | | | | | | | | | | | | CFL1 | | | | | | | | | | | | | | |
| 497_at | CLN3 | U32680 | 6 | Pass | 18.00 | 5.88 | 11 | Pass | TRUE | FALSE | FALSE | 7.18 | 2.51 | ceroid-lipofuscinosis, | 16p12 1 | | | | | | | | | | | | | |
| | | | | | | | | | | | | | | neuronal 3. juvenile | | | | | | | | | | | | | | |
| | | | | | | | | | | | | | | (Batten, Spielmeyer-Vogt | | | | | | | | | | | | | | |
| | | | | | | | | | | | | | | disease), CLN3 | | | | | | | | | | | | | | |
| 39704_s_at | HMGIY | L17131 | 5 | Pass | 20.40 | 4.77 | 7 | Pass | TRUE | FALSE | FALSE | 8.14 | 2.51 | high-nobility group | 6p21 | | | | | | | | | | | | | |
| | | | | | | | | | | | | | | (nonhistone chromosomal) | | | | | | | | | | | | | | |
| | | | | | | | | | | | | | | protein isoform I and Y, | | | | | | | | | | | | | | |
| | | | | | | | | | | | | | | HMGIY | | | | | | | | | | | | | | |
| 31432_g_at | FCGRT | U12255 | 6 | Pass | 57.33 | 28 74 | 12 | Pass | TRUE | FALSE | FALSE | 22 92 | 2.50 | Fe fragment of IgG, | 19q13.3 | | | | | | | | | | | | | |
| | | | | | | | | | | | | | | receptor, transporter, | | | | | | | | | | | | | | |
| | | | | | | | | | | | | | | alpha FC GRT | | | | | | | | | | | | | | |
| 31935_s_at | UNK_U75 | U75968 | 5 | Pass | 17 20 | 5 07 | 8 | Pass | TRUE | FALSE | FALSE | 6.88 | 2.50 | DEAD/H (Asp-Glu-Ala- | 12p11 | | | | | | | | | | | | | |
| | | | | | | | | | | | | | | Asp/His) box polypeptide | | | | | | | | | | | | | | |
| | | | | | | | | | | | | | | 11 (S. cerevisae CIIL1- | | | | | | | | | | | | | | |
| | | | | | | | | | | | | | | like helicase), DDX11 | | | | | | | | | | | | | | |
| 33965_at | HSPA6 | X51757 | 6 | Pass | 14.17 | 8 91 | Pass | TRUE | FALSE | FALSE | 5 67 | 2.50 | heat shock 70kD protein 6 | 1cen-qter | | | | | | | | | | | | | | |
| | | | | | | | | | | | | | | (HSP70B′), HSPA6 | | | | | | | | | | | | | | |
| 40867_at | PPP2RIA | J02902 | 5 | Pass | 30 40 | 5 94 | 12 | Pass | TRUE | FALSE | FALSE | 12 17 | 2.50 | protein phosphatase 2 | | | | | | | | | | | | | | |
| | | | | | | | | | | | | | | (formerly 2A), regulatory | | | | | | | | | | | | | | |
| | | | | | | | | | | | | | | subunit A (PR 65), alpha | | | | | | | | | | | | | | |
| | | | | | | | | | | | | | | isoform, PPP2RIA | | | | | | | | | | | | | | |
| 32824_at | CLN2 | AF039704 | 5 | Pass | 37 83 | 17 23 | 13 | Pass | TRUE | FALSE | FALSE | 15 15 | 2.50 | ceroid-lipofusinosis, | 11p15 | | | | | | | | | | | | | |
| | | | | | | | | | | | | | | nucuronal 2, late infantile | | | | | | | | | | | | | | |
| | | | | | | | | | | | | | | (Jansky-Brelschowsky | | | | | | | | | | | | | | |
| | | | | | | | | | | | | | | disease), CLN2 | | | | | | | | | | | | | | |
| 40098_at | EHD1 | AF001434 | 6 | Pass | 18 50 | 3 21 | 12 | Pass | TRUE | FALSE | FALSE | 7.42 | 2.49 | EII domain containing 1, | 11q13 | | | | | | | | | | | | | |
| | | | | | | | | | | | | | | EHD1 | | | | | | | | | | | | | | |
| 879_at | MX2 | M30818 | 6 | Pass | 16 50 | 9 14 | 13 | Pass | TRUE | FALSE | FALSE | 6.62 | 2.49 | myxovirus (influenza) | 21q22 3 | | | | | | | | | | | | | |
| | | | | | | | | | | | | | | resistance 2, homolog of | | | | | | | | | | | | | | |
| | | | | | | | | | | | | | | murine MX2 | | | | | | | | | | | | | | |
| 1790_s_at | CDK10 | X78342 | 6 | Pass | 18 50 | 6 16 | 7 | Pass | TRUE | FALSE | FALSE | 7 43 | 2.49 | cyclin-dependent kinase | 16q24 | | | | | | | | | | | | | |
| | | | | | | | | | | | | | | (CDC2-like) 10, CDK10 | | | | | | | | | | | | | | |
| 411_t_at | IFITM2 | X57351 | 6 | Pass | 51 67 | 27 91 | 13 | Pass | TRUE | FALSE | FALSE | 20 77 | 2.49 | interferon induced | | | | | | | | | | | | | | |
| | | | | | | | | | | | | | | transmembrane and protein 2 | | | | | | | | | | | | | | |
| | | | | | | | | | | | | | | 1-8D), IFITM2 | | | | | | | | | | | | | | |
| 915_at | IFIT1 | M24594 | 4 | Pass | 9 25 | 3 30 | 11 | Pass | TRUE | FALSE | FALSE | 3 73 | 2.48 | interferon-induced protein | 10q25- | | | | | | | | | | | | | |
| | | | | | | | | | | | | | | 56, IFIT1 | q26 | | | | | | | | | | | | | |
| 38361_g_at | RASGRP2 | A1688812 | 6 | Pass | 14 50 | 0 84 | 13 | Pass | TRUE | FALSE | FALSE | 5 85 | 2.48 | RAS guanyl releasing | 11q13 | | | | | | | | | | | | | |
| | | | | | | | | | | | | | | protein 2 (calcium and | | | | | | | | | | | | | | |
| | | | | | | | | | | | | | | DAG-regulated), | | | | | | | | | | | | | | |
| | | | | | | | | | | | | | | RASGRP2 | | | | | | | | | | | | | | |
| 38631_at | TNFAIP2 | M92357 | 6 | Pass | 43.83 | 20 08 | 13 | Pass | TRUE | FALSE | FALSE | 17 69 | 2.48 | tumor necrosis factor, | 14q32 | | | | | | | | | | | | | |
| | | | | | | | | | | | | | | alpha-induced protein 2, | | | | | | | | | | | | | | |
| | | | | | | | | | | | | | | TNFAIP2 | | | | | | | | | | | | | | |
| 33207_at | PRKR1 | AI095508 | 6 | Pass | 9 17 | 4 54 | 10 | Pass | TRUE | FALSE | FALSE | 3 70 | 2.48 | protein-kinase, interferon- | 13q32 | | | | | | | | | | | | | |
| | | | | | | | | | | | | | | inducible double stranded | | | | | | | | | | | | | | |
| | | | | | | | | | | | | | | RNA dependent inhibitor; | | | | | | | | | | | | | | |
| | | | | | | | | | | | | | | PRKR1 | | | | | | | | | | | | | | |
| 31673_s_at | CMAR | X65784 | 6 | Pass | 20 00 | 5 93 | 13 | Pass | TRUE | FALSE | FALSE | 8 08 | 2.48 | cell matrix adhesion | 16q16q24 | | | | | | | | | | | | | |
| | | | | | | | | | | | | | | regulator, spastic | 3 | | | | | | | | | | | | | |
| | | | | | | | | | | | | | | paraplegia 7, paraplegin | | | | | | | | | | | | | | |
| | | | | | | | | | | | | | | (pure and complicated | | | | | | | | | | | | | | |
| | | | | | | | | | | | | | | autosomal recessive). | | | | | | | | | | | | | | |
| 49099_at | LFP40 | AB014551 | 6 | Pass | 20 00 | 10.20 | 13 | Pass | TRUE | FALSE | FALSE | 8 08 | 2.48 | rho/iac guanine nucleotide | 1 | | | | | | | | | | | | | |
| | | | | | | | | | | | | | | exchange factar (GEF) 2, | | | | | | | | | | | | | | |
| | | | | | | | | | | | | | | ARHGEF2 | | | | | | | | | | | | | | |
| 34206_at | KIAA0782 | AB018325 | 6 | Pass | 20.17 8.66 | 13 | Pass | TRUE | FALSE | FALSE | 8 15 | 2.47 | KIAA0782 protein, | | | | | | | | | | | | | | | |
| | | | | | | | | | | | | | | KIAA06782 | | | | | | | | | | | | | | |
| 329_s_at | NUMA1 | Z11584 | 6 | Pass | 18 83 | 5 27 | 13 | Pass | TRUE | FALSE | FALSE | 7 62 | 2.47 | nuclear miototic apparatus | 11q13 | | | | | | | | | | | | | |
| | | | | | | | | | | | | | | protein 1 NUMA1 | | | | | | | | | | | | | | |
| 36447_at | FCN1 | S80990 | 6 | Pass | 246.17 | 113.27 | 13 | Pass | TRUE | FALSE | FALSE | 99 69 | 2.47 | ficolin | 9q34 | | | | | | | | | | | | | |
| | | | | | | | | | | | | | | (Collagen/fibrinogen | | | | | | | | | | | | | | |
| | | | | | | | | | | | | | | domain-containing) 1, | | | | | | | | | | | | | | |
| 41222_at | STAT6 | AF067575 | 6 | Pass | 55.83 | 30.42 | 13 | Pass | TRUE | FALSE | FALSE | 22.62 | 2.47 | signal transducer and | 12q13 | | | | | | | | | | | | | |
| | | | | | | | | | | | | | | activator of transcription 6, | | | | | | | | | | | | | | |
| | | | | | | | | | | | | | | interleukin-4 induced, | | | | | | | | | | | | | | |
| | | | | | | | | | | | | | | STAT6 | | | | | | | | | | | | | | |
| 35094_f_at | LILRA3 | AF025527 | 6 | Pass | 26.00 | 15.35 | 12 | Pass | TRUE | FALSE | FALSE | 10.58 | 2.46 | leukocyte immunoglobulin | 19q13.4 | | | | | | | | | | | | | |
| | | | | | | | | | | | | | | like receptor, subfamily A | | | | | | | | | | | | | | |
| | | | | | | | | | | | | | | (without IM domain), | | | | | | | | | | | | | | |
| | | | | | | | | | | | | | | member 3, LILRA3 | | | | | | | | | | | | | | |
| 33453_at | ATP6S1 | A1400326 | 6 | Pass | 51.00 | 12.95 | 13 | Pass | TRUE | FALSE | FALSE | 20 77 | 2.46 | ATPase, H+ transporting | Xq28 | | | | | | | | | | | | | |
| | | | | | | | | | | | | | | lysosomal (vacuolar | | | | | | | | | | | | | | |
| | | | | | | | | | | | | | | proton pump), subunit 1, | | | | | | | | | | | | | | |
| 41409_at | ICB-1 | AF044896 | 6 | Pass | 44 67 | 23 90 | 13 | Pass | TRUE | FALSE | FALSE | 18 23 | 2.45 | basement membrane- | | | | | | | | | | | | | | |
| | | | | | | | | | | | | | | induced gene, ICB-1 | | | | | | | | | | | | | | |
| 33887_at | HGS | D84064 | 6 | Pass | 16 83 | 3 66 | 8 | Pass | TRUE | FALSE | FALSE | 6 88 | 2.45 | human growth factor- | 17q25 | | | | | | | | | | | | | |
| | | | | | | | | | | | | | | regulated tyrosine kinase | | | | | | | | | | | | | | |
| | | | | | | | | | | | | | | substrate HGS | | | | | | | | | | | | | | |
| 36936_at | TSTA3 | U58766 | 6 | Pass | 10.17 | 1.60 | 13 | Pass | TRUE | FALSE | FALSE | 4 15 | 2.45 | tissue specific | 8q24 3 | | | | | | | | | | | | | |
| | | | | | | | | | | | | | | transplantation antigen | | | | | | | | | | | | | | |
| | | | | | | | | | | | | | | P35B, TSTA3 | | | | | | | | | | | | | | |
| 117_at | HSPA6 | X51757 | 5 | Pass | 10.40 | 4 22 | 8 | Pass | TRUE | FALSE | FALSE | 4 25 | 2.45 | heat shock 70kD protein 6 | 1cen-qter | | | | | | | | | | | | | |
| | | | | | | | | | | | | | | (HSP70B′), HSPA6 | | | | | | | | | | | | | | |
| 32211_at | PSMD13 | AB009398 | 6 | Pass | 10.83 | 7 28 | 7 | Pass | TRUE | FALSE | FALSE | 4.43 | 2.45 | protosome (prosome, | 11p15 5 | | | | | | | | | | | | | |
| | | | | | | | | | | | | | | macropain) 26S subunit, | | | | | | | | | | | | | | |
| | | | | | | | | | | | | | | non-ATPase, 13, PSMD13 | | | | | | | | | | | | | | |
| 41337 at | AES | AF072902 | 6 | Pass | 49.67 | 16 39 | 13 | Pass | TRUE | FALSE | FALSE | 20 31 | 2.45 | amino-terminal enhancer | 19q13 3 | | | | | | | | | | | | | |
| | | | | | | | | | | | | | | of split, AES | | | | | | | | | | | | | | |
| 33230_at | NMP200 | AJ131186 | 6 | Pass | 14.67 | 4.03 | 12 | Pass | TRUE | FALSE | FALSE | 6.00 | 2.44 | nuclear matrix protein | 11q12 2 | | | | | | | | | | | | | |
| | | | | | | | | | | | | | | NMP200 related to | | | | | | | | | | | | | | |
| | | | | | | | | | | | | | | spleing factor PRP19, | | | | | | | | | | | | | | |
| 39062_at | PPGB | AL008726 | 6 | Pass | 53 17 | 15.46 | 13 | Pass | TRUE | FALSE | FALSE | 21 77 | 2.44 | protective protein for beta- | 20q13 1 | | | | | | | | | | | | | |
| | | | | | | | | | | | | | | galactosidase | | | | | | | | | | | | | | |
| | | | | | | | | | | | | | | (galactostahdosis), PPGB | | | | | | | | | | | | | | |
| 40609 at | UNK AI47 | AI475497 | 6 | Pass | 10.50 | 3 45 | 10 | Pass | TRUE | FALSE | FALSE | 4 30 | 2.44 | | | | | | | | | | | | | | | |
| 38359_at | RASGRP2 | Y12336 | 6 | Pass | 54 67 | 8 16 | 13 | Pass | TRUE | FALSE | FALSE | 22 46 | 2.43 | RAS guanyl releasing | 1q13 | | | | | | | | | | | | | |
| | | | | | | | | | | | | | | protein 2 (calcium and | | | | | | | | | | | | | | |
| | | | | | | | | | | | | | | DAG-regulated), | | | | | | | | | | | | | | |
| | | | | | | | | | | | | | | RASGRP2 | | | | | | | | | | | | | | |
| 35807_at | CYBA | M21186 | 6 | Pass | 218.67 | 86 22 | 13 | Pass | TRUE | FALSE | FALSE | 89 92 | 2.43 | cytochrome b-245, alpha | 16q24 | | | | | | | | | | | | | |
| | | | | | | | | | | | | | | polypeptide, CYBA | | | | | | | | | | | | | | |
| 40875_s_at | SNRP70 | X06815 | 6 | Pass | 75.00 22 68 | 13 | Pass | TRUE | FALSE | FALSE | 30 85 | 2.43 | small nuclear | 19q13 3 | | | | | | | | | | | | | | |
| | | | | | | | | | | | | | | ribonucleoprotein 70kD | | | | | | | | | | | | | | |
| | | | | | | | | | | | | | | polypeptide (RNP | | | | | | | | | | | | | | |
| | | | | | | | | | | | | | | antigen), SNRP70 | | | | | | | | | | | | | | |
| 39711_at | PRKCSH | J03075 | 6 | Pass | 18.50 | 5 09 | 13 | Pass | TRUE | FALSE | FALSE | 7.62 | 2.43 | protein kinase C substrate | 19p13 1- | | | | | | | | | | | | | |
| | | | | | | | | | | | | | | 80K-H, PRKCS11 | p13 2 | | | | | | | | | | | | | |
| 39832_at | LOC51593 | AL096723 | 6 | Pass | 12 33 | 3 61 | 12 | Pass | TRUE | FALSE | FALSE | 5 08 | 2.43 | arsenate resistance protein | 7q21 | | | | | | | | | | | | | |
| | | | | | | | | | | | | | | ARS2, LOC51593 | | | | | | | | | | | | | | |
| 40160_at | DKFZP586 | AL080109 | 6 | Pass | 15.67 | 4.46 | 13 | Pass | TRUE | FALSE | FALSE | 6 46 | 2.42 | DKFZP586P2220 protein, | | | | | | | | | | | | | | |
| | | | | | | | | | | | | | | DKFZP586P2220 | | | | | | | | | | | | | | |
| 39823 at | H326 | U06631 | 5 | Pass | 20 40 | 5.32 | 12 | Pass | TRUE | FALSE | FALSE | 8 42 | 2.42 | H326, H326 | | | | | | | | | | | | | | |
| 37351 at | UP | X90858 | 6 | Pass | 21.00 | 11.92 | 12 | Pass | TRUE | FALSE | FALSE | 8 67 | 2.42 | undine phosphoylase, UP | 7 | | | | | | | | | | | | | |
| 35366 f at | UNK AF0 | AF015128 | 6 | Pass | 30 00 | 20.82 | 13 | Pass | TRUE | FALSE | FALSE | 12 38 | 2.42 | | | | | | | | | | | | | | | |
| 41532_at | ZNF151 | Y09723 | 6 | Pass | 8.17 | 2.40 | 8 | Pass | TRUE | FALSE | FALSE | 3 38 | 2.42 | zinc finger protein 151 | 1p36 2- | | | | | | | | | | | | | |
| | | | | | | | | | | | | | | (pHZ-67), ZNF151 | p36 1 | | | | | | | | | | | | | |
| 36545_s_at | UNK_AB0 | AB011114 | 6 | Pass | 11.17 | 3.31 | 13 | Pass | TRUE | FALSE | FALSE | 4 62 | 2.42 | KIAA0542 gene product, | 22q12 2 | | | | | | | | | | | | | |
| | | | | | | | | | | | | | | KIAA0542 | | | | | | | | | | | | | | |
| 38297_at | PITPNM | X98654 | 6 | Pass | 53 00 | 15 01 | 13 | Pass | TRUE | FALSE | FALSE | 21.92 | 2.42 | phosphasidylinositol | 11q13 | | | | | | | | | | | | | |
| | | | | | | | | | | | | | | transfer protein, membrane | | | | | | | | | | | | | | |
| | | | | | | | | | | | | | | associated, PITPNM | | | | | | | | | | | | | | |
| 37121 _at | NKG7 | S69115 | 6 | Pass | 95.67 | 55.68 | 13 | Pass | TRUE | FALSE | FALSE | 39.62 | 2.41 | natural killer cell group 7 | 19 | | | | | | | | | | | | | |
| | | | | | | | | | | | | | | sequence; NKG7 | | | | | | | | | | | | | | |
| 31816_at | GAA | X55079 | 6 | Pass | 18.00 | 9 32 | 11 | Pass | TRUE | FALSE | FALSE | 7.45 | 2.41 | glucosidase, alpha, acid | 17q25 2- | | | | | | | | | | | | | |
| | | | | | | | | | | | | | | (Pompe disease, glycogen | q25.3 | | | | | | | | | | | | | |
| | | | | | | | | | | | | | | storage disease type II), | | | | | | | | | | | | | | |
| | | | | | | | | | | | | | | GAA | | | | | | | | | | | | | | |
| 38029_at | MDU1 | 302939 | 6 | Pass | 19.67 | 5.92 | 13 | Pass | TRUE | FALSE | FALSE | 8.15 | 2.41 | solute carrier family 3 | 11q13 | | | | | | | | | | | | | |
| | | | | | | | | | | | | | | (activators of dibasic and | | | | | | | | | | | | | | |
| | | | | | | | | | | | | | | neutral amino acid | | | | | | | | | | | | | | |
| | | | | | | | | | | | | | | transport), member 2, | | | | | | | | | | | | | | |
| | | | | | | | | | | | | | | SLCA2 | | | | | | | | | | | | | | |
| 32550_r_at | CEBPA | Y11525 | 6 | Pass | 27 00 | 10 02 | 10 | Pass | TRUE | FALSE | FALSE | 11 20 | 2.41 | CCAAT/enhancer binding | 19q13 1 | | | | | | | | | | | | | |
| | | | | | | | | | | | | | | protein (C/EBP), alpha, | | | | | | | | | | | | | | |
| | | | | | | | | | | | | | | CEBPA | | | | | | | | | | | | | | |
| 33613 at | UNK AA8 | AA806239 | 5 | Pass | 8 60 | 1 52 | 7 | Pass | TRUE | FALSE | FALSE | 3 57 | 2.41 | | | | | | | | | | | | | | | |
| 40127_at | PMX1 | M95929 | 6 | Pass | 16 67 | 5 20 | 13 | Pass | TRUE | FALSE | FALSE | 6.92 | 2.41 | paired mesoderm homeo | 1q24 | | | | | | | | | | | | | |
| | | | | | | | | | | | | | | box 1; PMX1 | | | | | | | | | | | | | | |
| 32116_at | UNK_AB0 | AB002405 | 6 | Pass | 38 50 | 8 89 | 13 | Pass | TRUE | FALSE | FALSE | 16.00 | 2.41 | expressed in activated | 17q25 | | | | | | | | | | | | | |
| | | | | | | | | | | | | | | T/LAK lymphocytes, LAK | | | | | | | | | | | | | | |
| | | | | | | | | | | | | | | 4P | | | | | | | | | | | | | | |
| 41258_at | KIAA0618 | N29665 | 6 | Pass | 35.50 | 6 25 | 13 | Pass | TRUE | FALSE | FALSE | 14 77 | 2.40 | KIAA0618 gene | | | | | | | | | | | | | | |
| | | | | | | | | | | | | | | product, hypothetical | | | | | | | | | | | | | | |
| | | | | | | | | | | | | | | protein 1′FL110267, | | | | | | | | | | | | | | |
| | | | | | | | | | | | | | | FL110267, KIAA0681 | | | | | | | | | | | | | | |
| 558_at | KRT1 | M98776 | 5 | Pass | 12 20 | 5.26 | 13 | Pass | TRUE | FALSE | FALSE | 5.08 | 2.40 | keratin 1 (epidermolytic | 12q11- | | | | | | | | | | | | | |
| | | | | | | | | | | | | | | hyper keratosis), KRT1 | | | | | | | | | | | | | | |
| 40414_at | VARS2 | X59303 | 6 | Pass | 20 33 | 4.46 | 13 | Pass | TRUE | FALSE | FALSE | 8 46 | 2.40 | valyl-tRNA synthetase 2, | 6p21.3 | | | | | | | | | | | | | |
| | | | | | | | | | | | | | | VARS2 | | | | | | | | | | | | | | |
| 956 at | TUBB | X79535 | 6 | Pass | 23 00 | 7.13 | 7 | Pass | TRUE | FALSE | FALSE | 9.57 | 2.40 | | | | | | | | | | | | | | | |
| 37040_at | KIAA0088 | D42041 | 6 | Pass | 37.50 | 11 67 | 13 | Pass | TRUE | FALSE | FALSE | 15.62 | 2.40 | KIAA0088 protein, | | | | | | | | | | | | | | |
| | | | | | | | | | | | | | | KIAA0088 | | | | | | | | | | | | | | |
| 36161_at | ADTB2 | M34175 | 6 | Pass | 18 83 | 7.94 | 13 | Pass | TRUE | FALSE | FALSE | 7.85 | 2.40 | adaptor-related protein | 17q11 2- | | | | | | | | | | | | | |
| | | | | | | | | | | | | | | complex 2, beta 1 subunit, | q12 | | | | | | | | | | | | | |
| | | | | | | | | | | | | | | AP2B1 | | | | | | | | | | | | | | |
| 626_s_at | IF135 | L78833 | 5 | Pass | 5.40 | 4 04 | 8 | Pass | TRUE | FALSE | FALSE | 2.25 | 2.40 | interferon-induced protein | 17q21 | | | | | | | | | | | | | |
| | | | | | | | | | | | | | | 35, IF135 | | | | | | | | | | | | | | |
| 36314_at | FLT3LG | U04806 | 6 | Pass | 13.50 | 3 67 | 11 | Pass | TRUE | FALSE | FALSE | 5 64 | 2.40 | fins-related tyrosine kinase | 19q13 3 | | | | | | | | | | | | | |
| | | | | | | | | | | | | | | 3 ligand, FLT3LG | | | | | | | | | | | | | | |
| 36030_at | DKFZP586 | AL080214 | 6 | Pass | 21 00 | 6.13 | 13 | Pass | TRUE | FALSE | FALSE | 8 77 | 2.39 | DKTZP58612223 protein, | | | | | | | | | | | | | | |
| | | | | | | | | | | | | | | DKFZP58612223 | | | | | | | | | | | | | | |
| 33883_at | SPTAN1 | J05243 | 6 | Pass | 13 17 | 2.64 | 10 | Pass | TRUE | FALSE | FALSE | 5 50 | 2.39 | spectin, alpha, non- | 9q33-q34 | | | | | | | | | | | | | |
| | | | | | | | | | | | | | | erythrocyte 1 (alpha- | | | | | | | | | | | | | | |
| | | | | | | | | | | | | | | fodin), SPTAN1 | | | | | | | | | | | | | | |
| 33813_at | TNFRSFIB | AI813532 | 6 | Pass | 167.50 | 76 07 | 13 | Pass | TRUE | FALSE | FALSE | 70 08 | 2.39 | tuor necrosis factor | 1p36.3- | | | | | | | | | | | | | |
| | | | | | | | | | | | | | | receptor superfamily, | p36 2 | | | | | | | | | | | | | |
| | | | | | | | | | | | | | | member 1B; TNFRSFIB | | | | | | | | | | | | | | |
| 38483_at | HSA0191 | AJ011916 | 6 | Pass | 37.50 | 15 98 | 13 | Pass | TRUE | FALSE | FALSE | 15.69 | 2.39 | hypothetical protein; | 17q13 | | | | | | | | | | | | | |
| | | | | | | | | | | | | | | HSA019916 | | | | | | | | | | | | | | |
| 33412_at | LGALS1 | AI535946 | 6 | Pass | 99 50 | 46 06 | 13 | Pass | TRUE | FALSE | FALSE | 41.77 | 2.38 | lectin, galactoside-binding, | 22q13 1 | | | | | | | | | | | | | |
| | | | | | | | | | | | | | | soluble, 1 (galectin 1), | | | | | | | | | | | | | | |
| | | | | | | | | | | | | | | LGALS1 | | | | | | | | | | | | | | |
| 34749_at | SLC31A2 | U83461 | 5 | Pass | 18 40 | 8.65 | 11 | Pass | TRUE | FALSE | FALSE | 7.73 | 2.38 | solute carrier family 31 | 9q31-q32 | | | | | | | | | | | | | |
| | | | | | | | | | | | | | | (copper transporters), | | | | | | | | | | | | | | |
| | | | | | | | | | | | | | | member 2, SLC31A2 | | | | | | | | | | | | | | |
| 32660_at | UNK_AB0 | AB002340 | 6 | Pass | 16 67 | 4 23 | 10 | Pass | TRUE | FALSE | FALSE | 7.00 | 2.38 | KIAA0342 gene product, | | | | | | | | | | | | | | |
| | | | | | | | | | | | | | | KIAA0342 | | | | | | | | | | | | | | |
| 905_at | GUK1 | L76200 | 6 | Pass | 46 33 | 17 22 | 13 | Pass | TRUE | FALSE | FALSE | 19.46 | 2.38 | guanylate kinase 1, GUK1 | 1q32-141 | | | | | | | | | | | | | |
| 277_at | MCL1 | L08246 | 6 | Pass | 115 17 | 44 97 | 13 | Pass | TRUE | FALSE | FALSE | 48.38 | 2.38 | myeloid cell leukemia | 1q21 | | | | | | | | | | | | | |
| | | | | | | | | | | | | | | sequence 1 (BCL2- | | | | | | | | | | | | | | |
| | | | | | | | | | | | | | | related), MCL1 | | | | | | | | | | | | | | |
| 922_at | PPP2R1A | J02902 | 6 | Pass | 33.50 | 8 36 | 13 | Pass | TRUE | FALSE | FALSE | 14 08 | 2.38 | protein phosphatase 2 | | | | | | | | | | | | | | |
| | | | | | | | | | | | | | | (formerly 2A), regulatory | | | | | | | | | | | | | | |
| | | | | | | | | | | | | | | subunit A (PR 65), alpha | | | | | | | | | | | | | | |
| | | | | | | | | | | | | | | isoform, PPP2R1A | | | | | | | | | | | | | | |
| 33433_at | DOK1 | AF035299 | 6 | Pass | 20.33 | 17.93 | 11 | Pass | TRUE | FALSE | FALSE | 8.55 | 2.38 | docking protein 1, 62kD | 2p13 | | | | | | | | | | | | | |
| | | | | | | | | | | | | | | (downstream of tyrosine | | | | | | | | | | | | | | |
| | | | | | | | | | | | | | | kinase 1), DOK1 | | | | | | | | | | | | | | |
| 1403_s_at | SCYA5 | M21121 | 6 | Pass | 181.00 | 62.86 | 13 | Pass | TRUE | FALSE | FALSE | 76.15 | 2.38 | small inducible cytokine | 17q11 2- | | | | | | | | | | | | | |
| | | | | | | | | | | | | | | A5 (RANTES), SCYA5 | q12 | | | | | | | | | | | | | |
| 37026_at | COPEB | AF001461 | 6 | Pass | 26.50 | 13.94 | 13 | Pass | TRUE | FALSE | FALSE | 11.15 | 2.38 | core promoter element | 10p15 | | | | | | | | | | | | | |
| | | | | | | | | | | | | | | binding protein; COPEB | | | | | | | | | | | | | | |
| 1997_s_at | BAX | U19599 | 6 | Pass | 11.83 | 5 04 | 11 | Pass | TRUE | FALSE | FALSE | 5.00 | 2.37 | BCL2-associates X | 19q13 3- | | | | | | | | | | | | | |
| | | | | | | | | | | | | | | protein; BAX | | | | | | | | | | | | | | |
| 33866 at | TPM4 | X05276 | 6 | Pass | 7 33 | 1 63 | 10 | Pass | TRUE | FALSE | FALSE | 3.10 | 2.37 | tropomyosin 4; TPM4 | 19p13 1 | | | | | | | | | | | | | |
| 38051_at | MAL | X76220 | 6 | Pass | 31 83 | 15 48 | 13 | Pass | TRUE | FALSE | FALSE | 13.46 | 2.36 | mal, T-cell differentiation | 2cen-q13 | | | | | | | | | | | | | |
| | | | | | | | | | | | | | | protein, MAL | | | | | | | | | | | | | | |
| 1347_at | CDC25B | S78187 | 6 | Pass | 33 83 | 10.44 | 13 | Pass | TRUE | FALSE | FALSE | 14 31 | 2.36 | cell division cycle 25B, | 20p13 | | | | | | | | | | | | | |
| | | | | | | | | | | | | | | CDC25B | | | | | | | | | | | | | | |
| 36603_at | GCNIL1 | D86973 | 6 | Pass | 14.00 | 2 61 | 13 | Pass | TRUE | FALSE | FALSE | 5.92 | 2.36 | GCN1 (general control of | 12q24 2 | | | | | | | | | | | | | |
| | | | | | | | | | | | | | | amino-acid synthesis 1, | | | | | | | | | | | | | | |
| | | | | | | | | | | | | | | yeast)-like 1, GCNIL1 | | | | | | | | | | | | | | |
| 36058_at | DKFZP586 | AL096741 | 6 | Pass | 14.33 | 2 42 | 13 | Pass | TRUE | FALSE | FALSE | 6.08 | 2.36 | hypothetical protein, | 22 | | | | | | | | | | | | | |
| | | | | | | | | | | | | | | DKFZP586O223 | | | | | | | | | | | | | | |
| 33361_at | GNG3LG | AF052149 | 6 | Pass | 14 67 | 2 66 | 13 | Pass | TRUE | FALSE | FALSE | 6 23 | 2.35 | guanine nucleotide binding | 11q12- | | | | | | | | | | | | | |
| | | | | | | | | | | | | | | protein (G protein), | q13 5 | | | | | | | | | | | | | |
| | | | | | | | | | | | | | | gamma 3, linked. | | | | | | | | | | | | | | |
| 34491_at | OASL | AJ225089 | 6 | Pass | 7 33 | 5 39 | 8 | Pass | TRUE | FALSE | FALSE | 3.13 | 2.35 | 2′-5′oligoadcylate | 12q24 2 | | | | | | | | | | | | | |
| | | | | | | | | | | | | | | synthetase-like, OASL | | | | | | | | | | | | | | |
| 41165_g_at | IGHM | X67301 | 6 | Pass | 150 00 | 56 88 | 13 | Pass | TRUE | FALSE | FALSE | 63 92 | 2.35 | immunoglobulin heavy | 14q32 33 | | | | | | | | | | | | | |
| | | | | | | | | | | | | | | constant mu, IGHM | | | | | | | | | | | | | | |
| 31504_at | HDLBP | M64098 | 6 | Pass | 13 67 | 4.68 | 12 | Pass | TRUE | FALSE | FALSE | 5.83 | 2.34 | high density lipoprotein 2q37 | | | | | | | | | | | | | | |
| | | | | | | | | | | | | | | binding protein (vigiun), | | | | | | | | | | | | | | |
| | | | | | | | | | | | | | | HDLBP | | | | | | | | | | | | | | |
| 1237_at | IER3 | S81914 | 5 | Pass | 10.40 | 5 32 | 9 | Pass | TRUE | FALSE | FALSE | 4 44 | 2.34 | immediate early response | 6p21.3 | | | | | | | | | | | | | |
| | | | | | | | | | | | | | | 3, IER3 | | | | | | | | | | | | | | |
| 38117_at | SEC24C | D38555 | 6 | Pass | 15.67 | 3 44 | 10 | Pass | TRUE | FALSE | FALSE | 6.70 | 2.34 | SEC24 (S. cerevisiae) | 10 | | | | | | | | | | | | | |
| | | | | | | | | | | | | | | related gene family, | | | | | | | | | | | | | | |
| | | | | | | | | | | | | | | member C, SEC24C | | | | | | | | | | | | | | |
| 38423 at | UNK L38 | L38935 | 6 | Pass | 36 50 | 12 47 | 13 | Pass | TRUE | FALSE | FALSE | 15 62 | 2.34 | | | | | | | | | | | | | | | |
| 38064_at | LRP | X79882 | 6 | Pass | 23 17 | 10 70 | 13 | Pass | TRUE | FALSE | FALSE | 9.92 | 2.33 | major protein, MVP | 16p13 1- | | | | | | | | | | | | | |
| | | | | | | | | | | | | | | | p11 2 | | | | | | | | | | | | | |
| 39158_at | ATF5 | AB021663 | 6 | Pass | 7 00 | 3.58 | 10 | Pass | TRUE | FALSE | FALSE | 3.00 | 2.33 | activating transcription | | | | | | | | | | | | | | |
| | | | | | | | | | | | | | | factor 5, ATF5 | | | | | | | | | | | | | | |
| 39507_at | KIAA0843 | AB020650 | 6 | Pass | 10.50 | 4.97 | 12 | Pass | TRUE | FALSE | FALSE | 4 50 | 2.33 | KIAA0843 protein, | | | | | | | | | | | | | | |
| | | | | | | | | | | | | | | KIAA0843 | | | | | | | | | | | | | | |
| 34695_at | GA17 | AI816724 | 6 | Pass | 30.67 | 18.23 | 13 | Pass | TRUE | FALSE | FALSE | 13 15 | 2.33 | SWI/SNF related, matrix | 17q23- | | | | | | | | | | | | | |
| | | | | | | | | | | | | | | associated, actin dependent | q24 | | | | | | | | | | | | | |
| | | | | | | | | | | | | | | regulated of chromatin, | | | | | | | | | | | | | | |
| | | | | | | | | | | | | | | subfamily d, member 2, | | | | | | | | | | | | | | |
| | | | | | | | | | | | | | | SMARCD2 | | | | | | | | | | | | | | |
| 39904_at | CCR1 | D10925 | 5 | Pass | 17.60 | 14 91 | 9 | Pass | TRUE | FALSE | FALSE | 7.56 | 2.33 | chemokine (C—C motif) | 3p21 | | | | | | | | | | | | | |
| | | | | | | | | | | | | | | receptor 1, CCR1 | | | | | | | | | | | | | | |
| 37799_{—l at} | ASGR2 | X55284 | 6 | Pass | 13 33 | 6 31 | 11 | Pass | TRUE | FALSE | FALSE | 5 73 | 2.33 | astaloglycoprotein receptor | 17p | | | | | | | | | | | | | |
| | | | | | | | | | | | | | | 2, ASGR2 | | | | | | | | | | | | | | |
| 496_s_at | IL11RA | U32324 | 6 | Pass | 9 67 | 3 20 | 13 | Pass | TRUE | FALSE | FALSE | 4.15 | 2.33 | interleukin 11 receptor, | 9p13 | | | | | | | | | | | | | |
| | | | | | | | | | | | | | | alpha, IL11RA | | | | | | | | | | | | | | |
| 849_g_at | UNK_U19 | U19261 | 6 | Pass | 10.00 | 2.45 | 13 | Pass | TRUE | FALSE | FALSE | 4.31 | 2.32 | TNF receptor-associated | 9q33-q34 | | | | | | | | | | | | | |
| | | | | | | | | | | | | | | factor 1, TRAF1 | | | | | | | | | | | | | | |
| 34691_f_at | ARPC4 | AF006087 | 6 | Pass | 25.00 | 6 63 | 13 | Pass | TRUE | FALSE | FALSE | 10.77 | 2.32 | actin related protein 2/3 | | | | | | | | | | | | | | |
| | | | | | | | | | | | | | | complex, subunit 4 (20 | | | | | | | | | | | | | | |
| | | | | | | | | | | | | | | kD), ARPC4 | | | | | | | | | | | | | | |
| 35244_at | KIAA0460 | AB007929 | 5 | Pass | 11.60 | 3 65 | 11 | Pass | TRUE | FALSE | FALSE | 5 00 | 2.32 | KIAA0460 protein, | 1 | | | | | | | | | | | | | |
| | | | | | | | | | | | | | | KIAA0460 | | | | | | | | | | | | | | |
| 32336_at | ALDOA | X05236 | 6 | Pass | 129.33 | 37.93 | 13 | Pass | TRUE | FALSE | FALSE | 55.77 | 2.32 | aldalase A. fructose- | 16q22- | | | | | | | | | | | | | |
| | | | | | | | | | | | | | | bisphospate, ALDOA | q24 | | | | | | | | | | | | | |
| 33838_at | D6S52E | M33519 | 6 | Pass | 30.67 | 4 37 | 13 | Pass | TRUE | FALSE | FALSE | 13 23 | 2.32 | HLA-B associated | 6p21 3 | | | | | | | | | | | | | |
| | | | | | | | | | | | | | | transcript-3, D6S52E | | | | | | | | | | | | | | |
| 40885_s_at | UNK_N30 | N30151 | 6 | Pass | 18 33 | 2.58 | 13 | Pass | TRUE | FALSE | FALSE | 7.92 | 2.31 | eukaryotic translation | | | | | | | | | | | | | | |
| | | | | | | | | | | | | | | immitation factor 4B, EIF4B | | | | | | | | | | | | | | |
| 38893_at | NCF4 | AL008637 | 6 Pass | 55.17 | 27.67 | 13 | Pass | TRUE | FALSE | FALSE | 23.85 | 2.31 | neutrophil cytosolic factor | 22q13 1 | | | | | | | | | | | | | | |
| | | | | | | | | | | | | | | 4 (40kD), NCF4 | | | | | | | | | | | | | | |
| 40365_at | GNA15 | M63904 | 6 | Pass | 15.83 | 7.47 | 13 | Pass | TRUE | FALSE | FALSE | 6.85 | 2.31 | guanine nucleotide binding | 19p13.3 | | | | | | | | | | | | | |
| | | | | | | | | | | | | | | protein (G protein), alpha | | | | | | | | | | | | | | |
| | | | | | | | | | | | | | | 15 (Gq class); GNA15 | | | | | | | | | | | | | | |
| 39795_at | CLAPM1 | D63475 | 6 | Pass | 80 00 | 25 55 | 13 | Pass | TRUE | FALSE | FALSE | 34.62 | 2.31 | adaptor-related protein | 3q28 | | | | | | | | | | | | | |
| | | | | | | | | | | | | | | complex 2, mu 1 subunit, | | | | | | | | | | | | | | |
| | | | | | | | | | | | | | | AP2M1 | | | | | | | | | | | | | | |
| 38729_at | FKBP4 | M88279 | 6 | Pass | 11 17 | 3 06 | 12 | Pass | TRUE | FALSE | FALSE | 4 83 | 2.31 | FK506-binding protein 4 | | | | | | | | | | | | | | |
| | | | | | | | | | | | | | | (59kD), FKBP4 | | | | | | | | | | | | | | |
| 39385_at | ANPEP | M22324 | 6 | Pass | 16 50 | 5.75 | 7 | Pass | TRUE | FALSE | FALSE | 7 14 | 2.31 | alanyl (membrane) | 15q25- | | | | | | | | | | | | | |
| | | | | | | | | | | | | | | aminopeptidase | q26 | | | | | | | | | | | | | |
| | | | | | | | | | | | | | | (aminopeptidase N, | | | | | | | | | | | | | | |
| | | | | | | | | | | | | | | aminopeptidase M, | | | | | | | | | | | | | | |
| | | | | | | | | | | | | | | microsomal | | | | | | | | | | | | | | |
| | | | | | | | | | | | | | | aminopeptidase, CD13, | | | | | | | | | | | | | | |
| | | | | | | | | | | | | | | p150), ANPEP | | | | | | | | | | | | | | |
| 37298_at | GABARA | AF044671 | 6 | Pass | 123 67 | 48 80 | 13 | Pass | TRUE | FALSE | FALSE | 53 54 | 2.31 | GABA(A) receptor- | 17 | | | | | | | | | | | | | |
| | | | | | | | | | | | | | | asociated protein, | | | | | | | | | | | | | | |
| | | | | | | | | | | | | | | GABARAP | | | | | | | | | | | | | | |
| 2019 s at | ITGB7 | M68892 | 6 | Pass | 13 50 | 6 35 | 13 | Pass | TRUE | FALSE | FALSE | 5.85 | 2.31 | integrin, beta 7, ITGB7 | 12q13 13 | | | | | | | | | | | | | |
| 36035_at | GPAA1 | AB802135 | 6 | Pass | 24 33 | 6 83 | 13 | Pass | TRUE | FALSE | FALSE | 10 54 | 2.31 | glycophosphatidylinositol | 8q24 3 | | | | | | | | | | | | | |
| | | | | | | | | | | | | | | anchor attachment 1, | | | | | | | | | | | | | | |
| | | | | | | | | | | | | | | GPAA1 | | | | | | | | | | | | | | |
| 37796_at | UNK_AF0 | AF053356 | 6 | Pass | 28 33 | 2 94 | 11 | Pass | TRUE | FALSE | FALSE | 12.27 | 2.31 | leucine-rich neuronal | 7q22 | | | | | | | | | | | | | |
| | | | | | | | | | | | | | | protein, LRN | | | | | | | | | | | | | | |
| 32174_at | SLC9A3R | AF015926 | 6 | Pass | 29.83 | 5 27 | 13 | Pass | TRUE | FALSE | FALSE | 12 92 | 2.31 | solute carries family 9 | | | | | | | | | | | | | | |
| | | | | | | | | | | | | | | (sodium/hydrogen | | | | | | | | | | | | | | |
| | | | | | | | | | | | | | | exchanger), isoform 3 | | | | | | | | | | | | | | |
| | | | | | | | | | | | | | | regulatory factor 1, | | | | | | | | | | | | | | |
| | | | | | | | | | | | | | | SLC9A3R1 | | | | | | | | | | | | | | |
| 36322_at | FUT7 | AB012668 | 4 | Pass | 10.00 | 3.46 | 9 | Pass | TRUE | FALSE | FALSE | 4.33 | 2.31 | fucosyltransferase 7 (alpha | 9 | | | | | | | | | | | | | |
| | | | | | | | | | | | | | | (1,3) fucosyltransferase), | | | | | | | | | | | | | | |
| | | | | | | | | | | | | | | FUT7 | | | | | | | | | | | | | | |
| 38091_at | LGALS9 | Z49107 | 6 | Pass | 60.00 | 23 87 | 13 | Pass | TRUE | FALSE | FALSE | 26 00 | 2.31 | lectin, galactoside-binding, | | | | | | | | | | | | | | |
| | | | | | | | | | | | | | | soluble, 9 (galectin 9), | | | | | | | | | | | | | | |
| | | | | | | | | | | | | | | LGALS9 | | | | | | | | | | | | | | |
| 33706_at | SART1 | AB006198 | 6 | Pass | 19 50 | 4.72 | 13 | Pass | TRUE | FALSE | FALSE | 8 46 | 2.30 | squamous cell carcinoma | | | | | | | | | | | | | | |
| | | | | | | | | | | | | | | antigen recognised by 7 | | | | | | | | | | | | | | |
| | | | | | | | | | | | | | | cells, SART1 | | | | | | | | | | | | | | |
| 1919 at | VAV1 | X16316 | 4 | Pass | 13 25 | 3.77 | 8 | Pass | TRUE | FALSE | FALSE | 5 75 | 2.30 | vav 1 oncogene, VAV1 | 19p13 2 | | | | | | | | | | | | | |
| 39053_at | HPRP3P | AF016370 | 6 | Pass | 6.33 | 1.63 | 8 | Pass | TRUE | FALSE | FALSE | 2.75 | 2.30 | U4/U6-associated RNA | 1q21 1 | | | | | | | | | | | | | |
| | | | | | | | | | | | | | | splicing factor, HPRP3P | | | | | | | | | | | | | | |
| 880_at | FKBPIA | M34539 | 6 | Pass | 59 33 | 20 33 | 13 | Pass | TRUE | FALSE | FALSE | 25.77 | 2.30 | FK506-binding proten 1A | 20p13 | | | | | | | | | | | | | |
| | | | | | | | | | | | | | | (12kD), FKBPIA | | | | | | | | | | | | | | |
| 37947_at | KIAA0043 | D26362 | 6 | Pass | 16 50 | 8.46 | 12 | Pass | TRUE | FALSE | FALSE | 7.17 | 2.30 | KIAA043 gene product, | | | | | | | | | | | | | | |
| | | | | | | | | | | | | | | KIAA0043 | | | | | | | | | | | | | | |
| 37376 at | LOC51035 | M68864 | 6 | Pass | 28 33 | 9 73 | 13 | Pass | TRUE | FALSE | FALSE | 12 31 | 2.30 | ORF; LOC51035 | | | | | | | | | | | | | | |
| 32218 at | UNK AF0 | AF034176 | 6 | Pass | 31 67 | 11.67 | 13 | Pass | TRUE | FALSE | FALSE | 13 77 | 2.30 | | | | | | | | | | | | | | | |
| 41460_at | SIP | AF080561 | 6 | Pass | 9 00 | 3 95 | 12 | Pass | TRUE | FALSE | FALSE | 3.92 | 2.30 | SY1 interacting protein, | | | | | | | | | | | | | | |
| | | | | | | | | | | | | | | SIP | | | | | | | | | | | | | | |
| 35749_at | TADA3L | AF069733 | 6 | Pass | 44 00 | 13 48 | 13 | Pass | TRUE | FALSE | FALSE | 19.15 | 2.30 | transcriptional adaptor 3 | | | | | | | | | | | | | | |
| | | | | | | | | | | | | | | (ADA3, yeast homolog)- | | | | | | | | | | | | | | |
| | | | | | | | | | | | | | | like (PCAF histone | | | | | | | | | | | | | | |
| | | | | | | | | | | | | | | acctylase complex), | | | | | | | | | | | | | | |
| | | | | | | | | | | | | | | TADA3L | | | | | | | | | | | | | | |
| 38710_at | UNK_AL0 | AL096714 | 6 | Pass | 29.67 | 8 82 | 13 | Pass | TRUE | FALSE | FALSE | 12.92 | 2.30 | hypothetical protein | | | | | | | | | | | | | | |
| | | | | | | | | | | | | | | FLJ20113, FLJ20113 | | | | | | | | | | | | | | |
| 41386_r_at | KIAA0346 | AB802344 | 6 | Pass | 16.83 | 5.60 | 12 | Pass | TRUE | FALSE | FALSE | 7 33 | 2.30 | KIAA0346 protein, | 17p13 1 | | | | | | | | | | | | | |
| | | | | | | | | | | | | | | KIAA0346 | | | | | | | | | | | | | | |
| 344 s at | CNP | D13146 | 5 | Pass | 22.60 | 8.26 | 13 | Pass | TRUE | FALSE | FALSE | 9 85 | 2.30 | | | | | | | | | | | | | | | |
| 36162 at | BSG | X64364 | 6 | Pass | 18 00 | 2 28 | 13 | Pass | TRUE | FALSE | FALSE | 7 85 | 2.29 | basigin, BSG | 19p13 3 | | | | | | | | | | | | | |
| 36937_s_at | CLIM1 | U90878 | 6 | Pass | 18.17 | 6.40 | 13 | Pass | TRUE | FALSE | FALSE | 7 92 | 2.29 | carboxy terminal LIM | 10qter | | | | | | | | | | | | | |
| | | | | | | | | | | | | | | domain protein 1, CLIM1 | | | | | | | | | | | | | | |
| 1158_s_at | CALM3 | J04046 | 6 | Pass | 22.50 | 4.04 | 11 | Pass | TRUE | FALSE | FALSE | 9.82 | 2.29 | calmodulin 3 | 19q13.2- | | | | | | | | | | | | | |
| | | | | | | | | | | | | | | (phosphorylase kinase, | q13.3 | | | | | | | | | | | | | |
| | | | | | | | | | | | | | | delta), CALM3 | | | | | | | | | | | | | | |
| 38976 at | CORO1A | D44497 | 6 | Pass | 230 67 | 59.84 | 13 | Pass | TRUE | FALSE | FALSE | 100.77 | 2.29 | coronim, actin-binding | | | | | | | | | | | | | | |
| | | | | | | | | | | | | | | protein, 1A, CORO1A | | | | | | | | | | | | | | |
| 39722_at | NCOR1 | AF044209 | 6 | Pass | 17 17 | 8 80 | 10 | Pass | TRUE | FALSE | FALSE | 7 50 | 2.29 | nuclear receptor co- | 17p11 2 | | | | | | | | | | | | | |
| | | | | | | | | | | | | | | repressor 1, NCOR1 | | | | | | | | | | | | | | |
| 32909 at | AQP5 | U46569 | 6 | Pass | 11 67 | 3 50 | 10 | Pass | TRUE | FALSE | FALSE | 5 10 | 2.29 | aquaporin 5; AQP5 | 12q13 | | | | | | | | | | | | | |
| 932_r_at | 7NF91 | L11672 | 6 | Pass | 19 17 | 9.22 | 13 | Pass | TRUE | FALSE | FALSE | 8.38 | 2.29 | zinc finger protein 91 | 19p13 1- | | | | | | | | | | | | | |
| | | | | | | | | | | | | | | (HPF7, HTF10), ZNF91 | p12 | | | | | | | | | | | | | |
| 39908_at | PAF65A | AF069735 | 6 | Pass | 111 50 | 49 03 | 9 | Pass | TRUE | FALSE | FALSE | 48.78 | 2.29 | PCAF associated factor 65 | | | | | | | | | | | | | | |
| | | | | | | | | | | | | | | alpha, PAF65A | | | | | | | | | | | | | | |
| 31638 at | NDUFS7 | AC005329 | 6 | Pass | 20 00 | 8.15 | 8 | Pass | TRUE | FALSE | FALSE | 8 75 | 2.29 | | | | | | | | | | | | | | | |
| 33137_at | LTBP4 | Y13622 | 6 | Pass | 12 00 | 2.90 | 12 | Pass | TRUE | FALSE | FALSE | 5 25 | 2.29 | Intent transforming growth | 19q13 1- | | | | | | | | | | | | | |
| | | | | | | | | | | | | | | factor beta binding protein | q13 2 | | | | | | | | | | | | | |
| | | | | | | | | | | | | | | 4, LTBP4 | | | | | | | | | | | | | | |
| 39343_at | HSU53209 | AW026656 | 6 | Pass | 7 17 | 4 12 | 7 | Pass | TRUE | FALSE | FALSE | 3 14 | 2.28 | transformer-2-alpha (hta-2 | | | | | | | | | | | | | | |
| | | | | | | | | | | | | | | alpha); HSU53209 | | | | | | | | | | | | | | |
| 32370_at | GZMB | M57888 | 6 | Pass | 46 83 | 29.69 | 13 | Pass | TRUE | FALSE | FALSE | 20.54 | 2.28 | gianzyme B (gianzyme 2, | 14q11 2 | | | | | | | | | | | | | |
| | | | | | | | | | | | | | | cytoxic T-lymphocyte- | | | | | | | | | | | | | | |
| | | | | | | | | | | | | | | associated serine esterase | | | | | | | | | | | | | | |
| | | | | | | | | | | | | | | 1) GZMB | | | | | | | | | | | | | | |
| 816_g_at | DOK1 | U70987 | 6 | Pass | 30 17 | 18 29 | 13 | Pass | TRUE | FALSE | FALSE | 13 23 | 2.28 | docking protein 1, 62kD | 2p13 | | | | | | | | | | | | | |
| | | | | | | | | | | | | | | (downstream of tyrosine | | | | | | | | | | | | | | |
| | | | | | | | | | | | | | | kinase 1), DOK1 | | | | | | | | | | | | | | |
| 32658 at | UNK AL0 | AL031228 | 6 | Pass | 10 33 | 2 25 | 13 | Pass | TRUE | FALSE | FALSE | 4 54 | 2.28 | | | | | | | | | | | | | | | |
| 36208_at | FSRG1 | D42040 | 6 | Pass | 20 83 | 4 36 | 13 | Pass | TRUE | FALSE | FALSE | 9 15 | 2.28 | bromodomain-containing | 6p21.3 | | | | | | | | | | | | | |
| | | | | | | | | | | | | | | 2, BRD2 | | | | | | | | | | | | | | |
| 38998 g at | SLC25A1 | X96924 | 6 | Pass | 11 83 | 3.82 | 10 | Pass | TRUE | FALSE | FALSE | 5.20 | 2.28 | solute cancer family 25 | 22q11 21 | | | | | | | | | | | | | |
| | | | | | | | | | | | | | | (mitochondrial carrier, | | | | | | | | | | | | | | |
| | | | | | | | | | | | | | | citrate transporter), | | | | | | | | | | | | | | |
| | | | | | | | | | | | | | | member 1 SLC25A1 | | | | | | | | | | | | | | |
| 33146_at | MCL1 | L08246 | 6 | Pass | 116 17 | 36.29 | 13 | Pass | TRUE | FALSE | FALSE | 51 08 | 2.27 | mycloid cell leukemia | 1q21 | | | | | | | | | | | | | |
| | | | | | | | | | | | | | | seqaence 1 (BCL2- | | | | | | | | | | | | | | |
| | | | | | | | | | | | | | | related), MCL1 | | | | | | | | | | | | | | |
| 402_s_at | ICAM3 | X69819 | 6 | Pass | 46 33 | 18.16 | 13 | Pass | TRUE | FALSE | FALSE | 20 38 | 2.27 | intercellular adhesion | 19q13 3- | | | | | | | | | | | | | |
| | | | | | | | | | | | | | | molecule 3, ICAM3 | p13 2 | | | | | | | | | | | | | |
| 39971_at | LYL1 | M22637 | 6 | Pass | 13.83 | 3.54 | 11 | Pass | TRUE | FALSE | FALSE | 6.09 | 2.27 | lymphoblastic leukemia | 19p13 2 | | | | | | | | | | | | | |
| | | | | | | | | | | | | | | derived sequence 1, LYL1 | | | | | | | | | | | | | | |
| 32646_at | KIAA0449 | AB007918 | 5 | Pass | 22 20 | 3 56 | 9 | Pass | TRUE | FALSE | FALSE | 9.78 | 2.27 | KIAA0446 protein, | 1 | | | | | | | | | | | | | |
| | | | | | | | | | | | | | | KIAA0449 | | | | | | | | | | | | | | |
| 31901_at | KCNAB2 | AF044253 | 6 | Pass | 28.50 | 8 12 | 9 | Pass | TRUE | FALSE | FALSE | 12.56 | 2.27 | potassium voltage-gated | 1p36 3 | | | | | | | | | | | | | |
| | | | | | | | | | | | | | | channel, shaken-related | | | | | | | | | | | | | | |
| | | | | | | | | | | | | | | subfamily, beta member 2, | | | | | | | | | | | | | | |
| | | | | | | | | | | | | | | KCNAB2 | | | | | | | | | | | | | | |
| 40147_at | VAT1 | U18009 | 6 | Pass | 14 83 | 5 74 | 13 | Pass | TRUE | FALSE | FALSE | 6.54 | 2.27 | membrane protein of | 17q21 | | | | | | | | | | | | | |
| | | | | | | | | | | | | | | cholinergic sympatic | | | | | | | | | | | | | | |
| | | | | | | | | | | | | | | vesicles, VAT1 | | | | | | | | | | | | | | |
| 40130_at | FSTL1 | U068636 | Pass | 17.00 | 5 66 | 12 | Pass | TRUE | FALSE | FALSE | 7 50 | 2.27 | folistatin-like 1, FSTL1 | 7q21 2- | | | | | | | | | | | | | | |
| | | | | | | | | | | | | | | | q31 1 | | | | | | | | | | | | | |
| 151_s_at | UNK_V00 | V00599 | 6 | Pass | 42 17 | 11 99 | 13 | Pass | TRUE | FALSE | FALSE | 18.62 | 2.27 | tubulin, beta polypeptide, | 6p21.3 | | | | | | | | | | | | | |
| | | | | | | | | | | | | | | TUBB | | | | | | | | | | | | | | |
| 766_at | LGALS9 | AB006782 | 6 | Pass | 31 33 | 14.72 | 13 | Pass | TRUE | FALSE | FALSE | 13.85 | 2.26 | lectin, galactoside-binding, | | | | | | | | | | | | | | |
| | | | | | | | | | | | | | | soluble, 9 (galactin 9), | | | | | | | | | | | | | | |
| | | | | | | | | | | | | | | LGALS9 | | | | | | | | | | | | | | |
| 33863_at | ORP150 | U65785 | 6 | Pass | 25.50 | 5.61 | 11 | Pass | TRUE | FALSE | FALSE | 11.27 | 2.26 | oxygen regulated protein | 11 | | | | | | | | | | | | | |
| | | | | | | | | | | | | | | (150kD), ORP150 | | | | | | | | | | | | | | |
| 41728_at | KIAA0152 | D63486 | 6 | Pass | 19 83 | 5.00 | 13 | Pass | TRUE | FALSE | FALSE | 8 77 | 2.26 | KIAA0152 gene product; | 12 | | | | | | | | | | | | | |
| | | | | | | | | | | | | | | KIAA0152 | | | | | | | | | | | | | | |
| 37307_at | GNA12 | X04828 | 6 | Pass | 14767 | 57.65 | 13 | Pass | TRUE | FALSE | FALSE | 65 31 | 2.26 | guanine nucleotide binding | 3p21 | | | | | | | | | | | | | |
| | | | | | | | | | | | | | | protein (G protein), alpha | | | | | | | | | | | | | | |
| | | | | | | | | | | | | | | inhibiting activity | | | | | | | | | | | | | | |
| | | | | | | | | | | | | | | polypeptide 2, GNA12 | | | | | | | | | | | | | | |
| 33826_at | CIZ1 | AL120500 | 6 | Pass | 21.83 | 3.97 | 12 | Pass | TRUE | FALSE | FALSE | 9.67 | 2.26 | cip1-interacting zinc | 9q34.1 | | | | | | | | | | | | | |
| | | | | | | | | | | | | | | finger protein: CIZ1 | | | | | | | | | | | | | | |
| 1795 g at | CCND3 | M92287 | 6 | Pass | 94.50 | 24 91 | 13 | Pass | TRUE | FALSE | FALSE | 41.85 | 2.26 | cyclin D3, CCND3 | 6p21 | | | | | | | | | | | | | |
| 38674_at | IGFBP6 | AA115140 | 6 | Pass | 103.50 | 49 49 | 13 | Pass | TRUE | FALSE | FALSE | 45 85 | 2.26 | hypothetical protein | 9 | | | | | | | | | | | | | |
| | | | | | | | | | | | | | | FLJ110262, FLJ10262 | | | | | | | | | | | | | | |
| 39423_f_at | SPOP | AJ000644 | 6 | Pass | 12.50 | 7.74 | 11 | Pass | TRUE | FALSE | FALSE | 5.55 | 2.25 | speckle-type POZ protein, | | | | | | | | | | | | | | |
| | | | | | | | | | | | | | | SPOP | | | | | | | | | | | | | | |
| 39133_at | GCN5L1 | AI525379 | 5 | Pass | 7.00 | 2 00 | 9 | Pass | TRUE | FALSE | FALSE | 3 11 | 2.25 | GCN5 (general control of | 12q13- | | | | | | | | | | | | | |
| | | | | | | | | | | | | | | amino-acid synthesis, | q14 | | | | | | | | | | | | | |
| | | | | | | | | | | | | | | yeast, homolog)-like 1, | | | | | | | | | | | | | | |
| | | | | | | | | | | | | | | GCN5L1 | | | | | | | | | | | | | | |
| 34224 at | KIAA0954 | AC004770 | 6 | Pass | 4.67 | 3.33 | 13 | Pass | TRUE | FALSE | FALSE | 2 08 | 2.25 | | | | | | | | | | | | | | | |
| 32800_at | RXRA | U66306 | 6 | Pass | 56.33 | 25 13 | 13 | Pass | TRUE | FALSE | FALSE | 25 08 | 2.25 | retinoid X receptor, alpha, | 9q34 3 | | | | | | | | | | | | | |
| | | | | | | | | | | | | | | RXRA | | | | | | | | | | | | | | |
| 33232_at | CRIP1 | AI017574 | 6 | Pass | 73.33 | 19 72 | 13 | Pass | TRUE | FALSE | FALSE | 32 69 | 2.24 | cysteine-rich protein 1 | 7q11 23 | | | | | | | | | | | | | |
| | | | | | | | | | | | | | | (intestinal), CRIP1 | | | | | | | | | | | | | | |
| 33352_at | H2BFQ | X57985 | 6 | Pass | 29.67 | 10 27 | 13 | Pass | TRUE | FALSE | FALSE | 13 23 | 2.24 | H2B histone family, | 1q21-q23 | | | | | | | | | | | | | |
| | | | | | | | | | | | | | | member Q: H2BFQ | | | | | | | | | | | | | | |
| 38724_at | KIAA0515 | AB011087 | 6 | Pass | 10.17 | 1 83 | 13 | Pass | TRUE | FALSE | FALSE | 4 54 | 2.24 | KIAA0515 protein, | | | | | | | | | | | | | | |
| | | | | | | | | | | | | | | KIAA0515 | | | | | | | | | | | | | | |
| 1505_at | TYMS | D00596 | 5 | Pass | 7.00 | 2 00 | 8 | Pass | TRUE | FALSE | FALSE | 3 13 | 2.24 thymidylate synthetase, | 18p11 32 | | | | | | | | | | | | | | |
| | | | | | | | | | | | | | | IYMS | | | | | | | | | | | | | | |
| 36984_f_at | HPR | X89214 | 5 | Pass | 7.00 | 2 83 | 8 | Pass | TRUE | FALSE | FALSE | 3 13 | 2.24 | haptoglobin, haptoglobin- | 16q22 1 | | | | | | | | | | | | | |
| | | | | | | | | | | | | | | related protein HP, HPR | | | | | | | | | | | | | | |
| 35302_at | TAP | AJ132312 | 6 | Pass | 53.50 | 14.82 | 13 | Pass | TRUE | FALSE | FALSE | 23.92 | 2.24 | nuclear RNA export factor | | | | | | | | | | | | | | |
| | | | | | | | | | | | | | | 1 (Mex67, yeast, | | | | | | | | | | | | | | |
| | | | | | | | | | | | | | | homolog, NXF1 | | | | | | | | | | | | | | |
| 36996_at | OS-9 | U41635 | 6 | Pass | 66 00 | 19 85 | 13 | Pass | TRUE | FALSE | FALSE | 29.54 | 2.23 | amplified in osteosarcoma, | 12q13 | | | | | | | | | | | | | |
| | | | | | | | | | | | | | | OS-9 | | | | | | | | | | | | | | |
| 38672_at | PPRIR10 | Y13247 | 6 | Pass | 22 83 | 7.78 | 13 | Pass | TRUE | FALSE | FALSE | 10 23 | 2.23 | protein phosphatase 1, | 6p21.3 | | | | | | | | | | | | | |
| | | | | | | | | | | | | | | regulatory subunit 10, | | | | | | | | | | | | | | |
| | | | | | | | | | | | | | | PPPIR10 | | | | | | | | | | | | | | |
| 41189_at | TNFRSF12 | Y09302 | 6 | Pass | 11 50 | 4 18 | 13 | Pass | TRUE | FALSE | FALSE | 5.15 | 2.23 | tumor necrosis factor | 1p36 2 | | | | | | | | | | | | | |
| | | | | | | | | | | | | | | receptor superfamily, | | | | | | | | | | | | | | |
| | | | | | | | | | | | | | | member 12 (translocating | | | | | | | | | | | | | | |
| | | | | | | | | | | | | | | chain-association | | | | | | | | | | | | | | |
| | | | | | | | | | | | | | | membrane protein): | | | | | | | | | | | | | | |
| 38432_at | ISG15 | AA203213 | 6 | Pass | 16 50 | 13.82 | 10 Pass | TRUE | FALSE | FALSE | 7.40 | 2.23 | interferon-stimulated | 1 | | | | | | | | | | | | | | |
| | | | | | | | | | | | | | | protein, 15 kDa, ISG15 | | | | | | | | | | | | | | |
| 38279_at | GNAZ | D90150 | 6 | Pass | 11 83 | 4.62 | 13 | Pass | TRUE | FALSE | FALSE | 5.31 | 2.23 | guanine nucleotide binding | 22q11 22 | | | | | | | | | | | | | |
| | | | | | | | | | | | | | | protein (G protein, alpha | | | | | | | | | | | | | | |
| | | | | | | | | | | | | | | z, polypeptide, GNAZ | | | | | | | | | | | | | | |
| 38585_at | HBG2 | M91036 | 6 | Pass | 101.50 | 94.05 | 12 | Pass | TRUE | FALSE | FALSE | 45.58 | 2.23 | hemoglobin, gamma | 11p15 5 | | | | | | | | | | | | | |
| | | | | | | | | | | | | | | A, hemoglobin, gamma G, | | | | | | | | | | | | | | |
| | | | | | | | | | | | | | | HBG1, HBG2 | | | | | | | | | | | | | | |
| 38112_g_at | CSPG2 | X15998 | 6 | Pass | 69.67 | 52 00 | 13 | Pass | TRUE | FALSE | FALSE | 31 31 | 2.23 | chrondrotin sulfate | 5q14 3 | | | | | | | | | | | | | |
| | | | | | | | | | | | | | | proteoglycn 2 (vcrsican), | | | | | | | | | | | | | | |
| | | | | | | | | | | | | | | CSPG2 | | | | | | | | | | | | | | |
| 1274_s_at | CDC34 | L22005 | 6 | Pass | 10.17 | 3 25 | 7 | Pass | TRUE | FALSE | FALSE | 4 57 | 2.22 | cell division cycle 34, | 19p13 3 | | | | | | | | | | | | | |
| | | | | | | | | | | | | | | CDC34 | | | | | | | | | | | | | | |
| 1643_g_at | MTA1 | U35113 | 6 | Pass | 8.00 | 2 19 | 10 | Pass | TRUE | FALSE | FALSE | 3 60 | 2.22 | metastasis associated 1. | | | | | | | | | | | | | | |
| | | | | | | | | | | | | | | MTA1 | | | | | | | | | | | | | | |
| 243_g_at | MAP4 | M64571 | 6 | Pass | 10.00 | 3 63 | 10 | Pass | TRUE | FALSE | FALSE | 4 50 | 2.22 | microtubule-associated | 3p21 | | | | | | | | | | | | | |
| | | | | | | | | | | | | | | protein 4, MAP4 | | | | | | | | | | | | | | |
| 37346_at | ARF5 | M57567 | 6 | Pass | 46.67 | 18 51 | 12 | Pass | TRUE | FALSE | FALSE | 21.00 | 2.22 | ADP-ribosylanon factor 5, | 7q31 3 | | | | | | | | | | | | | |
| | | | | | | | | | | | | | | ARF5 | | | | | | | | | | | | | | |
| 37959_at | KIAA0154 | D63876 | 6 | Pass | 20 00 | 73 8 | 13 | Pass | TRUE | FALSE | FALSE | 9 00 | 2.22 | KIAA0154 protein, ADP- | 17 | | | | | | | | | | | | | |
| | | | | | | | | | | | | | | ribosylation factor beidiog | | | | | | | | | | | | | | |
| | | | | | | | | | | | | | | protein GGA3, KIAA0154 | | | | | | | | | | | | | | |
| 38663_at | BCRP1 | AI033692 | 6 | Pass | 23 50 | 7 23 | 12 | Pass | TRUE | FALSE | FALSE | 10 58 | 2.22 | Breakpoint cluster region | 14q24 1 | | | | | | | | | | | | | |
| | | | | | | | | | | | | | | protein, uterine | | | | | | | | | | | | | | |
| | | | | | | | | | | | | | | leromyoma, 1, barrier to | | | | | | | | | | | | | | |
| | | | | | | | | | | | | | | autointegration factor. | | | | | | | | | | | | | | |
| 37844 at | UNK_AI20 | Ai263885 | 6 | Pass | 20.67 | 8.87 | 13 | Pass | TRUE | FALSE | FALSE | 9.31 | 2.32 | class 1 cytokine receptor; | 19p13.11 | | | | | | | | | | | | | |
| | | | | | | | | | | | | | | WXS-1 | | | | | | | | | | | | | | |
| 32607_at | BASP1 | AF039656 | 6 | Pass | 36 33 | 21.37 | 13 | Pass | TRUE | FALSE | FALSE | 16.38 | 2.22 | brain acid-soluble protein | 5p14-15 | | | | | | | | | | | | | |
| | | | | | | | | | | | | | | 1, BASP1 | | | | | | | | | | | | | | |
| 33396_at | GSTP1 | U12472 | 6 | Pass | 70 17 | 32.53 | 13 | Pass | TRUE | FALSE | FALSE | 31.69 | 2.21 | glutathione S-transferase | 11q13 | | | | | | | | | | | | | |
| | | | | | | | | | | | | | | p1, GSTP1 | | | | | | | | | | | | | | |
| 39330 s at | ACTN1 | M95178 | 6 | Pass | 15 83 | 5 12 | 13 | Pass | TRUE | FALSE | FALSE | 7 15 | 2.21 | actinin, alpha 1: ACTN1 | 14q24 | | | | | | | | | | | | | |
| 38703_at | UNK_AF0 | AF005050 | 6 | Pass | 16 17 | 4 07 | 13 | Pass | TRUE | FALSE | FALSE | 7.31 | 2.21 | aspartyl aminopeptide; | | | | | | | | | | | | | | |
| | | | | | | | | | | | | | | DNPEP | | | | | | | | | | | | | | |
| 40723_at | SIT | AJ010059 | 6 | Pass | 16 17 | 4 62 | 13 | Pass | TRUE | FALSE | FALSE | 7 31 | 2.21 | SHP2 interacting | | | | | | | | | | | | | | |
| | | | | | | | | | | | | | | transmembrane adaptor, | | | | | | | | | | | | | | |
| | | | | | | | | | | | | | | SIT | | | | | | | | | | | | | | |
| 464_s_at | IF135 | U72882 | 6 | Pass | 16 17 | 8 98 | 13 | Pass | TRUE | FALSE | FALSE | 7 31 | 2.21 | interferon-induced protein | 17q21 | | | | | | | | | | | | | |
| | | | | | | | | | | | | | | 35 IF135 | | | | | | | | | | | | | | |
| 32226_at | MAP4 | M64571 | 6 | Pass | 12 67 | 4 72 | 11 | Pass | TRUE | FALSE | FALSE | 5 73 | 2.21 | microtubule-associated | 3p21 | | | | | | | | | | | | | |
| | | | | | | | | | | | | | | protein 4, MAP4 | | | | | | | | | | | | | | |
| 868_at | TAF2H | U13991 | 6 | Pass | 33 33 | 7 76 | 13 | Pass | TRUE | FALSE | FALSE | 15 08 | 2.21 | TATA box binding protein | 11p15 3 | | | | | | | | | | | | | |
| | | | | | | | | | | | | | | (TBP)-associated factor, | | | | | | | | | | | | | | |
| | | | | | | | | | | | | | | RNA polymerase II, II, | | | | | | | | | | | | | | |
| | | | | | | | | | | | | | | 30kD, TAF2H | | | | | | | | | | | | | | |
| 38056_at | KIAA0195 | D83779 | 6 | Pass | 17 33 | 4 55 | 13 | Pass | TRUE | FALSE | FALSE | 7 85 | 2.21 | KIAA0195 gene product, | 17 | | | | | | | | | | | | | |
| | | | | | | | | | | | | | | KIAA0195 | | | | | | | | | | | | | | |
| 37898_r_at | TFF3 | AI985964 | 4 | Pass | 37 00 | 14 72 | 8 | Pass | TRUE | FALSE | FALSE | 16 75 | 2.21 | trefoil factor 3 (intestinal); | 21q22 3 | | | | | | | | | | | | | |
| | | | | | | | | | | | | | | TFF3 | | | | | | | | | | | | | | |
| 654_at | MX11 | L07648 | 6 | Pass | 14 17 | 7 36 | 12 | Pass | TRUE | FALSE | FALSE | 6 42 | 2.21 | MAX-interacting protein | 10q24- | | | | | | | | | | | | | |
| | | | | | | | | | | | | | | 1, MX11 | q25 | | | | | | | | | | | | | |
| 33602_at | EDG6 | AJ000479 | 6 | Pass | 27 17 | 6 34 | 13 | Pass | TRUE | FALSE | FALSE | 12 31 | 2.21 | endothelial differentiation, | 19p13 3 | | | | | | | | | | | | | |
| | | | | | | | | | | | | | | G-protein-coupled receptor | | | | | | | | | | | | | | |
| | | | | | | | | | | | | | | 6, EDG6 | | | | | | | | | | | | | | |
| 39308_r_at | UNK_X81 | X81637 | 6 | Pass | 9 67 | 2 88 | 13 | Pass | TRUE | FALSE | FALSE | 4.38 | 2.20 | clathrin, light polypeptide | 4q2-q3 | | | | | | | | | | | | | |
| | | | | | | | | | | | | | | (Lcb), CLTB | | | | | | | | | | | | | | |
| 37904 s at | GNL1 | X66436 | 6 | Pass | 6 17 | 1 33 | 10 | Pass | TRUE | FALSE | FALSE | 2.80 | 2.20 | | | | | | | | | | | | | | | |
| 1395_at | ARHC | L25081 | 5 | Pass | 13 00 | 7 11 | 11 | Pass | TRUE | FALSE | FALSE | 5.91 | 2.20 | ras homolog gene family, | 1p21-p13 | | | | | | | | | | | | | |
| | | | | | | | | | | | | | | member C, ARHC | | | | | | | | | | | | | | |
| 39122_at | GPI | K03515 | 6 | Pass | 33 00 | 7 87 | 13 | Pass | TRUE | FALSE | FALSE | 15 00 | 2.20 | glucose phosphate | 19q13 1 | | | | | | | | | | | | | |
| | | | | | | | | | | | | | | isomerase; GPI | | | | | | | | | | | | | | |
| 609_f_{—l at} | MT1B | M13485 | 4 | Pass | 27 00 | 5 72 | 11 | Pass | TRUE | FALSE | FALSE | 12 27 | 2.20 | netallothronein 1B | 16q13 | | | | | | | | | | | | | |
| | | | | | | | | | | | | | | (functional), MT1B | | | | | | | | | | | | | | |
| 34358_at | RPL23L | Z49254 | 6 | Pass | 22 67 | 6 38 | 13 | Pass | TRUE | FALSE | FALSE | 10 31 | 2.20 | ribosomal protein | L23, | 11p15 5 | | | | | | | | | | | | |
| | | | | | | | | | | | | | | like, RPL23L | | | | | | | | | | | | | | |
| 35485_at | GRM4 | X80818 | 5 | Pass | 53.40 | 13 24 | 7 | Pass | TRUE | FALSE | FALSE | 24 29 | 2.20 | glutamate receptor, 6p21.3 | | | | | | | | | | | | | | |
| | | | | | | | | | | | | | | metaborphic 4, GRM4 | | | | | | | | | | | | | | |
| 32574_at | SMPD1 | X59060 | 6 | Pass | 13.17 | 4 17 | 13 | Pass | TRUE | FALSE | FALSE | 6 00 | 2.19 | sphingonyclin | 11p15 4- | | | | | | | | | | | | | |
| | | | | | | | | | | | | | | phosphodrestatase 1, acid | p15 1 | | | | | | | | | | | | | |
| | | | | | | | | | | | | | | lysosomial (acid | | | | | | | | | | | | | | |
| | | | | | | | | | | | | | | sphingonyelinase), | | | | | | | | | | | | | | |
| | | | | | | | | | | | | | | SMPD1 | | | | | | | | | | | | | | |
| 38259_at | STXBP2 | AB002559 | 6 | Pass | 12 50 | 4 32 | 10 | Pass | TRUE | FALSE | FALSE | 5.70 | 2.19 | syntaxin binding protein 2: | 10p13 3- | | | | | | | | | | | | | |
| | | | | | | | | | | | | | | STXBP2 | | | | | | | | | | | | | | |
| 36417_s_at | UNK_AF0 | AF035295 | 6 | Pass | 15.17 | 5 23 | 12 | Pass | TRUE | FALSE | FALSE | 6 92 | 2.19 | acctyl-Coenzyme A | 3p23-p22 | | | | | | | | | | | | | |
| | | | | | | | | | | | | | | acyltransferase 1 | | | | | | | | | | | | | | |
| | | | | | | | | | | | | | | (petoxisomal 3-oxoacyl- | | | | | | | | | | | | | | |
| | | | | | | | | | | | | | | Coenzyme A thiolase), | | | | | | | | | | | | | | |
| | | | | | | | | | | | | | | ACAA1 | | | | | | | | | | | | | | |
| 35154_at | UNK_W68 | W68046 | 6 | Pass | 52.33 | 10 75 | 8 | Pass | TRUE | FALSE | FALSE | 23.88 | 2.19 | Hypothetical protein | | | | | | | | | | | | | | |
| | | | | | | | | | | | | | | FLJ20386, FLJ20386 | | | | | | | | | | | | | | |
| 38087_s_at | S100A4 | W72186 | 6 | Pass | 177 67 | 62.89 | 13 | Pass | TRUE | FALSE | FALSE | 81.08 | 2.19 | S100 calcium-binding | 1q21 | | | | | | | | | | | | | |
| | | | | | | | | | | | | | | protein A4 (calcium | | | | | | | | | | | | | | |
| | | | | | | | | | | | | | | protein, calvasculin, | | | | | | | | | | | | | | |
| | | | | | | | | | | | | | | mectastasm, murine | | | | | | | | | | | | | | |
| | | | | | | | | | | | | | | placental homolog), | | | | | | | | | | | | | | |
| | | | | | | | | | | | | | | S100A4 | | | | | | | | | | | | | | |
| 1583_at | TNFRSF1B | M32315 | 6 | Pass | 92.83 | 44.60 | 13 | Pass | TRUE | FALSE | FALSE | 42.38 | 2.19 | tumor necrosis factor | 1p36.3- | | | | | | | | | | | | | |
| | | | | | | | | | | | | | | receptor superfamily, | p36.2 | | | | | | | | | | | | | |
| | | | | | | | | | | | | | | member 1B; TNFRSF1B | | | | | | | | | | | | | | |
| 214_at | MXS1 | M97676 | 5 | Pass | 10 40 | 4.51 | 8 | Pass | TRUE | FALSE | FALSE | 4 75 | 2.19 | msh (Drosphilia) homco | 4p16 3- | | | | | | | | | | | | | |
| | | | | | | | | | | | | | | box homolog 1 (formerly | p16 1 | | | | | | | | | | | | | |
| | | | | | | | | | | | | | | homeo box 7), MSX1 | | | | | | | | | | | | | | |
| 38789_at | TKT | L12711 | 6 | Pass | 97.00 | 62 34 | 13 | Pass | TRUE | FALSE | FALSE | 44 31 | 2.19 | transketolase (Wenucke- | 3p14 3 | | | | | | | | | | | | | |
| | | | | | | | | | | | | | | Kusakoff syndrome), | | | | | | | | | | | | | | |
| 40106_at | EIB-AP5 | AJ007509 | 6 | Pass | 32.83 | 11 87 | 13 | Pass | TRUE | FALSE | FALSE | 15.00 | 2.19 | EIB-55kDa-associated | | | | | | | | | | | | | | |
| | | | | | | | | | | | | | | protein 5, EIB-AP5 | | | | | | | | | | | | | | |
| 38831_f_at | UNK_AF0 | AF053356 | 6 | Pass | 67.17 | 23 27 | 13 | Pass | TRUE | FALSE | FALSE | 30 69 | 2.19 | guanine nucleotide binding | 7q21 3- | | | | | | | | | | | | | |
| | | | | | | | | | | | | | | protein (G protein), beta | q22 1 | | | | | | | | | | | | | |
| | | | | | | | | | | | | | | polypeptide 2, GNB2 | | | | | | | | | | | | | | |
| 1804_at | KLK3 | X07730 | 6 | Pass | 6 33 | 1 51 | 10 | Pass | TRUE | FALSE | FALSE | 2 90 | 2.18 | kallikrein 3, (prostate | 19q13 | | | | | | | | | | | | | |
| | | | | | | | | | | | | | | specific antigen), KLK3 | | | | | | | | | | | | | | |
| 38735_at | KIAA0513 | AB011085 | 6 | Pass | 24.33 | 8 55 | 13 | Pass | TRUE | FALSE | FALSE | 11 15 | 2.18 | KIAA0513 gene product, | | | | | | | | | | | | | | |
| | | | | | | | | | | | | | | KIAA0513 | | | | | | | | | | | | | | |
| 41443 at | TIC | U63127 | 6 | Pass | 25 67 | 6 02 | 13 | Pass | TRUE | FALSE | FALSE | 11 77 | 2.18 | SEC7 homolog, TIC | 2q13 | | | | | | | | | | | | | |
| 38119_at | GYPC | X12496 | 6 | Pass | 56 33 | 19 66 | 13 | Pass | TRUE | FALSE | FALSE | 25 85 | 2.18 | glcophorin C (Gerbich | 2q14-q21 | | | | | | | | | | | | | |
| | | | | | | | | | | | | | | blood group), GYPC | | | | | | | | | | | | | | |
| 39594 f at | MT1H | R93527 | 6 | Pass | 28 67 | 10 27 | 13 | Pass | TRUE | FALSE | FALSE | 13 15 | 2.18 | metallothensein 1H, | 16q13 | | | | | | | | | | | | | |
| 41421_at | KIAA0909 | AB020716 | 6 | Pass | 12 17 | 5.12 | 12 | Pass | TRUE | FALSE | FALSE | 5.58 | 2.18 | KIAA0909 protein, | | | | | | | | | | | | | | |
| | | | | | | | | | | | | | | KIAA0909 | | | | | | | | | | | | | | |
| 40712_at | ADAM8 | D26579 | 6 | Pass | 44 00 | 15 09 | 13 | Pass | TRUE | FALSE | FALSE | 20 23 | 2.17 | a disintegrin and | 10q26 3 | | | | | | | | | | | | | |
| | | | | | | | | | | | | | | metalloprotease domain 8, | | | | | | | | | | | | | | |
| | | | | | | | | | | | | | | ADAM8 | | | | | | | | | | | | | | |
| 40569_at | ZNF42 | M58297 | 6 | Pass | 8 33 | 1.51 | 12 | Pass | TRUE | FALSE | FALSE | 3.83 | 2.17 | zinc finger protein 42 | 19q13 2- | | | | | | | | | | | | | |
| | | | | | | | | | | | | | | (myeloid-specific retinoic | q13 4 | | | | | | | | | | | | | |
| | | | | | | | | | | | | | | acid- responsive), ZNF-42 | | | | | | | | | | | | | | |
| 283_at | UQCRC1 | L16842 | 6 | Pass | 27 17 | 10 46 | 12 | Pass | TRUE | FALSE | FALSE | 12 50 | 2.17 | ribiqiunol-cytochrome c | 3p21 3 | | | | | | | | | | | | | |
| | | | | | | | | | | | | | | reductase core protein 1, | | | | | | | | | | | | | | |
| | | | | | | | | | | | | | | UQCRC 1 | | | | | | | | | | | | | | |
| 40100_at | LFP40 | U72206 | 6 | Pass | 16 00 | 5 66 | 11 | Pass | TRUE | FALSE | FALSE | 7.36 | 2.17 rho/rac guanine nucleotide | 1 | | | | | | | | | | | | | | |
| | | | | | | | | | | | | | | exchange factor (GEF) 2, | | | | | | | | | | | | | | |
| | | | | | | | | | | | | | | ARBGEF2 | | | | | | | | | | | | | | |
| 1578_g_at | ERCC1 | M13194 | 6 | Pass | 14 33 | 4 50 | 10 | Pass | TRUE | FALSE | FALSE | 6.60 | 2.17 | excision repair cross- | 19q13 2- | | | | | | | | | | | | | |
| | | | | | | | | | | | | | | complementing rodent | q13 3 | | | | | | | | | | | | | |
| | | | | | | | | | | | | | | repair deficiency, | | | | | | | | | | | | | | |
| | | | | | | | | | | | | | | complementation group 1 | | | | | | | | | | | | | | |
| | | | | | | | | | | | | | | (includes overlapping | | | | | | | | | | | | | | |
| | | | | | | | | | | | | | | antisense sequence), | | | | | | | | | | | | | | |
| | | | | | | | | | | | | | | ERCC1 | | | | | | | | | | | | | | |
| 37329_at | NDUFV1 | AF053070 | 6 | Pass | 17.00 | 6 07 | 12 | Pass | TRUE | FALSE | FALSE | 7.83 | 2.17 | NADH dehydrogenase | 11q13 | | | | | | | | | | | | | |
| | | | | | | | | | | | | | | (ubiquinone) flavoprotein | | | | | | | | | | | | | | |
| | | | | | | | | | | | | | | 1 (51kD), NDUFV1 | | | | | | | | | | | | | | |
| 41177 at | UNK AW | AW024285 | 6 | Pass | 35.33 | 17 08 | 13 | Pass | TRUE | FALSE | FALSE | 16.31 | 2.17 | | | | | | | | | | | | | | | |
| 41332_at | POLR2E | D38251 | 6 | Pass | 19.50 | 7 34 | 7 | Pass | TRUE | FALSE | FALSE | 9.00 | 2.17 | palymerase (RNA) II | 19p13 3 | | | | | | | | | | | | | |
| | | | | | | | | | | | | | | (DNA directed) | | | | | | | | | | | | | | |
| | | | | | | | | | | | | | | polypeptide E (25kD). | | | | | | | | | | | | | | |
| | | | | | | | | | | | | | | POLR2E | | | | | | | | | | | | | | |
| 1061 _at | IL10RA | U00672 | 6 | Pass | 31 00 | 7 77 | 13 | Pass | TRUE | FALSE | FALSE | 14.31 | 2.17 | interleukin 10 receptor, | 11q23 | | | | | | | | | | | | | |
| | | | | | | | | | | | | | | alpha, IL10RA | | | | | | | | | | | | | | |
| 32529_at | P63 | X69910 | 6 | Pass | 14 00 | 8 88 | 13 | Pass | TRUE | FALSE | FALSE | 6 46 | 2.17 | transmembrane protein | 12 | | | | | | | | | | | | | |
| | | | | | | | | | | | | | | (63kD), endoplasmic | | | | | | | | | | | | | | |
| | | | | | | | | | | | | | | reticulum/Golgi | | | | | | | | | | | | | | |
| | | | | | | | | | | | | | | intermediate compartment, | | | | | | | | | | | | | | |
| | | | | | | | | | | | | | | P63 | | | | | | | | | | | | | | |
| 32681_at | SLC9A1 | S68616 | 6 | Pass | 24 67 | 6 77 13 | Pass | TRUE | FALSE | FALSE | 11.38 | 2.17 | solute carier family 9 | 1p36 1- | | | | | | | | | | | | | | |
| | | | | | | | | | | | | | | (sodium/hydrogen | p35 | | | | | | | | | | | | | |
| | | | | | | | | | | | | | | exchanger), isoform 1 | | | | | | | | | | | | | | |
| | | | | | | | | | | | | | | (antiporter, Na+/II+, | | | | | | | | | | | | | | |
| | | | | | | | | | | | | | | amioide sensitive). | | | | | | | | | | | | | | |
| | | | | | | | | | | | | | | SLC9A1 | | | | | | | | | | | | | | |
| 38354_at | CEBPB | X52560 | 6 | Pass | 74.00 | 37.56 | 13 | Pass | TRUE | FALSE | FALSE | 34.15 | 2.17 | CCAAT/enhancer binding | 20q13.1 | | | | | | | | | | | | | |
| | | | | | | | | | | | | | | protein (C/EBP), beta; | | | | | | | | | | | | | | |
| | | | | | | | | | | | | | | CEBPB | | | | | | | | | | | | | | |
| 38397 at | UNK U09 | U09196 | 6 | Pass | 19.33 | 5 24 | 13 | Pass | TRUE | FALSE | FALSE | 8.92 | 2.17 | | | | | | | | | | | | | | | |
| 39339_at | KIAA0792 | AB018335 | 6 | Pass | 20.83 | 5 19 | 13 | Pass | TRUE | FALSE | FALSE | 9.62 | 2.17 | KIAA0792 gene product, | | | | | | | | | | | | | | |
| | | | | | | | | | | | | | | KIAA0792 | | | | | | | | | | | | | | |
| 41084 at | UNK A165 | A1659108 | 6 | Pass | 12.50 | 3 62 | 13 | Pass | TRUE | FALSE | FALSE | 5.77 | 2.17 | | | | | | | | | | | | | | | |
| 35826_at | SUPT5H | AF040253 | 6 | Pass | 16 83 | 5 08 | 9 | Pass | TRUE | FALSE | FALSE | 7.78 | 2.16 | suppressor of Ty | 19q13 | | | | | | | | | | | | | |
| | | | | | | | | | | | | | | (S. cerevisiae) 5 homolog, | | | | | | | | | | | | | | |
| | | | | | | | | | | | | | | SUPT5H | | | | | | | | | | | | | | |
| 34231_at | UNK_AF0 | AF074606 | 6 | Pass | 17.50 | 6 86 | 11 | Pass | TRUE | FALSE | FALSE | 8 09 | 2.16 | histone acetyltransferase, | Xq21 | | | | | | | | | | | | | |
| | | | | | | | | | | | | | | HBOA | | | | | | | | | | | | | | |
| 33261 at | HLA-DRB | M16941 | 6 | Pass | 146 83 | 54 47 | 13 | Pass | TRUE | FALSE | FALSE | 67.92 | 2.16 | major hostocompatibility | 6p21 3 | | | | | | | | | | | | | |
| | | | | | | | | | | | | | | complex class II, DR beta | | | | | | | | | | | | | | |
| | | | | | | | | | | | | | | 1, IILA-DRB1 | | | | | | | | | | | | | | |
| 33213_g_at | RRP1 | AF006751 | 6 | Pass | 8 83 | 5.12 | 11 | Pass | TRUE | FALSE | FALSE | 4 09 | 2.16 | ribosome binding protein 1 | 20p12 | | | | | | | | | | | | | |
| | | | | | | | | | | | | | | (dog 180kD homolog); | | | | | | | | | | | | | | |
| | | | | | | | | | | | | | | RRBP1 | | | | | | | | | | | | | | |
| 37148_at | LILRB3 | AR025533 | 6 | Pass | 44 50 | 25 98 | 13 | Pass | TRUE | FALSE | FALSE | 20 62 | 2.16 | leukocytic immunoglobulin | 19q13 4 | | | | | | | | | | | | | |
| | | | | | | | | | | | | | | like receptor subfamily B | | | | | | | | | | | | | | |
| | | | | | | | | | | | | | | (with TM and ITIM | | | | | | | | | | | | | | |
| | | | | | | | | | | | | | | domains), member 3, | | | | | | | | | | | | | | |
| | | | | | | | | | | | | | | LILRB3 | | | | | | | | | | | | | | |
| 35336 at | K1AA0668 | AL021707 | 6 | Pass | 105 00 | 25 46 | 13 | Pass | TRUE | FALSE | FALSE | 48 77 | 2.15 | | | | | | | | | | | | | | | |
| 505_at | CDC37 | U43077 | 6 | Pass | 48.50 | 9 75 | 13 | Pass | TRUE | FALSE | FALSE | 2 254 | 2.15 | CDC37 (cell division cyclic | 19 | | | | | | | | | | | | | |
| | | | | | | | | | | | | | | 37, S cerevisiae, | | | | | | | | | | | | | | |
| | | | | | | | | | | | | | | homolog), CDC37 | | | | | | | | | | | | | | |
| 37706_at | GLG1 | U25811 | 6 | Pass | 14.67 | 2 50 | 11 | Pass | TRUE | FALSE | FALSE | 6 82 | 2.15 | Golgi apparatus protein 1, | 16q22- | | | | | | | | | | | | | |
| | | | | | | | | | | | | | | GLG1 | q23 | | | | | | | | | | | | | |
| 37650_at | UNK_U41 | U41315 | 6 | Pass | 43 33 | 11 57 | 13 | Pass | TRUE | FALSE | FALSE | 20.15 | 2.15 | unknown, ring finger | 7q34 | | | | | | | | | | | | | |
| | | | | | | | | | | | | | | protein 1, MKRN1 | | | | | | | | | | | | | | |
| 333660_at | KIAA1004 | AB023221 | 6 | Pass | 20 33 | 3 01 | 13 | Pass | TRUE | FALSE | FALSE | 9.46 | 2.15 | 1-box and leucine-rich | | | | | | | | | | | | | | |
| | | | | | | | | | | | | | | repeat protein 11, FBXL11 | | | | | | | | | | | | | | |
| 36933_at | NDRG1 | D87953 | 6 | Pass | 19 67 | 6 25 | 13 | Pass | TRUE | FALSE | FALSE | 9 15 | 2.15 | N-myc downstream | 8 | | | | | | | | | | | | | |
| | | | | | | | | | | | | | | regulated, NDRG1 | | | | | | | | | | | | | | |
| 33824_at | KRT8 | X74929 | 6 | Pass | 24.17 | 8.61 | 12 | Pass | TRUE | FALSE | FALSE | 11 25 | 2.15 | CGI-39 protein, keratin 8, | 12q13 | | | | | | | | | | | | | |
| | | | | | | | | | | | | | | KRT8, LOC51079 | | | | | | | | | | | | | | |
| 36634_at | BTG2 | U73649 | 6 | Pass | 38 67 | 16 39 | 13 | Pass | TRUE | FALSE | FALSE | 18 00 | 2.15 | BTG family, member 2 | 1q32 | | | | | | | | | | | | | |
| | | | | | | | | | | | | | | BTG2 | | | | | | | | | | | | | | |
| 38641 at | UNK AJ1 | AJ133115 | 6 | Pass | 15.83 | 4 75 | 8 | Pass | TRUE | FALSE | FALSE | 7 38 | 2.15 | | | | | | | | | | | | | | | |
| 40695_at | IMPDH1 | J05272 | 6 | Pass | 35.67 | 14.80 | 13 | Pass | TRUE | FALSE | FALSE | 16.62 | 2.15 | IMP (inosine | 7q31 3- | | | | | | | | | | | | | |
| | | | | | | | | | | | | | | monophosphate) | q32 | | | | | | | | | | | | | |
| | | | | | | | | | | | | | | dehydrogenase 1, | | | | | | | | | | | | | | |
| 39347_at | CLAPS2 | X97074 | 6 | Pass | 50 67 | 17 93 | 13 | Pass | TRUE | FALSE | FALSE | 23.62 | 2.15 | adaptor-related protein | 19q13 2- | | | | | | | | | | | | | |
| | | | | | | | | | | | | | | complex 2, sigma 1 | q13 3 | | | | | | | | | | | | | |
| | | | | | | | | | | | | | | subunit, AP2S1 | | | | | | | | | | | | | | |
| 39134_at | TOM1 | AJ006973 | 6 | Pass | 7.50 | 2 66 | 10 | Pass | TRUE | FALSE | FALSE | 3.50 | 2.14 | target of myb1 (chicken) | 22q13 1 | | | | | | | | | | | | | |
| | | | | | | | | | | | | | | homolog, TOM1 | | | | | | | | | | | | | | |
| 35770_at | ATP6S1 | D16469 | 6 | Pass | 44 00 | 20 62 | 13 | Pass | TRUE | FALSE | FALSE | 20 54 | 2.14 | ATPase, H+ transporting, | Xq28 | | | | | | | | | | | | | |
| | | | | | | | | | | | | | | lysosomal (vacuolar | | | | | | | | | | | | | | |
| | | | | | | | | | | | | | | proton pump), subunit 1 | | | | | | | | | | | | | | |
| 36130_{—l f}_at | MTIE | R92331 | 6 | Pass | 25.67 | 6 95 | 12 | Pass | TRUE | FALSE | FALSE | 12.00 | 2.14 | metallothonein 1E | 16q13 | | | | | | | | | | | | | |
| | | | | | | | | | | | | | | (functional), MTIE | | | | | | | | | | | | | | |
| 506_s_at | STAT5A | U43185 | 6 | Pass | 23.83 | 4 88 | 13 | Pass | TRUE | FALSE | FALSE | 11.15 | 2.14 | signal transducer and | 17q11 2 | | | | | | | | | | | | | |
| | | | | | | | | | | | | | | activator of transcription | | | | | | | | | | | | | | |
| | | | | | | | | | | | | | | SA, STAT5A | | | | | | | | | | | | | | |
| 32192_g_at | ZNF144 | D13969 | 6 | Pass | 18.00 | 4.20 | 7 | Pass | TRUE | FALSE | FALSE | 8 43 | 2.14 | sinc finger protein 144 | 17 | | | | | | | | | | | | | |
| | | | | | | | | | | | | | | (Mel-18), ZNF144 | | | | | | | | | | | | | | |
| 32193_at | SLC25A11 | AF070548 | 6 | Pass | 11 17 | 4 31 | 13 | Pass | TRUE | FALSE | FALSE | 5 23 | 2.13 | solute carrier family 25 | 17p13 3 | | | | | | | | | | | | | |
| | | | | | | | | | | | | | | (mitochondrial carrier, | | | | | | | | | | | | | | |
| | | | | | | | | | | | | | | oxogluterate carrier), | | | | | | | | | | | | | | |
| | | | | | | | | | | | | | | member II: SLC25A11 | | | | | | | | | | | | | | |
| 40980 at | UNK W26 | W26477 | 6 | Pass | 11 17 | 2 93 | 13 | Pass | TRUE | FALSE | FALSE | 5 23 | 2.13 | | | | | | | | | | | | | | | |
| 32717_at | NEURL | AF029729 | 4 | Pass | 27.75 | 5.74 | 8 | Pass | TRUE | FALSE | FALSE | 13.00 | 2.13 | neuralized (Drosophila)- | 10q25.1 | | | | | | | | | | | | | |
| | | | | | | | | | | | | | | like, NEURL | | | | | | | | | | | | | | |
| 38700_at | CSRP1 | M33146 | 6 | Pass | 11.83 | 3 66 | 11 | Pass | TRUE | FALSE | FALSE | 5 55 | 2.13 | cysteine and glycine-rich | 1q32 | | | | | | | | | | | | | |
| | | | | | | | | | | | | | | protein 1, CSRP1 | | | | | | | | | | | | | | |
| 38718_at | DKFZP586 | AL050101 | 6 | Pass | 8 00 | 2 10 | 12 | Pass | TRUE | FALSE | FALSE | 3.75 | 2.13 | DKFZP586F1519 protein, | | | | | | | | | | | | | | |
| | | | | | | | | | | | | | | DKFZP586E1519 | | | | | | | | | | | | | | |
| 33390 at | UNK AAZ | AA203487 | 6 | Pass | 83 67 | 49 40 | 9 | Pass | TRUE | FALSE | FALSE | 24 30 | 2.13 | glutothione S transferase | 11q13 | | | | | | | | | | | | | |
| | | | | | | | | | | | | | | p; GSTP1 | | | | | | | | | | | | | | |
| 39778_at | MGAT1 | M55621 | 6 | Pass | 39 67 | 12 47 | 13 | Pass | TRUE | FALSE | FALSE | 18 62 | 2.13 | mannosyl (alpha-1,3-)- | 5q35 | | | | | | | | | | | | | |
| | | | | | | | | | | | | | | glcoprotein beta-1,2-N- | | | | | | | | | | | | | | |
| | | | | | | | | | | | | | | acetylglucosamminyltransfer | | | | | | | | | | | | | | |
| | | | | | | | | | | | | | | ase, MGAT1 | | | | | | | | | | | | | | |
| 40153_at | ABCB2 | X57522 | 6 | Pass | 29 50 | 8 67 | 13 | Pass | TRUE | FALSE | FALSE | 13 85 | 2.13 | ATP-binding cassette, sub- | 6p21 3 | | | | | | | | | | | | | |
| | | | | | | | | | | | | | | family B (MDR/TAP), | | | | | | | | | | | | | | |
| | | | | | | | | | | | | | | member 2, ABCB2 | | | | | | | | | | | | | | |
| 404_at | IL4R | X52425 | 6 | Pass | 29 00 | 8 29 | 13 | Pass | TRUE | FALSE | FALSE | 13 62 | 2.13 | interleukin 4 receptor, | 16p11 2- | | | | | | | | | | | | | |
| | | | | | | | | | | | | | | IL4R | 12 1 | | | | | | | | | | | | | |
| 37101_at | DKFZP564 | AL050008 | 6 | Pass | 19.17 | 5 19 | 12 | Pass | TRUE | FALSE | FALSE | 9 00 | 2.13 | DKFZP564A063 protein, | | | | | | | | | | | | | | |
| | | | | | | | | | | | | | | DKFZP564A063 | | | | | | | | | | | | | | |
| 32904_at | PRF1 | M28393 | 6 | Pass | 86 00 | 65 71 | 13 | Pass | TRUE | FALSE | FALSE | 40.38 | 2.13 | perform 1 (preforming | 10q22 | | | | | | | | | | | | | |
| | | | | | | | | | | | | | | protein), PRF1 | | | | | | | | | | | | | | |
| 33228_g_at | IL10RB | AI984234 | 6 | Pass | 55 50 | 17 95 | 13 | Pass | TRUE | FALSE | FALSE | 26.08 | 2.13 | interleukin 10 receptor, | 21q22 11 | | | | | | | | | | | | | |
| | | | | | | | | | | | | | | beta: IL10RB | | | | | | | | | | | | | | |
| 40421_at | PIN1 | U49070 | 6 | Pass | 8 67 | 2 58 | 12 | Pass | TRUE | FALSE | FALSE | 4.08 | 2.12 | protein (peptidyl-polyl- | 19p13 | | | | | | | | | | | | | |
| | | | | | | | | | | | | | | cis/trans isomerase) NIMA | | | | | | | | | | | | | | |
| | | | | | | | | | | | | | | interacting 1, PIN1 | | | | | | | | | | | | | | |
| 661_g_at | F4HB | J02783 | 6 | Pass | 49 17 | 17.06 | 12 | Pass | TRUE | FALSE | FALSE | 23 17 | 2.12 | procollagen-proline, 2- | 17q25 | | | | | | | | | | | | | |
| | | | | | | | | | | | | | | oxoglutarate 4- | | | | | | | | | | | | | | |
| | | | | | | | | | | | | | | dioxygenase (proline 4- | | | | | | | | | | | | | | |
| | | | | | | | | | | | | | | hydroxylase), beta | | | | | | | | | | | | | | |
| | | | | | | | | | | | | | | polypeptide (Protein | | | | | | | | | | | | | | |
| | | | | | | | | | | | | | | disulfide isomerase, | | | | | | | | | | | | | | |
| | | | | | | | | | | | | | | thyroid hormonic binding | | | | | | | | | | | | | | |
| 1563_s_at | TNFRSF1A | M58286 | 6 | Pass | 28.17 | 11 91 | 11 | Pass | TRUE | FALSE | FALSE | 13 27 | 2.12 | tumor necrosis factor | 12p13 2 | | | | | | | | | | | | | |
| | | | | | | | | | | | | | | receptor superfamily, | | | | | | | | | | | | | | |
| | | | | | | | | | | | | | | member 1A; TNFRSF1A | | | | | | | | | | | | | | |
| 330_s_at | TUBA1 | X06956 | 6 | Pass | 39 17 | 9 47 | 13 | Pass | TRUE | FALSE | FALSE | 18.46 | 2.12 | tubulin, alpha 1 (testis | 2q | | | | | | | | | | | | | |
| | | | | | | | | | | | | | | specific), TUBA1 | | | | | | | | | | | | | | |
| 41622_r_at | ZNF266 | AA868898 | 6 | Pass | 6 83 | 1.83 | 9 | Pass | TRUE | FALSE | FALSE | 3.22 | 2.12 | zinc finger protein 266, | | | | | | | | | | | | | | |
| | | | | | | | | | | | | | | ZNF266 | | | | | | | | | | | | | | |
| 286_at | H2AFO | L19779 | 6 | Pass | 61 50 | 25 26 | 13 | Pass | TRUE | FALSE | FALSE | 29 00 | 2.12 | 112A histone family, | | | | | | | | | | | | | | |
| | | | | | | | | | | | | | | member O, H2AFO | | | | | | | | | | | | | | |
| 41282_s_at | PEX10 | AA194159 | 4 | Pass | 11.50 | 3.70 | 7 | Pass | TRUE | FALSE | FALSE | 5.43 | 2.12 | peroxisome biogensis | | | | | | | | | | | | | | |
| | | | | | | | | | | | | | | factor 10, PEX10 | | | | | | | | | | | | | | |
| 41745_at | IFITM3 | X57352 | 6 | Pass | 207.17 | 120 90 | 13 | Pass | TRUE | FALSE | FALSE | 97.85 | 2.12 | interferon induced | | | | | | | | | | | | | | |
| | | | | | | | | | | | | | | transmembrane protein 3 | | | | | | | | | | | | | | |
| | | | | | | | | | | | | | | (1-8U), IFITM3 | | | | | | | | | | | | | | |
| 31845_at | ELF4 | U32645 | 6 | Pass | 18 17 | 7.70 | 12 | Pass | TRUE | FALSE | FALSE | 8 58 | 2.12 | E74-like factor 4 (cis | Xq26 | | | | | | | | | | | | | |
| | | | | | | | | | | | | | | domain transcription | | | | | | | | | | | | | | |
| | | | | | | | | | | | | | | factor), ELF4 | | | | | | | | | | | | | | |
| 1116 at | CD19 | M28170 | 6 | Pass | 6.83 | 1 33 | 13 | Pass | TRUE | FALSE | FALSE | 3 23 | 2.12 | CD19 antigen, CD19 | 16p11 2 | | | | | | | | | | | | | |
| 40688_at | LAT | AJ223280 | 6 | Pass | 27.67 | 4.37 | 12 | Pass | TRUE | FALSE | FALSE | 13.08 | 2.11 | linker for activation of T | | | | | | | | | | | | | | |
| | | | | | | | | | | | | | | cells; LAT | | | | | | | | | | | | | | |
| 162_at | USP11 | U44839 | 6 | Pass | 46.17 | 9.66 | 13 | Pass | TRUE | FALSE | FALSE | 21.85 | 2.11 | ubiquinin specific protease | Xp21.2- | | | | | | | | | | | | | |
| | | | | | | | | | | | | | | 11, USP11 | p11.2 | | | | | | | | | | | | | |
| 34906 g at | UNK AA9 | AA977136 | 8 | Pass | 32 50 | 8 85 | 13 | Pass | TRUE | FALSE | FALSE | 15 38 | 2.11 | | | | | | | | | | | | | | | |
| 31514_at | DOK2 | AF034970 | 6 | Pass | 25 33 | 8 80 | 10 | Pass | TRUE | FALSE | FALSE | 12 00 | 2.11 | docking protein 2, 56kD, | | | | | | | | | | | | | | |
| | | | | | | | | | | | | | | DOK2 | | | | | | | | | | | | | | |
| 37002_at | BLVRB | D32143 | 6 | Pass | 25 00 | 11 47 | 13 | Pass | TRUE | FALSE | FALSE | 11 85 | 2.11 | bilverdin reductase B | 19q13 1- | | | | | | | | | | | | | |
| | | | | | | | | | | | | | | (flavin reductase | q13 2 | | | | | | | | | | | | | |
| | | | | | | | | | | | | | | (NADPH), BLVRB | | | | | | | | | | | | | | |
| 1357_at | USP4 | U20657 | 6 | Pass | 12 33 | 4.59 | 13 | Pass | TRUE | FALSE | FALSE | 5.85 | 2.11 | ubiquinin specific protease | 3p21 3 | | | | | | | | | | | | | |
| | | | | | | | | | | | | | | 4 (proto-oncogene), USP4 | | | | | | | | | | | | | | |
| 32317_s_at | SULT1A2 | U34804 | 6 | Pass | 24.50 | 11.33 | 13 | Pass | TRUE | FALSE | FALSE | 11.62 | 2.11 | sulfotransferase family 1A, | 16p12, 1 | | | | | | | | | | | | | |
| | | | | | | | | | | | | | | phenol-preferring, member | 6p12.1- | | | | | | | | | | | | | |
| | | | | | | | | | | | | | | 2, sulfotransferase family | p11 2 | | | | | | | | | | | | | |
| | | | | | | | | | | | | | | 1A, phenol-preferring, | | | | | | | | | | | | | | |
| | | | | | | | | | | | | | | member 1, | | | | | | | | | | | | | | |
| | | | | | | | | | | | | | | SULT1A2 SULT1A1 | | | | | | | | | | | | | | |
| 35774_4_at | NDUFB7 | AA527880 | 6 | Pass | 24 33 | 7 55 | 13 | Pass | TRUE | FALSE | FASLE | 11 54 | 2.11 | NADH dehydrogenase | | | | | | | | | | | | | | |
| | | | | | | | | | | | | | | (ubiquinone) 1 beta | | | | | | | | | | | | | | |
| | | | | | | | | | | | | | | subcomplex 7 (18kD, | | | | | | | | | | | | | | |
| | | | | | | | | | | | | | | B18): NDUFB7 | | | | | | | | | | | | | | |
| 40870_g_at | RBMG | AF069517 | 6 | Pass | 12 17 | 3 97 | 13 | Pass | TRUE | FALSE | FALSE | 5.77 | 2.11 | RNA binding motif protein | 3p21 3 | | | | | | | | | | | | | |
| | | | | | | | | | | | | | | 6, RBM6 | | | | | | | | | | | | | | |
| 39081 at | MT2A | AI547258 | 6 | Pass | 11.83 | 4 31 | 13 | Pass | TRUE | FALSE | FALSE | 5 62 | 2.11 | metallothionen 2A, | 16q13 | | | | | | | | | | | | | |
| 1375_s_at | TIMP2 | M32304 | 4 | Pass | 14.75 | 8 26 | 9 | Pass | TRUE | FALSE | FALSE | 7.00 | 2.11 | tissue inhibitor of | 17q25 | | | | | | | | | | | | | |
| | | | | | | | | | | | | | | metalloproteinase 2, | | | | | | | | | | | | | | |
| | | | | | | | | | | | | | | TIMP2 | | | | | | | | | | | | | | |
| 36615 at | ARP | M83751 | 4 | Pass | 19 75 | 5.50 | 8 | Pass | TRUE | FALSE | FALSE | 9 38 | 2.11 | Arginine-rich protein, | 3p21 1 | | | | | | | | | | | | | |
| 35312_at | MCM2 | D20163 | 6 | Pass | 17 50 | 3.62 | 13 | Pass | TRUE | FALSE | FALSE | 8 31 | 2.11 | minichromosome | 3q21 | | | | | | | | | | | | | |
| | | | | | | | | | | | | | | maintenance deficient (S | | | | | | | | | | | | | | |
| | | | | | | | | | | | | | | cerevisae) 2 (mitohn | | | | | | | | | | | | | | |
| | | | | | | | | | | | | | | MCM2 | | | | | | | | | | | | | | |
| 37801 at | F16 | AF112972 | 6 | Pass | 9 00 | 3 58 | 11 | Pass | TRUE | FALSE | FALSE | 4 27 | 2.11 | TJ6 protein, F16 | | | | | | | | | | | | | | |
| 38621_at | UNK_AJ0 | AJ012008 | 6 | Pass | 10 00 | 2 45 | 12 | Pass | TRUE | FALSE | FALSE | 4 75 | 2.11 | dimethylargine | 6p21.3 | | | | | | | | | | | | | |
| | | | | | | | | | | | | | | dimethylaminohydrolase 2, | | | | | | | | | | | | | | |
| | | | | | | | | | | | | | | DDAH2 | | | | | | | | | | | | | | |
| 37939 | at | UNK AL0 | AL022318 | 6 | Pass | 45.67 | 11 69 | 13 | Pass | TRUE | FALSE | FALSE | 21 69 | 2.11 | | | | | | | | | | | | | | |
| 1405_r_at | SCYA5 | M21121 | 6 | Pass | 68.17 | 25 93 | 13 | Pass | TRUE | FALSE | FALSE | 32 38 | 2.10 | small inducible cytokine | 17q11 2- | | | | | | | | | | | | | |
| | | | | | | | | | | | | | | AS (RANTES), SCYA5 | q12 | | | | | | | | | | | | | |
| 35150_at | TNFRSF5 | X60592 | 6 | Pass | 28 33 | 7 66 | 13 | Pass | TRUE | FALSE | FALSE | 13 46 | 2.10 | tumor necrosis factor | 20q12- | | | | | | | | | | | | | |
| | | | | | | | | | | | | | | receptor superfamily, | q13.2 | | | | | | | | | | | | | |
| | | | | | | | | | | | | | | member 5, TNFRSF5 | | | | | | | | | | | | | | |
| 34864_at | CGI-57 | AF070638 | 6 | Pass | 38 17 | 12.35 | 13 | Pass | TRUE | FALSE | FALSE | 18.15 | 2.10 | hypothetical protein, GCI- | | | | | | | | | | | | | | |
| | | | | | | | | | | | | | | 57 | | | | | | | | | | | | | | |
| 34861_at | GOLGA3 | D63997 | 6 | Pass | 10 83 | 1.47 | 13 | Pass | TRUE | FALSE | FALSE | 5 15 | 2.10 | golgi autoantigen, golgin | 12 | | | | | | | | | | | | | |
| | | | | | | | | | | | | | | subfanmily 3, GOLGA3 | | | | | | | | | | | | | | |
| 32253_at | ATNIL | AB007927 | 6 | Pass | 31 67 | 4 97 | 13 | Pass | TRUE | FALSE | FALSE | 15.08 | 2.10 | arginine glutamine acid | 1p36 1- | | | | | | | | | | | | | |
| | | | | | | | | | | | | | | dipeptide RE repeats, | p36 2 | | | | | | | | | | | | | |
| | | | | | | | | | | | | | | RERE | | | | | | | | | | | | | | |
| 41264 at | UNK AL0 | AL050172 | 4 | Pass | 6.00 | 2 94 | 7 | Pass | TRUE | FALSE | FALSE | 2 86 | 2.10 | | | | | | | | | | | | | | | |
| 34295_at | USP15 | AB011101 | 6 | Pass | 6.00 | 3 16 | 7 | Pass | TRUE | FALSE | FALSE | 2 86 | 2.10 | ubiquinine specific protease | 12q14 | | | | | | | | | | | | | |
| | | | | | | | | | | | | | | 15, USP15 | | | | | | | | | | | | | | |
| 40780_at | CTBP2 | AF016507 | 6 | Pass | 15 33 | 10.46 | 13 | Pass | TRUE | FALSE | FALSE | 7 31 | 2.10 | C-terminal binding protein | 21q21 3 | | | | | | | | | | | | | |
| | | | | | | | | | | | | | | 2, CTBP2 | | | | | | | | | | | | | | |
| 38453_at | ICAM2 | X15606 | 6 | Pass | 25 50 | 5 36 | 13 | Pass | TRUE | FALSE | FALSE | 12.15 | 2.10 | intercellular adhesion | 17q23- | | | | | | | | | | | | | |
| | | | | | | | | | | | | | | molecule 2, ICAM2 | q25 | | | | | | | | | | | | | |
| 36889_at | FCER1G | M33195 | 6 | Pass | 56 00 | 35.60 | 13 | Pass | TRUE | FALSE | FALSE | 26 69 | 2.10 | Fc fragment of IgH, high | 1q23 | | | | | | | | | | | | | |
| | | | | | | | | | | | | | | affinity 1, receptor for, | | | | | | | | | | | | | | |
| | | | | | | | | | | | | | | gamma polypeptide, | | | | | | | | | | | | | | |
| | | | | | | | | | | | | | | FCER1G | | | | | | | | | | | | | | |
| 38053_{—l s}_at | BRE | AF015767 | 6 | Pass | 9.83 | 3 13 | 13 | Pass | TRUE | FALSE | FALSE | 4 69 | 2.10 | bram and reproductive | | | | | | | | | | | | | | |
| | | | | | | | | | | | | | | organ-expressed | | | | | | | | | | | | | | |
| | | | | | | | | | | | | | | (TNFRSF1A modulator), | | | | | | | | | | | | | | |
| | | | | | | | | | | | | | | BRE | | | | | | | | | | | | | | |
| 32816_at | SGT | AL050156 | 6 | Pass | 6 67 | 2.25 | 11 | Pass | TRUE | FALSE | FALSE | 3 18 | 2.10 | small glutamine-rich | 19p13 | | | | | | | | | | | | | |
| | | | | | | | | | | | | | | tetratricopeptide repeat | | | | | | | | | | | | | | |
| | | | | | | | | | | | | | | (TPR)-containing, SGT | | | | | | | | | | | | | | |
| 36986 at | UNK AL0 | AL031295 | 6 | Pass | 117.17 | 29.18 | 13 | Pass | TRUE | FALSE | FALSE | 55 92 | 2.10 | | | | | | | | | | | | | | | |
| 38121_at | WARS | X59892 | 6 | Pass | 34 17 | 22 83 | 13 | Pass | TRUE | FALSE | FALSE | 16 31 | 2.10 | tryptophanyl-tRNA | 14q23- | | | | | | | | | | | | | |
| | | | | | | | | | | | | | | synthetase, WARS | q31 | | | | | | | | | | | | | |
| 32877 t at | UNK AA5 | AA524802 | 4 | Pass | 49 50 | 23 69 | 11 | Pass | TRUE | FALSE | FALSE | 23 64 | 2.09 | | | | | | | | | | | | | | | |
| 40596_at | TCOF1 | U76366 | 6 | Pass | 22 33 | 4 55 | 12 | Pass | TRUE | FALSE | FALSE | 10.67 | 2.09 | Troacher Collins- | 5q32- | | | | | | | | | | | | | |
| | | | | | | | | | | | | | | Franceschetti syndrome 1, | q33 1 | | | | | | | | | | | | | |
| | | | | | | | | | | | | | | TCOF 1 | | | | | | | | | | | | | | |
| 39072_at | MX11 | L07648 | 6 | Pass | 28.50 | 13.14 | 13 | Pass | TRUE | FALSE | FALSE | 13.62 | 2.09 | MAX-interacting protein | 10q24- | | | | | | | | | | | | | |
| | | | | | | | | | | | | | | 1; MX11 | q25 | | | | | | | | | | | | | |
| 37033_s_at | GPX1 | X13710 | 6 | Pass | 279 17 | 52 77 | 13 | Pass | TRUE | FALSE | FALSE | 133 38 | 2.09 | glutathione perosidase 1, | 3p21 3 | | | | | | | | | | | | | |
| | | | | | | | | | | | | | | GPX1 | | | | | | | | | | | | | | |
| 32195 at | UNK AL0 | AL049450 | 6 | Pass | 14 17 | 5 12 | 13 | Pass | TRUE | FALSE | FALSE | 6 77 | 2.09 | | | | | | | | | | | | | | | |
| 37390_at | KIAA0224 | D86977 | 6 | Pass | 13 67 | 2 94 | 13 | Pass | TRUE | FALSE | FALSE | 6 54 | 2.09 | KIAA0224 gene product, | 16 | | | | | | | | | | | | | |
| | | | | | | | | | | | | | | KIAA0224 | | | | | | | | | | | | | | |
| 38449_at | UNK_W28 | 6 | Pass | 15 67 | 8 94 | 8 | Pass | TRUE | FALSE | FALSE | 7.50 | 2.09 | cukaryotic translation | | | | | | | | | | | | | | | |
| | | | | | | | | | | | | | | initiation factor 3, subunit | | | | | | | | | | | | | | |
| | | | | | | | | | | | | | | 3 (gamma, 40kD), EIF3S3 | | | | | | | | | | | | | | |
| 36187_at | RHN | X13973 | 6 | Pass | 39.50 | 15 10 | 13 | Pass | TRUE | FALSE | FALSE | 18.92 | 2.09 | ribunuclose/angiogenm | 11p15 5 | | | | | | | | | | | | | |
| | | | | | | | | | | | | | | inhibitor, RNH | | | | | | | | | | | | | | |
| 34408 at | RTN2 | AF04222 | 6 | Pass | 12 00 | 3 35 | 8 | Pass | TRUE | FALSE | FALSE | 5 75 | 2.09 | reticulon 2, RTN2 | | | | | | | | | | | | | | |
| 36798_g_at | SPN | J04168 | 6 | Pass | 47 50 | 14.95 | 13 | Pass | TRUE | FALSE | FALSE | 22 77 | 2.09 | sialophorin (gpL115, | 16p11 2 | | | | | | | | | | | | | |
| | | | | | | | | | | | | | | leukosalin, CD43), SPN | | | | | | | | | | | | | | |
| 38412_at | PPPIR11 | U53588 | 6 | Pass | 17 00 | 4 24 | 13 | Pass | TRUE | FALSE | FALSE | 8 15 | 2.08 | protein phosphatase 1, | 6p21.3 | | | | | | | | | | | | | |
| | | | | | | | | | | | | | | regulatory (inhibitor) | | | | | | | | | | | | | | |
| | | | | | | | | | | | | | | subunit 11; PPPIR11 | | | | | | | | | | | | | | |
| 34033_s_at | LILRA2 | AF025531 | 6 | Pass | 33 67 | 18 47 | 13 | Pass | TRUE | FALSE | FALSE | 16 15 | 2.08 | leukocyte mamoglobulin | 19q13 4 | | | | | | | | | | | | | |
| | | | | | | | | | | | | | | like receptor subfamily A | | | | | | | | | | | | | | |
| | | | | | | | | | | | | | | (with TM domain), | | | | | | | | | | | | | | |
| | | | | | | | | | | | | | | member 2-LILRA2 | | | | | | | | | | | | | | |
| 241 g at | SRM | M64231 | 6 | Pass | 16 67 | 3 88 | 12 | Pass | TRUE | FALSE | FALSE | 8 00 | 2.08 | spermidine synthase, SRM | 1p36-p22 | | | | | | | | | | | | | |
| 41316 s at | SAFB | U72355 | 6 | Pass | 20 67 | 5 05 | 13 | Pass | TRUE | FALSE | FALSE | 9 92 | 2.08 | scaffoid attachment factor | 10p13 | | | | | | | | | | | | | |
| | | | | | | | | | | | | | | B SAFB | | | | | | | | | | | | | | |
| 36591_at | TUBA1 | X06956 | 6 | Pass | 114.67 | 24.81 | 13 | Pass | TRUE | FALSE | FALSE | 55 08 | 2.08 | tubilin, alpha 1 (testis | 2q | | | | | | | | | | | | | |
| | | | | | | | | | | | | | | specific UBA1 | | | | | | | | | | | | | | |
| 38830_at | ABCF3 | U66685 | 5 | Pass | 9.20 | 3 77 | 7 | Pass | TRUE | FALSE | FALSE | 4 43 | 2.08 | ATP-binding cassette, sub- | 3q25 1- | | | | | | | | | | | | | |
| | | | | | | | | | | | | | | family f (GCN20), | q25 2 | | | | | | | | | | | | | |
| | | | | | | | | | | | | | | member 3, hypothetical | | | | | | | | | | | | | | |
| | | | | | | | | | | | | | | protein FIJ11198 | | | | | | | | | | | | | | |
| | | | | | | | | | | | | | | ABCF3, FLJ11198 | | | | | | | | | | | | | | |
| 38841_at | GDBR1 | AF068195 | 6 | Pass | 9 00 | 4.52 | 12 | Pass | TRUE | FALSE | FALSE | 4.33 | 2.08 | putative glalblastoma cell | 9 | | | | | | | | | | | | | |
| | | | | | | | | | | | | | | differentiation- | | | | | | | | | | | | | | |
| | | | | | | | | | | | | | | related, putative | | | | | | | | | | | | | | |
| | | | | | | | | | | | | | | glalblastoma cell | | | | | | | | | | | | | | |
| | | | | | | | | | | | | | | differentiation-related | | | | | | | | | | | | | | |
| | | | | | | | | | | | | | | protein, GBDR1, GDBR1 | | | | | | | | | | | | | | |
| 37641_at | MTAP44 | D25915 | 6 | Pass | 11 50 | 7.48 | 13 | Pass | TRUE | FALSE | FALSE | 5 54 | 2.08 | interferon-induced, | 1 | | | | | | | | | | | | | |
| | | | | | | | | | | | | | | hepatitus C-associated | | | | | | | | | | | | | | |
| | | | | | | | | | | | | | | microtubular aggregate | | | | | | | | | | | | | | |
| | | | | | | | | | | | | | | protein (44kD); MTAP44 | | | | | | | | | | | | | | |
| 41838_at | UNK_X99 | X99270 | 6 | Pass | 7 50 | 2.35 | 13 | Pass | TRUE | FALSE | FALSE | 3 62 | 2.07 | Xq28, 2000bp sequence | | | | | | | | | | | | | | |
| | | | | | | | | | | | | | | contg ORF, HSXQ280RF | | | | | | | | | | | | | | |
| 41614_at | KIAA0708 | AB014608 | 6 | Pass | 6 83 | 2 04 | 10 | Pass | TRUE | FALSE | FALSE | 3 30 | 2.07 | KIAA0708 protein, | | | | | | | | | | | | | | |
| | | | | | | | | | | | | | | KIAA0708 | | | | | | | | | | | | | | |
| 448_s_at | MEN1 | U93237 | 6 | Pass | 6.83 | 1 83 | 10 | Pass | TRUE | FALSE | FALSE | 3.30 | 2.07 | multiple endoerne | 11q13 | | | | | | | | | | | | | |
| | | | | | | | | | | | | | | neoplasia 1, MEN1 | | | | | | | | | | | | | | |
| 32140_at | SORL1 | Y08110 | 6 | Pass | 81.00 | 22.91 | 13 | Pass | TRUE | FALSE | FALSE | 39 15 | 2.07 | sotulin-related receptor, | 11q23 2- | | | | | | | | | | | | | |
| | | | | | | | | | | | | | | L(DLR class) A repeats- | q24 2 | | | | | | | | | | | | | |
| | | | | | | | | | | | | | | containing, SORL1 | | | | | | | | | | | | | | |
| 32592_at | KIAA0323 | AB002321 | 6 | Pass | 17.17 | 4 67 | 13 | Pass | TRUE | FALSE | FALSE | 8 31 | 2.07 | KIAA0323 protein, | | | | | | | | | | | | | | |
| | | | | | | | | | | | | | | KIAA0323 | | | | | | | | | | | | | | |
| 36709_at | ITGAX | Y00093 | 6 | Pass | 30.83 | 17.06 | 13 | Pass | TRUE | FALSE | FALSE | 14.92 | 2.07 | integrin, alpha X (antigen | 16q11 2 | | | | | | | | | | | | | |
| | | | | | | | | | | | | | | CD11C (p150), alpha | | | | | | | | | | | | | | |
| | | | | | | | | | | | | | | polypeptide), ITGAX | | | | | | | | | | | | | | |
| 41387_{—l r}_at | KIAA0346 | AB002344 | 6 | Pass | 11 50 | 6.16 | 7 | Pass | TRUE | FALSE | FALSE | 5 57 | 2.06 | KIAA0346 protein, | 17p13 1 | | | | | | | | | | | | | |
| | | | | | | | | | | | | | | KIAA0346 | | | | | | | | | | | | | | |
| 41625_at | TRAP240 | AB01165 | 6 | Pass | 5 83 | 3.19 | 12 | Pass | TRUE | FALSE | FALSE | 2 83 | 2.06 | thyroid hormone receptor- | 17 | | | | | | | | | | | | | |
| | | | | | | | | | | | | | | associated protein, 240 | | | | | | | | | | | | | | |
| | | | | | | | | | | | | | | kDa subunit, TRAP240 | | | | | | | | | | | | | | |
| 35254_at | FLN29 | AB007447 | 6 | Pass | 15 67 | 4 63 | 13 | Pass | TRUE | FALSE | FALSE | 7.62 | 2.06 | TLN29 gene product, | 12q | | | | | | | | | | | | | |
| | | | | | | | | | | | | | | FLN29 | | | | | | | | | | | | | | |
| 38780_at | AKRIA1 | J04794 | 6 | Pass | 28.00 | 8.65 | 13 | Pass | TRUE | FALSE | FALSE | 13.62 | 2.06 | aldo-keto reductase family | 1p33-p32 | | | | | | | | | | | | | |
| | | | | | | | | | | | | | | 1, member A1 (aldehyde | | | | | | | | | | | | | | |
| | | | | | | | | | | | | | | reductase); AKRIA1 | | | | | | | | | | | | | | |
| 40890 at | MTX1 | U46920 | 6 | Pass | 27.83 | 7 55 | 13 | Pass | TRUE | FALSE | FALSE | 13.54 | 2.06 | metaxis 1; MTX1 | 1q21 | | | | | | | | | | | | | |
| 39412_at | ZNFI73 | U09825 | 6 | Pass | 12.33 | 5 57 | 9 | Pass | TRUE | FALSE | FALSE | 6.00 | 2.06 | zinc finger protein 173, | 6p21 3 | | | | | | | | | | | | | |
| | | | | | | | | | | | | | | ZNFI73 | | | | | | | | | | | | | | |
| 332315_g_at | RPMS12 | Y11681 | 6 | Pass | 33 67 | 6 53 | 13 | Pass | TRUE | FALSE | FALSE | 16.38 | 2.05 | ribosomal protein, | 19q13 1 | | | | | | | | | | | | | |
| | | | | | | | | | | | | | | mitochondrial, S12, | | | | | | | | | | | | | | |
| | | | | | | | | | | | | | | RPMS12 | Y11681 | 6 | Pass | 33 67 | 6 53 | 13 | Pass | TRUE | FALSE | FALSE | 16.38 | 2.05 | ribosomal protein, | 19q13 1 |
| | | | | | | | | | | | | | | mitochondrial, S12, | | | | | | | | | | | | | | |
| | | | | | | | | | | | | | | RPMS12 | | | | | | | | | | | | | | |
| 34310_at | APKT | Y00486 | 5 | Pass | 25 40 | 6.27 | 11 | Pass | TRUE | FALSE | FALSE | 12 36 | 2.05 | adenine | 16q24 | | | | | | | | | | | | | |
| | | | | | | | | | | | | | | phosphorbosyltransferase, | | | | | | | | | | | | | | |
| | | | | | | | | | | | | | | APRT | | | | | | | | | | | | | | |
| 33409_at | FKBP2 | AAI58243 | 6 | Pass | 15.00 | 4 65 | 13 | Pass | TRUE | FALSE | FALSE | 7 31 | 2.05 | FK506-binding protein 2 | 11q13 1- | | | | | | | | | | | | | |
| | | | | | | | | | | | | | | (13kD), FKBP2 | q13 3 | | | | | | | | | | | | | |
| 33748_at | KIAA0223 | D86976 | 6 | Pass | 53 50 | 13 14 | 13 | Pass | TRUE | FALSE | FALSE | 26.08 | 2.05 | minor Instocompatibility | 19p13 3 | | | | | | | | | | | | | |
| | | | | | | | | | | | | | | antigen HA-1, KIAA0223 | | | | | | | | | | | | | | |
| 33323 r at | SFN | X57348 | 6 | Pass | 11 50 | 4 72 | 13 | Pass | TRUE | FALSE | FALSE | 5 62 | 2.05 | stratifin, SFN | 1p | | | | | | | | | | | | | |
| 32228_at | ADTAB | AB020706 | 5 | Pass | 19 60 | 6 77 | 7 | Pass | TRUE | FALSE | FALSE | 9 57 | 2.05 | adaptor-related protein | 11 | | | | | | | | | | | | | |
| | | | | | | | | | | | | | | complex 2, alpha 2 | | | | | | | | | | | | | | |
| | | | | | | | | | | | | | | subunit, AP2A2 | | | | | | | | | | | | | | |
| 32236_at | UBE2G2 | AF032456 | 6 | Pass | 25.17 | 11 18 | 13 | Pass | TRUE | FALSE | FALSE | 12.31 | 2.04 | ubiquitin-conjugating | 21q22 3 | | | | | | | | | | | | | |
| | | | | | | | | | | | | | | enzyme E2G 2 | | | | | | | | | | | | | | |
| | | | | | | | | | | | | | | (homologous to yeast | | | | | | | | | | | | | | |
| | | | | | | | | | | | | | | UBC7), UBE2G2 | | | | | | | | | | | | | | |
| 32629_f_at | BTN3A1 | U90552 | 6 | Pass | 111.33 | 28 98 | 13 | Pass | TRUE | FALSE | FALSE | 54.46 | 2.04 | butyrophilin, subfamily 3, | 6p22 1 | | | | | | | | | | | | | |
| | | | | | | | | | | | | | | member A1, BTN3A1 | | | | | | | | | | | | | | |
| 35769_at | GPR56 | AJ011001 | 6 | Pass | 23 17 | 9.30 | 12 | Pass | TRUE | FALSE | FALSE | 11 33 | 2.04 | G protein-coupled receptor | 16q13 | | | | | | | | | | | | | |
| | | | | | | | | | | | | | | 56; GPR56 | | | | | | | | | | | | | | |
| 39541 at | UNK W52 | W52003 | 4 | Pass | 12 00 | 4 08 | 8 | Pass | TRUE | FALSE | FALSE | 5 88 | 2.04 | | | | | | | | | | | | | | | |
| 33898_at | MCRS1 | AF015308 | 6 | Pass | 16 33 | 5 28 | 8 | Pass | TRUE | FALSE | FALSE | 8 00 | 2.04 | microspheride protein 1, | 12 | | | | | | | | | | | | | |
| | | | | | | | | | | | | | | MCRS1 | | | | | | | | | | | | | | |
| 36155_at | KIAA0275 | D87465 | 6 | Pass | 67 83 | 11 11 | 13 | Pass | TRUE | FALSE | FALSE | 33.23 | 2.04 | KIAA0275 gene product, | 10 | | | | | | | | | | | | | |
| | | | | | | | | | | | | | | KIAA0275 | | | | | | | | | | | | | | |
| 709_at | UNK_J003 | J00314 | 6 | Pass | 16 50 | 3 62 | 11 | Pass | TRUE | FALSE | FALSE | 8 09 | 2.04 | tubulin, beta polypeptide | 6p21.3 | | | | | | | | | | | | | |
| | | | | | | | | | | | | | | TUBB | | | | | | | | | | | | | | |
| 37147_at | SCGF | AF020044 | 6 | Pass | 18.33 | 5 85 | 12 | Pass | TRUE | FALSE | FALSE | 9.00 | 2.04 | stem cell growth factor, | 19q13 3 | | | | | | | | | | | | | |
| | | | | | | | | | | | | | | lymphocyte secreted C- | | | | | | | | | | | | | | |
| | | | | | | | | | | | | | | type lectin, SCGF | | | | | | | | | | | | | | |
| 39865 at | UNK AJ89 | AI890903 | 6 | Pass | 9.67 | 2 66 | 12 | Pass | TRUE | FALSE | FALSE | 4 75 | 2.04 | | | | | | | | | | | | | | | |
| 40809_at | RBM6 | AF069517 | 6 | Pass | 9.67 | 2 73 | 12 | Pass | TRUE | FALSE | FALSE | 4 75 | 2.04 | RNA binding mofit protein | 3p21 3 | | | | | | | | | | | | | |
| | | | | | | | | | | | | | | 6 RBM6 | | | | | | | | | | | | | | |
| 35282_{—l r}_at | CD81 | M33680 | 6 | Pass | 77.33 | 22 92 | 13 | Pass | TRUE | FALSE | FALSE | 38 00 | 2.04 | CD81 antigen (target of | 11p15 | | | | | | | | | | | | | |
| | | | | | | | | | | | | | | antiproliferative antibody | | | | | | | | | | | | | | |
| | | | | | | | | | | | | | | D, CD81 | | | | | | | | | | | | | | |
| 39141_at | ABCF1 | AF027302 | 6 | Pass | 12 83 | 4 17 | 13 | Pass | TRUE | FALSE | FALSE | 6 31 | 2.03 | ATP-binding cassette, sub- | 6p21 33 | | | | | | | | | | | | | |
| | | | | | | | | | | | | | | family F (GCN20), | | | | | | | | | | | | | | |
| | | | | | | | | | | | | | | member 1; ABCF1 | | | | | | | | | | | | | | |
| 715_2_at | GGT1 | D87002 | 6 | Pass | 7.67 | 2 07 | 13 | Pass | TRUE | FALSE | FALSE | 3.77 | 2.03 | gamma- | 22q11 1- | | | | | | | | | | | | | |
| | | | | | | | | | | | | | | glutanyltransferase | q11 2, 22q | | | | | | | | | | | | | |
| | | | | | | | | | | | | | | 1, gamma- | 11.23 | | | | | | | | | | | | | |
| | | | | | | | | | | | | | | glutanyltransferse 2, | | | | | | | | | | | | | | |
| 33338_at | STAT1 | M97936 | 6 | Pass | 20.00 | 9 80 | 12 | Pass | TRUE | FALSE | FALSE | 9 83 | 2.03 | signal transducer and | 2q32 2 | | | | | | | | | | | | | |
| | | | | | | | | | | | | | | activator of transcription 1, | | | | | | | | | | | | | | |
| | | | | | | | | | | | | | | 91kD, STAT1 | | | | | | | | | | | | | | |
| 38257_at | NDUFS8 | AF038406 | 6 | Pass | 10 17 | 5.56 | 10 | Pass | TRUE | FALSE | FALSE | 5 00 | 2.03 | NADH dehydrogenase | 11q13 | | | | | | | | | | | | | |
| | | | | | | | | | | | | | | (ubiquinone) Fe-S protein | | | | | | | | | | | | | | |
| | | | | | | | | | | | | | | 8 (23kD) (NADH- | | | | | | | | | | | | | | |
| | | | | | | | | | | | | | | coenzyme Q reductase), | | | | | | | | | | | | | | |
| 32553_at | MAZ | M94046 | 6 | Pass | 96 33 | 20.40 | 13 | Pass | TRUE | FALSE | FALSE | 47.38 | 2.03 | MYC-associated zinc | 16p11 2 | | | | | | | | | | | | | |
| | | | | | | | | | | | | | | finger protein (purine- | | | | | | | | | | | | | | |
| | | | | | | | | | | | | | | binding transcription | | | | | | | | | | | | | | |
| 1404_r_at | SCYA5 | M21121 | 6 | Pass | 10.50 3 94 | 12 | Pass | TRUE | FALSE | FALSE | 5.17 | 2.03 | small inducible cytokine | 17q11 2- | | | | | | | | | | | | | | |
| | | | | | | | | | | | | | | A5 (RANTES), SCYA5 | q12 | | | | | | | | | | | | | |
| 37256 at | UNK AI82 | AI829890 | 6 | Pass | 10.83 | 2 86 | 12 | Pass | TRUE | FALSE | FALSE | 5 33 | 2.03 | | | | | | | | | | | | | | | |
| 40494_at | DEDD | AF043733 | 6 | Pass | 21.67 | 9.05 | 12 | Pass | TRUE | FALSE | FALSE | 10.67 | 2.03 | death effector domain- | | | | | | | | | | | | | | |
| | | | | | | | | | | | | | | containing; DEDD | | | | | | | | | | | | | | |
| 38864 | UNK W26 | W26851 | 6 | Pass | 13 00 | 2 00 | 10 | Pass | TRUE | FALSE | FALSE | 6 40 | 2.03 | | | | | | | | | | | | | | | |
| 41267_at | KIAA1049 | AB028972 | 6 | Pass | 22 33 | 6.95 | 13 | Pass | TRUE | FALSE | FALSE | 11 00 | 2.03 | KIAA1049 protein, | 16 | | | | | | | | | | | | | |
| | | | | | | | | | | | | | | KIAA1049 | | | | | | | | | | | | | | |
| 32080_at | TETRAN | L11669 | 6 | Pass | 42 17 | 12 22 | 13 | Pass | TRUE | FALSE | FALSE | 20 77 | 2.03 | tetracycline transporter- | 4p16 3 | | | | | | | | | | | | | |
| | | | | | | | | | | | | | | like protein, TETRAN | | | | | | | | | | | | | | |
| 35944_at | UNK_AL0 | AL031228 | 5 | Pass | 14 20 | 5.54 | 7 | Pass | TRUE | FALSE | FALSE | 7.00 | 2.03 | Cluster Incl AL031228, | 6p21 3 | | | | | | | | | | | | | |
| | | | | | | | | | | | | | | Human DNA sequence | | | | | | | | | | | | | | |
| | | | | | | | | | | | | | | from clone 103B10 on | | | | | | | | | | | | | | |
| | | | | | | | | | | | | | | chromosome 6p21 2- | | | | | | | | | | | | | | |
| | | | | | | | | | | | | | | 21 31 Contains the | | | | | | | | | | | | | | |
| | | | | | | | | | | | | | | BING5 gene, exons 11 to | | | | | | | | | | | | | | |
| | | | | | | | | | | | | | | 15 of the BING4 gene, the | | | | | | | | | | | | | | |
| | | | | | | | | | | | | | | gene for GalT3 (beta3- | | | | | | | | | | | | | | |
| | | | | | | | | | | | | | | Galactosyltransferase), the | | | | | | | | | | | | | | |
| | | | | | | | | | | | | | | RPS18 (40S ribosomal | | | | | | | | | | | | | | |
| | | | | | | | | | | | | | | protein S18) gene, the | | | | | | | | | | | | | | |
| | | | | | | | | | | | | | | SACM2L (suppressor of | | | | | | | | | | | | | | |
| | | | | | | | | | | | | | | actin mutation 2, yeast, | | | | | | | | | | | | | | |
| | | | | | | | | | | | | | | homolog) gene, a | | | | | | | | | | | | | | |
| | | | | | | | | | | | | | | pseudogene similar to | | | | | | | | | | | | | | |
| | | | | | | | | | | | | | | TAI-SF1, a Pseudogene | | | | | | | | | | | | | | |
| | | | | | | | | | | | | | | similar to zinz finger | | | | | | | | | | | | | | |
| | | | | | | | | | | | | | | genes, the RING1 gene, | | | | | | | | | | | | | | |
| | | | | | | | | | | | | | | the gene for HKE6 | | | | | | | | | | | | | | |
| | | | | | | | | | | | | | | (RING2), the gene for | | | | | | | | | | | | | | |
| | | | | | | | | | | | | | | HKE4 (RING5), the | | | | | | | | | | | | | | |
| | | | | | | | | | | | | | | RXRB (Retinoid X | | | | | | | | | | | | | | |
| | | | | | | | | | | | | | | receptor beta) gene, the | | | | | | | | | | | | | | |
| | | | | | | | | | | | | | | COL11A2 (collagene, type | | | | | | | | | | | | | | |
| | | | | | | | | | | | | | | XI, alpha 2) gene, the | | | | | | | | | | | | | | |
| | | | | | | | | | | | | | | HI A-DPB2 pseudogene | | | | | | | | | | | | | | |
| | | | | | | | | | | | | | | and part of the HLA- | | | | | | | | | | | | | | |
| | | | | | | | | | | | | | | DPA3 pseudogene | | | | | | | | | | | | | | |
| | | | | | | | | | | | | | | Contains predicted CpG | | | | | | | | | | | | | | |
| 33388 at | UNK AL0 | AL080223 | 6 | Pass | 22 00 | 5 40 | 13 | Pass | TRUE | FALSE | FALSE | 10 85 | 2.03 | | | | | | | | | | | | | | | |
| 40787 at | UNK U90 | U90911 | 6 | Pass | 12 17 | 3 66 | 13 | Pass | TRUE | FALSE | FALSE | 6 00 | 2.03 | | | | | | | | | | | | | | | |
| 35911_r_at | MMPL1 | AJ003147 | 6 | Pass | 24 50 | 8 14 | 12 | Pass | TRUE | FALSE | FALSE | 12 08 | 2.03 | matrix, metalloprotenase- | 16p13 3 | | | | | | | | | | | | | |
| | | | | | | | | | | | | | | like 1, MMPL1 | | | | | | | | | | | | | | |
| 37003_at | CD63 | X62654 | 6 | Pass | 29.00 | 14.71 | 13 | Pass | TRUE | FALSE | FALSE | 14 31 | 2.03 | CD63 antigen (melanoma | 12q12- | | | | | | | | | | | | | |
| | | | | | | | | | | | | | | 1 antigen), CD63 | q13 | | | | | | | | | | | | | |
| 38475_at | DCTN-50 | U50733 | 6 | Pass | 14.50 | 5.54 | 13 | Pass | TRUE | FALSE | FALSE | 7.15 | 2.03 | dynanitin (dynactin | 12 | | | | | | | | | | | | | |
| | | | | | | | | | | | | | | complex 50 kD subunit), | | | | | | | | | | | | | | |
| | | | | | | | | | | | | | | DCTN-50 | | | | | | | | | | | | | | |
| 870_f_at | MT3 | M93311 | 6 | Pass | 31 50 | 13.44 | 11 | Pass | TRUE | FALSE | FALSE | 15 55 | 2.03 | metallothionen 3 (growth | 16q13 | | | | | | | | | | | | | |
| | | | | | | | | | | | | | | inhibitory factor | | | | | | | | | | | | | | |
| | | | | | | | | | | | | | | (neurotrophic)), MT3 | | | | | | | | | | | | | | |
| 34178_at | UNK_AI88 | AI884738 | 6 | Pass | 7.17 | 1 60 | 13 | Pass | TRUE | FALSE | FALSE | 3.54 | 2.03 | zinc finger protein 297, | 6p21.3 | | | | | | | | | | | | | |
| | | | | | | | | | | | | | | ZNF297 | | | | | | | | | | | | | | |
| 36167_at | ATP6F | D89052 | 6 | Pass | 64.50 | 29.51 | 13 | Pass | TRUE | FALSE | FALSE | 31.85 | 2.03 | ATPase, H+transporting, | 1p32 3 | | | | | | | | | | | | | |
| | | | | | | | | | | | | | | lysosomal (vacuolar | | | | | | | | | | | | | | |
| | | | | | | | | | | | | | | proton pump) 21kD, | | | | | | | | | | | | | | |
| 37911_at | STX4A | U07158 | 6 | Pass | 13 33 | 3 88 | 12 | Pass | TRUE | FALSE | FALSE | 6 58 | 2.03 | syntaxin 4A (placental), | | | | | | | | | | | | | | |
| | | | | | | | | | | | | | | STX4A | | | | | | | | | | | | | | |
| 38523_f_at | U2AFIRS2 | D49677 | 6 | Pass | 13.67 | 2 58 | 12 | Pass | TRUE | FALSE | FALSE | 6.75 | 2.02 | U2 small nuclear | Xp22 1 | | | | | | | | | | | | | |
| | | | | | | | | | | | | | | ribonucleoprotein auxiliary | | | | | | | | | | | | | | |
| | | | | | | | | | | | | | | factor, small subunit 2, | | | | | | | | | | | | | | |
| | | | | | | | | | | | | | | U2AFIRS2 | | | | | | | | | | | | | | |
| 37931_at | CENPB | X05299 | 6 | Pass | 10.67 | 2 42 | 11 | Pass | TRUE | FALSE | FALSE | 5 27 | 2.02 | centromere protein B | 20p13 | | | | | | | | | | | | | |
| | | | | | | | | | | | | | | (80kD), CENPB | | | | | | | | | | | | | | |
| 35124_at | ALOX12 | M62982 | 6 | Pass | 14 00 | 4 69 | 13 | Pass | TRUE | FALSE | FALSE | 6.92 | 2.02 | arachidonate 12- | 17p13 1 | | | | | | | | | | | | | |
| | | | | | | | | | | | | | | lipoxygenase, ALOX12 | | | | | | | | | | | | | | |
| 32523_at | CLTB | M20470 | 6 | Pass | 5.17 | 1.83 | 9 | Pass | TRUE | FALSE | FALSE | 2.56 | 2.02 | clathrin, light polypeptide | 4q2-q3 | | | | | | | | | | | | | |
| | | | | | | | | | | | | | | (Leb); CLTB | | | | | | | | | | | | | | |
| 37442 at | UNK AL0 | AL050378 | 6 | Pass | 18.00 | 6 32 | 13 | Pass | TRUE | FALSE | FALSE | 8 92 | 2.02 | | | | | | | | | | | | | | | |
| 40470_at | OGDH | D10523 | 5 | Pass | 14.40 | 3.85 | 7 | Pass | TRUE | FALSE | FALSE | 7.14 | 2.02 | oxoglutarate | 7p14-p3 | | | | | | | | | | | | | |
| | | | | | | | | | | | | | | dehydrogenase | | | | | | | | | | | | | | |
| 37759_at | LAPTM5 | U51240 | 6 | Pass | 229 50 | 56 06 | 13 | Pass | TRUE | FALSE | FALSE | 113 85 | 2.02 | Lysosomal-associated | 1p34 | | | | | | | | | | | | | |
| | | | | | | | | | | | | | | multispanning membrane | | | | | | | | | | | | | | |
| | | | | | | | | | | | | | | protein-5, LAPTM5 | | | | | | | | | | | | | | |
| 37126_at | SSA1 | M62800 | 5 | Pass | 15 00 | 5.83 | 9 | Pass | TRUE | FALSE | FALSE | 7 44 | 2.01 | Syogren syndrome antigen | 11p15 5 | | | | | | | | | | | | | |
| | | | | | | | | | | | | | | A1 (52kD, | | | | | | | | | | | | | | |
| | | | | | | | | | | | | | | ribonucleoprotein | | | | | | | | | | | | | | |
| | | | | | | | | | | | | | | autoantigen SS-A/Ro); | | | | | | | | | | | | | | |
| | | | | | | | | | | | | | | SSA1 | | | | | | | | | | | | | | |
| 34260_at | KIAA0683 | 5 | Pass | 6 80 | 2 49 | 8 | Pass | TRUE | FALSE | FALSE | 3 38 | 2.01 | KIAA0683 gene product, | 16 | | | | | | | | | | | | | | |
| | | | | | | | | | | | | | | KIAA0683 | | | | | | | | | | | | | | |
| 1468_at | TRAP1 | U12595 | 6 | Pass | 11.00 | 2.10 | 13 | Pass | TRUE | FALSE | FALSE | 5.46 | 2.01 | heat shock protein 75, | 16 | | | | | | | | | | | | | |
| | | | | | | | | | | | | | | TRAP1 | | | | | | | | | | | | | | |
| 37272_at | ITPKB | X57206 | 6 | Pass | 28 50 | 6 38 | 13 | Pass | TRUE | FALSE | FALSE | 14 15 | 2.01 | unositol 1,4,5- | 1q41-q43 | kinase | | | | | | | | | | | | |
| | | | | | | | | | | | | | | trisphosphate 3-kinase B | | | | | | | | | | | | | | |
| 1830_s_at | TGFB1 | M38449 | 6 | Pass | 30 67 | 6 80 | 13 | Pass | TRUE | FALSE | FALSE | 15 23 | 2.01 | transforming growth | 19q13 1 | | | | | | | | | | | | | |
| | | | | | | | | | | | | | | factor, beta 1, TGFB1 | | | | | | | | | | | | | | |
| 35685_at | RING1 | Z14000 | 6 | Pass | 15 33 | 3.93 | 13 | Pass | TRUE | FALSE | FALSE | 7.62 | 2.01 | ring finger protein 1, | 6p21.3 | | | | | | | | | | | | | |
| | | | | | | | | | | | | | | KING1 | | | | | | | | | | | | | | |
| 1295_at | RELA | L19067 | 6 | Pass | 39.17 | 15 38 | 13 | Pass | TRUE | FALSE | FALSE | 19 46 | 2.01 | v-rel avian | 11q13 | | | | | | | | | | | | | |
| | | | | | | | | | | | | | | reticuloendotheliosis | viral | | | | | | | | | | | | | |
| | | | | | | | | | | | | | | oncogene homolog A | | | | | | | | | | | | | | |
| | | | | | | | | | | | | | | (nuclear factor of kappa | | | | | | | | | | | | | | |
| | | | | | | | | | | | | | | light polypepode gene | | | | | | | | | | | | | | |
| | | | | | | | | | | | | | | enhancer in B-cells 3 | | | | | | | | | | | | | | |
| | | | | | | | | | | | | | | (p65)), RELA | | | | | | | | | | | | | | |
| 32271 at | FOSL1 | X16707 | 4 | Pass | 9 25 | 2 75 | 10 | Pass | TRUE | FALSE | FALSE | 4 60 | 2.01 FOS-like antigen-1, | 11q13 | | | | | | | | | | | | | | |
| 34435 at | AQP9 | AB008775 | 6 | Pass | 7 83 | 5 19 | 16 | Pass | TRUE | FALSE | FALSE | 3 90 | 2.01 | aquaporin 9, AQP9 | | | | | | | | | | | | | | |
| 37647_ | AOAH | M62840 | 6 | Pass | 25 00 | 14 34 | 13 | Pass | TRUE | FALSE | FALSE | 12.46 | 2.01 | acyloxyacyl hydrolase | 7p14-p12 | | | | | | | | | | | | | |
| | | | | | | | | | | | | | | neutrophil), AOAH | | | | | | | | | | | | | | |
| 32611_at | PBP | X75252 | 6 | Pass | 14.50 | 5.09 | 13 | Pass | TRUE | FALSE | FALSE | 7.23 | 2.01 | prostatic binding protein, | | | | | | | | | | | | | | |
| | | | | | | | | | | | | | | PBP | | | | | | | | | | | | | | |
| 2035_s_at | MPE1 | M55914 | 6 | Pass | 125 67 | 55 44 | 13 | Pass | TRUE | FALSE | FALSE | 62 69 | 2.00 | MYC promoter-binding | 1p36 3- | | | | | | | | | | | | | |
| | | | | | | | | | | | | | | protein 1, enolase 1, | p36 2,1pte | | | | | | | | | | | | | |
| | | | | | | | | | | | | | | alpha), ENO1,MPB1 | t-p35 | | | | | | | | | | | | | |
| 37012_at | CAPZB | U03271 | 6 | Pass | 65 83 | 19 11 | 13 | Pass | TRUE | FALSE | FALSE | 32.85 | 2.00 | capping protein (actin | 1p36.1 | | | | | | | | | | | | | |
| | | | | | | | | | | | | | | filament) muscle Z-line, | | | | | | | | | | | | | | |
| | | | | | | | | | | | | | | beta, CAPZB | | | | | | | | | | | | | | |
| 34830 at | UNK W25 | W25986 | 6 | Pass | 36 83 | 7 31 | 13 | Pass | TRUE | FALSE | FALSE | 18 38 | 2.00 | | | | | | | | | | | | | | | |
| 38398_at | MADD | AB002356 | 6 | Pass | 16 33 | 6 09 | 13 | Pass | TRUE | FALSE | FALSE | 8 15 | 2.00 | MAP-kinase activating | 11p11 2 | | | | | | | | | | | | | |
| | | | | | | | | | | | | | | death domain: MADD | | | | | | | | | | | | | | |
| 40143_at | KIAA0140 | D50930 | 6 | Pass | 18.33 | 7 23 | 13 | Pass | TRUE | FALSE | FALSE | 9 15 | 2.00 | KIAA0140 gene product, | | | | | | | | | | | | | | |
| | | | | | | | | | | | | | | KIAA0140 | | | | | | | | | | | | | | |
| 35205_at | DKFZP580 | AL050280 | 6 | Pass | 32.50 | 9.40 | 13 | Pass | TRUE | FALSE | FALSE | 16.23 | 2.00 | DKFZP586B0519 protein, | | | | | | | | | | | | | | |
| | | | | | | | | | | | | | | DKFZP586B0519 | | | | | | | | | | | | | | |
| 1427 g at | SLA | D89077 | 6 | Pass | 22.33 | 10 06 | 13 | Pass | TRUE | FALSE | FALSE | 11 15 | 2.00 | Src-like-adapter, SLA | 8q24 | | | | | | | | | | | | | |
| 41800_s_at | TTC2 | U46571 | 6 | Pass | 22.33 | 5 65 | 13 | Pass | TRUE | FALSE | FALSE | 11 15 | 2.00 tetracopeptide repeat | 17q11 2 | | | | | | | | | | | | | | |
| | | | | | | | | | | | | | | domain 2, TTC2 | | | | | | | | | | | | | | |
| 39149_at | PRCC | X90720 | 6 | Pass | 10.00 | 2 00 | 7 | Pass | TRUE | FALSE | FALSE | 5 00 | 2.00 | papillary renal cell | 1q21 1 | | | | | | | | | | | | | |
| | | | | | | | | | | | | | | carcinoma (translocation- | | | | | | | | | | | | | | |
| | | | | | | | | | | | | | | associated), PRCC | | | | | | | | | | | | | | |
| 4I778_at | SLCIA5 | U53347 | 5 | Pass | 8.00 | 3.61 | 8 | Pass | TRUE | FALSE | FALSE | 4.00 | 2.80 | solute carrier family 1 | 19q13 3 | | | | | | | | | | | | | |
| | | | | | | | | | | | | | | (neutral amino acid | | | | | | | | | | | | | | |
| | | | | | | | | | | | | | | transporter), member 5, | | | | | | | | | | | | | | |
| | | | | | | | | | | | | | | SLCIA5 | | | | | | | | | | | | | | |
| 40149_at | DKFZP547 | AL049924 | 6 | Pass | 7.00 | 0.63 | 8 | Pass | TRUE | FALSE | FALSE | 3.50 | 2.00 | SII2-B homolog, | | | | | | | | | | | | | | |
| | | | | | | | | | | | | | | DKFZP547G1110 | | | | | | | | | | | | | | |
| 453_at | SMARCC2 | U6616 | 6 | Pass | 5.00 | 2.00 | 8 | Pass | TRUE | FALSE | FALSE | 2.50 | 2.00 | SWI/SNF related, matrix | 12q13- | | | | | | | | | | | | | |
| | | | | | | | | | | | | | | asociated, actin dependent | q14 | | | | | | | | | | | | | |
| | | | | | | | | | | | | | | regulator of chromatin, | | | | | | | | | | | | | | |
| | | | | | | | | | | | | | | subfamily c, member 2, | | | | | | | | | | | | | | |
| | | | | | | | | | | | | | | SMARCC2 | | | | | | | | | | | | | | |
| 40840_at | PPIF | M80254 | 6 | Pass | 8 67 | 2.66 | 9 | Pass | TRUE | FALSE | FALSE | 4 33 | 2.00 | peptdylpolyl isomerase F | 10q22- | | | | | | | | | | | | | |
| | | | | | | | | | | | | | | (cyclophlin F), PPIF | q23 | | | | | | | | | | | | | |
| 1014_at | POLG | U60325 | 6 | Pass | 10 00 | 3.41 | 12 | Pass | TRUE | FALSE | FALSE | 5.00 | 2.00 | polymerase (DNA | 15q25 | | | | | | | | | | | | | |
| | | | | | | | | | | | | | | directed, gamma, POLG | | | | | | | | | | | | | | |
| 1243_at | DDB2 | U18300 | 6 | Pass | 11.00 | 1.79 | 12 | Pass | TRUE | FALSE | FALSE | 5 50 | 2.00 | damage-specific DNA | 11p12- | | | | | | | | | | | | | |
| | | | | | | | | | | | | | | binding protein 2 (48kD); | p11 | | | | | | | | | | | | | |
| | | | | | | | | | | | | | | DDB2 | | | | | | | | | | | | | | |
| 1487_at | ESRRA | L38487 | 6 | Pass | 12 00 | 5 18 | 12 | Pass | TRUE | FALSE | FALSE | 6 00 | 2.00 | estrogen-related receptor | 11q12 | | | | | | | | | | | | | |
| | | | | | | | | | | | | | | alpha, ESRRA | | | | | | | | | | | | | | |
| 197_at | NME3 | U29656 | 6 | Pass | 10 50 | 2.88 | 12 | Pass | TRUE | FALSE | FALSE | 5 25 | 2.00 | non-metastatic cells 1, | 16q13 | | | | | | | | | | | | | |
| | | | | | | | | | | | | | | protein expressed in, | | | | | | | | | | | | | | |
| | | | | | | | | | | | | | | NME3 | | | | | | | | | | | | | | |
| 32533_s_at | VAMP5 | AF054825 | 6 | Pass | 9 50 | 4.23 | 12 | Pass | TRUE | FALSE | FALSE | 4 75 | 2.00 | vesicle-associated | | | | | | | | | | | | | | |
| | | | | | | | | | | | | | | membrane protein 5 | | | | | | | | | | | | | | |
| | | | | | | | | | | | | | | (myobrevin), VAMP5 | | | | | | | | | | | | | | |
| 34871 at | UNK W30 | W30677 | 6 | Pass | 18 67 | 6 83 | 12 | Pass | TRUE | FALSE | FALSE | 9 33 | 2.00 | | | | | | | | | | | | | | | |
| 36815 at | UNK AF0 | AF038185 | 6 | Pass | 16 00 | 3.41 | 12 | Pass | TRUE | FALSE | FALSE | 8 00 | 2.00 | | | | | | | | | | | | | | | |
| 40282 s at | DF | M84526 | 6 | Pass | 38 00 | 25 78 | 13 | Pass | TRUE | FALSE | FALSE | 19 00 | 2.00 | D component of | 19 | | | | | | | | | | | | | |
| | | | | | | | | | | | | | | complement (adipsin), DF | | | | | | | | | | | | | | |
| 36645_at | RELA | L19067 | 6 | Pass | 33 67 | 12.82 | 13 | Pass | TRUE | FALSE | FALSE | 16 85 | 2.00 | v-rel avian | 11q13 | | | | | | | | | | | | | |
| | | | | | | | | | | | | | | reticuloendotheliosis viral | | | | | | | | | | | | | | |
| | | | | | | | | | | | | | | oncogene homolog A | | | | | | | | | | | | | | |
| | | | | | | | | | | | | | | (nuclear factor af kappa | | | | | | | | | | | | | | |
| | | | | | | | | | | | | | | light polypeptide gene | | | | | | | | | | | | | | |
| | | | | | | | | | | | | | | enhancer in B-cells 3 | | | | | | | | | | | | | | |
| | | | | | | | | | | | | | | (p65)), RELA | | | | | | | | | | | | | | |
| 41471_at | SI00A9 | W72424 | 6 | Pass | 313 50 | 166.70 | 13 | Pass | TRUE | FALSE | FALSE | 157 00 | 2.00 | S100 calcium-binding | 1q21 | | | | | | | | | | | | | |
| | | | | | | | | | | | | | | protein A9 (calgranulin B), | | | | | | | | | | | | | | |
| | | | | | | | | | | | | | | S100A9 | | | | | | | | | | | | | | |
| 33227_at | IL10RB | AI984234 | 6 | Pass | 15.33 | 6 22 | 13 | Pass | TRUE | FALSE | FALSE | 7 69 | 1.99 | interleukin 10 receptor, | 21q22.11 | | | | | | | | | | | | | |
| | | | | | | | | | | | | | | beta, IL10RB | | | | | | | | | | | | | | |
| 58308 g at | KIAA0607 | AB0111179 | 6 | Pass | 30 50 | 9 31 | 13 | Pass | TRUE | FALSE | FALSE | 15 31 | 1.99 | neurochondrium, KIAA0607 | 1 | | | | | | | | | | | | | |
| 36145_at | SIAHBP1 | U51586 | 6 | Pass | 22 50 | 6.77 | 13 | Pass | TRUE | FALSE | FALSE | 11 31 | 1.99 | siah binding protein 1, | 8q24 2- | | | | | | | | | | | | | |
| | | | | | | | | | | | | | | PEP interacting repression, | qtel | | | | | | | | | | | | | |
| | | | | | | | | | | | | | | pyrimidine heat binding | | | | | | | | | | | | | | |
| | | | | | | | | | | | | | | splicing factor, Ro | | | | | | | | | | | | | | |
| | | | | | | | | | | | | | | ribonucleoprotein-binding | | | | | | | | | | | | | | |
| | | | | | | | | | | | | | | protein 1, SIAHBP1 | | | | | | | | | | | | | | |
| 40469_at | MCM3AP | AB011144 | 6 | Pass | 22 50 | 6 66 | 13 | Pass | TRUE | FALSE | FALSE | 11 31 | 1.99 | aminochromosome | 21q22 3 | | | | | | | | | | | | | |
| | | | | | | | | | | | | | | maintenance deficient (S | | | | | | | | | | | | | | |
| | | | | | | | | | | | | | | cerevisiae) 3-associated | | | | | | | | | | | | | | |
| | | | | | | | | | | | | | | protein, MCM3AP | | | | | | | | | | | | | | |
| 1506_at | IL2RG | D110086 | Pass | 26 33 | 8.02 | 12 | Pass | TRUE | FALSE | FALSE | 13 25 | 1.99 | interleukin 2 receptor, | Xq13 1 | | | | | | | | | | | | | | |
| | | | | | | | | | | | | | | gamma (severe combined | | | | | | | | | | | | | | |
| | | | | | | | | | | | | | | immunodeficiency). | | | | | | | | | | | | | | |
| 33781_s_at | UBE2M | AF075599 | 6 | Pass | 6.50 | 2 43 | 11 | Pass | TRUE | FALSE | FALSE | 3 27 | 1.99 | ubiquitin-conjugating | | | | | | | | | | | | | | |
| | | | | | | | | | | | | | | enzyme L2M (homologous | | | | | | | | | | | | | | |
| | | | | | | | | | | | | | | to yeast UBC12), UBE2M | | | | | | | | | | | | | | |
| 40501_s_at | MYBPC1 | X73114 | 5 | Pass | 14 20 | 4 44 | 13 | Pass | TRUE | FALSE | FALSE | 7 15 | 1.90 | myosin-binding protein C, | 12 | | | | | | | | | | | | | |
| | | | | | | | | | | | | | | slo-type, MYBPC1 | | | | | | | | | | | | | | |
| 35267_g_at | BC10 | AL049288 | 6 | Pass | 14 33 | 4.93 | 13 | Pass | TRUE | FALSE | FALSE | 7 23 | 1.98 | bladder cancer associated | 20 | | | | | | | | | | | | | |
| | | | | | | | | | | | | | | protein, BLCAP | | | | | | | | | | | | | | |
| 36959_at | UBE2V1 | U49278 | 6 | Pass | 25.00 | 9.98 | 13 | Pass | TRUE | FALSE | FALSE | 12.62 | 1.98 | hypothetical protein | 20q13 2 | | | | | | | | | | | | | |
| | | | | | | | | | | | | | | DKFZp547H084,ubiquitin- | | | | | | | | | | | | | | |
| | | | | | | | | | | | | | | conjugating enzyme E2 | | | | | | | | | | | | | | |
| | | | | | | | | | | | | | | variant 1; | | | | | | | | | | | | | | |
| | | | | | | | | | | | | | | DKFZp547H084, UBF2V1 | | | | | | | | | | | | | | |
| 32967_at | TOSO | AF057557 | 6 | Pass | 23 00 | 7 46 | 13 | Pass | TRUE | FALSE | FALSE | 11 62 | 1.98 | regulator of Fas-induced | | | | | | | | | | | | | | |
| | | | | | | | | | | | | | | apoptosis, TOSO | | | | | | | | | | | | | | |
| 38538_at | RRAS | AI201108 | 6 | Pass | 15.83 | 5.53 | 12 | Pass | TRUE | FALSE | FALSE | 8.00 | 1.98 | related RAS viral (r-ras) | 19q13 3- | | | | | | | | | | | | | |
| | | | | | | | | | | | | | | oncogene homolog; RRAS | qter | | | | | | | | | | | | | |
| 34892_at | TNFRSF10 | AF016266 | 6 | Pass | 15 83 | 5.49 | 13 | Pass | TRUE | FALSE | FALSE | 8 00 1.98 | tumor necrosis factor | 8q22-p21 | | | | | | | | | | | | | | |
| | | | | | | | | | | | | | | receptor superfamily, | | | | | | | | | | | | | | |
| | | | | | | | | | | | | | | member 10b; TNFR5F10B | | | | | | | | | | | | | | |
| 38181_at | MMP11 | X57766 | 6 | Pass | 11 33 | 4 41 | 11 | Pass | TRUE | FALSE | FALSE | 5 73 | 1.98 | matrix metalloprotease | 22q11 23 | | | | | | | | | | | | | |
| | | | | | | | | | | | | | | 11 (stromelysin 3), | | | | | | | | | | | | | | |
| 31826_at | KIAA0674 | AB014574 | 6 | Pass | 28 00 | 9.40 | 13 | Pass | TRUE | FALSE | FALSE | 14 15 | 1.98 | KIAA0674 protein, | 9 | | | | | | | | | | | | | |
| | | | | | | | | | | | | | | KIAA0674 | | | | | | | | | | | | | | |
| 34347_at | DKFZP564 | AL049955 | 6 | Pass | 22 67 | 5 24 | 13 | Pass | TRUE | FALSE | FALSE | 11 46 | 1.98 | DKFZP564J0123 protein, | 3p21.2- | | | | | | | | | | | | | |
| | | | | | | | | | | | | | | DKFZP564J0123 | 24 2 | | | | | | | | | | | | | |
| 40076_at | TPD52L2 | AF004430 | 6 | Pass | 22 67 | 11 27 | 13 | Pass | TRUE | FALSE | FALSE | 11 46 | 1.98 | tumor protein D52-like 2, | 20q13 2- | | | | | | | | | | | | | |
| | | | | | | | | | | | | | | TPD52L2 | | | | | | | | | | | | | | |
| 37967_at | D6S49E | AF000424 | 6 | Pass | 100 17 | 51 60 | 13 | Pass | TRUE | FALSE | FALSE | 50.69 | 1.98 | DNA segment on | 6p21.3 | | | | | | | | | | | | | |
| | | | | | | | | | | | | | | chromosome 6 (unique) 49 | | | | | | | | | | | | | | |
| | | | | | | | | | | | | | | expressed sequence; | | | | | | | | | | | | | | |
| | | | | | | | | | | | | | | D6S49E Homo sapiens | | | | | | | | | | | | | | |
| | | | | | | | | | | | | | | LST1 mRNA | | | | | | | | | | | | | | |
| 37487_at | KIAA1093 | AB029016 | 6 | Pass | 6 | Pass | 10 33 | 3 88 | 13 | Pass | TRUE | FALSE | FALSE | 5 23 | 1.98 | KIAA1093 protein, | 22 | | | | | | | | | | | |
| | | | | | | | | | | | | | | KIAA1093 | | | | | | | | | | | | | | |
| 41522_at | MFNG | Z93096 | 6 | Pass | 10 33 | 3 72 | 13 | Pass | TRUE | FALSE | FALSE | 5 23 | 1.98 | manic fringe (Drosophila) | 22q12 | | | | | | | | | | | | | |
| | | | | | | | | | | | | | | homolog, MFNG | | | | | | | | | | | | | | |
| 36637_at | ANXA11 | L19605 | 6 | Pass | 25 83 | 7 41 | 13 | Pass | TRUE | FALSE | FALSE | 13.08 | 1.98 | american A11, ANXA11 | 10q22- | | | | | | | | | | | | | |
| | | | | | | | | | | | | | | | q23 | | | | | | | | | | | | | |
| 553_g_at | ARHGAP1 | U02570 | 6 | Pass | 43 00 | 15 65 | 13 | Pass | TRUE | FALSE | FALSE | 21 77 | 1.98 | Rho GTPase activating | | | | | | | | | | | | | | |
| | | | | | | | | | | | | | | protein 1, ARHGAP1 | | | | | | | | | | | | | | |
| 32201_at | SSNA1 | Z96932 | 4 | Pass | 19 50 | 7 19 | 8 | Pass | TRUE | FALSE | FALSE | 9.88 | 1.97 | Sjogren's syndrome | | | | | | | | | | | | | | |
| | | | | | | | | | | | | | | nuclear autoantigen 1, | | | | | | | | | | | | | | |
| 39180_at | FUS | S62140 | 6 | Pass | 47 83 | 13 29 | 13 | Pass | TRUE | FALSE | FALSE | 24 23 | 1.97 | fusion, derived from | 16p11 2 | | | | | | | | | | | | | |
| | | | | | | | | | | | | | | t(12,16) malignant | | | | | | | | | | | | | | |
| | | | | | | | | | | | | | | liposarcoma; FUS | | | | | | | | | | | | | | |
| 1062_g_at | IL10RA | U00672 | 6 | Pass | 47 67 | 15 71 | 13 | Pass | TRUE | FALSE | FALSE | 24 15 | 1.97 | interleukin 10 receptor, | 11q23 | | | | | | | | | | | | | |
| | | | | | | | | | | | | | | alpha, IL10RA | | | | | | | | | | | | | | |
| 41375_at | UNK_AJ2 | A3245416 | 6 | Pass | 15 17 | 5 88 | 13 | Pass | TRUE | FALSE | FALSE | 7 69 | 1.97 | Homo sapiens LST1 | | | | | | | | | | | | | | |
| | | | | | | | | | | | | | | mRNA | | | | | | | | | | | | | | |
| 36199_at | DAP | X76105 | 6 | Pass | 21 67 | 3 88 | 12 | Pass | TRUE | FALSE | FALSE | 11 00 | 1.97 | death-associated protein, | 5p15 2 | | | | | | | | | | | | | |
| | | | | | | | | | | | | | | DAP | | | | | | | | | | | | | | |
| 37345 at | CALU | AF013759 | 5 | Pass | 6 40 | 1.14 | 12 | Pass | TRUE | FALSE | FALSE | 3 25 | 1.97 | calumenin, CALU | 7q32 | | | | | | | | | | | | | |
| 38637_s_at | CLTA | M20471 | 6 | Pass | 43.17 | 19 93 | 13 | Pass | TRUE | FALSE | FALSE | 21.92 | 1.97 | clathium, light polypeptide | 12q23- | | | | | | | | | | | | | |
| | | | | | | | | | | | | | | (Lca), CLTA | q24 | | | | | | | | | | | | | |
| 32490_at | CEACAM | AC005955 | 5 | Pass | 9 00 | 2.92 | 7 | Pass | TRUE | FALSE | FALSE | 4 57 | 1.97 carcinoenbryone antigen- | 19q13 2 | | | | | | | | | | | | | | |
| | | | | | | | | | | | | | | related cell adhesion | | | | | | | | | | | | | | |
| | | | | | | | | | | | | | | molecule 4, CEACAM4 | | | | | | | | | | | | | | |
| 37739_at | SSRP1 | M86737 | 6 | Pass | 16.50 | 4.55 | 13 | Pass | TRUE | FALSE | FALSE | 8 38 | 1.97 | structure specific | 11q12 | | | | | | | | | | | | | |
| | | | | | | | | | | | | | | recognition protein 1, | | | | | | | | | | | | | | |
| | | | | | | | | | | | | | | SSRP1 | | | | | | | | | | | | | | |
| 32806_at | BZRP | M36035 | 6 | Pass | 248.17 | 116 68 | 13 | Pass | TRUE | FALSE | FALSE | 126.15 | 1.97 | benzodiazaprine receptor | 22q13 31 | | | | | | | | | | | | | |
| | | | | | | | | | | | | | | (peripheral), BZRP | | | | | | | | | | | | | | |
| 31891 at | CH13L2 | U5815 | 6 | Pass | 6 33 | 1 51 | 9 | Pass | TRUE | FALSE | FALSE | 3 22 | 1.97 | chutinase 3-like 2, CH13L2 | 1p13 3 | | | | | | | | | | | | | |
| 31801 at | UNK AI80 | AI808712 | 6 | Pass | 13.00 | 6 26 | 13 | Pass | TRUE | FALSE | FALSE | 6 62 | 1.97 | | | | | | | | | | | | | | | |
| 34787_at | NRD1 | X93209 | 6 | Pass | 32.17 | 9.64 | 13 | Pass | TRUE | FALSE | FALSE | 16.38 | 1.96 | nardilysin (N-arginine | 1p32 2- | | | | | | | | | | | | | |
| | | | | | | | | | | | | | | dibasic convertase), NRD1 | p32 1 | | | | | | | | | | | | | |
| 36666_at | P4HB | M22806 | 6 | Pass | 77.17 | 21 94 | 13 | Pass | TRUE | FALSE | FALSE | 39.31 | 1.96 | procollagen-proline, 2- | 17q25 | | | | | | | | | | | | | |
| | | | | | | | | | | | | | | oxoglutarate 4- | | | | | | | | | | | | | | |
| | | | | | | | | | | | | | | dioxygenase (proline 4- | | | | | | | | | | | | | | |
| | | | | | | | | | | | | | | hydroxylase), beta | | | | | | | | | | | | | | |
| | | | | | | | | | | | | | | polypeptide (protein | | | | | | | | | | | | | | |
| | | | | | | | | | | | | | | disulfide isomerase, | | | | | | | | | | | | | | |
| | | | | | | | | | | | | | | thryoid hormone binding | | | | | | | | | | | | | | |
| 39738 at | APOL | Z82215 | 6 | Pass | 152 17 | 26 57 | 13 | Pass | TRUE | FALSE | FALSE | 77 62 | 1.96 | | | | | | | | | | | | | | | |
| 40842_at | SNRPA | M60784 | 6 | Pass | 36 17 | 8 80 | 13 | Pass | TRUE | FALSE | FALSE | 18 46 | 1.96 | smal nuclear | 19q13 1 | | | | | | | | | | | | | |
| | | | | | | | | | | | | | | ribonucleoprotein | | | | | | | | | | | | | | |
| | | | | | | | | | | | | | | polypeptide A, SNRPA | | | | | | | | | | | | | | |
| 39551_at | UNK_N98 | N98667 | 6 | Pass | 25 00 | 9.57 | 13 | Pass | TRUE | FALSE | FALSE | 12.77 | 1.96 | hypothetical protein; | | | | | | | | | | | | | | |
| | | | | | | | | | | | | | | LOC51317 | | | | | | | | | | | | | | |
| 35017_f_at | UNK_M80 | M80469 | 6 | Pass | 129 67 | 39.72 | 13 | Pass | TRUE | FALSE | FALSE | 66 23 | 1.96 | Cluster Incl M80169 | | | | | | | | | | | | | | |
| | | | | | | | | | | | | | | Human MHC clas 1 HLA- | | | | | | | | | | | | | | |
| | | | | | | | | | | | | | | J gene, exons 1-8 and | | | | | | | | | | | | | | |
| | | | | | | | | | | | | | | complete eds | | | | | | | | | | | | | | |
| 894_g_at | E2-EPF | M91670 | 6 | Pass | 13.50 | 4.85 | 10 | Pass | TRUE | FALSE | FALSE | 6 90 | 1.96 | ubiquitin carrier protein, | 17 | | | | | | | | | | | | | |
| | | | | | | | | | | | | | | E2-EPF | | | | | | | | | | | | | | |
| 590 at | ICAM2 | M32334 | 6 | Pass | 26.33 | 4.72 | 13 | Pass | TRUE | FALSE | FALSE | 13.46 | 1.96 | intercellular adhesion | 17q23- | | | | | | | | | | | | | |
| | | | | | | | | | | | | | | molecule 2, ICAM2 | q25 | | | | | | | | | | | | | |
| 34346_at | PRKAG1 | U42412 | 6 | Pass | 16.83 | 6 01 | 13 | Pass | TRUE | FALSE | FALSE | 8 62 | 1.95 | protein kinase, AMP- | 12q12- | | | | | | | | | | | | | |
| | | | | | | | | | | | | | | activated, gamma 1 non- | q14 | | | | | | | | | | | | | |
| | | | | | | | | | | | | | | catalytic subunit, | | | | | | | | | | | | | | |
| 39500 s at | UNK AL0 | AL049299 | 5 | Pass | 8 40 | 2.70 | 10 | Pass | FALSE | FALSE | 4 30 | 1.95 | | | | | | | | | | | | | | | | |
| 34865_at | NDUFS6 | AI360249 | 5 | Pass | 9 60 | 4.34 | 12 | Pass | TRUE | FALSE | FALSE | 4 92 | 1.95 | NADH dehydrogenase | | | | | | | | | | | | | | |
| | | | | | | | | | | | | | | (ubiquinone) Fe—S protein | | | | | | | | | | | | | | |
| | | | | | | | | | | | | | | 6 (13kD) (NADH- | | | | | | | | | | | | | | |
| | | | | | | | | | | | | | | coenzyme O reductase), | | | | | | | | | | | | | | |
| 37344_at | HLA-DMA | X62744 | 6 | Pass | 51.50 | 18 12 | 13 | Pass | TRUE | FALSE | FALSE | 26 38 | 1.95 | major histocompatibility | 6q21 3 | | | | | | | | | | | | | |
| | | | | | | | | | | | | | | complex, class II DM | | | | | | | | | | | | | | |
| | | | | | | | | | | | | | | alpha, IILA-DMA | | | | | | | | | | | | | | |
| 34692_r_{—l at} | ARPC4 | AF006087 | 6 | Pass | 22 67 | 7.84 | 13 | Pass | TRUE | FALSE | FALSE | 11 62 | 1.95 actin related protein 2/3 | | | | | | | | | | | | | | | |
| | | | | | | | | | | | | | | complex, subunit 4(20 | | | | | | | | | | | | | | |
| | | | | | | | | | | | | | | kD), ARPC4 | | | | | | | | | | | | | | |
| 41776_at | ATOX1 | U70660 | 6 | Pass | 13.33 | 4 50 | 12 | Pass | TRUE | FALSE | FALSE | 6 83 | 1.95 | ATX1 (antioxidant protein | 5q32 | | | | | | | | | | | | | |
| | | | | | | | | | | | | | | 1, yeast) homolog1, | | | | | | | | | | | | | | |
| | | | | | | | | | | | | | | ATOX1 | | | | | | | | | | | | | | |
| 34885 at | SYNGR2 | AJ002308 | 6 | Pass | 75 17 | 17.66 | 13 | Pass | TRUE | FALSE | FALSE | 38.54 | 1.95 | synaptogyin 2, SYNGR2 | 17qter | | | | | | | | | | | | | |
| 893_at | E2-EPF | M91670 | 5 | Pass | 7.80 | 1 79 | 9 | Pass | TRUE | FALSE | FALSE | 4.00 | 1.95 | ubiquitin carrier protein, | 17 | | | | | | | | | | | | | |
| | | | | | | | | | | | | | | E2-EPF | | | | | | | | | | | | | | |
| 33956 | MD-2 | AB018549 | 6 | Pass | 13 50 | 7 89 | 13 | Pass | TRUE | FALSE | FALSE | 6 92 | 1.95 | MD-2 protein, MD-2 | 8 | | | | | | | | | | | | | |
| 38725_s_at | DPM2 | N36295 | 8 | Pass | 10 50 | 3 56 | 13 | Pass | TRUE | FALSE | FALSE | 5 38 | 1.95 | clohelyl-phosphatic | | | | | | | | | | | | | | |
| | | | | | | | | | | | | | | mannosyltransferase | | | | | | | | | | | | | | |
| | | | | | | | | | | | | | | polypeptide 2, regulatory | | | | | | | | | | | | | | |
| | | | | | | | | | | | | | | subunit, DPM2 | | | | | | | | | | | | | | |
| 40928_at | DKFZP564 | W264096 | Pass | 10.50 | 8.41 | 13 | Pass | TRUE | FALSE | FALSE | 5 38 | 1.95 | DKFZP564A122 protein, | | | | | | | | | | | | | | | |
| | | | | | | | | | | | | | | DKFZP564A122 | | | | | | | | | | | | | | |
| 39811 at | UNK AA4 | AA402538 | 6 | Pass | 26 83 | 8.45 | 13 | Pass | TRUE | FALSE | FALSE | 13 77 | 1.95 | | | | | | | | | | | | | | | |
| 36103_at | SCYA3 | D90144 | 6 | Pass | 6.33 | 2.80 | 12 | Pass | TRUE | FALSE | FALSE | 3 25 | 1.95 | small inducible cytokine | 17q11- | | | | | | | | | | | | | |
| | | | | | | | | | | | | | | A3 (homologous to mouse | q21 | | | | | | | | | | | | | |
| | | | | | | | | | | | | | | Mip-1a), SCYA3 | | | | | | | | | | | | | | |
| 40593_at | PTB | X66975 | 6 | Pass | 64 00 | 23 03 | 13 | Pass | TRUE | FALSE | FALSE | 32 85 | 1.95 | polypyrintidine tract | 14q23- | | | | | | | | | | | | | |
| | | | | | | | | | | | | | | binding protein | q24.1 | | | | | | | | | | | | | |
| | | | | | | | | | | | | | | (heterogeneous nuclear | | | | | | | | | | | | | | |
| | | | | | | | | | | | | | | ribonucleopotein 1), PTB | | | | | | | | | | | | | | |
| 1532_g_at | UNK_U50 | U50535 | 6 | Pass | 16 33 | 3.44 | 13 | Pass | TRUE | FALSE | FALSE | 8.38 | 1.95 | Human BRCA2 region, | | | | | | | | | | | | | | |
| | | | | | | | | | | | | | | mRNA sequence CG006 | | | | | | | | | | | | | | |
| 32264_at | UNK_L23 | L23134 | 5 | Pass | 29.60 | 14 67 | 10 | Pass | TRUE | FALSE | FALSE | 15 20 | 1.95 | granzyme M (lymphocyte | 19q13 3 | | | | | | | | | | | | | |
| | | | | | | | | | | | | | | | met-ase 1), GZMM | | | | | | | | | | | | | |
| 1097_s_at | CCR7 | L31584 | 6 | Pass | 44.33 | 27.45 | 13 | Pass | TRUE | FALSE | FALSE | 22 77 | 1.95 | chemokine (C—C mofit) | 17q12- | | | | | | | | | | | | | |
| | | | | | | | | | | | | | | receptor 7, CCR7 | q21 2 | | | | | | | | | | | | | |
| 35753_at | PRP8 | AB007510 | 6 | Pass | 56.00 | 9 06 | 13 | Pass | TRUE | FALSE | FALSE | 28.77 | 1.95 | US snRNP-specific protein | l7p13 3 | | | | | | | | | | | | | |
| | | | | | | | | | | | | | | (220 kD), ortholog of S | | | | | | | | | | | | | | |
| | | | | | | | | | | | | | | cerevisiae Prp8p, PRP8 | | | | | | | | | | | | | | |
| 36129_at | UNK_AB0 | AB007857 | 6 | Pass | 25.00 | 4.69 | 13 | Pass | TRUE | FALSE | FALSE | 12 85 | 1.95 | KIAA0397 gene product, | | | | | | | | | | | | | | |
| | | | | | | | | | | | | | | KIAA0397 | | | | | | | | | | | | | | |
| 33367_s_at | L0C51582 | D88674 | 6 | Pass | 4.33 | 2 34 | 10 | Pass | TRUE | FALSE | FALSE | 8 50 | −1.96 | antizyme | 10p13 2,8 | | | | | | | | | | | | | |
| | | | | | | | | | | | | | | inhibitor protease, serine, | | | | | | | | | | | | | | |
| | | | | | | | | | | | | | | 15, LOC51582,PRSS15 | | | | | | | | | | | | | | |
| AFFX-M2783 | 28SRNA5 | M27830 | 6 | Pass | 9.17 | 6.01 | 13 | Pass | TRUE | FALSE | FALSE | 18 46 | −2.61 | #N/A | #N/A | | | | | | | | | | | | | |
| 34642_at | YWHAZ | U28964 | 6 | Pass | 15.17 | 10.83 | 13 | Pass | TRUE | FALSE | FALSE | 30.62 | −2.02 | tyrosine 3- | 2p25.2- | | | | | | | | | | | | | |
| | | | | | | | | | | | | | | monooxygenase/tryptopha | p25.1 | | | | | | | | | | | | | |
| | | | | | | | | | | | | | | n 5-monooxygenase | | | | | | | | | | | | | | |
| | | | | | | | | | | | | | | activation protein,zeta | | | | | | | | | | | | | | |
| | | | | | | | | | | | | | | polypeptide, YWHAZ | | | | | | | | | | | | | | |

| | | | | | | | | | | | Absent in | | | | | |
|---|---|---|---|---|---|---|---|---|---|---|---|---|---|---|---|---|
| | | | | | | | | | Present | Present | RA, | | Fold | | | |
| | | | | | | Std | sum of | | in RA | in RA, | Present | Avg | Change | | | |
| | | | sum of | 4 of 6 | Avg | Dev | abs | 7 of 13 | and | Absent in | in | Freq | RA/ | | Chromo- | |
| qualifier | name | | abs dec | present | Freq | RA | dec | present | Normal | Normal | Normal | (Normal) | Normal | Name | some | |
| 1642_at | MTA1 | U35113 | 4 | Pass | 4 75 | 1 26 | 0 | Fail | FALSE | TRUE | FALSE | #DIV/0¹ | #DIV/ | metastasis associated 1, | | |
| | | | | | | | | | | | | | 01 | MTA1 | | |
| 37963 at | ARSA | X52151 | 4 | Pass | 3 50 | 1 73 | 0 | Fail | FALSE | TRUE | FALSE | #DIV/0¹ | #DIV/ | arylsulfatase A, ARSA | 22q13 33 | |
| | | | | | | | | | | | | | 01 | | | |
| 38350 f at | TUBA2 | AF005392 | 4 | Pass | 10 25 | 1 26 | 0 | Fail | FALSE | TRUE | FALSE | #DIV/0¹ | #DIV/ | tubulin, alpha 2, TUBA2 | 13q11 | |
| | | | | | | | | | | | | | 01 | | | |
| 40800_at | UNK_AI59 | AI590869 | 4 | Pass | 6 75 | 1 71 | 0 | Fail | FALSE | TRUE | FALSE | #DIV/0¹ | #DIV/ | hypothetical protein | 16 | |
| | | | | | | | | | | | | | 01 | similar to mouse HN1 | | |
| | | | | | | | | | | | | | | (Hematological and | | |
| | | | | | | | | | | | | | | Neurological expressed | | |
| | | | | | | | | | | | | | | sequence 1), HN1L | | |
| 38021_at | PLEC1 | U53204 | 6 | Pass | 22 17 | 10 91 | 0 | Fail | FALSE | TRUE | FALSE | #DIV/0¹ | #DIV/ | plectin 1, intermediate | 8q24 | |
| | | | | | | | | | | | | | 01 | filament binding protein, | | |
| | | | | | | | | | | | | | | 500kD; PLEC1 | | |
| 1257 s at | QSCN6 | L42379 | 4 | Pass | 19.00 | 18 57 | 1 | Fail | FALSE | TRUE | FALSE | 3 00 | 6.33 | quesin Q6, QSCN6 | 1qn24 | |
| 39128_r_at | PPP2R4 | X73478 | 4 | Pass | 7.25 | 5.68 | 2 | Fail | FALSE | TRUE | FALSE | 1 50 | 4.83 | protein phosphalase 2A, | 9q34 | |
| | | | | | | | | | | | | | | regulatory subunit B′(PR | | |
| | | | | | | | | | | | | | | 53): PPP2R4 | | |
| 545_g_{—l at} | NFKB2 | 576638 | 4 | Pass | 4.50 | 1 29 | 1 | Fail | FALSE | TRUE | FALSE | 1 00 | 4.50 | nuclear factor of kappa | 10q24 | |
| | | | | | | | | | | | | | | light polypeptide gene | | |
| | | | | | | | | | | | | | | enhancer in B-cells 2 | | |
| | | | | | | | | | | | | | | (p49/p100): NFKB2 | | |
| 37769_at | EDG4 | AF011466 | 6 | Pass | 20 50 | 4 32 | 1 | Fail | FALSE | TRUE | FALSE | 5 00 | 4.10 | endothelial differentiation, | 19p12 | |
| | | | | | | | | | | | | | | lysophosphatidic acid G- | | |
| | | | | | | | | | | | | | | protein-coupled receptor, | | |
| | | | | | | | | | | | | | | 4, EDG4 | | |
| 2049_s_at | JUNB | M29039 | 5 | Pass | 10 80 | 7 19 | 6 | Fail | FALSE | TRUE | FALSE | 2.67 | 4.05 | jun B proto-oncogene, | 19p13.2 | |
| | | | | | | | | | | | | | | JUNB | | |
| 39145_at | MYRL2 | J02854 | 6 | Pass | 19 33 | 9.24 | 1 | Fail | FALSE | TRUE | FALSE | 5.00 | 3.87 | myosin regulatory light | | |
| | | | | | | | | | | | | | | chain 2, smooth muscle | | |
| | | | | | | | | | | | | | | isoform, MYRL2 | | |
| 32151_at | RANGAP1 | X82260 | 4 | Pass | 12 25 | 2 63 | 2 | Fail | FALSE | TRUE | FALSE | 3 50 | 3.50 | Ran GTPase activating | 22q13 2- | |
| | | | | | | | | | | | | | | protein 1, RANGAP1 | q13 31 | |
| 34367_at | PHGDH | AF006043 | 6 | Pass | 5.83 | 3.43 | 3 | Fail | FALSE | TRUE | FALSE | 1 67 3.50 | 3-phosphoglycerate | 1p11 1- | | |
| | | | | | | | | | | | | | | dehydrogenase, PHGDH | 13 1 | |
| 31596_f_at | UNK_L02 | L02326 | 6 | Pass | 46.50 17 20 | 4 | Fail | FALSE | TRUE | FALSE | 13.50 | 3.44 | immunoglobulin lambda- | 22q11 23 | | |
| | | | | | | | | | | | | | | like polypeptide 2, IGLL2 | | |
| 34105 f at | UNK AI14 | AI147237 | 4 | Pass | 53 75 | 31.42 | 6 | Fail | FALSE | TRUE | FALSE | 15 67 | 3.43 | | | |
| 1665_s_at | ECQF1 | M63193 | 4 | Pass | 77.75 | 68.10 | 3 | Fail | FALSE | TRUE | FALSE | 23 33 | 3.33 | endothelial cell growth | 22q13 33 | |
| | | | | | | | | | | | | | | factor 1 (platelet-derived), | | |
| | | | | | | | | | | | | | | ECCF1 | | |
| 1855_at | FGF3 | X14445 | 4 | Pass | 6 50 | 2.65 | 3 | Fail | FALSE | TRUE | FALSE | 2.00 | 3.25 | fibroblast growth factor 3 | 11q13 | |
| | | | | | | | | | | | | | | (murine mammary tumor | | |
| | | | | | | | | | | | | | | virus integration site (v-mut- | | |
| | | | | | | | | | | | | | | 2) oncogene homolog), | | |
| | | | | | | | | | | | | | | FGF3 | | |
| 36151_at | HU-K4 | U60644 | 5 | Pass | 13 00 | 4 47 | 5 | Fail | FALSE | TRUE | FALSE | 4.00 | 3.25 | similar to vaccinia vaus | | |
| | | | | | | | | | | | | | | HundU1 K4L ORF, HU-K4 | | |
| 32133_at | PIPSK1C | AB011161 | 6 | Pass | 14 67 | 5 99 | 5 | Fail | FALSE | TRUE | FALSE | 4.60 | 3.19 | phosphatidylinositol-4- | 19 | kinase |
| | | | | | | | | | | | | | | phosphate 5-kinase, type 1, | | |
| | | | | | | | | | | | | | | gamma, PIP5K1C | | |
| | | | | | | | | | Present | Present | Absent in | Avg | Fold | | | |
| | | | sum of | | Avg | Std | sum of | | in RA | in RA, | Freq | Change | | | | |
| Affy | Affy | | Present | 4 of 6 | Freq | Dev | abs | 7 of 13 | and | Absent in | (Nor- | RA/ | | Chromo- | Kinase or | |
| Qualifier | Name | accession | Calls | present | RA | RA | dec | present | Normal | Normal | mal) | Normal | Name | some | Phosphatase | |
| 41659_at | SUPT6H | U46691 | 4 | Pass | 4.50 | 2 38 | 2 | Fail | FALSE | TRUE | FALSE | 1.50 | 3.80 | suppresor of Ty | 17q11.2 | |
| | | | | | | | | | | | | | | (S cerevisiae) 6 homolog; | | |
| | | | | | | | | | | | | | | SUPT611 | | |
| 36158_at | DCTN1 | AF086947 | 5 | Pass | 7.80 | 1.92 | 5 | Fail | FALSE | TRUE | FALSE | 2 60 | 3.00 | dyactin 1 (p150, Glued | 2p13 | |
| | | | | | | | | | | | | | | (Drosophilia) homolog), | | |
| | | | | | | | | | | | | | | DCTN1 | | |
| 35936_g_at | CPT1B | Y08683 | 5 | Pass | 8 00 | 2 35 | 4 | Fail | FALSE | TRUE | FALSE | 2 75 | 2.91 | carnitine | 22q13 33 | |
| | | | | | | | | | | | | | | palmitoyltransferase 1, | | |
| | | | | | | | | | | | | | | muscle, CPT1B | | |
| 41458_at | KIAA0467 | AB007936 | 4 | Pass | 14 50 | 5 20 | 1 | Fail | FALSE | TRUE | FALSE | 5 00 | 2.90 | KIAA0467 protein, 1 | | |
| | | | | | | | | | | | | | | KIAA0467 | | |
| 33719 at | UNK AF0 | AF010242 | 4 | Pass | 8 50 | 1 73 | 2 | Fail | FALSE | TRUE | FALSE | 3 00 | 2.83 | | | |
| 39306_at | PRSS16 | AF052514 | 4 | Pass | 8.50 | 3.51 | 2 | Fail | FALSE | TRUE | FALSE | 3 00 | 2.83 | protease serine, 16 | 6p21 | |
| | | | | | | | | | | | | | | (thymus), PRSS16 | | |
| 37365_at | HS11 | X63368 | 4 | Pass | 7.75 | 2.22 | 4 | Fail | FALSE | TRUE | FALSE | 2 75 | 2.82 | heat shock protein, | 2q32-q34 | |
| | | | | | | | | | | | | | | neuronal DNA1-like 1, | | |
| | | | | | | | | | | | | | | HS11 | | |
| 40951 at | UNK AL0 | AL049250 | 5 | Pass | 6.40 | 5 50 | 3 | Fail | FALSE | TRUE | FALSE | 2 33 | 2.74 | | | |
| 41351 at | UNK AA8 | AA885106 | 4 | Pass | 26 25 | 7.85 | 6 | Fail | FALSE | TRUE | FASLE | 9.83 | 2.67 | | | |
| 35228_at | CTIB | Y08682 | 5 | Pass | 9.60 | 2.88 | 5 | Fail | FALSE | TRUE | FALSE | 3 60 | 2.67 | carritine | 22q13 33 | |
| | | | | | | | | | | | | | | polymiotoyltransferase 1, | | |
| | | | | | | | | | | | | | | muscle, CPTIB | | |
| 40165 at | UNK AB0 | AB015345 | 6 | Pass | 5 33 | 1.63 | 5 | Fail | FALSE | TRUE | FALSE | 2 00 | 2.67 | | | |
| 40159_r_at | NCF1 | M55067 | 6 | Pass | 70 83 | 52 24 | 5 | Fail | FALSE | TRUE | FALSE | 26 80 | 2.64 | neutrophil cytosolic factor | 3q11 23 | |
| | | | | | | | | | | | | | | 1 (47kD, chrome | | |
| | | | | | | | | | | | | | | granulomatous disease, | | |
| | | | | | | | | | | | | | | autosomal 1). NCF1 | | |
| 39837 s at | UNK AC0 | AC004877 | 4 | Pass | 14 50 | 8 74 | 4 | Fail | FALSE | TRUE | FALSE | 5 50 | 2.64 | | | |
| 23374_at | C2 | L09708 4 | Pass | 5 25 | 0 96 | 1 | Fail | FALSE | TRUE | FALSE | 2 00 | 2.63 | complement component 2, | 6p21 3 | | |
| | | | | | | | | | | | | | | C2 | | |
| 2009_at | PTK2B | U33284 | 4 | Pass | 5 25 | 1 50 | 2 | Fail | FALSE | TRUE | FALSE | 2 00 | 2.63 | protein tyrosine kinase | 8p21 1 | Kinase |
| | | | | | | | | | | | | | | beta, PTK2B | | |
| 41083 at | UNK AC0 | AC006276 | 4 | Pass | 5 25 | 1 71 | 3 | Fail | FALSE | TRUE | FALSE | 2 00 | 2.63 | | | |
| 37656_at | SCAP | D83782 | 5 | Pass | 11 00 | 3.94 | 4 | Fail | FALSE | TRUE | FALSE | 4 25 | 2.59 | SREBP CLEAVAGE- | 3 | |
| | | | | | | | | | | | | | | ACTIVATING PROTEIN, | | |
| | | | | | | | | | | | | | | SCAP | | |
| 100_g_at | RABGGTA | Y08200 5 | Pass | 9 40 | 4.16 | 3 | Fail | FALSE | TRUE | FALSE | 3 67 | 2.56 | Rab | 14q11 2 | | |
| | | | | | | | | | | | | | | geranylgetanyltransferase, | | |
| | | | | | | | | | | | | | | alpha subunit RABGGTA | | |
| 41648_at | CRAT | X78706 | 4 | Pass | 20 50 | 3 11 | 4 | Fail | FALSE | TRUE | FALSE | 8 00 | 2.56 | carrintine acetyltransferase, | 9q34 1 | |
| | | | | | | | | | | | | | | CRAT | | |
| 37977_at | PMS2L11 | AI138834 | 4 | Pass | 7 25 | 3 30 | 6 | Fail | FALSE | TRUE | FALSE | 2 83 | 2.56 | postnucrotic segregation | 7q | |
| | | | | | | | | | | | | | | increased 2-like 11, | | |
| | | | | | | | | | | | | | | PMS2L1 | | |
| 37401_at | TAF2A | D90359 | 4 | Pass | 15 75 | 6 99 | 6 | Fail | FALSE | TRUE | FALSE | 6 17 | 2.55 | TATA box binding protein | Xq13 1 | |
| | | | | | | | | | | | | | | (TBP)-associated factor, | | |
| | | | | | | | | | | | | | | RNA polymerase II, A, | | |
| | | | | | | | | | | | | | | 250kD TAF2A | | |
| 31410 at | TAC1 | AF023614 | 4 | Pass | 7 00 | 4 08 | 4 | Fail | FALSE | TRUE | FALSE | 2.75 | 2.55 | transmembrane activator | | |
| | | | | | | | | | | | | | | and CAML interactor, | | |
| | | | | | | | | | | | | | | TAC1 | | |
| 37360_at | LY6E | U66711 | 5 | Pass | 50 80 | 41 61 | 2 | Fail | FALSE | TRUE | FALSE | 20 00 | 2.54 | lymphocyte antigen 6 | 8q24 3 | |
| | | | | | | | | | | | | | | complex, locus E: LY6E | | |
| 35434_at | MEF2D | L16794 | 6 | Pass | 6 33 | 1.75 | 6 | Fail | FALSE | TRUE | FALSE | 2 50 | 2.53 | MADS box transcription | 1q12-q23 | |
| | | | | | | | | | | | | | | enhancer factor 2, | | |
| | | | | | | | | | | | | | | polypeptide D (myocte | | |
| | | | | | | | | | | | | | | enhancer factor 2D), | | |
| | | | | | | | | | | | | | | MEF2D | | |
| 38971_r_at | NAF1 | AJ011896 | 4 | Pass | 53 50 | 25 51 | 5 | Fail | FALSE | TRUE | FALSE | 21 20 | 2.52 | Nef-associated factor 1, | 5q32- | |
| | | | | | | | | | | | | | | NAF1 | q33 1 | |
| 36958 at | ZYX | X95735 | 6 | Pass | 42 83 | 22 68 | 4 | Fail | FALSE | TRUE | FALSE | 17.00 | 2.52 | zyxin, ZYX | 7q32 | |
| 34432_at | SH2D2A | AF051325 | 5 | Pass | 4 60 | 3 71 | 6 | Fail | FALSE | TRUE | FALSE | 1 83 | 2.51 | SH2 domain protein 2A, | 1q21 | |
| | | | | | | | | | | | | | | SH2D2A | | |
| 35194_at | GPX2 | X53463 | 4 | Pass | 5.00 | 1.63 | 1 | Fail | FALSE | TRUE | FALSE | 2.00 | 2.50 | glutathione peroxidase 2 | 14q24.1 | |
| | | | | | | | | | | | | | | (gastrointestinal); GPX2 | | |
| 38276_at | NFKB1E | U91616 | 4 | Pass | 5 00 | 0 82 | 1 | Fail | FALSE | TRUE | FALSE | 2 00 | 2.50 | unclear factor of kappa | | |
| | | | | | | | | | | | | | | high polypeptide gene | | |
| | | | | | | | | | | | | | | enhancer in B-cells | | |
| | | | | | | | | | | | | | | inhibitor,, cpsilon | | |
| 40191_s_at | UNK_AI76 | AI761647 | 5 | Pass | 5 00 | 2 00 | 1 | Fail | FALSE | TRUE | FALSE | 2 00 | 2.50 | KIAA0582 protein, | 2 | |
| | | | | | | | | | | | | | | KIAA0582 | | |
| 35079 at | NB-3 | AB003592 | 4 | Pass | 5 00 | 0 82 | 4 | Fail | FALSE | TRUE | FALSE | 2 00 | 2.50 | contactin 6, CNTN6 | 3p26-p25 | |
| 36598_s_{—l at} | INPPL1 | L36818 | 6 | Pass | 9 17 | 4 07 | 6 | Fail | FALSE | TRUE | FALSE | 3.67 | 2.50 | mositol polyphosphate | 11q23 | Phosphatase |
| | | | | | | | | | | | | | | phosphatase-like 1, | | |
| | | | | | | | | | | | | | | INPPL1 | | |
| 35601 at | UNK L000 | L00022 | 6 | Pass | 16 83 | 9 47 | 6 | Fail | FALSE | TRUE | FALSE | 6 83 | 2.46 | | | |
| 37719_at | MLF2 | AF070539 | 4 | Pass | 11 00 | 3 56 | 2 | Fail | FALSE | TRUE | FALSE | 4.50 | 2.44 | myeloid leukemia factor 2, | 12p13 | |
| | | | | | | | | | | | | | | MLT2 | | |
| 38464 at | GCS1 | X87237 | 5 | Pass | 12 20 | 3 42 | 4 | Fail | FALSE | TRUE | FALSE | 5 00 | 2.44 | glucosidase 1, GCS1 | 2p13-p12 | |
| 40639_at | SCO2 | AL021683 | 4 | Pass | 14 50 | 10 63 | 1 | Fail | FALSE | TRUE | FALSE | 6.00 | 2.42 | SCO (cytochrome oxidase | 22q13 33 | |
| | | | | | | | | | | | | | | deficient, yeast) homolog | | |
| | | | | | | | | | | | | | | 2, SCO2 | | |
| 41273 at | UNK AL0 | AL046940 | 4 | Pass | 6 25 | 0 50 | 5 | Fail | FALSE | TRUE | FALSE | 2 60 | 2.40 | | | |
| 35961 at | UNK AL0 | AL049390 | 5 | Pass | 4 80 | 1 10 | 4 | Fail | FALSE | TRUE | FALSE | 2 00 | 2.40 | | | |
| 38721_at | HBP1 | W72733 | 6 | Pass | 8 50 | 1 22 | 5 | Fail | FALSE | TRUE | FALSE | 3 60 | 2.36 | HMG-box containing | 7q31 1 | |
| | | | | | | | | | | | | | | protein1, HBP1 | | |
| 519_g_at | NR1H2 | U07132 | 6 | Pass | 25 17 | 7 57 | 3 | Fail | FALSE | TRUE | FALSE | 10 67 | 2.36 | nuclear receptor subfamily | 19q13 3- | |
| | | | | | | | | | | | | | | 1, group H, member 2, | 19q13 3 | |
| | | | | | | | | | | | | | | NR1H2 | | |
| 41127_at | SLC1A4 | L14595 | 5 | Pass | 6 20 | 2 28 | 6 | Fail | FALSE | TRUE | FALSE | 2 67 | 2.33 | solute carrier family 1 | 2p15-p13 | |
| | | | | | | | | | | | | | | (gtutamate/neutral amino | | |
| | | | | | | | | | | | | | | acid transporter), member | | |
| | | | | | | | | | | | | | | 4, SC1A-4 | | |
| 38340_at | KIAA0655 | AB014555 | 4 | Pass | 12 75 | 3.20 | 6 | Fail | FALSE | TRUE | FALSE | 5.50 | 2.32 | huntington interacting | 12q24 | |
| | | | | | | | | | | | | | | protein-1-related, | | |
| | | | | | | | | | | | | | | KIAA0655 | | |
| 32177_s_at | GAPL | AC004084 | 4 | Pass | 9 25 | 2.22 | 2 | Fail | FALSE | TRUE | FALSE | 4.00 | 2.31 | GTPase activating protein- | 7q22- | |
| | | | | | | | | | | | | | | like, GAPL | | |
| 736 f at | POM121L | D87002 | 4 | Pass | 10 00 | 2.58 | 3 | Fail | FALSE | TRUE | FALSE | 4.33 | 2.31 | | | |
| 1550_at | MAAT1 | U19796 | 4 | Pass | 8 00 | 2.45 | 2 | Fail | FALSE | TRUE | FALSE | 3 50 | 2.29 | melanoma-associated | | |
| | | | | | | | | | | | | | | antigen recognised by T | | |
| | | | | | | | | | | | | | | lymphocytes, MAAT1 | | |
| 37945_at | HBACH | U91316 | 4 | Pass | 6 25 | 1.71 | 4 | Fail | FALSE | TRUE | FALSE | 2.75 | 2.27 | cytosolic acyl coenzyme A | 1p36 31- | |
| | | | | | | | | | | | | | | thioester hydrolase, | p36 11 | |
| | | | | | | | | | | | | | | IIBACH | | |
| 31822_at | CUTL1 | L12579 | 5 | Pass | 9.40 | 3.29 | 6 | Fail | FALSE | TRUE | FALSE | 4.17 | 2.26 | cut (Drosophila)-like 1 | 7q22 | |
| | | | | | | | | | | | | | | (CCAAT displacement | | |
| | | | | | | | | | | | | | | protein), CUTL1 | | |
| 33105 at | UNK W28 | W28790 | 4 | Pass | 5 25 | 2 63 | 3 | Fail | FALSE | TRUE | FALSE | 2 33 | 2.25 | | | |
| 35149_at | TNFRSF5 | AI865431 | 4 | Pass | 6.75 | 3 59 | 6 | Fail | FALSE | TRUE | FALSE | 3 00 | 2.25 | tumor necrosis factor | 2oq12- | |
| | | | | | | | | | | | | | | receptor superfamily, | q13 2 | |
| | | | | | | | | | | | | | | member 5; TNFRSF5 | | |
| 40619_at | E2-EPF | M91670 | 4 | Pass | 8.50 | 1 00 | 6 | Fail | FALSE | TRUE | FALSE | 3 83 | 2.22 | ubiquitin carrier protein, | 17 | |
| | | | | | | | | | | | | | | E2-EPF | | |
| 570_at | RELB | M83221 | 6 | Pass | 8.00 | 3 35 | 6 | Fail | FALSE | TRUE | FALSE | 3 67 | 2.18 | v-tel avian | | |
| | | | | | | | | | | | | | | reticuloendotheliosis viral | | |
| | | | | | | | | | | | | | | oncogene homolog B | | |
| | | | | | | | | | | | | | | (nuclear factor of kappa | | |
| | | | | | | | | | | | | | | light polypeptide gene | | |
| | | | | | | | | | | | | | | enhancer in B-cells 3), | | |
| | | | | | | | | | | | | | | RELB | | |
| 1007_s_at | DDR1 | U48705 | 4 | Pass | 6 00 | 3 37 | 4 | Fail | FALSE | TRUE | FALSE | 2 75 | 2.18 | discordin domain receptor | 6p21.13 | Kinase |
| | | | | | | | | | | | | | | family, member 1, DDR1 | | |
| 36856 at | UNK W28 | W28743 | 5 | Pass | 13 80 | 6 72 | 6 | Fail | FALSE | TRUE | FALSE | 6 33 | 2.18 | | | |
| 37754_at | LGALS3B | L13210 | 4 | Pass | 3.25 | 1.50 | 4 | Fail | FALSE | TRUE | FALSE | 1.50 | 2.17 | lectin, galactoside-binding, | 17q25 | |
| | | | | | | | | | | | | | | soluble, 3 binding protein | | |
| | | | | | | | | | | | | | | (galactin 6 binding | | |
| | | | | | | | | | | | | | | protein), LGALS3BP | | |
| 33387_at | GAS7 | AB007854 | 4 | Pass | 6.00 | 1 83 | 5 | Fail | FALSE | TRUE | FALSE | 2 80 | 2.14 | growth anest-specific 7, | 17p | |
| | | | | | | | | | | | | | | GAS7 | | |
| 35960_at | IKBKB | AF031416 | 4 | Pass | 9.25 | 2 22 | 5 | Fail | FALSE | TRUE | FALSE | 4 33 | 2.13 | inhibitor of kappa light | 8p11 2 | Kinase |
| | | | | | | | | | | | | | | polypeptide gene enhancer | | |
| | | | | | | | | | | | | | | in B-cells, kinase beta, | | |
| | | | | | | | | | | | | | | IKBKB | | |
| 40359_at | C11ORF13 | M91083 | 5 | Pass | 19 20 | 4 09 | 4 | Fail | FALSE | TRUE | FALSE | 9 00 | 2.13 | chromosome 11 open | 11p15 5 | |
| | | | | | | | | | | | | | | reading frame 13, | | |
| | | | | | | | | | | | | | | C11ORF13 | | |
| 1397 at | MAP3K11 | L32976 | 4 | Pass | 4 25 | 1 50 | 3 | Fail | FALSE | TRUE | FALSE | 2.00 | 2.13 | mitogen-activated protein | 11q13 1- | Kinase |
| | | | | | | | | | | | | | | kinase kinase kinase 11, | q13.3 | |
| | | | | | | | | | | | | | | MAP3K11 | | |
| 33528_at | KIAA0125 | D50915 | 4 | Pass | 4 25 | 0 96 | 3 | Fail | FALSE | TRUE | FALSE | 2 00 | 2.13 | KIAA0125 gene product, | | |
| | | | | | | | | | | | | | | KIAA0125 | | |
| 38995_at | CLDN5 | AF000959 | 4 | Pass | 14.00 | 3 74 | 5 | Fail | FALSE | TRUE | FALSE | 6 60 | 2.12 | claudin 5 (transmembrane | 22q11 21 | |
| | | | | | | | | | | | | | | protein deleted in | | |
| | | | | | | | | | | | | | | velocardiofacial | | |
| | | | | | | | | | | | | | | syndrome), CLDNS | | |
| 39891 at | UNK A12 | A1246730 | 4 | Pass | 12 00 | 3 56 | 6 | Fail | FALSE | TRUE | FALSE | 5 67 | 2.12 | | | |
| 34669_at | TFE3 | X96717 | 5 | Pass | 6 00 | 1 22 | 6 | Fail | FALSE | TRUE | FALSE | 2 83 | 2.12 | transcription factor | Xp11 22 | Kinase |
| | | | | | | | | | | | | | | binding to IGHM enhancer | | |
| 40925 at | UNK AA5 | AA554945 | 4 | Pass | 9 50 | 2.65 | 6 | Fail | FALSE | TRUE | FALSE | 4 50 | 2.11 | | | |
| 1000_at | MAPK3 | X60188 | 6 | Pass | 7 33 | 3 08 | 6 | Fail | FALSE | TRUE | FALSE | 3 50 | 2.10 | mitogen-activated protein | 10p11 2 | Kinase |
| | | | | | | | | | | | | | | kinase 3, MAPK3 | | |
| 35763_at | UNK_AB0 | AB011112 | 6 | Pass | 8 83 | 5 64 | 4 | Fail | FALSE | TRUE | FALSE | 4 25 | 2.08 | KIAA0540 protein, | | |
| | | | | | | | | | | | | | | KIAA0540 | | |
| 41120_at | AMT | D14606 | 6 | Pass | 4 50 | 1 52 | 6 | Fail | FALSE | TRUE | FALSE | 2.17 | 2.08 | amminoethyltransferase | 3p21 2- | |
| | | | | | | | | | | | | | | (glycine cleavage system | p21 1 | |
| | | | | | | | | | | | | | | protein T), AMT | | |
| 703 at | UNK L235 | L23566 | 4 | Pass | 14 50 | 9 98 | 5 | Fail | FALSE | TRUE | FALSE | 7 00 | 2.07 | | | |
| 858_at | POR | S90469 | 4 | Pass | 7 75 | 3 30 | 4 | Fail | FALSE | TRUE | FALSE | 3 75 | 2.07 | P450 (cytochrome) | 7q11 2 | |
| | | | | | | | | | | | | | | oxidoreductase, POR | | |
| 1089 r at | UNK M64 | M64936 | 4 | Pass | 2 75 | 1 50 | 3 | Fail | FALSE | TRUE | FALSE | 1 33 | 2.06 | | | |
| 36004_at | IKBKG | AF074382 | 4 | Pass | 15.75 | 3 86 | 3 | Fail | FALSE | TRUE | FALSE | 7 67 | 2.05 | inhibitor of kappa light | Xq28 | |
| | | | | | | | | | | | | | | polypeptide gene enhancer | | |
| | | | | | | | | | | | | | | in B-cells, kinase gamma, | | |
| | | | | | | | | | | | | | | IKBKG | | |
| 34868_at | KIAA1089 | AB029012 | 5 | Pass | 4 60 | 1 14 | 4 | Fail | FALSE | TRUE | FALSE | 2 25 | 2.04 | KIAA1089 protein, | 1 | |
| | | | | | | | | | | | | | | KIAA1089 | | |
| 36184_at | PLOD | L06419 | 5 | Pass | 11 00 | 8 60 | 5 | Fail | FALSE | TRUE | FALSE | 5 40 | 2.04 | procollagen-lysine, 2- | 1p36 3- | |
| | | | | | | | | | | | | | | oxoglutate 5- | p36 2 | |
| | | | | | | | | | | | | | | dioxygenase (lysine | | |
| | | | | | | | | | | | | | | hydroxylase, Ehlers- | | |
| | | | | | | | | | | | | | | Danlos syndrome type VD | | |
| 39753_at | ITGA5 | X06256 | 4 | Pass | 9 50 | 4.65 | 3 | Fail | FALSE | TRUE | FALSE | 4.67 | 2.04 integrin alpha 5 | 12q11- | | |
| | | | | | | | | | | | | | | (fibronectin receptor, alpha | q13 | |
| | | | | | | | | | | | | | | polypeptide), ITGA5 | | |
| 36566_at | CTNS | AJ222967 | 4 | Pass | 4 00 | 1 41 | 1 | Fail | FALSE | TRUE | FALSE | 2.00 | 2.00 | cystinosis, nephropathic, | 17p13 | |
| | | | | | | | | | | | | | | CTNS | | |
| 32562_at | ENG | X72012 | 4 | Pass | 8 00 | 2 71 | 3 | Fail | FALSE | TRUE | FALSE | 4.00 | 2.00 | endogin (Oster-Rendu- | 9q33- | |
| | | | | | | | | | | | | | | Weber syndrome 1), ENG | q34 1 | |
| 495_at | IL15RA | U31628 | 4 | Pass | 4 00 | 0 82 | 3 | Fail | FALSE | TRUE | FALSE | 2.00 | 2.00 | interteukin 15 receptor, | 10p15- | |
| | | | | | | | | | | | | | | alpha, IL15RA | p14 | |
| 38020_at | KIAA0652 | AB014552 | 5 | Pass | 4 80 | 2 39 | 5 | Fail | FALSE | TRUE | FALSE | 2.40 | 2.00 | KIAA0652 gene product, | 11 | |
| | | | | | | | | | | | | | | KIAA0652 | | |
| 40730_at | CA4 | M83670 | 5 | Pass | 6 00 | 1.87 | 6 | Fail | FALSE | TRUE | FALSE | 3.00 | 2.00 | carbonic anhydrase IV; | 17q23 | |
| | | | | | | | | | | | | | | CA4 | | |
| 32802_at | TEB4 | AB011169 | 6 | Pass | 8.67 | 4 50 | 6 | Fail | FALSE | TRUE | FALSE | 4 33 | 2.00 | similar to S cerevisiae | 5p15 2 | |
| | | | | | | | | | | | | | | SSM4; TEB4 | | |
| 36307_at | KIAA0278 | D87468 | 5 | Pass | 12.80 | 4.55 | 4 | Fail | FALSE | TRUE | FALSE | 6.50 | 1.97 | activity-regulated | 8q24.3 | |
| | | | | | | | | | | | | | | cytoskeleton-associated | | |
| | | | | | | | | | | | | | | protein, ARC | | |
| 35230_at | CL24751 | AF070530 | 5 | Pass | 8.80 | 3.03 | 6 | Fail | FALSE | TRUE | FALSE | 4 50 | 1.96 | hypothetical protein, clone | 19 | |
| | | | | | | | | | | | | | | 24751, CL24751 | | |
| 35411_at | ATP-BL | AB018551 | 6 | Pass | 12.50 | 4.68 | 5 | Fail | FALSE | TRUE | FALSE | 6.40 | 1.95 | ATP synthase, subunit b- | 16q24 | |
| | | | | | | | | | | | | | | like, AIP-BL | | |
| 37098_at | PPOX | D38537 | 5 | Pass | 7 80 | 2 05 | 5 | Fail | FALSE | TRUE | FALSE | 4.00 | 1.95 | protoporphymogen | 1q22 | |
| | | | | | | | | | | | | | | oxidase, PPOX | | |
| | | | | | | | | | | | | | | | #N/A | |
| 37149 s at | UNK U95 | U95626 | 4 | Pass | 10 00 | 8.29 | 3 | Fail | FALSE | TRUE | FALSE | 25 00 | −2.50 | lactotransferin, LTF | 3q21-q23 | |
| | | | | | | | | | | | Absent in | | | | | |
| | | | | | | | | | Present | Present | RA, | | Fold | | | |
| | | | | | | Std | sum of | | in RA | in RA, | Present | Avg | Change | | | |
| | | | sum of | 4 of 6 | Avg | Dev | abs | 7 of 13 | and | Absent in | in | Freq | RA/ | | Chromo- | |
| qualifier | name | | abs dec | present | Freq | RA | dec | present | Normal | Normal | Normal | (Normal) | Normal | Name | some | |
| 1029 s at | UNK U07 | U07794 | 0 | Fail | #DIV/ | #DIV/ | 7 | Pass | FALSE | FALSE | TRUE | 2 43 | #DIV/ | TXK tyrosine kinase, TXK | 4p12 | Kinase |
| | | | | | 0¹ | 0¹ | | | | | | | 01 | | | |
| 31495_at | SCYC2 | D63789 | 0 | Fail | #DIV/ | #DIV/ | 7 | Pass | FALSE | FALSE | TRUE | 4 29 | #DIV/ | small inducible cytokine | 1q21- | |
| | | | | | 01 | 0¹ | | | | | | | 01 | subfamily C, member 1 | q25.1q23- | |
| | | | | | | | | | | | | | | (lymphotactin),small | q25 | |
| | | | | | | | | | | | | | | inducible cytokine | | |
| | | | | | | | | | | | | | | subfamily C member 2; | | |
| | | | | | | | | | | | | | | SCYC1 SCYC2 | | |
| 32319_at | TNFSF4 | AL022310 | 0 | Fail | #DIV/ | #DIV/ | 7 | Pass | FALSE | FALSE | TRUE | 10.00 | #DIV/ | tomor necrosis factor | 1q25 | |
| | | | | | 0¹ | 0¹ | | | | | | | 01 | (ligand) superfamily, | | |
| | | | | | | | | | | | | | | member 4 (tax- | | |
| | | | | | | | | | | | | | | transcriptionally activated | | |
| | | | | | | | | | | | | | | glycoprotein 1, 34kD), | | |
| | | | | | | | | | | | | | | TNFSF4 | | |
| 32350_at | MALT1 | AB026118 | 0 | Fail | #DIV/ | #DIV/ | 7 | Pass | FALSE | FALSE | TRUE | 2 43 | #DIV/ | mucosa associated | 18q21 | |
| | | | | | 0¹ | 01 | | | | | | | 01 | lymphoid tissue lymphoma | | |
| | | | | | | | | | | | | | | translocation gene 1, | | |
| | | | | | | | | | | | | | | MALT1 | | |
| 32539 at | COP9 | U51205 | 0 | Fail | ·0DIV/ | #DIV/ | 7 | Pass | FALSE | FALSE | TRUE | 5.29 | #DIV/ | COP9 homolog, COP0 | | |
| | | | | | 0¹ | 0¹ | | | | | | | 01 | | | |
| 33410 at | ITGA6 | S66213 | 0 | Fail | #DIV/ | #DIV/ | 7 | Pass | FALSE | FALSE | TRUE | 2 29 | #DIV/ | integrin, alpha 6, ITGA6, | 2 | |
| | | | | | 01 | 0¹ | | | | | | | 01 | | | |
| 34704_r_at | UNK_AA1 | AA151971 | 0 | Fail | #DIV/ | #DIV/ | 7 | Pass | FALSE | FALSE | TRUE | 3 57 | #DIV/ | HERV-H LTR-associating | 8q24 | |
| | | | | | 01 | 0¹ | | | | | | | 01 | 1, HHLA1 | | |
| 34875_r_at | KIAA0203 | D86958 | 0 | Fail | #DIV/ | #DIV/ | 7 | Pass | FALSE | FALSE | TRUE | 2.57 | #DIV/ | KIAA0203 gene product, | 8 | |
| | | | | | 01 | 0¹ | KIAA0203 | | | | | | | | | |
| 36237_at | SLC22A6 | AB009698 | 0 | Fail | #DIV/ | #DIV/ | 7 | Pass | FALSE | FALSE | TRUE | 3 14 | #DIV/ | solute carrier family 22 | 11q11 | |
| | | | | | 01 | 0¹ | | | | | | | 01 | (organic anton | | |
| | | | | | | | | | | | | | | transporter), member 6, | | |
| 38492_at | KYNU | D55639 | 0 | Fail | #DIV/ | #DIV/ | 7 | Pass | FALSE | FALSE | TRUE | 3.14 | #DIV/ | kynuremnase (L- | | |
| | | | | | 0¹ | 0¹ | kynuruenine hydrolase); | | | | | | | | | |
| | | | | | | | | | | | | | | KYNU | | |
| 38512_r_at | ELAVL3 | D26158 | 0 | Fail | #DIV | #DIV/ | 7 | Pass | FALSE | FALSE | TRUE | 22.71 | #DIV/ | ELAV (embryone lethal, | 19p13 2 | |
| | | | | | 01 | 0¹ | | | | | | | 01 | abnormal vision, | | |
| | | | | | | | | | | | | | | Drosophila)-like 3 (Hu | | |
| | | | | | | | | | | | | | | antigen C), ELAVL3 | | |
| 40590_at | CDC27 | AA166687 | 0 | Fail | #DIV/ | #DIV/ | 7 | Pass | FALSE | FALSE | TRUE | 3 57 | #DIV/ | cell division cycle 27, | 17q12 | |
| | | | | | 0¹ | 0¹ | | | | | | | 01 | CDC27 | 17q23.2 | |
| 526_s_at | PMS2 | U13696 | 0 | Fail | #DIV/ | #DIV/ | 7 | Pass | FALSE | FALSE | TRUE | 2 71 | #DIV/ | postmetotic segregation | 7p22 | |
| | | | | | 0¹ | 01 | | | | | | | 01 | increased (S cerevisiae) 2, | | |
| | | | | | | | | | | | | | | PMS2 | | |
| AFFX-M2783 | 28SRNAM | M27830 | 0 | Fail | #DIV/ | #DIV/ | 7 | Pass | FALSE | FALSE | TRUE | 39 14 | #DIV/ | | | |
| | | | | | 0¹ | 0¹ | | | | | | | 01 | | | |
| 36411_s_at | ELAVL2 | U29943 | 0 | Fail | #DIV/ | #DIV/ | 8 | Pass | FALSE | FALSE | TRUE | 7.25 | #DIV/ | ELAV (embryone lethal, 5p21 | | |
| | | | | | 0¹ | 0¹ | | | | | | | 01 | abnormal vision, | | |
| | | | | | | | | | | | | | | Drosophila)-like 2, | | |
| | | | | | | | | | | | | | | ELAVL2 | | |
| | | | | | | | | | Present | Present | Absent in | Avg | Fold | | | |
| | | | sum of | | Avg | Std | sum of | | in RA | in RA, | Freq | Change | | | | |
| Affy | Affy | | Present | 4 of 6 | Freq | Dev | abs | 7 of 13 | and | Absent in | (Nor- | RA/ | | Chromo- | Kinase or | |
| Qualifier | Name | accession | Calls | present | RA | RA | dec | present | Normal | Normal | mal) | Normal | Name | some | Phosphatase | |
| 38660_at | COX6A2 | F27891 | 0 | Fail | #DIV/ | #DIV/ | 8 | Pass | FALSE | FALSE | TRUE | 2.63 | #DIV/ | cytochrome oxidase | 16p | |
| | | | | | 01 | 01 | | | | | | | 01 | | | |
| 23641_at | KIAA0979 | AB023196 | 0 | Fail | #DIV/ | #DIV/ | 9 | Pass | FALSE | FALSE | TRUE | 2.56 | #DIV/ | KIAA0979 | 13q12- | |
| | | | | | 0¹ | 0¹ | | | | | | | 01 | protein, androgen-induced | q13.13q12 | |
| | | | | | | | | | | | | | | prostate proliferative | 3 | |
| | | | | | | | | | | | | | | shutolf associated protein, | | |
| | | | | | | | | | | | | | | AS3, KIAA0979 | | |
| 32941_at | ICSBP1 | M91196 | 0 | Fail | #DIV/ | #DIV/ | 9 | Pass | FALSE | FALSE | TRUE | 8 22 | #DIV/ | interferon consensus | | |
| | | | | | 01 | 01 | | | | | | | 01 | sequence binding protein | | |
| | | | | | | | | | | | | | | 1, ICSBP1 | | |
| 14_at | ELL2 | U88629 | 1 | Fail | 11 00 | #DIV/ | 7 | Pass | FALSE | FALSE | TRUE | 3 71 | 2.96 | ELL-RELATED RNA | | |
| | | | | | | 01 | | | | | | | | POLYMERASE II, | | |
| | | | | | | | | | | | | | | ELONGATION | | |
| | | | | | | | | | | | | | | FACTOR, ELL2 | | |
| 35590_s_at | CIPR | X81832 | 3 | Fail | 25 33 | 2.89 | 7 | Pass | FALSE | FALSE | TRUE | 9 43 | 2.69 | gastric inhibitory | 19q13 3 | |
| | | | | | | | | | | | | | | polypeptide receptor, | | |
| | | | | | | | | | | | | | | GIPR | | |
| 31599_at | SLC13A2 | U26209 | 3 | Fail | 11 67 | 2.08 | 7 | Pass | FALSE | FALSE | TRUE | 4 43 | 2.63 | solute carrier family 13 | 17p11 1- | |
| | | | | | | | | | | | | | | (sodium-dependent | q11 1 | |
| | | | | | | | | | | | | | | dicarboxylate transporter)- | | |
| | | | | | | | | | | | | | | member 2 SLC13A2 | | |
| 34903 at | UNK AI0 | AI017382 | 3 | Fail | 15 67 | 7.09 | 9 | Pass | FALSE | FALSE | TRUE | 6 22 | 2.52 | | | |
| 36205_at | NDUFA9 | L04490 | 1 | Fail | 11.00 | #DIV/ | 7 | Pass | FALSE | FALSE | TRUE | 4.43 | 2.48 | NADH dehydrogenase | 12p13 3 | |
| | | | | | | 0¹ | | | | | | | | (ubiquinone) 1 alpha | | |
| | | | | | | | | | | | | | | subcomplex, 9 (39kD), | | |
| | | | | | | | | | | | | | | NDUFA9 | | |
| 32048 at | UNK AL0 | AL049075 | 3 | Fail | 11 33 | 3 79 | 8 | Pass | FALSE | FALSE | TRUE | 4 63 | 2.45 | | | |
| 37521_s_at | HSA6591 | H82458 | 1 | Fail | 10.00 | #DIV/ | 7 | Pass | FALSE | FALSE | TRUE | 4 14 | 2.41 | nuclear cystein-rich | | |
| | | | | | | 0¹ | | | | | | | | protein, HSA6591 | | |
| 396_f_at | EPOR | X97671 | 2 | Fail | 29.50 | 0 71 | 9 | Pass | FALSE | FALSE | TRUE | 12 44 | 2.37 | cythroprotein receptor, | 19p13 3- | |
| | | | | | | | | | | | | | | EPOR | p13 2 | |
| 34098 f at | UNK AI79 | AI799757 | 2 | Fail | 7 50 | 2 12 | 7 | Pass | FALSE | FALSE | TRUE | 3 29 | 2.28 | | | |
| 31594_at | KRTHA3A Y16788 | 2 | Fail | 11 00 | 5 66 | 7 | Pass | FALSE | FALSE | TRUE | 4.86 | 2.26 | keratin, hair, acidic,3A, | 17q12- | | |
| | | | | | | | | | | | | | | KRTHA3A | q21 | |
| 40651_s_at | CRHR1 | AF039523 | 1 | Fail | 7.00 | #DIV/ | 10 | Pass | FALSE | FALSE | TRUE | 3 10 | 2.26 | corticotropin releasing | 17q12- | |
| | | | | | | 01 | | | | | | | | hormone receptor 1, | q22 | |
| | | | | | | | | | | | | | | CRHR1 | | |
| 40299_at | RE2 | AF091890 | 2 | Fail | 35 50 | 13.44 | 8 | Pass | FALSE | FALSE | TRUE | 15.75 | 2.25 | G-protein coupled | | |
| | | | | | | | | | | | | | | receptor, RE2 | | |
| 33037_at | UNK_AL0 | AL022165 | 1 | Fail | 7.00 | #DIV/ | 8 | Pass | FALSE | FALSE | TRUE | 3.13 | 2.24 | chondraium 6- | Xp11 | |
| | | | | | | 01 | | | | | | | | sulfotransferase-2, C6ST-2 | | |
| 39920 r at | CRF | AF095154 | 3 | Fail | 93 33 | 14 05 | 10 | Pass | FALSE | FALSE | TRUE | 41 70 | 2.24 | Clq-related factor, CRF | 17q21 | |
| 31586 f at | UNK X72 | X72475 | 3 | Fail | 8 67 | 2.89 | 9 | Pass | FALSE | FALSE | TRUE | 3.89 | 2.23 | | | |
| 37488_at | FNTB | L00635 | 2 | Fail | 7 00 | 1.41 | 7 | Pass | FALSE | FALSE | TRUE | 3 14 | 2.23 | famesyltransferase, CAAX | 14q23- | |
| | | | | | | | | | | | | | | box, beta, FNTB | q24 | |
| 2047 s at | JUP M23410 | 3 | Fail | 17.33 | 9 29 | 8 | Pass | FALSE | FALSE | TRUE | 7 88 | 2.20 | junction plakuglobin, JUP | 17q21 | | |
| 898_s_at | GDF1 | M62302 | 3 | Fail | 10 33 | 0 58 | 8 | Pass | FALSE | FALSE | TRUE | 4 75 | 2.18 | growth differentiation 19p12 | | |
| | | | | | | | | | | | | | | factor 1, GDF1 | | |
| 40622 r at | UNK AL0 | AL096740 | 3 | Fail | 38 00 | 13 11 | 9 | Pass | FALSE | FALSE | TRUE | 17 56 | 2.16 | | | |
| 41324 g at | FOXM1 | U90917 | 1 | Fail | 16 00 | #DIV/ | 10 | Pass | FALSE | FALSE | TRUE | 7.50 | 2.13 | forkhead box M1, FOXM1 | 12p13 | |
| | | | | | | 0¹ | | | | | | | | | | |
| 39844 at | UNK AI80 | AI806379 | 2 | Fail | 8 00 | 2 83 | 12 | Pass | FALSE | FALSE | TRUE | 3 83 | 2.09 | | | |
| 34815_at | TNRC12 | U80743 | 1 Fail | 5 00 | #DIV/ | 7 | Pass | FALSE | FALSE | TRUE | 2 43 | 2.06 | trinucleotide repeat | 12qter | | |
| | | | | | | 01 | | | | | | | | containing 12, TNRC12 | | |
| 33493_at | HFL-EDD | AF048849 | 2 | Fail | 10 00 | 2.83 | 8 | Pass | FALSE | FALSE | TRUE | 4 88 | 2.05 | erythroid diferentiation | | |
| | | | | | | | | | | | | | | and denucleation factor 1; | | |
| | | | | | | | | | | | | | | HFL-EDDG 1 | | |
| 1581_s_at | UNK_M27 | M27504 | 1 | Fail | 5.00 | #DIV/ | 9 | Pass | FALSE | FALSE | TRUE | 2.44 | 2.88 | topoisomerase (DNA) II | 3p24 | |
| | | | | | | 0¹ | | | | | | | | beta (180kD), TOP2B | | |
| 33697_at | F2RX7 | Y12851 | 3 | Fail | 7 00 | 1.00 | 8 | Pass | FALSE | FALSE | TRUE | 3.50 | 2.00 | punergic receptor P2X, | 12q24 | |
| | | | | | | | | | | | | | | ligand-gated ion channel, | | |
| | | | | | | | | | | | | | | 7; P2RX7 | | |
| 31503 at | UNK W28 | W28732 | 2 | Fail | 9 50 | 7.78 | 11 | Pass | FALSE | FALSE | TRUE | 4 82 | 1.97 | | | |
| 718_at | PRSS11 | D87258 | 2 | Fail | 4.50 | 0.71 | 7 | Pass | FALSE | FALSE | TRUE | 2.29 | 1.97 | protease, serine, 11 (IGF | 10q25.3- | |
| | | | | | | | | | | | | | | binding); PRSS11 | q26.2 | |
| 38229 at | UNK X90 | X90579 | 3 | Fail | 92.33 | 20 98 | 7 | Pass | FALSE | FALSE | TRUE | 47 00 | 1.96 | | | |
| 393_at | RUNX1 | X90976 | 2 | Fail | 5 00 | 0 00 | 11 | Pass | FALSE | FALSE | TRUE | 2.55 | 1.96 | runt-related transcription | 21q22 3 | |
| | | | | | | | | | | | | | | factor 1 (acute myeloid | | |
| | | | | | | | | | | | | | | leukemia 1, am11 | | |
| | | | | | | | | | | | | | | oncogene), RUNX1 | | |
| 1170 at | CSF1 | M37435 | 1 | Fail | 9 00 | #DIV/ | 10 | Pass | FALSE | FALSE | TRUE | 4.60 | 1.96 | | | |
| | | | | | | 0¹ | | | | | | | | | | |
| | | | | | | | | | | | | | | | #N/A | |
| 40490_at | DDX21 | U41387 | 3 | Fail | 3 67 | 1.53 | 11 | Pass | FALSE | FALSE | TRUE | 7.82 | −2.13 | DEAD/H (Asp-Gln-Ala- | 10 | |
| | | | | | | | | | | | | | | Asp/His) box polypeptide | | |
| | | | | | | | | | | | | | | 21, DDX21 | | |

TABLE 4
CIA PBMC data
Fold changes are shown as normalized

| | | | | | | | | TBS- | | | | | | | | |
|---|---|---|---|---|---|---|---|---|---|---|---|---|---|---|---|---|
| TBS O* | | Preart | TBS- | | | TBS- | TBS- | 7- | | | | | | | | |
| Systematic | Control | hritic | 1 | TBS-3 | TBS-4 | 5 | 6 | 9 | Common | Genbank | EC | Description | Phenotype | Map | Keywords | Symbol |
| J03023 | 0 | −1.62 | 1.08 | −3.25 | 2.23 | 1.54 | 1.54 | 1.62 | hemopoietic | J03023 | 2.7.1.112 | TYROSINE- | EXPRESSED | 2 86.0 cM | Hemostasis | Hck |
| | | | | | | | | | cell kinase; | | | PROTEIN | PREDOMINA | | | |
| | | | | | | | | | Hck | KINASE HCK | NTLY IN | | | | | |
| | | | | | | | | | | | | (EC2.7.1.112) | CELLS OF | | | |
| | | | | | | | | | | | | (P56-HCK | THE | | | |
| | | | | | | | | | | | | AND P60- | MYELOID | | | |
| | | | | | | | | | | | | HCK) | AND B- | | | |
| | | | | | | | | | | | | (HEMOPOJET | LYMPHOID | | | |
| | | | | | | | | | | | | IC CELL | LINEAGES | | | |
| | | | | | | | | | | | | KINASE) (B- | | | | |
| | | | | | | | | | | | | CELL/MYELO | | | | |
| | | | | | | | | | | | | ID KINASE) | | | | |
| AA028657 | 0 | 3.00 | 19.00 | 17.00 | 17.00 | 37.50 | 16 50 | 20.00 | EST; Unknown | AA028657 | | | | | EST; Unknown | Unknown |
| Msa.10146.0 | 0 | 1.80 | 12.40 | 13.80 | 13.60 | 19.60 | 15.20 | 19.20 | vWF,human | AA168633 | | | | | Hemostasis | |
| Msa 10146.0 | 0 | 1 80 | 12.40 | 13.80 | 13.60 | 19,60 | 15.20 | 19.20 | vWF, human | AA168633 | | | | | Hemostasis | |
| W62701 | 0 | 2.40 | 20.20 | 19.00 | 16.40 | 22.80 | 18.20 | 18.80 | | W62701 | | | | | | |
| W62701 | 0 | 2.40 | 20.20 | 19.00 | 16.40 | 22.80 | 18.20 | 18.80 | | W62701 | | | | | | |
| Msa.1497.0 | 0 | 1.50 | 7.00 | 5.50 | 6.00 | 11.00 | 6.50 | 15.50 | calmodulin 3; | M19380 | 2.7.1.38 | | | 7 4.0 cM | Regulatory | Calm3 |
| | | | | | | | | | Calm3 | | | | | | | |
| Msa 1497.0 | 0 | 1.50 | 7.00 | 5.50 | 6.00 | 11.00 | 6.50 | 15.50 | calmodulin 3; | M19380 | 2.7.1.38 | | | 7 4.0 cM | Regulatory | Calm3 |
| | | | | | | | | | Calm3 | | | | | | | |
| Msa.723.0 | 0 | 2.00 | 8.00 | 5.00 | 3.00 | 10.00 | 9.00 | 15.00 | aquaporin 1; | L02914 | | AQUAPORIN- | ERYTHROCY | 6 27.0 cM | Cell Surface | Aqp1 |
| | | | | | | | | | Aqp1 | | | CHIP | TES AND | | Protein | |
| | | | | | | | | | | | | (WATER | RENAL | | | |
| | | | | | | | | | | | | CHANNEL | TUBULES. | | | |
| | | | | | | | | | | | | PROTEIN | | | | |
| | | | | | | | | | | | | FOR RED | | | | |
| | | | | | | | | | | | | BLOOD | | | | |
| | | | | | | | | | | | | CELLS AND | | | | |
| | | | | | | | | | | | | KIDNEY | | | | |
| | | | | | | | | | | | | PROXIMAL | | | | |
| | | | | | | | | | | | | TUBULE) | | | | |
| | | | | | | | | | | | | (AQUAPORIN | | | | |
| | | | | | | | | | | | | 1) (EARLY | | | | |
| | | | | | | | | | | | | RESPONSE | | | | |
| D16262 | 0 | 0.00 | 9.00 | 10.00 | 8.00 | 21.00 | 16.00 | 14.00 | mesodems | D16262 | | | | 6 7.5 cM | Cytokine | Mest |
| | | | | | | | | | specific | | | | | | | |
| | | | | | | | | | transcript; | | | | | | | |
| | | | | | | | | | Mest | | | | | | | |
| X15592 | 0 | 1.33 | 13.67 | 13.00 | 16.67 | 14.67 | 13.00 | 14.00 | cytotoxic T | X15592 | | CTLA-2-BETA | | 13 42.0 cM | | Ctla2b |
| | | | | | | | | | lymphocyte- | | | PROTEIN | | | | |
| | | | | | | | | | associated | | | PRECURSOR | | | | |
| | | | | | | | | | protein 2 beta; | | | (FRAGMENT) | | | | |
| | | | | | | | | | Ct1a2b | | | | | | | |
| Msa.3237.0 | 0 | 0.00 | 7.50 | 9.00 | 9.00 | 9.50 | 9.50 | 13.50 | four and a half | W14830 | | | | X A6-A7.1 | Regulatory | Fh11 |
| | | | | | | | | | LIM domains | | | | | | | |
| | | | | | | | | | 1; Fh11 | | | | | | | |
| M87276 | 0 | 0.00 | 6.00 | 7.00 | 5.00 | 10.00 | 11.00 | 13.00 | thrombospondi | M87276 | | THROMBOSP | | 2 65.0 cM | Extracellular | Thbs1 |
| | | | | | | | | | n 1; Thbs1 | | | ONDIN 1 | | | Protein | |
| | | | | | | | | | | | | PRECURSOR. | | | | |
| AA616664 | 0 | 0.00 | 10.75 | 11.00 | 10.00 | 16.75 | 12.00 | 11.25 | | AA616664 | | | | | | |
| W45778 | 0 | 1.27 | 8.47 | 9.40 | 9.00 | 9.53 | 8.00 | 9.27 | von Willebrand | W45778 | | | | | Hemostasis | vWF |
| | | | | | | | | | Factor; vWF; | | | | | | | |
| | | | | | | | | | homolog | | | | | | | |
| W64688 | 0 | 0.00 | 6.82 | 7.36 | 5.55 | 9.82 | 7.09 | 8.73 | | W64688 | | | | | EST; Unknown | |
| ab000822 | 0 | 1.10 | 7.30 | 7.10 | 7.80 | 10.10 | 7.70 | 8.50 | synaptosomal- | AB000822 | | | | 2 61.8 cM | | Snap23 |
| | | | | | | | | | associated | | | | | | | |
| | | | | | | | | | protein, 23kD; | | | | | | | |
| | | | | | | | | | Snap23 | | | | | | | |
| M19380 | 0 | 0.00 | 4.67 | 4.00 | 5.33 | 9.00 | 6.67 | 8.33 | calmodulin 3; | M19380 | 2.7.1.38 | | | 7 4.0 cM | Regulatory | Calm3 |
| | | | | | | | | | Calm3 | | | | | | | |
| AA542220 | 0 | −1 09 | 5.00 | 4.75 | 7.00 | 8.92 | 5.92 | 7.50 | TBX1 protein; | AA542220 | TBX1 | TESTIS | | Intracellular | TBX1 | |
| | | | | | | | | | | | | TBX1 | | | PROTEIN (T- | SPECIFIC. |
| | | | | | | | | | | | | BOX | | | | |
| | | | | | | | | | | | | PROTEIN 1) | | | | |
| | | | | | | | | | | | | (TESTIS- | | | | |
| | | | | | | | | | | | | SPECIFIC T- | | | | |
| | | | | | | | | | | | | BOX | | | | |
| Msa 1160 0 | 0 | 0.00 | 7.67 | 2.00 | 7.67 | 2.00 | 5.33 | 5.67 | serum amyloid | X03505 | | SERUM | FOUND IN | 7 23.5 cM | Extracellular | Saa3 |
| | | | | | | | | | A 3; Saa3 | | | AMYLOID A- | VARIOUS | | | |
| | | | | | | | | | | | | 3 PROTEIN | TISSUES | | | |
| | | | | | | | | | | | | PRECURSOR. | | | | |
| X03479 | 0 | −1.20 | 6.17 | 1.50 | 5.17 | 1.67 | 3.50 | 5.17 | serum amyloid | X03479 | | SERUM | FOUND IN | 7 23.5 cM | Extracellular | Saa3 |
| | | | | | | | | | A 3; Saa3 | | | AMYLOID A- | VARIOUS | | | |
| | | | | | | | | | | | | 3 PROTEIN | TISSUES | | | |
| | | | | | | | | | | | | PRECURSOR. | | | | |
| U92478 | 0 | 0.00 | 3.00 | 2.33 | 3.33 | 6.00 | 5.33 | 4.67 | development | U92478 | | | | | Regulatory | Ddef1 |
| | | | | | | | | | and | | | | | | | |
| | | | | | | | | | differentiation | | | | | | | |
| | | | | | | | | | enhancing; | | | | | | | |
| | | | | | | | | | Ddef1 | | | | | | | |
| Msa.3665.0 | 0 | −1.12 | 3.33 | 4.00 | 3.67 | 5.44 | 4.67 | 4.67 | DNA segment, | AA116604 | | | | 2 24.0 cM | Proteolytic | D2Wsu143c |
| | | | | | | | | | Chr 2, Wayne | | | | | | | |
| | | | | | | | | | State | | | | | | | |
| | | | | | | | | | University 143, | | | | | | | |
| | | | | | | | | | expressed; | | | | | | | |
| | | | | | | | | | D2Wsu143e | | | | | | | |
| D67016 | 0 | −2.33 | −1.75 | −1.75 | 1.14 | 3.14 | 1.14 | 4.43 | heat shock | D67016 | | HEAT-SHOCK | FOUND IN | 5 88.0 cM | | Hsp105 |
| | | | | | | | | | protein, 105 | | | PROTEIN 105 | MOST | | | |
| | | | | | | | | | kDa; Hsp105 | | | KDA (HEAT | TISSUES. | | | |
| | | | | | | | | | | | | SHOCK- | HIGHLY | | | |
| | | | | | | | | | | | | RELATED 100 | EXPRESSED | | | |
| | | | | | | | | | | | | KDA | IN BRAIN. | | | |
| | | | | | | | | | | | | PROTEIN E71) | | | | |
| | | | | | | | | | | | | (HSP-E71) | | | | |
| | | | | | | | | | | | | (HEAT | | | | |
| | | | | | | | | | | | | SHOCK 110 | | | | |
| | | | | | | | | | | | | KDA | | | | |
| | | | | | | | | | | | | PROTEIN) (42 | | | | |
| AA120653 | 0 | −1.69 | 2.84 | 2.41 | 2.77 | 4.50 | 3.07 | 3.80 | transgelin 2; | AA120653 | | TRANSGELIN | | 1 94.2 cM | Structural | Tagln1 |
| | | | | | | | | | Tagln2 | | | 2. | | Protein | | |
| AA285502 | 0 | −2.00 | 2.00 | 2.00 | 2.00 | 4.50 | 3.50 | 3.75 | receptor | AA285502 | | | | | Regulatory | Ramp1 |
| | | | | | | | | | (calcitonin) | | | | | | | |
| | | | | | | | | | activity | | | | | | | |
| | | | | | | | | | modifying | | | | | | | |
| | | | | | | | | | protein 1; | | | | | | | |
| | | | | | | | | | Ramp1 | | | | | | | |
| U35124 | 0 | −1.87 | 2.07 | 2.00 | 2.27 | 4.00 | 2.73 | 3.67 | protein | U35124 | | | | 1 17.3 cM | Regulatory | Ptpn18 |
| | | | | | | | | | tyrosine | | | | | | | |
| | | | | | | | | | phosphatase, | | | | | | | |
| | | | | | | | | | non-receptor | | | | | | | |
| | | | | | | | | | type 18; | | | | | | | |
| | | | | | | | | | Ptpn18 | | | | | | | |
| U05837 | 0 | −1.60 | 0.00 | −1.14 | 1.75 | 2.25 | 2.00 | 3.50 | hexosaminidas | U05837 | 3.2.1.52 | BETA- | | 9 29.0 cM | Proteolytic | Hexa |
| | | | | | | | | | e A; Hexa | | | HEXOSAMINI | | | | |
| | | | | | | | | | | | | DASE ALPHA | | | | |
| | | | | | | | | | | | | CHAIN | | | | |
| | | | | | | | | | | | | PRECURSOR | | | | |
| | | | | | | | | | | | | (EC 3.2.1.52) | | | | |
| | | | | | | | | | | | | (N-ACETYL- | | | | |
| | | | | | | | | | | | | BETA- | | | | |
| | | | | | | | | | | | | GLUCOSAMI | | | | |
| | | | | | | | | | | | | NIDASE) | | | | |
| | | | | | | | | | | | | (BETA-N- | | | | |
| | | | | | | | | | | | | ACETYLHEX | | | | |
| | | | | | | | | | | | | OSAMINIDAS | | | | |
| | | | | | | | | | | | | E) | | | | |
| U27830 | 0 | −1.67 | −2.50 | −2.50 | 0.00 | 2.00 | 1.20 | 3.20 | stress-induced | U27830 | | | | | | Stip1 |
| | | | | | | | | | phosphoprotein | | | | | | | |
| | | | | | | | | | 1; Stip1 | | | | | | | |
| Msa.4113.0 | 0 | −1.50 | 1.44 | 1.22 | 2.78 | 1.33 | 2.44 | 3.11 | glucocortucoid- | AA050733 | | | | | Signal | Gilz |
| | | | | | | | | | induced | | | | | | Transduction | |
| | | | | | | | | | leucine zipper; | | | | | | | |
| | | | | | | | | | Gilz | | | | | | | |
| C79010 | 0 | −1.33 | 2.25 | 2.08 | 4.33 | 4.25 | 3.00 | 3.08 | Src-associated | C79010 | | | | | ?? | Saps |
| | | | | | | | | | adaptor | | | | | | | |
| | | | | | | | | | protein; Saps | | | | | | | |
| C79010 | 0 | −1.33 | 2.25 | 2.08 | 4.33 | 4.25 | 3.00 | 3.08 | Src-associated | C79010 | | | | | ?? | Saps |
| | | | | | | | | | adaptor | | | | | | | |
| | | | | | | | | | protein; Saps | | | | | | | |
| Msa.16995.0 | 0 | −1.27 | 2.79 | 2.64 | 5.29 | 3.07 | 2.57 | 3.04 | arachidonate 5- | W83564 | | | | | Intracellular | |
| | | | | | | | | | lipoxygenase | | | | | | Protein | |
| | | | | | | | | | activating | | | | | | | |
| | | | | | | | | | protein | | | | | | | |
| M22479 | 0 | −2.00 | 2.75 | 3.00 | 3.00 | 5.00 | 3.50 | 3.00 | tropomyosin 2, | M22479 | | | | | EST; Unknown | Tpm2 |
| | | | | | | | | | beta; Tpm2 | | | | | | | |
| AA238483 | 0 | −3.33 | −2.00 | −2.00 | −1.25 | −1.11 | −1.11 | 2.90 | CD8 antigen, | AA238483 | | T-CELL | | 6 30.5 cM | | Cd8b |
| | | | | | | | | | beta chain; | | | SURFACE | | | | |
| | | | | | | | | | Cd8b | | | GLYCOPROT | | | | |
| | | | | | | | | | | | | EIN CD8 | | | | |
| | | | | | | | | | | | | BETA CHAIN | | | | |
| | | | | | | | | | | | | PRECURSOR | | | | |
| | | | | | | | | | | | | (T-CELL | | | | |
| | | | | | | | | | | | | SURFACE | | | | |
| | | | | | | | | | | | | GLYCOPROT | | | | |
| | | | | | | | | | | | | EIN LYT-3) (T- | | | | |
| | | | | | | | | | | | | CELL | | | | |
| | | | | | | | | | | | | MEMBRANE | | | | |
| | | | | | | | | | | | | GLYCOPROT | | | | |
| M27960 | 0 | −2.00 | 1.17 | −2.00 | 3.50 | 1.33 | 1.33 | 2.83 | interleukin 4 | M27960 | | INTERLEUKI | | 7 62.0 cM | Receptor | I14ra |
| | | | | | | | | | receptor, alpha; | | | N-4 | | | | |
| | | | | | | | | | I14ra | | | RECEPTOR | | | | |
| | | | | | | | | | | | | ALPHA | | | | |
| | | | | | | | | | | | | CHAIN | | | | |
| AA033103 | 0 | −1.33 | 1.75 | 1.50 | 2.75 | 3.25 | 1.50 | 2.75 | | AA033103 | | | | | | |
| AA261246 | 0 | −1.47 | 2.71 | 2.61 | 4.14 | 4.57 | 2.89 | 2.71 | | AA261246 | | | | | | |
| AA104254 | 0 | −6.00 | −6.00 | −6.00 | −3.00 | −6.00 | −1.20 | 2.67 | transcription | AA104254 | | | | | Transcription | KID-1 |
| | | | | | | | | | factor 17; | | | | | | | |
| | | | | | | | | | protein kinase | | | | | | | |
| | | | | | | | | | KID-1 (kinase | | | | | | | |
| | | | | | | | | | induced by | | | | | | | |
| | | | | | | | | | depolarization) | | | | | | | |
| | | | | | | | | | ; rat | | | | | | | |
| X15591 | 0 | 1.03 | 3.05 | 2.89 | 3.02 | 1.95 | 2.10 | 2.63 | cytotoxic T | X15591 | | CTLA-2- | | 13 36.0 cM | Cell Surface | Ct1a2a |
| | | | | | | | | | lymphocyte- | | | ALPHA | | | Protein | |
| | | | | | | | | | associated | | | PROTEIN | | | | |
| | | | | | | | | | protein 2 | | | PRECURSOR. | | | | |
| | | | | | | | | | alpha; Ct1a2a | | | | | | | |
| U88328 | 0 | −1.33 | 1.88 | 1.50 | 4.88 | 2.38 | 1.50 | 2.63 | cytokine | U88328 | | CYTOKINE | | | ECM (Matrix | Cish3 |
| | | | | | | | | | inducible SH2- | | | INDUCIBLE | | | Prot) | |
| | | | | | | | | | containing | | | SH2- | | | | |
| | | | | | | | | | protein 3; | | | CONTAINING | | | | |
| | | | | | | | | | Cish3 | | | PROTEIN 3 | | | | |
| | | | | | | | | | | | | (PROTEIN EF- | | | | |
| | | | | | | | | | | | | 10). | | | | |
| Msa.4067.0 | 0 | −1.25 | 1.20 | 1.80 | 3.20 | 2.40 | 1.20 | 2.60 | Sip1? | AA003876 | | | | | Unknown | |
| AA289661 | 0 | −1.75 | 1.90 | 2.05 | 3.86 | 3.76 | 2.00 | 2.57 | EST, Unknown | AA289661 | | | | | EST; Unknown | Unknown |
| AF003693 | 0 | −1.39 | 2.56 | 2.00 | 3.36 | 3.64 | 2.92 | 2.56 | syndecan | AF003693 | | | | | | Sdcbp |
| | | | | | | | | | binding | | | | | | | |
| | | | | | | | | | protein; Sdcbp | | | | | | | |
| AA184116 | 0 | −2.00 | 2.50 | 3.00 | 4.30 | 3.80 | 3.20 | 2.50 | homolog of | AA184116 | | | | | Structural | |
| | | | | | | | | | Alpha-actinin | | | | | | Protein | |
| | | | | | | | | | (human) | | | | | | | |
| U71205 | 0 | 0.00 | 3.00 | 3.00 | 4 00 | 5.50 | 3.00 | 2.50 | RAS-like | U71205 | | | | | TRanscription | Rit |
| | | | | | | | | | protein | | | | | | | |
| | | | | | | | | | expressed in | | | | | | | |
| | | | | | | | | | many tissues; | | | | | | | |
| | | | | | | | | | Rit | | | | | | | |
| Msa.36175.0 | 0 | −1.33 | 2.50 | 2.50 | 16.00 | 2.50 | 2.25 | 2.50 | | AA124453 | | | | | EST; Unknown | |
| Msa.22134.0 | 0 | 0.00 | 1.50 | 0.00 | 7.50 | 1.50 | 1 50 | 2 50 | Unknown | AA031158 | | | | | EST; Unknown | |
| X56602 | 0 | 0.00 | 8.50 | 1.50 | 1.50 | 0.00 | 1.50 | 2.50 | interferon- | X56602 | | UBIQUITIN | | | Cytokine | Isg15 |
| | | | | | | | | | stimulated | | | CROSS- | | | | |
| | | | | | | | | | protein (15 | | | REACTIVE | | | | |
| | | | | | | | | | kDa); Isg15 | | | PROTEIN | | | | |
| | | | | | | | | | | | | (INTERFERO | | | | |
| | | | | | | | | | | | | N- | | | | |
| | | | | | | | | | | | | STIMULATE | | | | |
| U19118 | 0 | −2.00 | −1.33 | −4.00 | −1.33 | 0.00 | 1.50 | 2.50 | activating | U19118 | | CYCLIC-AMP- | | | | Atf3 |
| | | | | | | | | | transcription | | | DEPENDENT | | | | |
| | | | | | | | | | factor 3; Atf3 | | | TRANSCRIPT | | | | |
| | | | | | | | | | | | | ION FACTOR | | | | |
| | | | | | | | | | | | | ATF-3 | | | | |
| | | | | | | | | | | | | (ACTIVATIN | | | | |
| | | | | | | | | | | | | G | | | | |
| | | | | | | | | | | | | TRANSCRIPT | | | | |
| | | | | | | | | | | | | ION FACTOR | | | | |
| | | | | | | | | | | | | 3) | | | | |
| | | | | | | | | | | | | (TRANSCRIP | | | | |
| AA123934 | 0 | 2.67 | −2.00 | −4.00 | 0.00 | 1.38 | 1.25 | 2.38 | EST; Unknown | AA123934 | | | | | EST; Unknown | Unknown |
| L28177 | 0 | −1.20 | 2.17 | 2.33 | 4.50 | 2.83 | 2.67 | 2.33 | DNA-damage | L28177 | | GROWTH | | 3 70.5 cM | Intracellular | Ddit1 |
| | | | | | | | | | inducible | | | ARREST AND | | | Protein | |
| | | | | | | | | | transcript 1; | | | DNA- | | | | |
| | | | | | | | | | Dditl; | | | DAMAGE- | | | | |
| | | | | | | | | | (GADD45a) | | | INDUCIBLE | | | | |
| | | | | | | | | | | | | PROTEIN | | | | |
| Msa641.O | 0 | −1.49 | 2.11 | 1.91 | 3.47 | 2.27 | 1.92 | 2.31 | Fc receptor, | W41745 | | HIGH | | 1 93.3 cM | Receptor | Fcer1g |
| | | | | | | | | | IgE, high | | | AFFINITY | | | | |
| | | | | | | | | | affinity I, | | | IMMUNOGLO | | | | |
| | | | | | | | | | gamma | | | BULIN | | | | |
| | | | | | | | | | polypeptide; | | | EPSILON | | | | |
| | | | | | | | | | Fcer1g | | | RECEPTOR | | | | |
| | | | | | | | | | | | | GAMMA- | | | | |
| | | | | | | | | | | | | SUBUNIT | | | | |
| | | | | | | | | | | | | PRECURSOR | | | | |
| | | | | | | | | | | | | (FCERI) (IGE | | | | |
| | | | | | | | | | | | | FC | | | | |
| | | | | | | | | | | | | RECEPTOR, | | | | |
| | | | | | | | | | | | | GAMMA- | | | | |
| | | | | | | | | | | | | SUBUNIT) | | | | |
| | | | | | | | | | | | | (FC-EPSILON | | | | |
| AA033074 | 0 | −1.43 | 2.30 | 1.40 | 3.10 | 3.10 | 1.70 | 2.30 | flotillin 1; | A.A033074 | | | | | | Flot1 |
| | | | | | | | | | Flot1 | | | | | | | |
| U73004 | 0 | −1.14 | 1.91 | 2.18 | 3.48 | 3.06 | 2.27 | 2.15 | secretory | U73004 | | ANTILEUKOP | HIGHEST | | Proteolytic | Slpi |
| | | | | | | | | | leukocyte | | | ROTEINASE 1 | EXPRESSION | | | |
| | | | | | | | | | protease | | | PRECURSOR | IN LUNG | | | |
| | | | | | | | | | inhibitor; Sipi | | | (ALP) | SPLEEN | | | |
| | | | | | | | | | | | | (SECRETORY | INTESTINE | | | |
| | | | | | | | | | | | | LEUKOCYTE | AND | | | |
| | | | | | | | | | | | | PROTEASE | EPIDIDYMIS | | | |
| | | | | | | | | | | | | INHIBITOR). | WITH | | | |
| | | | | | | | | | | | | | LOWER | | | |
| | | | | | | | | | | | | | LEVELS IN | | | |
| | | | | | | | | | | | | | LIVER AND | | | |
| | | | | | | | | | | | | | SEMINAL | | | |
| | | | | | | | | | | | | | VESICLE. NO | | | |
| | | | | | | | | | | | | | EXPRESSION | | | |
| | | | | | | | | | | | | | IN BRAIN | | | |
| | | | | | | | | | | | | | HEART | | | |
| | | | | | | | | | | | | | KIDNEY AND | | | |
| D37837 | 0 | −1.20 | 0.00 | −1.29 | 3.39 | 1.28 | 1.39 | 2.06 | plastin 2, L; | D37837 | | L-PLASTIN | | | Structural | P1s2 |
| | | | | | | | | | P1s2 | | | (LYMPHOCY | | | Protein | |
| | | | | | | | | | | | | TE | | | | |
| | | | | | | | | | | | | CYTOSOLIC | | | | |
| | | | | | | | | | | | | PROTEIN 1) | | | | |
| | | | | | | | | | | | | (LCP-1) (65 | | | | |
| | | | | | | | | | | | | KDA | | | | |
| | | | | | | | | | | | | MACROPHAG | | | | |
| | | | | | | | | | | | | E PROTEIN) | | | | |
| X81627 | 0 | 1.43 | 4.86 | 3.14 | 17.29 | 3.14 | 2.14 | 2.00 | lipocalin 2; | X81627 | | NEUTROPHIL | | 2 27.0 cM | Extracellular | Lcn2 |
| | | | | | | | | | Lcn2 | | | GELATINASE- | | | Protein | |
| | | | | | | | | | | | | ASSOCIATED | | | | |
| | | | | | | | | | | | | LIPOCALIN | | | | |
| | | | | | | | | | | | | PRECURSOR | | | | |
| | | | | | | | | | | | | (NGAL) (P25) | | | | |
| | | | | | | | | | | | | (SV-40 | | | | |
| | | | | | | | | | | | | INDUCED | | | | |
| | | | | | | | | | | | | 24P3 | | | | |
| Msa.2129.0 | 0 | 2.00 | 6.33 | 3.67 | 31.67 | 3.33 | 2.00 | 2.00 | lipocalin 2; | W13166 | | NEUTROPHIL | | 2 27.0 cM | Extracellular | Lcn2 |
| | | | | | | | | | Lcn2 | | | GELATINASE- | | | Protein | |
| | | | | | | | | | | | | ASSOCIATED | | | | |
| | | | | | | | | | | | | LIPOCALIN | | | | |
| | | | | | | | | | | | | PRECURSOR | | | | |
| | | | | | | | | | | | | (NGAL) (P25) | | | | |
| | | | | | | | | | | | | (SV-40 | | | | |
| | | | | | | | | | | | | INDUCED | | | | |
| | | | | | | | | | | | | 24P3 | | | | |
| U96687 | 0 | 0.00 | 1.47 | −1.25 | 3.07 | 1.73 | 1.53 | 2.00 | paired-Ig-like | U96687 | | | | 7 | | Pira10,Pira16 |
| | | | | | | | | | receptor | | | | | | | |
| | | | | | | | | | A10,paired-Ig- | | | | | | | |
| | | | | | | | | | like receptor | | | | | | | |
| | | | | | | | | | A6; | | | | | | | |
| | | | | | | | | | Pira10,Pira6 | | | | | | | |
| U27838 | 0 | −3 50 | −1.40 | −3.50 | 0.00 | 1.57 | 1.29 | 2.00 | GPI-anchored | U27838 | | | | | | Gpiap-pending |
| | | | | | | | | | membrane | | | | | | | |
| | | | | | | | | | protein 1; | | | | | | | |
| | | | | | | | | | Gpiap-pending | | | | | | | |
| Msa 3234.0 | 0 | −1.33 | −2.00 | −4.00 | 2.75 | 1.25 | 0.00 | 2.00 | myosin If; | X97650 | | | | 17 17.5 cM | Structural | Myo1f |
| | | | | | | | | | Myo1f | | | | | | | |
| AA032906 | 0 | −9.00 | −9.00 | −9.00 | −4.50 | −1.50 | −1.29 | 2.00 | Homologous to | AA032906 | | | | | Patented; | |
| | | | | | | | | | GENESEQN:V | | | | | | Novel | |
| | | | | | | | | | 49566 (human) | | | | | | | |
| AA032906 | 0 | −9.00 | −9.00 | −9.00 | −4.50 | −1.50 | −1.29 | 2.00 | Homologous to | AA032906 | | | | | Patented; | |
| | | | | | | | | | GENESEQN:V | | | | | | Novel | |
| | | | | | | | | | 49566 (human) | | | | | | | |
| AA271024 | 0 | −4.00 | −4.00 | −6.00 | −1.33 | 1.08 | −1.09 | 1.92 | small nuclear | AA271024 | | | | | Other | |
| | | | | | | | | | ribonucleoprot | | | | | | | |
| | | | | | | | | | ein D2 | | | | | | | |
| | | | | | | | | | polypeptide | | | | | | | |
| | | | | | | | | | (SNRPD2) | | | | | | | |
| Msa.31660.0 | 0 | −1.58 | 1.37 | 1.07 | 3.07 | 1.20 | 1.53 | 1.87 | CD53 antigen; | AA105582 | | LEUKOCYTE | | 3 50.5 cM | Cell Surface | Cd53 |
| | | | | | | | | | Cd53 | | | SURFACE | | | | |
| | | | | | | | | | | | | ANTIGEN | | | | |
| | | | | | | | | | | | | CD53 (CELL | | | | |
| | | | | | | | | | | | | SURFACE | | | | |
| | | | | | | | | | | | | GLYCOPROT | | | | |
| | | | | | | | | | | | | EIN CD53). | | | | |
| ET62056 | 0 | −3.22 | −1.76 | −2.32 | 1.45 | 1.29 | 1.33 | 1.86 | immunoglobuh | ET62056 | | | | | Extracellular | |
| | | | | | | | | | n rearranged | | | | | | Protein | |
| | | | | | | | | | kappa chain | | | | | | | |
| ab009287 | 0 | −2.33 | −3.50 | −7.00 | 0.00 | 1.14 | 0.00 | 1.86 | CD68 antigen; | AB009287 | | MACROSIALI | EXPRESSED | 11 39.0 cM | Cell Surface | Cd68 |
| | | | | | | | | | Cd68 | | | N | IN TISSUE | | | |
| | | | | | | | | | | | | PRECURSOR | MACROPHAG | | | |
| | | | | | | | | | | | | (CD68 | ES AND TO A | | | |
| | | | | | | | | | | | | ANTIGEN). | LESSER | | | |
| | | | | | | | | | | | | | EXTENT IN | | | |
| | | | | | | | | | | | | | DENDRITIC | | | |
| | | | | | | | | | | | | | CELLS. | | | |
| AA241085 | 0 | −4.00 | 1.17 | 1.08 | 1.42 | 2.08 | 1.67 | 1.83 | GENESEQN:Z | AA241085 | | | | | | |
| | | | | | | | | | 34468 Mouse | | | | | | | |
| | | | | | | | | | 15 kDa | | | | | | | |
| | | | | | | | | | selenoprotein | | | | | | | |
| AA020104 | 0 | −3.25 | −1.62 | −1.30 | 1.23 | 1.62 | 1.23 | 1.77 | glycosylation | AA020104 | | SULFATED 50 | LYMPH | 15 63.0 cM | | Glycam1 |
| | | | | | | | | | dependent cell | | | KDA | NODES. | | | |
| | | | | | | | | | adhesion | | | GLYCOPROT | | | | |
| | | | | | | | | | molecule 1; | | | EIN | | | | |
| | | | | | | | | | Glycam1 | | | PRECURSOR | | | | |
| | | | | | | | | | | | | (SGP50) | | | | |
| | | | | | | | | | | | | (ENDOTHELI | | | | |
| | | | | | | | | | | | | AL LIGAND | | | | |
| | | | | | | | | | | | | FOR L- | | | | |
| | | | | | | | | | | | | SELECTIN) | | | | |
| | | | | | | | | | | | | (GLYCOSYLA | | | | |
| | | | | | | | | | | | | TION- | | | | |
| | | | | | | | | | | | | DEPENDENT | | | | |
| | | | | | | | | | | | | CELL | | | | |
| | | | | | | | | | | | | ADHESION | | | | |
| | | | | | | | | | | | | MOLECULE | | | | |
| Msa.38664.0 | 0 | −2.13 | −1.06 | −1.89 | 4.88 | 1.12 | 1.06 | 1.76 | | AA144469 | | | | | EST; Unknown | |
| X94353 | 0 | −1.33 | 3.00 | 2.25 | 5.50 | 1.75 | 1.75 | 1.75 | cathelin-like | X94353 | | CATHELIN- | EXPRESSED | 9 61.0 cM | Metabolic | Cnlp |
| | | | | | | | | | protein; Cnlp | | | RELATED | IN TESTIS, | | | |
| | | | | | | | | | | | | ANTIMICROB | SPLEEN, | | | |
| | | | | | | | | | | | | IAL PEPTIDE | STOMACH, | | | |
| | | | | | | | | | | | | PRECURSOR | AND | | | |
| | | | | | | | | | | | | (CRAMP) | INTESTINE | | | |
| | | | | | | | | | | | | (CATHELIN- | VERY LOW | | | |
| | | | | | | | | | | | | LIKE | EXPRESSION | | | |
| | | | | | | | | | | | | PROTEIN) | FOUND IN | | | |
| | | | | | | | | | | | | (CLP). | HEART, | | | |
| | | | | | | | | | | | | | LUNG AND | | | |
| | | | | | | | | | | | | | SKELETAL | | | |
| | | | | | | | | | | | | | MUSCLE. NO | | | |
| | | | | | | | | | | | | | EXPRESSION | | | |
| | | | | | | | | | | | | | IN BRAIN, | | | |
| | | | | | | | | | | | | | KIDNEY OR | | | |
| | | | | | | | | | | | | | LIVER. | | | |
| ET62844 | 0 | −1.33 | 1.25 | −1.33 | 3.13 | 1.75 | 1.38 | 1.75 | paired-Ig-like | ET62844 | | | | 7 | Receptor | Pira10,Pira16 |
| | | | | | | | | | receptor | | | | | | | |
| | | | | | | | | | A10,paired-Ig- | | | | | | | |
| | | | | | | | | | like receptor | | | | | | | |
| | | | | | | | | | A6; | | | | | | | |
| | | | | | | | | | Pira10,Pira6 | | | | | | | |
| U06119 | 0 | −2.88 | −1.64 | −3.29 | 1.17 | 1.35 | 1.26 | 1 74 | cathepsin H; | U06119 | 3.4.22.16 | CATHEPSIN | WIDELY | 9 50.0 cM | Proteolytic | Ctsh |
| | | | | | | | | | Ctsh | | | II | EXPRESSED | | | |
| | | | | | | | | | | | | PRECURSOR | WITH | | | |
| | | | | | | | | | | | | (EC 3.4.22.16) | HIGHEST | | | |
| | | | | | | | | | | | | (CATHEPSIN | EXPRESSION | | | |
| | | | | | | | | | | | | B3) | FOUND IN | | | |
| | | | | | | | | | | | | (CATHEPSIN | NON- | | | |
| | | | | | | | | | | | | BA). | SKELETAL | | | |
| | | | | | | | | | | | | | TISSUES. | | | |
| | | | | | | | | | | | | | LOW LEVELS | | | |
| | | | | | | | | | | | | | FOUND IN | | | |
| | | | | | | | | | | | | | SKELETAL | | | |
| | | | | | | | | | | | | | TISSUE. | | | |
| M35153 | 0 | −1.75 | 1.29 | 1.29 | 3.71 | 1.71 | 2.00 | 1.71 | lamin B1; | M35153 | | LAMIN B1. | | 18 29.0 cM | ECM (Matrix | Lmnb1 |
| | | | | | | | | | Lmnb1 | | | | | | Prot) | |
| Msa.739.0 | 0 | −1.04 | 2.08 | 1.17 | 5.63 | 1.79 | 1.33 | 1.71 | haptoglobin; | M96827 | | | | 8 55.0 cM | Extracellular | Hp |
| | | | | | | | | | Hp | | | | | | Protein | |
| AA445408 | 0 | −5.00 | −2.50 | −10.00 | −1.43 | −1.43 | 1.20 | 1.70 | H3 histone, | AA445408 | | | | | Other | H3f3b |
| | | | | | | | | | family 3B; | | | | | | | |
| | | | | | | | | | H3f3b | | | | | | | |
| Msa.7498.0 | 0 | −1.50 | 1.67 | 1.33 | 3.67 | 0.00 | 1.33 | 1.67 | growth arrest | AA138777 | | GROWTH | | | Regulatory | Gadd45g |
| | | | | | | | | | and DNA- | | | ARREST AND | | | | |
| | | | | | | | | | damage- | | | DNA- | | | | |
| | | | | | | | | | inducible, | | | DAMAGE- | | | | |
| | | | | | | | | | gamma; | INDUCIBLE | | | | | | |
| | | | | | | | | | Gadd45g | PROTEIN | | | | | | |
| | | | | | | | | | | | | GADD45 | | | | |
| | | | | | | | | | | | | GAMMA | | | | |
| | | | | | | | | | | | | (CYTOKINE | | | | |
| | | | | | | | | | | | | RESPONSIVE | | | | |
| Msa.1903.0 | 0 | −3.63 | −2.07 | −4.83 | 1.03 | −1.61 | −1.38 | 1.66 | histoconspatibil | U35323 | | CLASS II | 17 18.56 | □ | H2-DMa,H2- | |
| | | | | | | | | | ity 2, class II, | | | HISTOCOMP | cM,17 l8.~ | | | |
| | | | | | | | | | locus | | | ATIBILITY | cM,17 l8.~ | | | |
| | | | | | | | | | DMa,histocom | | | ANTIGEN, M | cM,17 I8.~ | | | |
| | | | | | | | | | patibility 2, | | | ALPHA | cM | | | |
| | | | | | | | | | class II, locus | | | CHAIN | | | | |
| | | | | | | | | | Mb1,histocom | | | PRECURSOR. | | | | |
| | | | | | | | | | patibilily 2, | | | ,CLASS II | | | | |
| | | | | | | | | | class II, locus | | | HISTOCOMP | | | | |
| | | | | | | | | | Mb2,proteoso | | | ATIBILITY | | | | |
| | | | | | | | | | me(prosome, | | | ANTIGEN, M | | | | |
| | | | | | | | | | macropain) | | | BETA 1 | | | | |
| | | | | | | | | | subunit, beta | | | CHAIN | | | | |
| | | | | | | | | | type 9 (large | | | PRECURSOR | | | | |
| | | | | | | | | | multifunctional | | | (H2-M BETA | | | | |
| | | | | | | | | | prolease 2); 1 | | | | | | | |
| | | | | | | | | | DMa,H2- | CHAIN).,PRO | | | | | | |
| | | | | | | | | | DMb1,H2- | TEASOME | | | | | | |
| | | | | | | | | | DMb2,Psmb9 | CHAIN 7 | | | | | | |
| | | | | | | | | | | | | | PRECURSOR | | | |
| | | | | | | | | | | | | | (EC 3.4.99.46) | | | |
| | | | | | | | | | | | | | (MACROPAIN | | | |
| | | | | | | | | | | | | | CHAIN 7) | | | |
| | | | | | | | | | | | | | (MULTICATA | | | |
| | | | | | | | | | | | | | LYTIC | | | |
| | | | | | | | | | | | | | ENDOPEPTID | | | |
| | | | | | | | | | | | | | ASE | | | |
| U96689 | 0 | −1.06 | 1.59 | 1.29 | 3.18 | 1.94 | 1.65 | 1.65 | paired-Ig-like | U96689 | | | | 7 1.0 cM | Receptor | Pirb |
| | | | | | | | | | receptor B; | | | | | | | |
| | | | | | | | | | Pirb | | | | | | | |
| V01527 | 0 | −3.55 | −1.95 | −4.33 | 0.00 | −1.30 | −1.11 | 1.62 | histocompatibil | V01527 | | H-2 CLASS II | | 17 18.64 cM | Cell Surface | II2-Ab1 |
| | | | | | | | | | ity 2, class II | | | HISTOCOMP | | | | |
| | | | | | | | | | antigen A, beta | | | ATIBILITY | | | | |
| | | | | | | | | | 1; H2-Ab1 | | | ANTIGEN, A- | | | | |
| | | | | | | | | | | | | D BETA | | | | |
| | | | | | | | | | | | CHAIN | | | | | |
| AA285691 | 0 | −3.29 | −1.55 | −2.32 | 1.37 | 1.37 | 1.01 | 1.61 | cytohesin | AA285691 | | | | | Cell Surface | |
| | | | | | | | | | binding protein | | | | | | Protein | |
| | | | | | | | | | (Cbp) | | | | | | | |
| X51829 | 0 | −5.00 | −2.50 | −3.33 | −1.25 | −1.25 | −1.11 | 1.60 | myeloid | X51829 | | MYELOID | | | Other | Myd116 |
| | | | | | | | | | differentiation | | | DIFFERENTI | | | | |
| | | | | | | | | | primary | | | ATION | | | | |
| | | | | | | | | | response gene | | | PRIMARY | | | | |
| | | | | | | | | | 116; Myd116 | | | RESPONSE | | | | |
| | | | | | | | | | | | | PROTEIN | | | | |
| | | | | | | | | | | | | MYD116. | | | | |
| AA185060 | 0 | −3.50 | −2.33 | −2.33 | 0.00 | 1.29 | 0.00 | 1.57 | GENESEQN:Z | AA185060 | | | | | Unknown | |
| | | | | | | | | | 52941 Human | | | | | | | |
| | | | | | | | | | prostate tumor | | | | | | | |
| | | | | | | | | | cDNA library | | | | | | | |
| | | | | | | | | | derived EST | | | | | | | |
| | | | | | | | | | fragment #84 | | | | | | | |
| U59488 | 0 | −1.75 | −2.33 | −3.50 | 1.43 | −1.17 | 0 00 | 1.57 | neutrophil | U59488 | | NEUTROPHIL | | 15 47.2 | Intracellular | Ncf4 |
| | | | | | | | | | cytosolic factor | | | CYTOSOL | | | | |
| | | | | | | | | | 4, Ncf4 | | | FACTOR 4 | | | | |
| | | | | | | | | | | | | (NCF-4) | | | | |
| | | | | | | | | | | | | (NEUTROPHI | | | | |
| | | | | | | | | | | | | L NADPH | | | | |
| | | | | | | | | | | | | OXIDASE | | | | |
| | | | | | | | | | | | | FACTOR 4) | | | | |
| | | | | | | | | | | | | (P40-PHOX) | | | | |
| | | | | | | | | | | | | (P40PHOX). | | | | |
| L37297 | 0 | −2.00 | 3.00 | 2.00 | 20.75 | 2.50 | 2.00 | 1.50 | neutrophilic | L37297 | | | | | Intracellular | Ngp |
| | | | | | | | | | granule | | | | | | Protein | |
| | | | | | | | | | protein; Ngp | | | | | | | |
| L37297 | 0 | −2.00 | 3.00 | 2.00 | 20.75 | 2.50 | 2.00 | 1.50 | neutrophilic | L37297 | | | | | Intracellular | Ngp |
| | | | | | | | | | granule | | | | | | Protein | |
| | | | | | | | | | protein; Ngp | | | | | | | |
| D73368 | 0 | −4.00 | −3.00 | −6.00 | −1.20 | 1.08 | 0.00 | 1.50 | enhancer of | D73368 | | DEAD-BOX | DEVELOPME | | Other | Erh |
| | | | | | | | | | rudimentary | | | PROTEIN 3 | NTALLY | | | |
| | | | | | | | | | homolog | | | (DEAD-BOX | REGULATED. | | | |
| | | | | | | | | | (Drosophila); | | | RNA | | | | |
| | | | | | | | | | Erh | | | HELICASE | | | | |
| | | | | | | | | | | | | DEAD3) | | | | |
| | | | | | | | | | | | | (MDEAD3) | | | | |
| | | | | | | | | | | | | (EMBRYONIC | | | | |
| | | | | | | | | | | | | EllA | | | | |
| | | | | | | | | | | | | HELICASE) | | | | |
| | | | | | | | | | | | | (D1PAS1 | | | | |
| | | | | | | | | | | | | RELATED | | | | |
| K01923 | 0 | −3.75 | −1.54 | −2.92 | −1.14 | 1.04 | 1.03 | 1.50 | histocompatibil | K01923 | | H-2 CLASS II | | 17 18.65 cM | Cell Surface | H2-Aa |
| | | | | | | | | | ity 2, class II | | | HISTOCOMP | | | Protein | |
| | | | | | | | | | antigen A, | | | ATIBILITY | | | | |
| | | | | | | | | | alpha; H2-Aa | | | ANTIGEN, A- | | | | |
| | | | | | | | | | | | | K ALPHA | | | | |
| | | | | | | | | | | | | CHAIN | | | | |
| Msa.1700.0 | 0 | −1.37 | 1.36 | −1.29 | 3.14 | 1.32 | 1.09 | 1.45 | phospholipase | U34277 | 3.1.1.47 | PLATELET- | PLASMA. | | Cytokine | Pla2g7 |
| | | | | | | | | | A2 group VII | | | ACTIVATING | | | | |
| | | | | | | | | | (platelet- | | | FACTOR | | | | |
| | | | | | | | | | factor | | | ACETYLHYD | | | | |
| | | | | | | | | | acetylhydrolase | PRECURSOR | | | | | | |
| | | | | | | | | | ,plasma); | (EC 3.1.1.47) | | | | | | |
| | | | | | | | | | Pla2g7 | (PAF | | | | | | |
| | | | | | | | | | | | | ACETYLHYD | | | | |
| | | | | | | | | | | | | ROLASE) | | | | |
| | | | | | | | | | | | | (PAF 2- | | | | |
| | | | | | | | | | | | | ACYLHYDR( | | | | |
| | | | | | | | | | | | | LASE) (LDL- | | | | |
| | | | | | | | | | | | | ASSOCIATED | | | | |
| | | | | | | | | | | | | PHOSPHOLIP | | | | |
| | | | | | | | | | | | | ASE A2) (LDL- | | | | |
| | | | | | | | | | | | | PLA(2)) (2- | | | | |
| | | | | | | | | | | | | ACETYL-1- | | | | |
| | | | | | | | | | | | | ALKYLGLYC | | | | |
| | | | | | | | | | | | | EROPHOSPH | | | | |
| | | | | | | | | | | | | OCHOLINE | | | | |
| | | | | | | | | | | | | ESTERASE) | | | | |
| | | | | | | | | | | | | (1-ALKYL-2- | | | | |
| | | | | | | | | | | | | ACETYLGLY | | | | |
| | | | | | | | | | | | | CEROPHOSP | | | | |
| | | | | | | | | | | | | HOCHOLINE | | | | |
| C76739 | 0 | −1.29 | 1.33 | 1.33 | 3.44 | 2.11 | 1.78 | 1.44 | macrophage C- | C76739 | | | | 6 56.5 cM | Cell Surface | Mpc1 |
| | | | | | | | | | type lectin; | | | | | | Protein | |
| | | | | | | | | | Mpc1 | | | | | | | |
| U29947 | 0 | −2.29 | −1.78 | −4.00 | 1.44 | 1.19 | 1.13 | 1.44 | mannosidase 2, | U29947 | | LYSOSOMAL | | 8 37.0 cM | Regulatory | Man2b1 |
| | | | | | | | | | alpha B1; | | | ALPHA- | | | | |
| | | | | | | | | | Man2b1 | | | MANNOSIDA | | | | |
| | | | | | | | | | | | | SE | | | | |
| | | | | | | | | | | | | PRECURSOR | | | | |
| | | | | | | | | | | | | (EC 3.2.1.24) | | | | |
| | | | | | | | | | | | | (MANNOSID | | | | |
| | | | | | | | | | | | | ASE, ALPHA | | | | |
| | | | | | | | | | | | | B) | | | | |
| | | | | | | | | | | | | (LYSOSOMA | | | | |
| | | | | | | | | | | | | L ACID | | | | |
| AA008321 | 0 | −1.75 | −1.75 | −3.50 | 1.43 | 1.86 | 0.00 | 1.43 | proteasome | AA08321 | 3.4.99.46 | PROTEASOM | | | Proteolytic | Psma4 |
| | | | | | | | | | (prosome, | | | E | | | | |
| | | | | | | | | | macropain) | | | COMPONENT | | | | |
| | | | | | | | | | subunit, alpha | | | C9 (EC | | | | |
| | | | | | | | | | type 4; Psma4 | | | 3.4.99.46) | | | | |
| | | | | | | | | | | | | (MACROPAIN | | | | |
| | | | | | | | | | | | | SUBUNIT C9) | | | | |
| | | | | | | | | | | | | (MULTICATA | | | | |
| | | | | | | | | | | | | LYTIC | | | | |
| | | | | | | | | | | | | ENDOPEPTID | | | | |
| | | | | | | | | | | | | ASE | | | | |
| T25659 | 0 | −3.50 | −1.40 | −2.33 | 1.14 | 1.14 | 0.00 | 1.43 | heterogeneous | T25659 | | HETEROGEN | | | EST; Unknown | Hnrpa2b1 |
| | | | | | | | | | nuclear | | | EOUS | | | | |
| | | | | | | | | | ribonucleoprot | | | NUCLEAR | | | | |
| | | | | | | | | | ein A2/B1; | | | RIBONUCLE | | | | |
| | | | | | | | | | Hnrpa2b1 | | | OPROTEINS | | | | |
| | | | | | | | | | | | | A2/B1 | | | | |
| | | | | | | | | | | | | (HNRNP A2 | | | | |
| AA638408 | 0 | −3.40 | −2.83 | −4.25 | −1.42 | −1.31 | −1.13 | 1.41 | arginine N- | AA638408 | | | | | Signal | Mrmt1 |
| | | | | | | | | | methyltransfera | | | | | | Transduction | |
| | | | | | | | | | se 1; Mrmt1 | | | | | | | |
| AA408475 | 0 | −3.33 | −2.50 | −2.50 | −1.11 | 1.50 | 1.30 | 1.40 | ribosomal | AA408475 | | 60S | | 7 25.0 cM | Intracellular | Rp113a |
| | | | | | | | | | protein L13a; | | | RIBOSOMAL | | | Protein | |
| | | | | | | | | | Rp113a | | | PROTEIN | | | | |
| | | | | | | | | | | | | L13A | | | | |
| | | | | | | | | | | | | (TRANSPLAN | | | | |
| | | | | | | | | | | | | TATION | | | | |
| | | | | | | | | | | | | ANTIGEN | | | | |
| | | | | | | | | | | | | P198) (TUM- | | | | |
| | | | | | | | | | | | | P198 | | | | |
| M59378 | 0 | −3.25 | −1.86 | −3.25 | −1.18 | 1.08 | −1.30 | 1.38 | tumor necrosis | M59378 | | TUMOR | | 4 75.5 cM | Receptor | Tnfrsf1b |
| | | | | | | | | | factor receptor | | | NECROSIS | | | | |
| | | | | | | | | | superfamily, | | | FACTOR | | | | |
| | | | | | | | | | member 1b; | | | RECEPTOR 2 | | | | |
| | | | | | | | | | Tnfrsf1b | | | PRECURSOR | | | | |
| | | | | | | | | | | | | (TNF-R2) | | | | |
| | | | | | | | | | | | | (P75). | | | | |
| AA189914 | 0 | −1.26 | 2.08 | 2.83 | 2.79 | 3.92 | 2 38 | 1.38 | cytidine | AA189914 | | | | 6 74.0 cM | | Cmas |
| | | | | | | | | | monophospho- | | | | | | | |
| | | | | | | | | | N- | | | | | | | |
| | | | | | | | | | acetylneuramin | | | | | | | |
| | | | | | | | | | ic acid | | | | | | | |
| | | | | | | | | | synthetase; | | | | | | | |
| | | | | | | | | | Cmas | | | | | | | |
| AA261402 | 0 | −2.78 | −1.92 | −3.57 | 1.04 | 1.24 | −1.14 | 1.36 | EST; Unknown | AA261402 | | | | | EST; Unknown | Unknown |
| AA174982 | 0 | −2.37 | −1.76 | −3.10 | 1.17 | 1.11 | −1.17 | 1.36 | coronin, actin | AA174982 | | CORONIN- | | 7 62.5 cM | Intracellular | Corola |
| | | | | | | | | | binding protein | | | LIKE | | | Protein | |
| | | | | | | | | | 1A; Corola | | | PROTEIN P57 | | | | |
| | | | | | | | | | | | | (CORONIN | | | | |
| | | | | | | | | | | | | 1A) | | | | |
| AA182228 | 0 | −2.56 | −3.29 | −7.67 | −1.15 | −1.21 | −1.28 | 1.35 | EST; Unknown | AA182228 | | | | | EST; Unknown | Unknown |
| AF032466 | 0 | −1.50 | 2.33 | 1.67 | 6.00 | 2.33 | 1.33 | 1.33 | arginase type | AF032466 | 3.5.3.1 | ARGINASE II | | | | Arg2 |
| | | | | | | | | | II; Arg2 | | | PRECURSOR | | | | |
| | | | | | | | | | | | | (EC 3.5 3.1) | | | | |
| | | | | | | | | | | | | (NON- | | | | |
| | | | | | | | | | | | | HEPATIC | | | | |
| | | | | | | | | | | | | ARGINASE) | | | | |
| | | | | | | | | | | | | (KIDNEY- | | | | |
| | | | | | | | | | | | | TYPE | | | | |
| AA152590 | 0 | −3.00 | −2.25 | −4.50 | −1.29 | 1.44 | 1.11 | 1.33 | eukaryotic | AA152590 | | | | 11 39.0 cM | Translation | Eif4a1 |
| | | | | | | | | | translation | | | | | | Factor | |
| | | | | | | | | | Initiation factor | | | | | | | |
| | | | | | | | | | 4A1; Eif4a1 | | | | | | | |
| AA273932 | 0 | −3.57 | −2.08 | −2.78 | −1.19 | 1.08 | −1.09 | 1.32 | aldo-keto | AA273932 | | | | | Other | LOC56043 |
| | | | | | | | | | reductase; | | | | | | | |
| | | | | | | | | | LOC56043 | | | | | | | |
| AA189758 | 0 | −3.25 | −1.86 | −3.25 | −1.18 | 1.08 | 1.15 | 1.31 | Wbscr5 gene | AA189758 | | | | | Regulatory | Ebser5 |
| | | | | | | | | | product; | | | | | | | |
| | | | | | | | | | Wbscr5 | | | | | | | |
| Msa.4530.0 | 0 | −2.88 | −1.92 | −3.83 | 0.00 | −1.15 | −1.15 | 1.30 | EST; region of | AA106931 | | | | | EST; Unknown | |
| | | | | | | | | | homolgy to | | | | | | | |
| | | | | | | | | | GENESEQN:Z | | | | | | | |
| | | | | | | | | | 77537 Human | | | | | | | |
| | | | | | | | | | ovarian tumor | | | | | | | |
| | | | | | | | | | eDNA library | | | | | | | |
| | | | | | | | | | derived EST | | | | | | | |
| | | | | | | | | | fragment 88 | | | | | | | |
| K01925 | 0 | −3.84 | −1.66 | −3.31 | −1A6 | −1.10 | −1.10 | 1.30 | histocompatibil | K01925 | | H-2 CLASS II | | 17 18.65 cM | Hemostasis | H2-Aa |
| | | | | | | | | | ity 2, class II | | | HISTOCOMP | | | | |
| | | | | | | | | | antigen A, | | | ATIBILITY | | | | |
| | | | | | | | | | alpha; H2-Aa | | | ANTIGEN, A- | | | | |
| | | | | | | | | | | | | K ALPHA | | | | |
| | | | | | | | | | | | | CHAIN | | | | |
| D10911 | 0 | −1.27 | 1.93 | 1.57 | 4.50 | 2.00 | 1.64 | 1.29 | a disintegrin | D10911 | 3.4.24.- | ADAM 8 | MACROPHAG | 7 F3-F5 | Proteolytic | Adam8 |
| | | | | | | | | | and | | | PRECURSOR | ES. | | | |
| | | | | | | | | | metalloproteas | | | (EC 3.4.24.0_ | | | | |
| | | | | | | | | | c domain | | | (A | | | | |
| | | | | | | | | | (ADAM) 8; | | | DISINTEGRIN | | | | |
| | | | | | | | | | Adam8 | | | AND | | | | |
| | | | | | | | | | | | | METALLOPR | | | | |
| | | | | | | | | | | | | OTEINASE | | | | |
| | | | | | | | | | | | | DOMAIN 8) | | | | |
| | | | | | | | | | | | | (CELL | | | | |
| | | | | | | | | | | | | SURFACE | | | | |
| | | | | | | | | | | | | ANTIGEN | | | | |
| | | | | | | | | | | | | MS2) | | | | |
| | | | | | | | | | | | | (MACROPHA | | | | |
| | | | | | | | | | | | | GE | | | | |
| | | | | | | | | | | | | CYSTEINE- | | | | |
| | | | | | | | | | | | | RICH | | | | |
| M59821 | ###### | −3.15 | −5.13 | −5.86 | −1.58 | −1.78 | −1.58 | 1.27 | immediate | M59821 | | T- | | | Intracellular | Ier2 |
| | | | | | | | | | early response | | | LYMPHOCYT | | | | |
| | | | | | | | | | 2; Ier2 | | | E | | | | |
| | | | | | | | | | | | | ACTIVATED | | | | |
| | | | | | | | | | | | | PROTEIN | | | | |
| | | | | | | | | | | | | (CYCLOHEXI | | | | |
| | | | | | | | | | | | | MIDE- | | | | |
| | | | | | | | | | | | | INDUCED) | | | | |
| | | | | | | | | | | | | (CHXI) | | | | |
| | | | | | | | | | | | | (IMMEDIATE | | | | |
| | | | | | | | | | | | | EARLY | | | | |
| AA575696 | 0 | −3.00 | −3.00 | −4.00 | 1.33 | 1.17 | 1.08 | 1.25 | sorting nexin 1; | AA575696 | | | | | Regulatory | Snx1 |
| | | | | | | | | | Snx1 | | | | | | | |
| AA575696 | 0 | −3 00 | −3.00 | −4.00 | 1.33 | 1.17 | 1.08 | 1.25 | sorting nexin 1; | AA575696 | | | | | Regulatory | Snx1 |
| | | | | | | | | | Snx1 | | | | | | | |
| AA172851 | 0 | −1.26 | 2.24 | 1.66 | 5.66 | 2.17 | 1.41 | 1.21 | EST; Unknown | AA172851 | | | | | EST; Unknown | Unknown |
| AA285635 | 0 | −3.00 | −2.40 | −6.00 | −1.33 | 1.17 | −1.71 | 1.17 | ectoplacental | AA285635 | | | | | EST; Unknown | Epcs21- |
| | | | | | | | | | cone, invasive | | | | | | | |
| | | | | | | | | | trophoblast | | | | | | | |
| | | | | | | | | | giant cells, | | | | | | | |
| | | | | | | | | | extraembryonic | | | | | | | |
| | | | | | | | | | ectoderm and | | | | | | | |
| | | | | | | | | | chorion | | | | | | | |
| | | | | | | | | | sequence 21; | | | | | | | |
| | | | | | | | | | Epcs21- | | | | | | | |
| | | | | | | | | | pending | | | | | | | |
| AA254740 | 0 | −3.75 | −1.67 | −2.50 | −1.15 | 1.13 | 0.00 | 1.13 | Nop10p | AA254740 | | | | | Other | |
| | | | | | | | | | (human) | | | | | | | |
| Msa.928.0 | 0 | −1.60 | 1.38 | 0.00 | 3.50 | 1.38 | 1.13 | 1.13 | myristoylated | M60474 | | MYRISTOYL | BRAIN, | 10 22.0 cM | Structural | Nacs |
| | | | | | | | | | alanine rich | | | ATED | SPLEEN, | | Protein | |
| | | | | | | | | | protein kinase | | | ALANINE- | LESS IN | | | |
| | | | | | | | | | C substrate | | | RICH C- | KIDNEY AND | | | |
| | | | | | | | | | macs | | | KINASE | HEART, AND | | | |
| | | | | | | | | | | | | SUBSTRATE | VERY LOW | | | |
| | | | | | | | | | | | | (MARCKS). | LEVELS IN | | | |
| AA020128 | 0 | −4.50 | −3.00 | −3.00 | 1.33 | 1.44 | 1.11 | 1.11 | | AA020128 | | | | | | |
| U91848 | 0 | −5.00 | −2.22 | −4.00 | −1.43 | −1.33 | 0.00 | 1.10 | clathrin, light | U91848 | | CLATHRIN | | | Structural | C1ta |
| | | | | | | | | | polypeptide | | | LIGHT CHAIN | | | Protein | |
| | | | | | | | | | (Lca); C1ta | A. | | | | | | |
| U16985 | 0 | −3 12 | −3.00 | −4.26 | −1.19 | −1.09 | −1.31 | 1.05 | lymphotoxin B; | U16985 | | LYMPHOTOX | | 17 19.06 cM | Cytokine | Ltb |
| | | | | | | | | | Ltb | | | IN-BETA (LT- | | | | |
| | | | | | | | | | | | | BETA) | | | | |
| | | | | | | | | | | | | (TUMOR | | | | |
| | | | | | | | | | | | | NECROSIS | | | | |
| | | | | | | | | | | | | FACTOR C). | | | | |
| Z27231 | 0 | −1.17 | 2.00 | 0.00 | 5.86 | 1.71 | 1.29 | 0.00 | matrix | Z27231 | 3.4.24.35 | 92 KDA TYPE | | 2 96.0 cM | Metabolic | Mmp9 |
| | | | | | | | | | metalloprotein | | | IV | | | | |
| | | | | | | | | | ase 9; Mmp9 | | | COLLAGENA | | | | |
| | | | | | | | | | | | | SE | | | | |
| | | | | | | | | | | | | PRECURSOR | | | | |
| | | | | | | | | | | | | (EC 3 4.24.35) | | | | |
| | | | | | | | | | | | | (92 KDA | | | | |
| | | | | | | | | | | | | GELATINASE | | | | |
| | | | | | | | | | | | | ) (MATRIX | | | | |
| | | | | | | | | | | | | METALLOPR | | | | |
| | | | | | | | | | | | | OTEINASE-9) | | | | |
| | | | | | | | | | | | | (MMP-9) | | | | |
| | | | | | | | | | | | | (GELATINAS | | | | |
| D17630 | 0 | −1.20 | 1.83 | 0.00 | 4.50 | 1.83 | 1.33 | −1.20 | chemokine(C- | D17630 | | HIGH | | 1 40.0 cM | | Cmkar2 |
| | | | | | | | | | X-C) receptor | | | AFFINITY | | | | |
| | | | | | | | | | 2; Cmkar2 | | | INTERLEUKI | | | | |
| | | | | | | | | | | | | N-8 | | | | |
| | | | | | | | | | | | | RECEPTOR B | | | | |
| | | | | | | | | | | | | (IL-8R B) | | | | |
| | | | | | | | | | | | | (CXCR-2) | | | | |
| | | | | | | | | | | | | (GRO/MGSA | | | | |
| AA407584 | 0 | −4.00 | −2.00 | −3.00 | −2.00 | −1.09 | −1.33 | −1.20 | DNA segment, | AA407584 | | | | 7 69.0 cM, 7 | | D7Wsu30e |
| | | | | | | | | | Chr 7, Wayne | | | | | | | |
| | | | | | | | | | State | | | | | | | |
| | | | | | | | | | University 30, | | | | | | | |
| | | | | | | | | | expressed,nucl | | | | | | | |
| | | | | | | | | | cosome | | | | | | | |
| | | | | | | | | | assembly | | | | | | | |
| | | | | | | | | | protein 1 | | | | | | | |
| | | | | | | | | | 4; | | | | | | | |
| | | | | | | | | | D7Wsu30e,Na | | | | | | | |
| | | | | | | | | | p114 | | | | | | | |
| AA529094 | 0 | −3.33 | −2.50 | −3.33 | −1 43 | −1.25 | −1.11 | −1.25 | | AA529094 | | | | | | |
| X93037 | 0 | −1.50 | −1.50 | −1.50 | 3.33 | 0.00 | −1.50 | −1.50 | extracellular | X93037 | | WDNM1 | | | | Expi |
| | | | | | | | | | proteinase | | | PROTEIN | | | | |
| | | | | | | | | | inhibitor; Expi | | | PRECURSOR | | | | |
| M22326-2 | 0 | −6.00 | −4.40 | −11.00 | −6.00 | −2.64 | −3.88 | −1.69 | early growth | M22326 | | EARLY | | 18 16.0 cM | Intracellular | Egr1 |
| | | | | | | | | | response 1; | | | GROWTH | | | | |
| | | | | | | | | | Egr1 | RESPONSE | | | | | | |
| | | | | | | | | | | | | PROTEIN 1 | | | | |
| | | | | | | | | | | | | (EGR-1) | | | | |
| | | | | | | | | | | | | (KROX-24 | | | | |
| | | | | | | | | | | | | PROTEIN) | | | | |

TBS = total body score

TABLE 5
CIA-001,011,012pawsU-FC
Raw data from GEDS Fold Change, Genes were originally filtered for Present/Absent using frequency data

| Note here | con- | pre- | | | Score | | | | | | | | | | | | | |
|---|---|---|---|---|---|---|---|---|---|---|---|---|---|---|---|---|---|---|
| Note here | trol | arth | Score 1 | Std | 2 | Std | Score 3 | Std | Score 4 | Std | | | | | | | Kinase or | |
| Score( )* | C | P | 1 | Err | 2 | Err | 2 | Err | 3 | Err | 4 | Err | | | | | | Phos- |
| Systematic | Raw | Raw | Raw | Raw | Raw | Raw | Raw | Raw | Raw | Raw | Common | Genbank | description | function | Map | Keywords | phatase | |
| D37801 | 1.00 | −1.13 | −2 72 | 0.68 | −1.99 | 0.15 | −2.79 | 0.82 | −2.51 | 0.14 | protein tyrosine | D37801 | protein tyrosine | may be involved in the | | | Phos- | |
| | | | | | | | | | | | phosphatase, | | phosphatase, non- | regulation of growth | | | phatase | |
| | | | | | | | | | | | non-receptor | | receptor type 21 | and differentiation of | | | | |
| | | | | | | | | | | | type 21; | | (ec 3.1.3.48) | liver cells. | | | | |
| | | | | | | | | | | | Ptpn 2) | | (protein- | | | | | |
| | | | | | | | | | | | | | tyrosine | | | | | |
| | | | | | | | | | | | | | phosphatase ptp- | | | | | |
| | | | | | | | | | | | | | r110) | | | | | |
| AA204199 | 1.00 | 2.86 | −3.91 | 2.24 | −0.03 | 1.37 | −2.26 | 0.12 | −2.68 | 0.30 | protein tyrosine | AA204199 | | northern blot analysis | | Signal | Phos- | |
| | | | | | | | | | | | phosphatase | | | revealed that pr1-2 is | | Transduction | phatase | |
| | | | | | | | | | | | 4a3, Ptp4a3 | | | preferentially | | | | |
| | | | | | | | | | | | | | | expressed in skeletal | | | | |
| | | | | | | | | | | | | | | muscle, while pr1-3 is | | | | |
| | | | | | | | | | | | | | | preferentially | | | | |
| | | | | | | | | | | | | | | expressed in both | | | | |
| | | | | | | | | | | | | | | skeletal muscle and | | | | |
| | | | | | | | | | | | | | | heart, although both | | | | |
| | | | | | | | | | | | | | | pr1-2 and pr1-3 are | | | | |
| | | | | | | | | | | | | | | expressed at lower | | | | |
| | | | | | | | | | | | | | | levels in other tissues | | | | |
| U28244 | 1.00 | −1.70 | −1.41 | 1.43 | −0.79 | 1.88 | −6.34 | 2.53 | −6.56 | 1.87 | phospholipase | U28244 | phospholipase a2, | pa2 catalyzes the | 4 68.0 cM | | Phos- | |
| | | | | | | | | | | | A2, group IIA | | membrane | calcium-dependent | | | phatase | |
| | | | | | | | | | | | (platelets, | | associated pre- | hydrolysis of the 2- | | | | |
| | | | | | | | | | | | synovial fluid); | | cursor | acyl groups in 3-sn- | | | | |
| | | | | | | | | | | | Pla2g2a | | (ec 3.1 1.4) | phosphoglycerides. | | | | |
| | | | | | | | | | | | | | (phosphatidylcho- | | | | | |
| | | | | | | | | | | | | | line | | | | | |
| | | | | | | | | | | | | | 2-acylhydrolase) | | | | | |
| | | | | | | | | | | | | | (group ii | | | | | |
| | | | | | | | | | | | | | phospholipase a2) | | | | | |
| | | | | | | | | | | | | | (enhancing factor) | | | | | |
| | | | | | | | | | | | | | (ef) | | | | | |
| Mta.30443.0 | 1.00 | 1.21 | 2.19 | 0.78 | 2.28 | 0.60 | 9.18 | 1.92 | 11.29 | 3.33 | RAS-related C3 | AA097231 | ras-related c3 | function: seems to be | | Intracellular | Kinase | |
| | | | | | | | | | | | botulinum | | botulinum toxin | involved in the | | Protein | | |
| | | | | | | | | | | | substrate | | substrate 2 (p21- | regulation of the | | | | |
| | | | | | | | | | | | 2; Rac2 | | rac2) | nadph oxidase. | | | | |
| | | | | | | | | | | | | | (en-7 protein). | subcellular location; | | | | |
| | | | | | | | | | | | | | | cytoplasmic; | | | | |
| | | | | | | | | | | | | | | membrane-associated | | | | |
| | | | | | | | | | | | | | | when activated. tissue | | | | |
| | | | | | | | | | | | | | | specificity: | | | | |
| | | | | | | | | | | | | | | hematopoietic | | | | |
| | | | | | | | | | | | | | | specific. similarity; | | | | |
| | | | | | | | | | | | | | | below to the small | | | | |
| J03023 | 1.00 | −1.22 | 0.45 | 0.71 | 0.96 | 2.17 | 3.77 | 0.26 | 4.24 | 0.44 | hemopoietic cell | J03023 | tyrosine-protein | may serve as part of a | 2 86.0 cM | Hemostasis | Kinase | |
| | | | | | | | | | | | kinase; Hck | | kinase hck (ec | signaling pathway | | | | |
| | | | | | | | | | | | | | 2.7.1.112) | coupling the fc | | | | |
| | | | | | | | | | | | | | (p56-hck | receptor to the | | | | |
| | | | | | | | | | | | | | and p60-hck) | activation of the | | | | |
| | | | | | | | | | | | | | (hemopoietic cell | respiratory burst. may | | | | |
| | | | | | | | | | | | | | kinase (b- | also contribute to | | | | |
| | | | | | | | | | | | | | cell/myeloid | neutrophil migration | | | | |
| | | | | | | | | | | | | | kinase) | any may regulate the | | | | |
| | | | | | | | | | | | | | (bmk). | degranulation process | | | | |
| | | | | | | | | | | | | | | of neutrophils | | | | |
| X06368 | 1.00 | −2.02 | −2.80 | 1.36 | −0.30 | 1.81 | 1.50 | 0.23 | 1.45 | 0.19 | colony | X06368 | macrophage colony | this protein is the | 18 | Cytokine | Kinase | |
| | | | | | | | | | | | stimulating | | stimulating | receptor for csf-1, it is | 30.0 cM | | | |
| | | | | | | | | | | | factor 1 | | factor i | a protein tyrosine- | | | | |
| | | | | | | | | | | | receptor; | | receptor | kinase transmembrane | | | | |
| | | | | | | | | | | | Csf1r | | | precursor | receptor. | | | |
| | | | | | | | | | | | | | (csf-1-r) (ec | | | | | |
| | | | | | | | | | | | | | 2.7.1.112)(fms | | | | | |
| | | | | | | | | | | | | | proto- | | | | | |
| | | | | | | | | | | | | | oncogene)(c- | | | | | |
| | | | | | | | | | | | | | fms) | | | | | |
| Msa.1709.0 | 1.00 | −1.81 | −3.77 | 0.76 | −1.37 | 2.51 | −1.58 | 0.33 | 0.10 | 0.93 | elastin; Eln | U08210 | elastin precursor | major structural | 5 75.0 cM | | Kinase | |
| | | | | | | | | | | | | | (tropoelastin). | protein of tissues such | | | | |
| | | | | | | | | | | | | | | as aorta and nuchal | | | | |
| | | | | | | | | | | | | | | ligament, which must | | | | |
| | | | | | | | | | | | | | | expand rapidly and | | | | |
| | | | | | | | | | | | | | | recover completely | | | | |
| Msa 6386.0 | 1.00 | 1.42 | −3.46 | 2 04 | −1.35 | 0.22 | −2.64 | 0.72 | −3.21 | 0.57 | mitogen activated | W13523 | | | 17 A3-B | | Kinase | |
| | | | | | | | | | | | protein kinase 13; | | | | | | | |
| | | | | | | | | | | | Mapk13 | | | | | | | |
| Msa.1160.0 | 1.00 | 1.00 | 17.56 | 13.65 | 12.86 | 11.86 | 106.21 | 7.39 | 107.32 | 28.80 | serum amyloid | X03505 | serum amylois | member of | 7 23.5 | Extracellular | | |
| | | | | | | | | | | | A3; Saa3 | | a-3 protein | “”a family | cM | Protein | | |
| | | | | | | | | | | | | | precursor. | of apolipoproteins that | | | | |
| | | | | | | | | | | | | | | are differentially | | | | |
| | | | | | | | | | | | | | | expressed. some | | | | |
| | | | | | | | | | | | | | | represent acute phase | | | | |
| | | | | | | | | | | | | | | proteins in the | | | | |
| | | | | | | | | | | | | | | response to | | | | |
| | | | | | | | | | | | | | | inflammatory stimuli. | | | | |
| | | | | | | | | | | | | | | one acts as a precursor | | | | |
| | | | | | | | | | | | | | | of the amyloid a | | | | |
| | | | | | | | | | | | | | | protein, a major | | | | |
| | | | | | | | | | | | | | | constituent of amyloid | | | | |
| | | | | | | | | | | | | | | fibrils for | | | | |
| X03479 | 1.00 | −1.29 | 13.98 | 9.47 | 18.92 | 17.89 | 104.27 | 16.64 | 92.13 | 20.20 | serum amyloid | X03479 | serum amyloid | serum amyloid a | 7 23.5 | Extracellular | | |
| | | | | | | | | | | | A3; Saa3 | | a-3 protein | protein (saa) | cM | Protein | | |
| | | | | | | | | | | | | | precursor | constitute a family of | | | | |
| | | | | | | | | | | | | | | apolipoproteins that | | | | |
| | | | | | | | | | | | | | | are differntially | | | | |
| | | | | | | | | | | | | | | expressed. some | | | | |
| | | | | | | | | | | | | | | family members | | | | |
| | | | | | | | | | | | | | | represent acute phase | | | | |
| | | | | | | | | | | | | | | proteins in thw | | | | |
| | | | | | | | | | | | | | | response to | | | | |
| | | | | | | | | | | | | | | inflammatory stimuli. | | | | |
| | | | | | | | | | | | | | | one of these acts as a | | | | |
| | | | | | | | | | | | | | | of the amide | | | | |
| U73004 | 1.00 | 4.48 | 15.01 | 7.85 | 9.35 | 4.84 | 56.63 | 17.17 | 86.24 | 20.61 | secretory | U73004 | antileuko- | acid-stable proteinase | | Proteolytic | | |
| | | | | | | | | | | | leukocyte | | proteinase | inhibitor with strong | | | | |
| | | | | | | | | | | | protease | | 1 precursor (alp) | affinities for typsin, | | | | |
| | | | | | | | | | | | inhibitor; Slpi | | (secretory | chymotrypsin, | | | | |
| | | | | | | | | | | | | | leukocyte | elastase, and cathepsin | | | | |
| | | | | | | | | | | | | | protease | g. may prevent | | | | |
| | | | | | | | | | | | | | inhibitor). | elastase-mediated | | | | |
| | | | | | | | | | | | | | | damage to oral and | | | | |
| | | | | | | | | | | | | | | possibly other mucosal | | | | |
| | | | | | | | | | | | | | | tissues. associated | | | | |
| | | | | | | | | | | | | | | with wound healing | | | | |
| | | | | | | | | | | | | | | due to its inhibition of | | | | |
| | | | | | | | | | | | | | | protease | | | | |
| M83219 | 1.00 | 8.84 | 25.35 | 7.26 | 17.08 | 10.37 | 45.22 | 2.88 | 62.94 | 10.24 | S100 calcium- | M83219 | calgranulin b | expressed by | 3 43.6 cM | Cytokine | | |
| | | | | | | | | | | | binding protein | | (migration | macrophages in | | | | |
| | | | | | | | | | | | A9 (calgranulin | | inhibitory factor- | acutely inflammated | | | | |
| | | | | | | | | | | | B); S100a9 | | related protein | tissues and in chronic | | | | |
| | | | | | | | | | | | | | 14) (mrp-14)(p14) | inflammations. seems | | | | |
| | | | | | | | | | | | | | (leukocyte 11 | to be an inhibitor of | | | | |
| | | | | | | | | | | | | | complex heavy | protein kinases. also | | | | |
| | | | | | | | | | | | | | chain) | expressed in epithelial | | | | |
| | | | | | | | | | | | | | | cells constititively or | | | | |
| | | | | | | | | | | | | | | induced during | | | | |
| | | | | | | | | | | | | | | dermatoses. may | | | | |
| | | | | | | | | | | | | | | interact with | | | | |
| | | | | | | | | | | | | | | components of | | | | |
| U27267 | 1.00 | 1.00 | 9.77 | 7.32 | 6.29 | 5.29 | 46.55 | 3.12 | 52.23 | 16.41 | small inducible | U27267 | small inducible | may participate in the | 5 53.0 cM | Cytokine | | |
| | | | | | | | | | | | cytokine B | | cytokine b5 | recruitment of | | | | |
| | | | | | | | | | | | subfamily, | | precursor | inflammatory cells by | | | | |
| | | | | | | | | | | | member 5; Scyb5 | | (cytokine lix). | injured or infected | | | | |
| | | | | | | | | | | | | | | tissue. involved in | | | | |
| | | | | | | | | | | | | | | neutrophil activation | | | | |
| Msa.2129.0 | 1.00 | 2.49 | 9.94 | 3.74 | 4.24 | 3.09 | 49.39 | 3.66 | 45.88 | 11.97 | lipocalin 2;Lcn2 | W13166 | neutrophil | acuto phase forms a | 2 27.0 cM | Extracellular | | |
| | | | | | | | | | | | | | gelatinase- | covalently linked, | | Protein | | |
| | | | | | | | | | | | | | associated | disulfide-bridged | | Protein | | |
| | | | | | | | | | | | | | lipocalin | heterodimer with the | | | | |
| | | | | | | | | | | | | | precursor (ngal) | 92 kd type v | | | | |
| | | | | | | | | | | | | | (p25) (sv-40 | collagenase (mmp-9). | | | | |
| | | | | | | | | | | | | | induced 24p3 | neutrophil gelatinase- | | | | |
| | | | | | | | | | | | | | protein). | associated lipocalin | | | | |
| | | | | | | | | | | | | | | 2,25kda,found at | | | | |
| | | | | | | | | | | | | | | moderate levels, only | | | | |
| | | | | | | | | | | | | | | in breast and lung, | | | | |
| | | | | | | | | | | | | | | component of human | | | | |
| | | | | | | | | | | | | | | tear(lipophilic ligand | | | | |
| | | | | | | | | | | | | | | carrier protein | | | | |
| | | | | | | | | | | | | | | superfamily, lipocalin | | | | |
| | | | | | | | | | | | | | | family, kernal group), | | | | |
| | | | | | | | | | | | | | | modulator of | | | | |
| | | | | | | | | | | | | | | inflammation, | | | | |
| | | | | | | | | | | | | | | involved in the | | | | |
| J04596 | 1.00 | 1.16 | 6.78 | 5.34 | 3.06 | 1.86 | 25.23 | 2.49 | 37.61 | 11.33 | GRO1 oncogene; | J04596 | growth regulated | has chemotactic | 5 51.0 cM | Extracellular | | |
| | | | | | | | | | | | Gro1 | | protein precursor | activity for | | Protein | | |
| | | | | | | | | | | | | | (platelet-derived | neutrophils. | | | | |
| | | | | | | | | | | | | | growth factor- | contributes to | | | | |
| | | | | | | | | | | | | | inducible protein | neutrophil activation | | | | |
| | | | | | | | | | | | | | kc) (secretory | during inflammation | | | | |
| | | | | | | | | | | | | | protein n51) | (by similarity) | | | | |
| Z27231 | 1.00 | 2.87 | 3.52 | 2.37 | 10.54 | 8.59 | 22.63 | 6.04 | 32.08 | 4.73 | matrix | Z27231 | 92 kda type iv | regulator in matrix | 2 96.0 cM | Metabolic | | |
| | | | | | | | | | | | metalloproteinase | | collagenase | remodeling, expressed | | | | |
| | | | | | | | | | | | 9; Mmp9 | precursor (ec | in alvcolar | | | | | |
| | | | | | | | | | | | | | 3.4.24.35) | macrophages and | | | | |
| | | | | | | | | | | | | | (92 kda | granulocytes, key | | | | |
| | | | | | | | | | | | | | gelatinase) | regulator of growth | | | | |
| | | | | | | | | | | | | | (matrix | plate angiogenesis and | | | | |
| | | | | | | | | | | | | | metallo- | apoptosis of | | | | |
| | | | | | | | | | | | | | proteinase- | hypertrophic | | | | |
| | | | | | | | | | | | | | 9) (mmp-9) | chonddrocytes in mice, | | | | |
| | | | | | | | | | | | | | (gelatinase b) | and involved in the | | | | |
| | | | | | | | | | | | | | (gelb). | migrator; process | | | | |
| M15131 | 1.00 | 1.55 | 5.35 | 3.53 | 5.53 | 4 53 | 28.75 | 3.16 | 31.62 | 8.61 | interleukin I beta; | M15131 | interleukin-1 beta | produced by | 2 73.0 cM | Cytokine | | |
| | | | | | | | | | | | II1b | | precursor | macrophages and | | | | |
| | | | | | | | | | | | | | (i1-1 beta). | monocytes, it is | | | | |
| | | | | | | | | | | | | | | required for the | | | | |
| | | | | | | | | | | | | | | interaction between | | | | |
| | | | | | | | | | | | | | | antigen-presenting | | | | |
| | | | | | | | | | | | | | | cells and lymphocytes | | | | |
| | | | | | | | | | | | | | | initiating an immune | | | | |
| | | | | | | | | | | | | | | response. it can also | | | | |
| | | | | | | | | | | | | | | be produced from a | | | | |
| | | | | | | | | | | | | | | number of other cells | | | | |
| | | | | | | | | | | | | | | and is involved in a | | | | |
| | | | | | | | | | | | | | | diversity of | | | | |
| X66402 | 1.00 | −1.04 | 3.76 | 3.44 | 6.56 | 7.79 | 26.89 | 1.34 | 30.00 | 4.34 | matrix | X66402 | stromelysin-1 | the stromelysins are | 9 1.0 cM | Proteolytic | | |
| | | | | | | | | | | | metalloproteinase | | precursor (ec | metalloproteinase | | | | |
| | | | | | | | | | | | 3; Mmp3 | | 3.4.24.17)(matrix | enzymes (ec | | | | |
| | | | | | | | | | | | | | metallo- | 3.4.24.17) involved in | | | | |
| | | | | | | | | | | | | | proteinase-3) | the metabolism of | | | | |
| | | | | | | | | | | | | | (mmp-3) (transin- | components of the | | | | |
| | | | | | | | | | | | | | 1)(s1-1). | extracellular matrix | | | | |
| AA638539 | 1.00 | 1.84 | 7.52 | 3.14 | 1.82 | 0.25 | 14.69 | 4.30 | 26.78 | 8.36 | DNA segment, | AA638539 | | | 5 54.0 cM | EST; | | |
| | | | | | | | | | | | Chr 5, | | | | | Unknown | | |
| | | | | | | | | | | | Wayne State | | | | | | | |
| | | | | | | | | | | | University 111, | | | | | | | |
| | | | | | | | | | | | expressed; | | | | | | | |
| | | | | | | | | | | | D5Wsu111e | | | | | | | |
| Msa.27241.0 | 1.00 | 1.00 | 3.58 | 2.53 | 4.68 | 0.44 | 14.73 | 7.02 | 23.04 | 4.95 | insulin-like | AA066368 | insulin-like | igf-binding proteins | | | | |
| | | | | | | | | | | | growth factor | | growth factor | prolong the half-life of | | | | |
| | | | | | | | | | | | binding protein | | binding protein | the igfs and have been | | | | |
| | | | | | | | | | | | 4; Igfbp4 | | 4 precursor | shown to either inhibitor | | | | |
| | | | | | | | | | | | | | (igfbp-4) (ibp-4) | stimulate the | | | | |
| | | | | | | | | | | | | | (igf binding | growth promoting | | | | |
| | | | | | | | | | | | | | protein 4). | effects of the igfs on | | | | |
| | | | | | | | | | | | | | | cell culture. they after | | | | |
| | | | | | | | | | | | | | | the interaction of gifs | | | | |
| | | | | | | | | | | | | | | with their cell surface | | | | |
| | | | | | | | | | | | | | | receptors | | | | |
| M58288 | 1.00 | 1.00 | 4.46 | 2.99 | 2.92 | 1.92 | 23.86 | 0.91 | 21.79 | 6.27 | colony | M58288 | granulocyte | receptor for | 4 57.5 cM | Receptor | | |
| | | | | | | | | | | | stimulating factor | | colony | granulocyte colony- | | | | |
| | | | | | | | | | | | 3 receptor | | stimulating factor | stimulating factor (g- | | | | |
| | | | | | | | | | | | (granulocyte); | | receptor precursor | csf). in addition it may | | | | |
| | | | | | | | | | | | Csf3r | | (g-csf-r). | function in some | | | | |
| | | | | | | | | | | | | | | adhesion or | fi | | | |
| | | | | | | | | | | | | | | recognition events at | | | | |
| | | | | | | | | | | | | | | the cell surface | a | | | |
| M99054 | 1 00 | 1.38 | 2.39 | 1.33 | 14.56 | 12.33 | 20.35 | 11.20 | 21.54 | 4.48 | acid phosphate 5, | M99054 | tartrate-resistant | aka trap (in mouse, not | 9 6.0 cM | Intracellular | | |
| | | | | | | | | | | | tartrate resistant | | acid phosphatase | human) -- a lysosomal | | | | |
| | | | | | | | | | | | (TRAP): Acp5 | | type 5 precursor | enzyme and marker of | | | | |
| | | | | | | | | | | | | | (cc 3.1.3.2)(tr-ap) | osteoclasts. | | | | |
| | | | | | | | | | | | | | (tartrate-resistant | | | | | |
| | | | | | | | | | | | | | acid atpase) | | | | | |
| | | | | | | | | | | | | | (tratnase) | | | | | |
| M83218 | 1.00 | 9.16 | 8.77 | 1.81 | 9.30 | 6.50 | 15.39 | 2.10 | 20.18 | 0.81 | S100 calcium | M83218 | calgranulin a | expressed by | 3 43.6 cM | Cytokine | | |
| | | | | | | | | | | | binding protein | | (migration | macrophages in | | | | |
| | | | | | | | | | | | A8 (calgranulin | | inhibitory factor- | chronic | | | | |
| | | | | | | | | | | | A); S100a8 | | related protein 8) | inflammations. also | | | | |
| | | | | | | | | | | | | | (mrp-8) (p8) | expressed in epithelial | | | | |
| | | | | | | | | | | | | | (leukocyte 11 | cells constitutively or | | | | |
| | | | | | | | | | | | | | complex light | induced during | | | | |
| | | | | | | | | | | | | | chain) | dermatoses. may | | | | |
| | | | | | | | | | | | | (chemotactic | interact with | | | | | |
| | | | | | | | | | | | | | cytokine cp-10) | components of the | | | | |
| | | | | | | | | | | | | | (pro-inflamma- | intermediate filaments | | | | |
| | | | | | | | | | | | | | tory s100 | in monocytes and | | | | |
| | | | | | | | | | | | | | cytokine). | | | | | |
| V00755 | 1.00 | −1.98 | 2.82 | 2.28 | 5.88 | 4.16 | 18.69 | 2.71 | 19.18 | 3.63 | tissue inhibitor of | V00755 | metalloproteinase | complexes with | X 6.2 cM | Proteolytic | | |
| | | | | | | | | | | | metalloproteinase; | | inhibitor 1 | metalloproteinases | | | | |
| | | | | | | | | | | | Timp | | precursor (timp-1) | (such as collagenases) | | | | |
| | | | | | | | | | | | | | (erythroid | and irreversibly | | | | |
| | | | | | | | | | | | | | potentiating | inactivates them. | | | | |
| | | | | | | | | | | | | | activity) (epa) | mediates | | | | |
| | | | | | | | | | | | | | (tissue inhibitor | erythropoiesis in vitro; | | | | |
| | | | | | | | | | | | | | of metallopro- | but, unlike i1-3, it is | | | | |
| | | | | | | | | | | | | | teinases) | species-specific, | | | | |
| | | | | | | | | | | | | | (collagenase | stimulating growth | | | | |
| | | | | | | | | | | | | | inhibitor 16c8 | and differentiation of | | | | |
| | | | | | | | | | | | | | fibroblast) (tpa- | only human and | | | | |
| | | | | | | | | | | | | | induced protein) | | | | | |
| | | | | | | | | | | | | | (tpa-s1). | | | | | |
| M19681 | 1.00 | −1.30 | 6.02 | 2.91 | 3.70 | 2.69 | 36.38 | 2.49 | 18.47 | 6.24 | small inducible | M19681 | small inducible | scya2 is expressed in | 11 46.5 | Cytokine | | |
| | | | | | | | | | | | cytokine A2; | | cytokine a2 | activated mast cells, | cM | | | |
| | | | | | | | | | | | Scya2 | | precursor | macrophages, and | | | | |
| | | | | | | | | | | | | | (monocyte | nerve cells . . . a b | | | | |
| | | | | | | | | | | | | | chemotactic pro- | chemokine | | | | |
| | | | | | | | | | | | | | tein 1) (mcp-1) | corresponding to the | | | | |
| | | | | | | | | | | | | | (monocyte | monocyte chemotactic | | | | |
| | | | | | | | | | | | | | chemoattractant | protein, induced by | | | | |
| | | | | | | | | | | | | | protein-1) | pdgf. chemotactic | | | | |
| | | | | | | | | | | | | | (platelet- | factor that attracts | | | | |
| | | | | | | | | | | | | | derived growth | monocytes and | | | | |
| | | | | | | | | | | | | | factor-inducible | basophils, but not | | | | |
| | | | | | | | | | | | | | protein je). | neutrolphils or | | | | |
| | | | | | | | | | | | | | | ensinophils, augments | | | | |
| | | | | | | | | | | | | | | monocyte infiltrates, | | | | |
| | | | | | | | | | | | | | | like psoriasis | | | | |
| | | | | | | | | | | | | | | rheumatoid arthritis, | | | | |
| | | | | | | | | | | | | | | and atherosclerosis. | | | | |
| | | | | | | | | | | | | | | may be involved in the | | | | |
| | | | | | | | | | | | | | | recruitment of | | | | |
| | | | | | | | | | | | | | | monocytes into the | | | | |
| | | | | | | | | | | | | | | arterial wall during the | | | | |
| | | | | | | | | | | | | | | disease process of | | | | |
| | | | | | | | | | | | | | | atherosclerosis, binds | | | | |
| M60429 | 1.00 | 1.00 | 5.74 | 2.53 | 3.84 | 0.70 | 3.70 | 1.99 | 16.94 | 1.62 | immunoglobulin | M60429 | | | 12 58.0 | | | |
| | | | | | | | | | | | heavy chain 4 | | | | cM | | | |
| | | | | | | | | | | | (serum | | | | | | | |
| | | | | | | | | | | | IgG1); Igh-4 | | | | | | | |
| Msa.34452.0 | 1.00 | 1.00 | 1.19 | 0.94 | 8.43 | 9.68 | 7.55 | 1.29 | 15.96 | 6.85 | insulin-like | AA117813 | insulin-like | igf-binding proteins | | Extracellular | | |
| | | | | | | | | | | | growth factor | | growth factor | prolong the half-life of | | Protein | | |
| | | | | | | | | | | | binding protein | | binding protein 4 | the igfs and have been | | | | |
| | | | | | | | | | | | 4; Igfbp4 | | precursor (igfbp- | shown to either inhibitor | | | | |
| | | | | | | | | | | | | | 4) (ibp-4) (igf | or stimulate the | | | | |
| | | | | | | | | | | | | | binding | growth promoting | | | | |
| | | | | | | | | | | | | | protein 4). | effects of the igfs on | | | | |
| | | | | | | | | | | | | | | cell culture. they alter | | | | |
| | | | | | | | | | | | | | | the interaction of igfs | | | | |
| | | | | | | | | | | | | | | with their cell surface | | | | |
| | | | | | | | | | | | | | | receptors | | | | |
| X94353 | 1.00 | 4.36 | 6.28 | 1.96 | 3.34 | 0.80 | 3.60 | 1.19 | 14.16 | 0.37 | cathelin-like | X94353 | cathelin-related | acts as a potent | 9 61.0 cM | Metabolic | | |
| | | | | | | | | | | | protein; Cnlp | | antimicrobial | antimicrobial peptide. | | | | |
| | | | | | | | | | | | | | peptide precursor | | | | | |
| | | | | | | | | | | | | | (cramp) (cathelin- | | | | | |
| | | | | | | | | | | | | | like protein) (clp) | | | | | |
| Msa.38664.0 | 1.00 | 1 00 | 3.40 | 0.91 | 2.76 | 1.76 | 5.71 | 1.59 | 13.90 | 3.18 | | AA144469 | | | | EST; | | |
| | | | | | | | | | | | | | | | | Unknown | | |
| W49204 | 1.00 | 1.24 | 7.53 | 2.19 | 6.24 | 4.95 | 7.85 | 4.84 | 13.67 | 4.24 | glypican 1; Gpc1 | W49204 | | | | | | |
| X66473 | 1.00 | 1.79 | 0.70 | 1.41 | 2.92 | 4.00 | 10.73 | 1.24 | 13.46 | 1.70 | matrix | X66473 | collagenase 3 | degrades collagen type | 9 | Proteolytic | | |
| | | | | | | | | | | | metalloproteinase | | precursor (ec | i. does not act on | | | | |
| | | | | | | | | | | | 13; Mmp13 | | 3.4.24.) (matrix | gelatin or casein. | | | | |
| | | | | | | | | | | | | | metallopro- | could have a role in | | | | |
| | | | | | | | | | | | | | teinase-13) | tumoral process. | | | | |
| | | | | | | | | | | | | | (mmp-13). | regulator of matrix | | | | |
| | | | | | | | | | | | | | | remodeling | | | | |
| | | | | | | | | | | | | | | component of the | | | | |
| | | | | | | | | | | | | | | mmp cluster, | | | | |
| | | | | | | | | | | | | | expressed in the | | | | | |
| | | | | | | | | | | | | | | synovial membrane | | | | |
| | | | | | | | | | | | | | | and synovial | | | | |
| L37297 | 1.00 | 2.53 | 6.23 | 1.63 | 2.38 | 0.66 | 3.08 | 1.11 | 12.74 | 1.56 | neurrophilic | L37297 | | a novel myeloid- | | Intracellular | | |
| | | | | | | | | | | | granule | | | specific granule | | Protein | | |
| | | | | | | | | | | | protein; Ngp | | | protein related to | | | | |
| | | | | | | | | | | | | | | porcine cathelin, but | | | | |
| | | | | | | | | | | | | | | showing important | | | | |
| | | | | | | | | | | | | | | structural differences. | | | | |
| | | | | | | | | | | | | | | this may represent the | | | | |
| | | | | | | | | | | | | | | first isolated member | | | | |
| | | | | | | | | | | | | | | of a new cystatin | | | | |
| | | | | | | | | | | | | | | family, more | | | | |
| | | | | | | | | | | | | | | importantly, the small | | | | |
| | | | | | | | | | | | | | | size of the | | | | |
| X91144 | 1.00 | 2.10 | 3.53 | 1.24 | 4.04 | 2.92 | 10.33 | 0.54 | 12.68 | 2.95 | selectin, platelet | X91144 | p-scleetin | binds to p-, e- and 1- | 5 64.0 cM | Extracellular | | |
| | | | | | | | | | | | (p-selectin) | | glycoprotein | selectins. the calcium- | | Protein | | |
| | | | | | | | | | | | ligand; Selp1 | | ligand 1 precursor | dependent high | | | | |
| | | | | | | | | | | | | | (psg1-1) (selectin | affinity interaction | | | | |
| | | | | | | | | | | | | | p ligand) | with p-selectin | | | | |
| | | | | | | | | | | | | | | mediates the tethering | | | | |
| | | | | | | | | | | | | | | and rolling of | | | | |
| | | | | | | | | | | | | | | neutrolphils and t- | | | | |
| | | | | | | | | | | | | | | lymphocytes on | | | | |
| X54542 | 1.00 | 1.37 | 2.99 | 1.71 | 1.36 | 0.24 | 8.15 | 2.35 | 12.39 | 3.14 | interleukin 6; I16 | X54542 | interleukin-6 | i16 may be the most | 5 17.0 cM | Cytokine | | |
| | | | | | | | | | | | | | precursor (i1-6) | extremely pleiotropic | | | | |
| | | | | | | | | | | | | | (interleukin | of cytokines, with a | | | | |
| | | | | | | | | | | | | | hp-1)(b- | broad range of | | | | |
| | | | | | | | | | | | | | cell hybridoma | activities on different | | | | |
| | | | | | | | | | | | | | growth factor). | cell types. | | | | |
| X81627 | 1.00 | 1.92 | 3.27 | 1.14 | 2.57 | 0.96 | 12.19 | 0.13 | 11.79 | 2.91 | lipocalin 2; Lcn2 | X81627 | neutrophil | capable of carrying | 2 27.0 cM | Extracellular | | |
| | | | | | | | | | | | | | gelatinase- | small lipopphilic | | Protein | | |
| | | | | | | | | | | | | | associated | molecules like retinol, | | | | |
| | | | | | | | | | | | | | lipocalin | steroids, and odorants. | | | | |
| | | | | | | | | | | | | | precursor (ngal) | | | | | |
| | | | | | | | | | | | | | (p25) (sv-40 | | | | | |
| | | | | | | | | | | | | | induced 24p3 | | | | | |
| ET62052 | 1.00 | 1.06 | 5.14 | 2.14 | 3.73 | 0.87 | 3.52 | 1.29 | 11.69 | 0.43 | immunoglobulin | ET62052 | | | 12 58.0 | | | |
| | | | | | | | | | | | heavy chain 4 | | | | cM | | | |
| | | | | | | | | | | | (serum | | | | | | | |
| | | | | | | | | | | | IgG1 Igh-4 | | | | | | | |
| W44075 | 1.00 | 1.61 | 5.43 | 2.30 | 3.25 | 1.18 | 3.35 | 1.14 | 11.54 | 2.21 | myeloperoxidase; | W44075 | myeloperoxidase | this enzyme is present | 11 49.0 | Intracellular | | |
| | | | | | | | | | | | Mpo | | precursor (ec | in primary granules of | cM | Protein | | |
| | | | | | | | | | | | | | 1.11.1.7)(mpo). | neutrophilic | | | | |
| | | | | | | | | | | | | | | granulocytes and plays | | | | |
| | | | | | | | | | | | | | | a major role in the | | | | |
| | | | | | | | | | | | | | | oxygen-dependent | | | | |
| | | | | | | | | | | | | | | microbicidal system of | | | | |
| | | | | | | | | | | | | | | granulocytes | | | | |
| Msa.6242.0 | 1.00 | −1.69 | −1.10 | 2.37 | 4.33 | 3.18 | 8.04 | 2.31 | 10.59 | 1.01 | cathepsin K; Ctsk | W13263 | cathepsin k | closely involved in | 3 47.9 cM | Proteolytic | | |
| | | | | | | | | | | | | | precursor (ec | osteoclastic bone | | | | |
| | | | | | | | | | | | | | 3.4.22.38). | resorption and may | | | | |
| | | | | | | | | | | | | | | participate partially in | | | | |
| | | | | | | | | | | | | | | the disorder of bone | | | | |
| | | | | | | | | | | | | | | remodeling. displays | | | | |
| | | | | | | | | | | | | | | potent endoprotease | | | | |
| | | | | | | | | | | | | | | activity against | | | | |
| | | | | | | | | | | | | | | fibrinogen at acid ph. | | | | |
| | | | | | | | | | | | | | | may play an important | | | | |
| | | | | | | | | | | | | | | role in extracellular | | | | |
| | | | | | | | | | | | | | | matrix degrade | | | | |
| U59488 | 1.00 | 1.09 | 2.63 | 0.99 | 2.49 | 0.01 | 12.29 | 1.48 | 9.98 | 2.50 | neutrophil | U59488 | neutrophil cytosol | nadph oxidase consists | 15 47.2 | Inreacellular | | |
| | | | | | | | | | | | cytosolic | | factor 4 (ncf-4) | of proteins p47-phox, | cM | Protein | | |
| | | | | | | | | | | | factor 4; Ncf4 | | (neutrophil nadph | p67-phox, p4-phox, | | | | |
| | | | | | | | | | | | | | oxidase factor 4) | and a small regulatory | | | | |
| | | | | | | | | | | | | | (p40-phox) | g protein. p4-phox is | | | | |
| | | | | | | | | | | | | | (p40phox). | not required for | | | | |
| | | | | | | | | | | | | | | oxidase activity and | | | | |
| | | | | | | | | | | | | | | has been proposed to | | | | |
| | | | | | | | | | | | | | | have a regulatory | | | | |
| | | | | | | | | | | | | | | function | | | | |
| Z12297 | 1.00 | −1.13 | 2.36 | 1.52 | 2.47 | 4.30 | 14.05 | 0.57 | 9.42 | 1.82 | small inducible | Z12297 | small inducible | chemotactic factor | 11 46.5 | Cytokine | | |
| | | | | | | | | | | | cytokine A7; | | cytokine a7 | that attracts | cM | | | |
| | | | | | | | | | | | Scya7 | | precursor | monocytes and | | | | |
| | | | | | | | | | | | | | (monocyte | eosinophils, but not | | | | |
| | | | | | | | | | | | | | chemotactic | neutrophils, augments | | | | |
| | | | | | | | | | | | | | protein 3) | monocyte anti-tumor | | | | |
| | | | | | | | | | | | | | (mcp-3) | activity (by similarity). | | | | |
| | | | | | | | | | | | | | (monocyte | also induces release of | | | | |
| | | | | | | | | | | | | | chemoattrac- | gelatinase b. binds to | | | | |
| | | | | | | | | | | | | | tant protein 3) | ccr1, ccr2, ccr3. | | | | |
| | | | | | | | | | | | | | (intererine/ | | | | | |
| | | | | | | | | | | | | | chemokina | | | | | |
| | | | | | | | | | | | | | more) (fic | | | | | |
| C76739 | 1.00 | 1.00 | 1.85 | 1.03 | 2.46 | 3.48 | 12.67 | 2.14 | 9.30 | 2.34 | macrophage | C76739 | | a type ii | 6 56.5 cM | Cell Surface | | |
| | | | | | | | | | | | C-type | | | transmembrane | | Protein | | |
| | | | | | | | | | | | lectin; Mpc1 | | | protein with a single | | | | |
| | | | | | | | | | | | | | | extracellular c-type | | | | |
| | | | | | | | | | | | | | | lectin domain, | | | | |
| | | | | | | | | | | | | | | expressed in cell lines | | | | |
| | | | | | | | | | | | | | | and normal mouse | | | | |
| | | | | | | | | | | | | | | tissues in a | | | | |
| | | | | | | | | | | | | | | macrophage-restricted | | | | |
| | | | | | | | | | | | | | | manner | | | | |
| U60438 | 1.00 | 1.09 | 2.36 | 0.99 | 2.45 | 1.31 | 17.95 | 6.58 | 8.54 | 2.10 | serum amyloid A | U60438 | serum amyloid | saa1, saa2, and saa3 | 7 23.5 cM | Other | | |
| | | | | | | | | | | | 2; Saa2 | | a-2 protein | encode acute phase | | | | |
| | | | | | | | | | | | | | precursor | response proteins in | | | | |
| | | | | | | | | | | | | | [contains: | response to | | | | |
| | | | | | | | | | | | | | amyloid protein | inflammatory stimuli. | | | | |
| | | | | | | | | | | | | | a (amyloid | acts as a precursor of | | | | |
| | | | | | | | | | | | | | fibril protein aa)]. | the amyloid a protein | | | | |
| | | | | | | | | | | | | | | that is a major | | | | |
| | | | | | | | | | | | | | | constituent of amyloid | | | | |
| | | | | | | | | | | | | | | fibrils formed in | | | | |
| | | | | | | | | | | | | | | secondary | | | | |
| X94444 | 1.00 | −1.35 | −0.12 | 1.31 | 3.13 | 2.06 | 5.71 | 1.36 | 8.43 | 0.90 | cathepsin K; Ctsk | X94444 | cathepsin k | cathepsin k (ec | 3 47.9 cM | Proteolytic | | |
| | | | | | | | | | | | | | precursor (ec | 3.4.22.38), encoded in | | | | |
| | | | | | | | | | | | | | 3.4.22.38). | the mouse by ctsk, is | | | | |
| | | | | | | | | | | | | | | implicated in bone | | | | |
| | | | | | | | | | | | | | | resorption. expression | | | | |
| | | | | | | | | | | | | | | is predominantly in | | | | |
| | | | | | | | | | | | | | | osteoclasts; embryonic | | | | |
| | | | | | | | | | | | | | | expression also takes | | | | |
| | | | | | | | | | | | | | | place in some | | | | |
| | | | | | | | | | | | | | | hypertrophic | | | | |
| | | | | | | | | | | | | | | chondrocytes of | | | | |
| | | | | | | | | | | | | | | growth cartilages | | | | |
| M73748 | 1.00 | −1.54 | 1.94 | 0.96 | 2.06 | 0.54 | 9.36 | 1.95 | 8.21 | 1.69 | glycoprotein 38; | M73748 | glycoprotein 38 | | | Cell Surface | | |
| | | | | | | | | | | | Gp38 | | precursor (gp38) | | | Protein | | |
| | | | | | | | | | | | | | (ots-8). | | | | | |
| X83601 | 1.00 | 1.04 | 0.95 | 1.04 | 1.89 | 3.72 | 14.68 | 1.06 | 8.18 | 2.71 | pentaxin related | X83601 | pentaxin-related | a tnf stimulated gene. | 3 33.8 cM | Extracellular | | |
| | | | | | | | | | | | gene; Ptx3 | | protein ptx3 | produced outside of | | Protein | | |
| | | | | | | | | | | | | | precursor (tumor | the liver, increased | | | | |
| | | | | | | | | | | | | | necrosis factor- | levels of expression | | | | |
| | | | | | | | | | | | | | inducible protein | are induced by 1ps. | | | | |
| | | | | | | | | | | | | | tsg 14) | | | | | |
| X96639 | 1.00 | −1.20 | 1.41 | 0.88 | 1.95 | 0.27 | 4.72 | 1.16 | 8.11 | 2.12 | exostoses | X96639 | exostosin-1 | appears to be a tumor | 15 26.55 | Other | | |
| | | | | | | | | | | | (multiple) | | (putative tumor | suppressor. | cM | | | |
| | | | | | | | | | | | 1; Ext 1 | | | suppressor protein | | | | |
| | | | | | | | | | | | | | | ext1) (multiple | | | | |
| | | | | | | | | | | | | | | exostoses protein 1). | | | | |
| CR0103 | 1.00 | −1.06 | 1.60 | 0.88 | 3 45 | 2.25 | 8.46 | 0.69 | 7.56 | 1.13 | plastin 2, L; Pls2 | CR0103 | 1-plastin | actin-bundling protein. | | Intracellular | | |
| | | | | | | | | | | | | | | (lymphocyte | | | Protein | |
| | | | | | | | | | | | | | | cytosolic protein 1) | | | | |
| | | | | | | | | | | | | | | (1cp-1) ((65 kda | | | | |
| | | | | | | | | | | | | | | macrophage | | | | |
| | | | | | | | | | | | | | | protein)(np65) | | | | |
| M25324 | 1.00 | 1.00 | 1.80 | 0.68 | 1.26 | 0.26 | 6.55 | 1.35 | 7.55 | 2.74 | selectin, | M25324 | 1-selectin | cell surface adhesion | 1 86.6 cM | Cell Surface | | |
| | | | | | | | | | | | lymphocyte; | | precursor | protein, mediate the | | Protein | | |
| | | | | | | | | | | | Sell | | (lymph node | | | | | |
| | | | | | | | | | | | | | homing receptor) | adherence of | | | | |
| | | | | | | | | | | | | | (leukocyte | | | | | |
| | | | | | | | | | | | | | adhesion | lymphocytes to | | | | |
| | | | | | | | | | | | | | molecule-1)(1am- | endothelial cells of | | | | |
| | | | | | | | | | | | | | 1) (ly-22) | | | | | |
| | | | | | | | | | | | | | (lymphocyte | high endothelial | | | | |
| | | | | | | | | | | | | | surface mel-14 | venules in peripheral | | | | |
| | | | | | | | | | | | | | antigen) | | | | | |
| | | | | | | | | | | | | | (leukocyte- | lymph nodes. | | | | |
| | | | | | | | | | | | | | endothel cell | | | | | |
| | | | | | | | | | | | | | adhesion | | | | | |
| | | | | | | | | | | | | | molecule | | | | | |
| | | | | | | | | | | | | | 1)(lacam1) | | | | | |
| | | | | | | | | | | | | | (cd621) | | | | | |
| C80638 | 1.00 | −1.07 | 0.91 | 0.65 | 1.97 | 0.69 | 7.94 | 3.14 | 7.51 | 1.81 | EST, Unknown | C80638 | | | | EST; | | |
| | | | | | | | | | | | | | | | | | Unknown | |
| Msa.24575.0 | 1.00 | 1.03 | 0.77 | 0.72 | 2.95 | 0.34 | 6.02 | 0.16 | 7.13 | 1.48 | EST, | WR2261 | | | | EST; | | |
| | | | | | | | | | | | | | | | | | Unknown | |
| Msa.1271.0 | 1.00 | 1.60 | 2.92 | 0.86 | 1.26 | 0.15 | 1.35 | 0.13 | 6.94 | 0.84 | lactotransfernin; | 303298 | lactotransferrin | transferrins are iron | 9 61.0 cM | Intracellular | | |
| | | | | | | | | | | | Ltf | | precursor | binding transport | | Protein | | |
| | | | | | | | | | | | | | (lactoferrin). | proteins which can | | | | |
| | | | | | | | | | | | | | | bind two atoms of | | | | |
| | | | | | | | | | | | | | | ferric iron in | | | | |
| | | | | | | | | | | | | | | association with the | | | | |
| | | | | | | | | | | | | | | binding of an anion, | | | | |
| | | | | | | | | | | | | | | usually bicarbonate | | | | |
| AA200615 | 1.00 | −1.21 | 0.51 | 1.06 | 2.69 | 0.23 | 4.76 | 1.05 | 6.94 | 0.92 | homologue of | AA200615 | | patent held by quark | | Other | | |
| | | | | | | | | | | | GENESEQN:Z- | | | | | | | |
| | | | | | | | | | | | 36322 | | | biotech, inc. - | | | | |
| | | | | | | | | | | | Mechanical stress | | | identification of stress | | | | |
| | | | | | | | | | | | induced cDNA | | | induced genes for | | | | |
| | | | | | | | | | | | encoding | | | | | | | |
| | | | | | | | | | | | protein 608 | | | determining risk and | | | | |
| | | | | | | | | | | | | | | preventing, treating or | | | | |
| | | | | | | | | | | | | | | controlling | | | | |
| | | | | | | | | | | | | | | osteoporosis | | | | |
| Msa.136.0 | 1.00 | −1.02 | 1.05 | 0.73 | 2.34 | 1.16 | 4.34 | 0.20 | 6.84 | 1.15 | interleukin 2 | U21795 | cytokine receptor | common subunit for | X 38.0 cM | | | |
| | | | | | | | | | | | receptor, gamma | | common gamma | the receptors for a | | | | |
| | | | | | | | | | | | chain; I12rg | | chain precursor | variety of interleukins. | | | | |
| | | | | | | | | | | | | | | | (gamma-c) | | | |
| | | | | | | | | | | | | | | (interleukin-2 | | | | |
| | | | | | | | | | | | | | | receptor gamma | | | | |
| | | | | | | | | | | | | | | chain) (i1-2r gamma | | | | |
| | | | | | | | | | | | | | | chain)(n64) | | | | |
| Msa.739.0 | 1.00 | −1.82 | −1.01 | 2.43 | 0.89 | 2.70 | 6.58 | 0.93 | 6.75 | 0.72 | haptoglobin; Hp | M96827 | | haptoglobin combines | 8 55.0 cM | Extracellular | | |
| | | | | | | | | | | | | | | with free plasma | | Protein | | |
| | | | | | | | | | | | | | | hemoglobin, | | | | |
| | | | | | | | | | | | | | | preventing loss of iron | | | | |
| | | | | | | | | | | | | | | through the kidneys | | | | |
| | | | | | | | | | | | | | | and protecting the | | | | |
| | | | | | | | | | | | | | | kidneys from damage | | | | |
| | | | | | | | | | | | | | | by hemoglobin, while | | | | |
| | | | | | | | | | | | | | | making the | | | | |
| | | | | | | | | | | | | | | hemoglobin, accessible | | | | |
| | | | | | | | | | | | | | | to | | | | |
| D37837 | 1.00 | 1.26 | 1.48 | 0.88 | 2.18 | 0.80 | 5.85 | 1.31 | 6.34 | 1.47 | plastin 2, L; Pls2 | D37837 | 1-plastin | actin-bundling protein. | | Structural | | |
| | | | | | | | | | | | | | | (lymphocyte | | | Protein | |
| | | | | | | | | | | | | | | cytosolic protein 1) | | | | |
| | | | | | | | | | | | | | | (lcp-1) (65 kda | | | | |
| | | | | | | | | | | | | | | macrophage | | | | |
| | | | | | | | | | | | | | | protein)(nn65) | | | | |
| U17961 | 1.00 | 1.15 | 4.42 | 0.90 | 3.16 | 1.65 | 7.12 | 1.45 | 6.33 | 1.79 | scr associated in | U17961 | | | | | | |
| | | | | | | | | | | | mitosis, 68 kDa; | | | | | | | |
| | | | | | | | | | | | Sam68 | | | | | | | |
| U83903 | 1.00 | −1.40 | 0.77 | 1.17 | 1.33 | 3.57 | 8.40 | 3.12 | 6.29 | 0.55 | tumor necrosis | U83903 | | induced in vitro in | 2 | Regulatory | | |
| | | | | | | | | | | | factor induced | | | several cell types by | | | | |
| | | | | | | | | | | | protein 6; | | | proinflammatory | | | | |
| | | | | | | | | | | | Tnfip6 | | | cytokines, and in vivo | | | | |
| | | | | | | | | | | | | | | in pathological | | | | |
| | | | | | | | | | | | | | | conditions such as | | | | |
| | | | | | | | | | | | | | | rheumatoid arthritis. | | | | |
| | | | | | | | | | | | | | | interacts with link | | | | |
| | | | | | | | | | | | | | | protein and aggrecan. | | | | |
| | | | | | | | | | | | | | | involved in matrix | | | | |
| | | | | | | | | | | | | | | dissociation and | | | | |
| | | | | | | | | | | | | | | regulated by ph | | | | |
| M59378 | 1.00 | 1.72 | 2.24 | 0.60 | 2.28 | 1.03 | 7.57 | 0.87 | 6.26 | 1.20 | tumor necrosis | M59378 | tumor necrosis | encodes the larger of | 4 75.5 cM | Receptor | | |
| | | | | | | | | | | | factor receptor | | factor receptor 2 | twp receptors for the | | | | |
| | | | | | | | | | | | superfamily, | | precursor (tnf-r2) | tumor necrosis factor. | | | | |
| | | | | | | | | | | | member 1b; | | (p75) | its expression is | | | | |
| | | | | | | | | | | | Tnfrsf1b | | | regulated by external | | | | |
| | | | | | | | | | | | | | | factors. a tnfrsf1b | | | | |
| | | | | | | | | | | | | | | targeted null mutation | | | | |
| | | | | | | | | | | | | | | shows normal t-cell | | | | |
| | | | | | | | | | | | | | | development and | | | | |
| | | | | | | | | | | | | | | activity, but is | | | | |
| | | | | | | | | | | | | | | resistant to inf- | | | | |
| | | | | | | | | | | | | | | induced cell death | | | | |
| U16985 | 1.00 | 1.02 | 1.86 | 0.93 | 1.83 | 0.19 | 5.30 | 0.24 | 6.23 | 1.04 | lymphotoxin | U16985 | lymphotoxin-beta | member of the tnf | 17 19.06 | Cytokine | | |
| | | | | | | | | | | | B; Ltb | | (1t beta)(tumor | ligand family isolated | cM | | | |
| | | | | | | | | | | | | | necrosis factor c). | from human t cells. | | | | |
| | | | | | | | | | | | | | | only found on the cell | | | | |
| | | | | | | | | | | | | | | surface, where it | | | | |
| | | | | | | | | | | | | | | forms a 2:1 trimeric | | | | |
| | | | | | | | | | | | | | | complex with | | | | |
| | | | | | | | | | | | | | | lymphotoxin a, | | | | |
| | | | | | | | | | | | | | | constitutively | | | | |
| | | | | | | | | | | | | | | expressed in lymphoid | | | | |
| | | | | | | | | | | | | | | and hematopoietic | | | | |
| | | | | | | | | | | | | | | tissues, maximal in | | | | |
| | | | | | | | | | | | | | | thymic medulla and | | | | |
| | | | | | | | | | | | | | | splenic white pulp | | | | |
| | | | | | | | | | | | | | | may play a specific | | | | |
| | | | | | | | | | | | | | | role in immune | | | | |
| | | | | | | | | | | | | | | response regulation. | | | | |
| | | | | | | | | | | | | | | provides the | | | | |
| | | | | | | | | | | | | | | membrane anchor for | | | | |
| | | | | | | | | | | | | | | the attachment of the | | | | |
| Msa.1700.0 | 1.00 | 2.40 | −0.32 | 0.82 | 2.12 | 3.77 | 5.59 | 2.04 | 5.93 | 0.88 | phospholipase A2 | U34277 | platelet-activating | modulates the action | | Cytokine | | |
| | | | | | | | | | | | group VII | | factor | of platelet-activating | | | | |
| | | | | | | | | | | | (platelet- | | acetylhydrolase | factor (paf) by | | | | |
| | | | | | | | | | | | activating factor | | precursor (ec) | hydrolyzing the sn-2 | | | | |
| | | | | | | | | | | | acetylhydrolase, | | 3.1.1.47) (paf | ester bond to yield the | | | | |
| | | | | | | | | | | | plasma); Pla2g7 | | acetylhydrolase) | biologically inactive | | | | |
| | | | | | | | | | | | | | (paf2- | lyso-paf. has a | | | | |
| | | | | | | | | | | | | | acylhydrolase) | specificity for | | | | |
| | | | | | | | | | | | | | (1d1-associated | substrates with a short | | | | |
| | | | | | | | | | | | | | phospholipase a2) | residue at the the sn-2 | | | | |
| | | | | | | | | | | | | | (1d1-pla(2)) | position. it is inactive | | | | |
| | | | | | | | | | | | | | (2-acetyl-1- | against lo | | | | |
| | | | | | | | | | | | | | alkylglycero- | | | | | |
| | | | | | | | | | | | | | phosphocholinc | | | | | |
| | | | | | | | | | | | | | csterasc) | | | | | |
| | | | | | | | | | | | | | (1-alkyl-2- | | | | | |
| | | | | | | | | | | | | | acetylglycero- | | | | | |
| | | | | | | | | | | | | | phosph | | | | | |
| AA059883 | 1.00 | −1.03 | 0.28 | 1.16 | 3.05 | 4.37 | 8.59 | 2.30 | 5.91 | 0.99 | angiopoietin | AA059883 | | the region of this | | Hemostasis | | |
| | | | | | | | | | | | related | | | genomic sequence that | | | | |
| | | | | | | | | | | | | | | contains this sequence | | | | |
| | | | | | | | | | | | | | | is listed as | | | | |
| | | | | | | | | | | | | | | angiopoietin related | | | | |
| U05837 | 1.00 | −1.30 | 0.82 | 0.94 | 3.68 | 2.47 | 5.23 | 1.66 | 5.79 | 1.04 | hexosaminidase | U05837 | beta- | lysosomal enzymes | 9 29.0 cM | Proteolytic | | |
| | | | | | | | | | | | A; Hexa | | hexosaminidase | that contribute to the | | | | |
| | | | | | | | | | | | | | alpha chain | degradation of | | | | |
| | | | | | | | | | | | | | precursor (ec | glycoproteins, | | | | |
| | | | | | | | | | | | | | 3.2.1.52) | glycolipids, and | | | | |
| | | | | | | | | | | | | | (n-acetyl-beta- | glycosaminoglycans, | | | | |
| | | | | | | | | | | | | | glucosaminida | ganglioside | | | | |
| | | | | | | | | | | | | | (beta-n- | associated with | | | | |
| | | | | | | | | | | | | | acetylhexosamin- | ganglioside | | | | |
| | | | | | | | | | | | | | idase) (hexosa- | degradation. absence of | | | | |
| | | | | | | | | | | | | | minidase a). | the b form is | | | | |
| | | | | | | | | | | | | | | associated with | | | | |
| Msa.1600.0 | 1.00 | 1.12 | −0 54 | 1.14 | 0.56 | 2.11 | 4.88 | 0.13 | 5.61 | 0.65 | macrophage | L20315 | | transcripts from the | | EST; | | |
| | | | | | | | | | | | expressed gene 1; | | | gene are found at a | | Unknown | | |
| | | | | | | | | | | | Mpeg1 | | | high level in mature | | | | |
| | | | | | | | | | | | | | | macrophages and at a | | | | |
| | | | | | | | | | | | | | | moderate level in | | | | |
| | | | | | | | | | | | | | | certain | | | | |
| | | | | | | | | | | | | | | myelomonocytic cell | | | | |
| | | | | | | | | | | | | | | lines | | | | |
| L32974 | 1.00 | −2.05 | 1.79 | 0.94 | 0.01 | 1.10 | 1.71 | 0.31 | 5.57 | 1.47 | interferon- | L32974 | interferon- | | | | | |
| | | | | | | | | | | | induced | | induced | | | | | |
| | | | | | | | | | | | protein with | | protein with | | | | | |
| | | | | | | | | | | | tetratricopeptide | | tetratricopeptic | | | | | |
| | | | | | | | | | | | repeats 3; Ifit3 | | repeats 3 (ifit-2 | | | | | |
| | | | | | | | | | | | | | (glucocorticoid- | | | | | |
| | | | | | | | | | | | | | attenuated | | | | | |
| | | | | | | | | | | | | | response gene | | | | | |
| | | | | | | | | | | | | | 49 protein) | | | | | |
| | | | | | | | | | | | | | (garg-49)(irg2) | | | | | |
| AA387033 | 1.00 | 1.17 | 1.46 | 0.66 | 2.73 | 1.63 | 5.21 | 0.80 | 5.54 | 0.46 | Unknown | AA387033 | | | EST; | | | |
| | | | | | | | | | | | | | | | | Unknown | | |
| U72643 | 1.00 | 1.61 | 0.59 | 0.80 | 0.84 | 2.13 | 5.65 | 0.13 | 5.50 | 0.94 | leucocyte specific | U72643 | | defense/immunity | 17 19.06 | EST; | | |
| | | | | | | | | | | | transcript 1; Lst1 | | | protein. an integral | cM | Unknown | | |
| | | | | | | | | | | | | | | membrane protein. | | | | |
| AA174982 | 1.00 | −1.02 | 1.24 | 0.85 | 3.75 | 2.69 | 5.03 | 1.76 | 5.39 | 0.63 | coronin, actin | AA174982 | coronin-like | coronin participates in | 7 62.5 cM | Intracellular | | |
| | | | | | | | | | | | binding protein | | protein p57 | the remodelling of the | | Protein | | |
| | | | | | | | | | | | 1A; Corola | | (coronin 1a) | cortical actin | | | | |
| | | | | | | | | | | | | | (fragment). | cytoskeleton that is | | | | |
| | | | | | | | | | | | | | | responsible for | | | | |
| | | | | | | | | | | | | | | phagocytosis in | | | | |
| | | | | | | | | | | | | | | mammalian | | | | |
| | | | | | | | | | | | | | | neutrophils, a coronin- | | | | |
| | | | | | | | | | | | | | | like protein is also | | | | |
| | | | | | | | | | | | | | | associated with the | | | | |
| | | | | | | | | | | | | | | phenocytic apparatus | | | | |
| U54984 | 1.00 | −1.57 | 0.54 | 1.35 | 2.53 | 1.39 | 3.75 | 0.53 | 5.33 | 0.29 | matrix | U54984 | matrix | expressed in invasive | 14 12.5 | Proteolytic | | |
| | | | | | | | | | | | metalloproteinase | | metalloprotein- | lung carcinoma cells, | cM | | | |
| | | | | | | | | | | | 14 (membrane- | | ase-14 precursor | induces activation of | | | | |
| | | | | | | | | | | | inserted); Mmp14 | | (ec 3.4.24.—) | gelatinase a on the cell | | | | |
| | | | | | | | | | | | | | (mmp-14) | surface and enhances | | | | |
| | | | | | | | | | | | | | (membrane-type | cell invasion of | | | | |
| | | | | | | | | | | | | | matrix | basement membrane. | | | | |
| | | | | | | | | | | | | | metalloproteinase | specifically activates | | | | |
| | | | | | | | | | | | | | 1) (mt-mmp 1) | pro-gelatinase a. may | | | | |
| | | | | | | | | | | | | | (mtmmp1). | trigger invasion by | | | | |
| | | | | | | | | | | | | | | tumor cells by | | | | |
| | | | | | | | | | | | | | | activation | | | | |
| L38281 | 1.00 | −0.02 | 1.46 | 0.32 | 1.28 | 0.28 | 3.32 | 0.69 | 5.28 | 1.51 | immunorespon- | L38281 | immune- | | 14 53.5 | | | |
| | | | | | | | | | | | sive gene 1; Irg1 | | responsive | | cM | | | |
| | | | | | | | | | | | | | protein 1 | | | | | |
| AA245242 | 1.00 | −1.73 | 1.18 | 0.56 | 2.72 | 1.42 | 4.17 | 0.72 | 5.23 | 0.51 | MARCKS-like | AA245242 | lim domain | binds calmodulin and | 4 59.0 cM | Signal | | |
| | | | | | | | | | | | protein; M1p | | protein, cardiac | is a substrate for | | Transduction | | |
| | | | | | | | | | | | | | (muscle lim | protein kinase c | | | | |
| | | | | | | | | | | | | | protein) (cysteine- | | | | | |
| | | | | | | | | | | | | | rich protein 3) | | | | | |
| | | | | | | | | | | | | | (crp3).,marcks- | | | | | |
| | | | | | | | | | | | | | related protein | | | | | |
| | | | | | | | | | | | | | (mac-mareks) | | | | | |
| | | | | | | | | | | | | | (brain | | | | | |
| | | | | | | | | | | | | | protein f52) | | | | | |
| AA183642 | 1.00 | 1.24 | 1.21 | 0.76 | 0.87 | 2.08 | 9.10 | 1.34 | 5.10 | 0.86 | Unknown; EST | AA183642 | | | | EST; | | |
| | | | | | | | | | | | | | | | | Unknown | | |
| AA472322 | 1.00 | 1.00 | 3.10 | 0.52 | 2.32 | 1.32 | 6.68 | 0.28 | 5.09 | 1.40 | EST; Unknown | AA472322 | | | | EST; | | |
| | | | | | | | | | | | | | | | | Unknown | | |
| X16133 | 1.00 | −1.08 | −0.57 | 1.49 | −1.41 | 0.16 | 5.06 | 0.35 | 4.88 | 0.56 | proteoglycan, | X16133 | secretory granule | core protein for highly | | Intracellular | | |
| | | | | | | | | | | | secretory granule; | | proteoglycan core | acidic proteoglycan | | Protein | | |
| | | | | | | | | | | | Prg | | protein precursor | containing | | | | |
| | | | | | | | | | | | | | (mastocytoma | glycosaminoglycan | | | | |
| | | | | | | | | | | | | | proteoglycan core | that are almost | | | | |
| | | | | | | | | | | | | | protein) | exclusively | | | | |
| | | | | | | | | | | | | | (serglycin). | chondroitin sulfate e | | | | |
| Msa.15534.0 | 1.00 | −1.34 | 1.53 | 0.34 | 1.95 | 0.05 | 2.92 | 0.95 | 4.85 | 0.93 | neutrophil | W71124 | | | 1 76.1 cM | | | |
| | | | | | | | | | | | cytosolic | | | | | | | |
| | | | | | | | | | | | factor 2; Ncf2 | | | | | | | |
| Msa.22604.0 | 1.00 | −1.06 | −0.19 | 0.75 | 1.49 | 0.45 | 2.41 | 0.16 | 4.81 | 0.70 | | AA036297 | | | | | | |
| X61800 | 1.00 | 4.66 | 1.50 | 0.81 | 1.67 | 4.17 | 4.45 | 1.29 | 4.80 | 0.48 | CCAAT/enhancer | X61800 | ccaat/enhancer | a transcription factor | 16 9.0 cM | Transcription | | |
| | | | | | | | | | | | binding protein | | binding protein | that binds to cis- | | Factor | | |
| | | | | | | | | | | | (C/EBP), delta; | | delta (c/ebp delta) | regulatory dna | | | | |
| | | | | | | | | | | | Cebpd | | (c/ebp-related | sequences of viral | | | | |
| | | | | | | | | | | | | | protein 3). | genes and regulatory | | | | |
| | | | | | | | | | | | | | | sequences of cellular | | | | |
| | | | | | | | | | | | | | | genes that encode rna. | | | | |
| | | | | | | | | | | | | | | important | | | | |
| | | | | | | | | | | | | | | transcriptional | | | | |
| | | | | | | | | | | | | | | activator in the | | | | |
| | | | | | | | | | | | | | | regulation of genes | | | | |
| | | | | | | | | | | | | | | involved in immune | | | | |
| | | | | | | | | | | | | | | and inflammation | | | | |
| AA408170 | 1.00 | 2.81 | 3.78 | 0.59 | 3.24 | 1.50 | 5.10 | 0.95 | 4.80 | 1.06 | DEAD (aspartate- | AA408170 | | | Y 2.07 cM | EST; | | |
| | | | | | | | | | | | glutamate- | | | | | Unknown | | |
| | | | | | | | | | | | alanine- | | | | | | | |
| | | | | | | | | | | | aspartate) box | | | | | | | |
| | | | | | | | | | | | polypeptide, Y | | | | | | | |
| | | | | | | | | | | | chromosome: | | | | | | | |
| | | | | | | | | | | | Dbv | | | | | | | |
| C80550 | 1.00 | −2.09 | 0.70 | 0.65 | 1.34 | 0.18 | 4.20 | 0.44 | 4.78 | 1.27 | DNA segment, | C80550 | | seven transmembrane | | Cell Surface | | |
| | | | | | | | | | | | Cbr 13, Abbott 1 | | | domain protein, | | Protein | | |
| | | | | | | | | | | | expressed | | | upregulated during | | | | |
| | | | | | | | | | | | (TM7SF1) | | | kidney devewlopment, | | | | |
| X53247 | 1.00 | −1.36 | 0.91 | 0.62 | 0.75 | 1.80 | 3.63 | 0.14 | 4.77 | 0.85 | RAS-related C3 | X53247 | ras-related c3 | murine homolog of a | | Signal | | |
| | | | | | | | | | | | botulinum | | botulinum toxin | class of human ras- | | Transduction | | |
| | | | | | | | | | | | substrate 2; Rac2 | | substrate 2 (p21- | related proteins that | | | | |
| | | | | | | | | | | | | | rac2) (en-7 | are substrates for adp- | | | | |
| | | | | | | | | | | | | | protein | ribosylation by | | | | |
| | | | | | | | | | | | | | | botulinum toxin c3 | | | | |
| | | | | | | | | | | | | | | adp-ribosyltransferase. | | | | |
| | | | | | | | | | | | | | | expression is restricted | | | | |
| | | | | | | | | | | | | | | to hematopoietic cells | | | | |
| | | | | | | | | | | | | | | and organs. seems to | | | | |
| | | | | | | | | | | | | | | be involved in the | | | | |
| M32370 | 1.00 | −1.06 | 0.19 | 1.08 | 0.85 | 2.16 | 6.33 | 1.87 | 4.76 | 0.60 | SFFV proviral | M32370 | 31 kda trans- | encodes a tissue- | 2 47.5 cM | Transcription | | |
| | | | | | | | | | | | integration 1; | | forming protein | specific binding | | Factor | | |
| | | | | | | | | | | | Sfpi1 | | (transcription | protein, expressed in | | Factor | | |
| | | | | | | | | | | | | | factor pu 1). | macrophages and b | | | | |
| | | | | | | | | | | | | | | cells. media | | | | |
| | | | | | | | | | | | | | | conditioned by | | | | |
| | | | | | | | | | | | | | | erythroleukemia cells | | | | |
| | | | | | | | | | | | | | | expressing sfpi1 can | | | | |
| | | | | | | | | | | | | | | promote proliferation | | | | |
| | | | | | | | | | | | | | | of cell lines dependent | | | | |
| | | | | | | | | | | | | | | on mcsf or gm-csf, | | | | |
| | | | | | | | | | | | | | | may be inducing csf1 | | | | |
| Msa.23838.0 | 1.00 | −1.57 | −1.50 | 1.20 | 0.38 | 3.42 | 3.77 | 0.52 | 4.66 | 1.00 | tumor necrosis | AA051341 | | induced in vitro in | 2 | Regulatory | | |
| | | | | | | | | | | | factor induced | | | several cell types by | | | | |
| | | | | | | | | | | | protein 6; | | | proinflammatory | | | | |
| | | | | | | | | | | | Tnfip6 | | | cytokines, and in vivo | | | | |
| | | | | | | | | | | | | | | in pathological | | | | |
| | | | | | | | | | | | | | | conditions such as | | | | |
| | | | | | | | | | | | | | | rheumatoid arthritis | | | | |
| | | | | | | | | | | | | | | interacts with link | | | | |
| | | | | | | | | | | | | | | protein and aggrecan. | | | | |
| | | | | | | | | | | | | | | involved in matrix | | | | |
| | | | | | | | | | | | | | | dissociation and | | | | |
| | | | | | | | | | | | | | | regulated by ph | | | | |
| Msa.2530.0 | 1.00 | 1.00 | 1.30 | 0.30 | 1.23 | 0.23 | 5.83 | 1.26 | 4.66 | 1.14 | vav oncogene; | X64361 | vav | proable exchange | 17 32.7 | Signal | | |
| | | | | | | | | | | | Vav | | proto-oncogene. | factor for a small ras- | cM | Transduction | | |
| | | | | | | | | | | | | | | like gtp- binding | | | | |
| | | | | | | | | | | | | | | protein. can be | | | | |
| | | | | | | | | | | | | | | activated by truncation | | | | |
| | | | | | | | | | | | | | | of the n-terminus. | | | | |
| X07640 | 1.00 | −1.80 | 1.45 | 0.57 | 0.02 | 1.79 | 4.71 | 1.02 | 4.65 | 0.90 | integrin alpha M | X07640 | cell surface | mac-1 is a cell surface | | Cell Surface | | |
| | | | | | | | | | | | (Cd1 1b); Itgam | | glycoprotein mac- | glycoprotein of | | Protein | | |
| | | | | | | | | | | | | | 1 alpha subunit | monocytes, | | | | |
| | | | | | | | | | | | | | precursor (cr-3 | macrophages and | | | | |
| | | | | | | | | | | | | | alpha chain) | granulocytes which | | | | |
| | | | | | | | | | | | | | (cd11b) | has been implicated in | | | | |
| | | | | | | | | | | | | | (leukocyte | various adhesive | | | | |
| | | | | | | | | | | | | | adhesion receptor | interactions of these | | | | |
| | | | | | | | | | | | | | mol) (integrin | cells as well as in | | | | |
| | | | | | | | | | | | | | alpha m). | mediating the uptake | | | | |
| | | | | | | | | | | | | | | of complement-coated | | | | |
| | | | | | | | | | | | | | | particles. mac-1 is | | | | |
| | | | | | | | | | | | | | | identical with cr-3, the | | | | |
| | | | | | | | | | | | | | | receptor for the ic3b | | | | |
| | | | | | | | | | | | | | | fragment of the third | | | | |
| | | | | | | | | | | | | | | complement | | | | |
| | | | | | | | | | | | | | | component, mac-1 | | | | |
| | | | | | | | | | | | | | | probably recognize the | | | | |
| Msa.641.0 | 1.00 | −1.03 | −0.19 | 1.41 | 1.05 | 2.42 | 4.35 | 0.06 | 4.63 | 0.84 | Fe receptor, IgE, | W41745 | high affinity | the gamma subunit has | 1 93.3 cM | Receptor | | |
| | | | | | | | | | | | high affinity 1, | | immunoglobulin | a critical role in | | | | |
| | | | | | | | | | | | gamma poly- | | epsilon receptor | allowing the ige fc | | | | |
| | | | | | | | | | | | peptide; Fcer1g | | gamma-subunit | receptor to reach the | | | | |
| | | | | | | | | | | | | | precursor (fceri | cell surface | | | | |
| | | | | | | | | | | | | | (ige fc receptor, | | | | | |
| | | | | | | | | | | | | | gamma-subunit) | | | | | |
| | | | | | | | | | | | | | (fe-epsilon | | | | | |
| | | | | | | | | | | | | | ri-gamma) | | | | | |
| D10911 | 1.00 | −1.22 | 1.65 | 0.35 | 2.18 | 1.00 | 4.95 | 0.08 | 4.59 | 0.99 | a disinsegrin and | D10911 | adam 8 precursor | possible involvement | 7 F3-F5 | Proteolytic | | |
| | | | | | | | | | | | metalloprotease | | (ec 3.4.24.—) (a | in extravasation of | | | | |
| | | | | | | | | | | | domain (ADAM) | | disintegrin and | leukocytes. | | | | |
| | | | | | | | | | | | 8, Adam8 | | metalloproteinase | | | | | |
| | | | | | | | | | | | | | domain 8) (cell | | | | | |
| | | | | | | | | | | | | | surface antigen | | | | | |
| | | | | | | | | | | | | | ms2) | | | | | |
| | | | | | | | | | | | | | (macrophage | | | | | |
| | | | | | | | | | | | | | cysteine-rich | | | | | |
| | | | | | | | | | | | | | glycoprotein) | | | | | |
| | | | | | | | | | | | | | (cd156 antigen) | | | | | |
| X15591 | 1.00 | 1.53 | 0.39 | 0.89 | 0.63 | 1.86 | 3.57 | 0.27 | 4.58 | 0.64 | cytotoxic T | X15591 | ct1a-2-alpha | not known, expressed | 13 36.0 | Cell Surface | | |
| | | | | | | | | | | | lymphocyte- | | protein precursor. | in activated t-cell. | cM | Protein | | |
| | | | | | | | | | | | associated protein | | | | | | | |
| | | | | | | | | | | | 2 alpha: Ct1a2a | | | | | | | |
| AA285691 | 1.00 | 1.21 | 0.98 | 0.77 | 2.15 | 3.40 | 6.67 | 2.62 | 4.57 | 0.72 | cytohesin binding | AA285691 | | bone marrow derived | | Cell Surface | | |
| | | | | | | | | | | | protein (Cbp) | | | dendritic cells“” | | Protein | | |
| | | | | | | | | | | | | | | “”cloned by | | | | |
| | | | | | | | | | | | | | | subtraction of | | | | |
| | | | | | | | | | | | | | | activated bone marrow | | | | |
| | | | | | | | | | | | | | | macrophage versus | | | | |
| | | | | | | | | | | | | | | bone marrow-derived | | | | |
| | | | | | | | | | | | | | | dendritic cells“” | | | | |
| Msa.1529.0 | 1.00 | 1.00 | 0.58 | 0.47 | 0.44 | 0.64 | 2.46 | 0.31 | 4.50 | 0.60 | growth factor | U18996 | growth factor | plays a functional role | 11 8.0 cM | Signal | | |
| | | | | | | | | | | | receptor bound | | receptor-bound | in insulin and igf-i | | Transduction | | |
| | | | | | | | | | | | protein 10; Grb10 | | protein 10 (grb10 | signaling. may serve to | | Transduction | | |
| | | | | | | | | | | | | | adaptor protein). | positively link the | | | | |
| | | | | | | | | | | | | | | insulin and igf-i | | | | |
| | | | | | | | | | | | | | | receptors to an | | | | |
| | | | | | | | | | | | | | | uncharacterized | | | | |
| | | | | | | | | | | | | | | mitogenic signaling | | | | |
| | | | | | | | | | | | | | | pathway. interacts | | | | |
| | | | | | | | | | | | | | | with the cytoplasmic | | | | |
| | | | | | | | | | | | | | | domain of the | | | | |
| | | | | | | | | | | | | | | autophosphorylated | | | | |
| | | | | | | | | | | | | | | insulin receptor which | | | | |
| | | | | | | | | | | | | | | is then inhibited. the | | | | |
| | | | | | | | | | | | | | | interaction is mediated | | | | |
| | | | | | | | | | | | | | | by the sh2 domain, | | | | |
| | | | | | | | | | | | | | | also binds activated | | | | |
| | | | | | | | | | | | | | | platelet-derived | | | | |
| | | | | | | | | | | | | | | growth-factor receptor | | | | |
| | | | | | | | | | | | | | | and epidermal growth | | | | |
| X67783 | 1.00 | 2.51 | 0.70 | 0.76 | 0.80 | 1.94 | 2.37 | 0.17 | 4.49 | 0.49 | vascular cell | X67783 | vascular cell | cell-cell recognition. | 2 50.8 cM | Cell Surface | | |
| | | | | | | | | | | | adhesion | | adhesion protein | appears to function in | | Protein | | |
| | | | | | | | | | | | molecule 1; | | 1 precursor | leukocyte-endothelial | | | | |
| | | | | | | | | | | | Vcam1 | | (v-cam 1) | cell adhesion. interacts | | | | |
| | | | | | | | | | | | | | | with beta-1 integrin | | | | |
| | | | | | | | | | | | | | | v1a4 on leukocytes, | | | | |
| | | | | | | | | | | | | | | and mediates both | | | | |
| | | | | | | | | | | | | | | adhesion and signal | | | | |
| | | | | | | | | | | | | | | transduction. | | | | |
| | | | | | | | | | | | | | | vcam 1/v1a4 interaction | | | | |
| | | | | | | | | | | | | | | may play a | | | | |
| | | | | | | | | | | | | | | pathophysiologic role | | | | |
| X81582 | 1.00 | −1.60 | −0.48 | 1.27 | 0.82 | 2.27 | 2.74 | 0.12 | 4.48 | 0.38 | insulin-like | X81582 | insulin-like | binding proteins may | | Extracellular | | |
| | | | | | | | | | | | growth factor | | growth factor | act to distribute the | | Protein | | |
| | | | | | | | | | | | binding protein | | binding protein | 1gfs among the body | | | | |
| | | | | | | | | | | | 4; 1gfbp4 | | 4 precursor | fluid compartments, to | | | | |
| | | | | | | | | | | | | | (igfbp-4) (ibp-4) | protect the body from | | | | |
| | | | | | | | | | | | | | (igf binding | possible hypoglycemic | | | | |
| | | | | | | | | | | | | | protein 4). | effects of the igfs | | | | |
| Msa.2034.0 | 1.00 | −1.02 | 0.25 | 0.81 | 1.38 | 2.45 | 5.16 | 0.29 | 4.46 | 0.72 | CD53 antigen; | X97227 | leukocyte surface | may be involved in | 3 50.5 cM | Cell Surface | | |
| | | | | | | | | | | | Cd53 | | antigen cd53 (cell | growth regulation in | | Protein | | |
| | | | | | | | | | | | | | surface glyco- | hematopoietic cells. | | | | |
| | | | | | | | | | | | | | protein cd53). | | | | | |
| AA103744 | 1.00 | −1.31 | 1.73 | 1.20 | 3.98 | 2.12 | 7.13 | 2.86 | 4.42 | 0.81 | ribosomal protein | AA103744 | 60s ribosomal | play cardinal role in | 7 E2-F1 | Metabolic | | |
| | | | | | | | | | | | L27a; Rp127a | | protein 127a | calcium metabolism, | | | | |
| | | | | | | | | | | | | | (129). | and may be involved | | | | |
| | | | | | | | | | | | | | | in neural transmission. | | | | |
| | | | | | | | | | | | | | | buffers cytosolic | | | | |
| | | | | | | | | | | | | | | calcium. may | | | | |
| | | | | | | | | | | | | | | stimulate a membrane | | | | |
| | | | | | | | | | | | | | | ca(2+)-atpase and a | | | | |
| | | | | | | | | | | | | | | 3′,5′-cyclic nucleoside | | | | |
| | | | | | | | | | | | | | | phosphodiesterase. | | | | |
| | | | | | | | | | | | | | | expressed in many | | | | |
| | | | | | | | | | | | | | | tissues | | | | |
| AA189487 | 1.00 | 1.16 | 0.09 | 0.68 | 1.28 | 2.89 | 4.40 | 1.72 | 4.39 | 0.91 | sushi-repeat- | AA189487 | | | | EST; | | |
| | | | | | | | | | | | containing | | | | | Unknown | | |
| | | | | | | | | | | | protein, X | | | | | | | |
| | | | | | | | | | | | chromosome; | | | | | | | |
| | | | | | | | | | | | Srpx-pending | | | | | | | |
| Msa.1843.0 | 1.00 | −1.25 | 0.33 | 0.73 | 0.53 | 1.90 | 5.85 | 0.19 | 4.32 | 1.17 | chemokine(C—C) | U28404 | c—c chemokine | mip-1a-receptor, all | 9 72.0 cM | Receptor | | |
| | | | | | | | | | | | receptor 1, | | receptor type 1 | three of the cmkbr1 | | | | |
| | | | | | | | | | | | chemokine | | (c—c ckr-1) | genes has been found | | | | |
| | | | | | | | | | | | (C—C) receptor | | (cc-ckr-1) | in leukocytes, but their | | | | |
| | | | | | | | | | | | 1,-like | 2; | | (ccr-1) (ccr1) | patterns of expression | | | |
| | | | | | | | | | | | Cmkbr1, | | (macrophage | differ in solid organs. | | | | |
| | | | | | | | | | | | Cmkbr112 | | inflammatory | cmkbr1 is expressed in | | | | |
| | | | | | | | | | | | | | protein-1 alpha | heart, spleen, and | | | | |
| | | | | | | | | | | | | | receptor) (mip- | lung; | | | | |
| | | | | | | | | | | | | | 1alpha-r) (rantes- | | | | | |
| | | | | | | | | | | | | | r).,probable c—c | | | | | |
| | | | | | | | | | | | | | chemokine | | | | | |
| | | | | | | | | | | | | | receptor type | | | | | |
| | | | | | | | | | | | | | 3 (c—c ckr-3) | | | | | |
| | | | | | | | | | | | | | (cc-ckr-3) (ccr-3) | | | | | |
| | | | | | | | | | | | | | (ccr3) (ckr3) | | | | | |
| | | | | | | | | | | | | | (macrophage | | | | | |
| | | | | | | | | | | | | | inflammatory | | | | | |
| | | | | | | | | | | | | | protein-1 alpha | | | | | |
| | | | | | | | | | | | | | receptor-like 2) | | | | | |
| Msa.31660.0 | 1.00 | −1.04 | 0.86 | 0.72 | 0.81 | 2.03 | 4.38 | 0.48 | 4.28 | 0.73 | CD53 antigen; | AA105582 | leukocyte surface | may be involved in | 3 50.5 cM | Cell Surface | | |
| | | | | | | | | | | | Cd53 | | antigen cd53 (cell | growth regulation in | | Protein | | |
| | | | | | | | | | | | | | surface glyco- | hematopoietic cells. | | | | |
| | | | | | | | | | | | | | protein cd53). | | | | | |
| U29947 | 1.00 | −1.41 | −2.19 | 1.91 | 3.44 | 5.25 | 7.01 | 4.87 | 4.27 | 1.44 | mannosidasc 2, | U29947 | lysosomal alpha- | necessary for the | 8 37.0 cM | Regulatory | | |
| | | | | | | | | | | | alpha B1; | | mannosidase | catabolism of n-linked | | | | |
| | | | | | | | | | | | Man2b1 | | precursor (ec | carbohydrates released | | | | |
| | | | | | | | | | | | | | 3.2.1.24) | during glycoprotein | | | | |
| | | | | | | | | | | | | | (mannosidase, | turnover. cleaves all | | | | |
| | | | | | | | | | | | | | alpha b) | known types of alpha- | | | | |
| | | | | | | | | | | | | | (lysosomal acid | mannosidic linkages. | | | | |
| | | | | | | | | | | | | | alpha- | | | | | |
| | | | | | | | | | | | | | mannosidase) | | | | | |
| | | | | | | | | | | | | | (laman) | | | | | |
| AA546670 | 1.00 | −2.14 | 0.74 | 0.79 | 2.40 | 1.24 | 3.20 | 0.63 | 4.20 | 0.32 | myristoylated | AA546670 | myristoylated | marcks is the most | 10 22.0 | | | |
| | | | | | | | | | | | alanine rich | | alanine-rich c- | prominent cellular | cM | | | |
| | | | | | | | | | | | protein kinase C | | kinase substrate | substrate for protein | | | | |
| | | | | | | | | | | | substrate; Macs | | (marcks). | kinase c. this protein | | | | |
| | | | | | | | | | | | | | | binds calmodulm, | | | | |
| | | | | | | | | | | | | | | actin, and synapsin. | | | | |
| | | | | | | | | | | | | | | marcks is a | | | | |
| | | | | | | | | | | | | | | filamentous (f) actin | | | | |
| | | | | | | | | | | | | | | cross-linking protein | | | | |
| Msa.40979.0 | 1.00 | −1.35 | 2.97 | 0.90 | 1.29 | 2.51 | 3.55 | 1.17 | 4.19 | 1.07 | | AA161769 | | | | | | |
| AA544540 | 1.00 | −1.10 | 2.67 | 0.86 | 2.10 | 1.04 | 4.47 | 1.05 | 4.18 | 0.85 | | AA544540 | | | | | | |
| Msa.1099.0 | 1.00 | 1.13 | 0 92 | 0.61 | 2.24 | 0.74 | 3.25 | 0.27 | 4.15 | 0.40 | neuron specific | W46015 | neuron specific | | 5 21.0 cM | EST; | | |
| | | | | | | | | | | | gene family | | protein family | | | Unknown | | |
| | | | | | | | | | | | member 1; | | member 1 (brain | | | | | |
| | | | | | | | | | | | Nag1 | | neuron | | | | | |
| | | | | | | | | | | | | | cytoplasmic | | | | | |
| | | | | | | | | | | | | | protein 1) (p21) | | | | | |
| | | | | | | | | | | | | | (m234) | | | | | |
| U36993 | 1.00 | 1.03 | 0.47 | 0.64 | 0.33 | 1.42 | 4.96 | 1.07 | 4.12 | 0.97 | cytochrome P450, | U36993 | cytochromep450 | heme-containing | 3 1.0 cM | Metabolic | | |
| | | | | | | | | | | | 7b1; Cyp7b1 | | 7b1 (oxysterol 7- | enzymes involved in | | | | |
| | | | | | | | | | | | | | alpha- | metabolism of a | | | | |
| | | | | | | | | | | | | | hydroxylase) (ec | number of endogenous | | | | |
| | | | | | | | | | | | | | 1.14.13.—) | substrates. expressed | | | | |
| | | | | | | | | | | | | | (het-1). | principally in brain, | | | | |
| | | | | | | | | | | | | | | only low levels found | | | | |
| | | | | | | | | | | | | | | in liver. most closely | | | | |
| | | | | | | | | | | | | | | resembles p45viia1, | | | | |
| | | | | | | | | | | | | | | cholesterol 7 a | | | | |
| | | | | | | | | | | | | | | hydroxylase, but | | | | |
| | | | | | | | | | | | | | | clearly differs from it | | | | |
| AA170444 | 1.00 | 1.16 | 1 10 | 0.52 | 1.15 | 0.07 | 2.61 | 0.32 | 4.04 | 0.68 | EST; Unknown | AA170444 | | | | EST; | | |
| | | | | | | | | | | | | | | | | Unknown | | |
| AA289661 | 1.00 | −1.32 | 0 82 | 0.90 | 1.52 | 2.69 | 4.34 | 0.86 | 4.01 | 0.60 | EST; Unknown | AA289661 | | | | EST; | | |
| | | | | | | | | | | | | | | | | Unknown | | |
| K01496 | 1.00 | −1.66 | 0.56 | 0.76 | 1.77 | 3.25 | 4.14 | 1.13 | 4.01 | 0.40 | histocompatibility | K01496 | complement | factor b which is part | 17 18.85 | Hemostasis | | |
| | | | | | | | | | | | 2, complement | | factor b precursor | of the alternate | cM | | | |
| | | | | | | | | | | | component factor | | (ec 3.4.21.47) | pathway | | | | |
| | | | | | | | | | | | B; H2-Bf | | (c3/c5 | complement system is | | | | |
| | | | | | | | | | | | | | convertase). | cleaved by factor d | | | | |
| | | | | | | | | | | | | | | into 2 fragments: ba | | | | |
| | | | | | | | | | | | | | | and bb. bb, a serine | | | | |
| | | | | | | | | | | | | | | protease, then | | | | |
| | | | | | | | | | | | | | | combines with | | | | |
| | | | | | | | | | | | | | | complement factor 3b | | | | |
| | | | | | | | | | | | | | | to generate the c3 or | | | | |
| | | | | | | | | | | | | | | c5 convertase | | | | |
| AA189758 | 1.00 | −1.21 | 1.44 | 0.52 | 2.27 | 1.23 | 5.25 | 0.42 | 4.00 | 0.32 | Wbscr5 gene | AA189758 | | (see note) | | Regulatory | | |
| | | | | | | | | | | | product; Wbscr5 | | | two lim domains and a | | | | |
| | | | | | | | | | | | | | | putative protein kinase | | | | |
| | | | | | | | | | | | | | | domain, high | | | | |
| | | | | | | | | | | | | | | expression levels in | | | | |
| | | | | | | | | | | | | | | cns, particularly in | | | | |
| | | | | | | | | | | | | | | spinal cord, cranial | | | | |
| | | | | | | | | | | | | | | nerve and dorsal root | | | | |
| | | | | | | | | | | | | | | ganglia. lesser | | | | |
| | | | | | | | | | | | | | | espression in heart and | | | | |
| | | | | | | | | | | | | | | skeletal muscle. may | | | | |
| | | | | | | | | | | | | | | be a component of | | | | |
| U88328 | 1.00 | 1.73 | 0.18 | 0.87 | 1.95 | 4.32 | 5.37 | 1.50 | 4.00 | 0.28 | cytokine | U88328 | cytokine | socs-1 inhibits the | | ECM | | |
| | | | | | | | | | | | inducible | | inducible | macrophage | | (Matrix | | |
| | | | | | | | | | | | SH2-containing | | sh2-containing | differentiation of m1 | | Prot) | | |
| | | | | | | | | | | | protein 3 | | protein 3 (protein | cells in response to i1- | | | | |
| | | | | | | | | | | | (SOCS3); Cish3 | | ef-10). | 6. transcription of all | | | | |
| | | | | | | | | | | | | | | four socs genes is | | | | |
| | | | | | | | | | | | | | | increased rapidly in | | | | |
| | | | | | | | | | | | | | | response to i1-6, m | | | | |
| | | | | | | | | | | | | | | vitro and in vivo, | | | | |
| | | | | | | | | | | | | | | suggesting they may | | | | |
| | | | | | | | | | | | | | | act in a classic | | | | |
| | | | | | | | | | | | | | | position feedback | | | | |
| AA015322 | 1.00 | 1.70 | −1.39 | 0.77 | −0.73 | 2.09 | 2.86 | 2.87 | 3 98 | 1.02 | SPARC-related | AA015322 | | | | Other | | |
| | | | | | | | | | | | protein (SRG) | | | | | | | |
| | | | | | | | | | | | (related to | | | | | | | |
| | | | | | | | | | | | isteonectin) | | | | | | | |
| M13963 | 1 00 | −1.39 | 2.19 | 0.75 | 1.70 | 0.25 | 3.64 | 1.35 | 3.92 | 0.80 | guanine | M13963 | guanine | the g(1) proteins are | 9 59.0 cM | Signal | | |
| | | | | | | | | | | | nucleolide bind- | | nucleotide-bind- | involved in hormonal | | Transduction | | |
| | | | | | | | | | | | ing protein, alpha | | ing protein g(i), | regulation of adenylate | | | | |
| | | | | | | | | | | | inhibiting 2; | | alpha-2 subunit | cyclase; they inhibit | | | | |
| | | | | | | | | | | | Gnai2 | | (adenylate | the cyclase in response | | | | |
| | | | | | | | | | | | | | cyclase-inhibiting | to beta- adrenergic | | | | |
| | | | | | | | | | | | | | g alpha protein). | stimuli | | | | |
| Z16078 | 1.00 | 1.06 | 0.06 | 1.04 | 0.83 | 2.18 | 3.64 | 0.16 | 3.92 | 0.55 | CD53 antigen; | Z16078 | leukocyte surface | may be involved in | 3 50.5 cM | Cell Surface | | |
| | | | | | | | | | | | Cd53 | | antigen cd53 (cell | growth regulation in | | Protein | | |
| | | | | | | | | | | | | | surface glyco- | hematopoietic cells. | | | | |
| | | | | | | | | | | | | | protein cd53). | | | | | |
| M63836 | 1.00 | −1.50 | −0.08 | 0.81 | 0.65 | 2.02 | 3.88 | 0.75 | 3.87 | 0.12 | beta- | M63836 | beta- | aka gusb | 5 72.0 cM | Metabolic | | |
| | | | | | | | | | | | glucuronidase | | glucuronidase | | | | | |
| | | | | | | | | | | | structural; Gus-s | | precursor (ec | | | | | |
| | | | | | | | | | | | | | 3.2.1.31). | | | | | |
| D11468 | 1.00 | 1.82 | 1.20 | 0.78 | 2.26 | 0.93 | 0.95 | 1.00 | 3.84 | 1.15 | immunoglobulin | D11468 | | | | Extracellular | | |
| | | | | | | | | | | | alpha heavy chain | | | | | Protein | | |
| AA266385 | 1.00 | −1.03 | 1.00 | 0.58 | 1.12 | 0.04 | 3.88 | 0.46 | 3.84 | 0.84 | DNA segment, | AA266385 | | | X 1.6 cM | EST; | | |
| | | | | | | | | | | | Chr X, | | | | | Unknown | | |
| | | | | | | | | | | | Immmunex 39, | | | | | | | |
| | | | | | | | | | | | expressed; | | | | | | | |
| U29762 | 1.00 | 3.11 | 2.20 | 1.45 | 5.23 | 2.30 | 3.07 | 1.90 | 3.83 | 1.07 | D site albumin | U29762 | d-site-binding | this transcriptional | 7 23.0 cM | Transcription | | |
| | | | | | | | | | | | promoter binding | | protein (albumin | activator recognizes | | Factor | | |
| | | | | | | | | | | | protein; Dbp | | d box-binding | and binds to the | | | | |
| | | | | | | | | | | | | | protein). | sequence 5′-rttaygtaay- | | | | |
| | | | | | | | | | | | | | | 3′ found in the | | | | |
| | | | | | | | | | | | | | | promotor of genes | | | | |
| | | | | | | | | | | | | | | such as albumin, | | | | |
| | | | | | | | | | | | | | | cyp2a4 and cyp2a5, it | | | | |
| | | | | | | | | | | | | | | is not essential for | | | | |
| | | | | | | | | | | | | | | circadian rhythm | | | | |
| | | | | | | | | | | | | | | generation, but | | | | |
| | | | | | | | | | | | | | | modulates important | | | | |
| | | | | | | | | | | | | | | clock output genes. | | | | |
| | | | | | | | | | | | | | | may be a direct target | | | | |
| | | | | | | | | | | | | | | for regulation by the | | | | |
| | | | | | | | | | | | | | | circadian pacemaker | | | | |
| | | | | | | | | | | | | | | component clock. may | | | | |
| | | | | | | | | | | | | | | affect circadian period | | | | |
| AA177433 | 1.00 | 1.34 | 0.21 | 0.82 | 0.49 | 2.05 | 4.56 | 0.86 | 3.82 | 0.51 | EST; Unknown | AA177433 | | | | EST; | | |
| | | | | | | | | | | | | | | | | Unknown | | |
| D28599 | 1.00 | −1.28 | 0.91 | 0.59 | 1.18 | 0.13 | 4.59 | 0.54 | 3.81 | 0.96 | chondroitin | D28599 | versican core | extracellular matrix | 13 55.0 | ECM | | |
| | | | | | | | | | | | sulfate | | protein precursor | link protein. | cM | (Matrix | | |
| | | | | | | | | | | | proteoglycan 2; | | (large fibroblast | | | Prot) | | |
| | | | | | | | | | | | Cspg2 | | proteoglycan) | | | | | |
| | | | | | | | | | | | | | (chondroitin | | | | | |
| | | | | | | | | | | | | | sulfate | | | | | |
| | | | | | | | | | | | | | proteoglycan core | | | | | |
| | | | | | | | | | | | | | protein 2)(ng-m) | | | | | |
| D50494 | 1.00 | −2.14 | 11.79 | 6.74 | 3.21 | 2.21 | 4.11 | 1.50 | 3.78 | 0.92 | DEAD(aapartate- | D50494 | probable atp- | 9 26.0 cM | | | | |
| | | | | | | | | | | | glutamate- | | dependent rna | | | | | |
| | | | | | | | | | | | alanine-aspartate) | helicase p54 | | | | | | |
| | | | | | | | | | | | box polypeptide | | (oncogene rck | | | | | |
| | | | | | | | | | | | 6; Ddx6 | | homolog) (dead- | | | | | |
| | | | | | | | | | | | | | box protein 6) | | | | | |
| L15443 | 1.00 | 1.18 | 0.10 | 1.49 | 2.42 | 0.90 | 3.03 | 0.39 | 3.78 | 0.42 | membrane | L15443 | | | | Cell Surface | | |
| | | | | | | | | | | | component, | | | | | Protein | | |
| | | | | | | | | | | | surface marker | | | | | | | |
| | | | | | | | | | | | 1: M3s1 | | | | | | | |
| ET62103 | 1.00 | −1.46 | 10.80 | 4.19 | 6.12 | 8.71 | 6.88 | 5.42 | 3.78 | 2.72 | | ET62103 | | | | | | |
| U88908 | 1.00 | 1.55 | 1.04 | 0.58 | 1.90 | 0.66 | 5.13 | 1.33 | 3.77 | 0.55 | apoptosia | U88908 | inhibitor of | traf1 and/or traf2 | 9 A2 | Regulatory | | |
| | | | | | | | | | | | inhibitor 1; | | apoptosis protein | associated protein of | | | | |
| | | | | | | | | | | | birc2 (Api1) | | 1 (miap1) | the iap (inhibitor of | | | | |
| | | | | | | | | | | | | | (miap-1) | apoptosis) family. iaps | | | | |
| | | | | | | | | | | | | | | may play a role in | | | | |
| | | | | | | | | | | | | | | tumour progression | | | | |
| | | | | | | | | | | | | | | rather than tumour | | | | |
| | | | | | | | | | | | | | | initiation, making the | | | | |
| | | | | | | | | | | | | | | iaps an attractive | | | | |
| | | | | | | | | | | | | | | therapeutic target | | | | |
| AA020512 | 1.00 | 1.01 | 1.31 | 0.45 | 1.33 | 0.02 | 3.97 | 0.39 | 3.75 | 0.87 | caspase 6; Casp6 | AA020512 | caspase-6 | involved in the | | | | |
| | | | | | | | | | | | | | precursor | activation cascade of | | | | |
| | | | | | | | | | | | | | (cc 3.4.22.—) | caspases responsible | | | | |
| | | | | | | | | | | | | | (apoptotic pro- | for apoptosis | | | | |
| | | | | | | | | | | | | | tease mch-2). | execution. cleaves | | | | |
| | | | | | | | | | | | | | | poly(adp-ribose) | | | | |
| | | | | | | | | | | | | | | polymerase in vitro, as | | | | |
| | | | | | | | | | | | | | | well as lamins. | | | | |
| | | | | | | | | | | | | | | overexpression | | | | |
| | | | | | | | | | | | | | | promotes programmed | | | | |
| | | | | | | | | | | | | | | cell death (by | | | | |
| | | | | | | | | | | | | | | similarity) | | | | |
| AA172851 | 1.00 | −1.04 | 0 26 | 0.77 | 0.44 | 1.66 | 4.53 | 0.34 | 3.75 | 0.58 | EST; Unknown | AA172851 | | no match on blast | | EST; | | |
| | | | | | | | | | | | | | | | | Unknown | | |
| X75926 | 1.00 | 2.26 | 0.55 | 0.56 | 1.93 | 0.81 | 2.51 | 0.07 | 3.74 | 0.54 | ATP-binding | X75926 | atp-binding | camp-dependent and | 4 23.1 cM | | | |
| | | | | | | | | | | | cassette, sub- | | cassette, sub- | sulfonylurea-sensitive | | | | |
| | | | | | | | | | | | family A (ABC1), | | family a, | anion transporter. key | | | | |
| | | | | | | | | | | | member 1; Abca1 | | member 1 (atp- | gatekeeper influencing | | | | |
| | | | | | | | | | | | | | binding cassette | intracellular | | | | |
| | | | | | | | | | | | | | transporter 1) | cholesterol transport | | | | |
| | | | | | | | | | | | | | (atp-binding | (by similarity) | | | | |
| | | | | | | | | | | | | | cassette 1) (abc-1) | | | | | |
| Msa.22134.0 | 1.00 | −1.17 | −0.52 | 1.02 | 2.09 | 0.87 | 3.68 | 0.40 | 3.72 | 0.48 | Unknown | AA031158 | | | EST; | | | |
| | | | | | | | | | | | | | | | | Unknown | | |
| U59463 | 1.00 | 1.12 | 1.47 | 0.50 | 1.56 | 0.11 | 3.86 | 0.75 | 3.72 | 0.86 | caspase 11; | U59463 | caspase-11 | involved in the | | | | |
| | | | | | | | | | | | Casp11 | | precursor (ec | activation cascade of | | | | |
| | | | | | | | | | | | | | 3.4.22.) (ich-3 | cascapases responsible | | | | |
| | | | | | | | | | | | | | protease). | for apoptosis | | | | |
| | | | | | | | | | | | | | | execution. promotes i1- | | | | |
| | | | | | | | | | | | | | | 1 beta processing by | | | | |
| | | | | | | | | | | | | | | ice, so may also have a | | | | |
| | | | | | | | | | | | | | | role in inflammatory | | | | |
| | | | | | | | | | | | | | | responses | | | | |
| Msa.510.0 | 1.00 | −2.49 | −0.90 | 1.04 | 0.25 | 1.81 | 2.97 | 0.16 | 3.71 | 0.50 | histocompatibility | M57890 | complement | involved in the | 17 18.85 | Hemostasis | | |
| | | | | | | | | | | | 2, complement | | factor b precursor | alternative or | cM | | | |
| | | | | | | | | | | | component factor | | (ec 3.4.21.47) | properdin complement | | | | |
| | | | | | | | | | | | B; H2-Bf | | (c3/c5 | pathway | | | | |
| | | | | | | | | | | | | | convertase). | | | | | |
| AA172673 | 1.00 | 1.32 | 1.17 | 0.44 | 1.53 | 0.29 | 3.48 | 0.41 | 3.70 | 0.63 | paternally | AA172673 | | | 7 6.5 cM | EST; | | |
| | | | | | | | | | | | expressed | | | | | Unknown | | |
| | | | | | | | | | | | gene 3; Peg3 | | | | | | | |
| AA051505 | 1.00 | −2.70 | −1.31 | 0.66 | 0.87 | 2.64 | 3.03 | 0.83 | 3.70 | 0.72 | EST; unknown | AA051505 | | | | EST; | | |
| | | | | | | | | | | | | | | | | Unknown | | |
| U89269 | 1.00 | −1.43 | 0.80 | 0.75 | 2.05 | 3.22 | 4.88 | 1.64 | 3.69 | 0.60 | cathepsin C; Ctsc | U89269 | dipeptidyl- | mammalian lysosomal | 7 D3-E1.1 | Proteolytic | | |
| | | | | | | | | | | | | | peptidase i | cysteine proteinascs. | | | | |
| | | | | | | | | | | | | | precursor (ec | plays a role in protein | | | | |
| | | | | | | | | | | | | | 3.4.14.1) (dpp-i) | catabolism within the | | | | |
| | | | | | | | | | | | | | (dppi) (cathepsin | cell, and may be | | | | |
| | | | | | | | | | | | | | c) (cathepsin j) | involved in tumor | | | | |
| | | | | | | | | | | | | | (dipeptidyl | metastasis. expression | | | | |
| | | | | | | | | | | | | | transferase). | is widely distributed, | | | | |
| | | | | | | | | | | | | | | with some variability | | | | |
| | | | | | | | | | | | | | | in level, m mouse | | | | |
| | | | | | | | | | | | | | | tissues | | | | |
| J03535 | 1.00 | −1.00 | 0.79 | 0.72 | 2.43 | 1.30 | 3.93 | 1.01 | 3.68 | 0.20 | embigin; Emb | 303535 | pou domain, class | embigin and basigin | 15 57.0 | Transcription | | |
| | | | | | | | | | | | | | 6, transcnption | are highly | | Factor | | |
| | | | | | | | | | | | | | factor 1 (octamer- | glycossylated | | | | |
| | | | | | | | | | | | | | binding trans- | transmembrane | | | | |
| | | | | | | | | | | | | | cription factor | glycoproteins with two | | | | |
| | | | | | | | | | | | | | emb) (transcrip- | immunoglobulin | | | | |
| | | | | | | | | | | | | | tion regulatory | domains and form a | | | | |
| | | | | | | | | | | | | | protein mcp-1)., | subgroup in the | | | | |
| | | | | | | | | | | | | | teratocarcinonsa | immunoglubulin | | | | |
| | | | | | | | | | | | | | glycoprotein | superfamily. embigin | | | | |
| | | | | | | | | | | | | | gp-70 precursor. | is strongly expressed | | | | |
| | | | | | | | | | | | | | | in the endoderm | | | | |
| | | | | | | | | | | | | | | during | | | | |
| AA592768 | 1.00 | 1 54 | 2.46 | 0.83 | 4.64 | 1.76 | 5.53 | 2.15 | 3.66 | 0.49 | small proline-rich | AA592768 | | small proline-rich | 3 45.2 cM | Structural | | |
| | | | | | | | | | | | protein 2A; | | | (spr) proteins are | | Protein | | |
| | | | | | | | | | | | Sprr2a | | | structural components | | | | |
| | | | | | | | | | | | | | | of the cornified cell | | | | |
| | | | | | | | | | | | | | | envelop of stratified | | | | |
| | | | | | | | | | | | | | | aquamous epithelia, | | | | |
| | | | | | | | | | | | | | | they are subdivided | | | | |
| | | | | | | | | | | | | | | into three families, | | | | |
| | | | | | | | | | | | | | | i.e., spr1, spr2, and | | | | |
| | | | | | | | | | | | | | | spr3, of which the spr2 | | | | |
| | | | | | | | | | | | | | | family is the most | | | | |
| AA259937 | 1.00 | −5.79 | −1.14 | 0.85 | 0.60 | 2.69 | 2.67 | 0.61 | 3.65 | 0.51 | procollagen, type | AA259937 | collagen alpha | collagen type iii | 1 21.1 cM | ECM | | |
| | | | | | | | | | | | III, alpha 1; | | 1(in) chain | occurs in most soft | | (Matrix | | |
| | | | | | | | | | | | Col3a1 | | precursor. | connective tissues | | Prot) | | |
| | | | | | | | | | | | | | | along with type i | | | | |
| M31419 | 1.00 | 1.09 | 1.63 | 0.64 | 1.42 | 0.20 | 4.04 | 0.65 | 3.65 | 1.09 | interferon | M31419 | interferon- | member of a cluster of | 1 95.2 cM | Unknown | | |
| | | | | | | | | | | | activated gene | | activatable | genes activated by | | | | |
| | | | | | | | | | | | 204; 1fi204 | | protein 204 (ifi- | interferon on chr 1. | | | | |
| | | | | | | | | | | | | | 204) (interferon- | function unknown. | | | | |
| | | | | | | | | | | | | | inducible protein | | | | | |
| X13333 | 1.00 | −1.22 | −0.21 | 0.70 | 0.54 | 1.83 | 3.09 | 0.31 | 3.65 | 0.66 | CD14 antigen; | X13333 | monocyte | a cell surface marker | 18 31.0 | Cell Surface | | |
| | | | | | | | | | | | Cd14 | | differentiation | of human monocytes | cM | Protein | | |
| | | | | | | | | | | | | | antigen cd14 | and macrophages. | | | | |
| | | | | | | | | | | | | | precursor (1ps | serves aa an 1ps | | | | |
| | | | | | | | | | | | | | receptor) (1ps-r) | receptor controlling | | | | |
| | | | | | | | | | | | | | (myeloid cell- | cell activation under | | | | |
| | | | | | | | | | | | | | specific | physiological | | | | |
| | | | | | | | | | | | | | leucine-rich | conditions. when 1ps | | | | |
| | | | | | | | | | | | | | glycoprotein). | binds to cd14 the cells | | | | |
| | | | | | | | | | | | | | | become activated and | | | | |
| | | | | | | | | | | | | | | release cytokines and | | | | |
| | | | | | | | | | | | | | | unregulated cell | | | | |
| Msa.17760.0 | 1.00 | 1.18 | −0.09 | 0.59 | 1.30 | 0.26 | 2.25 | 0.17 | 3.65 | 0.46 | | W98059 | | | | | | |
| Msa.10687.0 | 1.00 | −7.85 | −1.32 | 1.27 | 0.53 | 1.55 | 2.43 | 0.50 | 3.62 | 0.29 | Unknown | W48936 | | | EST; | | | |
| | | | | | | | | | | | | | | | | Unknown | | |
| D16262 | 1.00 | −2.89 | −2.10 | 0.78 | −0.63 | 1.88 | 1.84 | 0.27 | 3.59 | 0.99 | mesoderm | D16262 | | aka ppeg-1 -- the mouse | 6 7.5 cM | Cytokine | | |
| | | | | | | | | | | | specific | | | peg1/mest gene is an | | | | |
| | | | | | | | | | | | transcript; Mest | | | imprinted gene that is | | | | |
| | | | | | | | | | | | | | | expressed particularly | | | | |
| | | | | | | | | | | | | | | in mesodermal tissues | | | | |
| | | | | | | | | | | | | | | in early embryonic | | | | |
| | | | | | | | | | | | | | | stages | | | | |
| M31131 | 1.00 | −1.13 | 0.01 | 0.80 | 0.44 | 1.57 | 2.15 | 0.39 | 3.58 | 0.51 | cadherin 2; Cdh2 | M31131 | neural-cadherin | cadherins are calcium | 18 6.0 cM | Cell Surface | | |
| | | | | | | | | | | | | | precursor (n- | dependent cell | | | | |
| | | | | | | | | | | | | | cadherin). | adhesion proteins. | | | | |
| | | | | | | | | | | | | | | they prefernetially | | | | |
| | | | | | | | | | | | | | | interact with | | | | |
| | | | | | | | | | | | | | | themselves in a | | | | |
| | | | | | | | | | | | | | | homophilic manner m | | | | |
| | | | | | | | | | | | | | | connecting cells; | | | | |
| | | | | | | | | | | | | | | cadherins may thus | | | | |
| | | | | | | | | | | | | | | contribute to the | | | | |
| | | | | | | | | | | | | | | sorting of | | | | |
| | | | | | | | | | | | | | | heterogeneous cell | | | | |
| | | | | | | | | | | | | | | types, n-cadherin may | | | | |
| | | | | | | | | | | | | | | be involved in | | | | |
| AA161790 | 1.00 | −1.89 | 2.16 | 0.67 | 2.68 | 0.16 | 3.54 | 0.34 | 3.56 | 0.25 | splysia ras-related | AA161790 | | regulates a signal | 2 37.0 cM | Intracellular | | |
| | | | | | | | | | | | homolog A2; | | | transduction pathway | | Protein | | |
| | | | | | | | | | | | Arha2 | | | linking plasma | | | | |
| | | | | | | | | | | | | | | membrane receptors to | | | | |
| | | | | | | | | | | | | | | the assembly of focal | | | | |
| | | | | | | | | | | | | | | adhesions and actin | | | | |
| | | | | | | | | | | | | | | stress fibers | | | | |
| D83266 | 1.00 | −1.47 | 0.12 | 0.69 | 0.47 | 1.66 | 4.05 | 0.84 | 3.55 | 0.78 | vav oncogene; | D83266 | vav proto- | binds to grb2 and grb3- | 17 32.7 | Signal | | |
| | | | | | | | | | | | Vav | | oncogene. | 3 | cM | Transduction | | |
| M35833 | 1.00 | −1.98 | −1.06 | 0.88 | 0.24 | 1.52 | 0.75 | 0.95 | 3.54 | 0.48 | midkine; Mdk | M35833 | midkine precursor | midkine (mk) is a | 2 53.0 cM | Cytokine | | |
| | | | | | | | | | | | | | (retinoic acid- | heparin-binding | | | | |
| | | | | | | | | | | | | | induced | growth/differentiation | | | | |
| | | | | | | | | | | | | | differentiation | factor implicated in | | | | |
| | | | | | | | | | | | | | factor). | the control of | | | | |
| | | | | | | | | | | | | | | development and | | | | |
| | | | | | | | | | | | | | | repair of various | | | | |
| | | | | | | | | | | | | | | tissues. mk plays | | | | |
| | | | | | | | | | | | | | | important roles in | | | | |
| | | | | | | | | | | | | | | chondrogenesis and | | | | |
| | | | | | | | | | | | | | | contributor to bone | | | | |
| L23801 | 1.00 | 4.89 | −2.21 | 1.27 | 0.70 | 2.36 | 2.59 | 0.70 | 3.54 | 0.51 | integrin binding | L23801 | bone sialoprotein | aka integrin binding | 5 56.0 cM | ECM | | |
| | | | | | | | | | | | sialoprotein; 1bsp | | ii precursor (bsp | sialoprotein -- bone | | (Matrix | | |
| | | | | | | | | | | | | | ii) (cell-binding | sialoprotein (bsp) is a | | Prot) | | |
| | | | | | | | | | | | | | sialoprotein). | small, highly | | | | |
| | | | | | | | | | | | | | (integrin-binding | posttranslationally | | | | |
| | | | | | | | | | | | | | sialoprotein). | modified integrin | | | | |
| | | | | | | | | | | | | | | binding protein found | | | | |
| | | | | | | | | | | | | | | in the mineral | | | | |
| | | | | | | | | | | | | | | compartment of | | | | |
| | | | | | | | | | | | | | | developing bone | | | | |
| | | | | | | | | | | | | | | contains a conserved | | | | |
| | | | | | | | | | | | | | | arg-gly-asp(rgd) | | | | |
| | | | | | | | | | | | | | | intensic binding | | | | |
| U78818 | 1.00 | −2.25 | 1.14 | 0.45 | 1.09 | 0.02 | 3.63 | 0.37 | 3.54 | 0.45 | downstream of | U78818 | | | 6 34.73 | | | |
| | | | | | | | | | | | tyrosine kinase 1; | | | | cM | | | |
| | | | | | | | | | | | Dok1 | | | | | | | |
| U19482 | 1.00 | −1.31 | 1.07 | 0.85 | 1.65 | 2.68 | 6.02 | 0.87 | 3.52 | 0.32 | small inducible | U19482 | small inducible | a.k.a. mip-1 gamma or | 11 47.4 | Cytokine | | |
| | | | | | | | | | | | cytokine A9; | | cytokine a9 | ccf18 -- chemokines | cM | | | |
| | | | | | | | | | | | Scya9 | | precursor | play an important role | | | | |
| | | | | | | | | | | | | | (macrophage | in immune and | | | | |
| | | | | | | | | | | | | | inflammatory | inflammatory | | | | |
| | | | | | | | | | | | | | protein 1-gamma) | responses by inducing | | | | |
| | | | | | | | | | | | | | (mip-1-gamma) | migration and | | | | |
| | | | | | | | | | | | | | (macrophage | adhesion of | | | | |
| | | | | | | | | | | | | | inflammatory | leukocytes. ccf18 | | | | |
| | | | | | | | | | | | | | protein-related | mma is constitutively | | | | |
| | | | | | | | | | | | | | protein-2) (mrp-2) | expressed in | | | | |
| | | | | | | | | | | | | | (ccf18). | macrophage and | | | | |
| | | | | | | | | | | | | | | myeloid cell lines and | | | | |
| U21795 | 1.00 | −1.16 | 1.05 | 0.42 | 1.53 | 0.36 | 4.01 | 0.56 | 3.51 | 0.61 | interleukin 2 | U21795 | cytokine receptor | common subunit for | X 38.0 cM | | | |
| | | | | | | | | | | | receptor, gamma | | common gamma | the receptors for a | | | | |
| | | | | | | | | | | | chain; I12rg | | chain precursor | variety of interleukins. | | | | |
| | | | | | | | | | | | | | (gamma-c) | | | | | |
| | | | | | | | | | | | | | (interleukin-2 | | | | | |
| | | | | | | | | | | | | | receptor gamma | | | | | |
| | | | | | | | | | | | | | chain) (i1-2r | | | | | |
| | | | | | | | | | | | | | gamma chain) | | | | | |
| | | | | | | | | | | | | | (p64) | | | | | |
| M27960 | 1.00 | −1.14 | −0.88 | 1.09 | 1.29 | 2.91 | 5.45 | 0.51 | 3.49 | 0.49 | interleukin 4 | M27960 | interleukin-4 | a receptor for i14, a | 7 62.0 cM | Receptor | | |
| | | | | | | | | | | | receptor, alpha; | | receptor alpha | mediator of the th2 (b | | | | |
| | | | | | | | | | | | I14ra | | chain precursor | cell) response. acts as | | | | |
| | | | | | | | | | | | | | (i1-4r-alpha). | an antagonist to i14, | | | | |
| | | | | | | | | | | | | | | presumably by | | | | |
| | | | | | | | | | | | | | | absorbing i14 | | | | |
| | | | | | | | | | | | | | | molecules | | | | |
| W11156 | 1.00 | 0.01 | 0.61 | 1.10 | 1.64 | 0.10 | 3.19 | 0.05 | 3.49 | 0.67 | EST; unknown | W11156 | | some similarity to | | EST; | | |
| | | | | | | | | | | | | | | j399 human gamma- | | Unknown | | |
| | | | | | | | | | | | | | | interferon-inducible | | | | |
| | | | | | | | | | | | | | | protein | | | | |
| D67076 | 1.00 | −1.17 | 0.46 | 0.89 | 0.18 | 1.63 | 3.51 | 0.21 | 3.48 | 0.83 | a disintegrin-like | D67076 | adam-ts 1 | expression is markedly | 16 53.4 | Proteolytic | | |
| | | | | | | | | | | | and | | precursor (ec | and selectively | cM | | | |
| | | | | | | | | | | | metalloprolease | | 3.4.24.—) (a | induced by | | | | |
| | | | | | | | | | | | (reprolysin type) | | disintegrin and | lipopolysaccharide | | | | |
| | | | | | | | | | | | with thrombo- | | metalloproteinase | administration in the | | | | |
| | | | | | | | | | | | spondin type 1 | | with | kidney and heart. | | | | |
| | | | | | | | | | | | motif, 1; Adamts1 | | thrombospondin | developmentally | | | | |
| | | | | | | | | | | | | | motifs 1) | regulated membrane | | | | |
| | | | | | | | | | | | | | (adamts-1) | proteins containing | | | | |
| | | | | | | | | | | | | | (adam-ts1). | disintegrin and | | | | |
| | | | | | | | | | | | | | | metalloproteinase | | | | |
| AA185911 | 1.00 | −1.23 | 0.41 | 0.65 | 1.10 | 2.48 | 3.56 | 0.86 | 3.47 | 0.60 | lymphocyte | AA185911 | | lymphocyte antigen. | | Cell Surface | | |
| | | | | | | | | | | | antigen 68; Ly68 | | | | | Protein | | |
| X84797 | 1.00 | −1.58 | 0.52 | 0.66 | 0.27 | 1.63 | 3.78 | 0.17 | 3.46 | 0.43 | hematopoictic | X84797 | hematopoietic | substrate of the | 16 B | Hemostasis | | |
| | | | | | | | | | | | cell specific Lyn | | lineage cell | antigen receptor- | | | | |
| | | | | | | | | | | | substrate 1; Hcls1 | | specific protein | coupled tyrosine | | | | |
| | | | | | | | | | | | | | (hematopoietic | kinase. plays a role in | | | | |
| | | | | | | | | | | | | | cell-specific lyn | antigen receptor | | | | |
| | | | | | | | | | | | | | substrate 1) | signaling for both | | | | |
| | | | | | | | | | | | | | (lckbp1). | clonal expansion and | | | | |
| | | | | | | | | | | | | | | deletion m lymphoid | | | | |
| | | | | | | | | | | | | | | cells directly | | | | |
| | | | | | | | | | | | | | | associates with hax-1, | | | | |
| | | | | | | | | | | | | | | through binding to its | | | | |
| | | | | | | | | | | | | | | c-terminal region, may | | | | |
| | | | | | | | | | | | | | | also be involved in the | | | | |
| | | | | | | | | | | | | | | regulation of gene | | | | |
| | | | | | | | | | | | | | | expression (by | | | | |
| ET63188 | 1.00 | −2.00 | −1 95 | 0.95 | 0.01 | 1.68 | 1.62 | 0.27 | 3.46 | 0.42 | fibroblast | ET63188 | | | 2 36.0 cM | Cytokine | | |
| | | | | | | | | | | | activation | | | | | | | |
| | | | | | | | | | | | protein; Fap | | | | | | | |
| Msa 3665.0 | 1.00 | −1.63 | −0.56 | 1.08 | 0.12 | 1.68 | 2.81 | 0.31 | 3.45 | 0.40 | DNA segment, | AA116604 | | cathepsin z, cysteine | 2 24.0 cM | Proteolytic | | |
| | | | | | | | | | | | Chr 2, Wayne | | | proteinase, papain | | | | |
| | | | | | | | | | | | State University | | | superfamily member, | | | | |
| | | | | | | | | | | | 143, expressed; | | | ubiquitously | | | | |
| | | | | | | | | | | | D2Wsu143c | | | expressed, involved | | | | |
| | | | | | | | | | | | | | | normal intracellular | | | | |
| | | | | | | | | | | | | | | degradation | | | | |
| U73478 | 1.00 | −1.21 | 2.43 | 0.37 | 2.32 | 0.99 | 3.67 | 0.65 | 3.42 | 0.73 | acidic nuclear | U73478 | | | 9 36.0 cM | | | |
| | | | | | | | | | | | phosphoprotein | | | | | | | |
| | | | | | | | | | | | 32; Anp32 | | | | | | | |
| Msa.2385.0 | 1.00 | −1.21 | −1.71 | 1.27 | 0.58 | 2.13 | 2.56 | 0.37 | 3.41 | 0.25 | insulin-like | X81582 | insulin-like | binding proteins may | | Extracellular | | |
| | | | | | | | | | | | growth factor | | growth factor | act to distribute the | | Protein | | |
| | | | | | | | | | | | binding protein | | binding protein | igfs among the body | | | | |
| | | | | | | | | | | | 4; Igfbp4 | | 4 precursor | fluid compartments, to | | | | |
| | | | | | | | | | | | | | (igfbp-4) (ibp-4) | protect the body from | | | | |
| | | | | | | | | | | | | | (igf binding | possible hypoglycemic | | | | |
| | | | | | | | | | | | | | protein 4). | effects of the 1gfs | | | | |
| AF003691 | 1.00 | 3.40 | −1.88 | 1.54 | 5.02 | 3.92 | −1.08 | 1.43 | 3.41 | 6.12 | keratin-associated | AF003691 | | | | | | |
| | | | | | | | | | | | protein 14; | | | | | | | |
| | | | | | | | | | | | Krtap14 | | | | | | | |
| AA030688 | 1.00 | −1.40 | −0.06 | 0.82 | 0.38 | 1.48 | 5.02 | 2.01 | 3.41 | 0.38 | EST; Unknown | AA030688 | | | | EST; | | |
| | | | | | | | | | | | | | | | | Unknown | | |
| AA189512 | 1.00 | −1.76 | 0.59 | 0.90 | 0.09 | 1.32 | 2.95 | 0.73 | 3.40 | 0.81 | lymphocyte | AA189512 | | | | | | |
| | | | | | | | | | | | antigen 86; Ly86 | | | | | | | |
| L07063 | 1.00 | −2.99 | −1.77 | 1.08 | −0.05 | 1.46 | 2.71 | 0.25 | 3.39 | 0.45 | FK506 binding | L07063 | 65 kda fk506- | aka fkbp65 -- fkbp65 | 11 58.0 | Intracellular | | |
| | | | | | | | | | | | protein 6 (65 | | binding protein | is a member of the | cM | Protein | | |
| | | | | | | | | | | | kDa); Fkbp6 | | precursor (ec | fk56-binding protein | | | | |
| | | | | | | | | | | | | | 5.2.1.8) (1kbp65) | class of | | | | |
| | | | | | | | | | | | | | (fkbprp) | immunophilins and is | | | | |
| | | | | | | | | | | | | | (peptidyl-prolyl | the only member | | | | |
| | | | | | | | | | | | | | cis-trans | reported to contain | | | | |
| | | | | | | | | | | | | | isomerase) | four peptidylprolyl cis- | | | | |
| | | | | | | | | | | | | | (pplase) | trans isomerase | | | | |
| | | | | | | | | | | | | | (rotamase) | domains and an | | | | |
| | | | | | | | | | | | | | (immunophilin | unrelated | | | | |
| | | | | | | | | | | | | | fkbp65). | | | | | |
| C79010 | 1.00 | −1.16 | 0.57 | 0.74 | 0.20 | 1.36 | 2.95 | 0.16 | 3.38 | 0.63 | Src-associated | C79010 | | | | ?? | | |
| | | | | | | | | | | | adaptor protein; | | | | | | | |
| | | | | | | | | | | | Saps | | | | | | | |
| M16355 | 1.00 | −8.28 | 0.30 | 1.12 | 0.93 | 2.07 | 2.65 | 0.61 | 3.38 | 1.03 | major urinary | M16355 | major urinary | the mup proteins are | 4 27.8 cM | Extracellular | | |
| | | | | | | | | | | | protein 1; Mup1 | | protein 1 | lipocalins, and | | Protein | | |
| | | | | | | | | | | | | | precursor | apparently take part in | | | | |
| | | | | | | | | | | | | | (mup 1). | the transport of | | | | |
| | | | | | | | | | | | | | | pheromones. though | | | | |
| | | | | | | | | | | | | | | secreted in the urine | | | | |
| | | | | | | | | | | | | | | they are produced in | | | | |
| | | | | | | | | | | | | | | the liver or in the | | | | |
| | | | | | | | | | | | | | | lachrymal mammary, | | | | |
| | | | | | | | | | | | | | | and submaxillary | | | | |
| | | | | | | | | | | | | | | glands | | | | |
| C76049 | 1.00 | 1.55 | 1.84 | 0.21 | 1.84 | 0.32 | 5.59 | 0.86 | 3.38 | 0.87 | EST; Unknown | C76049 | | | EST; | | | |
| | | | | | | | | | | | | | | | | Unknown | | |
| X80478 | 1.00 | −1.17 | −1.47 | 0.70 | 0.44 | 2.11 | 2.02 | 0.35 | 3.38 | 0.40 | AE-binding | X80478 | | encodes a 845-aa | | Transcription | | |
| | | | | | | | | | | | protein 1; Acbp1 | | | protein that is almost | | Factor | | |
| | | | | | | | | | | | | | | identical to mouse | | | | |
| | | | | | | | | | | | | | | adipocyte transcription | | | | |
| | | | | | | | | | | | | | | factor aebp1. it is also | | | | |
| | | | | | | | | | | | | | | expressed in a murine | | | | |
| | | | | | | | | | | | | | | osteoblastic line, but is | | | | |
| | | | | | | | | | | | | | | shut off in the final | | | | |
| | | | | | | | | | | | | | | calcification phase, | | | | |
| | | | | | | | | | | | | | | suggesting a | | | | |
| | | | | | | | | | | | | | | transcriptional | | | | |
| | | | | | | | | | | | | | | repressive eff | | | | |
| M87276 | 1.00 | 1.13 | 2.06 | 0.24 | 2.43 | 0.87 | 2.49 | 0.07 | 3.34 | 0.37 | thrombospondin | M87276 | thrombospondin 1 | thrombospondin-1 | 2 65.0 cM | Extracellular | | |
| | | | | | | | | | | | 1; Thbs1 | | precursor. | functions as a cell | | Protein | | |
| | | | | | | | | | | | | | | adhesion molecule and | | | | |
| | | | | | | | | | | | | | | also modulates cell | | | | |
| | | | | | | | | | | | | | | movement, cell | | | | |
| | | | | | | | | | | | | | | proliferation, neurite | | | | |
| | | | | | | | | | | | | | | outgrowth and | | | | |
| | | | | | | | | | | | | | | angiogenesis | | | | |
| D87967 | 1.00 | 1.02 | 0.59 | 0.61 | 0.48 | 1.55 | 3.41 | 0.31 | 3.34 | 0.33 | protein tyrosine | D87967 | | | 2 73.1 cM | Intracellular | | |
| | | | | | | | | | | | phosphatase, non | | | | | Protein | | |
| | | | | | | | | | | | receptor type | | | | | | | |
| | | | | | | | | | | | substrate 1: | | | | | | | |
| | | | | | | | | | | | Ptpns1 | | | | | | | |
| AA617405 | 1.00 | −1.26 | 1.56 | 0.56 | 1.50 | 0.41 | 4.69 | 0.77 | 3.32 | 0.66 | EST; Unknown | AA617405 | | | | EST; | | |
| | | | | | | | | | | | | | | | | Unknown | | |
| AA230776 | 1.00 | −1.20 | 0.86 | 0.52 | 1.28 | 2.33 | 2.78 | 0.57 | 3.29 | 0.37 | thymosin beta 10 | AA230776 | | beta-thymosins are a | | Structural | | |
| | | | | | | | | | | | (prothymosin beta | | | family of monomeric | | Protein | | |
| | | | | | | | | | | | 10) | | | actin sequestering | | | | |
| | | | | | | | | | | | | | | peptides that regulate | | | | |
| | | | | | | | | | | | | | | actin dynamics within | | | | |
| | | | | | | | | | | | | | | the cells. during | | | | |
| | | | | | | | | | | | | | | embyrogenesis the | | | | |
| | | | | | | | | | | | | | | control of actin | | | | |
| | | | | | | | | | | | | | | polymerization is | | | | |
| | | | | | | | | | | | | | | essential in processes | | | | |
| | | | | | | | | | | | | | | such as cell migration, | | | | |
| Msa.2614.0 | 1.00 | −1.39 | 0.50 | 0.64 | 1.59 | 0.53 | 3.76 | 0.38 | 3.28 | 0.68 | properdin factor, | X12905 | properdin | a positive regulator of | X 6.2 cM | Extracellular | | |
| | | | | | | | | | | | complement; Pfc | | (fragment). | the alternate pathway | | Protein | | |
| | | | | | | | | | | | | | | of complement. it | | | | |
| | | | | | | | | | | | | | | binds to and stabilizes | | | | |
| | | | | | | | | | | | | | | the c3-and c5- | | | | |
| | | | | | | | | | | | | | | convertase enzyme | | | | |
| | | | | | | | | | | | | | | complexes | | | | |
| AF017989 | 1.00 | −2.81 | −2.56 | 1.30 | 0.09 | 2.39 | 1.91 | 0.42 | 3.28 | 0.60 | stromal cell | AF017989 | | a soluble frizzled | 3 38.5 | Extracellular | | |
| | | | | | | | | | | | derived factor 5; | | | related protein that | cM | Protein | | |
| | | | | | | | | | | | Sdf5 | | | may act as a wnt | | | | |
| | | | | | | | | | | | | | | antagonist. | | | | |
| M24509 | 1.00 | 1.04 | 2.36 | 0.66 | 2.25 | 0.67 | 3.25 | 0.83 | 3.27 | 0.73 | ferritin heavy | M24509 | ferritin heavy | ferritin is an | 19 2.0 cM | | | |
| | | | | | | | | | | | chain; Fth | | chain (ferritin h | intracellular molecule | | | | |
| | | | | | | | | | | | | | subunit). | that stores iron in a | | | | |
| | | | | | | | | | | | | | | soluble, nontoxic, | | | | |
| | | | | | | | | | | | | | | readily available form. | | | | |
| | | | | | | | | | | | | | | the functional | | | | |
| | | | | | | | | | | | | | | molecule, which is | | | | |
| | | | | | | | | | | | | | | composed of 24 | | | | |
| | | | | | | | | | | | | | | chains, is roughly | | | | |
| | | | | | | | | | | | | | | spherical and contains | | | | |
| | | | | | | | | | | | | | | a central cavity in | | | | |
| | | | | | | | | | | | | | | which the polymeric | | | | |
| | | | | | | | | | | | | | | ferric iron core is | | | | |
| | | | | | | | | | | | | | | deposited | | | | |
| Msa.4744.0 | 1.00 | 1.13 | 2.87 | 0.83 | 2.31 | 1.28 | 3.00 | 2.22 | 3.27 | 1.91 | topoisomerase | W10047 | dna | the reaction catalyzed | 2 92.0 cM | | | |
| | | | | | | | | | | | (DNA) 1; Top1 | | topoisomerase i | by topoisomerases | | | | |
| | | | | | | | | | | | | | (ec 5.99.1.2). | leads to the conversion | | | | |
| | | | | | | | | | | | | | | of one topological | | | | |
| | | | | | | | | | | | | | | isomer of dna to | | | | |
| | | | | | | | | | | | | | | another. | | | | |
| U29539 | 1.00 | −1.52 | 0.42 | 0.73 | 1.06 | 2.21 | 3.37 | 0.68 | 3.25 | 0.38 | lysosomal- | U29539 | lysosomal- | the expression pattern | | Proteolytic | | |
| | | | | | | | | | | | associated protein | | associated | of the gene together | | | | |
| | | | | | | | | | | | transmembrane 5; | | multitrans- | with preliminary | mu | | | |
| | | | | | | | | | | | Laptm5 | | membrane | evidence that the | | | | |
| | | | | | | | | | | | | | protein (retinoic | protein interacts with | | | | |
| | | | | | | | | | | | | | acid-inducible e3 | ubiquitin indicates that | | | | |
| | | | | | | | | | | | | | protein). | the protein may have a | | | | |
| | | | | | | | | | | | | | | special functional role | | | | |
| | | | | | | | | | | | | | | during embyrogenesis | | | | |
| | | | | | | | | | | | | | | and in adult | | | | |
| | | | | | | | | | | | | | | hematopoietic cells. it | | | | |
| | | | | | | | | | | | | | | maybe | | | | |
| AA125580 | 1.00 | −1.07 | 1.43 | 0.44 | 1.46 | 0.06 | 3.65 | 0.56 | 3.22 | 0.64 | synaptosomal- | AA125580 | | | 2 61.8 cM | | | |
| | | | | | | | | | | | associated | | | | | | | |
| | | | | | | | | | | | protein, | | | | | | | |
| | | | | | | | | | | | 23kD; Snap23 | | | | | | | |
| X93328 | 1.00 | −1.75 | 0.32 | 0.79 | 0.38 | 1.69 | 3.03 | 0.17 | 3.21 | 0.44 | EGF-like module | X93328 | cell surface | probably involved in | 17 34.3 | Cell Surface | | |
| | | | | | | | | | | | containing, | | glycoprotein emr1 | cell adhesion within | cM | Protein | | |
| | | | | | | | | | | | mucin-like, | | precursor (emr1 | tissues and receptor | | | | |
| | | | | | | | | | | | hormone | | hormone | signalling. | | | | |
| | | | | | | | | | | | receptor-like | | receptor) | | | | | |
| | | | | | | | | | | | sequence 1; Emr1 | | (cell surface | | | | | |
| | | | | | | | | | | | | | glycoprotein | | | | | |
| | | | | | | | | | | | | | f4/80). | | | | | |
| Msa.35983.0 | 1.00 | −1.10 | −1.46 | 1.04 | 0.12 | 1.54 | 2.65 | 0.50 | 3.19 | 0.26 | secreted | AA123395 | osteopontin | binds tightly to | 5 56.0 cM | Cytokine | | |
| | | | | | | | | | | | phosphoprotein 1; | | precursor (bone | hydroxyapatite. | | | | |
| | | | | | | | | | | | Spp1 | | sialoprotein 1) | appears to form an an | | | | |
| | | | | | | | | | | | | | (minopontin) | integral part of the | (mi | | | |
| | | | | | | | | | | | | | (early i | mineralized matrix. | | | | |
| | | | | | | | | | | | | | lymphocyte | probably important to | | | | |
| | | | | | | | | | | | | | activation 1 pro- | cell-matrix interaction. | | | | |
| | | | | | | | | | | | | | tein) (secreted | | | | | |
| | | | | | | | | | | | | | phosphoprotein 1) | | | | | |
| | | | | | | | | | | | | | (spp-1)(2ar) | | | | | |
| | | | | | | | | | | | | | (calcium oxalate | | | | | |
| | | | | | | | | | | | | | crystal growth | | | | | |
| | | | | | | | | | | | | | | inhibitor protein) | | | | |
| Msa.2173.0 | 1.00 | 1.16 | 0.76 | 0.57 | 0.37 | 1.73 | 3.13 | 0.44 | 3.17 | 0.38 | inhibin beta-A; | X69619 | inhibin beta a | inhibin is a gonadal | 13 10.0 | Cytokine | | |
| | | | | | | | | | | | Inhba | | chain precursor | glycopeptide that | cM | | | |
| | | | | | | | | | | | | | (activin | inhibits the secretion | | | | |
| | | | | | | | | | | | | | beta-a chain). | of follitropin by the | | | | |
| | | | | | | | | | | | | | | pituitary gland. on the | | | | |
| | | | | | | | | | | | | | | other hand activin | | | | |
| | | | | | | | | | | | | | | activates the secretion | | | | |
| | | | | | | | | | | | | | | of follitropin. activin | | | | |
| | | | | | | | | | | | | | | is also important in | | | | |
| | | | | | | | | | | | | | | embyronic axial | | | | |
| | | | | | | | | | | | | | | development | | | | |
| AF004874 | 1.00 | −1.80 | −0.96 | 0.71 | 0.93 | 2.54 | 3.03 | 1.07 | 3.16 | 0.29 | latent | AF004874 | | human protein is | 12 D | Unknown | | |
| | | | | | | | | | | | transforming | | | structurally similar to | | | | |
| | | | | | | | | | | | growth factor | | | fibrillin, plays a role | | | | |
| | | | | | | | | | | | beta binding | | | in bone biology? 1tbp- | | | | |
| | | | | | | | | | | | protein 2; Ltbp2 | | | 2 gene expression in | | | | |
| | | | | | | | | | | | | | | mouse embryos was | | | | |
| | | | | | | | | | | | | | | restricted to cartilage | | | | |
| | | | | | | | | | | | | | | perichondrium and | | | | |
| | | | | | | | | | | | | | | blood vessels, a | | | | |
| | | | | | | | | | | | | | | somewhat surprising | | | | |
| | | | | | | | | | | | | | | result since other 1tbp | | | | |
| L38971 | 1.00 | −2.23 | −1.09 | 0.80 | 1.13 | 2.99 | 0.72 | 1.31 | 3.15 | 0.99 | integral | L38971 | integral | | X 37.0 cM | | | |
| | | | | | | | | | | | membrane | | membrane | | | | | |
| | | | | | | | | | | | protein 2; Itm2 | | protein 2a (e25 | | | | | |
| | | | | | | | | | | | | | protein). | | | | | |
| Msa.64.0 | 1.00 | −1.16 | 0.10 | 0.84 | 0.73 | 1.75 | 3.06 | 0.40 | 3.15 | 0.40 | collagen binding | D12907 | 47 kda heat shock | binds specifically to | | Intracellular | | |
| | | | | | | | | | | | protein 1; Cbp1 | | protein precursor | collagen. could be | | Protein | | |
| | | | | | | | | | | | | | (collagen-binding | involved as a | | | | |
| | | | | | | | | | | | | | protein 1) (serine | chapcrone in the | | | | |
| | | | | | | | | | | | | | protease inhibitor | biosynthetic pathway | | | | |
| | | | | | | | | | | | | | i6) | of collagen | | | | |
| M31418 | 1.00 | −1.10 | 1.14 | 0.90 | 1.73 | 2.88 | 3.24 | 0.81 | 3.13 | 0.18 | interferon | M31418 | interferon- | inhibits the | 1,1 95 2 | | | |
| | | | | | | | | | | | activated gene | | activatable | transcriptional activity | cM | | | |
| | | | | | | | | | | | 202A,interferon | | protein 202a | of several | | | | |
| | | | | | | | | | | | activated gene | | (ifi-202a) | transcription factors, | | | | |
| | | | | | | | | | | | 202B; Ifi202a, | | (interferon- | including nf-kappa-b | | | | |
| | | | | | | | | | | | Ifi202b | | inducible protein | p5 and p65, ap-1, c- | | | | |
| | | | | | | | | | | | | | p202a)., | fos, c-jun, e2f-1, e2f-4, | | | | |
| | | | | | | | | | | | | | interferon- | myod and myogenin. | | | | |
| | | | | | | | | | | | | | activatable | inhibits the | | | | |
| | | | | | | | | | | | | | protein 202b | transcriptional activity | | | | |
| | | | | | | | | | | | | | (ifi-202b) | of p53.,inhibits the | | | | |
| | | | | | | | | | | | | | (interferon- | transcriptional activity | | | | |
| | | | | | | | | | | | | | inducible protein | | | | | |
| | | | | | | | | | | | | | p202b). | | | | | |
| AA177300 | 1.00 | −1 63 | −1.28 | 0.98 | 0.84 | 2.56 | 3.22 | 1.35 | 3.11 | 0.45 | DNA segment, | AA177300 | | seven transmembrane | 13 6.0 cM | Cell Surface | | |
| | | | | | | | | | | | Chr 13, Abbott 1 | | | domain protein, | | Protein | | |
| | | | | | | | | | | | expressed; | | | upregulated during | | | | |
| | | | | | | | | | | | D13Abb1e | | | kidney development. | | | | |
| X56304 | 1.00 | −1.02 | −2.37 | 1.63 | 0.48 | 2.28 | 2.63 | 0.39 | 3.10 | 0.34 | tenascin C; Tnc | X56304 | | extracellular matrix | 4 32.2 cM | ECM | | |
| | | | | | | | | | | | | | | glycoprotein expressed | | (Matrix | | |
| | | | | | | | | | | | | | | in developing brain, | | Prot) | | |
| | | | | | | | | | | | | | | mesenchyme, and | | | | |
| | | | | | | | | | | | | | | cartilage, osteoblasts, | | | | |
| | | | | | | | | | | | | | | periosteal and | | | | |
| | | | | | | | | | | | | | | perichondrial cells, | | | | |
| | | | | | | | | | | | | | | and articular surfaces, | | | | |
| | | | | | | | | | | | | | | and its maintained into | | | | |
| | | | | | | | | | | | | | | adult stages in some | | | | |
| | | | | | | | | | | | | | | tissues notably the | | | | |
| U05265 | 1.00 | −1.14 | 0.38 | 0.61 | 0.20 | 1.56 | 4.01 | 0.57 | 3.06 | 0.66 | glycoprotein 49 | U05265 | mast cell surface | preferentially | 10 32.0 | Cell Surface | | |
| | | | | | | | | | | | B; Gp49b | | glycoprotein | expressed on mouse | cM | Protein | | |
| | | | | | | | | | | | | | gp49a precursor., | interleukin-3- | | | | |
| | | | | | | | | | | | | | mast cell surface | dependent, bone | | | | |
| | | | | | | | | | | | | | glycoprotein | marrow-derived mast | | | | |
| | | | | | | | | | | | | | gp49b precursor. | cells, which are | | | | |
| | | | | | | | | | | | | | | immature progenitor | | | | |
| | | | | | | | | | | | | | | cells. members of the | | | | |
| | | | | | | | | | | | | | | protein family are | | | | |
| | | | | | | | | | | | | | | alternative splices of | | | | |
| | | | | | | | | | | | | | | gene Gp49 | | | | |
| D12907 | 1.00 | −1.56 | 0.04 | 0.74 | 0.80 | 1.99 | 2.84 | 0.37 | 3.03 | 0.15 | collagen binding | D12907 | 47 kda heat shock | binds specifically to | | Intracellular | | |
| | | | | | | | | | | | protein 1; Cbp1 | | protein precursor | collagen. could be | | Protein | | |
| | | | | | | | | | | | | | (collagen-binding | involved as a | | | | |
| | | | | | | | | | | | | | protein 1) (serine | chaperone in the | | | | |
| | | | | | | | | | | | | | protease inhibitor | biosynthetic pathway | | | | |
| | | | | | | | | | | | | | i6) | of collagen | | | | |
| D86422 | 1.00 | 7.64 | 1.04 | 1.06 | 7.03 | 8.15 | 0.24 | 1.30 | 3.03 | 1.86 | keratin-associated | D86422 | | | | | | |
| | | | | | | | | | | | protein 8-2; | | | | | | | |
| | | | | | | | | | | | Krtsp8-2 | | | | | | | |
| V00802 | 1.00 | 3.13 | 1.02 | 0.89 | 2.20 | 0.79 | 0.74 | 0.96 | 3.02 | 0.42 | V00802 | | | | EST; | | | |
| | | | | | | | | | | | | | | | | Unknown | | |
| AA408463 | 1.00 | 1.15 | −0.68 | 0.55 | 0.66 | 2.06 | 2.37 | 0.79 | 3.02 | 0.25 | probable match to | AA408463 | | vasculo-endothelial | | Cell Surface | | |
| | | | | | | | | | | | cadherin 5 | | | (ve)-cadherin is | | Protein | | |
| | | | | | | | | | | | | | | specifically expressed | | | | |
| | | | | | | | | | | | | | | in endothelial cells. | | | | |
| | | | | | | | | | | | | | | expressed in | | | | |
| | | | | | | | | | | | | | | ubiquitously in | | | | |
| | | | | | | | | | | | | | | vascular structures. | | | | |
| | | | | | | | | | | | | | | cadherins act as cell | | | | |
| | | | | | | | | | | | | | | adhesion receptors | | | | |
| U52524 | 1.00 | −1.35 | 0.32 | 0.62 | 0.41 | 1.81 | 3.16 | 0.79 | 3.00 | 0.56 | hyaluronan | U52524 | hyaluronan | play a role in | 15 31.2 | Other | | |
| | | | | | | | | | | | synthase 2;Has2 | | synthase | hyaluronan/hyaluronic | cM | | | |
| | | | | | | | | | | | | | 2(ec 2.4.1.—) | acid(ha) synthesis. | | | | |
| | | | | | | | | | | | | | (hyaluronate | | | | | |
| | | | | | | | | | | | | | synthase 2) | | | | | |
| | | | | | | | | | | | | | (hyaluronic acid | | | | | |
| | | | | | | | | | | | | | synthase 2) (ha | | | | | |
| | | | | | | | | | | | | | synthase 2) | | | | | |
| X14951 | 1.00 | −1.02 | 0.33 | 0.69 | 0.52 | 2.07 | 2.90 | 0.25 | 2.99 | 0.46 | integrin beta 2 | X14951 | cell surface | associates with alpha-1 | 10 41.5 | Cell Surface | | |
| | | | | | | | | | | | (Cd18); Itgb2 | | adhesion | (1fa-1) to interact with | cM | Protein | | |
| | | | | | | | | | | | | | glycoproteins 1fa- | icam-1, and with alpha | | | | |
| | | | | | | | | | | | | | 1/cr3/p150,95 | m(mac-1) or alpha-x | | | | |
| | | | | | | | | | | | | | beta-subunit | to form the receptor | | | | |
| | | | | | | | | | | | | | precursor | for the ic-3b fragment | | | | |
| | | | | | | | | | | | | | (integrin beta-2) | of the third | | | | |
| | | | | | | | | | | | | | (cd18 antigen) | complement | | | | |
| | | | | | | | | | | | | | (complement | component | | | | |
| | | | | | | | | | | | | | receptor c3 beta | | | | | |
| AA537404 | 1.00 | −1.18 | −0.66 | 0.85 | 0.43 | 1.49 | 2.74 | 0.44 | 2.97 | 0.34 | thymosin beta-10; | AA537404 | | expressed at relatively | | Structural | | |
| | | | | | | | | | | | from rat | | | high levels in | | Protein | | |
| | | | | | | | | | | | | | | embryonic tissues, and | | | | |
| | | | | | | | | | | | | | | its mrna is abundant in | | | | |
| | | | | | | | | | | | | | | a variety of tumors | | | | |
| | | | | | | | | | | | | | | and tumor cell lines. a | | | | |
| | | | | | | | | | | | | | | major intracellular g- | | | | |
| | | | | | | | | | | | | | | actin binding protein. | | | | |
| | | | | | | | | | | | | | | (a) plays a significant | | | | |
| | | | | | | | | | | | | | | and possibly | | | | |
| | | | | | | | | | | | | | | obligatory role in | | | | |
| | | | | | | | | | | | | | | cellular | | | | |
| AA285635 | 1.00 | 2.08 | −0.32 | 0.77 | 3.37 | 2.34 | 3.01 | 1.30 | 2.95 | 0.72 | ectoplacental | AA285635 | | | | EST; | | |
| | | | | | | | | | | | cone, invasive | | | | | Unknown | | |
| | | | | | | | | | | | trophoblast | | | | | | | |
| | | | | | | | | | | | giant cells, | | | | | | | |
| | | | | | | | | | | | extraembryonic | | | | | | | |
| | | | | | | | | | | | cctoderm and | | | | | | | |
| | | | | | | | | | | | chorion | | | | | | | |
| | | | | | | | | | | | sequence 21; | | | | | | | |
| | | | | | | | | | | | Epcs21- | | | | | | | |
| | | | | | | | | | | | pending | | | | | | | |
| Msa.8157.0 | 1.00 | −1.28 | −0.93 | 0.86 | 0.17 | 1.94 | 2.89 | 0.07 | 2.95 | 0.33 | cathepsin S Ctss | AA089333 | | the cathepsins are | 3 42.7 cM | Proteolytic | | |
| | | | | | | | | | | | | | | mammalian lysosomal | | | | |
| | | | | | | | | | | | | | | cysteine proteinases. | | | | |
| | | | | | | | | | | | | | | they play an important | | | | |
| | | | | | | | | | | | | | | role in protein | | | | |
| | | | | | | | | | | | | | | catabolism within the | | | | |
| | | | | | | | | | | | | | | cell, and may be | | | | |
| | | | | | | | | | | | | | | involved in tumor | | | | |
| | | | | | | | | | | | | | | metastasis | | | | |
| D50586 | 1.00 | 1.00 | 0.03 | 0.57 | 0.22 | 1.43 | 3.12 | 0.85 | 2.95 | 0.33 | tissue factor | D50586 | | | 6 1.0 cM | | | |
| | | | | | | | | | | | pathway | | | | | | | |
| | | | | | | | | | | | inhibitor 2 Tfpi2 | | | | | | | |
| M74149 | 1.00 | −1.31 | −1.11 | 1.25 | 0.64 | 1.77 | 2.40 | 0.75 | 2.94 | 0.38 | creatine kinase | M74149 | creatine kinase b | reversibly catalyzes | 12 55.0 | Metabolic | | |
| | | | | | | | | | | | brain, Ckb | | chain (ec 2.7 3.2) | the transfer of | cM | | | |
| | | | | | | | | | | | | | (b-ck). | phosphate between atp | | | | |
| | | | | | | | | | | | | | | and various | | | | |
| | | | | | | | | | | | | | | phosphogens (e.g. | | | | |
| | | | | | | | | | | | | | | creatine kinase | | | | |
| | | | | | | | | | | | | | | isoenzymes play a | | | | |
| | | | | | | | | | | | | | | central role in energy | | | | |
| | | | | | | | | | | | | | | transduction in tissues | | | | |
| | | | | | | | | | | | | | | with large, fluctuating | | | | |
| | | | | | | | | | | | | | | energy demands, such | | | | |
| | | | | | | | | | | | | | | as skeletal muscle, | | | | |
| | | | | | | | | | | | | | | heart, brain, and | | | | |
| Msa.805.0 | 1.00 | 1.60 | 2.03 | 1.10 | 2.06 | 0.82 | 1.55 | 0.25 | 2.94 | 1.18 | immunoglobulin | J00475 | | | 12 58.0 | Hemostasis | | |
| | | | | | | | | | | | heavy chain 1 | | | | cM | | | |
| | | | | | | | | | | | (serum IgG2a), | | | | | | | |
| | | | | | | | | | | | immunoglobu | | | | | | | |
| | | | | | | | | | | | lin heavy chain 3 | | | | | | | |
| | | | | | | | | | | | (serum | | | | | | | |
| | | | | | | | | | | | IgG2b), | | | | | | | |
| | | | | | | | | | | | immunoglobulin | | | | | | | |
| | | | | | | | | | | | heavy chain 4 | | | | | | | |
| | | | | | | | | | | | (serum | | | | | | | |
| | | | | | | | | | | | IgG1), | | | | | | | |
| | | | | | | | | | | | immunoglobulin | | | | | | | |
| | | | | | | | | | | | heavy chain 6 | | | | | | | |
| | | | | | | | | | | | (heavy chain of | | | | | | | |
| | | | | | | | | | | | 1gM); Igh-1, | | | | | | | |
| | | | | | | | | | | | Igh-3, Igh-4, | | | | | | | |
| | | | | | | | | | | | Igh-6 | | | | | | | |
| Msa.7614.0 | 1.00 | −1.36 | −0.23 | 0.68 | 0.48 | 1.57 | 4.41 | 0.21 | 2.93 | 0.49 | homolog(84%) of | W18778 | | | | EST; | | |
| | | | | | | | | | | | human beta- | | | | | Unknown | | |
| | | | | | | | | | | | tubulin | | | | | | | |
| Msa 3176.0 | 1.00 | 1.60 | 0.10 | 0.64 | 1.34 | 0.17 | 1.78 | 0.17 | 2.91 | 0.37 | cathepsin E; Ctse | X97399 | cathepsin e | due of its intracellular | 1 69.1 cM | Proteolytic | | |
| | | | | | | | | | | | | | precursor (ec | location and | | | | |
| | | | | | | | | | | | | | 3.4.23.34). | distribution in | | | | |
| | | | | | | | | | | | | | | lymphoid associated | | | | |
| | | | | | | | | | | | | | | tissue, it may have a | | | | |
| | | | | | | | | | | | | | | role in immune | | | | |
| | | | | | | | | | | | | | | function | | | | |
| X60367-2 | 1.00 | 1.25 | −0.64 | 0.89 | 1.48 | 0.40 | 1.75 | 0.34 | 2.91 | 0.22 | retinol binding | X60367 | retinol-binding | the rbp1 gene encodes | 9 52.0 cM | Regulatory | | |
| | | | | | | | | | | | protein 1, | | protein i, cellular | crbp, a protein present | | | | |
| | | | | | | | | | | | cellular; Rbp1 | | (mcrbpi). | in a wide variety of | | | | |
| | | | | | | | | | | | | | | adult rat tissues but | | | | |
| | | | | | | | | | | | | | | most abundant | | | | |
| | | | | | | | | | | | | | | in liver | | | | |
| | | | | | | | | | | | | | | and kidney | | | | |
| AA542220 | 1.00 | 1.33 | 0.43 | 1.01 | 2.01 | 3.11 | 3.69 | 1.55 | 2.90 | 0.77 | TBX1 protein; | AA542220 | thx1 protein | estrogen treatment | | Intracellular | | |
| | | | | | | | | | | | TBX1 | | (t-box protein 1) | resulted in a rapid and | | Protein | | |
| | | | | | | | | | | | | | (testis-specific | transient increase in | | | | |
| | | | | | | | | | | | | | t-box protein) | eet-1 messenger ma; | | | | |
| | | | | | | | | | | | | | (fragment). | steady state levels | | | | |
| | | | | | | | | | | | | | | peaked between 2-3 h, | | | | |
| | | | | | | | | | | | | | | returning to basal | | | | |
| | | | | | | | | | | | | | | levels by 6 h. this | | | | |
| | | | | | | | | | | | | | | increase was not | | | | |
| | | | | | | | | | | | | | | abolished by | | | | |
| | | | | | | | | | | | | | | pretreatment with | | | | |
| | | | | | | | | | | | | | | cycloheximide, | | | | |
| | | | | | | | | | | | | | | indicating | | | | |
| ab000822 | 1.00 | 1.09 | 1.15 | 0.61 | 2.65 | 1.54 | 3.02 | 0.68 | 2.90 | 0.52 | synaptosomal- | AB000822 | | | 2 61.8 cM | | | |
| | | | | | | | | | | | associated | | | | | | | |
| | | | | | | | | | | | protein, | | | | | | | |
| | | | | | | | | | | | 23kD: Snap23 | | | | | | | |
| ab009287 | 1.00 | −1.10 | −0.70 | 0.66 | 0.26 | 1.71 | 2.69 | 0.03 | 2.89 | 0.42 | CD68 antigen; | AB009287 | macrosialin | a.k.a. macrosialin --the | 11 39.0 | Cell Surface | | |
| | | | | | | | | | | | Cd68 | | precursor (cd68 | glycoprotein | cM | Protein | | |
| | | | | | | | | | | | | | antigen). | macrosialin is | | | | |
| | | | | | | | | | | | | | | expressed specifically | | | | |
| | | | | | | | | | | | | | | in murine monocytes | | | | |
| | | | | | | | | | | | | | | and macrophages | | | | |
| M74227 | 1.00 | −1.62 | −1.17 | 0.88 | 0.50 | 1.83 | 1.58 | 0.28 | 2.88 | 0.15 | peptidylprolyl | M74227 | peptidyl-prolyl | ppiases accelerate the | | Intracellular | | |
| | | | | | | | | | | | isomerase C; Ppic | | cis-trans | folding of proteins. | | Protein | | |
| | | | | | | | | | | | | | isomerase c | | | | | |
| | | | | | | | | | | | | | (ec 5.2.1.8) | | | | | |
| | | | | | | | | | | | | | (ppiase) | | | | | |
| | | | | | | | | | | | | | (rotamase) | | | | | |
| | | | | | | | | | | | | | (cyclophilin c). | | | | | |
| Msa.1629.0 | 1.00 | 1.00 | 0.48 | 1.03 | 0.32 | 1.52 | 2.16 | 0.22 | 2.88 | 0.26 | proteosome | U22031 | proteasome | the proteasome is a | 17 18.61 | Proteolytic | | |
| | | | | | | | | | | | (prosome, | | component e13 | multicatalytic | cM | | | |
| | | | | | | | | | | | macropain) | | precursor (ec | proteinase complex | | | | |
| | | | | | | | | | | | subunit, beta | | 3.4.99.46) | which is characterized | | | | |
| | | | | | | | | | | | type 8 (large | | (macropain | by its ability to cleave | | | | |
| | | | | | | | | | | | multifunctional | | subunit c13) | peptides with arg, phe, | | | | |
| | | | | | | | | | | | protease 7); | | (multicatalytic | tyr, leu, and glu | | | | |
| | | | | | | | | | | | Psmb8 | | endopeptidase | adjacent to the leaving | | | | |
| | | | | | | | | | | | | | complex subunit | group at neutral or | | | | |
| | | | | | | | | | | | | | c13). | slightly basic ph. the | | | | |
| | | | | | | | | | | | | | | protteasome has an atp- | | | | |
| | | | | | | | | | | | | | | dependent proteolytic | | | | |
| | | | | | | | | | | | | | | activity. this subunit | | | | |
| | | | | | | | | | | | | | | may be involved in | | | | |
| X04648 | 1.00 | 1.05 | −0.62 | 0.83 | 0.26 | 2.21 | 3.17 | 0.23 | 2.87 | 0.37 | Fc receptor, IgG, | X04648 | low affinity | a second low affinity | 1 92.3 cM | Receptor | | |
| | | | | | | | | | | | low affinity IIb; | | immunoglobulin | receptor for the fc | | | | |
| | | | | | | | | | | | Fcgr2b | | gamma fc region | portion of igg. | | | | |
| | | | | | | | | | | | | | receptor ii | fc\gammarii and | | | | |
| | | | | | | | | | | | | | precursor | fc\gammarii receptors | | | | |
| | | | | | | | | | | | | | (fc-gamma rii) | are identical to low | | | | |
| | | | | | | | | | | | | | (fcrii) (igg fc | affinity receptor for | | | | |
| | | | | | | | | | | | | | receptor ii beta) | igc on mouse mast | | | | |
| | | | | | | | | | | | | | (fc gamma | cells and | | | | |
| | | | | | | | | | | | | | receptor iib) | macrophages. | | | | |
| | | | | | | | | | | | | | (fcgammariib). | fc\gammarii is | | | | |
| | | | | | | | | | | | | | | immunologically | | | | |
| | | | | | | | | | | | | | | indicated | | | | |
| M64086 | 1.00 | −1.36 | −3.46 | 4.25 | 1.63 | 3.55 | 4.80 | 1.51 | 2.87 | 0.36 | serine protease | M64086 | | | | Proteolytic | | |
| | | | | | | | | | | | inhibitor 2-2; | | | | | | | |
| | | | | | | | | | | | Spi2-2 | | | | | | | |
| Msa.27449.0 | 1.00 | 1.12 | −1.53 | 0.84 | 0.19 | 1.58 | 2.12 | 0.37 | 2.87 | 0.09 | secreted | AA066782 | osteopontin | bind | | | | |
| | | | | | | | | | | | phosphoprotein 1; | | precursor (bone | hydroxyapatite. | | (Matrix | | |
| | | | | | | | | | | | Spp1 | | sialoprotein 1) | appears to form an an | | Prot) | | |
| | | | | | | | | | | | | | (minopontin) | integral part of the | | | | |
| | | | | | | | | | | | | | (early i | mineralized matrix. | | | | |
| | | | | | | | | | | | | | lymphocyte | probably important to | | | | |
| | | | | | | | | | | | | | activation 1 | cell-matrix interaction. | | | | |
| | | | | | | | | | | | | | protein) (secreted | | | | | |
| | | | | | | | | | | | | | phosphoprotein 1) | | | | | |
| | | | | | | | | | | | | | (spp-1) (2ar) | | | | | |
| | | | | | | | | | | | | | (calcium oxalate | | | | | |
| | | | | | | | | | | | | | crystal growth | | | | | |
| | | | | | | | | | | | | | inhibitor protein) | | | | | |
| AA140446 | 1.00 | −1.61 | −1.09 | 0.72 | 0.32 | 1.85 | 4.28 | 2.43 | 2.84 | 0.37 | DNA segment, | AA140446 | | seven transmembrane | 13 6 0 cM | Cell Surface | | |
| | | | | | | | | | | | Chr 13, Abbott 1 | | | domain protein | | Protein | | |
| | | | | | | | | | | | expressed; | | | upregulated during | | | | |
| | | | | | | | | | | | D13Abb1e | | | kidney development | | | | |
| AA547057 | 1.00 | 1.21 | 1.69 | 0.71 | 1.88 | 0.13 | 3.69 | 0.54 | 2.83 | 0.29 | ets variant gene 6 | AA547057 | ets-related protein | | 6 63.9 cM | Extracellular | | |
| | | | | | | | | | | | (TEL oncogene); | | tel (ets transloca- | | | Protein | | |
| | | | | | | | | | | | Etv6 | | tion variant 6). | | | | | |
| AA259726 | 1.00 | 1.02 | 0.74 | 0.62 | 2.33 | 1.10 | 3.74 | 1.63 | 2.82 | 0.44 | EST; Unknown | AA259726 | | | | EST; | | |
| | | | | | | | | | | | | | | | | Unknown | | |
| ET61206 | 1.00 | 2.05 | 1.11 | 0.76 | 1.81 | 0.45 | 0.70 | 1.08 | 2.81 | 0.31 | | ET61206 | | | | EST; | | |
| | | | | | | | | | | | | | | | | Unknown | | |
| Msa.10497.0 | 1.00 | −4.02 | 2.16 | 0.97 | 0.52 | 2.77 | 3.48 | 2.46 | 2.79 | 1.43 | | W48224 | | | | | | |
| ET62056 | 1.00 | 2.54 | −0.61 | 1.70 | 1.79 | 0.33 | 0.68 | 1.04 | 2.78 | 0.40 | immunoglobulin | ET62056 | | | | Extracellular | | |
| | | | | | | | | | | | rearranged kappa | | | | | Protein | | |
| | | | | | | | | | | | chain | | | | | | | |
| L06039 | 1.00 | −1.13 | 0.20 | 0.74 | 0.84 | 2.13 | 2.78 | 1.03 | 2.77 | 0.13 | platelet/ | L06039 | platelet | functions in cell-cell | 6 31.5 cM | Hemostasis | | |
| | | | | | | | | | | | endothelial | | endothelial | adhesion . . . expression | | | | |
| | | | | | | | | | | | cell adhesion | | cell adhesion | in lymphocytes | | | | |
| | | | | | | | | | | | molecule; Pecam | | molecule | transmigrating the | | | | |
| | | | | | | | | | | | | | precursor | endothelial cell | | | | |
| | | | | | | | | | | | | | (pecam-1) (cd31 | lining . . . the function of | | | | |
| | | | | | | | | | | | | | antigen). | pecam in the | | | | |
| | | | | | | | | | | | | | | emigration process is | | | | |
| | | | | | | | | | | | | | | not solely to bring | | | | |
| | | | | | | | | | | | | | | leukocytes into | | | | |
| | | | | | | | | | | | | | | contact with the | | | | |
| | | | | | | | | | | | | | | vascular endothelium | | | | |
| U90355 | 1 00 | −1.22 | −1.48 | 1.29 | 0.21 | 1.62 | 2.12 | 0 27 | 2.77 | 0.25 | fascin homolog 1 | U90355 | fascin. | organizes filamentous | 5 86.0 cM | Structural | | |
| | | | | | | | | | | | (actin bundling | | | actin into bundles with | | Protein | | |
| | | | | | | | | | | | protein, | | | a minimum of 4.1:1 | | | | |
| | | | | | | | | | | | Strongylocen- | | | actin/fascin ratio. | | | | |
| | | | | | | | | | | | trotus pur- | | | | | | | |
| | | | | | | | | | | | puratus): Fscn1 | | | | | | | |
| AA255186 | 1.00 | −1.71 | 0.71 | 0.70 | 0.32 | 2.47 | 3.26 | 0.66 | 2.76 | 0.24 | cathepsin S; Ctss | AA255186 | | lysosomal cysteine | 3 42.7 cM | Proteolytic | | |
| | | | | | | | | | | | | | | proteinase | | | | |
| AA030649 | 1.00 | −1.29 | −0.57 | 0.54 | 0.18 | 2.27 | 1.99 | 0.28 | 2.76 | 0.51 | procollagen, type | AA030649 | | type v collagen is a | 2 18.0 cM | ECM | | |
| | | | | | | | | | | | V, alpha 1; | | | member of group i | | (Matrix | | |
| | | | | | | | | | | | Col5a1 | | | collagen (fibrillar | | Prot) | | |
| | | | | | | | | | | | | | | forming collagen). | | | | |
| | | | | | | | | | | | | | | collagen v is | | | | |
| | | | | | | | | | | | | | | expressed in | | | | |
| | | | | | | | | | | | | | | connective tissue in | | | | |
| | | | | | | | | | | | | | | close contact with the | | | | |
| | | | | | | | | | | | | | | vascular basement | | | | |
| | | | | | | | | | | | | | | membrane in bone | | | | |
| | | | | | | | | | | | | | | skin, cartilage, tendon, | | | | |
| Msa.544.0 | 1.00 | −2.55 | −3.78 | 2.77 | −0.05 | 1.74 | 1.98 | 0.15 | 2.75 | 0.27 | procollagen, type | L02918 | | | | ECM | | |
| | | | | | | | | | | | V, alpha 2; | | | | | (Matrix | | |
| | | | | | | | | | | | Col5a2 | | | | | Prot) | | |
| AA690738 | 1.00 | −1.06 | 0.68 | 0.56 | 1.54 | 0.10 | 2.40 | 0.32 | 2.74 | 0.47 | EST; Unknown | AA690738 | | | | EST; | | |
| | | | | | | | | | | | | | | | | Unknown | | |
| AA711271 | 1.00 | −1.70 | −0.60 | 0.75 | 0.81 | 2.07 | 3.05 | 1.23 | 2.73 | 0.39 | EST; Unknown | AA711271 | | | | EST; | | |
| | | | | | | | | | | | | | | | | Unknown | | |
| L39017 | 1.00 | −1.55 | 1.61 | 0.77 | 1.47 | 2.56 | 1.52 | 1.35 | 2.72 | 0.46 | protein C | L39017 | | | 2 H1-3 | | | |
| | | | | | | | | | | | receptor, | | | | | | | |
| | | | | | | | | | | | endothelial; Procr | | | | | | | |
| Msa.38948.0 | 1.00 | −1.30 | −1.35 | 1.58 | 0.59 | 1.88 | 3.06 | 0.72 | 2.69 | 0.38 | cathepsin C; Ctsc | AA144887 | dipeptidyl- | the cathepsins are | 7 D3-E1.1 | Proteolytic | | |
| | | | | | | | | | | | | | peptidase 1 | mammalian lysosomal | | | | |
| | | | | | | | | | | | | | precursor (ec | cysteine proteinascs. | | | | |
| | | | | | | | | | | | | | 3.4.14.1) (dpp-i) | they play an important | | | | |
| | | | | | | | | | | | | | (dppi) (cathepsin | role in protein | | | | |
| | | | | | | | | | | | | | c) (csthepsin j) | catabolism within the | | | | |
| | | | | | | | | | | | | | (dipeptidyl | cell, and may be | | | | |
| | | | | | | | | | | | | | transferase). | involved in tumor | | | | |
| | | | | | | | | | | | | | | metastasis | | | | |
| AA184116 | 1.00 | 1.05 | 0.26 | 0.59 | 0.78 | 1.84 | 2.56 | 0.58 | 2.65 | 0.34 | homolog of | AA184116 | | function: f-actin cross- | | Structural | | |
| | | | | | | | | | | | Alpha-actinin | | | linking protein which | | Protein | | |
| | | | | | | | | | | | (human) | | | is thought to anchor | | | | |
| | | | | | | | | | | | | | | actin to a variety of | | | | |
| | | | | | | | | | | | | | | intracellular | | | | |
| | | | | | | | | | | | | | | structures. this is a | | | | |
| | | | | | | | | | | | | | | bundling protein | | | | |
| U88566 | 1.00 | −1.29 | −1.69 | 1.36 | 0.35 | 2.47 | 3.39 | 0.76 | 2.64 | 0.25 | secreted frizzled- | U88566 | | likely involved in wnt | 8 9.5 cM | Cytokine | | |
| | | | | | | | | | | | related sequence | | | binding and signal | | | | |
| | | | | | | | | | | | protein 1; Sfrp1 | | | transduction, deleted | | | | |
| | | | | | | | | | | | | | | in breast carcinomas | | | | |
| AA475191 | 1.00 | −1.11 | 1.12 | 0.68 | 2.10 | 0.47 | 3.44 | 0.86 | 2.64 | 0.25 | cyclin-dependent | AA475191 | | | | Signal | | |
| | | | | | | | | | | | kinase regulatory | | | | | Transduction | | |
| | | | | | | | | | | | subunit 1: Cks1 | | | | | | | |
| ET61664 | 1.00 | −1.03 | −0.49 | 1.06 | 0.19 | 1.69 | 2.98 | 0.11 | 2.63 | 0.42 | Fe receptor, IgG, | ET61664 | low affinity | is a receptor for the fc | 1 92.3 cM | Receptor | | |
| | | | | | | | | | | | low affinity IIb; | | immunoglobulin | region of complexed | | | | |
| | | | | | | | | | | | Fcgr2b | | gamma fc region | immunoglobulins | | | | |
| | | | | | | | | | | | | | receptor ii | gamma. low affinity | | | | |
| | | | | | | | | | | | | | precursor | receptor. involved in a | | | | |
| | | | | | | | | | | | | | (fc- gamma rii) | variety of effector and | | | | |
| | | | | | | | | | | | | | (fcrii) (igg fc | regulatory functions | | | | |
| | | | | | | | | | | | | | receptor ii beta) | such as phagocytosis | | | | |
| | | | | | | | | | | | | | (fc gamma | of antigen-antibody | | | | |
| | | | | | | | | | | | | | receptor iib) | complexes from the | | | | |
| | | | | | | | | | | | | | (fcgammariib). | circulation and | | | | |
| | | | | | | | | | | | | | | modulation of | | | | |
| | | | | | | | | | | | | | | antibody production | | | | |
| | | | | | | | | | | | | | | bu b-cells. isoforms | | | | |
| | | | | | | | | | | | | | | iib1 and iib1′ form | | | | |
| | | | | | | | | | | | | | | caps but fail to | | | | |
| | | | | | | | | | | | | | | mediate endocytosis or | | | | |
| X15592 | 1.00 | 1.41 | 1.00 | 0.36 | 1.39 | 0.16 | 2.95 | 0.38 | 2.62 | 0.33 | cytotoxic T | X15592 | ct1a-2-beta | not known, expressed | 13 42.0 | | | |
| | | | | | | | | | | | lymphocyte- | | protein precursor | in activated t-cell. | cM | | | |
| | | | | | | | | | | | associated protein | | (fragment). | | | | | |
| | | | | | | | | | | | 2 beta: Ct1a2b | | | | | | | |
| AA500688 | 1.00 | −1.07 | 1.04 | 0.63 | 1.99 | 0.48 | 2.68 | 0.47 | 2 62 | 0.21 | EST; Unknown | AA500688 | | | | EST; | | |
| | | | | | | | | | | | | | | | | Unknown | | |
| M14215 | 1.00 | −1.13 | −0.86 | 1.07 | 0.23 | 1.57 | 2.64 | 0.30 | 2.61 | 0.53 | Fe receptor, IgG, | M14215 | low affinity | receptor for the fe | 1 92.3 cM | Receptor | | |
| | | | | | | | | | | | low affinity III; | | immunoglobin | region of complexed | | | | |
| | | | | | | | | | | | Fcgr3 | | gamma fe region | immunoglobulins | | | | |
| | | | | | | | | | | | | | receptor iii | gamma. low affinity | | | | |
| | | | | | | | | | | | | | precursor (igg fc | receptor. | | | | |
| | | | | | | | | | | | | | receptor iii) (fc- | | | | | |
| | | | | | | | | | | | | | gamma riii) (fciii) | | | | | |
| AA212971 | 1.00 | −2.70 | 0 35 | 0.65 | 0.63 | 1.84 | 2.80 | 0.19 | 2.59 | 0.23 | lipopolysaceha- | AA212971 | lipopolysaccha- | binds to the lipid a | 2 83.0 cM | | | |
| | | | | | | | | | | | ride binding | | ride-binding | moiety of bacterial | | | | |
| | | | | | | | | | | | protein; Lbp | | protein precursor | lipopolysaccharides | | | | |
| | | | | | | | | | | | | | (1bp). | (1ps), a glycolipid | | | | |
| | | | | | | | | | | | | | | present in the outer | | | | |
| | | | | | | | | | | | | | | membrane of all gram- | | | | |
| | | | | | | | | | | | | | | negative bacteria. the | | | | |
| | | | | | | | | | | | | | | 1bp/1ps complex seems | | | | |
| | | | | | | | | | | | | | | to interact with the | | | | |
| | | | | | | | | | | | | | | cd14 receptor | | | | |
| J04694 | 1.00 | −1.84 | −0.92 | 0.75 | 0.39 | 1.77 | 2.03 | 0.40 | 2.58 | 0.14 | procollagen, type | J04694 | collagen alpha | type iv collagen forms | 8 5.0 cM | ECM | | |
| | | | | | | | | | | | IV, alpha 1; | | 1(iv) chain | the collagenous matrix | | (Matrix | | |
| | | | | | | | | | | | Col4a1 | | precursor. | of the basement | | Prot) | | |
| | | | | | | | | | | | | | | membrane, an | | | | |
| | | | | | | | | | | | | | | extracellular lamina | | | | |
| | | | | | | | | | | | | | | closely applied to the | | | | |
| | | | | | | | | | | | | | | basal surface of | | | | |
| | | | | | | | | | | | | | | epithelium and also | | | | |
| | | | | | | | | | | | | | | occurring in other | | | | |
| | | | | | | | | | | | | | | tissues | | | | |
| U55060 | 1.00 | −1.11 | −0.14 | 0.95 | 0.46 | 2.02 | 2.12 | 0.36 | 2.58 | 0.19 | lectin, galactose | U55060 | galectin-9. | binds galactosides. | | | | |
| | | | | | | | | | | | binding, soluble | | | may play a role in | | | | |
| | | | | | | | | | | | 9; Lgals9 | | | thymocyte- epithelial | | | | |
| | | | | | | | | | | | | | | interactions relevent to | | | | |
| | | | | | | | | | | | | | | the biology of the | | | | |
| | | | | | | | | | | | | | | thymus. | | | | |
| J05020 | 1.00 | −1.25 | 0.10 | 0.58 | 0.47 | 1.92 | 2.51 | 0.11 | 2.57 | 0.22 | Fc receptor, IgE, | J05020 | high affinity | the high affinity ige | 1 93.3 cM | Cell Surface | | |
| | | | | | | | | | | | high affinity 1, | | immunoglobulin | binding receptor | | Protein | | |
| | | | | | | | | | | | gamma | | epsilon receptor | (fc\epsilonri) is found | | | | |
| | | | | | | | | | | | polypeptide; | | gamma-subunit | exclusively on mast | | | | |
| | | | | | | | | | | | Fcer1 g | | precursor (fceri) | cells and basophils. | | | | |
| | | | | | | | | | | | | | | (ige fc receptor, | | | | |
| | | | | | | | | | | | | | | gamma-subunit) (fc- | | | | |
| | | | | | | | | | | | | | | epsilon rigamma) | | | | |
| AA023914 | 1.00 | 2.46 | 0.98 | 0.71 | 1.88 | 0.37 | 2.22 | 0.36 | 2 56 | 0.17 | | AA023914 | | | | | | |
| U06119 | 1.00 | −1.00 | 0.51 | 0.62 | 1.60 | 0.49 | 2.18 | 0.08 | 2.56 | 0.29 | cathepsin H; Ctsh | U06119 | cathepsin h | activation of | 9 50.0 cM | Proteolytic | | |
| | | | | | | | | | | | | | precursor (cc | macrophages by | | | | |
| | | | | | | | | | | | | | 3.4.22.16) | gamma-interferon | | | | |
| | | | | | | | | | | | | | (cathepsin b3) | induces expression of | | | | |
| | | | | | | | | | | | | | (cathepsin ba). | major | | | | |
| | | | | | | | | | | | | | | histocompatibility | | | | |
| | | | | | | | | | | | | | | complex (mhc) class ii | | | | |
| | | | | | | | | | | | | | | genes. an increase in | | | | |
| | | | | | | | | | | | | | | cathepsin h, encoded | | | | |
| | | | | | | | | | | | | | | in the mouse by ctsh, | | | | |
| | | | | | | | | | | | | | | is also induced by | | | | |
| | | | | | | | | | | | | | | gamma-interferon and | | | | |
| D38162 | 1.00 | −2.75 | −1.55 | 0.62 | −0.14 | 1.74 | 0.58 | 1.10 | 2.54 | 0.31 | procollagen, type | D38162 | collagen alpha | may play an important | 3 53.1 cM | ECM | | |
| | | | | | | | | | | | XI, alpha 1; | | 1(xi) chain | role in fibrillogenesis | | (Matrix | | |
| | | | | | | | | | | | Col11a1 | | precursor. | by controlling lateral | | Prot) | | |
| | | | | | | | | | | | | | | growth of collagen ii | | | | |
| | | | | | | | | | | | | | | fibrils. | | | | |
| AA108054 | 1.00 | −1.40 | −0 41 | 0.50 | 1.29 | 0.21 | 1.78 | 0.28 | 2.53 | 0.24 | serine protease | AA108054 | | | 13 16.0 | | | |
| | | | | | | | | | | | inhibitor 6; Spi6 | | | | cM | | | |
| U56819 | 1.00 | −1.28 | 0.64 | 0.60 | 0.83 | 1.98 | 4.27 | 0.29 | 2.53 | 0.17 | chemokine | U56819 | c—c chemokine | receptor for the mcp-1 | 9 72.0 cM | | | |
| | | | | | | | | | | | (C—C) receptor | | receptor type 2 | (je), mcp-3(fic) and | | | | |
| | | | | | | | | | | | 2; Cmkbr2 | | (c—c ckr-2) | mcp-5 chemokines. | | | | |
| | | | | | | | | | | | | | (cc-ckr-2) (ccr-2) | transduces a signal by | | | | |
| | | | | | | | | | | | | | (ccr2) (je/fic | increasing the | | | | |
| | | | | | | | | | | | | | receptor) (mcp-1 | intracellular calcium | | | | |
| | | | | | | | | | | | | | receptor). | ions level | | | | |
| Msa.16995.0 | 1.00 | −1.11 | −2.58 | 2.64 | −0.12 | 1.95 | 3.20 | 0.17 | 2.52 | 0.46 | arachidonate 5- | W83564 | | seems to be required | | Intracellular | | |
| | | | | | | | | | | | lipoxygenase | | | for the activation of 5- | | Protein | | |
| | | | | | | | | | | | activating protein | | | lo(5-lipoxygenase). | | | | |
| | | | | | | | | | | | | | | flap could play an | | | | |
| | | | | | | | | | | | | | | essential role in the | | | | |
| | | | | | | | | | | | | | | transfer of arachidonic | | | | |
| | | | | | | | | | | | | | | acid tp 5-lo. flap binds | | | | |
| | | | | | | | | | | | | | | to mk-886, a | | | | |
| | | | | | | | | | | | | | | compound that blocks | | | | |
| | | | | | | | | | | | | | | the biosynthesis of | | | | |
| | | | | | | | | | | | | | | leukotrienase | | | | |
| Msa.88.0 | 1.00 | −2.30 | −4.15 | 2.92 | 0.09 | 2.23 | 1.91 | 0.28 | 2.52 | 0.21 | osteoblast specific | D13664 | | preferentially | | Extracellular | | |
| | | | | | | | | | | | factor 2; OSF-2 | | | expressed in | | Protein | | |
| | | | | | | | | | | | | | | periosteum and | | | | |
| | | | | | | | | | | | | | | periodontal ligament. | | | | |
| | | | | | | | | | | | | | | involved in cell | | | | |
| | | | | | | | | | | | | | | adhesion, highly | | | | |
| | | | | | | | | | | | | | | homologous to betaig- | | | | |
| | | | | | | | | | | | | | | h3, a molecule | | | | |
| | | | | | | | | | | | | | | induced by | | | | |
| | | | | | | | | | | | | | | transforming growth | | | | |
| | | | | | | | | | | | | | | factor beta (tgf-beta) | | | | |
| | | | | | | | | | | | | | | that promotes the | | | | |
| | | | | | | | | | | | | | | adhesion and | | | | |
| ET62844 | 1.00 | −1.11 | 0.55 | 0.84 | 1.46 | 0.45 | 2.77 | 0.34 | 2.52 | 0.33 | paired-Ig-like | ET62844 | | | 7 | Receptor | | |
| | | | | | | | | | | | receptor A10, | | | | | | | |
| | | | | | | | | | | | paired-Ig-like | | | | | | | |
| | | | | | | | | | | | receptor A6; | | | | | | | |
| | | | | | | | | | | | Pira10.Pira6 | | | | | | | |
| M33203 | 1.00 | 1.33 | −0.49 | 1.58 | 1.69 | 2.85 | 3.74 | 1.06 | 2.50 | 0.63 | heme oxygenase | M33203 | heme oxygenase | heme oxygenase | 8 C1 | Intracellular | | |
| | | | | | | | | | | | (decycling) 1; | | 1 (ec 1.14.99.3) | catalyzes the | | Protein | | |
| | | | | | | | | | | | Hmox1 | | (ho-1) (p32 | degradation of heme | | | | |
| | | | | | | | | | | | | | protein). | into biliverdin, carbon | | | | |
| | | | | | | | | | | | | | | monooxide, and iron, | | | | |
| | | | | | | | | | | | | | | two forms of this | | | | |
| | | | | | | | | | | | | | | enzyme, heme | | | | |
| | | | | | | | | | | | | | | oxygenase-1 and -2, | | | | |
| | | | | | | | | | | | | | | have been identified; | | | | |
| | | | | | | | | | | | | | | only heme oxygenase- | | | | |
| | | | | | | | | | | | | | | 1 is subject to | | | | |
| | | | | | | | | | | | | | | induction by heme, | | | | |
| D17630 | 1.00 | −1.27 | 0.33 | 0.56 | 0.10 | 1.13 | 2.98 | 0.37 | 2.49 | 1.24 | chemokine | D17630 | high affinity | receptor to interleukin- | 1 40.0 cM | | | |
| | | | | | | | | | | | (C—X—C) | | interleukin-8 | 8, which is a powerful | | | | |
| | | | | | | | | | | | receptor 2; | | receptor b | neutrophils | | | | |
| | | | | | | | | | | | Cmkar2 | | (i1-8r b) (cxcr-2) | chemotacvtic factor, | | | | |
| | | | | | | | | | | | | | (gro/mgsa | binding of i1-8 to the | | | | |
| | | | | | | | | | | | | | receptor). | receptor causes | | | | |
| | | | | | | | | | | | | | | neutrophils. this | | | | |
| | | | | | | | | | | | | | | response is mediated | | | | |
| | | | | | | | | | | | | | | via a g-protein that | | | | |
| | | | | | | | | | | | | | | activate a | | | | |
| | | | | | | | | | | | | | | phosphatidylmositol- | | | | |
| | | | | | | | | | | | | | | calcium second | | | | |
| | | | | | | | | | | | | | | messenger system. this | | | | |
| | | | | | | | | | | | | | | receptor binds to i1-8 | | | | |
| | | | | | | | | | | | | | | with a high affinity | | | | |
| | | | | | | | | | | | | | | and to gro/mgsa and | | | | |
| | | | | | | | | | | | | | | nap-2 also with a high | | | | |
| U41765 | 1.00 | −1.40 | 0.41 | 0.73 | 0.60 | 1.70 | 2.85 | 0.58 | 2.48 | 0.16 | a disintegrin and | U41765 | | | 8 8.0 cM | | | |
| | | | | | | | | | | | metalloproteinase | | | | | | | |
| | | | | | | | | | | | domain 9 (meltrin | | | | | | | |
| | | | | | | | | | | | gamma); Adam9 | | | | | | | |
| Msa.1376.0 | 1.00 | 1.08 | −2.10 | 1.22 | 0.19 | 1.46 | 1.88 | 0.21 | 2.48 | 0.09 | secreted | X16151 | osteopontin | binds tightly to | 5 56.0 cM | Cytokine | | |
| | | | | | | | | | | | phosphoprotein 1; | | precursor (bone | hydroxyapatite. | | | | |
| | | | | | | | | | | | Spp1 | | sialoprotein 1) | appears to form an an | | | | |
| | | | | | | | | | | | | | (minopontin) | integral part of the | | | | |
| | | | | | | | | | | | | | (early t | mineralized matrix. | | | | |
| | | | | | | | | | | | | | lymphocyte | probably important to | | | | |
| | | | | | | | | | | | | | activation 1 | cell-matrix interaction. | | | | |
| | | | | | | | | | | | | | protein) (secreted | | | | | |
| | | | | | | | | | | | | | phosphoprolein 1) | | | | | |
| | | | | | | | | | | | | | (spp-1) (2ar) | | | | | |
| | | | | | | | | | | | | | (calcium oxalate | | | | | |
| | | | | | | | | | | | | | crystal growth | | | | | |
| | | | | | | | | | | | | | inhibitor protein) | | | | | |
| U92437 | 1.00 | −1.85 | 1.71 | 0.67 | 2.06 | 0.03 | 2.72 | 0.35 | 2.48 | 0.28 | phosphatase and | U92437 | protein-tyrosine | potential tumor | 19 24.5 | | | |
| | | | | | | | | | | | tensin homolog; | | phosphatase pten | suppressor. active as a | cM | | | |
| | | | | | | | | | | | Pten | | (cc 3.1.3.48) | phosphatase on | | | | |
| | | | | | | | | | | | | | (mutated in | tyrosine, serine and | | | | |
| | | | | | | | | | | | | | multiple | threonine residues. | | | | |
| | | | | | | | | | | | | | advanced cancers | | | | | |
| AA667371 | 1.00 | −2.99 | −1.56 | 0.83 | −0.40 | 2.70 | 0.68 | 0.93 | 2.42 | 0.25 | Unknown | AA667371 | | no match on blast | | EST; | | |
| | | | | | | | | | | | | | | search 1/99. | | Unknown | | |
| Msa.7498.0 | 1.00 | 1.38 | −0.01 | 1.13 | 0.49 | 1.77 | 2.90 | 0.20 | 2.41 | 0.37 | growth arrest and | AA138777 | growth arrest and | plays an important | | Regulartory | | |
| | | | | | | | | | | | DNA-damage- | | dna-damage- | role in negative | | | | |
| | | | | | | | | | | | inducible, | | inducible protein | growth control, | | | | |
| | | | | | | | | | | | gamma; Gadd45g | | gadd45 gamma | including both growth | | | | |
| | | | | | | | | | | | | | (cytokine | suppression and | | | | |
| | | | | | | | | | | | | | responsive | apoptosis. | | | | |
| | | | | | | | | | | | | | protein cr6). | | | | | |
| S74567 | 1.00 | −1.41 | 3.30 | 0.41 | 2.84 | 1.39 | 2.88 | 0.80 | 2.39 | 1.21 | avian | S74567 | transcription | the c-maf interaction | 8 61.0 cM | | | |
| | | | | | | | | | | | musculoaponeu- | | factor maf2 | site was mapped to the | | | | |
| | | | | | | | | | | | rotic fibrosarcoma | | (proto-oncogene | sequence 5′- | | | | |
| | | | | | | | | | | | (v-mat) AS42 | | c-maf). | [gt]g[gc]n[gt]nctcagnn. | | | | |
| | | | | | | | | | | | oncogene | | | 3′ in the 17 promotor. it | | | | |
| | | | | | | | | | | | homolog; Maf | | | may interact with | | | | |
| | | | | | | | | | | | | | | additional basic-zipper | | | | |
| | | | | | | | | | | | | | | proteins that | | | | |
| | | | | | | | | | | | | | | determine a subtype of | | | | |
| | | | | | | | | | | | | | | maf-responsive | | | | |
| | | | | | | | | | | | | | | element binding | | | | |
| AA238081 | 1.00 | −1.19 | −1.01 | 1.19 | 0.78 | 2.13 | 2.04 | 0.37 | 2.39 | 0.24 | complement | AA238081 | | | 6 | | | |
| | | | | | | | | | | | component 1, r | | | | | | | |
| | | | | | | | | | | | subcomnonent: | | | | | | | |
| | | | | | | | | | | | C1r | | | | | | | |
| W53443 | 1.00 | 1.39 | −1.66 | 1.86 | 0.32 | 1.35 | 1.74 | 0.34 | 2.38 | 0.13 | GENESEQN: | W53443 | | also a good match to | | EST; | | |
| | | | | | | | | | | | V34267 Human | | | human clone 2491 | | Unknown | | |
| | | | | | | | | | | | secreted protein | | | (af131781) (both are | | | | |
| | | | | | | | | | | | gene 58 clone | | | 89%) | | | | |
| | | | | | | | | | | | HSSEP68. | | | | | | | |
| L28177 | 1.00 | 1.22 | −0.67 | 1.07 | 0.03 | 1.44 | 5.51 | 0.19 | 2.36 | 0.44 | DNA-damage | L28177 | growth arrest and | binds to proliferating | 3 70.5 cM | Intracellular | | |
| | | | | | | | | | | | inducible | | dna-damage- | cell nuclear antigen | | Protein | | |
| | | | | | | | | | | | transcript 1; | | inducible protein | might affect pena | | | | |
| | | | | | | | | | | | Ddit1 | | gadd45. | interaction with some | | | | |
| | | | | | | | | | | | | | | edk (cell division | | | | |
| | | | | | | | | | | | | | | protein kinase) | | | | |
| | | | | | | | | | | | | | | complexes; stimulates | | | | |
| | | | | | | | | | | | | | | dna excision repair in | | | | |
| | | | | | | | | | | | | | | vitro and inhibits entry | | | | |
| | | | | | | | | | | | | | | of cells into r phase | | | | |
| Msa 928.0 | 1.00 | −1.07 | −1.99 | 1.35 | 0.25 | 1.47 | 1.88 | 0.09 | 2.35 | 0.18 | myristoylated | M60474 | myristoylated | marcks is the most | 10 22.0 | Structural | | |
| | | | | | | | | | | | alanine rich | | alanine-rich c- | prominent cellular | cM | Protein | | |
| | | | | | | | | | | | protein kinase C | | kinase substrate | substrate for protein | | | | |
| | | | | | | | | | | | substrate Macs | | (marcks). | kinase c. this protein | | | | |
| | | | | | | | | | | | | | | binds calmodulin, | | | | |
| | | | | | | | | | | | | | | actin, and synapsin. | | | | |
| | | | | | | | | | | | | | | marcks is a | | | | |
| | | | | | | | | | | | | | | filamentous (f) actin | | | | |
| | | | | | | | | | | | | | | cross-linking protein | | | | |
| AA475111 | 1.00 | −1.27 | 1.20 | 0.45 | 1.54 | 0.12 | 2.49 | 0.30 | 2.31 | 0.19 | heterogeneous | AA475111 | | | | | | |
| | | | | | | | | | | | nuclear | | | | | | | |
| | | | | | | | | | | | ribonucle- | | | | | | | |
| | | | | | | | | | | | protein D | | | | | | | |
| AA536849 | 1.00 | −1.97 | 0.31 | 0.73 | 3.28 | 1.85 | 2.32 | 0.71 | 2.31 | 0.51 | EST; Unknown | AA536849 | | | | EST; | | |
| | | | | | | | | | | | | | | | | Unknown | | |
| C81524 | 1.00 | −1.20 | 2.02 | 0.85 | 1.09 | 2.55 | 1.96 | 1.84 | 2.31 | 1.29 | | C81524 | | | | | | |
| AA170245 | 1.00 | 1.08 | 0.79 | 0.45 | 0.60 | 1.69 | 2.32 | 0.10 | 2.30 | 0.18 | | AA170245 | | | | | | |
| AF013262 | 1.00 | −2.69 | −0.93 | 0.72 | 1.19 | 2.74 | 2.01 | 0.60 | 2.30 | 0.72 | lumican; Lum | AF013262 | lumican precursor | leucine-rich | 10 61.0 | ECM | | |
| | | | | | | | | | | | | | (lum) (keratin | proteoglycan with | cM | (Matrix | | |
| | | | | | | | | | | | | | sulfate | keratin sulfate side | | Prot) | | |
| | | | | | | | | | | | | | protcoglycan). | chains, a major | | | | |
| | | | | | | | | | | | | | | component of cornea, | | | | |
| | | | | | | | | | | | | | | dermal, and muscle | | | | |
| | | | | | | | | | | | | | | connective tissues. | | | | |
| | | | | | | | | | | | | | | regulation of collagen | | | | |
| | | | | | | | | | | | | | | assembly into fibrils in | | | | |
| | | | | | | | | | | | | | | various connective | | | | |
| | | | | | | | | | | | | | | tissues. lumican is | | | | |
| | | | | | | | | | | | | | | necessary in the | | | | |
| AF020313 | 1.00 | −1.73 | 0.47 | 0.68 | 1.07 | 2.46 | 3.40 | 1.07 | 2.30 | 0.16 | amyloid beta (A4) | AF020313 | | | | Extracellular | | |
| | | | | | | | | | | | precursor protein- | | | | | Protein | | |
| | | | | | | | | | | | binding, family | | | | | | | |
| | | | | | | | | | | | B, member 1 | | | | | | | |
| | | | | | | | | | | | interacting | | | | | | | |
| | | | | | | | | | | | protcin; Apbb1ip- | | | | | | | |
| | | | | | | | | | | | pending | | | | | | | |
| Msa.22488.0 | 1.00 | 1.08 | −1.88 | 1.54 | 0.27 | 2.19 | 2.75 | 0.57 | 2.30 | 0.22 | cathepsin S; Ctss | AA146437 | | the cathepsins are | 3 42.7 cM | Proteolytic | | |
| | | | | | | | | | | | | | | mammalian lysosomal | | | | |
| | | | | | | | | | | | | | | cysteine proteinases. | | | | |
| | | | | | | | | | | | | | | they play an important | | | | |
| | | | | | | | | | | | | | | role in protein | | | | |
| | | | | | | | | | | | | | | catabolism within the | | | | |
| | | | | | | | | | | | | | | cell, and may be | | | | |
| | | | | | | | | | | | | | | involved in tumor | | | | |
| | | | | | | | | | | | | | | metastasis | | | | |
| AF004666 | 1.00 | 1.09 | 0.15 | 0.48 | 0.11 | 1.17 | 2.68 | 0.32 | 2.27 | 0.40 | solute carrier | AF004666 | sodium/calcium | rapidly transports ca2+ | 17 48.0 | | | |
| | | | | | | | | | | | family 8 | | exchanger 1 | during excitation- | cM | | | |
| | | | | | | | | | | | (sodium/calcium | | precursor | contraction coupling. | | | | |
| | | | | | | | | | | | exchanger), | | (na+/ca2+- | ca(2+) is extruded | | | | |
| | | | | | | | | | | | member 1; Sle8a1 | | exchange | from the celll during | | | | |
| | | | | | | | | | | | | | protein 1). | relaxation so as to | | | | |
| | | | | | | | | | | | | | | prevent overloading of | | | | |
| | | | | | | | | | | | | | | intracellular stores | | | | |
| Msa.1171.0 | 1.00 | 0.63 | −0.50 | 0.61 | 0.72 | 2.71 | −0.69 | 1.03 | 2.27 | 2.79 | keratin-associated | M37760 | | | | | | |
| | | | | | | | | | | | protein 5-4; | | | | | | | |
| | | | | | | | | | | | Krtap5-4 | | | | | | | |
| Msa.683.0 | 1.00 | −1.85 | −0.89 | 1.02 | 0.31 | 1.63 | 2.14 | 0.05 | 2.25 | 0.17 | lectin, galactose | W13002 | galectin-1 (beta- | postimplantation, | 15 44.9 | Other | | |
| | | | | | | | | | | | binding, soluble | | galactoside- | 1gals1 is expressed in | cM | | | |
| | | | | | | | | | | | 1; Lga1s1 | | binding lectin | somite myotomes, | | | | |
| | | | | | | | | | | | | | 1-14-i) (lactose- | suggesting a role in | | | | |
| | | | | | | | | | | | | | binding lectin 1) | muscle development. | | | | |
| | | | | | | | | | | | | | (s-lac lectin 1) | this protein binds beta- | | | | |
| | | | | | | | | | | | | | (galaptin) (14 kda | galactoside, its | | | | |
| | | | | | | | | | | | | | lectin) | physiological function | | | | |
| | | | | | | | | | | | | | | is not yet known. it | | | | |
| | | | | | | | | | | | | | | may act as an | | | | |
| | | | | | | | | | | | | | | autocrine negative | | | | |
| | | | | | | | | | | | | | | growth factor that | | | | |
| Msa.5619.0 | 1.00 | −1.16 | −3.48 | 2.53 | −0.07 | 2.03 | 2.48 | 0.33 | 2.22 | 0 22 | protease, | AA000961 | | a cysteine | | Proteolytic | | |
| | | | | | | | | | | | cysteine, 1; | | | endopeptidase. | | | | |
| | | | | | | | | | | | Prsc1 | | | legumain was found in | | | | |
| | | | | | | | | | | | | | | all mouse tissues | | | | |
| | | | | | | | | | | | | | | examined, but was | | | | |
| | | | | | | | | | | | | | | particularly abundant | | | | |
| | | | | | | | | | | | | | | in kidney and | | | | |
| | | | | | | | | | | | | | | placenta, the | | | | |
| | | | | | | | | | | | | | | distribution in | | | | |
| | | | | | | | | | | | | | | subcellular fractions | | | | |
| | | | | | | | | | | | | | | of mouse and rat | | | | |
| | | | | | | | | | | | | | | kidney showed a | | | | |
| | | | | | | | | | | | | | | lysosomal | | | | |
| AA119603 | 1.00 | 1.13 | 1.06 | 0.58 | 1.58 | 0.16 | 2.17 | 0.13 | 2.22 | 0.30 | | AA119603 | | | | | | |
| D84391 | 1.00 | −1.58 | 1.92 | 0.91 | 2.05 | 0.87 | 1.83 | 1.73 | 2.21 | 1.32 | Li repeat, Tf | D84391 | | | | EST; | | |
| | | | | | | | | | | | subfamily, | | | | | Unknown | | |
| | | | | | | | | | | | member 14,L1 | | | | | | | |
| | | | | | | | | | | | repeat, Tf | | | | | | | |
| | | | | | | | | | | | subfamily, | | | | | | | |
| | | | | | | | | | | | member 29; | | | | | | | |
| | | | | | | | | | | | L1Md | Tf14,L1 | | | | | | |
| | | | | | | | | | | | Md-Tf29 | | | | | | | |
| X16874 | 1.00 | −1.38 | −1.89 | 2.31 | 0.25 | 1.88 | 2.10 | 0.00 | 2.17 | 0.18 | complement | X16874 | complement c1q | the primary humoral | 4 66.1 cM | Hemostasis | | |
| | | | | | | | | | | | component 1, q | | subcomponent, b | mediator of antigen- | | | | |
| | | | | | | | | | | | subcomponent, | | chain precursor. | antibody reactions is | | | | |
| | | | | | | | | | | | beta polypeptide; | | | the complement (c) | | | | |
| | | | | | | | | | | | C1qb | | | system. | | | | |
| AF022992 | 1.00 | 4.47 | 1 39 | 0.28 | 0.21 | 1.33 | 1.55 | 1.17 | 2.16 | 0.30 | period homolog | AF022992 | per-hexamer | circadian regulator | 11 B | Other | | |
| | | | | | | | | | | | (Drosophila); Per | | repeat protein 5., | that may act as a | | | | |
| | | | | | | | | | | | | | period circadian | transcription factor. | | | | |
| | | | | | | | | | | | | | protein 1 | behaves as a negative | | | | |
| | | | | | | | | | | | | | (circadian | element in circadian | | | | |
| | | | | | | | | | | | | | pacemaker | transcriptional loop | | | | |
| | | | | | | | | | | | | | protein | | | | | |
| | | | | | | | | | | | | | rigui)(mner)(m- | | | | | |
| U69135 | 1.00 | 1.15 | −7.17 | 7.77 | 1.42 | 0.28 | 2.76 | 0.71 | 2.16 | 0.11 | uncoupling | U69135 | mitochondrial | ucp are mitochondrial | 7 50.0 cM | Intracellular | | |
| | | | | | | | | | | | protein 2, | | uncoupling | transporter proteins | | Protein | | |
| | | | | | | | | | | | mitochondrial; | | protein 2 (ucp 2) | that create proton | | | | |
| | | | | | | | | | | | Ucp2 | | (ucph). | leaks across the inner | | | | |
| | | | | | | | | | | | | | | mitochondrial | | | | |
| | | | | | | | | | | | | | | membrane, thus | | | | |
| | | | | | | | | | | | | | | uncoupling oxydative | | | | |
| | | | | | | | | | | | | | | phosphorylation from | | | | |
| | | | | | | | | | | | | | | atp synthesis, as a | | | | |
| | | | | | | | | | | | | | | result, energy is | | | | |
| | | | | | | | | | | | | | | dissipated in the form | | | | |
| | | | | | | | | | | | | | | of heat | | | | |
| Msa.4530.0 | 1.00 | 1.34 | 0.13 | 0.56 | 1.33 | 0.21 | 2.22 | 0.10 | 2.16 | 0.41 | EST; region of | AA106931 | | | | EST; | | |
| | | | | | | | | | | | homolgy to | | | | | Unknown | | |
| | | | | | | | | | | | GENESEQN: | | | | | | | |
| | | | | | | | | | | | Z77537 Human | | | | | | | |
| | | | | | | | | | | | ovarian tumor | | | | | | | |
| | | | | | | | | | | | cDNA library | | | | | | | |
| | | | | | | | | | | | derived FST | | | | | | | |
| | | | | | | | | | | | fragment 88 | | | | | | | |
| Msa.34975.0 | 1.00 | 3.11 | 1.12 | 0.58 | 1.92 | 0.83 | 2.57 | 0.42 | 2.14 | 0.22 | eukaryotic | AA118716 | eukaryotic | eif-2 functions in the | Y | Regulatory | | |
| | | | | | | | | | | | translation | | translation | early steps of protein | | | | |
| | | | | | | | | | | | initiation factor 2, | | initiation factor 2 | synthesis by forming a | | | | |
| | | | | | | | | | | | subunit 3, | | gamma subunit y- | ternary complex with | | | | |
| | | | | | | | | | | | structural gene V- | | linked (eif | gtp and initiator trna. | | | | |
| | | | | | | | | | | | linked; Eit2s3y | | 2-gamma y). | this complex binds to | | | | |
| | | | | | | | | | | | | | | a 4s ribsomal | | | | |
| | | | | | | | | | | | | | | subunit, followed by | | | | |
| | | | | | | | | | | | | | | mrna binding to form | | | | |
| | | | | | | | | | | | | | | a 43s preinitiation | | | | |
| | | | | | | | | | | | | | | complex. junction of | | | | |
| | | | | | | | | | | | | | | the 6s ribosomal | | | | |
| | | | | | | | | | | | | | | subunit to form the 8s | | | | |
| | | | | | | | | | | | | | | initiation complex is | | | | |
| | | | | | | | | | | | | | | preceeded by hydrolysis | | | | |
| | | | | | | | | | | | | | | of the gtp bound to eif- | | | | |
| | | | | | | | | | | | | | | 2 and release of an eif- | | | | |
| | | | | | | | | | | | | | | 2-gdp binary complex. | | | | |
| | | | | | | | | | | | | | | in order for eif-2 to | | | | |
| | | | | | | | | | | | | | | recycle and catalyze | | | | |
| | | | | | | | | | | | | | | another round of | | | | |
| | | | | | | | | | | | | | | initiation, the gdp | | | | |
| | | | | | | | | | | | | | | bound to eif-2 must | | | | |
| | | | | | | | | | | | | | | exchange with gtp by | | | | |
| | | | | | | | | | | | | | | way of a reaction | | | | |
| | | | | | | | | | | | | | | catalyzed by eif-2b (by | | | | |
| AA616077 | 1.00 | −1.88 | −0.15 | 0.53 | 1.56 | 0.52 | 2.53 | 0.27 | 2.13 | 0.17 | | AA616077 | | | | EST; | | |
| | | | | | | | | | | | | | | | | Unknown | | |
| AA607513 | 1.00 | −3.27 | −3.57 | 2.01 | −0.15 | 2.43 | 0.84 | 0.96 | 2.13 | 0.33 | Unknown | AA607513 | | blast analysis of 12/99. | | EST; | bIas: | |
| | | | | | | | | | | | | | | blast analysis of 5/ | | Unknown | | |
| ET62894 | 1.00 | −3.02 | 0.12 | 0.95 | 0.03 | 1.39 | 1.03 | 1.20 | 2.09 | 0.16 | | ET62894 | | | | EST; | | |
| | | | | | | | | | | | | | | | Unknown | | | |
| U31993 | 1.00 | 1.00 | −0.75 | 0.59 | 0.42 | 1.89 | 2.35 | 0.25 | 2.08 | 0.24 | interleukin 17 | U31993 | | | 6 55.2 cM | Receptor | | |
| | | | | | | | | | | | receptor; II17r | | | | | | | |
| Msa.9251.0 | 1.00 | −1.29 | −1.33 | 0.91 | 0.29 | 1.90 | 2.56 | 0.10 | 2.05 | 0.37 | neutrophil | AA050149 | | | 1 76.1 cM | Intracellular | | |
| | | | | | | | | | | | cytosolic | | | | | Protein | | |
| | | | | | | | | | | | factor 2; Ncf2 | | | | | | | |
| Msa.978.0 | 1.00 | −1.00 | −1.30 | 1.35 | 0.07 | 1.36 | 2.07 | 0.05 | 2.04 | 0.24 | moesin; Msn | M86390 | moesin | thought to work as | X | Cell Surface | | |
| | | | | | | | | | | | | | (membrane- | cross-linkers between | | Protein | | |
| | | | | | | | | | | | | | organizing | plasma membranes | | | | |
| | | | | | | | | | | | | | extension spike | and actin-based | | | | |
| | | | | | | | | | | | | | protein). | cytoskeletons. these | | | | |
| | | | | | | | | | | | | | | molecules are | | | | |
| | | | | | | | | | | | | | | involved not only in | | | | |
| | | | | | | | | | | | | | | cytoskeletal | | | | |
| | | | | | | | | | | | | | | organization but also | | | | |
| | | | | | | | | | | | | | | in signal transduction | | | | |
| AA474881 | 1.00 | −1.06 | 1.37 | 0.61 | 1.85 | 0.05 | 2.66 | 0.28 | 2.01 | 0.20 | DNA segment, | AA474881 | | | 1 17.0 cM | | | |
| | | | | | | | | | | | Chr 1, Wayne | | | | | | | |
| | | | | | | | | | | | State | | | | | | | |
| | | | | | | | | | | | University 40, | | | | | | | |
| | | | | | | | | | | | expressed; | | | | | | | |
| AA204590 | 1.00 | −1.39 | 0.50 | 0.54 | 0.11 | 1.23 | 3.51 | 0.74 | 2.01 | 0.31 | EST; Unknown | AA204590 | | | | EST; | | |
| | | | | | | | | | | | | | | | | Unknown | | |
| AA691533 | 1.00 | −7.45 | −1.37 | 0.88 | 0.56 | 2.15 | 0.50 | 0.96 | 2.00 | 0.50 | | AA691533 | | | | EST; | | |
| | | | | | | | | | | | | | | | | Unknown | | |
| Msa.17862.0 | 1.00 | −6 92 | −1.98 | 0.96 | −0.02 | 1.49 | 1.85 | 0.27 | 1.99 | 0.12 | lysyl oxidase-like; | W98413 | | | 9 33.0 cM | | | |
| | | | | | | | | | | | Lox1 | | | | | | | |
| Msa.29217.0 | 1.00 | −3.11 | −1.61 | 3.81 | 1.65 | 0.59 | 2.80 | 0.79 | 1.96 | 0.30 | actin, beta, | AA079937 | | | | Structural | | |
| | | | | | | | | | | | cytoplasmic | | | | | | | |
| Msa.24381.0 | 1.00 | −2.44 | 0.70 | 0.66 | 0.52 | 1.55 | 2.40 | 0.55 | 1.92 | 0.35 | damage specific | W42399 | | a dna binding protein | 19 5.0 cM | Other | | |
| | | | | | | | | | | | DNA binding | | | that binds specifically | | | | |
| | | | | | | | | | | | protein | | | to damaged dna. a | | | | |
| | | | | | | | | | | | 1(127 kDa); Ddb1 | | | defect in binding | | | | |
| | | | | | | | | | | | | | | activity is associated | | | | |
| | | | | | | | | | | | | | | with xeroderma | | | | |
| | | | | | | | | | | | | | | pigmentosum e (xpe) | | | | |
| | | | | | | | | | | | | | | in humans | | | | |
| X57337 | 1.00 | −2.45 | −1.87 | 0.89 | −0.11 | 1.76 | 1.39 | 0.13 | 1.91 | 0.19 | procollagen C- | X57337 | procollagen c- | a glycoprotein that | 5 78.0 cM | Extracellular | | |
| | | | | | | | | | | | proteinase | | proteinase | potentiates enzymatic | | Protein | | |
| | | | | | | | | | | | enhancer | | enhancer protein | cleavage of the type i | | | | |
| | | | | | | | | | | | protein; Pcolce | | precursor (pcpe) | procollagen c- | | | | |
| | | | | | | | | | | | | | (type i | propeptide by bone | | | | |
| | | | | | | | | | | | | | procollagen cooh- | morphogenetic protein- | | | | |
| | | | | | | | | | | | | | terminal | 1 (bmp-1) | | | | |
| | | | | | | | | | | | | | proteinase | | | | | |
| | | | | | | | | | | | | | enhancer) (type 1 | | | | | |
| | | | | | | | | | | | | | procollagen c- | | | | | |
| | | | | | | | | | | | | | proteinase | | | | | |
| | | | | | | | | | | | | | enhancer | | | | | |
| | | | | | | | | | | | | | | protein) (p1d) | | | | |
| Msa.2924.0 | 1.00 | −1.06 | −0.71 | 0.56 | 0.35 | 1.36 | 2.66 | 0.30 | 1.91 | 0.27 | superoxide | X84940 | extracellular | destroys radicals | 5 31.0 cM | | | |
| | | | | | | | | | | | dismutase 3, | | superoxide | which are normally | | | | |
| | | | | | | | | | | | extracellular; | | dismutase | produced within the | | | | |
| | | | | | | | | | | | Sod.3 | | [cu—zn] | cells and are toxic to | | | | |
| | | | | | | | | | | | | | precursor (ec | biological systems. | | | | |
| | | | | | | | | | | | | | 1.15.1.1) | | | | | |
| | | | | | | | | | | | | | (ec-sod). | | | | | |
| AA608277 | 1.00 | 1.72 | 0.73 | 0.50 | 1.21 | 0.03 | 2.36 | 0.18 | 1.91 | 0.42 | | AA608277 | | | | | | |
| U75530 | 1.00 | −1.41 | 3.24 | 1.00 | 0.89 | 2.65 | 1.99 | 3.07 | 1.90 | 1.73 | eukaryotic | U75530 | | | 10 32.0 | | | |
| | | | | | | | | | | | translation | | | | cM | | | |
| | | | | | | | | | | | initiation factor | | | | | | | |
| | | | | | | | | | | | 4E binding | | | | | | | |
| | | | | | | | | | | | protein 2; | | | | | | | |
| | | | | | | | | | | | Eif4cbp2 | | | | | | | |
| M91380 | 1.00 | −2.56 | −2.42 | 1.30 | −0.27 | 1.66 | 1.64 | 0.21 | 1.89 | 0.10 | follistatin-like; | M91380 | follistatin-related | tgfb responsive gene | 16 27.3 | Extracellular | | |
| | | | | | | | | | | | Fst1 | | protein precursor | cloned from an | cM | Protein | | |
| | | | | | | | | | | | | | (tgf-beta- | osteoblastic cell line. | | | | |
| | | | | | | | | | | | | | inducible protein | encodes a protein of | | | | |
| | | | | | | | | | | | | | tsc-36). | 35 kda. the amino acid | | | | |
| | | | | | | | | | | | | | | sequence of tsc-36 | | | | |
| | | | | | | | | | | | | | | protein was found to | | | | |
| | | | | | | | | | | | | | | be similar to | | | | |
| | | | | | | | | | | | | | | follistatin, an activin- | | | | |
| | | | | | | | | | | | | | | binding protein, also | | | | |
| | | | | | | | | | | | | | | similar to the secreted | | | | |
| | | | | | | | | | | | | | | protein rich i | | | | |
| Msa.21961.0 | 1.00 | −2.48 | −0.32 | 0.79 | −0.12 | 1.13 | 1.58 | 0.25 | 1.89 | 0.26 | EST; Unknown | AA030421 | | est | | EST; | | |
| | | | | | | | | | | | | | | | | Unknown | | |
| Z31334 | 1.00 | −2.57 | −3.02 | 1.52 | −0.47 | 1.96 | 0.49 | 0.81 | 1.84 | 0.12 | procollagen, type | Z31334 | collagen alpha | type i collagen, the | 6 0.68 cM | ECM | | |
| | | | | | | | | | | | 1, alpha 2; Cola2 | | 2(i) chain | commonest form, is a | | (Matrix | | |
| | | | | | | | | | | | | | precursor. | fibrillar collagen, | | Prot) | | |
| | | | | | | | | | | | | | | along with types ii, iii, | | | | |
| | | | | | | | | | | | | | | v, and xi | | | | |
| AA176016 | 1.00 | −2.13 | 0 89 | 0.73 | 1.69 | 0.05 | 2.43 | 0.32 | 1.82 | 0.19 | | AA176016 | | | | | | |
| AA510381 | 1.00 | 1.29 | 1.66 | 0.99 | 0.08 | 1.75 | 1.14 | 1.51 | 1.82 | 1.19 | | AA510381 | | | | | | |
| AA146539 | 1.00 | −1.05 | 1.80 | 0.52 | 1.91 | 0.05 | 2.13 | 0.31 | 1.78 | 0.07 | EGF-like repeats | AA146539 | | | | | | |
| | | | | | | | | | | | and discordin | | | | | | | |
| | | | | | | | | | | | 1-like domains | | | | | | | |
| | | | | | | | | | | | 3; Edil3 | | | | | | | |
| Msa.2536.0 | 1.00 | 2.69 | −0.19 | 0.81 | 1.43 | 0.08 | 1.88 | 0.57 | 1.74 | 0.20 | extracellular | X93037 | wdnm1 protein | could have proteinase | | | | |
| | | | | | | | | | | | proteinase | | precursor | inhibiting capacity. | | | | |
| | | | | | | | | | | | inhibitor, | | | | | | | |
| | | | | | | | | | | | Expi | | | | | | | |
| D78188 | 1.00 | −2.03 | 0.04 | 0.66 | 0.34 | 2.02 | 1.82 | 0.41 | 1.74 | 0.26 | granule cell | D78188 | myotrophin (v-1 | potential role in | | | | |
| | | | | | | | | | | | differentiation | | protein) (granule | cerebellar | | | | |
| | | | | | | | | | | | protein; Gcdp | | cell | morphogenesis. may | | | | |
| | | | | | | | | | | | | | differentiation | function in | | | | |
| | | | | | | | | | | | | | protein). | differentiation of | | | | |
| | | | | | | | | | | | | | | cerebellar neurons, | | | | |
| | | | | | | | | | | | | | | particularly of granule | | | | |
| | | | | | | | | | | | | | | cells | | | | |
| X93037 | 1.00 | 3.54 | −0.36 | 0.70 | 1.60 | 0.26 | 1.81 | 0.49 | 1.73 | 0.20 | extracellular | X93037 | wdnm1 protein | could have proteinase | | | | |
| | | | | | | | | | | | proteinase | | precursor. | inhibiting capacity. | | | | |
| | | | | | | | | | | | inhibitor; | | | | | | | |
| | | | | | | | | | | | Expi | | | | | | | |
| W45778 | 1.00 | 4.09 | −1.35 | 1.46 | 0.39 | 2.02 | 0.59 | 0.83 | 1.73 | 0.38 | von Willebrand | W45778 | | vwfbinds to and | | Hemostasis | | |
| | | | | | | | | | | | Factor; vWF; | | | stabilizes coagulation | | | | |
| | | | | | | | | | | | homolog | | | factor vii (cf8) and | | | | |
| | | | | | | | | | | | | | | also mediates | | | | |
| | | | | | | | | | | | | | | interaction between | | | | |
| | | | | | | | | | | | | | | platelets and the blood | | | | |
| | | | | | | | | | | | | | | vessel wall. expressed | | | | |
| | | | | | | | | | | | | | | in endothelial | | | | |
| | | | | | | | | | | | | | | megakaryocytes, | | | | |
| | | | | | | | | | | | | | | stored in platelets | | | | |
| | | | | | | | | | | | | | | alpha-granules and | | | | |
| | | | | | | | | | | | | | | within the weibel- | | | | |
| Msa.2220 0 | 1.00 | −2.16 | 0.07 | 0.79 | 0.03 | 1.17 | 0.67 | 0.91 | 1.71 | 0.21 | procollagen, type | X58251 | collagen alpha | forms the fibrils of | 6 0.68 cM | ECM | | |
| | | | | | | | | | | | I, alpha 2; Cola2 | | 2(i) chain | tendon, ligaments and | | (Matrix | | |
| | | | | | | | | | | | | | precursor. | bones. in bones the | | Prot) | | |
| | | | | | | | | | | | | | | fibrils are mineralized | | | | |
| | | | | | | | | | | | | | | with calcium | | | | |
| | | | | | | | | | | | | | | hydroxyapatite | | | | |
| V01527 | 1.00 | −2.25 | −0.93 | 1.01 | 0.19 | 1.46 | 1.95 | 0.04 | 1.71 | 0.21 | histocompatibility | V01527 | h-2 class ii | this a class ii | 17 18.64 | Cell Surface | | |
| | | | | | | | | | | | 2, class II antigen | | histocompatibility | antigen, i-a-beta. | cM | Protein | | |
| | | | | | | | | | | | A, beta 1; H-Ab1 | | antigen, a-d beta | | | | | |
| | | | | | | | | | | | | | chain precursor | | | | | |
| U79144 | 1.00 | −5.09 | −3.07 | 1.98 | −0.09 | 1.71 | 1.50 | 0.17 | 1.70 | 0.21 | lysyl oxidase-like; | U79144 | | homolog of lysyl | 9 33.0 cM | EST; | | |
| | | | | | | | | | | | Lox1 | | | oxidase which maps to | | Unknown | | |
| | | | | | | | | | | | | | | chr 18 | | | | |
| U64450 | 1.00 | −0.37 | 1.53 | 0.84 | 0.23 | 1.85 | 2 57 | 0.59 | 1.68 | 0.63 | nucleoplasmin 3; | U64450 | | aka nub1 -- encodes a | 19 45.0 | Regulatory | | |
| | | | | | | | | | | | Npm3 | | | protein related to the | cM | | | |
| | | | | | | | | | | | | | | nuclear chaperone | | | | |
| | | | | | | | | | | | | | | phosphoproteins, | | | | |
| | | | | | | | | | | | | | | nucleoplasmin and | | | | |
| | | | | | | | | | | | | | | nucleophosmin | | | | |
| U11541 | 1.00 | −5.90 | −32.12 | 30.53 | −0.45 | 1.59 | −2.26 | 1.89 | 1.68 | 0.31 | bone gamma | U11541 | osteocalcin | a.k.a. as osteocalcin- | 3 42.6 cM | ECM | | |
| | | | | | | | | | | | carhoxyglutamate | | precursor | related protein. bglap- | | (Matrix | | |
| | | | | | | | | | | | protein 1 ,bone | | (gamma-carboxy- | rs1, is a related | | Prot) | | |
| | | | | | | | | | | | gamma- | | glutamic | sequence which may | | | | |
| | | | | | | | | | | | carboxyglulamate | | acid-containing | not to be transcribed. it is | proteii | | | |
| | | | | | | | | | | | protein 2,bone | | protein) (bone | claimed to resemble, | | | | |
| | | | | | | | | | | | gamma- | gla-protein) | in structure and | | | | | |
| | | | | | | | | | | | carboxyglutamate | | (bgp)., | expression pattern, | | | | |
| | | | | | | | | | | | protein, related | | osteocalcin- | nephrocalcin, a | precut | | | |
| | | | | | | | | | | | sequence 1; | | related protein | calcium-binding | | | | |
| | | | | | | | | | | | Bglap-rs1, | | precursor (oc-x) | protein involved in | | | | |
| | | | | | | | | | | | Bglap1,Bglap2 | | (nephrocalcin). | kidney calcium | | | | |
| D50460 | 1.00 | −3.16 | −1.78 | 0.97 | 0.21 | 1.47 | 0.41 | 0.87 | 1.67 | 0.15 | stromal cell | D50460 | pigment | a.k.a. pigment | | Cytokine | | |
| | | | | | | | | | | | derived factor | | epithelium- | epithelium-derived | | | | |
| | | | | | | | | | | | 3; Sdf3 | | derived factor | factor -- a member of | | | | |
| | | | | | | | | | | | | | precursor (pedf) | the serine protease | | | | |
| | | | | | | | | | | | | | (stromal cell- | inhibitor (serpin | | | | |
| | | | | | | | | | | | | | derived factor 3) | superfamily, promotes | | | | |
| | | | | | | | | | | | | | (sdf-3). | survival and/or | | | | |
| | | | | | | | | | | | | | | differentiation of rat | | | | |
| | | | | | | | | | | | | | | cerebellar granule | | | | |
| | | | | | | | | | | | | | | neurons and human | | | | |
| | | | | | | | | | | | | | | retinoblastoma cells in | | | | |
| | | | | | | | | | | | | | | vitro, no inhibitory | | | | |
| C76162 | 1.00 | −2.16 | 0.93 | 0.55 | 1.90 | 0.72 | 1.59 | 0.29 | 1.66 | 0.22 | | C76162 | | | | | | |
| Msa.27482.0 | 1.00 | −2.98 | −0.34 | 0.93 | −0.02 | 1.68 | 0.79 | 0.95 | 1.63 | 0.09 | DNA segment, | AA139094 | | | 10 41.7 | | | |
| | | | | | | | | | | | Chr 10, Johns | | | | cM | | | |
| | | | | | | | | | | | Hopkins | | | | | | | |
| | | | | | | | | | | | University 81 | | | | | | | |
| | | | | | | | | | | | expressed; | | | | | | | |
| | | | | | | | | | | | D10Jhu81e | | | | | | | |
| X65582 | 1.00 | −2.34 | −2.58 | 0.91 | −0.22 | 1.62 | 0.31 | 0.86 | 1.63 | 0.20 | procollagen, type | X65582 | collagen alpha | collagen vi acts as a | 10 41.1 | ECM | | |
| | | | | | | | | | | | VI, alpha 2; | | 2(vi) chain | cell-binding protein. | cM | (Matrix | | |
| | | | | | | | | | | | Col6a2 | | precursor. | | | Prot) | | |
| X58251 | 1.00 | −2.11 | −0.18 | 0.74 | 0.01 | 1.14 | 0.61 | 0.89 | 1.62 | 0.20 | procollagen, type | X58251 | collagen alpha | forms the fibrils of | 6 0.68 cM | ECM | | |
| | | | | | | | | | | | I, alpha 2; Cola2 | | 2(i) chain | tendon, ligaments and | | (Matrix | | |
| | | | | | | | | | | | | | precursor. | bones. in bones the | | Prot) | | |
| | | | | | | | | | | | | | | fibrils are mineralized | | | | |
| | | | | | | | | | | | | | | with calcium | | | | |
| | | | | | | | | | | | | | | hydroxyapatite | | | | |
| Msa.22727.0 | 1.00 | −2.67 | −0.62 | 2.27 | 0.42 | 1.67 | 1.45 | 1.31 | 1.61 | 0.93 | melanonia | AA038134 | | | | Structural | | |
| | | | | | | | | | | | X-actin; Actx | | | | | Protein | | |
| X66405 | 1.00 | −2 18 | −3.12 | 1.32 | −0.39 | 1.72 | −0.54 | 0.85 | 1.53 | 0.22 | procollagen, type | X66405 | collagen alpha | collagen vi acts as a | 10 41.1 | ECM | | |
| | | | | | | | | | | | VI, alpha 1; | | 1(vi) chain | cell-binding protein. | cM | (Matrix | | |
| | | | | | | | | | | | Col6a1 | | precursor. | | Prot) | | | |
| AF022256 | 1.00 | −3.92 | −1.46 | 0.47 | −0.36 | 1.65 | −1.65 | 1.42 | 1.49 | 0.18 | keratocan; Kera | AF022256 | | keratan sulfate | 10 61.0 | ECM | | |
| | | | | | | | | | | | | | | proteoglycans (kspgs) | cM | (Matrix | | |
| | | | | | | | | | | | | | | play a pivotal role in | | Prot) | | |
| | | | | | | | | | | | | | | the development and | | | | |
| | | | | | | | | | | | | | | maintenance of | | | | |
| | | | | | | | | | | | | | | corneal transparency. | | | | |
| | | | | | | | | | | | | | | keratocan, lumican, | | | | |
| | | | | | | | | | | | | | | and mimecan | | | | |
| | | | | | | | | | | | | | | (osteoglycin) are the | | | | |
| | | | | | | | | | | | | | | major keras in | | | | |
| Msa.22485.0 | 1.00 | −2.42 | −0.72 | 0.53 | 1.18 | 0.18 | 1.79 | 0.13 | 1.49 | 0.07 | | AA035834 | | | | | | |
| Msa.2851.0 | 1.00 | −2.52 | −4.32 | 2.71 | −0.35 | 1.82 | 1.71 | 0.13 | 1.46 | 0.14 | lipopolysaccha- | X99347 | lipopolysaccharide- | binds to the lipid a | 2 83.0 cM | | | |
| | | | | | | | | | | | ride binding | | binding protein | moiety of bacterial | | | | |
| | | | | | | | | | | | protein; Lbp | | precursor (lbp). | lipopolysaccharides | | | | |
| | | | | | | | | | | | | | (lps), a glycolipid | | | | | |
| | | | | | | | | | | | | | present in the outer | | | | | |
| | | | | | | | | | | | | | membrane of all gram- | | | | | |
| | | | | | | | | | | | | | negative bacteria the | | | | | |
| | | | | | | | | | | | | | lbp/lps complex seems | | | | | |
| | | | | | | | | | | | | | to interact with the | | | | | |
| | | | | | | | | | | | | | cd1d receptor | | | | | |
| Msa.117.0 | 1.00 | −2.41 | −1.85 | 0.94 | −0.06 | 1.16 | 1.28 | 0.10 | 1.42 | 0.08 | procollagen, type1, | U08020 | collagen alpha 1(i) | type i collagen is a | 11 56.0 cM | ECM | | |
| | | | | | | | | | | | alpha 1; Cola 1 | | chain precursor. | member of group i | | (Matrix | | |
| | | | | | | | | | | | | | collagen (fibrillar | | Prot) | | | |
| | | | | | | | | | | | | | forming collagen). | | | | | |
| Msa.3557.0 | 1.00 | −2 19 | −2.73 | 1.67 | −0.28 | 1.56 | −0.45 | 0.87 | 1.40 | 0.19 | stromal cell derived | W08269 | pigment epithelium- | neurotrophic protein; | | Cytokine | | |
| | | | | | | | | | | | factor 3; Sdf3 | | derived factor | induces extensive | | | | |
| | | | | | | | | | | | | precuraor (pedf) | neuronal | | | | | |
| | | | | | | | | | | | | (stromal cell- | differentiation in | | | | | |
| | | | | | | | | | | | | derived factor 3) | retinoblastoma cells. | | | | | |
| | | | | | | | | | | | | (sdf-3). | as it does not undergo | | | | | |
| | | | | | | | | | | | | | the s (stressed) to r | | | | | |
| | | | | | | | | | | | | | (relaxed) | | | | | |
| | | | | | | | | | | | | | conformational | | | | | |
| | | | | | | | | | | | | | transition | | | | | |
| | | | | | | | | | | | | | characteristic of active | | | | | |
| | | | | | | | | | | | | | serpins, it exhibits no | | | | | |
| | | | | | | | | | | | | | serine protease | | | | | |
| L29454 | 1.00 | −2.97 | −2.34 | 0.83 | −0.28 | 1.51 | 0.42 | 0.76 | 1.40 | 0.16 | fibrillin 1; Fbn1 | L29454 | fibrillin 1 precursor. | structural component | 2 71.0 cM | | | |
| | | | | | | | | | | | | | of connective tissue | | | | | |
| | | | | | | | | | | | | | microfibrils that binds | | | | | |
| | | | | | | | | | | | | | calcium. fibrillin-1- | | | | | |
| | | | | | | | | | | | | | containing microfibrils | | | | | |
| | | | | | | | | | | | | | provide long-term | | | | | |
| | | | | | | | | | | | | | force bearing | | | | | |
| | | | | | | | | | | | | | structural support | | | | | |
| AA689977 | 1.00 | −2.14 | −0.71 | 1.22 | 0.22 | 1.67 | 1.16 | 1.17 | 1.38 | 0.10 | mini chromosome | AA689977 | dna replication | may be involved in the | | | | |
| | | | | | | | | | | | maintenance | | licensing factor | control of a single | | | | |
| | | | | | | | | | | | deficient 6 (S. | | mcm6 (mis5 | round of dna | | | | |
| | | | | | | | | | | | cerevisiae); Mcmd6 | | homolog). | replication during a | | | | |
| | | | | | | | | | | | | | phasc. binds to | | | | | |
| | | | | | | | | | | | | | chromatin during g1 | | | | | |
| | | | | | | | | | | | | | and detach from it | | | | | |
| | | | | | | | | | | | | | during s phase as if it | | | | | |
| | | | | | | | | | | | | | licenses the chromatin | | | | | |
| | | | | | | | | | | | | | to replicate | | | | | |
| M18194 | 1.00 | −2.55 | −1.79 | 0.59 | 0.17 | 1.34 | 0.50 | 0.78 | 1.36 | 0.09 | fibronectin 1; Fn1 | M18194 | fibronectin | a glycoprotein that | 1 36.1 cM | Extracellular | | |
| | | | | | | | | | | | | precursor (fn) | interacts with a variety | | Protein | | | |
| | | | | | | | | | | | | (fragments) | of cells through both | | | | | |
| | | | | | | | | | | | | | integrin and non- | | | | | |
| | | | | | | | | | | | | | integrin receptors. | | | | | |
| | | | | | | | | | | | | | encoded by a single | | | | | |
| | | | | | | | | | | | | | gene, but alternative | | | | | |
| | | | | | | | | | | | | | splicing of pre-mma | | | | | |
| | | | | | | | | | | | | | allows formation of | | | | | |
| | | | | | | | | | | | | | multiple isoforms with | | | | | |
| | | | | | | | | | | | | | critical roles in cell | | | | | |
| | | | | | | | | | | | | | adhesion | | | | | |
| AA285530 | 1.00 | −2.38 | −0.40 | 0.60 | −1.07 | 0.02 | 0.66 | 0.94 | 1.35 | 0.14 | | AA285530 | | | | | | |
| Msa.10146.0 | 1.00 | 3 59 | −1.35 | 1.51 | 0.33 | 2.24 | 1.37 | 0.24 | 1.31 | 0.14 | vWF, human | AA168633 | | | | Hemostasis | | |
| X56602 | 1.00 | −7.23 | −2.12 | 0.76 | −5.94 | 0.81 | −1.36 | 0.13 | 1.25 | 1.56 | interferon-stimulated | X56602 | ubiquitin cross- | | | Cytokine | | |
| | | | | | | | | | | | protein (15 kDa); | | reactive protein | | | | | |
| | | | | | | | | | | | Isg15 | | (interferon- | | | | | |
| | | | | | | | | | | | | | stimulated protein | | | | | |
| | | | | | | | | | | | | 15). | | | | | | |
| Msa.419.0 | 1.00 | 16.53 | 1.33 | 1.41 | −1.51 | 0.36 | −0.88 | 1.00 | 1.09 | 1.75 | aminolevulinic | M63244 | 5-aminolevutinic | alas, though | X 63.0 cM | Intracellular | | |
| | | | | | | | | | | | acid synthase 2, | | acid synthase, | synthesized on | | Protein | | |
| | | | | | | | | | | | erythroid; | | erythroid-specific, | cytoplasmic | | | | |
| | | | | | | | | | | | Alas2 | | mitochondrial | ribosomes, | | | | |
| | | | | | | | | | | | | | precursor (ec | functions | | | | |
| | | | | | | | | | | | | | 2.3.1.37) (delta- | in | | | | |
| | | | | | | | | | | | | | aminolevulinate | mitochondria . . . | | | | |
| | | | | | | | | | | | | | synthase) (delta- | alas2 encodes | | | | |
| | | | | | | | | | | | | | ala synthetase) | the erythrocyte- | | | | |
| | | | | | | | | | | | | | (alas-e) | specific isoform | | | | |
| | | | | | | | | | | | | | | involved in | | | | |
| | | | | | | | | | | | | | | beme biosynthesis | | | | |
| U08020 | 1.00 | −3.63 | −2 35 | 1.03 | 0.52 | 2.00 | 1.42 | 0.36 | 1.07 | 0.81 | procollagen, type I, | U08020 | collagen alpha 1(i) | type i collagen is a | 11 56.0 cM | ECM | | |
| | | | | | | | | | | | alpha 1; Cola1 | | chain precursor. | member of group i | | (Matrix | | |
| | | | | | | | | | | | | | | colllagen (fibrillar | | Prot) | | |
| | | | | | | | | | | | | | | forming | | | | |
| | | | | | | | | | | | | | | collagen). | | | | |
| AA289002 | 1.00 | 2.49 | −0.24 | 0.54 | −1.32 | 0.24 | −0.42 | 1.25 | 1.03 | 0.69 | | AA289002 | | | | EST; | | |
| | | | | | | | | | | | | | | | | Unknown | | |
| AA684083 | 1.00 | −0.55 | 0.43 | 0.57 | −0.10 | 1.37 | 0.95 | 0.64 | 0.99 | 0.47 | | AA684083 | | | | | | |
| Msa 29141.0 | 1.00 | −2.49 | 0.35 | 0.77 | −0.06 | 1.40 | 0.94 | 1.18 | 0.96 | 0.67 | myosin Ic; Myolc | AA073795 | | | 11 44.13 | | | |
| | | | | | | | | | | | | | | | cM | | | |
| Msa 17336 0 | 1.00 | −2.35 | 0.72 | 0.79 | 0.14 | 1.33 | 0.88 | 1.00 | 0.94 | 0.70 | melanoma X-actin; | W89940 | | cells derived from the | | Structural | | |
| | | | | | | | | | | | Actx | | | murine b16 melanoma | | Protein | | |
| | | | | | | | | | | | | | | express a third actin | | | | |
| | | | | | | | | | | | | | | which has been | | | | |
| | | | | | | | | | | | | | | designated melanoma | | | | |
| | | | | | | | | | | | | | | x-actin. comparison | | | | |
| | | | | | | | | | | | | | | between x-actin and i_{—} | | | | |
| | | | | | | | | | | | | | | actin structures | | | | |
| | | | | | | | | | | | | | | indicate that x-actin is | | | | |
| | | | | | | | | | | | | | | inherited by a differnet | | | | |
| | | | | | | | | | | | | | | tissue actx | | | | |
| AA185262 | 1.00 | 2 41 | −0.69 | 0.73 | −0.01 | 1.41 | 1.73 | 1.87 | 0.89 | 0.79 | EST; Unknown | AA185262 | | | | EST; | | |
| | | | | | | | | | | | | | | | | Unknown | | |
| Msa.17592.0 | 1.00 | −2.77 | 0.35 | 0.83 | −0.30 | 1.33 | 1.52 | 0.24 | 0.86 | 0.65 | | W96831 | | | | | | |
| Msa.29918.0 | 1.00 | −2.55 | 0.41 | 1.16 | −0.02 | 1.09 | 1.29 | 1.17 | 0.85 | 0.64 | melanoma | AA087943 | | | | | | |
| | | | | | | | | | | | X-actin; | | | | | | | |
| | | | | | | | | | | | Actx | | | | | | | |
| AA396357 | 1.00 | 2.43 | −2.33 | 2.31 | 0.23 | 1.34 | 0.26 | 0.80 | 0.71 | 0.59 | ubiquitin-conjugating | AA396357 | | | 6 6.5 cM | | | |
| | | | | | | | | | | | enzyme E2H; Ube2h | | | | | | | |
| U77460 | 1.00 | 1.01 | −1.80 | 0.64 | −0.72 | 2.22 | 3.58 | 0.80 | 0.70 | 0.59 | complement | U77460 | c3a anaphylatoxin | aka: anaphylatoxin c3a | 6 1 1 | Hemostasis | | |
| | | | | | | | | | | | component 3a | | chemotactic | receptor, a g-protein | | | | |
| | | | | | | | | | | | receptor 1; C3ar1 | | receptor (c3a-r) | coupled receptor | | | | |
| | | | | | | | | | | | | | (c3ar) (complement | | | | | |
| | | | | | | | | | | | | | component 3a | | | | | |
| | | | | | | | | | | | | | receptor 1) | | | | | |
| Msa.4113.0 | 1.00 | 3.13 | −1.07 | 2.02 | 0.00 | 2.06 | −0.43 | 1.72 | 0.69 | 0.79 | glucocorticoid- | AA050733 | | expressed in normal | | Signal | | |
| | | | | | | | | | | | induced leucine | | | lymphocytes from | | Transduction | | |
| | | | | | | | | | | | zipper; Gilz | | | thymus, spleen, and | | | | |
| | | | | | | | | | | | | | | lymph nodes, low or | | | | |
| | | | | | | | | | | | | | | no expression detected | | | | |
| | | | | | | | | | | | | | | in other nonlymphoid | | | | |
| | | | | | | | | | | | | | | tissues, including | | | | |
| | | | | | | | | | | | | | | brain, kidney, and | | | | |
| | | | | | | | | | | | | | | liver, selectively | | | | |
| | | | | | | | | | | | | | | protects t cells from | | | | |
| | | | | | | | | | | | | | | apoptosis induced by | | | | |
| | | | | | | | | | | | | | | treatment with anti cd | | | | |
| AA222661 | 1.00 | −2.11 | −0.06 | 0.66 | −0.09 | 1.30 | −0.44 | 1.04 | 0.64 | 0.61 | | A222661 | | | | | | |
| AA530782 | 1.00 | 1.12 | −4.81 | 4.02 | −0.29 | 1 45 | −1.23 | 1.08 | 0.58 | 1.24 | keratin complex-1, | AA530782 | | | 11 57.85 | | | |
| | | | | | | | | | | | ene C29 Krt1-c29 | | | | cM | | | |
| AF033031 | 1.00 | −0.44 | 0.93 | 0.44 | 0.13 | 1.15 | 1.66 | 1.07 | 0.56 | 0.69 | solute carrier family | AF033031 | very-long-chain acyl | | | | | |
| | | | | | | | | | | | 27 (fatty acid | | coa synthetase (ec | | | | | |
| | | | | | | | | | | | transporter), member | | 6.2.1.—) (very-long- | | | | | |
| | | | | | | | | | | | 2; S1c27a2 | | chain-fatty-acid-coa | | | | | |
| | | | | | | | | | | | | | ligase). | | | | | |
| X03986 | 1.00 | 1.33 | −5.21 | 1.97 | −3.07 | 1.60 | 0.63 | 0.84 | 0.55 | 0.56 | acetylcholine | X03986 | acetylcholine | the alpha, beta, | 2 43.0 cM | Receptor | | |
| | | | | | | | | | | | receptor alpha; Acra | | receptor protein, | gamma, and delta | | | | |
| | | | | | | | | | | | | | alpha chain | subunits of the muscle | | | | |
| | | | | | | | | | | | | | precursor. | nicotinic acetylcholime | | | | |
| | | | | | | | | | | | | | | receptor, each | | | | |
| | | | | | | | | | | | | | | encoded by its own | | | | |
| | | | | | | | | | | | | | locus, are assembled | | | | | |
| | | | | | | | | | | | | | | into a pentamer of 2 | | | | |
| | | | | | | | | | | | | | | alpha units and one | | | | |
| | | | | | | | | | | | | | | each of the beta, | | | | |
| | | | | | | | | | | | | | | gamma and delta | | | | |
| AA611341 | 1.00 | 1.31 | −2.30 | 1.59 | −0.58 | 5.09 | −3.37 | 0.47 | 0.54 | 1.43 | keratin complex-1, | AA611341 | | | 11 57.85 | | | |
| | | | | | | | | | | | gene C29; Krt1-c29 | | | | cM | | | |
| AA240803 | 1.00 | 4.90 | 0.13 | 0.59 | −0.01 | 2.00 | 3.35 | 4.03 | 0.48 | 1.55 | Unknown, No hits | AA240803 | | | | EST; | | |
| | | | | | | | | | | | | | | | | Unknown | | |
| AB007848 | 1.00 | −2.59 | −1.24 | 0.53 | 0.22 | 1.88 | −0.61 | 1.14 | 0 39 | 0.87 | osteomodulin; Omd | AB007848 | | a novel bone matrix | | ECM | | |
| | | | | | | | | | | | | | | protein. | | (Matrix | | |
| | | | | | | | | | | | | | | | | Prot) | | |
| AA426892 | 1.00 | −2.91 | −2.01 | 0.99 | 0.18 | 2.47 | −0.02 | 1.21 | 0.28 | 1.23 | plasminogen | AA426892 | tissue-type | converts the abundant, | 8 9.0 cM | | | |
| | | | | | | | | | | | activator, tissue; | | plasminogen | but inactive, zymogen | | | | |
| | | | | | | | | | | | Plat | | activator precursor | plasminogen to | | | | |
| | | | | | | | | | | | | | (ec 3.4.21.68) (tpa) | plasmin by | | | | |
| | | | | | | | | | | | | | (t-pa) (t- | hydrolyzing a single | | | | |
| | | | | | | | | | | | | | | plasminogen | arg-val bond in | | | |
| | | | | | | | | | | | | | activator). | plasminogen. by | | | | |
| | | | | | | | | | | | | | | controlling plasmin- | | | | |
| | | | | | | | | | | | | | | mediated proteolysis, | | | | |
| | | | | | | | | | | | | | | it plays an important | | | | |
| | | | | | | | | | | | | | | role in tissue | | | | |
| | | | | | | | | | | | | | | remodeling and | | | | |
| | | | | | | | | | | | | | | degradation, in cell | | | | |
| | | | | | | | | | | | | | | migration and many | | | | |
| | | | | | | | | | | | | | | other | | | | |
| | | | | | | | | | | | | | | physiopathological | | | | |
| U16175 | 1.00 | −2.81 | −2.68 | 1.32 | −1.31 | 0.31 | −0.68 | 0.98 | 0.11 | 0.73 | hypothetical protein, | U16175 | mucin 1 precursor | a secreted | 3.3 42.6 | Extracellular | | |
| | | | | | | | | | | | mucin 1, | | (polymorphic | glycoprotein | cM.3 44.8 | Protein | | |
| | | | | | | | | | | | transmembrane, | | epithelial mucin) | member of the | cM | | | |
| | | | | | | | | | | | thrombospondin | | (pemt) | class of | | | | |
| | | | | | | | | | | | 3; | | (episialin)., | adhesive protein | | | | |
| | | | | | | | | | | | LOC54129, | | thrombo | have specialized | | | | |
| | | | | | | | | | | | MucI, Thbs3 | | spondin 3 | functions in cell | | | | |
| | | | | | | | | | | | | | precursor | growth, thbs3 | | | | |
| | | | | | | | | | | | | | | gene differs | | | | |
| | | | | | | | | | | | | | | markedly from | | | | |
| | | | | | | | | | | | | | | thbs1 and thbs2 | | | | |
| | | | | | | | | | | | | | | both in structure | | | | |
| | | | | | | | | | | | | | | expression | | | | |
| | | | | | | | | | | | | | | patterns | | | | |
| X14194 | 1.00 | −1.05 | −2.25 | 0.71 | −0.99 | 2.19 | −0.73 | 0.96 | 0.03 | 0.77 | nidogen 1; Nid1 | X14194 | | | 13 7.0 cM | | | |
| Msa.18310.0 | 1.00 | −2.23 | 0.10 | 1.07 | 0.05 | 1.53 | −0.59 | 0.93 | −0.05 | 0.74 | ATP citrate-lyase | AA000410 | | atp citrate-lyase is the | | Metabolic | | |
| | | | | | | | | | | | | | | primary enzyme | | | | |
| | | | | | | | | | | | | | | responsible for the | | | | |
| | | | | | | | | | | | | | | synthesis of cytosolic | | | | |
| | | | | | | | | | | | | | | acetyl-coa in many | | | | |
| | | | | | | | | | | | | | | tissues. strongly | | | | |
| | | | | | | | | | | | | | | expressed in liver and | | | | |
| | | | | | | | | | | | | | | adrenal, moderate | | | | |
| | | | | | | | | | | | | | | levels were found in | | | | |
| | | | | | | | | | | | | | | lung, brain, and large | | | | |
| | | | | | | | | | | | | | | intestine. of | | | | |
| | | | | | | | | | | | | | | importance in | | | | |
| Msa.18226.0 | 1.00 | −2.24 | −1.41 | 1.06 | −1.22 | 0.16 | −0.69 | 0.92 | −0.06 | 0.63 | CD34 antigen | AA000252 | | possible adhesion | | Cell Surface | | |
| | | | | | | | | | | | | | | molecule with a role | | Protein | | |
| | | | | | | | | | | | | | | in early hematopoiesis | | | | |
| | | | | | | | | | | | | | | by mediating the | | | | |
| | | | | | | | | | | | | | | attachment of stem | | | | |
| | | | | | | | | | | | | | | cells to the bone | | | | |
| | | | | | | | | | | | | | | marrow extracellular | | | | |
| | | | | | | | | | | | | | | matrix or directly to | | | | |
| | | | | | | | | | | | | | | stromal cells. could | | | | |
| | | | | | | | | | | | | | | act as a scaffold for | | | | |
| | | | | | | | | | | | | | | the attachment of | | | | |
| W40995 | 1.00 | 1.18 | −3.29 | 1.32 | −1.94 | 0.73 | −0.62 | 0.85 | −0.07 | 0.70 | | W40995 | | | | EST; | | |
| | | | | | | | | | | | | | | | | Unknown | | |
| U19118 | 1.00 | 2.01 | −0.96 | 0.81 | −0.83 | 1.97 | −1.82 | 0.18 | −0.09 | 0.66 | activating | U19118 | cyclic-amp- | this protein binds the | | | | |
| | | | | | | | | | | | tranacription factor | | dependent | camp response | | | | |
| | | | | | | | | | | | 3; Atf3 | | transcription factor | element (cre) | | | | |
| | | | | | | | | | | | | | atf-3 (activating | (consensus: | | | | |
| | | | | | | | | | | | | | transcription factor | 5′gtgacgt(a/c)(a/g)-3′), | | | | |
| | | | | | | | | | | | | | 3) (transcription | a sequence present ing | | | | |
| | | | | | | | | | | | | | factor 1rg-21). | many viral and | | | | |
| | | | | | | | | | | | | | | cellular promotors. | | | | |
| | | | | | | | | | | | | | | represses transcription | | | | |
| | | | | | | | | | | | | | | from promotors with | | | | |
| | | | | | | | | | | | | | | arf sites. it may repress | | | | |
| | | | | | | | | | | | | | | transcription by | | | | |
| | | | | | | | | | | | | | | stabilizing the binding | | | | |
| | | | | | | | | | | | | | | of inhibitory co- | | | | |
| Msa.6450.0 | 1.00 | −2.94 | −2.22 | 1.18 | −1.31 | 0.28 | −1.43 | 0.25 | −0.13 | 0.72 | early quiescence | AA038318 | | | | | | |
| | | | | | | | | | | | protein-1; Eg1 | | | | | | | |
| AA028265 | 1.00 | −2.59 | −1.71 | 1.05 | 0.65 | 2.25 | −0.62 | 1.07 | −0.13 | 1.09 | fibromodulin | AA028265 | | fibromodulin, small | | ECM | | |
| | | | | | | | | | | | | | | collagen-binding | | (Matrix | | |
| | | | | | | | | | | | | | | proteoglycan of the | | Prot) | | |
| | | | | | | | | | | | | | | extra-cellular matrix, | | | | |
| | | | | | | | | | | | | | | mainly expressed in | | | | |
| | | | | | | | | | | | | | | articular cartilage, | | | | |
| | | | | | | | | | | | | | | tendon, ligament, | | | | |
| | | | | | | | | | | | | | | leucine-rich repeat | | | | |
| | | | | | | | | | | | | | | (1rr) family believed to | | | | |
| | | | | | | | | | | | | | | function in the | | | | |
| | | | | | | | | | | | | | | assembly of the | | | | |
| | | | | | | | | | | | | | | collagen network in | | | | |
| Msa.15338.0 | 1.00 | 2.11 | −0.56 | 1.01 | −0.01 | 1.28 | −0.56 | 1.01 | −0.13 | 0.74 | glucocorticoid- | AA097366 | | expressed in normal | | Transcription | | |
| | | | | | | | | | | | induced leucine | | | lymphocytes from | | Factor | | |
| | | | | | | | | | | | zipper; Gilz | | | thymus, spleen, and | | | | |
| | | | | | | | | | | | | | | lymph nodes, low or | | | | |
| | | | | | | | | | | | | | | no expression detected | | | | |
| | | | | | | | | | | | | | | in other nonlymphoid | | | | |
| | | | | | | | | | | | | | | tissues, including | | | | |
| | | | | | | | | | | | | | | brain, kidney, and | | | | |
| | | | | | | | | | | | | | | liver. selectively | | | | |
| | | | | | | | | | | | | | | protects t cells from | | | | |
| | | | | | | | | | | | | | | apoptosis induced by | | | | |
| | | | | | | | | | | | | | | treatment with anti ad | | | | |
| Msa.22717.0 | 1.00 | 1.99 | −2.86 | 2.12 | −0.02 | 1.43 | −1.17 | 1.15 | −0.17 | 0.99 | actinin alpha3; | AA038082 | | | | | | |
| | | | | | | | | | | | Actn3 | | | | | | | |
| M22326-2 | 1.00 | −1.55 | −1.30 | 0.87 | −2.26 | 0.11 | −1.73 | 0.45 | −0.29 | 0.85 | early growth | M22326 | early growth | transcriptional | 18 16.0 cM | Intracellular | | |
| | | | | | | | | | | | response 1; | | response protein 1 | regulator. recognizes | | Protein | | |
| | | | | | | | | | | | EgrI | | (egr-1) (krox-24 | and binds to the dna | | | | |
| | | | | | | | | | | | | | protein) (zif268). | sequence 5′-cgcccccgc- | | | | |
| | | | | | | | | | | | | | | 3′(egr-site). activates | | | | |
| | | | | | | | | | | | | | | the transcription of | | | | |
| | | | | | | | | | | | | | | target genes whose | | | | |
| | | | | | | | | | | | | | | products are required | | | | |
| | | | | | | | | | | | | | | for mitogenesis and | | | | |
| | | | | | | | | | | | | | | differentiation | | | | |
| AA028499 | 1.00 | 1.67 | −2.88 | 0.88 | −0.48 | 1.69 | −2.20 | 0.37 | −0.32 | 1.28 | | AA028499 | | | | | | |
| Msa.5528.0 | 1.00 | −2.83 | −3.31 | 0.38 | −3.53 | 2.03 | −2.06 | 0.49 | −0.44 | 0.97 | retinol binding | W11638 | plasma retinol- | rbp delivers retinol | 19 38.0 cM | | | |
| | | | | | | | | | | | protein 4, plasma; | | binding protein | from the liver stores to | | | | |
| | | | | | | | | | | | Rbp4 | | precursor (prbp) | the peripheral tissues. | | | | |
| | | | | | | | | | | | | | (rbp). | in plasma, the rbp- | | | | |
| | | | | | | | | | | | | | | retinol complex | | | | |
| | | | | | | | | | | | | | | interacts with | | | | |
| | | | | | | | | | | | | | | transthyretin, this | | | | |
| | | | | | | | | | | | | | | prevents its loss by | | | | |
| | | | | | | | | | | | | | | filtration through the | | | | |
| | | | | | | | | | | | | | | kidney glomeruli | | | | |
| W35693 | 1.00 | 2.57 | −1.92 | 0.93 | −0.25 | 1.56 | −0.30 | 0.98 | −0.61 | 0.56 | EST; Unknown | W35693 | | | | EST; | | |
| | | | | | | | | | | | | | | | | Unknown | | |
| Msa.2579.0 | 1.00 | −2.09 | −3.10 | 1.53 | −0.55 | 1.69 | −2.70 | 0.93 | −0.63 | 0.78 | DNA segment, | X70398 | | expressed in cns | | Other | | |
| | | | | | | | | | | | human D4S114; | | | | | | | |
| AA048018 | 1.00 | 2.01 | −1.54 | 0.61 | 0.10 | 1.39 | −0.52 | 0.89 | −0.70 | 0.69 | | AA048018 | | | | | | |
| Msa.2975.0 | 1.00 | −2.13 | −3.13 | 1.51 | −0.57 | 1.76 | −0.61 | 0.84 | −0.71 | 0.66 | retinol binding | W14367 | | plasma retinol binding | | Extracellular | | |
| | | | | | | | | | | | protein 4, plasma | | | protein (rbp4) and | | Protein | | |
| | | | | | | | | | | | | | | transthyretin complex | | | | |
| | | | | | | | | | | | | | | with retinol to | | | | |
| | | | | | | | | | | | | | | transport it from | | | | |
| | | | | | | | | | | | | | | storage sites in the | | | | |
| | | | | | | | | | | | | | | liver to target tissues. | | | | |
| | | | | | | | | | | | | | | retinol modulates | | | | |
| | | | | | | | | | | | | | | epithelial | | | | |
| | | | | | | | | | | | | | | morphogenesis and | | | | |
| | | | | | | | | | | | | | | epithelial | | | | |
| | | | | | | | | | | | | | | differentiation | | | | |
| X59060 | 1.00 | 1.85 | −0.94 | 0.71 | −0.32 | 1.40 | 4.80 | 1.20 | −0.72 | 0.81 | myogenic factor 6; | X59060 | myogenic factor | myf6 or herculin is | 10 59.0 cM | Trancription | | |
| | | | | | | | | | | | Myf6 | | myf-6 (herculin). | expressed in adult | | Factor | | |
| | | | | | | | | | | | | | | skeletal muscle, but | | | | |
| | | | | | | | | | | | | | | not in smooth muscle, | | | | |
| | | | | | | | | | | | | | | cardiac muscle, or non- | | | | |
| | | | | | | | | | | | | | | muscular tissues it | | | | |
| | | | | | | | | | | | | | | activates expression of | | | | |
| | | | | | | | | | | | | | | myod1 and myog. the | | | | |
| | | | | | | | | | | | | | | level of expression of | | | | |
| | | | | | | | | | | | | | | herculin is higher than | | | | |
| | | | | | | | | | | | | | | for any of the other | | | | |
| Msa.15200.0 | 1.00 | −3.48 | −3.48 | 2.00 | −1.38 | 0.16 | −1.75 | 0.30 | −0.72 | 0.58 | CD34 antigen | W65699 | | possible adhesion | | Cell Surface | | |
| | | | | | | | | | | | | | | molecule with a role | | Protein | | |
| | | | | | | | | | | | | | | in early hematopoiesis | | | | |
| | | | | | | | | | | | | | | by mediating the | | | | |
| | | | | | | | | | | | | | | attachment of stem | | | | |
| | | | | | | | | | | | | | | cells to the bone | | | | |
| | | | | | | | | | | | | | | marrow extracellular | | | | |
| | | | | | | | | | | | | | | matrix or directly to | | | | |
| | | | | | | | | | | | | | | stromal cells. could | | | | |
| | | | | | | | | | | | | | | act as a scaffold for | | | | |
| | | | | | | | | | | | | | | the attachment of | | | | |
| AA530179 | 1.00 | 1.05 | −3.59 | 2.01 | −0.08 | 2.05 | −3.74 | 0.67 | −0.74 | 1.69 | S100 calcium | AA530179 | s100 calcium- | binds both | 3 43.6 cM | | | |
| | | | | | | | | | | | binding protein | | binding protein a3 | calcium and | | | | |
| | | | | | | | | | | | | | A3;S100a3 | | (s-100e protein). | zinc. probably | | |
| | | | | | | | | | | | | | | binds 2 zinc ions | | | | |
| | | | | | | | | | | | | | | per molecule (by | | | | |
| Msa.7352.0 | 1.00 | 1.81 | −2.64 | 0.53 | −0.82 | 2.09 | −0.65 | 0.85 | −0.76 | 0.70 | | AA008667 | | | | | | |
| AA562685 | 1.00 | −1.16 | −2.39 | 0.61 | −0.55 | 2.04 | −0.95 | 1.17 | −0.79 | 0.84 | procollagen, type I, | AA562685 | | type i collagen is of | | ECM | | |
| | | | | | | | | | | | alpha 1 | | | particular importance | | (Matrix | | |
| | | | | | | | | | | | | | | in the extracellular | | Prot) | | |
| | | | | | | | | | | | | | | matrix of bone, skin, | | | | |
| | | | | | | | | | | | | | | tendon, and dentine, | | | | |
| | | | | | | | | | | | | | | and is highly | | | | |
| | | | | | | | | | | | | | | expressed in | | | | |
| | | | | | | | | | | | | | | fibroblasts. it is known | | | | |
| | | | | | | | | | | | | | | to be expressed in | | | | |
| | | | | | | | | | | | | | | mouse palatal shelves | | | | |
| | | | | | | | | | | | | | | during development“” | | | | |
| Maa.723.0 | 1.00 | −2.06 | −2.76 | 1.29 | −1.31 | 0.12 | −0.57 | 0.81 | −0.82 | 0.62 | aquaporin 1; Aqp1 | L02914 | aquaporin-chip | forms a water-specific | 6 27.0 cM | Cell Surface | | |
| | | | | | | | | | | | | | (water channel | channel that provides | | Protein | | |
| | | | | | | | | | | | | | protein for red | the plasma membranes | | | | |
| | | | | | | | | | | | | | blood cells and | of red cells and kidney | | | | |
| | | | | | | | | | | | | | kidney proximal | proximal tubules with | | | | |
| | | | | | | | | | | | | | tubule) (aquaporin | high permeability to | | | | |
| | | | | | | | | | | | | | 1) (early response | water | | | | |
| | | | | | | | | | | | | | protein der2) | | | | | |
| Msa.17890.0 | 1.00 | 1.98 | −3.38 | 2.16 | −0.23 | 1.33 | −1.21 | 0.05 | −0.86 | 0.69 | cukaryotic | W98531 | | | | | | |
| | | | | | | | | | | | translation | | | | | | | |
| | | | | | | | | | | | elongation factor | | | | | | | |
| | | | | | | | | | | | 2 Eef2 | | | | | | | |
| K02108 | 1.00 | 2.14 | −0.92 | 1.29 | 0.82 | 1.99 | 0.19 | 1.31 | −0.86 | 0.84 | keratin complex 2, | K02108 | keratin, type ii | there are two types of | 15 | | | |
| | | | | | | | | | | | gene 6a; Krt2-6a | | cytoskeletal 6 | cytoskeletal and | | | | |
| | | | | | | | | | | | | | (cytokeratin 6) (ck | microfibrillar keratin: | | | | |
| | | | | | | | | | | | | | 6) (k6 keratin) | i (acidic; 4-55 kda) | | | | |
| | | | | | | | | | | | | | | [k9 to k2] and ii | | | | |
| | | | | | | | | | | | | | | (neutral to basic; 56-7 | | | | |
| | | | | | | | | | | | | | | kda) [k1 to k8]. both a | | | | |
| | | | | | | | | | | | | | | basic and an acidic | | | | |
| | | | | | | | | | | | | | | keratin are required | | | | |
| | | | | | | | | | | | | | | for filament assembly | | | | |
| AA185284 | 1.00 | 2 23 | −2.22 | 0.43 | −0.83 | 1.88 | −1.32 | 0.14 | −0.86 | 0.64 | | AA185284 | | | | | | |
| W41417 | 1.00 | 3.44 | −2.19 | 1.11 | −0.36 | 1.43 | −1.53 | 0.23 | −0.94 | 0.83 | | W41417 | | | | | | |
| Msa.1170.0 | 1.00 | 1.37 | −3.82 | 2.50 | 0.06 | 2.16 | −2.69 | 0.44 | −0.98 | 1.39 | kerstin-associated | M37759 | | keratin-associated | | Other | | |
| | | | | | | | | | | | protein 5-1; Krtap5-1 | | | | | | | |
| W81858 | 1.00 | 1.23 | −2.53 | 0.85 | −0.68 | 1.89 | −1.81 | 1.47 | −1.07 | 0.94 | kinesin light chain 1; | W81858 | | | 12 57.0 cM | | | |
| | | | | | | | | | | | K1c1 | | | | | | | |
| C77823 | 1.00 | 1.13 | −1.83 | 0.58 | −0.57 | 2.16 | −0.67 | 1.04 | −1.08 | 0.83 | | C77823 | | | | | | |
| AF020194 | 1.00 | 1.05 | −3.83 | 2.27 | −0.41 | 1.77 | −1.05 | 1.19 | −1.08 | 0.80 | taurine/beta-alanine | AF020194 | sodium- and | an amino acid | 6 38.2 cM | Cell Surface | | |
| | | | | | | | | | | | transporter; Taut | | chloride-dependent | transporter, found | | Protein | | |
| | | | | | | | | | | | | | taurine and beta- | primarily in brain. | | | | |
| | | | | | | | | | | | | | alanine transporter. | | | | | |
| Msa.43191.0 | 1.00 | 1.18 | −3.36 | 0.99 | −1.28 | 2.65 | −0.86 | 1.24 | −1.09 | 0.96 | integrin-associated | Z25524 | | | | Cell Surface | | |
| | | | | | | | | | | | protein; Itgp | | | | | | | |
| X63023 | 1.00 | 2.54 | 0.34 | 0.51 | 0.25 | 1.30 | −1.13 | 0.04 | −1.14 | 0.03 | cytochrome P450, | X63023 | cytochrome p450 | can activate aflatoxin | 5 | | | |
| | | | | | | | | | | | steroid inducible | | 3a13 (ec 1.14.14.1) | b1 to a genotoxic | | | | |
| | | | | | | | | | | | 3a13; Cypiiia13). | product. | | | | | | |
| Msa.22263.0 | 1.00 | −2.02 | −1.25 | 1.32 | −0.12 | 1.28 | −0.50 | 0.92 | −1.17 | 0.11 | | AA033333 | | | | | | |
| C80656 | 1.00 | 1.58 | −3.32 | 1.44 | −0.59 | 1.91 | −0.79 | 2.01 | −1.20 | 0.87 | Unknown | C80656 | | | | EST; | | |
| | | | | | | | | | | | | | | | | Unknown | | |
| Msa.1531.0 | 1.00 | 1.97 | −2.54 | 1.23 | −1.29 | 0.24 | −0.65 | 0.88 | −1.21 | 0.07 | apolipoprotein D; | L39123 | apolipoprotein d | apod occurs in the | 16 21.2 cM | Extracellular | | |
| | | | | | | | | | | | Apod | | precursor. | macromolecular | | Protein | | |
| | | | | | | | | | | | | | | complex with | | | | |
| | | | | | | | | | | | | | | lecithin- | | | | |
| | | | | | | | | | | | | | | cholesterol | | | | |
| | | | | | | | | | | | | | | acyltransferase. | | | | |
| | | | | | | | | | | | | | | it is probably | | | | |
| | | | | | | | | | | | | | | involved in | | | | |
| | | | | | | | | | | | | | | the transport and | | | | |
| | | | | | | | | | | | | | | binding of bilin. | | | | |
| | | | | | | | | | | | | | | appears to be able | | | | |
| | | | | | | | | | | | | | | to transport a | | | | |
| | | | | | | | | | | | | | | variety of ligands | | | | |
| | | | | | | | | | | | | | | in a number of | | | | |
| | | | | | | | | | | | | | | different contexts | | | | |
| Msa.9372.0 | 1.00 | 1.75 | −2.72 | 1.03 | −1.67 | 0.58 | −2.02 | 0.43 | −1.22 | 0.76 | CD59 antigen; Cd59 | W41339 | | | 2 55.0 cM | | | |
| Msa.383.0 | 1.00 | 2.36 | −1.59 | 2.09 | −0.22 | 1.48 | −1.20 | 0.07 | −1.28 | 0.12 | erythrocyte protein | L00919 | protein 4.1 (band | protein 4.1 is a major | 4 65.7 cM | Intracellular | | |
| | | | | | | | | | | | band4.1; Epb4.1 | | 4.1) (p4.1). | structural element of | | Protein | | |
| | | | | | | | | | | | | | | the erythrocyte | | | | |
| | | | | | | | | | | | | | | membrane | | | | |
| | | | | | | | | | | | | | | skeleton. it | | | | |
| | | | | | | | | | | | | | | plays a key role | | | | |
| | | | | | | | | | | | | | | in regulating | | | | |
| | | | | | | | | | | | | | | membrane | | | | |
| | | | | | | | | | | | | | | physical | | | | |
| | | | | | | | | | | | | | | properties of | | | | |
| | | | | | | | | | | | | | | mechanical | | | | |
| | | | | | | | | | | | | | | stability | | | | |
| | | | | | | | | | | | | | | and deformability | | | | |
| | | | | | | | | | | | | | | by stabilizing | | | | |
| | | | | | | | | | | | | | | spectrin-actin | | | | |
| | | | | | | | | | | | | | | interaction. binds | | | | |
| | | | | | | | | | | | | | | with a high | | | | |
| | | | | | | | | | | | | | | affinity to | | | | |
| | | | | | | | | | | | | | | Mglycophorin | | | | |
| | | | | | | | | | | | | | | and with | | | | |
| | | | | | | | | | | | | | | lower affinity | | | | |
| | | | | | | | | | | | | | | to band | | | | |
| X82648 | 1.00 | 2.15 | −1.74 | 0.84 | −1.17 | 0.14 | −0.69 | 0.88 | −1.32 | 0.08 | apolipoprotein D; | X82648 | apolipoprotein d | apod occurs in the | 16 21.2 cM | Other | | |
| | | | | | | | | | | | Apod | | precursor. | macromolecular | | | | |
| | | | | | | | | | | | | | | complex with lecithin- | | | | |
| | | | | | | | | | | | | | | transport and binding | | | | |
| | | | | | | | | | | | | | | of bilin. appears to be | | | | |
| | | | | | | | | | | | | | | able to transport a | | | | |
| | | | | | | | | | | | | | | variety of ligands in a | | | | |
| | | | | | | | | | | | | | | number of different | | | | |
| | | | | | | | | | | | | | | contexts | | | | |
| Msa.5789 0 | 1.00 | 1.98 | −3.40 | 2.07 | −0.23 | 1.46 | −0.65 | 1.07 | −1.34 | 0.16 | | W18503 | | | | | | |
| AA673431 | 1.00 | −2.43 | −0.84 | 0.75 | 0.23 | 1.52 | 0.71 | 1.09 | −1.35 | 0.19 | | AA673431 | | | | EST; | | |
| | | | | | | | | | | | | | | | | Unknown | | |
| Msa.5254.0 | 1.00 | −2.55 | −2.67 | 1.27 | −1.48 | 0.08 | −1.88 | 0.35 | −1.43 | 0.13 | | AA064307 | | | | | | |
| Msa.21971.0 | 1.00 | 1.10 | −4.46 | 1.91 | −1.55 | 2.64 | −0.54 | 1.03 | −1.44 | 0.12 | | AA154451 | | | | | | |
| Msa.14179.0 | 1.00 | 2.98 | −2.90 | 1.25 | −0.15 | 1.67 | −0.92 | 1.23 | −1.47 | 0.96 | solute carrier | AA118682 | | | | | | |
| | | | | | | | | | | | family 25 | | | | | | | |
| | | | | | | | | | | | (mitochondrial | | | | | | | |
| | | | | | | | | | | | carrier; | | | | | | | |
| | | | | | | | | | | | peroxisomal | | | | | | | |
| | | | | | | | | | | | membrane | | | | | | | |
| | | | | | | | | | | | protein 34 kDa), | | | | | | | |
| | | | | | | | | | | | member 17; | | | | | | | |
| | | | | | | | | | | | Slc25a17 | | | | | | | |
| AA162560 | 1.00 | 1.07 | −2.54 | 0.34 | −2.59 | 0.16 | −1.60 | 0.16 | −1.47 | 0.15 | | AA162560 | | | | | | |
| X57024 | 1.00 | 1.10 | −4.45 | 2.19 | −1.76 | 0.36 | −1.55 | 0.25 | −1.48 | 0.18 | glutamate | X57024 | glutamate | | 14 15.5 cM | Metabolic | | |
| | | | | | | | | | | | dehydrogenase; | | dehydrogenase | | | | | |
| | | | | | | | | | | | Glud | | precursor (ec | | | | | |
| | | | | | | | | | | | | | 1.4.1.3) (gdh). | | | | | |
| AA198316 | 1.00 | 2.64 | −0.11 | 0.83 | 1.38 | 0.08 | 0.33 | 1.10 | −1.51 | 0.18 | acyl-CoA thioesterase | AA198316 | | | 12 | | | |
| | | | | | | | | | | | 1, cytosolic; Cte1- | | | | | | | |
| | | | | | | | | | | | pending | | | | | | | |
| Msa.2414.0 | 1.00 | 1.72 | −2.35 | 1.26 | −0.70 | 0.64 | −1.30 | 0.17 | −1.53 | 0.19 | laminin, | X84014 | laminin alpha-3 | laminin-5 is | 18 3.0 cM | | | |
| | | | | | | | | | | | alpha 3; Lama3 | | chain precursor | thought to be | involved in (1) | | | |
| | | | | | | | | | | | | | (fragment). | involved in (1) | | | | |
| | | | | | | | | | | | | | | cell adhesion | | | | |
| | | | | | | | | | | | | | | via integrin | | | | |
| | | | | | | | | | | | | | | alpha-3/beta- | | | | |
| | | | | | | | | | | | | | | 1 in focal | | | | |
| | | | | | | | | | | | | | | adhesion and | | | | |
| | | | | | | | | | | | | | | integrin | | | | |
| | | | | | | | | | | | | | | alpha-6/beta-4 in | | | | |
| | | | | | | | | | | | | | | hemidesmosomes. | | | | |
| | | | | | | | | | | | | | | (2) signal | | | | |
| | | | | | | | | | | | | | | transduction via | | | | |
| | | | | | | | | | | | | | | tyrosine | | | | |
| | | | | | | | | | | | | | | phosphorylation | | | | |
| | | | | | | | | | | | | | | of pp125-fak and | | | | |
| | | | | | | | | | | | | | | p8, (3) | | | | |
| | | | | | | | | | | | | | | differentiation of | | | | |
| | | | | | | | | | | | | | | keratinocytes (by | | | | |
| AA237919 | 1.00 | −0.03 | −2.41 | 0.90 | −0.29 | 1.68 | −1.58 | 0.35 | −1.53 | 0.46 | | AA237919 | | | | | | |
| AA028657 | 1.00 | 1.12 | −2.28 | 0.70 | −0.54 | 1.87 | −2.01 | 0.46 | −1.60 | 1.03 | EST; Unknown | AA028657 | | | | EST; | | |
| | | | | | | | | | | | | | | | | Unknown | | |
| Msa.330.0 | 1.00 | 1.53 | −5.88 | 2.92 | −2.03 | 0.91 | −1.61 | 0 39 | −1.63 | 0.36 | upstream | U12283 | upstream | bb1h protein that is | 7 11.0 cM | Transcription | | |
| | | | | | | | | | | | transcription | | stimulatory factor | ubiquitously | | Factor | | |
| | | | | | | | | | | | factor 2; Usf2 | | 2 (upstream | expressed. | | | | |
| | | | | | | | | | | | | | transcription factor | transcription factor | | | | |
| | | | | | | | | | | | | | 2) (major late | that binds to e-boxes | | | | |
| | | | | | | | | | | | | | transcription factor | (5′-cacgtg-3′) found in | | | | |
| | | | | | | | | | | | | | 2). | a variety of viral and | | | | |
| | | | | | | | | | | | | | | cellular promotors. | | | | |
| | | | | | | | | | | | | | | forms bb1h dimers for | | | | |
| | | | | | | | | | | | | | | dna binding. binds dna | | | | |
| | | | | | | | | | | | | | | as homodimer or | | | | |
| | | | | | | | | | | | | | | heterodimer | | | | |
| Msa.40899.0 | 1.00 | 1.40 | −2.51 | 0.86 | −1.22 | 0.17 | −3.58 | 0.96 | −1.64 | 1.30 | EST to MYH8 | AA162395 | | m36769 homo sapiens | | | | |
| | | | | | | | | | | | | | | permatal myosin | | | | |
| | | | | | | | | | | | | | | heavy chain 8% to | | | | |
| | | | | | | | | | | | | | | human | | | | |
| Msa.42549.0 | 1.00 | −2.37 | −0.31 | 1.04 | −0.14 | 1.77 | −1.06 | 1.30 | −1.65 | 0.27 | est | AA168690 | | similar to calmodulin | | EST; | | |
| | | | | | | | | | | | | | | | | Unknown | | |
| AA266377 | 1.00 | 2.21 | −1.86 | 0.25 | −1.53 | 0.40 | −1.67 | 0.31 | −1.69 | 0.22 | | AA266377 | | | | | | |
| Msa 8112.0 | 1.00 | −2.17 | −2.20 | 0.84 | −0.38 | 1.57 | −0.54 | 0.89 | −1.71 | 0.97 | CD151 antigen; | AA050218 | platelet-endothelial | | 7 23.5 cM | | | |
| | | | | | | | | | | | Cd151 | | tetraspan antigen 3 | | | | | |
| | | | | | | | | | | | | | (peta−3) (gp27) | | | | | |
| | | | | | | | | | | | | | (membrane | | | | | |
| | | | | | | | | | | | | | glycoprotein sfa-1) | | | | | |
| | | | | | | | | | | | | | (cd151 antigen) | | | | | |
| D17577 | 1.00 | −2.15 | −0.33 | 0.69 | 1.25 | 0.06 | −1.82 | 0.43 | −1.72 | 0.24 | kinesin heavy | D17577 | kinesin-like | kif1b works as a | 4 70.9 cM | Structural | | |
| | | | | | | | | | | | chain member | | protein kif1b. | monomeric motor | | Protein | | |
| | | | | | | | | | | | 1B; Kif1b | | for anterograde | | | | | |
| | | | | | | | | | | | | | | transport of | | | | |
| | | | | | | | | | | | | | | mitochondria | | | | |
| Msa.2160.0 | 1.00 | 1.96 | −6.25 | 5.84 | −0.10 | 1.23 | −1.32 | 0.08 | −1.72 | 0.13 | apolipoprotein CI; | AA049273 | apolipoprotein c-i | low molecular | 7 4.0 cM | Other | | |
| | | | | | | | | | | | Apoc1 | | precursor | surface | | | | |
| | | | | | | | | | | | | | (apo-ci) | weight surface | | | | |
| | | | | | | | | | | | | | component of | | | | | |
| | | | | | | | | | | | | | chylomicrons and | | | | | |
| | | | | | | | | | | | | | of very low | | | | | |
| | | | | | | | | | | | | | density (v1d1) | | | | | |
| | | | | | | | | | | | | | and high density | | | | | |
| | | | | | | | | | | | | | (hd1) | | | | | |
| | | | | | | | | | | | | | lipoproteins. | | | | | |
| | | | | | | | | | | | | | functions may | | | | | |
| | | | | | | | | | | | | | include activation | | | | | |
| | | | | | | | | | | | | | of lecithin: | | | | | |
| | | | | | | | | | | | | | cholesterol | | | | | |
| | | | | | | | | | | | | | acyltransferase, | | | | | |
| | | | | | | | | | | | | | and inhibition | | | | | |
| | | | | | | | | | | | | | of apoe binding | | | | | |
| | | | | | | | | | | | | | to the 1d1 rac | | | | | |
| U37222 | 1.00 | −2.07 | −1.65 | 0.64 | −1.51 | 0.04 | −3.13 | 1.34 | −1.73 | 0.14 | adipocyte | U37222 | 30 kda adipocyte | may function as a | | | | |
| | | | | | | | | | | | complement related | | complement-related | signaling molecule for | | | | |
| | | | | | | | | | | | protein of 30 kDa; | | protein precursor | adipose tissue. | | | | |
| | | | | | | | | | | | Acrp30 | | (acrp30) (adipocyte | | | | | |
| | | | | | | | | | | | | | specific protein | | | | | |
| | | | | | | | | | | | | | adipog). | | | | | |
| AA237797 | 1.00 | 1.15 | −2.54 | 0.76 | −0.57 | 1.88 | −2.14 | 0.54 | −1.75 | 1.03 | EST; Unknown | AA237797 | | | | EST; | | |
| | | | | | | | | | | | | | | | | Unknown | | |
| Msa.33047.0 | 1.00 | −2.09 | −1.89 | 0.98 | 0.71 | 0.91 | 1.19 | 0.14 | −1.76 | 1.51 | CD151 antigen; | AA109912 | platelet- | gene expression was | 7 23.5 cM | Cell Surface | | |
| | | | | | | | | | | | Cd151 | | endothelial | was observed in | | Protein | | |
| | | | | | | | | | | | | | tetraspan antigen | many cell types | | | | |
| | | | | | | | | | | | | | 3 (peta-3)(gp27) | but was either | | | | |
| | | | | | | | | | | | | | (membrane | absent or present | | | | |
| | | | | | | | | | | | | | glycoprotein | at a low level | | | | |
| | | | | | | | | | | | | | sfa-1) | in brain and | | | | |
| | | | | | | | | | | | | | (cd151 antigen). | lymphoid cells | | | | |
| | | | | | | | | | | | | | | tissues, including | and tissues, | | | |
| | | | | | | | | | | | | | | thymus and spleen. | including thymus | | | |
| | | | | | | | | | | | | | | contains four putative | and spleen | | | |
| | | | | | | | | | | | | | | transmembrane | contains four | | | |
| | | | | | | | | | | | | | | domains, a number of | putative | | | |
| | | | | | | | | | | | | | | cysteine residues | transmembrane | | | |
| | | | | | | | | | | | | | | domains, a | | | | |
| | | | | | | | | | | | | | | number of | | | | |
| Msa.370.0 | 1.00 | 1.28 | −2.88 | 0.74 | −1.69 | 0.28 | −1.62 | 0.39 | −1.79 | 0.35 | peroxisomal | L27842 | peroxisome | somewhat | | | | |
| | | | | | | | | | | | membrane | | assembly factor-1 | implicates in the | | | | |
| | | | | | | | | | | | protein 3, | | (paf-1) | biogenesis of | | | | |
| | | | | | | | | | | | | | (peroxin-2) | peroxisomes. | | | | |
| Msa.13629.0 | 1.00 | 1.77 | −5.94 | 4.06 | −1.56 | 0.40 | −1.67 | 0.26 | −1.80 | 0.36 | | AA155371 | | | | | | |
| Msa.43204.0 | 1.00 | 1.11 | −1.96 | 0.42 | −1.63 | 0.42 | −2.18 | 0.61 | −1.85 | 0.83 | serine protease | M75721 | alpha-1- | inhibitor of | 12 51.0 cM | | | |
| | | | | | | | | | | | inhibitor 1-1; | | antitrypsin | serine proteases. | | | | |
| | | | | | | | | | | | Spil-1 | | 1-1 precursor | its primary | | | | |
| | | | | | | | | | | | | | (serine protease | target is | | | | |
| | | | | | | | | | | | | | inhibitor 1-1) | elastase, but it | | | | |
| | | | | | | | | | | | | | (alpha-1 | also has a | | | | |
| | | | | | | | | | | | | | protease inhibitor | moderate | | | | |
| | | | | | | | | | | | | | 1) (alpha-1- | affinity for | | | | |
| | | | | | | | | | | | | | for plasmin and | | | | | |
| | | | | | | | | | | | | | thrombin. | | | | | |
| D30782 | 1.00 | 2.46 | −0.98 | 0.66 | −1.36 | 0.17 | −2.84 | 0.63 | −1.85 | 0.18 | epiregulin; Ereg | D30782 | | | | | | |
| W97690 | 1.00 | 1.87 | −3.28 | 1.59 | −1.46 | 0.19 | −1.78 | 0.22 | −1.97 | 0.28 | | W97690 | | | | | | |
| Msa.40750.0 | 1.00 | 1.20 | −2.95 | 0.51 | −1.94 | 0.71 | −1.81 | 0.34 | −2.05 | 0.38 | polypyrimidine tract | AA155318 | | | | | | |
| | | | | | | | | | | | binding protein 2; | | | | | | | |
| | | | | | | | | | | | Ptb2-pending | | | | | | | |
| AF030001E | 1.00 | −2.22 | −2.62 | 1.21 | −0.40 | 1.83 | −2.62 | 0.67 | −2.06 | 2.03 | Cosmid sequence | AF030001 | | | | EST; | | |
| NETNX | | | | | | | | | | | (>200K) | | | | | Unknown | | |
| Z22661 | 1.00 | 2.15 | −1.65 | 0.98 | −0.43 | 1.50 | −1.72 | 0.13 | −2.11 | 0.20 | apolipoprotein CI; | Z22661 | apolipoprotein c-i | appears to modulate | 7 4.0 cM | | | |
| | | | | | | | | | | | Apoc1 | | Precursor (apo-ci). | the interaction of apoe | | | | |
| | | | | | | | | | | | | | | with beta- migrating | | | | |
| | | | | | | | | | | | | | | v1d1 and inhibit | | | | |
| | | | | | | | | | | | | | | binding of beta-v1d1 to | | | | |
| | | | | | | | | | | | | | | the 1d1 receptor-related | | | | |
| | | | | | | | | | | | | | | protein | | | | |
| Msa.5470.0 | 1.00 | 1.04 | −1.96 | 0.78 | −1.15 | 0.14 | −3.08 | 0.53 | −2.14 | 0.38 | calsequestrin 1; | W11481 | calsequestrin, | calsequestrin is a high- | | Metabolic | | |
| | | | | | | | | | | | Casq1 | | skeletal muscle | capacity, moderate | | | | |
| | | | | | | | | | | | | | isoform precursor. | affinity, calcium- | | | | |
| | | | | | | | | | | | | | | binding protein and | | | | |
| | | | | | | | | | | | | | | thus acts as an internal | | | | |
| | | | | | | | | | | | | | | calcium store in | | | | |
| | | | | | | | | | | | | | | muscle. the release of | | | | |
| | | | | | | | | | | | | | | calcium bound to | | | | |
| | | | | | | | | | | | | | | calsequestrin through | | | | |
| | | | | | | | | | | | | | | a calcium release | | | | |
| | | | | | | | | | | | | | | channel triggers | | | | |
| | | | | | | | | | | | | | | muscle contraction. | | | | |
| | | | | | | | | | | | | | | binds 4 to 5 moles of | | | | |
| | | | | | | | | | | | | | | calcium. also binds | | | | |
| Msa.43184.0 | 1.00 | 1.45 | −2.47 | 0.76 | −1.34 | 0.21 | −5.16 | 1.47 | −2.20 | 1.26 | myosin, heavy | K00988 | | | 11 35.0 cM | Structural | | |
| | | | | | | | | | | | polypeptide 4, | | | | | Protein | | |
| | | | | | | | | | | | skeletal muscle; | | | | | | | |
| | | | | | | | | | | | Myh4 | | | | | | | |
| M32486 | 1.00 | −1.01 | −1.21 | 0.76 | −1.29 | 0.08 | −2.24 | 0.65 | −2.22 | 0.05 | hypothetical | M32486 | | geneseqn:q14534 lov | | Patented; | | |
| | | | | | | | | | | | protein | | | gene (cdna 19.5) - new | | Novel | | |
| | | | | | | | | | | | 19.5; p19.5 | | | recombinant | | | | |
| | | | | | | | | | | | | | | polypeptide | | | | |
| | | | | | | | | | | | | | | comprising a t-cell | | | | |
| | | | | | | | | | | | | | | protein - used to | | | | |
| | | | | | | | | | | | | | | regulate t-cell | | | | |
| | | | | | | | | | | | | | | development and | | | | |
| | | | | | | | | | | | | | | tumorigenic | | | | |
| | | | | | | | | | | | | | | phenotype and to | | | | |
| | | | | | | | | | | | | | | block t-cell activation | | | | |
| | | | | | | | | | | | | | | in auto:immune | | | | |
| | | | | | | | | | | | | | | disease. patent held by | | | | |
| X90875 | 1.00 | 1.47 | −3.41 | 1.29 | −2.40 | 0.52 | −2.34 | 0.43 | −2.23 | 0.14 | fragile X mental | X90875 | | | | | | |
| | | | | | | | | | | | retardation gene, | | | | | | | |
| | | | | | | | | | | | autosomal homolog; | | | | | | | |
| | | | | | | | | | | | Fxr1h | | | | | | | |
| W11010 | 1.00 | 2.04 | −1.68 | 0.59 | −1.49 | 0.05 | −1.88 | 0.32 | −2.24 | 0.17 | region of homolgy | W11010 | | | | Regulatory | | |
| | | | | | | | | | | | to: cell division | | | | | | | |
| | | | | | | | | | | | cycle 4- | | | | | | | |
| | | | | | | | | | | | like: beige-like; | | | | | | | |
| U30840 | 1.00 | 1.36 | −1.71 | 0.55 | −1.49 | 0.32 | −1.88 | 0.17 | −2.25 | 0.09 | voltage-dependent | U30840 | voltage-dependent | forms a channel | 11 29.0 cM | | | |
| | | | | | | | | | | | anion channel 1; | | anion-selective | through the | | | | |
| | | | | | | | | | | | Vdac1 | | channel protein 1 | mitochomdrial outer | | | | |
| | | | | | | | | | | | | | (mvdac1) | membrane | | | | |
| | | | | | | | | | | | | | (mvdac5) | that allows | | | | |
| | | | | | | | | | | | | | (outer | diffusion of small | | | | |
| | | | | | | | | | | | | | mitochondral | hydrophilic | | | | |
| | | | | | | | | | | | | | membrane protein | molecules the | | | | |
| | | | | | | | | | | | | | porin 1). | channel adopts an | | | | |
| | | | | | | | | | | | | | | open | | | | |
| | | | | | | | | | | | | | | conformation at | | | | |
| | | | | | | | | | | | | | | low or zero | | | | |
| | | | | | | | | | | | | | | membrane | | | | |
| | | | | | | | | | | | | | | potential and a | | | | |
| | | | | | | | | | | | | | | closed | | | | |
| | | | | | | | | | | | | | | conformation at | | | | |
| | | | | | | | | | | | | | | potentials above | | | | |
| | | | | | | | | | | | | | | 3-4 mv. the open | | | | |
| | | | | | | | | | | | | | | state has a weak | | | | |
| | | | | | | | | | | | | | | anion selectivity | | | | |
| | | | | | | | | | | | | | | whereas the | | | | |
| | | | | | | | | | | | | | | closed state is | | | | |
| | | | | | | | | | | | | | | cation- | | | | |
| J03398 | 1.00 | −1.60 | −2.11 | 0.74 | −1.32 | 0.07 | −4.66 | 0.85 | −2.29 | 1.31 | P glycoprotein 2; | J03398 | multidrug | mdr gene | 5 1.0 cM | Cell Surface | | |
| | | | | | | | | | | | Pgy2 | | resistance | encoding a | | Protein | | |
| | | | | | | | | | | | | | protein 2(p- | multidrug | | | | |
| | | | | | | | | | | | | | glycoprotein 2). | resistance | | | | |
| | | | | | | | | | | | | | | protein mrna | | | | |
| X61433 | 1.00 | 1.06 | −1.90 | 0.33 | −1.48 | 0.10 | −2.09 | 0.36 | −2.30 | 0.12 | ATPase, Na+/K+ | X61433 | sodium/ | expressed in | 1 86.8 cM | Cell Surface | | |
| | | | | | | | | | | | tranaporting, beta | | potassium | brain, kidney, | | Protein | | |
| | | | | | | | | | | | 1 polypeptide; | | transporting | lung, testis, and | | | | |
| | | | | | | | | | | | Atp1b1 | | aptase- | heart, not found | | | | |
| | | | | | | | | | | | | | beta-1-chain | in the liver. | | | | |
| | | | | | | | | | | | | | (sodium/ | expression occurs | | | | |
| | | | | | | | | | | | | | potassium | in pre-b | | | | |
| | | | | | | | | | | | | | dependent aptase | lymphocytes, | | | | |
| | | | | | | | | | | | | | beta-1 subunit). | resting b cells | | | | |
| | | | | | | | | | | | | | | in the bone | | | | |
| | | | | | | | | | | | | | | marrow, pre-t | | | | |
| | | | | | | | | | | | | | | cells, and | | | | |
| | | | | | | | | | | | | | | mature | | | | |
| | | | | | | | | | | | | | | tymocytes. | | | | |
| | | | | | | | | | | | | | | mitogen- | | | | |
| | | | | | | | | | | | | | | stimulated t and | | | | |
| | | | | | | | | | | | | | | b cells | | | | |
| Msa.2879.0 | 1.00 | 1.32 | −2.74 | 1.40 | −1.53 | 0.12 | −2 62 | 0.14 | −2.31 | 0.27 | transducer of- | D78382 | tob protein | anti- | | | | |
| | | | | | | | | | | | ErbB- | | (transducer of | proliferative | | | | |
| | | | | | | | | | | | 2.1; Tob1 | | erbb-2). | protein that | | | | |
| | | | | | | | | | | | | | | interacts with | | | | |
| | | | | | | | | | | | | | | the erbb-2 | | | | |
| | | | | | | | | | | | | | | receptor | | | | |
| | | | | | | | | | | | | | | tyrosine kinase | | | | |
| | | | | | | | | | | | | | | may physically | | | | |
| | | | | | | | | | | | | | | and/or | | | | |
| | | | | | | | | | | | | | | functionally | | | | |
| | | | | | | | | | | | | | | interact with | | | | |
| | | | | | | | | | | | | | | protein- | | | | |
| | | | | | | | | | | | | | | tyrosine kinase | | | | |
| | | | | | | | | | | | | | | receptors (by | | | | |
| | | | | | | | | | | | | | | similarity) | | | | |
| X06115 | 1.00 | 1.44 | −1.93 | 0.73 | −1.54 | 0.07 | −1.83 | 0.07 | −2.31 | 0.24 | cadherin 1; Cdh1 | X06115 | epithelial-cadherin | cadherins are calcium | 8 53.3 cM | | | |
| | | | | | | | | | | | | | precursor (e- | dependent cell | | | | |
| | | | | | | | | | | | | | cadherin) | adhesion proteins. | | | | |
| | | | | | | | | | | | | | (uvomorulin)(arc- | they preferentially | | | | |
| | | | | | | | | | | | | | 1). | interact with | | | | |
| | | | | | | | | | | | | | | themselves in a | | | | |
| | | | | | | | | | | | | | | homophilic manner in | | | | |
| | | | | | | | | | | | | | | connecting cells; | | | | |
| | | | | | | | | | | | | | | cadherins may thus | | | | |
| | | | | | | | | | | | | | | contribute to the | | | | |
| | | | | | | | | | | | | | | sorting of | | | | |
| | | | | | | | | | | | | | | heterogeneous cell | | | | |
| Msa.3250.0 | 1.00 | 1.14 | −4.21 | 2.32 | −1.75 | 0.48 | −1.66 | 0.34 | −2.32 | 0.40 | histidyl tRNA | U39473 | histidyl-trna | | | | | |
| | | | | | | | | | | | synthetase; Hars | | synthetase (ec | | | | | |
| | | | | | | | | | | | | | | 6.1.1.21)(histidine | | | | |
| | | | | | | | | | | | | | | trna ligase) (hisrs). | | | | |
| Msa.29324.0 | 1.00 | 1.63 | −1.48 | 1.37 | −1.32 | 0.26 | −2.00 | 0.18 | −2.33 | 0.11 | | AA08019 | | | | | | |
| Msa 4067.0 | 1.00 | 1.73 | −2.15 | 0.61 | −0.72 | 1.74 | −0.78 | 1.03 | −2.34 | 0.57 | Sip1? | AA003876 | | splicing factor (homo | | Unknown | | |
| | | | | | | | | | | | | | | sapiens) | | | | |
| Msa.5481.0 | 1.00 | 1.28 | −2.81 | 1.61 | −1.56 | 0.02 | −2.00 | 0.09 | −2.35 | 0.13 | annexin A8; | AA060106 | | | 14 13.0 cM | EST; | | |
| | | | | | | | | | | | Anxa8 | | | | | Unknown | | |
| AB000713 | 1.00 | −0.50 | −1.12 | 1.24 | 1.23 | 0.05 | −1.86 | 0.34 | −2.35 | 0.72 | claudin 4; Cldn4 | AB000713 | claudin-4 | a 4 transmembrane | 5 75.0 cM | Cell Surface | | |
| | | | | | | | | | | | | | (clostridium | domain protein that is | | Protein | | |
| | | | | | | | | | | | | | perfringens | a novel component of | | | | |
| | | | | | | | | | | | | | enterotoxin | tight junction strands | | | | |
| | | | | | | | | | | | | | receptor) (cpc- | of liver and kidney. | | | | |
| | | | | | | | | | | | | | receptor)(cpe-r). | | | | | |
| AA615066 | 1.00 | 2.07 | −4.34 2 32 | −0.57 | 1.61 | −1.58 | 0.35 | −2.38 | 0.54 | | AA61506 | | | | | | | |
| Msa.39064.0 | 1.00 | 1.13 | −2.50 | 1.11 | −1.58 | 0.22 | −2.58 | 0.28 | −2.39 | 0.18 | titin(scries elastic | AA14531 | | titin, giant sarcomeric | | Structural | | |
| | | | | | | | | | | | element of striated | | | protein, extending | | Protein | | |
| | | | | | | | | | | | muscle) | | | from the m line to the | | | | |
| | | | | | | | | | | | | | | z line of straited | | | | |
| | | | | | | | | | | | | | | muscle sarcomere, | | | | |
| | | | | | | | | | | | | | | essential in the | | | | |
| | | | | | | | | | | | | | | temporal and spatial | | | | |
| | | | | | | | | | | | | | | control of the | | | | |
| | | | | | | | | | | | | | | assembly of the highly | | | | |
| | | | | | | | | | | | | | | ordered sarcomeres of | | | | |
| | | | | | | | | | | | | | | striated muscles | | | | |
| M63170 | 1.00 | 1.06 | −1.12 | 1.18 | 1.12 | 0.11 | 1.13 | 0.06 | −2.40 | 0.09 | cryatallin, alpha 2; | M63170 | alpha crystallin b | expressed not | 9 29.0 cM | Signal | | |
| | | | | | | | | | | | Crya2 | | chain (alpha(b)- | only in lens, | | Transduction | | |
| | | | | | | | | | | | | | crystallin) (p23). | but in significant | | | | |
| | | | | | | | | | | | | | | amounts in heart | | | | |
| | | | | | | | | | | | | | | skeletal muscle | | | | |
| | | | | | | | | | | | | | | kidney, and lung; | | | | |
| | | | | | | | | | | | | | low levels in | | | | | |
| | | | | | | | | | | | | | | brain, and | | | | |
| | | | | | | | | | | | | | | “”alpha2 | | | | |
| | | | | | | | | | | | | | | crystallin | | | | |
| | | | | | | | | | | | | | | functions in | | | | |
| | | | | | | | | | | | | | | activating | | | | |
| | | | | | | | | | | | | | | myogenic | | | | |
| | | | | | | | | | | | | | | differentiation | | | | |
| | | | | | | | | | | | | | | and cardiac | | | | |
| | | | | | | | | | | | | | | membranes | | | | |
| AA285502 | 1.00 | −1.26 | −1.99 | 1.23 | −0.49 | 1.51 | −3.95 | 0.99 | −2.40 | 0.29 | receptor | AA285502 | | | | Regulatory | | |
| | | | | | | | | | | | (calcitonin) | | | | | | | |
| | | | | | | | | | | | activity | | | | | | | |
| | | | | | | | | | | | modifying | | | | | | | |
| | | | | | | | | | | | protein 1; Ramp1 | | | | | | | |
| Msa.5939.0 | 1.00 | 1.97 | −1.86 | 0.67 | −1.45 | 0.15 | −1.76 | 0.45 | −2.40 | 0.40 | GENESEQN: | W12756 | | | | Patented, | | |
| | | | | | | | | | | | V7426(3-5) | | | | | Novel | | |
| | | | | | | | | | | | Human heart | | | | | | | |
| | | | | | | | | | | | muscle specific | | | | | | | |
| | | | | | | | | | | | cDNA(s)#(1-3). | | | | | | | |
| U77039 | 1.00 | −1.31 | −1.62 | 0.95 | −1.24 | 0.03 | −1.49 | 0.15 | −2.40 | 0.12 | four and a half | U77039 | | both fh11 and | X A6-A7.1 | Regulatory | | |
| | | | | | | | | | | | LIM domains 1; | | | fh13 were | | | | |
| | | | | | | | | | | | Fh11 | | | expressed in a | | | | |
| | | | | | | | | | | | | | | number of | | | | |
| | | | | | | | | | | | | | | skeletal muscles | | | | |
| | | | | | | | | | | | | | | while fh12 was | | | | |
| | | | | | | | | | | | | | | expressed at high | | | | |
| | | | | | | | | | | | | | | levels in cardiac | | | | |
| X16490 | 1.00 | 1.84 | −2.02 | 0.95 | −1.76 | 0.18 | −2.40 | 0.30 | −2.42 | 0.24 | plasminogen | X16490 | plasminogen | pai-2 inhibits | 1 61.1 cM | | | |
| | | | | | | | | | | | activator | | activator | urokinase-type | | | | |
| | | | | | | | | | | | inhibitor, type II; | | inhibitor-2, | plasminogen | | | | |
| | | | | | | | | | | | Planh2 | | macrophage | activator. | | | | |
| | | | | | | | | | | | | | (pai-2). | the monocyte | | | | |
| | | | | | | | | | | | | | derived pai-2 is | | | | | |
| | | | | | | | | | | | | | distinct from the | | | | | |
| | | | | | | | | | | | | | endothelial cell- | | | | | |
| | | | | | | | | | | | | | derived pai-1 | | | | | |
| X81584 | 1.00 | −1.26 | −1.83 | 0.70 | −1.45 | 0.01 | −2.63 | 0.63 | −2.42 | 0.16 | insulin-like | X81584 | insulin-like | igf-binding proteins | | Cytokine | | |
| | | | | | | | | | | | growth factor | | growth factor | prolong the half-life of | | | | |
| | | | | | | | | | | | binding protein | | binding protein | the igfs and have been | | | | |
| | | | | | | | | | | | 6; Igfbp6 | | 6 precursor | shown to either inhibit | | | | |
| | | | | | | | | | | | | | (igfbp-6)(ibp-6) | or stimulate the | | | | |
| | | | | | | | | | | | | | (igf binding | growth promoting | | | | |
| | | | | | | | | | | | | | protein 6). | effects of the igfs on | | | | |
| | | | | | | | | | | | | | cell culture, they alter | | | | | |
| | | | | | | | | | | | | | the interaction of igfs | | | | | |
| | | | | | | | | | | | | | with their cell surface | | | | | |
| | | | | | | | | | | | | | receptors | | | | | |
| Msa.8234.0 | 1.00 | 1.27 | −5.73 | 4.82 | −1.31 | 0.12 | −2.15 | 0.02 | −2.43 | 0.11 | annexin A8; | AA032354 | | | 14 13.0 cM | | | |
| | | | | | | | | | | | Anxa8 | | | | | | | |
| Msa.570.0 | 1.00 | 1.28 | −3.02 | 2.47 | −0.25 | 1.42 | −2.54 | 0.82 | −2.43 | 0.44 | gap junction | M81445 | gap junction beta 2 | one gap junction | 14 21.0 cM | | | |
| | | | | | | | | | | | membrane channel | | protein (connexin | consists of a cluster of | | | | |
| | | | | | | | | | | | protein beta 2; | | 26) (ex26). | closely packed pairs of | | | | |
| | | | | | | | | | | | Gjb2 | | | transmembrane | | | | |
| | | | | | | | | | | | | | | channels, the | | | | |
| | | | | | | | | | | | | | | connexons, through | | | | |
| | | | | | | | | | | | | | | which amterials of low | | | | |
| | | | | | | | | | | | | | | mw diffuse from one | | | | |
| | | | | | | | | | | | | | | cell to a neighboring | | | | |
| | | | | | | | | | | | | | | celll | | | | |
| C77662 | 1.00 | 1.30 | −2.44 | 0.87 | −1.40 | 0.22 | −2.48 | 0.29 | −2.44 | 0.36 | | C77662 | | | | | | |
| Msa.3940.0 | 1.00 | −1.09 | −2.21 | 0.57 | −1.72 | 0 18 | −2.20 | 0.33 | −2.44 | 0.12 | Rev-ErbA-alpha | W13191 | | most similar in | | Receptor | | |
| | | | | | | | | | | | protein; rat | | | structure to the thyroid | | | | |
| | | | | | | | | | | | | | | hormone receptor (c- | | | | |
| | | | | | | | | | | | | | | erba) and the retinoic | | | | |
| | | | | | | | | | | | | | | acid receptor, but it | | | | |
| | | | | | | | | | | | | | | does not bind either | | | | |
| | | | | | | | | | | | | | | thyroid hormone or | | | | |
| | | | | | | | | | | | | | | retinoic acid. the mrna | | | | |
| | | | | | | | | | | | | | | encoding rev-erb | | | | |
| | | | | | | | | | | | | | | alpha is present in | | | | |
| | | | | | | | | | | | | | | many tissues and is | | | | |
| | | | | | | | | | | | | | | particularly a | | | | |
| AA690434 | 1.00 | 1.12 | −1.86 | 0.32 | −1.84 | 0.50 | −2.52 | 0.38 | −2.45 | 0.11 | | AA690434 | | | | | | |
| M72414 | 1.00 | 1.68 | −4.09 | 1.85 | −2.21 | 0.09 | −2.62 | 0.48 | −2.47 | 0.24 | microtubule- | M72414 | microtubule- | non-neuronal | 9 58.0 cM | | | |
| | | | | | | | | | | | associated protein | | associated protein | microtubule- | | | | |
| | | | | | | | | | | | 4; Mtap4 | | 4. | associated protein; | | | | |
| | | | | | | | | | | | | | | promotes microtubule | | | | |
| Msa.799.0 | 1.00 | −1.57 | −3.69 | 1.19 | −2.69 | 0.73 | −2.24 | 0.25 | −2.48 | 0.30 | interferon-related | J00424 | interferon-related | could play a role in | | Cytokine | | |
| | | | | | | | | | | | developmental | | developmental | regulating gene | | | | |
| | | | | | | | | | | | regulator 1; Ifrd1 | | regulator 1 (nerve | activity in the | | | | |
| | | | | | | | | | | | | | growth factor- | proliferative and/or | | | | |
| | | | | | | | | | | | | | inducible protein | differentiative | | | | |
| | | | | | | | | | | | | | pc4) (tpa induced | pathways induced by | | | | |
| | | | | | | | | | | | | | sequence 7) (tis7 | ngf. may be an | | | | |
| | | | | | | | | | | | | | protein). | autocrine factor that | | | | |
| | | | | | | | | | | | | | | attenuates or amplifies | | | | |
| | | | | | | | | | | | | | | the initial ligand | | | | |
| W29651 | 1.00 | 1.11 | −4.23 | 2.14 | −1.98 | 0.01 | −2.48 | 0.34 | −2.48 | 0.30 | | W29651 | | | | | | |
| AA688835 | 1.00 | −1.76 | −3.43 | 1.28 | −1.97 | 0.08 | −2.48 | 0.61 | −2.51 | 0.10 | Unknown | AA688835 | | | | EST; | | |
| | | | | | | | | | | | | | | | | Unknown | | |
| AJ001118 | 1.00 | 1.17 | −2.97 | 0.97 | −2.10 | 0.28 | −2.89 | 0.25 | −2.56 | 0.14 | monoglyceride | AJ001118 | | | 3 | | | |
| | | | | | | | | | | | lipase; Mg11 | | | | | | | |
| Msa.19265.0 | 1.00 | 2.33 | −2.61 | 0.87 | −1.88 | 0.75 | −1.96 | 0.52 | −2.57 | 0.53 | golgi autoantigen, | AA009086 | | | | | | |
| | | | | | | | | | | | golgin subfamily a, 4; | | | | | | | |
| | | | | | | | | | | | Golga4 | | | | | | | |
| AF026489 | 1.00 | 1.67 | −2.26 | 0.77 | −1.75 | 0.32 | −2.22 | 0.24 | −2.62 | 0.19 | beta-spectrin 3; | AF026489 | | | 19 0.0 cM | | | |
| | | | | | | | | | | | Spnb3 | | | | | | | |
| Msa.28719.0 | 1.00 | 1.54 | −3.87 | 1.00 | −3.39 | 1.49 | −2.22 | 0.10 | −2.65 | 0.05 | | AA072611 | | | | | | |
| Msa.9757.0 | 1.00 | 1.17 | −7.61 | 6.54 | −1.66 | 0.42 | −2.17 | 0.25 | −2.67 | 0.19 | basic transcription | AA014295 | | | | | | |
| | | | | | | | | | | | element binding | | | | | | | |
| | | | | | | | | | | | protein 2: K1f5 | | | | | | | |
| X97986 | 1.00 | 1.11 | −1.97 | 1.84 | −1.42 | 0.40 | −3.01 | 1.27 | −2.71 | 0.34 | desmocollin 1; | X97986 | desmocollin 1a/1b | component of | | | | |
| | | | | | | | | | | | Dsc1 | | precursor. | intercellular | | | | |
| | | | | | | | | | | | | | | desmosome junctions. | | | | |
| | | | | | | | | | | | | | | involved in the | | | | |
| | | | | | | | | | | | | | | interaction of plaque | | | | |
| | | | | | | | | | | | | | | proteins and | | | | |
| | | | | | | | | | | | | | | intermediate filamnets | | | | |
| | | | | | | | | | | | | | | mediating cell-cell | | | | |
| | | | | | | | | | | | | | | adhesion. may | | | | |
| | | | | | | | | | | | | | | contribute to | | | | |
| | | | | | | | | | | | | | | epidermal cell | | | | |
| | | | | | | | | | | | | | | positioning | | | | |
| | | | | | | | | | | | | | | (stratification) by | | | | |
| | | | | | | | | | | | | | | mediating differential | | | | |
| | | | | | | | | | | | | | | adhesiveness between | | | | |
| | | | | | | | | | | | | | | cells that express | | | | |
| | | | | | | | | | | | | | | different isoforms. | | | | |
| | | | | | | | | | | | | | | linked to the | | | | |
| Msa 32581.0 | 1.00 | 1.41 | −2.38 | 1.08 | −1.25 | 0.21 | −2.58 | 0.43 | −2.72 | 0.11 | solute carrier | AA107658 | adp,atp carrier | carries adenosine | 8 26.0 cM | Other | | |
| | | | | | | | | | | | family 25 | | protein, | triphosphate (atp) | | | | |
| | | | | | | | | | | | (mitochondrial | | heart/skeletal | from the | | | | |
| | | | | | | | | | | | carrier; adenine | | muscle isoform t1 | mitochondrial matrix | | | | |
| | | | | | | | | | | | nucleotide | | (adp/atp | into the | | | | |
| | | | | | | | | | | | translocator), | | translocase 1) | intermembrane space | | | | |
| | | | | | | | | | | | member 4; | | (adenine | and the diphosphate | | | | |
| | | | | | | | | | | | Slc25a4 | | nucleotide | (adp) in the reverse | | | | |
| | | | | | | | | | | | | | translocator 1) | | | | | |
| | | | | | | | | | | | | | 1) ant | | | | | |
| Msa.450.0 | 1.00 | −1.56 | −3.38 | 1.69 | −1.73 | 0.11 | −4.22 | 2.40 | −2.74 | 0.20 | adipsin; Adn | W36455 | complement | a serine protease | 10 43.0 cM | Proteolytic | | |
| | | | | | | | | | | | | | factor d precursor | synthesized principally | | | | |
| | | | | | | | | | | | | | (ec 3.4.21.46) (c3 | in adipose tissue, but | | | | |
| | | | | | | | | | | | | | convertase | also by sciatic nerve . . . | | | | |
| | | | | | | | | | | | | | activator) | adipsin in suppressed | | | | |
| | | | | | | | | | | | | | (properdin | (more than 1-fold) in | | | | |
| | | | | | | | | | | | | | factor d) | genetically obese mice | | | | |
| | | | | | | | | | | | | | protein | | | | | |
| | | | | | | | | | | | | | adinocyte) | | | | | |
| Msa.11196.0 | 1.00 | 1.31 | −1.76 | 0.72 | −1.74 | 0.44 | −1 70 | 0.37 | −2.74 | 0.24 | | W50088 | | | | | | |
| U06670 | 1.00 | 1.16 | −2.84 | 1.03 | −1.65 | 0.01 | −3.54 | 1.46 | −2.75 | 0.44 | very low density | U06670 | very low-density | binds v1d1 and | 19 20.0 cM | Receptor | | |
| | | | | | | | | | | | lipoprotein | | lipoprotein | transports it into cells | | | | |
| | | | | | | | | | | | receptor; | | receptor | by endocytosis. in | | | | |
| | | | | | | | | | | | Vldlr | | precursor (vldl | order to be | | | | |
| | | | | | | | | | | | | | receptor). | internalized, the | | | | |
| | | | | | | | | | | | | | | receptor-ligand | | | | |
| | | | | | | | | | | | | | | complexes must first | | | | |
| | | | | | | | | | | | | | | cluster into clathrin- | | | | |
| | | | | | | | | | | | | | | coated pits | | | | |
| Msa.23977.0 | 1.00 | 1.39 | −2.52 | 1.29 | −1.22 | 0.04 | −2.57 | 0.68 | −2.76 | 0.26 | EST | W07946 | | geneseqn:z9721 | | | | |
| | | | | | | | | | | | | | | human secreted | | | | |
| | | | | | | | | | | | | | | protein gene 3 cdna | | | | |
| | | | | | | | | | | | | | | clone hwhgu54, seq | | | | |
| | | | | | | | | | | | | | | id no: 13, new isolated | | | | |
| | | | | | | | | | | | | | | human genes and the | | | | |
| | | | | | | | | | | | | | | secreted treatment of | | | | |
| | | | | | | | | | | | | | | e.g. cancers | | | | |
| W71831 | 1.00 | 1.37 | −5.51 | 3.22 | −1.70 | 0.18 | −3.58 | 1.40 | −2.76 | 0.36 | histone | W71831 | | | | | | |
| | | | | | | | | | | | deacetylase 5; | | | | | | | |
| | | | | | | | | | | | Hdac5 | | | | | | | |
| U12785 | 1.00 | 1.04 | −2.41 | 0.64 | −1.96 | 0.28 | −2.62 | 0.39 | −2.77 | 0.07 | alcohol | U12785 | aldehyde | aldhs play a major role | 11 34.25 | | | |
| | | | | | | | | | | | dehydrogenase | | dehydrogenase, | in the detoxification of | cM | | | |
| | | | | | | | | | | | family 3, | | dimeric nadp- | alcohol-derived | | | | |
| | | | | | | | | | | | subfamily A1; | | preferring (ec | acetaldehyde, they are | | | | |
| | | | | | | | | | | | Aldh3a1 | | 1.2.1.5) (aldh class | involved in the | | | | |
| | | | | | | | | | | | | | 3) (dioxin-inducible | metabolism of | | | | |
| | | | | | | | | | | | | | aldehyde | corticosteroids, | | | | |
| | | | | | | | | | | | | | dehydrogenase- | biogenic amines, | | | | |
| | | | | | | | | | | | | | 3).,fatty aldehyde | neurotransmitters, and | | | | |
| | | | | | | | | | | | | | dehydrogenase (ec | lipid peroxidation, this | | | | |
| | | | | | | | | | | | | | 1.2.1.3) (aldehyde | protein preferentially | | | | |
| | | | | | | | | | | | | | dehydrogenase, | oxidizes aromatic | | | | |
| | | | | | | | | | | | | | microsomal) (aldh | aldehyde substrates. it | | | | |
| | | | | | | | | | | | | | class 3). | may play a role in the | | | | |
| | | | | | | | | | | | | | | oxidation of toxic | | | | |
| AA462409 | 1.00 | 1.14 | −1.61 | 1.30 | −1.49 | 0.25 | −2.23 | 0.31 | −2.81 | 0.04 | Unknown | AA462409 | | | | EST; | | |
| | | | | | | | | | | | | | | | | Unknown | | |
| Msa.4575.0 | 1.00 | −1.01 | −2.05 | 1.68 | −1.47 | 0.36 | −2.53 | 0.73 | −2.81 | 0.20 | EST | AA065868 | | geneseqn:z56885 | | | | |
| | | | | | | | | | | | | | | human abpsap1 | | | | |
| | | | | | | | | | | | | | | polypeptide encoding | | | | |
| | | | | | | | | | | | | | | est derived sequence. | | | | |
| | | | | | | | | | | | | | | new polypeptides of | | | | |
| | | | | | | | | | | | | | | prosaposin family, | | | | |
| | | | | | | | | | | | | | | ntagonist and | | | | |
| | | | | | | | | | | | | | | inhibitors for | | | | |
| | | | | | | | | | | | | | | treatment | | | | |
| Msa.16748.0 | 1.00 | 2.17 | −2.91 | 1.31 | −1.55 | 0.39 | −2.81 | 0.81 | −2.83 | 0.59 | | W78443 | | | | | | |
| Msa.3237.0 | 1.00 | 1.01 | −2.22 | 1.69 | −1.64 | 0.57 | −1.49 | 0.07 | −2.83 | 0.29 | four and a half | W14830 | | fh11 and fh13 were | X A6-A7.1 | Regulatory | | |
| | | | | | | | | | | | LIM domains 1; | | | expressed in a number | | | | |
| | | | | | | | | | | | Fhl1 | | | expressed in a number | | | | |
| | | | | | | | | | | | | | | of skeletal muscles | | | | |
| | | | | | | | | | | | | | | while fh12 was | | | | |
| | | | | | | | | | | | | | | expressed at high | | | | |
| | | | | | | | | | | | | | | levels in cardiac | | | | |
| | | | | | | | | | | | | | | muscle. may have an | | | | |
| | | | | | | | | | | | | | | involvement in muscle | | | | |
| | | | | | | | | | | | | | | development or | | | | |
| AA028770 | 1.00 | 0.02 | −2.96 | 1.22 | −0.34 | 1.56 | −2.74 | 0.63 | −2.96 | 1.02 | Cysteine Rich | AA028770 | | expressed in | | Regulatory | | |
| | | | | | | | | | | | protein 2, rat | | | differentiated vascular | | | | |
| | | | | | | | | | | | | | | smooth muscle cells. | | | | |
| | | | | | | | | | | | | | | during development | | | | |
| | | | | | | | | | | | | | | crp2/smlim expression | | | | |
| | | | | | | | | | | | | | | decreased in the heart | | | | |
| | | | | | | | | | | | | | | but remained high in | | | | |
| | | | | | | | | | | | | | | the vasculatore | | | | |
| M91236 | 1.00 | 1.29 | −2.27 | 1.21 | −1.61 | 0.53 | −2.93 | 1.20 | −2.96 | 0.27 | gap junction | M91236 | gap junction beta-5 | one gap junction | 4 57.5 cM | | | |
| | | | | | | | | | | | membrane channel | | protein (connexin | consists of a cluster of | | | | |
| | | | | | | | | | | | protein beta 5; | | 30.3) (cx30.3). | closely packed pairs of | | | | |
| | | | | | | | | | | | Gjb5 | | | transmembrane | | | | |
| | | | | | | | | | | | | | | channels, the | | | | |
| | | | | | | | | | | | | | | connexons, through | | | | |
| | | | | | | | | | | | | | | which materials of low | | | | |
| | | | | | | | | | | | | | | mw diffuse from one | | | | |
| | | | | | | | | | | | | | | cell to a neighboring | | | | |
| | | | | | | | | | | | | | | cell | | | | |
| W62701 | 1.00 | 1.71 | −3.09 | 2.19 | −0 68 | 0.74 | −2.36 | 0.14 | −2.88 | 0.52 | | W62701 | | | | | | |
| Msa.6594.0 | 1.00 | 1.58 | −1.24 | 1.11 | −0.37 | 1.53 | −4.56 | 2.63 | −2.92 | 0.36 | | W30612 | | | | | | |
| Msa.7275.0 | 1.00 | 1.19 | −1.82 | 1.28 | −1.28 | 0.03 | −2.67 | 0.32 | −2.95 | 0.31 | Mus musculus | W17917 | | | | | | |
| | | | | | | | | | | | phosphofructo- | | | | | | | |
| | | | | | | | | | | | kinase | | | | | | | |
| | | | | | | | | | | | 1A isozyme | | | | | | | |
| | | | | | | | | | | | (Pfka) | | | | | | | |
| Msa.26512.0 | 1.00 | 1.31 | −1.53 | 1.17 | −1.38 | 0.29 | −2.28 | 0.39 | −3.01 | 0.48 | tubulin alpha 8; | AA063914 | | | | | | |
| | | | | | | | | | | | Tuba8 | | | | | | | |
| X13135 | 1.00 | 1.12 | −4.53 | 1.73 | −2.16 | 0.14 | −3.08 | 0.44 | −3.06 | 0.45 | fatty acids synthase | X13135 | fatty acid synthase | fatty acid synthase | 11 72.0 cM | Metabolic | | |
| | | | | | | | | | | | synthase; Fasn | | synthase | (fas) catalyzes the last | | | | |
| | | | | | | | | | | | | | (ec 2.3.1.85) | step in the fatty acid | | | | |
| | | | | | | | | | | | | | [(includes: ec | biosynthetic pathway | | | | |
| | | | | | | | | | | | | | 2.3.1.38; ec | | | | | |
| | | | | | | | | | | | | | 2.3.1.39; ec | | | | | |
| | | | | | | | | | | | | | 2.3.1.41; ec | | | | | |
| | | | | | | | | | | | | | 1.1.1.100; ec | | | | | |
| | | | | | | | | | | | | | 4.2.1.61; ec | | | | | |
| | | | | | | | | | | | | | 1.3.1.10: ec | | | | | |
| Msa.3669.0 | 1.00 | −1.55 | −1.69 | 0.98 | −1.64 | 0.34 | −2.26 | 0.62 | −3.06 | 0.28 | est | W08486 | | | | EST; | | |
| | | | | | | | | | | | | | | | | Unknown | | |
| AA138388 | 1.00 | 1.25 | −2.10 | 0.49 | −1.90 | 0.24 | −1.97 | 0.25 | −3.07 | 0.63 | | AA138388 | | | | | | |
| U62295 | 1.00 | 1.09 | −3.22 | 1.18 | −1.83 | 0.38 | −2.99 | 0.47 | −3.07 | 0.10 | cytochrome P450, | U62295 | cytochrome p450 | | 4 46.5 cM | | | |
| | | | | | | | | | | | 2j6;Cyp2j6 | | 2j6(ec 1.14.14.1) | | | | | |
| | | | | | | | | | | | | | (cypiij6) | | | | | |
| | | | | | | | | | | | | | (arachidonic acid | | | | | |
| | | | | | | | | | | | | | epoxygenase). | | | | | |
| Msa.2753.0 | 1.00 | −1.18 | −5.10 | 2.45 | −1.92 | 0.61 | −2.80 | 0.31 | −3.08 | 0 53 | laminin, beta 2; | U43541 | laminin beta-2 | extracellular matrix | 9 60.0 cM | ECM | | |
| | | | | | | | | | | | Lamb2 | | chain precursor. | glycoproteins which | | (Matrix | | |
| | | | | | | | | | | | | | | are major components | | Prot) | | |
| | | | | | | | | | | | | | | of basement | | | | |
| | | | | | | | | | | | | | | membranes | | | | |
| AA734300 | 1.00 | 1.09 | −2.42 | 0.84 | −1.93 | 0.62 | −2.71 | 0.59 | −3.10 | 0.48 | Hypothetical | AA734300 | | | | Unknown | | |
| | | | | | | | | | | | protein | | | | | | | |
| | | | | | | | | | | | FL120171, human | | | | | | | |
| Msa.717.0 | 1.00 | −1.17 | −4.27 | 2.07 | −1.93 | 0.53 | −5.02 | 1.56 | −3.12 | 0.59 | glycerolphosphate | M13366 | glycerol-3- | belongs to the nad- | 15 56.8 cM | Metabolic | | |
| | | | | | | | | | | | dehydrogenase 1, | | phosphate | dependent glycerol-3 | | | | |
| | | | | | | | | | | | cytoplasmic adult; | | dehydrogenase | phosphate | | | | |
| | | | | | | | | | | | Gdc 1 | | [nad+], | dehydrogenase family. | | | | |
| | | | | | | | | | | | | | | cytoplasmic | | | | |
| | | | | | | | | | | | | | | (ec 1.1.1.8) | | | | |
| | | | | | | | | | | | | | | (gpd-c) | | | | |
| | | | | | | | | | | | | | | (gpdh-c) | | | | |
| AA409316 | 1.00 | 1.83 | −2.55 | 1.34 | −1.53 | 0.46 | −3.69 | 0.33 | −3.12 | 0.71 | | AA409316 | | | | | | |
| Msa.12516.0 | 1.00 | 1.38 | −2.89 | 1.39 | −1.30 | 0.19 | −2.89 | 0.42 | −3.12 | 0.54 | Unknown | W55004 | | | | Unknown | | |
| Msa.41264.0 | 1.00 | 1.77 | −5.60 | 2.99 | −1.66 | 0.56 | −7.59 | 2.64 | −3.17 | 1.80 | myosin heavy | AA162315 | | myh8 | | Structural | | |
| | | | | | | | | | | | chain EST to | | | | | Protein | | |
| L04678 | 1.00 | 1.47 | −3.65 | 1.95 | −1.38 | 0.37 | −1.80 | 0.40 | −3 18 | 0.26 | integrin beta 4; Itgb4 | L04678 | | | 11 76.0 cM | | | |
| Z22866 | 1.00 | 1.92 | −2.24 | 0.85 | −1.62 | 0.34 | −3.33 | 0.70 | −3.19 | 0.76 | myomesin 1; Myom1 | Z22866 | | | | | | |
| Msa.726.0 | 1.00 | 1.50 | −3.07 | 1.42 | −1.49 | 0.04 | −2.21 | 0.08 | −3.21 | 0.73 | glutathione-S- | W29265 | glutathione s- | liver enzyme; | 9 44.0 cM | Metabolic | | |
| | | | | | | | | | | | transferase, alpha 2 | | transferase gt41a | | | | | |
| | | | | | | | | | | | (Yc2); Gsta2 | | (ec 2.5.1.18) (gst | | | | | |
| | | | | | | | | | | | | | (class-alpha). | | | | | |
| U31510 | 1.00 | 1.18 | −4.17 | 2.06 | −2.06 | 0.23 | −1.40 | 1.32 | −3.21 | 0.39 | ADP- | U31510 | gpi-linked | poly[adp-ribose] | 7 50.0 cM | Metabolic | | |
| | | | | | | | | | | | ribosyltransferase | | nad(p)(+)- | polymerase modities | | | | |
| | | | | | | | | | | | 1; | arginine adp- | ribosyltransferase | various nuclear | | | | |
| | | | | | | | | | | | | Art1 | | ribosyltransferase | proteins by poly(adp- | | | |
| | | | | | | | | | | | | | precursor (ec | ribosyl)ation. the | | | | |
| | | | | | | | | | | | | | 2.4.2.31) (mono | modification is | | | | |
| | | | | | | | | | | | | | (adp | dependent on dna and | | | | |
| | | | | | | | | | | | | | ribosyl) | is involved in the | | | | |
| | | | | | | | | | | | | | transferase) | regulation of various | | | | |
| | | | | | | | | | | | | | (yac-1)., poly | important cellular | | | | |
| | | | | | | | | | | | | | [adp-ribose] | processes such as | | | | |
| | | | | | | | | | | | | | polymerase | differentiation | | | | |
| | | | | | | | | | | | | | (ec 2.4.2.30) | proliferation, and | | | | |
| | | | | | | | | | | | | | | (parp)(adprt) | tumor transformation | | | |
| | | | | | | | | | | | | | | (nad(+)adp- | and also in the | | | |
| | | | | | | | | | | | | | | ribosyltransfer- | regulation of the | | | |
| | | | | | | | | | | | | | | ase)(poly | molecular events | | | |
| | | | | | | | | | | | | | | [adp-ribose] | involved in the | | | |
| | | | | | | | | | | | | | | synthetase). | recovery of cell from | | | |
| Msa.3511.0 | 1.00 | 2.20 | −1.47 | 0.57 | 0.08 | 1.27 | −1.85 | 0.37 | −3.23 | 0.71 | aldolase 3, C | W53351 | fructose- | | 11 44.98 | | | |
| | | | | | | | | | | | isoform; Aldo3 | | bisphosphate | | cM | | | |
| | | | | | | | | | | | | | aldolase c (ec | | | | | |
| | | | | | | | | | | | | | 4.1.2.13) (brain-type | | | | | |
| | | | | | | | | | | | | | aldolase) | | | | | |
| | | | | | | | | | | | | | (fragment). | | | | | |
| AA691651 | 1.00 | 1.12 | −2.64 | 0.65 | −1.94 | 0.58 | −2.90 | 0.47 | −3.24 | 0.30 | EST; Unknown | AA691651 | | | | EST; | | |
| | | | | | | | | | | | | | | | | Unknown | | |
| X51905 | 1.00 | 1.55 | −5.30 | 3.66 | −1.51 | 0.09 | −2.88 | 0.28 | −3.28 | 0.36 | lactate | X51905 | 1-lactate | | 6 62.0 cM | Metabolic | | |
| | | | | | | | | | | | dehydrogenase 2, | | dehydrogenase h | | | | | |
| | | | | | | | | | | | B chain; Ldh2 | | chain | | | | | |
| | | | | | | | | | | | | | (ec 1.1.1.27) | | | | | |
| | | | | | | | | | | | | | (1dh-b) | | | | | |
| W15862 | 1.00 | 1 30 | −1.53 | 0.84 | −1.60 | 0.30 | −3.12 | 0.84 | −3.29 | 0.40 | uncoupling | W15862 | mitochondrial | blast of 12/99 = no | 7 50.0 cM | Intracellular | | |
| | | | | | | | | | | | protein 2, | | uncoupling | match | | Protein | | |
| | | | | | | | | | | | mitochondrial; | | protein | | | | | |
| | | | | | | | | | | | Ucp2 | | 2(ucp 2)(ucph). | | | | | |
| Msa.30092.0 | 1.00 | 1.23 | −8.99 | 6.81 | −1.44 | 0.05 | −5.46 | 1.82 | −3.29 | 0.54 | myosin, heavy | AA089202 | myosin heavy | muscle contraction. | 11 35.0 cM | | | |
| | | | | | | | | | | | polypeptide 3, | | fast skeletal muscle, | | | | | |
| | | | | | | | | | | | skeletal | | embryonic | | | | | |
| | | | | | | | | | | | muscle, | | (fragment) | | | | | |
| | | | | | | | | | | | embryonic: Myh3 | | | | | | | |
| Msa.23986.0 | 1.00 | −1.13 | −2.92 | 0.87 | −1.80 | 0.13 | −3.03 | 0.46 | −3.30 | 0.65 | EST to LTBP4 | W16389 | | latent transforming | | | | |
| | | | | | | | | | | | | | | growth factor 6% | | | | |
| | | | | | | | | | | | | | | nucleotide level | | | | |
| AA423082 | 1.00 | 1.23 | −2.92 | 0.98 | −1.73 | 0.72 | −2.72 | 0.71 | −3.37 | 0.72 | Unknown | AA423082 | | | | EST; | | |
| | | | | | | | | | | | | | | | | Unknown | | |
| Msa.21797.0 | 1.00 | 1.31 | −2.77 | 0.75 | −1.85 | 0.02 | −3.30 | 0.31 | −3.44 | 0.32 | ADP- | AA028701 | | | | Other | | |
| | | | | | | | | | | | ribosyltransferase | | | | | | | |
| | | | | | | | | | | | 3 (Art3) | | | | | | | |
| Msa.1286.0 | 1.00 | 1.60 | −3.15 | 1.51 | −2.15 | 0.66 | −6.37 | 2.47 | −3.45 | 1.64 | wingless-related | M89797 | wnt-4protein | may be an intracellular | 4 | | | |
| | | | | | | | | | | | MMTV integration | | precursor | signaling molecule | | | | |
| | | | | | | | | | | | site 4; Wnt4 | | | involved in | | | | |
| | | | | | | | | | | | | | | segmentation of the | | | | |
| | | | | | | | | | | | | | | forebrain. is likely to | | | | |
| | | | | | | | | | | | | | | signal over only few | | | | |
| | | | | | | | | | | | | | | cell diameters (by | | | | |
| | | | | | | | | | | | | | | similarity). seems to | | | | |
| | | | | | | | | | | | | | | be involved in kidney | | | | |
| | | | | | | | | | | | | | | development | | | | |
| X51829 | 1.00 | 1.04 | −4.58 | 2.18 | −2.40 | 0.50 | −2.90 | 0.28 | −3.48 | 0.39 | myeloid | X51829 | myeloid | | | Other | | |
| | | | | | | | | | | | differentiation | | differentation | | | | | |
| | | | | | | | | | | | primary response | | primary response | | | | | |
| | | | | | | | | | | | gene 116: Mvd116 | | protein myd116. | | | | | |
| Msa.3168.0 | 1.00 | 1.09 | −3.46 | 1.73 | −1.64 | 0.00 | −2.65 | 0.18 | −3.66 | 0.88 | gap junction | Z70023 | gap junction | one gap junction | 14 | | | |
| | | | | | | | | | | | membrane | | protein (connexin | consists of a cluster of | | | | |
| | | | | | | | | | | | channel | beta 6; | | 30) (cx30) | closely packed pairs of | | | |
| | | | | | | | | | | | protein beta 6; | | | transmembrane | | | | |
| | | | | | | | | | | | Gjb6 | | | channels, the | | | | |
| | | | | | | | | | | | | | | connexons, through | | | | |
| | | | | | | | | | | | | | | which materials of low | | | | |
| | | | | | | | | | | | | | | mw diffuse fron one | | | | |
| | | | | | | | | | | | | | | cell to a neighboring | | | | |
| | | | | | | | | | | | | | | cell | | | | |
| ET62740 | 1.00 | 2.18 | −2.68 | 0.86 | −2.03 | 0.73 | −2.43 | 0.60 | −3.69 | 0.95 | ankyrin 3, | ET62740 | | | 10 38.0 cM | | | |
| | | | | | | | | | | | epithelial; Ank3 | | | | | | | |
| M74495 | 1.00 | −1.28 | −1.86 | 1.37 | −1.22 | 0.02 | −2.60 | 0.64 | −3.70 | 0.25 | adenylosuccinate | M74495 | adenylosuccinate | plays an important | | | | |
| | | | | | | | | | | | synthetase 1, | | synthetase, | role in the de novo | | | | |
| | | | | | | | | | | | muscle; Adss1 | | muscle isozyme | pathway of purine | | | | |
| | | | | | | | | | | | | | (ec 6.3.4.4) | nucleotide | | | | |
| | | | | | | | | | | | | | (imp-aspartate | biosynthesis. | | | | |
| | | | | | | | | | | | | | ligase)(adss) | | | | | |
| | | | | | | | | | | | | | (ampsase). | | | | | |
| AA407234 | 1 00 | 1.93 | −2.78 | 0.47 | −2.39 | 1.02 | −3.71 | 1.15 | −3.74 | 0.79 | | AA407234 | | | | | | |
| U76618 | 1.00 | 1.16 | −1.51 | 1.02 | −1.52 | 0.24 | −1.60 | 0.21 | −3.77 | 0.45 | nebulin-related | U76618 | | specific to skeletal and | 10 53.25 | Structural | | |
| | | | | | | | | | | | anchoring protein; | | | cardiac muscle, not | cM | Protein | | |
| | | | | | | | | | | | Nrap | | | detected by northern | | | | |
| | | | | | | | | | | | | | | blot in non-muscle. | | | | |
| | | | | | | | | | | | | | | localized at | | | | |
| | | | | | | | | | | | | | | myotendinous junction | | | | |
| | | | | | | | | | | | | | | in mouse skeletal | | | | |
| | | | | | | | | | | | | | | muscle and | | | | |
| | | | | | | | | | | | | | | intercalated disc in | | | | |
| | | | | | | | | | | | | | | cardiac muscle. plays | | | | |
| | | | | | | | | | | | | | | a role in anchoring | | | | |
| | | | | | | | | | | | | | | terminal actin | | | | |
| AA717225 | 1.00 | −1.07 | −2.01 | 0.52 | −1.49 | 0.03 | −2.85 | 0.77 | −3.80 | 0 63 | | AA717225 | | | | | | |
| Msa.15880.0 | 1.00 | −5.84 | −2.18 | 1.33 | −1.49 | 0.24 | −2.09 | 0.30 | −3.81 | 0.73 | neuropeptide Y; | W70782 | | implicated in the | | Other | | |
| | | | | | | | | | | | Y: NPY | | | control of feeding and | | | | |
| | | | | | | | | | | | | | | in secretion | | | | |
| | | | | | | | | | | | | | | gonadotrophin-release | | | | |
| | | | | | | | | | | | | | | hormone | | | | |
| M81086 | 1.00 | 1.11 | −2.08 | 0.96 | −1.62 | 0.39 | −5.80 | 3.35 | −3.82 | 0.31 | tropomyosin 2, | M81086 | | an actin-associated | | Structural | | |
| | | | | | | | | | | | beta: Tpm2 | | | cytoskeletal protein, | | Protein | | |
| | | | | | | | | | | | | | | different isoforms | | | | |
| | | | | | | | | | | | | | | occur in skeletal | | | | |
| | | | | | | | | | | | | | | muscle and in smooth | | | | |
| | | | | | | | | | | | | | | muscle and nonmuscle | | | | |
| | | | | | | | | | | | | | | cells | | | | |
| AF026072 | 1.00 | −0.16 | −1.09 | 1.73 | −0.29 | 1.69 | −2.40 | 1.47 | −3.83 | 1.01 | hydroxysteroid | AF026072 | | | | | | |
| | | | | | | | | | | | sulfotransferase; | | | | | | | |
| | | | | | | | | | | | SULT2B | | | | | | | |
| C80836 | 1.00 | 1.45 | −3.34 | 1.10 | −2.41 | 0.17 | −6.22 | 1.90 | −3.83 | 1.08 | EST; unknown | C80836 | | | | EST; | | |
| | | | | | | | | | | | | | | | | Unknown | | |
| AA265119 | 1.00 | −2.16 | −2.71 | 0.67 | −2.32 | 0.55 | −6.91 | 3.49 | −3.90 | 0.53 | EST; Unknown | AA265119 | | | | EST; | | |
| | | | | | | | | | | | | | | | | Unknown | | |
| D42048 | 1.00 | −1.41 | −1.83 | 1.36 | −1.80 | 0.64 | −2.87 | 0.48 | −3.92 | 0.40 | squalene | D42048 | squalene | catalyzes the first | | | | |
| | | | | | | | | | | | epoxidase; Sqle | | monooxygenase | oxygenation step in | | | | |
| | | | | | | | | | | | | | (ec 1.14.99.7) | sterol biosynthesis and | | | | |
| | | | | | | | | | | | | | (squalene | is suggested to be one | | | | |
| | | | | | | | | | | | | | epoxidase)(se). | of the rate-limiting | | | | |
| | | | | | | | | | | | | | | enzymes in this | | | | |
| | | | | | | | | | | | | | | pathway | | | | |
| Mas400.0 | 1.00 | 1.24 | −4.47 | 1.71 | −2.24 | 0.01 | −4.10 | 0.65 | −3.98 | 0.71 | myosin heavy | M76601 | myosin heavy chain, | adult cardiac specific | 14 20.0 cM | Structural | | |
| | | | | | | | | | | | chain cardiac | | chain cardiac | isoform of myosin | | Protein | | |
| | | | | | | | | | | | muscle, adult; | | muscle alpha | heavy chain. (see | | | | |
| | | | | | | | | | | | Myhca | | isoform. | additional information | | | | |
| | | | | | | | | | | | | | | for regulation). | | | | |
| U76371 | 1.00 | 1.17 | −1.64 | 1.11 | −0.08 | 1.23 | −4.77 | 1.17 | −4.01 | 1.08 | CD8beta opposite | U76371 | | a transcribed gene | 6 30.5 cM | Other | | |
| | | | | | | | | | | | strand; Bop | | | designated as bop, is a | | | | |
| | | | | | | | | | | | | | | cd8-beta opposite | | | | |
| | | | | | | | | | | | | | | direction transcript. | | | | |
| | | | | | | | | | | | | | | detected in mouse | | | | |
| | | | | | | | | | | | | | | thymus only, and may | | | | |
| | | | | | | | | | | | | | | be limited to cd8+ t | | | | |
| | | | | | | | | | | | | | | cells | | | | |
| Msa.5248.0 | 1.00 | 1.53 | −3.03 | 1.51 | −1.52 | 0.11 | −3.41 | 0.56 | −4.02 | 0.44 | phosphofructo- | W11082 | | defects in pfkm are the | | Metabolic | | |
| | | | | | | | | | | | kinase 1A | | | cause of glycogen | | | | |
| | | | | | | | | | | | isozyme (Pfka) | | | storage disease vii | | | | |
| | | | | | | | | | | | | | | (gsd-vii) (also known | | | | |
| | | | | | | | | | | | | | | as tarui's disease); | | | | |
| | | | | | | | | | | | | | | a disease characterized | | | | |
| | | | | | | | | | | | | | | by exercise | | | | |
| | | | | | | | | | | | | | | intolerance with | | | | |
| | | | | | | | | | | | | | | associated nausea and | | | | |
| U15541 | 1.00 | 1.39 | −1.98 | 1.01 | −1.30 | 0 23 | −3.58 | 1.33 | −4.05 | 0.43 | cytochrome c | U15541 | cytochrome c | this protein is one of | 7 68.8 cM | Metabolic | | |
| | | | | | | | | | | | oxidase, subunit | | oxidase polypeptide | the nuclear-coded | | | | |
| | | | | | | | | | | | VIIIb; Cox8b | | viii-heart | polypeptide chains of | | | | |
| | | | | | | | | | | | | | precursor | cytochrome c oxidase, | | | | |
| | | | | | | | | | | | | | (ec 1.9.3.1). | the terminal oxidase in | | | | |
| | | | | | | | | | | | | | | mitochondrial electron | | | | |
| | | | | | | | | | | | | | | transport | | | | |
| Msa.1716.0 | 1.00 | 1.52 | −2.53 | 1.21 | −1.25 | 0.14 | −3.16 | 0.63 | −4.17 | 0.61 | cytochrome c | AA028501 | cytochrome c | this protein is one of | 7 68.8 cM | Metabolic | | |
| | | | | | | | | | | | oxidase, subunit | | oxidase polypeptide | the nuclear-coded | | | | |
| | | | | | | | | | | | VIIIb; Cox8b | | viii-heart | polypeptide chains of | | | | |
| | | | | | | | | | | | | | viii-heart | cytochrome c oxidase, | | | | |
| | | | | | | | | | | | | | precursor | the terminal oxidase in | | | | |
| | | | | | | | | | | | | | (ec 1.9.3.1). | mitochondrial electron | | | | |
| | | | | | | | | | | | | | | transport | | | | |
| Msa.26364.0 | 1.00 | −1.67 | −2.77 | 1.15 | −1.53 | 0.32 | −4.00 | 0.27 | −4.18 | 0.77 | mmDNAJA4 | AA062328 | | murine cdna encoding | | Regulatory | | |
| | | | | | | | | | | | | | | a novel type i | | | | |
| | | | | | | | | | | | | | | hsp4/dnaj homolog, | | | | |
| | | | | | | | | | | | | | | mmdja4(1) biochim. | | | | |
| | | | | | | | | | | | | | | biophys. acta 1493 (1- | | | | |
| Msa.27761.0 | 1.00 | 1.08 | −6.28 | 3.32 | −2.67 | 0.08 | −2.84 | 0.35 | −4.39 | 0.45 | ethanol induced 6; | AA068578 | | | | | | |
| | | | | | | | | | | | Etohi6 | | | | | | | |
| X61600 | 1.00 | 1.16 | −2.57 | 1.60 | −1.20 | 0.04 | −11.98 | 5.36 | −4.41 | 1.00 | enolase 3, beta | X61600 | beta enolase (ec | | 11 42.0 cM | Metabolic | | |
| | | | | | | | | | | | muscle; Eno3 | | 4.2.1.11) (2- | | | | | |
| | | | | | | | | | | | | | phospho-d- | | | | | |
| | | | | | | | | | | | | | glycerate | | | | | |
| | | | | | | | | | | | | | hydro-lyase) | | | | | |
| | | | | | | | | | | | | | (skeletal muscle | | | | | |
| | | | | | | | | | | | | | enolase) | | | | | |
| AA688542 | 1.00 | 1.29 | −4.38 | 2.50 | −1.47 | 0.07 | −2.37 | 0.31 | −4.49 | 0.53 | N-myc | AA688542 | ndrg2 protein | | | | | |
| | | | | | | | | | | | downstream | | (ndr2 protein). | | | | | |
| | | | | | | | | | | | regulated 2; | | | | | | | |
| | | | | | | | | | | | Ndr2 | | | | | | | |
| Msa 2491.0 | 1.00 | −2.38 | −6.72 | 3.49 | −2.04 | 0.41 | −3.15 | 0.11 | −4.58 | 0.59 | tenascin X; Tnx | X73959 | | extracellular amtrix | 17 18.74 | ECM | | |
| | | | | | | | | | | | | | | glycoproteins probably | cM | (Matrix | | |
| | | | | | | | | | | | | | | of importance in | | Prot) | | |
| | | | | | | | | | | | | | | regulating | | | | |
| | | | | | | | | | | | | | | developmental | | | | |
| | | | | | | | | | | | | | | processes | | | | |
| AA033394 | 1.00 | 1.52 | −3.15 | 1.22 | −1.53 | 0.19 | −5.64 | 1.78 | −4.61 | 0.82 | muscle glycogen | AA033394 | | | 19 2.0 cM | Metabolic | | |
| | | | | | | | | | | | phosohorylase; Pygm | | | | | | | |
| X67141 | 1.00 | 1.04 | −3.72 | 1.84 | −0.79 | 0.74 | −7.20 | 3.32 | −4.63 | 1.28 | parvalbumin; Pva | X67141 | parvalbumin | in muscle, the calcium- | 15 45.7 cM | Extracellular | | |
| | | | | | | | | | | | | | alpha. | binding protein | | Protein | | |
| | | | | | | | | | | | | | | parvalbumin is | | | | |
| | | | | | | | | | | | | | | thought to be involved | | | | |
| | | | | | | | | | | | | | | in muscle relaxation. | | | | |
| Msa.6099.0 | 1.00 | 1.55 | −3.68 | 1.82 | −0.34 | 1.40 | −6.77 | 1.74 | −4.65 | 1.17 | histidine rich | W13030 | | a striated muscle | 7 20.4 cM | Regulatory | | |
| | | | | | | | | | | | calcium binding | | | sarcoplasmic reticum | | | | |
| | | | | | | | | | | | protein; Hrc | | | | | | | |
| | | | | | | | | | | | | | | (sr)membrane | | | | |
| | | | | | | | | | | | | | | protein. rapid release | | | | |
| | | | | | | | | | | | | | | and uptake of | | | | |
| | | | | | | | | | | | | | | intracellular calcium | | | | |
| | | | | | | | | | | | | | | is the function of the | | | | |
| | | | | | | | | | | | | | | sr. luminal sr proteins | | | | |
| | | | | | | | | | | | | | | are presumed to | | | | |
| | | | | | | | | | | | | | | function in calcium | | | | |
| | | | | | | | | | | | | | | storage and in | | | | |
| | | | | | | | | | | | | | | coordinating calcium | | | | |
| AA611262 | 1.00 | 1.31 | −2.81 | 0.93 | −1.84 | 0.29 | −2.72 | 0.73 | −4.75 | 0.18 | N-myc | AA611262 | ndrg2 protein | a relative of ndr1 | | | | |
| | | | | | | | | | | | downstream | | (ndr2 protein). | (human ndrg1). | | | | |
| | | | | | | | | | | | regulated 2; | | | | | | | |
| | | | | | | | | | | | Ndr2 | | | | | | | |
| Y09257 | 1.00 | −1.07 | −3.31 | 1.28 | −2.41 | 0.78 | −8.29 | 2.99 | −4.78 | 0.84 | nephroblastoma | Y09257 | nov protein | immediate-early | 15 22.5 cM | Signal | | |
| | | | | | | | | | | | overexpressed | | homolog | protein likely to play a | | Transduction | | |
| | | | | | | | | | | | gene; Nov | | (novh). | role in cell growth | | | | |
| | | | | | | | | | | | | | | regulation (by | | | | |
| | | | | | | | | | | | | | | similarity). | | | | |
| Msa.728.0 | 1.00 | 1.48 | −3.39 | 1.24 | −1.69 | 0.49 | −6.12 | 1.21 | −4.91 | 1.03 | solute carrier | M23383 | glucose | insulin-responsive | 11 40.0 cM | Cell Surface | | |
| | | | | | | | | | | | 2 (facilitated | | transporter | glucose carrier protein | | Protein | | |
| | | | | | | | | | | | glucose | | type 4, insulin- | isoform, glut4. | | | | |
| | | | | | | | | | | | transporter), | | responsive (gt2). | specific to achpose | | | | |
| | | | | | | | | | | | member 4; Slc2a4 | | | tissue and to skeletal | | | | |
| | | | | | | | | | | | | | | and cardiac muscle | | | | |
| ET61471 | 1.00 | 1.77 | −5.58 | 1.40 | −5.83 | 2.05 | −3.66 | 0.36 | −4.99 | 0.57 | mast cell protease | ET61471 | mast cell protease | | 17 10.4 cM | | | |
| | | | | | | | | | | | 7; Mcpt7 | | 7 precursor | | | | | |
| | | | | | | | | | | | | | (cc 3 4.21.) | (mmcp-7) | | | | |
| | | | | | | | | | | | | | | (tryptase). | | | | |
| Msa.4287.0 | 1.00 | 1 32 | −3.52 | 1.57 | −1.82 | 0.09 | −6.75 | 1.84 | −5.01 | 0.68 | apolipoprotein B | W29506 | | apobec-2 mrna and | 17 24.0 cM | Intracellular | | |
| | | | | | | | | | | | editing complex 2; | | | protein are expressed | | Protein | | |
| | | | | | | | | | | | Apobec2 | | | exclusively in heart | | | | |
| | | | | | | | | | | | | | | and skeletal muscle. | | | | |
| | | | | | | | | | | | | | | abobec-2 does not | | | | |
| | | | | | | | | | | | | | | display detectable | | | | |
| | | | | | | | | | | | | | | apob mrna editing | | | | |
| | | | | | | | | | | | | | | activity. has low, but | | | | |
| | | | | | | | | | | | | | | definite, intrinsic | | | | |
| | | | | | | | | | | | | | | cytidine deaminase | | | | |
| M76601 | 1.00 | 1.25 | −6.73 | 3.31 | −2.47 | 0.03 | −4.62 | 0.66 | −5.31 | 0.60 | myosin heavy | M76601 | myosin heavy | adult cardiac specific | 14 20.0 cM | Structural | | |
| | | | | | | | | | | | chain cardiac | | chain cardiac | isoform of myosin | | Protein | | |
| | | | | | | | | | | | muscle adult; | | muscle alpha | heavy chain. (see | | | | |
| | | | | | | | | | | | Myhca | | isoform. | additional information | | | | |
| | | | | | | | | | | | | | | for regulation). | | | | |
| X99251 | 1.00 | 1.56 | −1.54 | 1.06 | −1.59 | 0.49 | −4.38 | 1.31 | −5.36 | 1.03 | repetin; Rptn | X99251 | repetin. | novel potential | 3 | | | |
| | | | | | | | | | | | | | | precursor protein of | | | | |
| | | | | | | | | | | | | | | the cornified cell | | | | |
| | | | | | | | | | | | | | | envelop. | | | | |
| Msa.8838.0 | 1.00 | −1.35 | −2.43 | 0.83 | −2.79 | 0.11 | −2.90 | 0.50 | −5.40 | 1.19 | myosin light | W34697 | | | 9 61.0 cM | Structural | | |
| | | | | | | | | | | | chain alkali, | | | | | Protein | | |
| | | | | | | | | | | | cardiac | | | | | | | |
| | | | | | | | | | | | ventricles; Mylc | | | | | | | |
| Msa.13213.0 | 1.00 | 1.37 | −5.96 | 3.66 | −1.84 | 0.19 | −5.66 | 0.99 | −5.68 | 0.86 | actinin alpha 2; | W53582 | | | 13 7.0 cM | Structural | | |
| | | | | | | | | | | | Actn2 | | | | | Protein | | |
| Msa.2776.0 | 1.00 | 1.32 | −3.40 | 1.87 | −1.41 | 0.24 | −2.27 | 0.48 | −6.03 | 1.83 | junction | M90365 | junction | one of the proteins of | 11 60.0 cM | Structural | | |
| | | | | | | | | | | | plakoglobin; Jup | | plakoglobin | desmosomal | | Protein | | |
| | | | | | | | | | | | | | (desmoplakin iii) | membrane anchorage | | | | |
| | | | | | | | | | | | | | (fragment). | site plaques of the | | | | |
| | | | | | | | | | | | | | | epithelium, and is also | | | | |
| | | | | | | | | | | | | | | a component of | | | | |
| | | | | | | | | | | | | | | plaques of the | | | | |
| | | | | | | | | | | | | | | adhering junction | | | | |
| AA562768 | 1.00 | 2.21 | −7.37 | 1.45 | −3.74 | 2.31 | −7.62 | 2.96 | −6.40 | 1.95 | glioblastoma | AA562768 | | | | Other | | |
| | | | | | | | | | | | amplified sequence; | | | | | | | |
| | | | | | | | | | | | Gbas | | | | | | | |
| Msa.2946.0 | 1.00 | 1.13 | −4.57 | 1.95 | −2.15 | 0.90 | −2.87 | 0.34 | −6.70 | 0.89 | cysteine-rich | W08774 | ccaat/enhancer | cardiac lim protein | | Transcription | | |
| | | | | | | | | | | | protein 3; Carp3 | | binding protein | | | Factor | | |
| | | | | | | | | | | | | | delta (c/ebp delta) | | | | | |
| | | | | | | | | | | | | | (c/ebp-related | | | | | |
| | | | | | | | | | | | | | protein 3).,lim | | | | | |
| | | | | | | | | | | | | | domain protein, | | | | | |
| | | | | | | | | | | | | | cardiac (muscle lim | | | | | |
| | | | | | | | | | | | | | protein) (cysteine- | | | | | |
| | | | | | | | | | | | | | rich protein 3) | | | | | |
| Msa.727.0 | 1.00 | 1.74 | −5.46 | 1.82 | −3.53 | 0.55 | −5.48 | 1.18 | −6.92 | 1.19 | glutathione-S- | M73483 | glutathione s- | conjugation of | 9 48.0 cM | Metabolic | | |
| | | | | | | | | | | | transferase, | | transferase yc (ec | reduced glutathione to | | | | |
| | | | | | | | | | | | alpha 3; Gsta3 | | 2.5.1.18)(gst class- | a wide number of | | | | |
| | | | | | | | | | | | | | alpha). | exogenous and | | | | |
| | | | | | | | | | | | | | | endogenous | | | | |
| | | | | | | | | | | | | | | hydrophobic | | | | |
| | | | | | | | | | | | | | | electrophiles. this gst | | | | |
| | | | | | | | | | | | | | | has a high catalytic | | | | |
| | | | | | | | | | | | | | | activity for aflatoxin | | | | |
| Maa.27462.0 | 1.00 | −1.46 | −4.50 | 1.10 | −4.40 | 1.47 | −4.02 | 1.38 | −7.09 | 1.40 | growth hormone | AA066700 | high molecular | binding of gh to ghr | 15 4 6 cM | Receptor | | |
| | | | | | | | | | | | receptor; Ghr | | weight growth | activates insulin-like | | | | |
| | | | | | | | | | | | | | hormone | growth factor 1 (igf1), | | | | |
| | | | | | | | | | | | | | receptor/binding | which in turn binds to | | | | |
| | | | | | | | | | | | | | protein | its own receptor to | | | | |
| | | | | | | | | | | | | | precuraor.,low | activate signal- | | | | |
| | | | | | | | | | | | | | molecular weight | transduction pathways | | | | |
| | | | | | | | | | | | | | growth hormone | leading to growth | | | | |
| | | | | | | | | | | | | | receptor/binding | | | | | |
| | | | | | | | | | | | | | protein precursor | | | | | |
| Msa.540.0 | 1.00 | 1.21 | −5.09 | 2.84 | −1.82 | 0.51 | −5.41 | 1.45 | −7.23 | 1.37 | gap junction | M91443 | gap junction beta-4 | one gap junction | 4 57.5 cM | | | |
| | | | | | | | | | | | membrane | | protein (connexin | consists of a cluster of | | | | |
| | | | | | | | | | | | channel | | 31.1) (cx31.1). | closely packed pairs of | | | | |
| | | | | | | | | | | | protein beta 4; | | | transmembrane | | | | |
| | | | | | | | | | | | Gjb4 | | | channels, the | | | | |
| | | | | | | | | | | | | | | connnexons, through | | | | |
| | | | | | | | | | | | | | | which materials of low | | | | |
| | | | | | | | | | | | | | | mw diffuse from one | | | | |
| | | | | | | | | | | | | | | cell to a neighboring | | | | |
| | | | | | | | | | | | | | | cell | | | | |
| Msa.4623.0 | 1.00 | 1.37 | −6.95 | 3.61 | −1.82 | 0.25 | −8.69 | 2.10 | −7.51 | 0.45 | actinin alpha 2; | W34429 | | | 13 7.0 cM | Structural | | |
| | | | | | | | | | | | Actn2 | | | | | Protein | | |
| X91825 | 1.00 | 1.35 | −3.03 | 1.34 | −2.07 | 0.56 | −5.10 | 0.64 | −7.86 | 1.18 | small proline-rich | X91825 | cornifin b (small | cross-linked envelop | 3 45.2 cM | | | |
| | | | | | | | | | | | protein IB; | | proline-rich | protein of | | | | |
| | | | | | | | | | | | Sprr1b | | protein 1b) | keratinocytes. it is a | | | | |
| | | | | | | | | | | | | | (spr1b)(spr1 b). | keratinocyte protein | | | | |
| | | | | | | | | | | | | | | that first appears in the | | | | |
| | | | | | | | | | | | | | | cell cytosol, but | | | | |
| | | | | | | | | | | | | | | ultimately becomes | | | | |
| | | | | | | | | | | | | | | cross-linked to | | | | |
| | | | | | | | | | | | | | | membrane proteins by | | | | |
| | | | | | | | | | | | | | | transglutaminase. all | | | | |
| | | | | | | | | | | | | | | that results in the | | | | |
| | | | | | | | | | | | | | | formation of an | | | | |
| | | | | | | | | | | | | | | insoluble envelop | | | | |
| | | | | | | | | | | | | | | beneath the plasma | | | | |
| Msa.17804.0 | 1.00 | −5.76 | −4.91 | 2.21 | −1.68 | 0.14 | −4.53 | 1.89 | −8.12 | 1.86 | synuclein, | AA108571 | | | 14 12.5 cM | | | |
| | | | | | | | | | | | Sncg | | | | | | | |
| Msa.9519.0 | 1.00 | 1.22 | −10.56 | 7.86 | −1.87 | 0.70 | −10.02 | 4.21 | −8.45 | 0.47 | actinin alpha 2; | W40754 | | alpha-actinin 2 mrna, | 13 7.0 cM | Structural | | |
| | | | | | | | | | | | Actn2 | | | | | Protein | | |
| Msa.22711.0 | 1.00 | −1.16 | −1.54 | 1.75 | 1.07 | 0.03 | −9.74 | 1.39 | −8.51 | 2.35 | creatine kinase; | AA038095 | | reversibly catalyzes | | Metabolic | | |
| | | | | | | | | | | | mitochondrial 2 | | | the transfer of | | | | |
| | | | | | | | | | | | (sarcomeric) | | | phosphate between atp | | | | |
| | | | | | | | | | | | | | | and various | | | | |
| | | | | | | | | | | | | | | phosphogens (e.g. | | | | |
| | | | | | | | | | | | | | | creatine phosphate). | | | | |
| | | | | | | | | | | | | | | creatine kinase | | | | |
| | | | | | | | | | | | | | | isoenzymes play a | | | | |
| | | | | | | | | | | | | | | central role in energy | | | | |
| | | | | | | | | | | | | | | transduction in tissues | | | | |
| | | | | | | | | | | | | | | with large, fluctuating | | | | |
| | | | | | | | | | | | | | | energy demands, such | | | | |
| Msa 20143.0 | 1.00 | 1.25 | −2.76 | 2.09 | −0.75 | 1.88 | −4.08 | 0.93 | −9.86 | 1.89 | hydroxysteroid | AA016485 | | | | | | |
| | | | | | | | | | | | sulfotransferase, | | | | | | | |
| | | | | | | | | | | | SULT2B | | | | | | | |
| Msa.4317.0 | 1.00 | 2.43 | −6.26 | 3.80 | −1.72 | 0.38 | −2.62 | 0.21 | −11.22 | 4.05 | calcium channel, | AA061886 | | | | Cell Surface | | |
| | | | | | | | | | | | voltage-dependent, | | | | | Protein | | |
| | | | | | | | | | | | gamma subunit 1; | | | | | | | |
| | | | | | | | | | | | Cacng1 | | | | | | | |
| Mss.24682.0 | 1.00 | 1.46 | −4.33 | 1.40 | −2.98 | 0.65 | −9.23 | 0.73 | −11.22 | 1.05 | ART3 (ADP- | W82798 | | genecards: testis | | Intracellular | | |
| | | | | | | | | | | | ribosyltransferase | | | specific. | | Protein | | |
| | | | | | | | | | | | 3) | | | | | | | |
| M88694 | 1.00 | 15.59 | −3.05 | 2.20 | −0.50 | 1.50 | −6.42 | 4.80 | −11.31 | 4.10 | thioether S- | M88694 | thioether s- | catalyzes transfer of | | Other | | |
| | | | | | | | | | | | methyltransferase, | | methyltransferase | the methyl group from | | | | |
| | | | | | | | | | | | Temt | | (ec 2.1.1.96) | s-adenosylmethionine | | | | |
| | | | | | | | | | | | | | (tcmt) | to x in compounds of | | | | |
| | | | | | | | | | | | | | | the structure r-x-r′, | | | | |
| | | | | | | | | | | | | | | where x may be sulfur, | | | | |
| | | | | | | | | | | | | | | selenium, or tellurium, | | | | |
| | | | | | | | | | | | | | | and r and r′ may be | | | | |
| | | | | | | | | | | | | | | various organic | | | | |
| X79199 | 1.00 | −4.39 | −3.08 | 2.24 | −1.82 | 0.28 | −7.10 | 3.28 | −11.54 | 3.71 | tetranectin | X79199 | tetranectin | aka plasminogen | 9 71.0 cM | ECM | | |
| | | | | | | | | | | | (plasminogen- | | precursor (m) | binding protein-a | | (Matrix | | |
| | | | | | | | | | | | binding | | (plasminogen | plasminogen-binding | | Prot) | | |
| | | | | | | | | | | | protein); Tna | | kringle 4 binding | protein with a c-type | | | | |
| | | | | | | | | | | | | | protein). | lectin domain, is | | | | |
| | | | | | | | | | | | | | | found in both serum | | | | |
| | | | | | | | | | | | | | | and the extracellular | | | | |
| | | | | | | | | | | | | | | matrix. it is a | | | | |
| | | | | | | | | | | | | | | matricellular protein | | | | |
| | | | | | | | | | | | | | | and plays a role in | | | | |
| X83932 | 1.00 | 2.28 | −3.02 | 1.55 | −0.27 | 1.54 | −1.90 | 0.20 | −15.56 | 13.50 | ryanodine | X83932 | | provides a release | 7 10.0 cM | Receptor | | |
| | | | | | | | | | | | receptor 1 | | | mechanism for | | | | |
| | | | | | | | | | | | skeletal muscle | | | internal cellular ca2+. | | | | |
| | | | | | | | | | | | Ryr1 | | | mutation associated | | | | |
| | | | | | | | | | | | | | | with human malignant | | | | |
| | | | | | | | | | | | | | | hyperthermia | | | | |
| | | | | | | | | | | | | | | (mh)(omim 1456). | | | | |
| | | | | | | | | | | | | | | ryr1 is predominant in | | | | |
| | | | | | | | | | | | | | | skeletal muscle, but is | | | | |
| | | | | | | | | | | | | | | also detectable in | | | | |
| | | | | | | | | | | | | | | heart and in brain | | | | |
| M91602 | 1.00 | −1.04 | −6.76 | 4.34 | −1.48 | 0.21 | −18.83 | 7.24 | −16.81 | 3.83 | myosin light | M91602 | myosin regulatory | a regulatory light | | Structural | | |
| | | | | | | | | | | | chain, | | light chain 2, | chain predominantly | | Protein | | |
| | | | | | | | | | | | phosphorylatable, | | ventricular/cardiac | expressed in | | | | |
| | | | | | | | | | | | cardiac ventricles; | | muscle isoform | ventricular cardiac | | | | |
| | | | | | | | | | | | Mylpc | | (mlc-2) | muscle | | | | |
| Msa.1007.0 | 1.00 | 1.20 | −10.87 | 7.61 | −1.90 | 0.38 | −26.29 | 5.63 | −17.28 | 4.06 | myosin light | M91602 | myosin regulatory | a regulatory light | | Structural | | |
| | | | | | | | | | | | chain, | | light chain 2, | chain predominantly | | Protein | | |
| | | | | | | | | | | | phosphorylatable | | ventricular/cardiac | expressed in | | | | |
| | | | | | | | | | | | cardiac ventricles | | muscle isoform | ventricular cardiac | | | | |
| | | | | | | | | | | | Mylpc | | (mlc-2) | muscle | | | | |
| M29793 | 1.00 | 1.28 | −6.52 | 3.56 | −2.79 | 0.98 | −19.87 | 14.88 | −18.50 | 9.76 | troponin C, | M29793 | troponin c, slow | troponin is the central | 14 10.0 cM | Structural | | |
| | | | | | | | | | | | cardiac/slow | | skeletal and | regulatory protein of | | Protein | | |
| | | | | | | | | | | | skeletal; Tncc | | cardiac | striated muscle | | | | |
| | | | | | | | | | | | | | muscles (tn-c) | contraction, tn consists | | | | |
| | | | | | | | | | | | | | | of three components: | | | | |
| | | | | | | | | | | | | | | tn-i which is the | | | | |
| | | | | | | | | | | | | | | inhibitor of | | | | |
| | | | | | | | | | | | | | | actomyosin atpase, tn- | | | | |
| | | | | | | | | | | | | | | t which contain the | | | | |
| | | | | | | | | | | | | | | binding site for | | | | |
| | | | | | | | | | | | | | | tropomyosin and tn-c. | | | | |
| | | | | | | | | | | | | | | the binding of calcium | | | | |

1. A computer-readable medium comprising a plurality of digitally encoded values representing the levels of expression of a plurality of genes characteristic of R.A. including a plurality of genes selected from the group consisting of SOCS3 (CISH3); RAGE (AGER); LST-1 (LY117); serum amyloid (SAA) 1-3; HMG-1; S100 A8, A9, and A12; Secretory Leukocyte Protease Inhibitor (SLPI); glucocorticoid leucine zipper (GILZ); PTPN-18; GADD-45A and B; Legumain (PRSC1); follistatin-like 1 (FST1); lipocalin 2 (Lcn2); glucose phosphate isomerase (GPI); Serine Protease Inhibitor (SpiL); and TSG-6, in a cell characteristic of R.A.

2. The computer-readable medium of claim 1, comprising values representing levels of expression of at least 5 genes selected from the group consisting of SOCS3 (CISH3); RAGE (AGER); LST-1 (LY117); SAA 1-3; HMG-1; S100 A8, A9, and A12; SLPI; GILZ; PTPN-18; GADD-45A and B; Legumain (PRSCl); FST1; Lcn2; GPI; SpiL; and TSG-6.

3. A computer-readable medium comprising a plurality of digitally encoded values representing the levels of expression of at least 10 genes characteristic of R.A. in a cell characteristic of R.A.

4. The computer-readable medium of claim 3, comprising values representing levels of expression of at least 50% of the genes set forth in Tables 1-5.

5. The computer-readable medium of claim 1, further comprising at least one value representing a level of expression of at least one gene characteristic of R.A. in a normal counterpart cell.

6. The computer-readable medium of claim 1, wherein the values represent ratios of, or differences between, a level of expression of a gene characteristic of R.A. in a cell characteristic of R.A. and a level of expression of the gene in a normal counterpart cell.

7. The computer-readable medium of claim 1, wherein less than about 50% of the values on the computer-readable medium represent expression levels of genes which are not characteristic of R.A.

8. A computer system, comprising:
a database comprising values representing expression levels of a plurality of genes characteristic of R.A. including a plurality of genes selected from the group consisting of SOCS3 (CISH3); RAGE (AGER); LST-1 (LY117); SAA 1-3; HMG-1; S100 A8, A9, and A12; SLPI; GILZ; PTPN-18; GADD-45A and B; Legumain (PRSC1); FST1; Lcn2; GPI;
SpiL; and TSG-6, in a cell characteristic of R.A.; and,
a processor having instructions to,
receive at least one query value representing at least one level of expression of at least one gene represented in the database, and,
compare the at least one query value and the at least one database value.

9. A computer program for analyzing levels of expression of a plurality of genes characteristic of R.A. in a cell, the computer program being disposed on a computer readable medium and including instructions for causing a processor to:
receive query values representing levels of expression of a plurality of genes characteristic of R.A. in a cell, and,
compare the query values with levels of expression of the plurality of genes in a cell characteristic of R.A.

10. A composition comprising a plurality of detection agents of genes which are characteristic of R.A. including a plurality of genes selected from the group consisting of SOCS3 (CISH3); RAGE (AGER); LST-1 (LY117); SAA 1-3; HMG-1; S100 A8, A9, and A12; SLPI; GILZ; PTPN-18; GADD-45A and B; Legumain (PRSC1); FST1; Lcn2; GPI; SpiL; and TSG-6, which detection agents are capable of detecting the expression of the genes or the polypeptides encoded by the genes, and wherein less than about 50% of the detection agents are genes which are not characteristic of R.A.

11. The composition of claim 10, wherein the detection agents are isolated nucleic acids which hybridize specifically to nucleic acids corresponding to the genes.

12. The composition of claim 12, comprising isolated nucleic acids which hybridize specifically to at least five genes selected from the group consisting of SOCS3 (CISH3); RAGE (AGER); LST-1 (LY117); SAA 1-3; HMG-1; S1OO A8, A9, and A12; SLPI; GILZ; PTPN-18; GADD-45A and B; Legumain (PRSC1); FST1; Lcn2; GPI; SpiL; and TSG-6.

13. The composition of claim 10, comprising isolated nucleic acids which hybridize specifically to at least 10 different genes characteristic of R.A.

14. The composition of claim 13, comprising isolated nucleic acids which hybridize specifically to at least 100 different genes characteristic of R.A.

15. A solid surface to which are linked a plurality of detection agents of genes which are characteristic of R.A. including a plurality of genes selected from the group consisting of SOCS3 (CISH3); RAGE (AGER); LST-1 (LY117); SAA 1-3; HMG-1; S100 A8, A9, and A12; SLPI; GILZ; PTPN-18; GADD-45A and B; Legumain (PRSC1); FST1; Lcn2; GPI; SpiL; and TSG-6, which detection agents are capable of detecting the expression of the genes or the polypeptides encoded by the genes, and wherein less than about 50% of the detection agents on the solid surface are not detecting genes characteristic of R.A.

16. The solid surface of claim 15, wherein the detection agents are isolated nucleic acids which hybridize specifically to the genes.

17. The solid surface of claim 16, wherein the detection agents are covalently linked to the solid surface.

18. A composition comprising antagonists of a plurality of genes characteristic of R.A. including a plurality of genes selected from the group consisting of SOCS3 (CISH3); RAGE (AGER); LST-1 (LY117); SAA 1-3; HMG-1; S100 A8, A9, and A12; SLPI; GILZ; PTPN-18; GADD-45A and B; Legumain (PRSC1); FST1; Lcn2; GPI; SpiL; and TSG-6.

19. The composition of claim 18, wherein the antagonists are antisense nucleic acids, siRNAs, ribozymes or dominant negative mutants.

20. A method for determining the difference between levels of expression of a plurality of genes characteristic of R.A. in a cell and reference levels of expression of the genes, comprising
providing RNA from a cell;
determining levels of RNA of a plurality of genes characteristic of R.A. including a plurality of genes selected from the group consisting of SOCS3 (CISH3); RAGE (AGER); LST-1 (LY117); SAA 1-3; HMG-1; S100 A8, A9, and A12; SLPI; GILZ; PTPN-18; GADD-45A and B; Legumain (PRSC1); FST1; Lcn2; GPI; SpiL; and TSG-6 to obtain the levels of expression of the plurality of genes in the cell; and
comparing the levels of expression of the plurality of genes in the cell to a set of reference levels of expression of the genes,
to thereby determine the difference between levels of expression of the plurality of genes characteristic of R.A. in the cell and reference levels of expression of the genes.

21. The method of claim 20, wherein the set of reference levels of expression includes the levels of expression of the genes in a subject having R.A.

22. The method of claim 21, wherein the set of reference levels of expression further includes the levels of expression of the genes in a subject who does not have R.A.

23. The method of claim 20, comprising incubating a nucleic acid sample derived from the RNA of the cell of the subject with nucleic acids corresponding to the genes, under conditions wherein two complementary nucleic acids hybridize to each other.

24. The method of claim 23, wherein the nucleic acids corresponding to the genes are attached to a solid surface.

25. The method of claim 20, comprising entering the levels of expression of the plurality of genes into a computer which comprises a memory with values representing the set of reference levels of expression.

26. The method of claim 25, wherein comparing the level comprises providing computer instructions to perform.

27. A method for determining whether a subject has or is likely to develop R.A., comprising obtaining a cell from the subject and comparing gene expression levels in the cell to those of a set of reference levels of expression, according to the method of claim 20, wherein similar levels of expression of the plurality of genes indicates that the subject has or is likely to develop R.A.

28. The method of claim 27, wherein the cell is a peripheral blood mononuclear cell (PBMC) and the set of reference levels of expression includes the levels of expression of the genes in a PBMC of a subject having R.A.

29. The method of claim 27, wherein the cell is a PBMC and the set of reference levels of expression includes the average of levels of expression of the genes in a PBMC of a plurality of subjects having R.A.

30. The method of claim 27, further comprising iteratively providing RNA from the subject and determining the level of RNA, such as to determine an evolution of the levels of expression of the genes in the subject.

31. A method for determining whether a therapy for R.A. is effective in a subject having R.A. who is receiving the therapy, comprising obtaining a cell from the subject and comparing levels of expression in the cell of the subject to those in subjects having R.A. and in subjects who do not have R.A., according to the method of claim 20, wherein levels of expression in the cell of the subject that are more similar to those of the subject having R.A. than the subject who does not have R.A. indicates that the therapy is not effective, whereas levels of expression in the cell of the subject that are more similar to those of the subject not having R.A. than the subject having R.A. indicates that the therapy is effective.

32. The method of claim 27, wherein the set of reference levels of expression is in the form of a database.

33. The method of claim 32, wherein the database is included in a computer-readable medium.

34. The method of claim 33, wherein the database is in communications with a microprocessor and microprocessor instructions for providing a user interface to receive expression level data of a subject and to compare the expression level data with the database.

35. The method of claim 27, comprising
obtaining a patient sample from a caregiver;
identifying expression levels of a plurality of genes characteristic of R.A. from the patient sample;
determining whether the levels of expression of the genes in the patient sample are more similar to those of a subject having R.A. or to those of a subject who does not have R.A.; and
transmitting the results to the caregiver.

36. The method of claim 35, wherein the results are transmitted across a network.

37. A method for identifying a compound for treating R.A., comprising
providing levels of expression of a plurality of genes characteristic of R.A. in a cell characteristic of R.A. incubated with a test compound;
providing levels of expression of a normal counterpart cell; and
comparing the two levels of expression, wherein similar levels of expression in the two cells indicates that the compound is likely to be effective for treating R.A.

38. A diagnostic or drug discovery kit, comprising a computer-readable medium of claim 1 and instructions for use.

39. A diagnostic or drug discovery kit, comprising a composition of claim 10 and instructions for use.

40. A diagnostic or drug discovery kit, comprising a solid surface of claim 15 and instructions for use.
